(19) United States
(12) Patent Application Publication
(10) Pub. No.: US 2024/0251578 A1
(43) Pub. Date: Jul. 25, 2024

(54) COMPOUND FOR ORGANIC OPTOELECTRONIC DEVICE, COMPOSITION FOR ORGANIC OPTOELECTRONIC DEVICE, ORGANIC OPTOELECTRONIC DEVICE, AND DISPLAY DEVICE

(71) Applicant: SAMSUNG SDI CO., LTD., Yongin-si, Gyeonggi-do (KR)

(72) Inventors: Byungku KIM, Suwon-si, Gyeonggi-do (KR); Yunsoo KIM, Suwon-si, Gyeonggi-do (KR); Sunwoong SHIN, Suwon-si, Gyeonggi-do (KR); Byoungkwan LEE, Suwon-si, Gyeonggi-do (KR); Kipo JANG, Suwon-si, Gyeonggi-do (KR); Sung-Hyun JUNG, Suwon-si, Gyeonggi-do (KR); Jongwoo CHOI, Suwon-si, Gyeonggi-do (KR); Hyunjung KIM, Suwon-si, Gyeonggi-do (KR); Mijin LEE, Suwon-si, Gyeonggi-do (KR)

(21) Appl. No.: 18/288,439

(22) PCT Filed: Dec. 5, 2022
(86) PCT No.: PCT/KR2022/019557

§ 371 Date: Oct. 26, 2023

(30) Foreign Application Priority Data
Dec. 16, 2021 (KR) 10-2021-0180991

(51) Int. Cl. H10K 50/11; H10K 85/30; H10K 85/60

(52) U.S. Cl. CPC H10K 50/11; H10K 85/346; H10K 85/633; H10K 85/654; H10K 85/6572; H10K 85/6574; H10K 85/6576

(57) ABSTRACT

Provided are a compound for an organic optoelectronic device represented by Chemical Formula 1, a composition for an organic optoelectronic device including the same, an organic optoelectronic device, and a display device.

The contents of Chemical Formula I are as defined in the specification.

### TECHNICAL FIELD

A compound for an organic optoelectronic device, a composition for an organic optoelectronic device, an organic optoelectronic device, and a display device are disclosed.

### BACKGROUND ART

An organic optoelectronic device (organic optoelectronic diode) is a device capable of converting electrical energy and optical energy to each other.

Organic optoelectronic devices may be largely divided into two types according to a principle of operation. One is a photoelectric device that generates electrical energy by separating excitons formed by light energy into electrons and holes, and transferring the electrons and holes to different electrodes, respectively and the other is light emitting device that generates light energy from electrical energy by supplying voltage or current to the electrodes.

Examples of the organic optoelectronic device include an organic photoelectric device, an organic light emitting diode, an organic solar cell, and an organic photoconductor drum.

Among them, organic light emitting diodes (OLEDs) are attracting much attention in recent years due to increasing demands for flat panel display devices. The organic light emitting diode is a device that converts electrical energy into light, and the performance of the organic light emitting diode is greatly influenced by an organic material between electrodes.

### DISCLOSURE

### Technical Problem

An embodiment provides a compound for an organic optoelectronic device capable of implementing a high efficiency and long life-span organic optoelectronic device.

Another embodiment provides a composition for an organic optoelectronic device including the compound for an organic optoelectronic device.

Another embodiment provides an organic optoelectronic device including the compound for an organic optoelectronic device or the composition for an organic optoelectronic device.

Another embodiment provides a display device including the organic optoelectronic device.

### Technical Solution

According to an embodiment, a compound for an organic optoelectronic device represented by Chemical Formula 1 is provided.

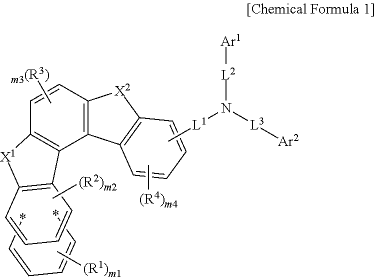

In Chemical Formula 1,
X¹ and X² are each independently O or S,
L¹ to L³ are each independently a single bond or a substituted or unsubstituted C6 to C20 arylene group,
Ar¹ and Ar² are each independently a substituted or unsubstituted C6 to C20 aryl group or a substituted or unsubstituted C2 to C30 heterocyclic group,
R¹ to R⁴ are each independently hydrogen, deuterium, a halogen, a substituted or unsubstituted C1 to C10 alkyl group or a substituted or unsubstituted C6 to C12 aryl group,
m1 is one of integers of 1 to 4,
m2 and m3 are each independently an integer of 1 or 2, and
m4 is one of integers of 1 to 3.

According to another embodiment, a composition for an organic optoelectronic device includes a first compound and a second compound.

The first compound is as described above, and the second compound may be a compound for an organic optoelectronic device represented by Chemical Formula 2.

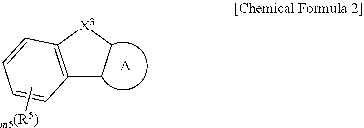

In Chemical Formula 2,
X³ is O, S, N—L^{a}—R^{a}, CR^{b}R^{c}, or SiR^{d}R^{e},
L^{a} is a single bond, or a substituted or unsubstituted C6 to C12 arylene group,
R^{a}, R^{b}, R^{c}, R^{d}, R^{e}, and R⁵ are each independently hydrogen, deuterium, a substituted or unsubstituted C6 to C30 aryl group or a substituted or unsubstituted C2 to C30 heterocyclic group,
m5 is one of integers of 1 to 4, and

A is any one selected from the rings listed in Group II,

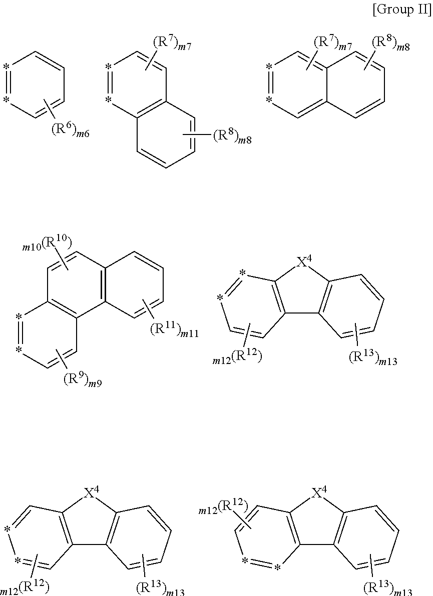

In Group II,
is a linking point,
X⁴ is O or S,
R⁶ to R¹³ are each independently hydrogen, deuterium, a substituted or unsubstituted C6 to C20 aryl group, or a substituted or unsubstituted C2 to C30 heterocyclic group,
m6, m8, m11, and m13 are each independently one of integers of 1 to 4,
m7, m9, m10, and m12 are each independently an integer of 1 or 2, and
at least one of R^{a} and R⁵ to R¹³ is a group represented by Chemical Formula a,

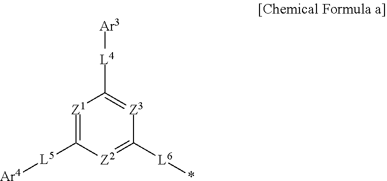

wherein, in Chemical Formula a,
Z¹ to Z³ are each independently N or CR^{f},
at least two of Z¹ to Z³ are N,
R^{f} is hydrogen, deuterium, a substituted or unsubstituted C1 to C30 alkyl group, or a substituted or unsubstituted C6 to C30 aryl group,
L⁴ to L⁶ are each independently a single bond, or a substituted or unsubstituted C6 to C30 arylene group,
Ar³ and Ar⁴ are each independently a substituted or unsubstituted C6 to C30 aryl group, or a substituted or unsubstituted C2 to C30 heteroaryl group, and
is a linking point.

According to another embodiment, an organic optoelectronic device includes an anode and a cathode facing each other, and at least one organic layer between the anode and the cathode, and the organic layer includes the compound for an organic optoelectronic device or the composition for an organic optoelectronic device.

According to another embodiment, a display device including the organic optoelectronic device is provided.

### Advantageous Effects

An organic optoelectronic device having high efficiency and a long life-span may be realized.

### DESCRIPTION OF THE DRAWINGS

FIG. 1 is a cross-sectional view showing an organic light emitting diode according to an embodiment.

### DESCRIPTION OF SYMBOLS

100: organic light emitting diode
105: organic layer
110: cathode
120: anode
130: light emitting layer
140: hole transport region
150: electron transport region

### MODE FOR INVENTION

Hereinafter, embodiments of the present invention are described in detail. However, these embodiments are exemplary, the present invention is not limited thereto and the present invention is defined by the scope of claims.

As used herein, when a definition is not otherwise provided, “substituted” refers to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 trifluoroalkyl group, a cyano group, or a combination thereof.

In one example of the present invention, the “substituted” refers to replacement of at least one hydrogen of a substituent or a compound by deuterium, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, or a cyano group. In specific example of the present invention, the “substituted” refers to replacement of at least one hydrogen of a substituent or a compound by deuterium, a C1 to C20 alkyl group, a C6 to C30 aryl group, or a cyano group. In specific example of the present invention, the “substituted” refers to replacement of at least one hydrogen of a substituent or a compound by deuterium, a C1 to C5 alkyl group, a C6 to C18 aryl group, or a cyano group. In specific example of the present invention, the “substituted” refers to replacement of at least one hydrogen of a substituent or a compound by deuterium, a cyano group, a methyl group, an ethyl group, a propyl group, a butyl group, a phenyl group, a biphenyl group, a terphenyl group, or a naphthyl group.

“Unsubstituted” refers to non-replacement of a hydrogen atom by another substituent and remaining of the hydrogen atom.

In the present specification, “hydrogen (—H)” may include “deuterium substitution (-D)” or “tritium substitution (-T).”

As used herein, when a definition is not otherwise provided, “hetero” refers to one including one to three heteroatoms selected from N, O, S, P, and Si, and remaining carbons in one functional group.

As used herein, “aryl group” refers to a group including at least one hydrocarbon aromatic moiety, and all elements of the hydrocarbon aromatic moiety have p-orbitals which form conjugation, for example a phenyl group, a naphthyl group, and the like, two or more hydrocarbon aromatic moieties may be linked by a sigma bond and may be, for example a biphenyl group, a terphenyl group, a quarterphenyl group, and the like, and two or more hydrocarbon aromatic moieties are fused directly or indirectly to provide a non-aromatic fused ring, for example a fluorenyl group.

The aryl group may include a monocyclic, polycyclic, or fused ring polycyclic (i.e., rings sharing adjacent pairs of carbon atoms) functional group.

As used herein, “a heterocyclic group” is a generic concept of a heteroaryl group, and may include at least one heteroatom selected from N, O, S, P, and Si instead of carbon (C) in a cyclic compound such as aryl group, a cycloalkyl group, a fused ring thereof, or a combination thereof. When the heterocyclic group is a fused ring, the entire ring or each ring of the heterocyclic group may include one or more heteroatoms.

For example, “a heteroaryl group” may refer to aryl group including at least one heteroatom selected from N, O, S, P, and Si. Two or more heteroaryl groups are linked by a sigma bond directly, or when the heteroaryl group includes two or more rings, the two or more rings may be fused. When the heteroaryl group is a fused ring, each ring may include one to three heteroatoms.

More specifically, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted triphenylene group, a substituted or unsubstituted perylenyl group, a substituted or unsubstituted fluorenyl group, a substituted or unsubstituted indenyl group, a substituted or unsubstituted furanyl group, or a combination thereof, but is not limited thereto.

More specifically, the substituted or unsubstituted C2 to C30 heterocyclic group may be a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzthiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted carbazolyl group, a substituted or unsubstituted dibenzofuranyl group, a substituted or unsubstituted dibenzothiophenyl group, a substituted or unsubstituted benzofuranpyrimidinyl group, a substituted or unsubstituted benzothiophenepyrimidinyl group, or a combination thereof, but is not limited thereto.

As used herein, hole characteristics refer to an ability to donate an electron to form a hole when an electric field is applied and that a hole formed in the anode may be easily injected into the light emitting layer and transported in the light emitting layer due to conductive characteristics according to a highest occupied molecular orbital (HOMO) level.

In addition, electron characteristics refer to an ability to accept an electron when an electric field is applied and that electron formed in the cathode may be easily injected into the light emitting layer and transported in the light emitting layer due to conductive characteristics according to a lowest unoccupied molecular orbital (LUMO) level.

Hereinafter, a compound for an organic optoelectronic device according to an embodiment is described.

The compound for an organic optoelectronic device according to an embodiment is represented by Chemical Formula 1.

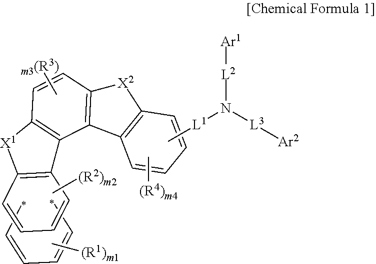

In Chemical Formula 1,
X¹ and X² are each independently O or S,
L¹ to L³ are each independently a single bond or a substituted or unsubstituted C6 to C20 arylene group,
Ar¹ and Ar² are each independently a substituted or unsubstituted C6 to C20 aryl group or a substituted or unsubstituted C2 to C30 heterocyclic group,
R¹ to R⁴ are each independently hydrogen, deuterium, a halogen, a substituted or unsubstituted C1 to C10 alkyl group or a substituted or unsubstituted C6 to C12 aryl group,
m1 is one of integers of 1 to 4,
m2 and m3 are each independently an integer of 1 or 2, and
m4 is one of integers of 1 to 3.

In the compound represented by Chemical Formula 1, dibenzofuran (or dibenzothiophene) is fused with naphthofuran (or naphthothiophene), thereby increasing a planarity of the molecule increases and improving stacking of the lattice, and thus increasing the glass transition temperature and hole mobility. Additionally, as it is substituted with the amine group, hole injection and hole movement become faster.

The high glass transition temperature resulting from these structural features ensures stable device characteristics by maintaining a stable film even against Joule heat generated during device operation, enabling the implementation of devices with excellent life-span, and high hole mobility and fast hole injection characteristics improve the driving voltage of the device.

In Chemical Formula 1, when m1 is 2 or more, each R¹ may be the same or different from each other.

In Chemical Formula 2, when m2 is 2 or more, each R² may be the same or different from each other.

In Chemical Formula 1, when m3 is 2 or more, each R³ may be the same or different from each other.

In Chemical Formula 1, when m4 is 2 or more, each R⁴ may be the same or different from each other.

As an example, Chemical Formula 1 may be represented by any one of Chemical Formula 1A, Chemical Formula 1B, and Chemical Formula 1C.

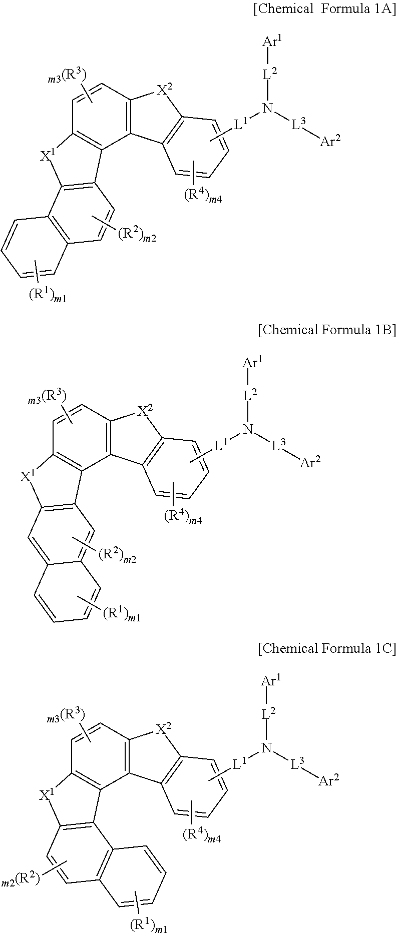

In Chemical Formula 1A, Chemical Formula 1B, and Chemical Formula 1C,
X¹, X², L¹ to L³, Ar¹, Ar², R¹ to R⁴, and m1 to m4 are the same as described above.

As a specific example, Chemical Formula 1A may be represented by one of Chemical Formula 1A-1, Chemical Formula 1A-2, Chemical Formula 1A-3, and Chemical Formula 1A-4, depending on the specific substitution position of the amine group.

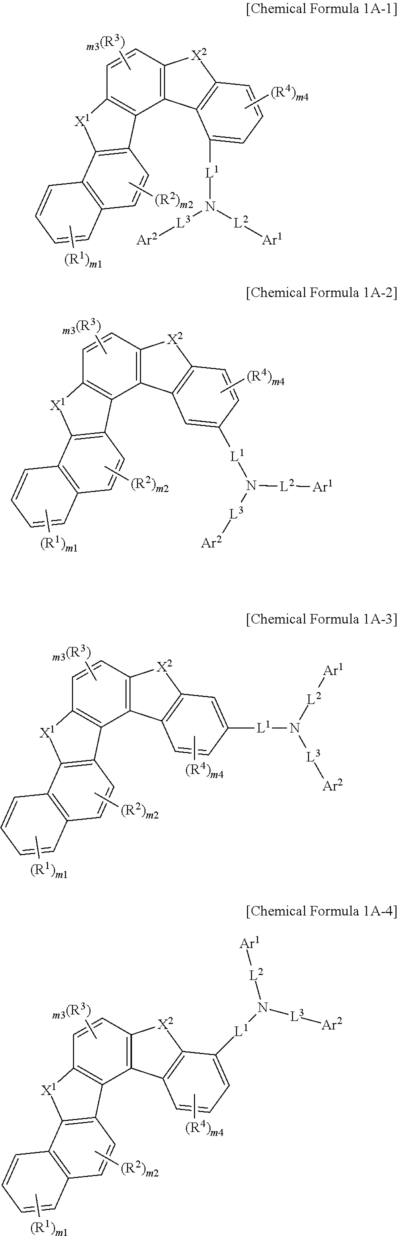

In Chemical Formula 1A-1, Chemical Formula 1A-2, Chemical Formula 1A-3, and Chemical Formula 1A-4, X¹, X², L¹ to L³, Ar¹, Ar², R¹ to R⁴, and m1 to m4 are the same as described above.

As a specific example, Chemical Formula 1B may be represented by one of Chemical Formula 1B-1, Chemical Formula 1B-2, Chemical Formula 1B-3, and Chemical Formula 1B-4, depending on the specific substitution position of the amine group.

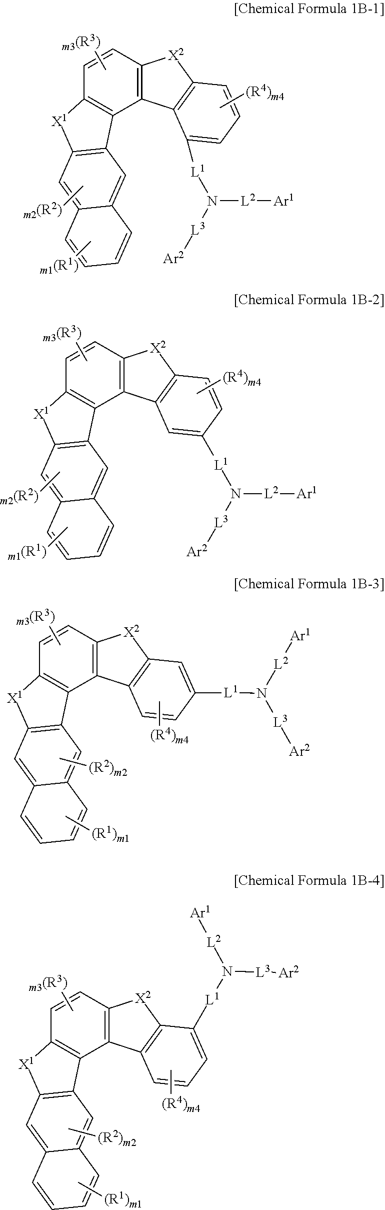

In Chemical Formula 1B-1, Chemical Formula 1B-2, Chemical Formula 1B-3, and Chemical Formula 1B-4, X¹, X², L¹ to L³, Ar¹, Ar², R¹ to R⁴, and m1 to m4 are the same as described above.

As a specific example, Chemical Formula 1C may be represented by one of Chemical Formula 1C-1, Chemical Formula 1C-2, Chemical Formula 1C-3, and Chemical Formula 1C-4, depending on the specific substitution position of the amine group.

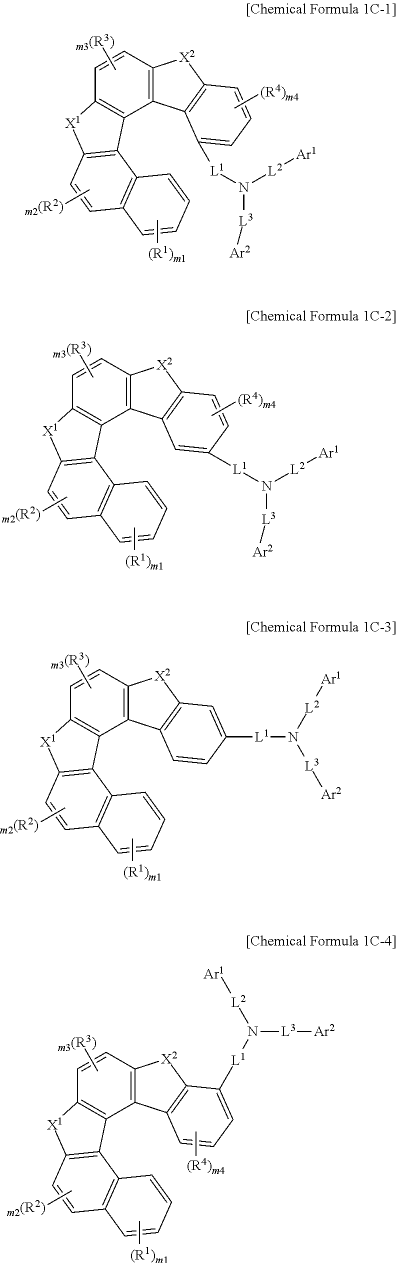

In Chemical Formula 1C-1 to Chemical Formula 1C-4, X¹, X², L¹ to L³, Ar¹, Ar², R¹ to R⁴, and m1 to m4 are the same as described above.

For example, Chemical Formula 1 may be represented by any one of Chemical Formula 1A-1 to Chemical Formula 1A-4.

For example, Ar¹ and Ar² may each independently be a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted chrysenyl group, a substituted or unsubstituted fluorenyl group, a substituted or unsubstituted triphenylene group, a substituted or unsubstituted dibenzofuranyl group, a substituted or unsubstituted dibenzothiophenyl group, a substituted or unsubstituted benzonaphthofuranyl group, a substituted or unsubstituted benzonaphthothiophenyl group, or a substituted or unsubstituted dibenzosilolyl group.

As a specific example, Ar¹ and Ar² may each independently be a substituted or unsubstituted phenyl group, a substituted or unsubstituted biphenyl group, a substituted or unsubstituted naphthyl group, a substituted or unsubstituted fluorenyl group, a substituted or unsubstituted dibenzofuranyl group, a substituted or unsubstituted dibenzothiophenyl group, or a substituted or unsubstituted dibenzosilolyl group.

For example, Ar¹ and Ar² may each independently be any one of the substituents listed in Group I.

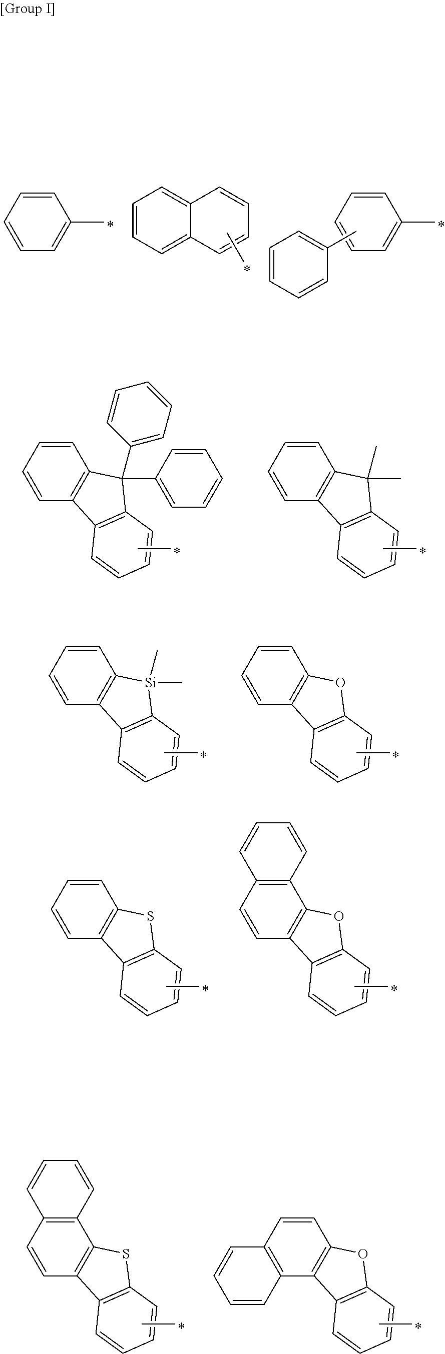

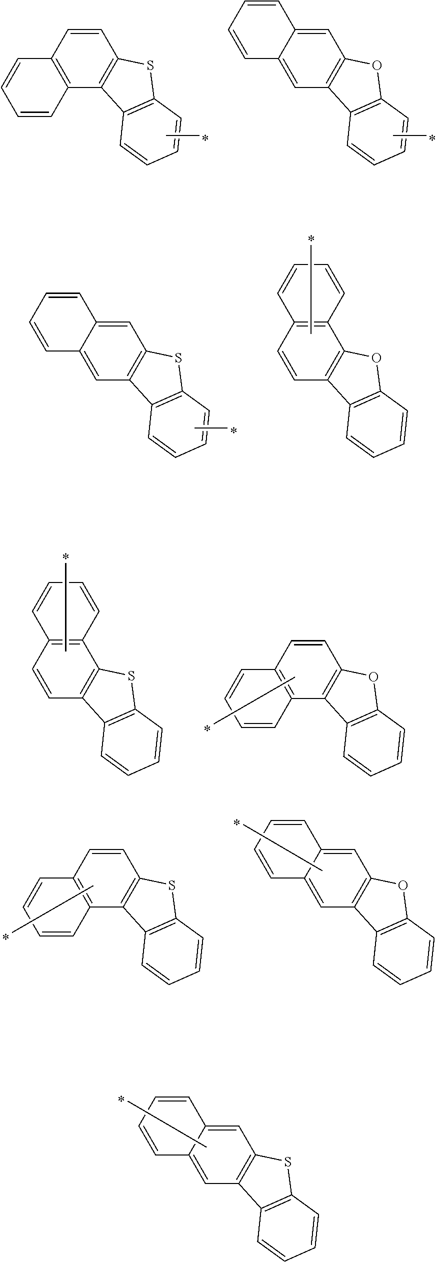

The substituents listed in Group I may be substituted with an additional substituent, and
the additional substituent may be, for example, deuterium, a substituted or unsubstituted C1 to C5 alkyl group or a substituted or unsubstituted C6 to C12 aryl group.

For example, L¹ may be a single bond, and
L² and L³ may each independently be a single bond or a substituted or unsubstituted phenylene group.

For example, R¹ to R⁴ may each independently be hydrogen, deuterium, a substituted or unsubstituted C1 to C5 alkyl group, or a substituted or unsubstituted C6 to C12 aryl group. As a specific example, R¹ to R⁴ may each be hydrogen or deuterium.

For example, X¹ may be O or S, and X² may be S.

For example, the compound for an organic optoelectronic device represented by

Chemical Formula 1 may include, but is not limited to, the compounds listed in Group 1.

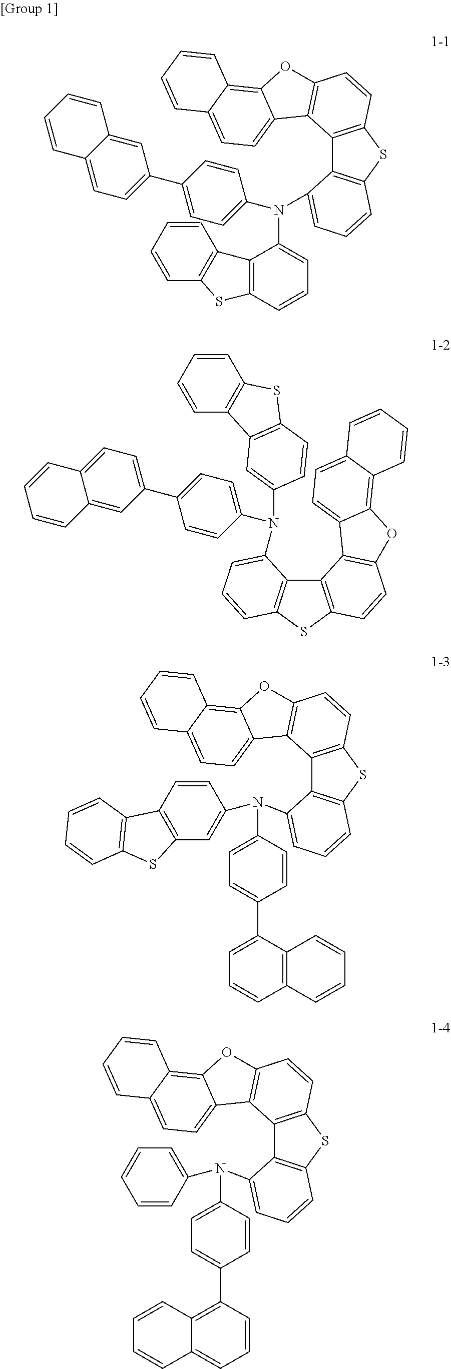 [SMILES: c1ccc2cc(-c3ccc(N(c4cccc5sc6ccccc6c45)c4cccc5sc6ccc7oc8c9ccccc9ccc8c7c6c45)cc3)ccc2c1 ; c1ccc2cc(-c3ccc(N(c4ccc5sc6ccccc6c5c4)c4cccc5sc6ccc7oc8c9ccccc9ccc8c7c6c45)cc3)ccc2c1 ; c1ccc2c(-c3ccc(N(c4ccc5c(c4)sc4ccccc45)c4cccc5sc6ccc7oc8c9ccccc9ccc8c7c6c45)cc3)cccc2c1 ; c1ccc(N(c2ccc(-c3cccc4ccccc34)cc2)c2cccc3sc4ccc5oc6c7ccccc7ccc6c5c4c23)cc1]

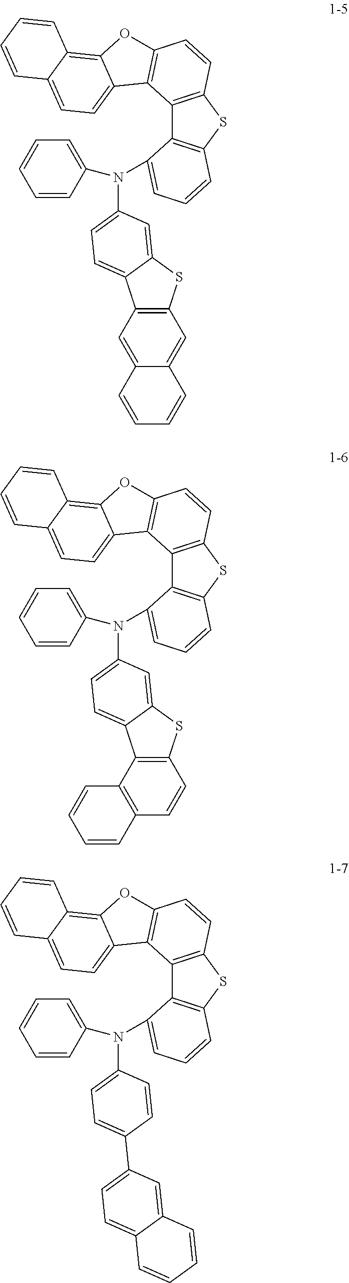 [SMILES: c1ccc(N(c2ccc3c(c2)sc2cc4ccccc4cc23)c2cccc3sc4ccc5oc6c7ccccc7ccc6c5c4c23)cc1 ; c1ccc(N(c2ccc3c(c2)sc2ccc4ccccc4c23)c2cccc3sc4ccc5oc6c7ccccc7ccc6c5c4c23)cc1 ; c1ccc(N(c2ccc(-c3ccc4ccccc4c3)cc2)c2cccc3sc4ccc5oc6c7ccccc7ccc6c5c4c23)cc1]

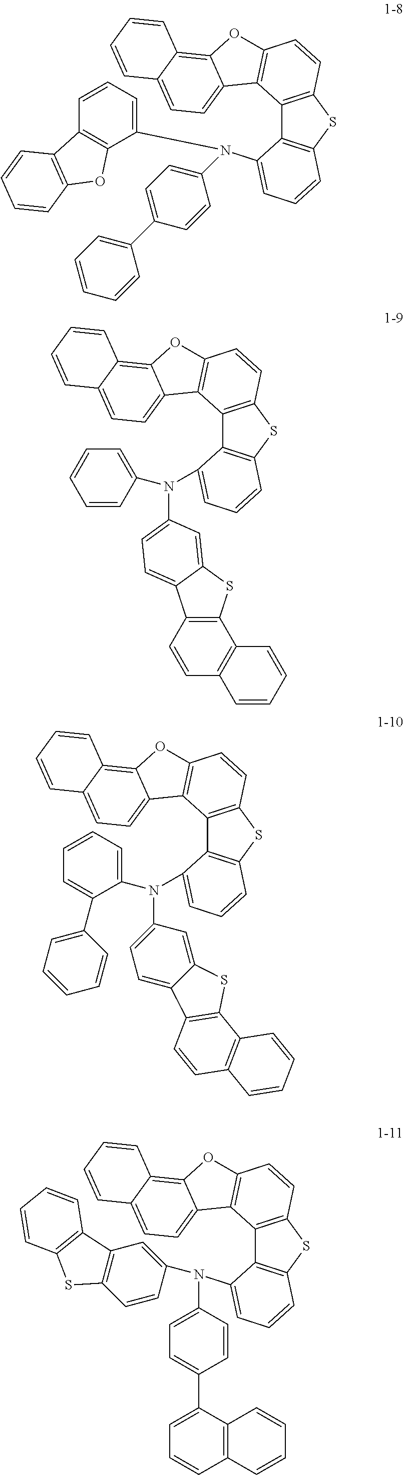

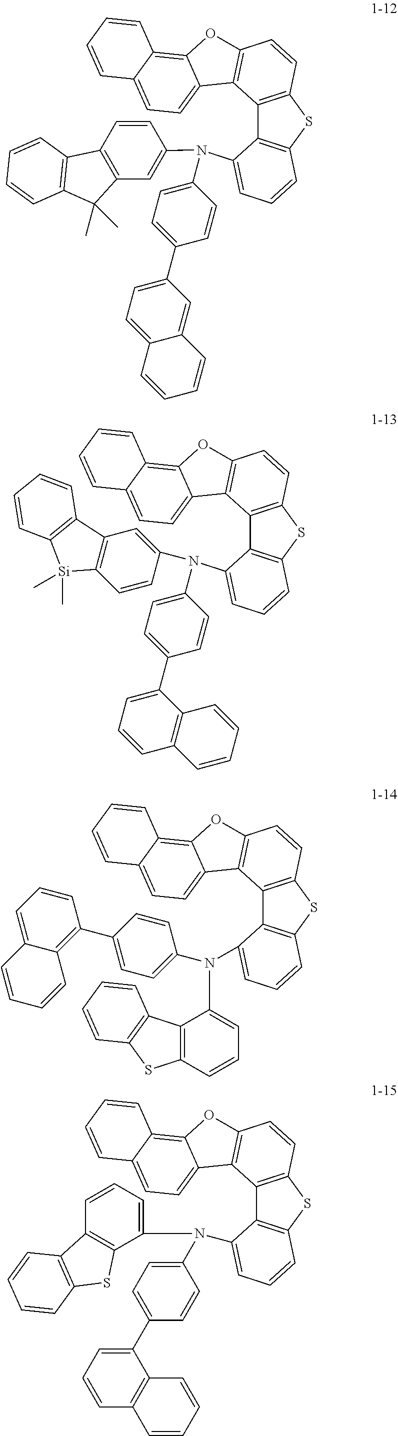

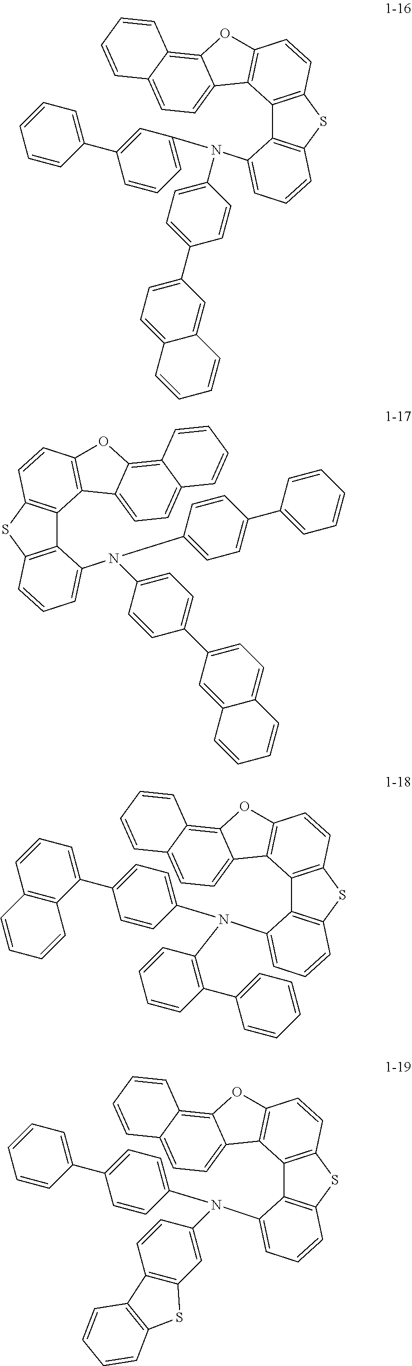

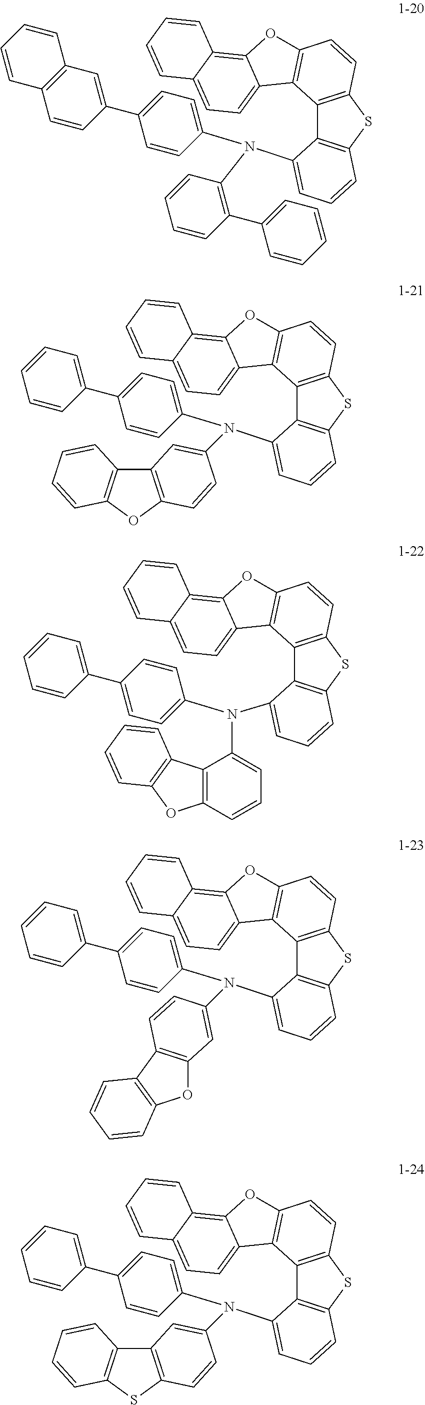

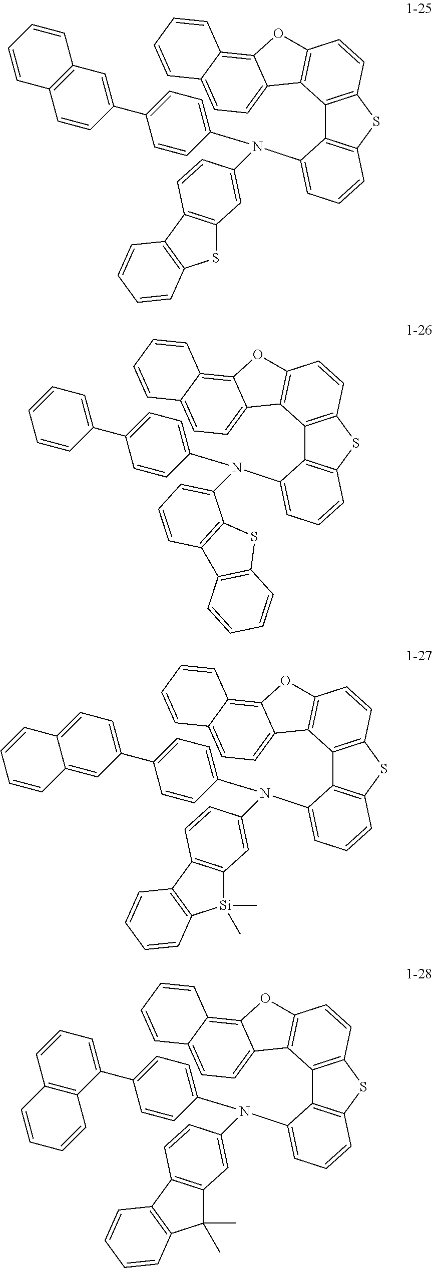

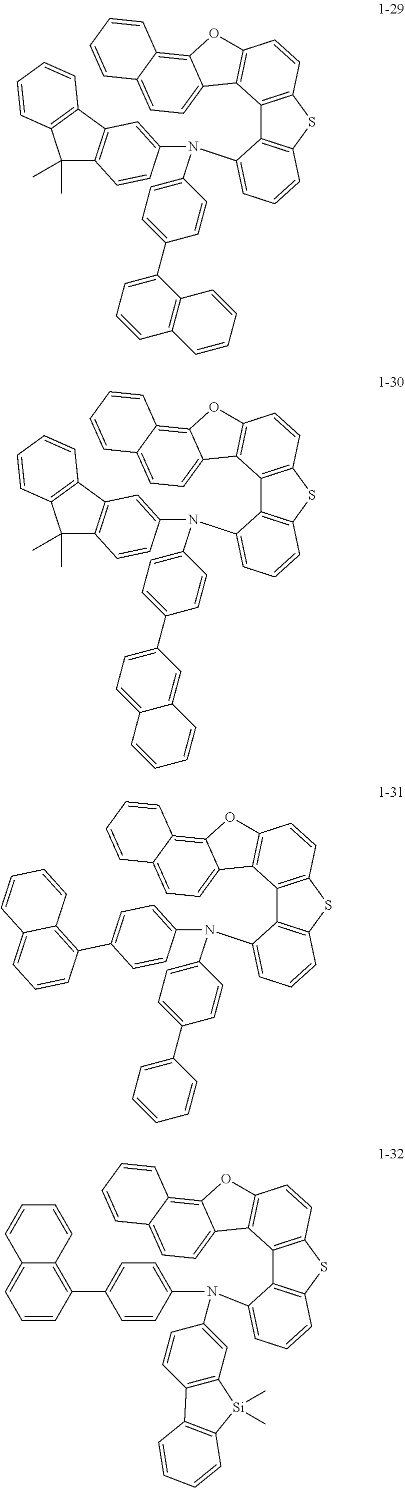

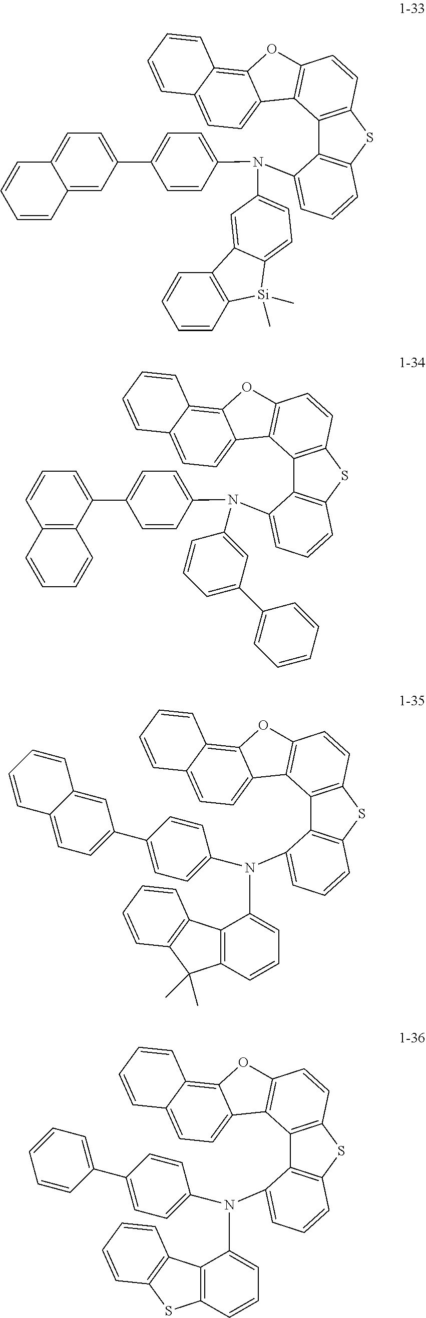

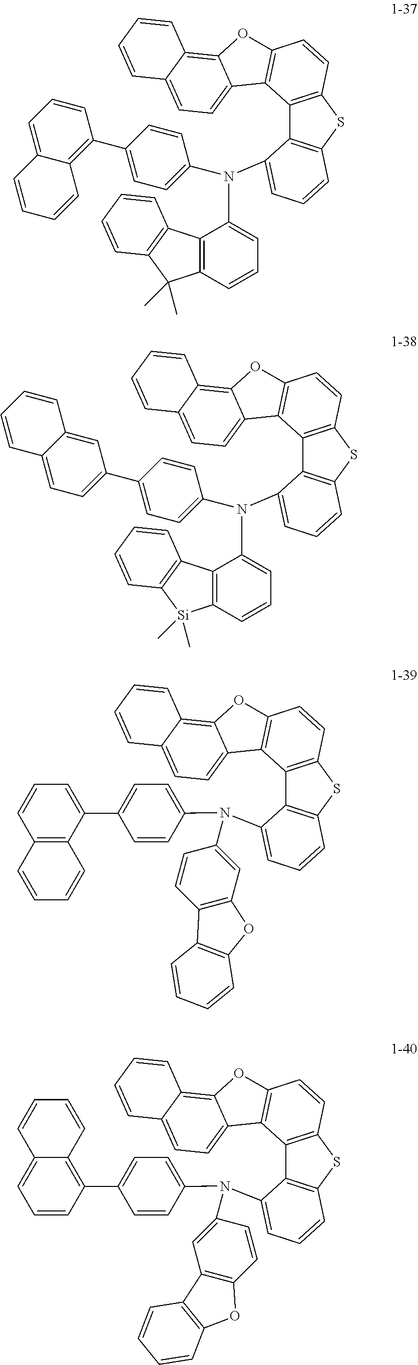

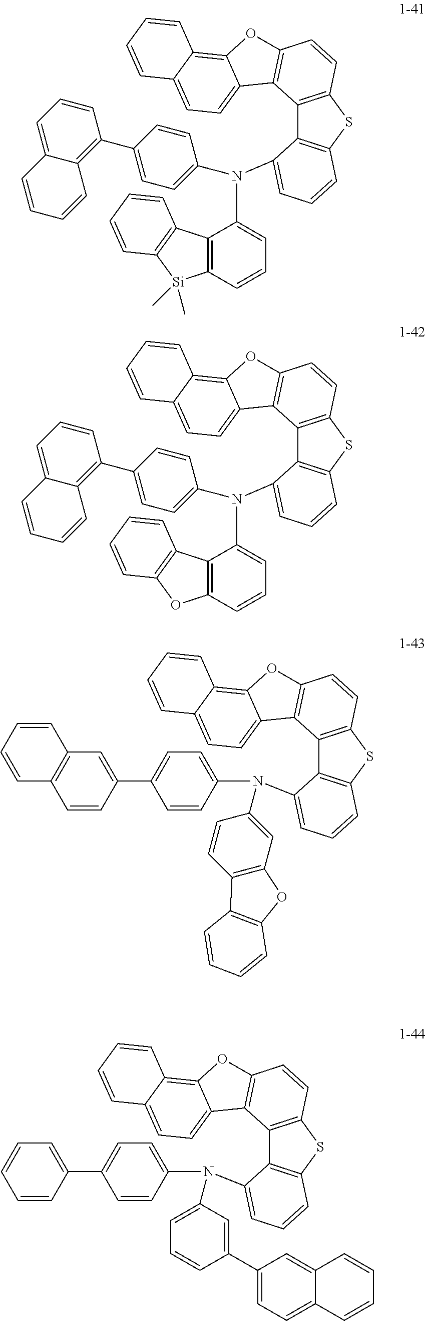

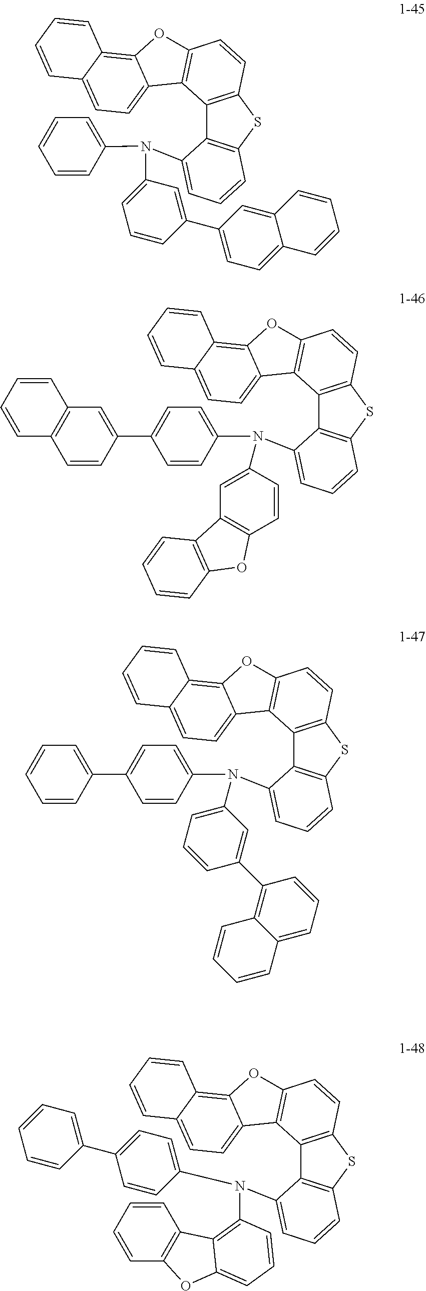 [SMILES: c1ccc(N(c2cccc(-c3ccc4ccccc4c3)c2)c2cccc3sc4ccc5oc6c7ccccc7ccc6c5c4c23)cc1 ; c1ccc2cc(-c3ccc(N(c4ccc5oc6ccccc6c5c4)c4cccc5sc6ccc7oc8c9ccccc9ccc8c7c6c45)cc3)ccc2c1 ; c1ccc(-c2ccc(N(c3cccc(-c4cccc5ccccc45)c3)c3cccc4sc5ccc6oc7c8ccccc8ccc7c6c5c34)cc2)cc1 ; c1ccc(-c2ccc(N(c3cccc4oc5ccccc5c34)c3cccc4sc5ccc6oc7c8ccccc8ccc7c6c5c34)cc2)cc1]

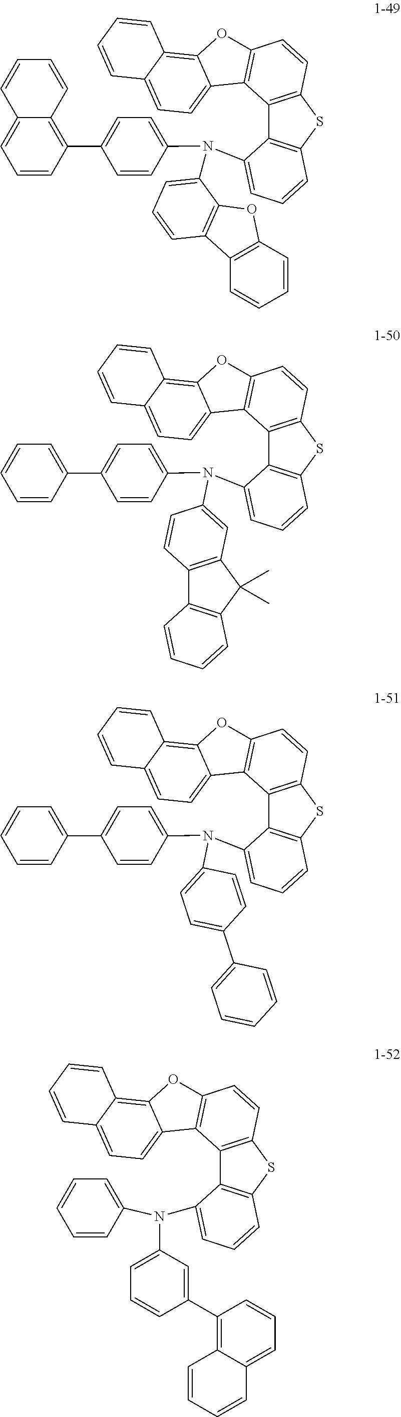 [SMILES: c1ccc2c(-c3ccc(N(c4cccc5c4oc4ccccc45)c4cccc5sc6ccc7oc8c9ccccc9ccc8c7c6c45)cc3)cccc2c1 ; CC1(C)c2ccccc2-c2ccc(N(c3ccc(-c4ccccc4)cc3)c3cccc4sc5ccc6oc7c8ccccc8ccc7c6c5c34)cc21 ; c1ccc(-c2ccc(N(c3ccc(-c4ccccc4)cc3)c3cccc4sc5ccc6oc7c8ccccc8ccc7c6c5c34)cc2)cc1 ; c1ccc(N(c2cccc(-c3cccc4ccccc34)c2)c2cccc3sc4ccc5oc6c7ccccc7ccc6c5c4c23)cc1]

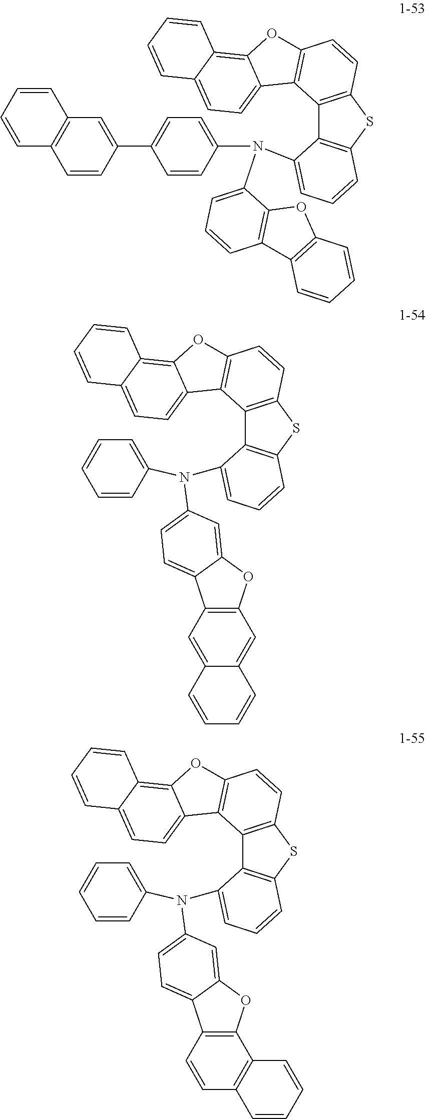

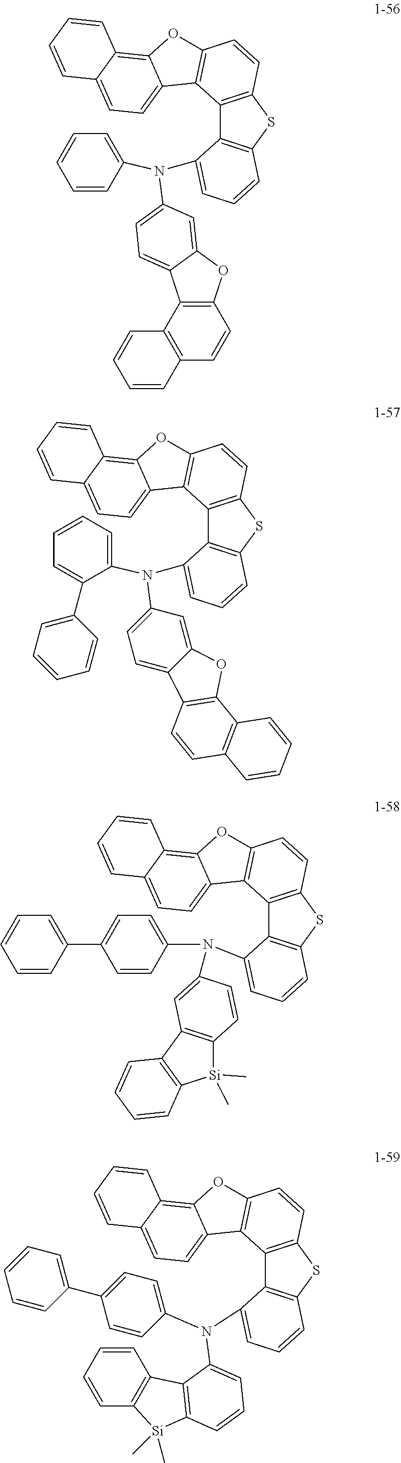 [SMILES: c1ccc(N(c2ccc3c(c2)oc2ccc4ccccc4c23)c2cccc3sc4ccc5oc6c7ccccc7ccc6c5c4c23)cc1 ; c1ccc(-c2ccccc2N(c2ccc3c(c2)oc2c4ccccc4ccc32)c2cccc3sc4ccc5oc6c7ccccc7ccc6c5c4c23)cc1 ; C[Si]1(C)c2ccccc2-c2cc(N(c3ccc(-c4ccccc4)cc3)c3cccc4sc5ccc6oc7c8ccccc8ccc7c6c5c34)ccc21 ; C[Si]1(C)c2ccccc2-c2c(N(c3ccc(-c4ccccc4)cc3)c3cccc4sc5ccc6oc7c8ccccc8ccc7c6c5c34)cccc21]

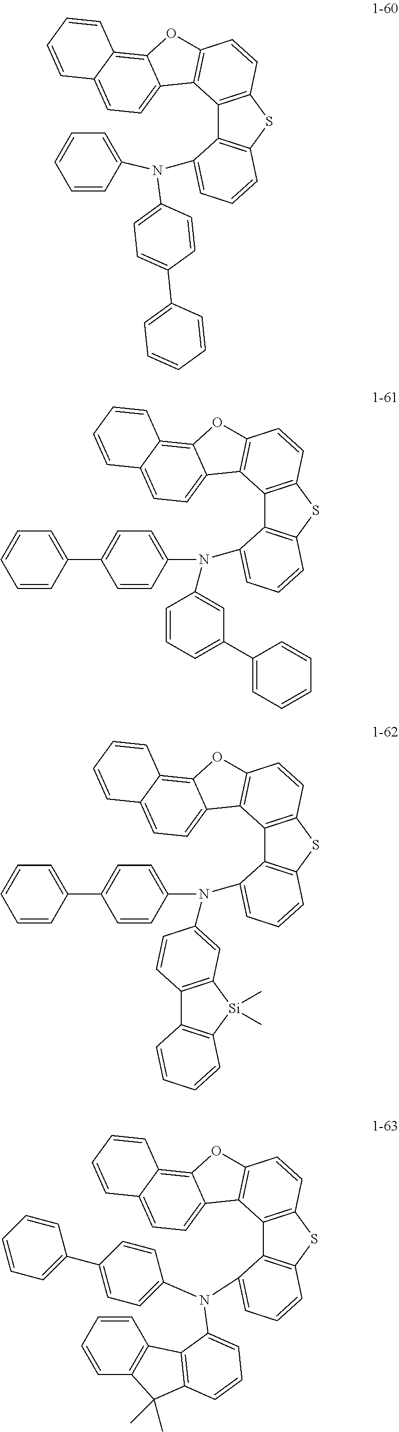 [SMILES: c1ccc(-c2ccc(N(c3ccccc3)c3cccc4sc5ccc6oc7c8ccccc8ccc7c6c5c34)cc2)cc1 ; c1ccc(-c2ccc(N(c3cccc(-c4ccccc4)c3)c3cccc4sc5ccc6oc7c8ccccc8ccc7c6c5c34)cc2)cc1 ; C[Si]1(C)c2ccccc2-c2ccc(N(c3ccc(-c4ccccc4)cc3)c3cccc4sc5ccc6oc7c8ccccc8ccc7c6c5c34)cc21 ; CC1(C)c2ccccc2-c2c(N(c3ccc(-c4ccccc4)cc3)c3cccc4sc5ccc6oc7c8ccccc8ccc7c6c5c34)cccc21]

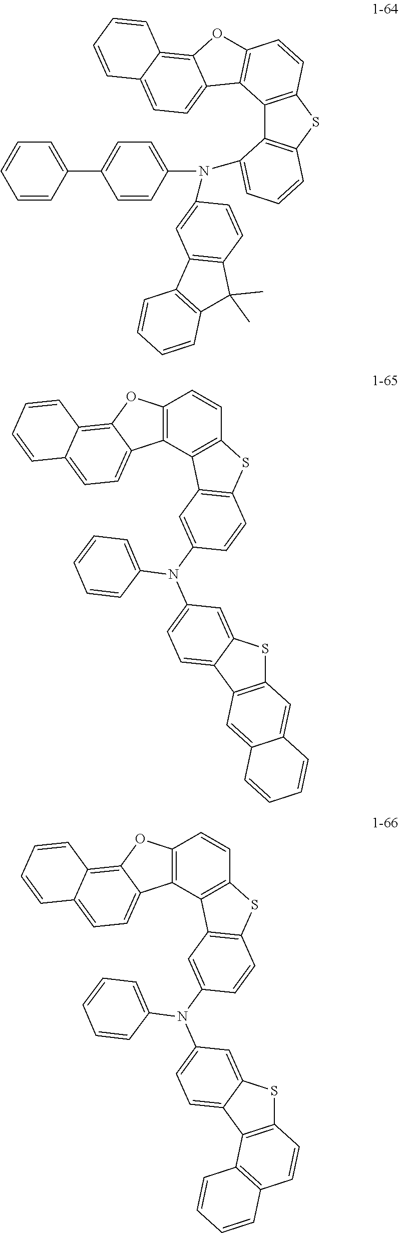

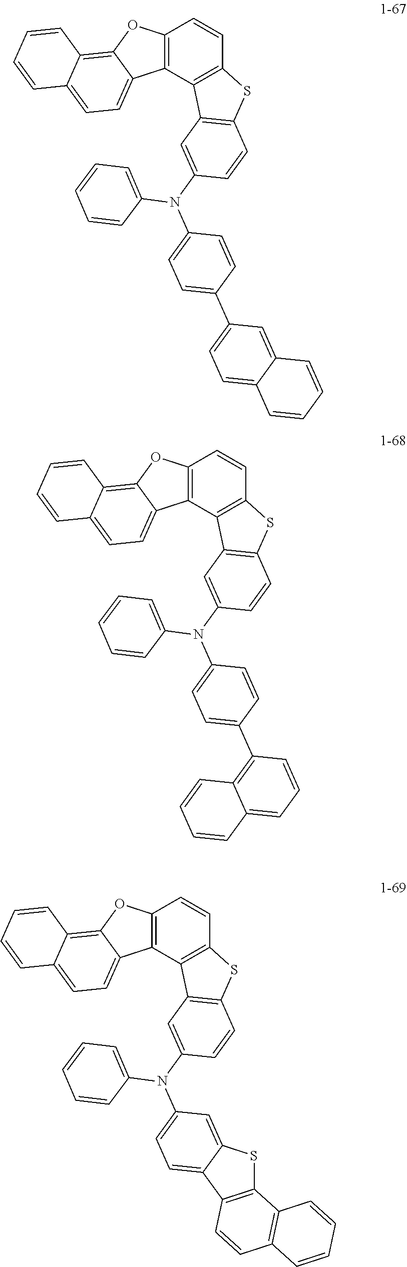 [SMILES: c1ccc(N(c2ccc(-c3ccc4ccccc4c3)cc2)c2ccc3sc4ccc5oc6c7ccccc7ccc6c5c4c3c2)cc1 ; c1ccc(N(c2ccc(-c3cccc4ccccc34)cc2)c2ccc3sc4ccc5oc6c7ccccc7ccc6c5c4c3c2)cc1 ; c1ccc(N(c2ccc3c(c2)sc2c4ccccc4ccc32)c2ccc3sc4ccc5oc6c7ccccc7ccc6c5c4c3c2)cc1]

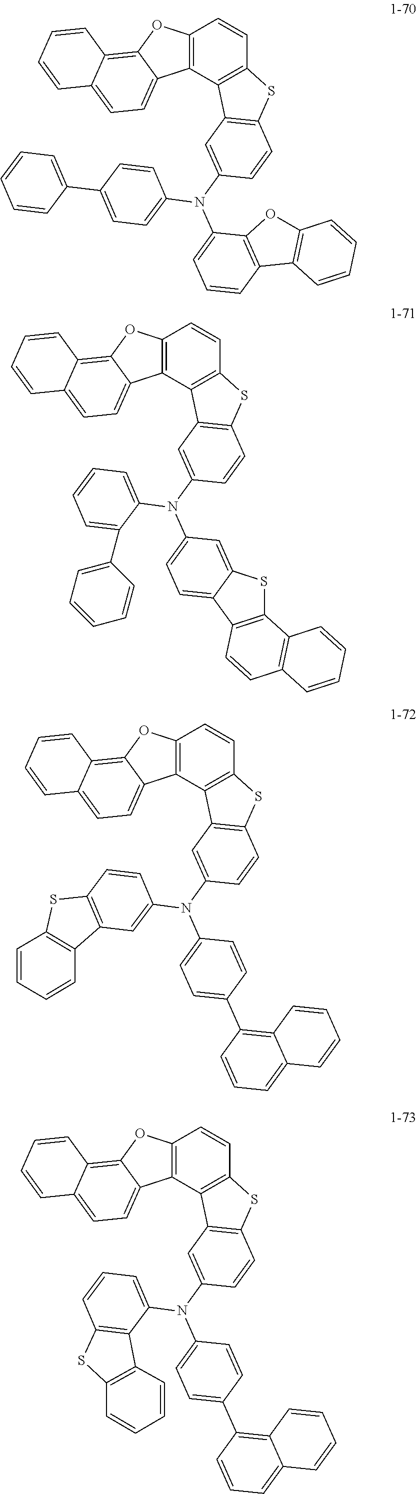

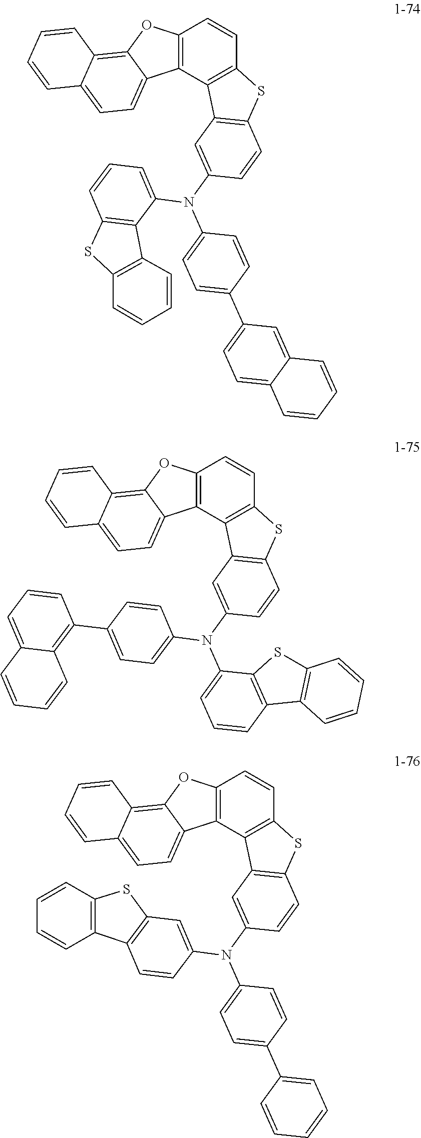

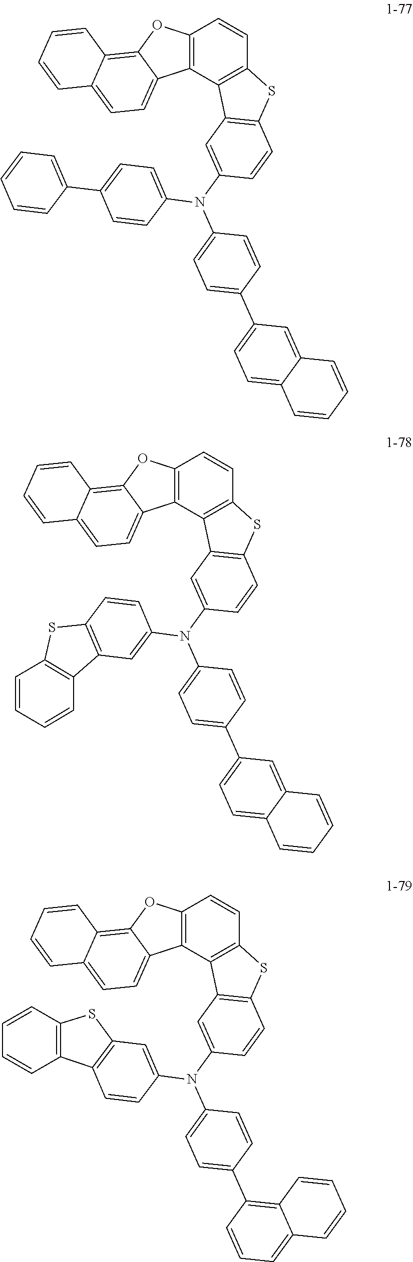 [SMILES: c1ccc(-c2ccc(N(c3ccc(-c4ccc5ccccc5c4)cc3)c3ccc4sc5ccc6oc7c8ccccc8ccc7c6c5c4c3)cc2)cc1 ; c1ccc2cc(-c3ccc(N(c4ccc5sc6ccccc6c5c4)c4ccc5sc6ccc7oc8c9ccccc9ccc8c7c6c5c4)cc3)ccc2c1 ; c1ccc2c(-c3ccc(N(c4ccc5c(c4)sc4ccccc45)c4ccc5sc6ccc7oc8c9ccccc9ccc8c7c6c5c4)cc3)cccc2c1]

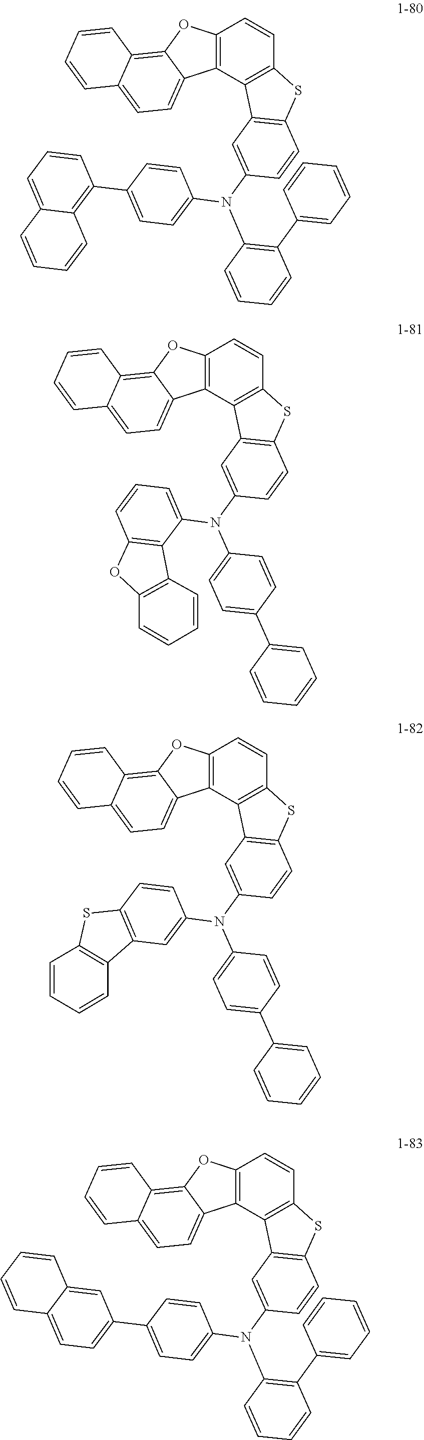

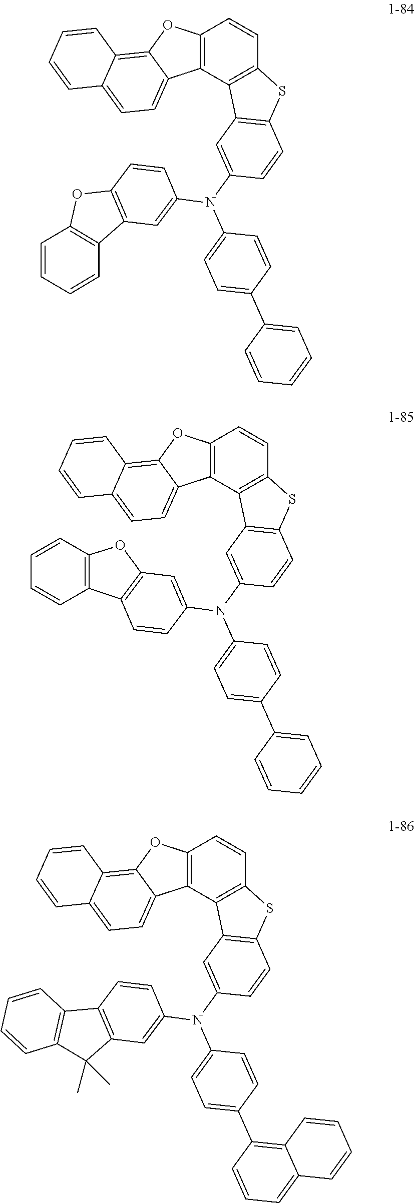 [SMILES: c1ccc(-c2ccc(N(c3ccc4oc5ccccc5c4c3)c3ccc4sc5ccc6oc7c8ccccc8ccc7c6c5c4c3)cc2)cc1 ; c1ccc(-c2ccc(N(c3ccc4c(c3)oc3ccccc34)c3ccc4sc5ccc6oc7c8ccccc8ccc7c6c5c4c3)cc2)cc1 ; CC1(C)c2ccccc2-c2ccc(N(c3ccc(-c4cccc5ccccc45)cc3)c3ccc4sc5ccc6oc7c8ccccc8ccc7c6c5c4c3)cc21]

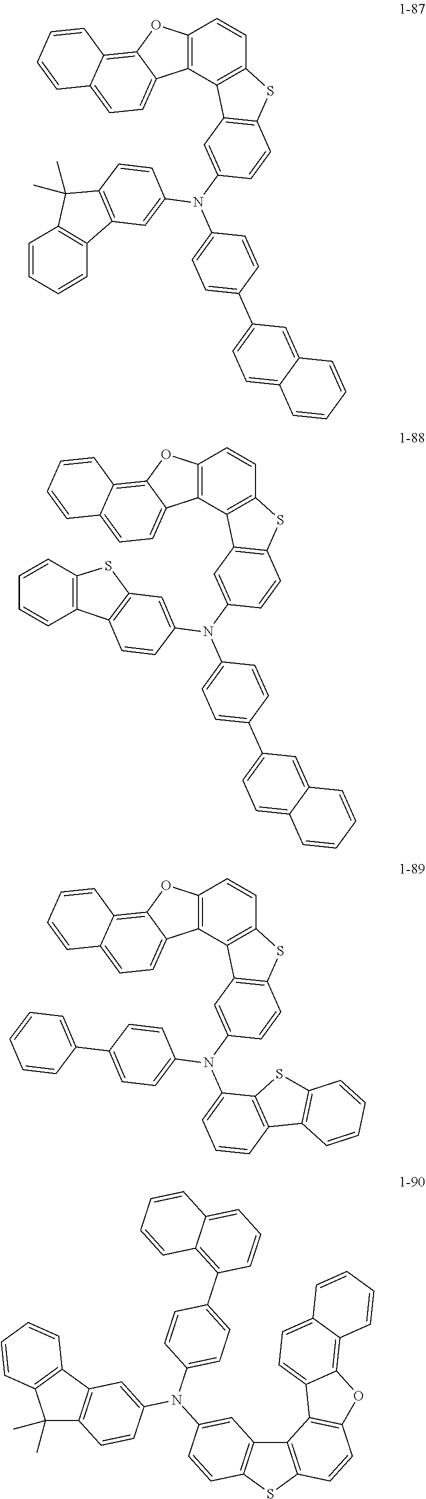 [SMILES: CC1(C)c2ccccc2-c2cc(N(c3ccc(-c4ccc5ccccc5c4)cc3)c3ccc4sc5ccc6oc7c8ccccc8ccc7c6c5c4c3)ccc21 ; c1ccc2cc(-c3ccc(N(c4ccc5c(c4)sc4ccccc45)c4ccc5sc6ccc7oc8c9ccccc9ccc8c7c6c5c4)cc3)ccc2c1 ; c1ccc(-c2ccc(N(c3ccc4sc5ccc6oc7c8ccccc8ccc7c6c5c4c3)c3cccc4c3sc3ccccc34)cc2)cc1 ; CC1(C)c2ccccc2-c2cc(N(c3ccc(-c4cccc5ccccc45)cc3)c3ccc4sc5ccc6oc7c8ccccc8ccc7c6c5c4c3)ccc21]

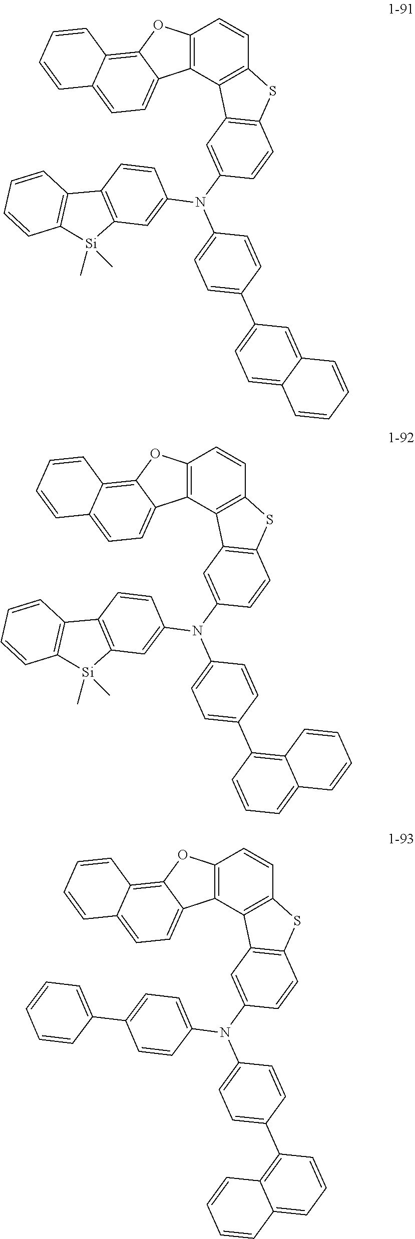

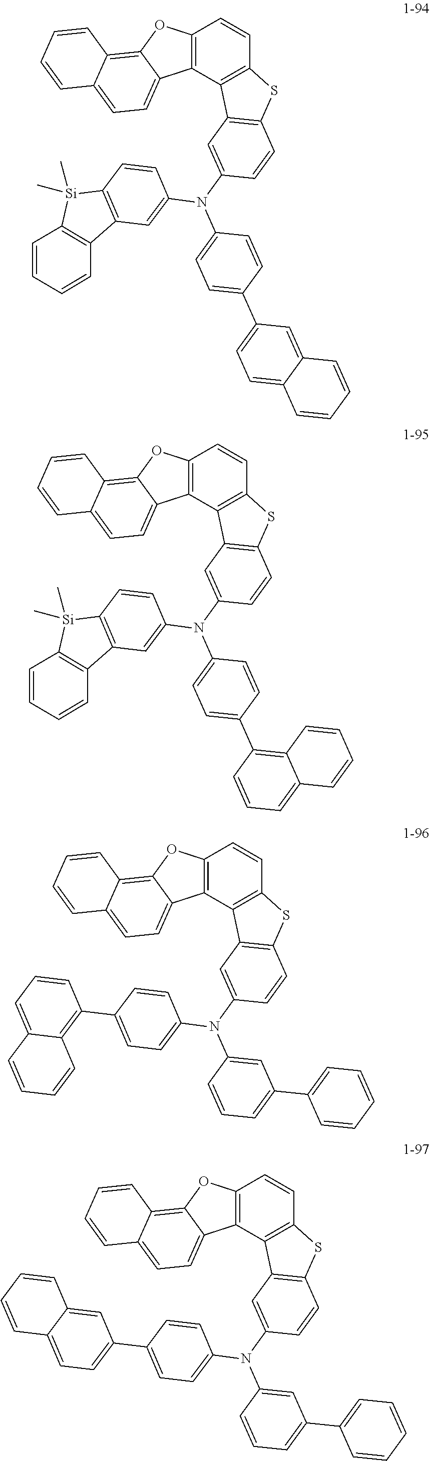

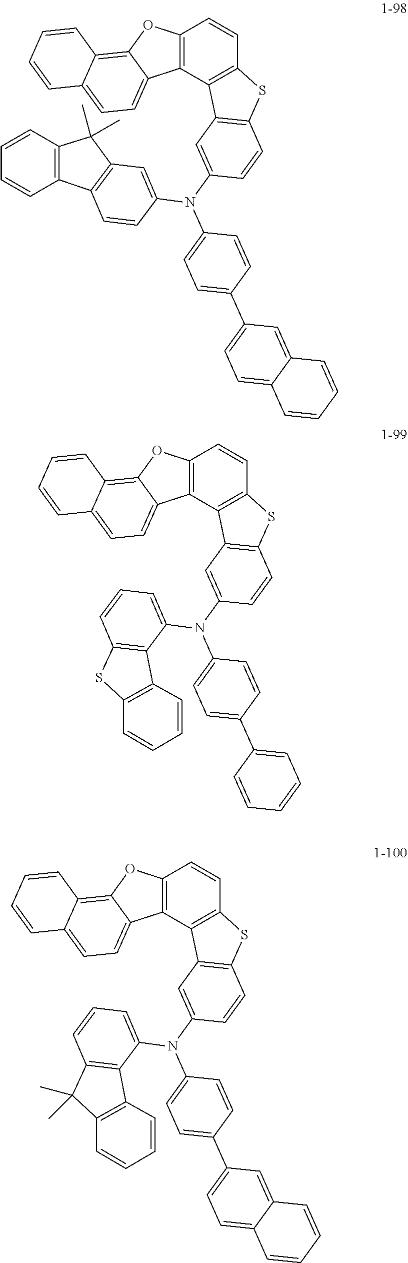 [SMILES: CC1(C)c2ccccc2-c2ccc(N(c3ccc(-c4ccc5ccccc5c4)cc3)c3ccc4sc5ccc6oc7c8ccccc8ccc7c6c5c4c3)cc21 ; c1ccc(-c2ccc(N(c3ccc4sc5ccc6oc7c8ccccc8ccc7c6c5c4c3)c3cccc4sc5ccccc5c34)cc2)cc1 ; CC1(C)c2ccccc2-c2c(N(c3ccc(-c4ccc5ccccc5c4)cc3)c3ccc4sc5ccc6oc7c8ccccc8ccc7c6c5c4c3)cccc21]

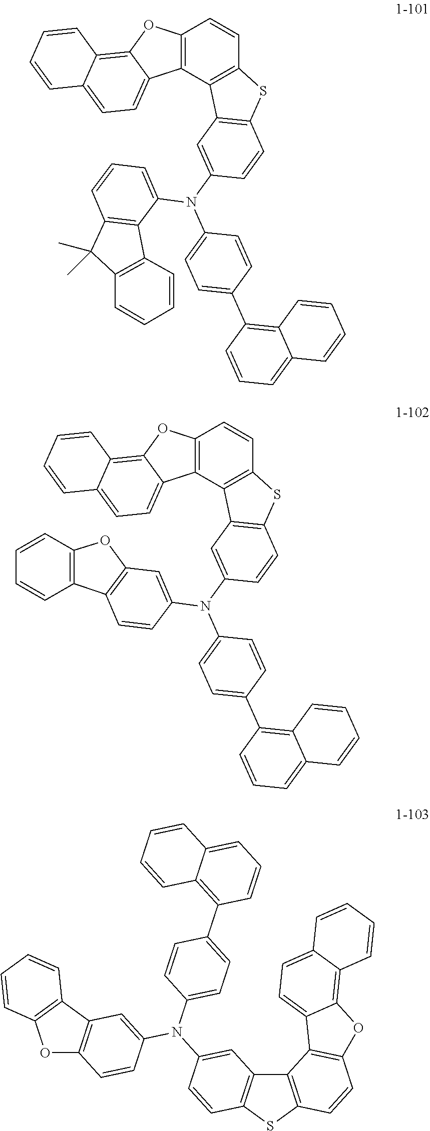

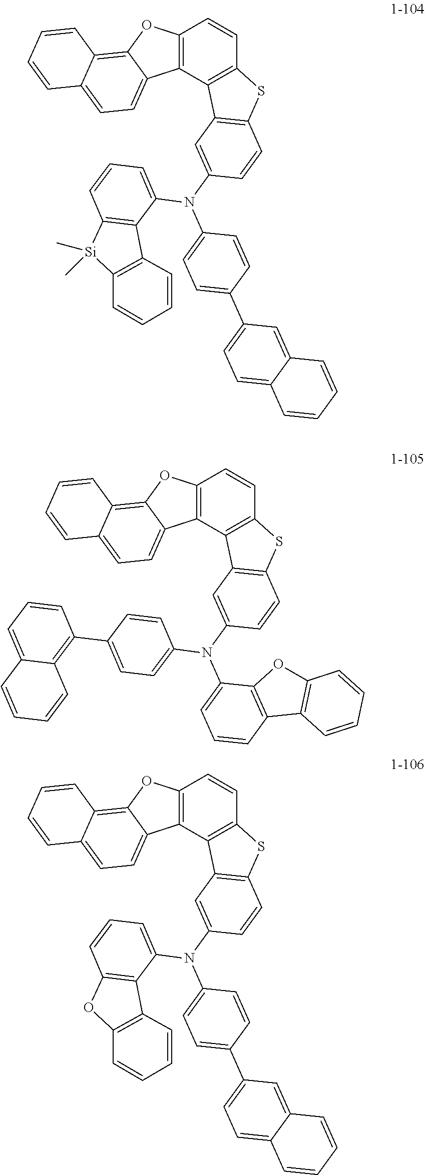

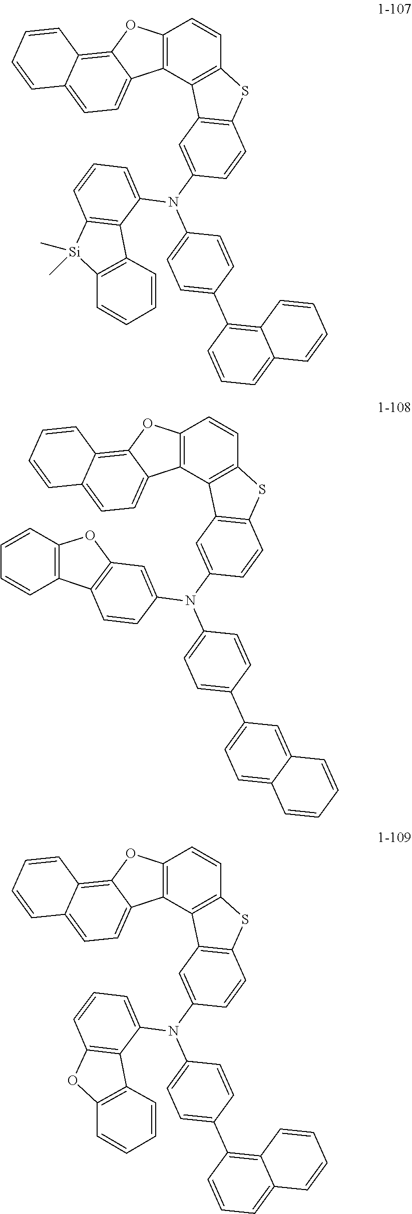

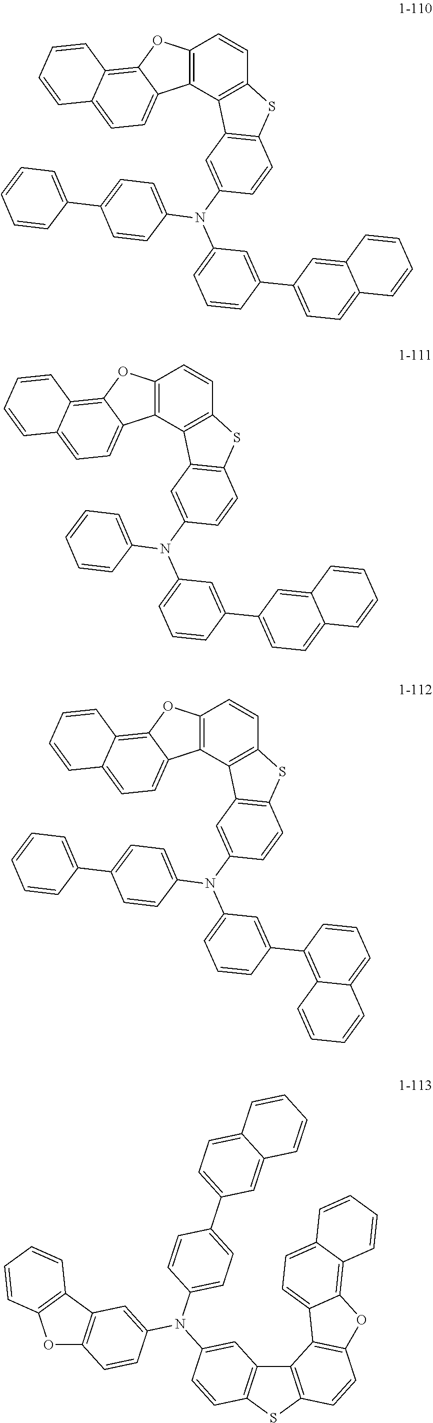 [SMILES: c1ccc(-c2ccc(N(c3cccc(-c4ccc5ccccc5c4)c3)c3ccc4sc5ccc6oc7c8ccccc8ccc7c6c5c4c3)cc2)cc1 ; c1ccc(N(c2cccc(-c3ccc4ccccc4c3)c2)c2ccc3sc4ccc5oc6c7ccccc7ccc6c5c4c3c2)cc1 ; c1ccc(-c2ccc(N(c3cccc(-c4cccc5ccccc45)c3)c3ccc4sc5ccc6oc7c8ccccc8ccc7c6c5c4c3)cc2)cc1 ; c1ccc2cc(-c3ccc(N(c4ccc5oc6ccccc6c5c4)c4ccc5sc6ccc7oc8c9ccccc9ccc8c7c6c5c4)cc3)ccc2c1]

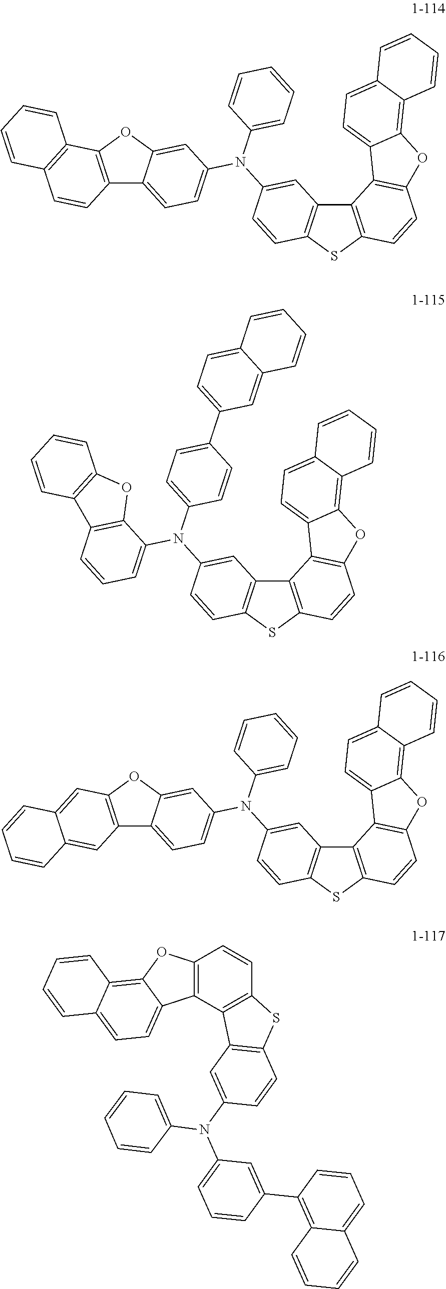 [SMILES: c1ccc(N(c2ccc3c(c2)oc2c4ccccc4ccc32)c2ccc3sc4ccc5oc6c7ccccc7ccc6c5c4c3c2)cc1 ; c1ccc2cc(-c3ccc(N(c4ccc5sc6ccc7oc8c9ccccc9ccc8c7c6c5c4)c4cccc5c4oc4ccccc45)cc3)ccc2c1 ; c1ccc(N(c2ccc3c(c2)oc2cc4ccccc4cc23)c2ccc3sc4ccc5oc6c7ccccc7ccc6c5c4c3c2)cc1 ; c1ccc(N(c2cccc(-c3cccc4ccccc34)c2)c2ccc3sc4ccc5oc6c7ccccc7ccc6c5c4c3c2)cc1]

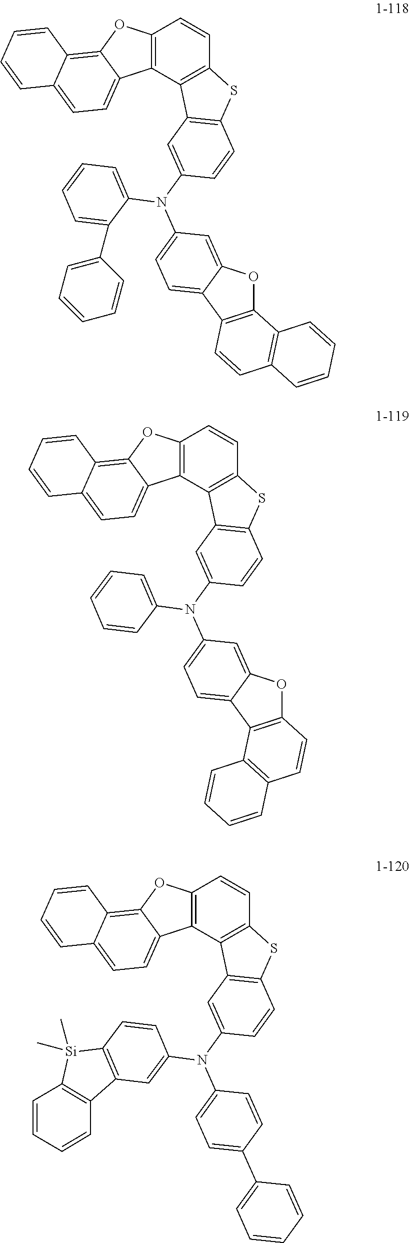 [SMILES: c1ccc(-c2ccccc2N(c2ccc3c(c2)oc2c4ccccc4ccc32)c2ccc3sc4ccc5oc6c7ccccc7ccc6c5c4c3c2)cc1 ; c1ccc(N(c2ccc3c(c2)oc2ccc4ccccc4c23)c2ccc3sc4ccc5oc6c7ccccc7ccc6c5c4c3c2)cc1 ; C[Si]1(C)c2ccccc2-c2cc(N(c3ccc(-c4ccccc4)cc3)c3ccc4sc5ccc6oc7c8ccccc8ccc7c6c5c4c3)ccc21]

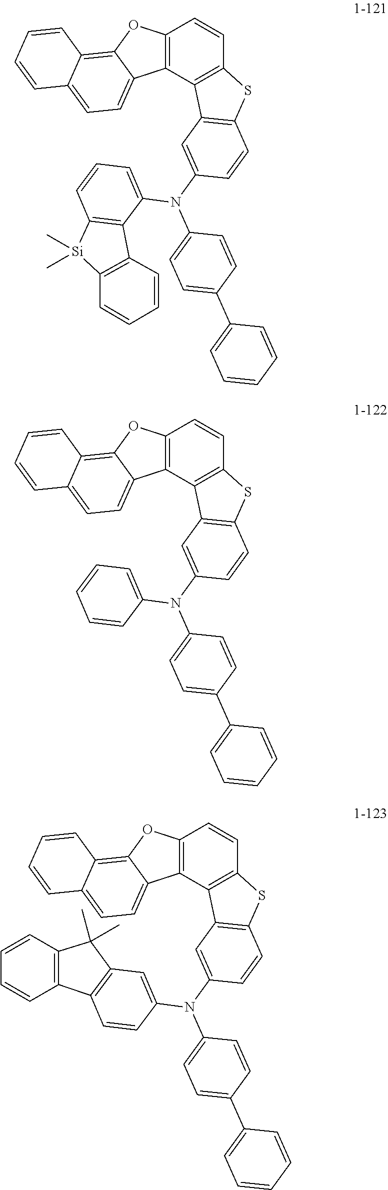

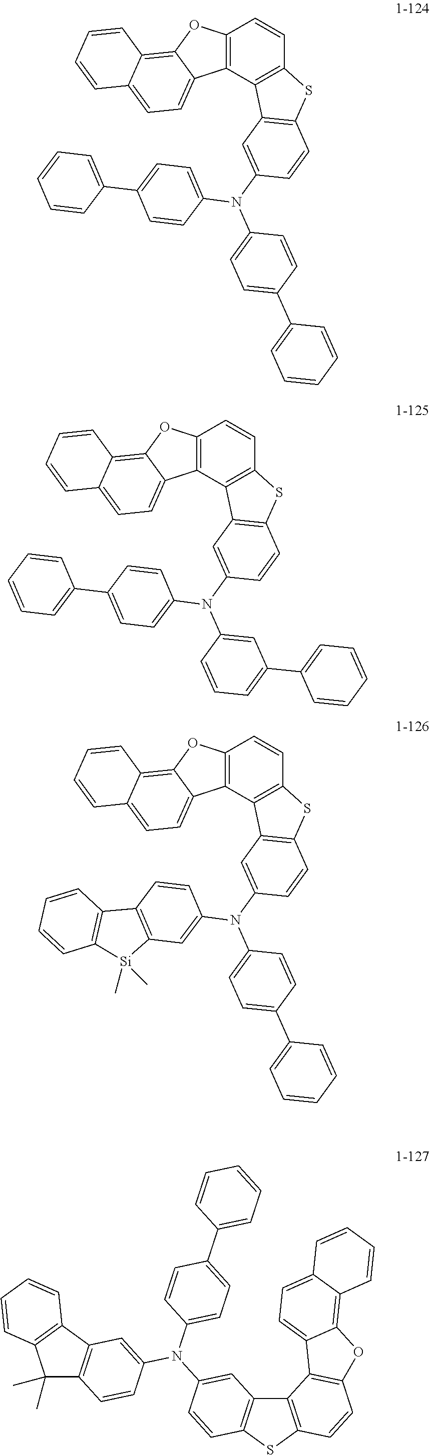

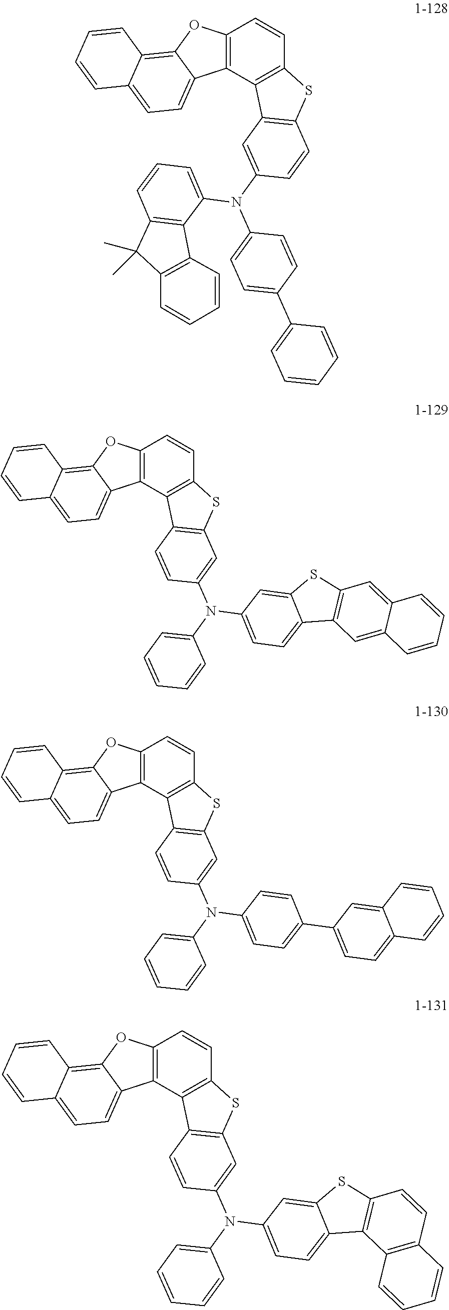

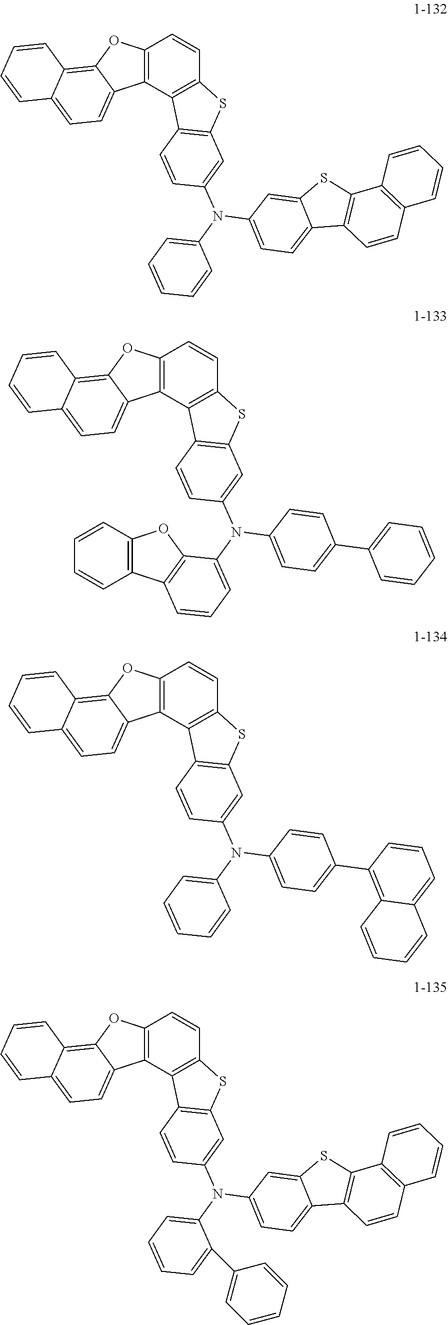 [SMILES: c1ccc(N(c2ccc3c(c2)sc2c4ccccc4ccc32)c2ccc3c(c2)sc2ccc4oc5c6ccccc6ccc5c4c23)cc1 ; c1ccc(-c2ccc(N(c3ccc4c(c3)sc3ccc5oc6c7ccccc7ccc6c5c34)c3cccc4c3oc3ccccc34)cc2)cc1 ; c1ccc(N(c2ccc(-c3cccc4ccccc34)cc2)c2ccc3c(c2)sc2ccc4oc5c6ccccc6ccc5c4c23)cc1 ; c1ccc(-c2ccccc2N(c2ccc3c(c2)sc2c4ccccc4ccc32)c2ccc3c(c2)sc2ccc4oc5c6ccccc6ccc5c4c23)cc1]

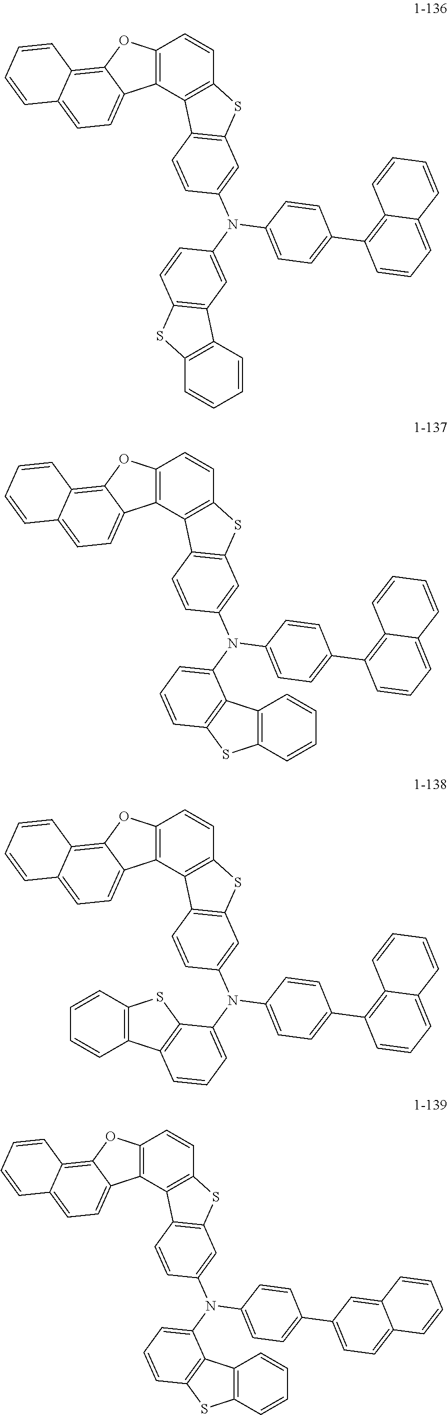 [SMILES: c1ccc2c(-c3ccc(N(c4ccc5c(c4)sc4ccc6oc7c8ccccc8ccc7c6c45)c4ccc5sc6ccccc6c5c4)cc3)cccc2c1 ; c1ccc2c(-c3ccc(N(c4ccc5c(c4)sc4ccc6oc7c8ccccc8ccc7c6c45)c4cccc5sc6ccccc6c45)cc3)cccc2c1 ; c1ccc2c(-c3ccc(N(c4ccc5c(c4)sc4ccc6oc7c8ccccc8ccc7c6c45)c4cccc5c4sc4ccccc45)cc3)cccc2c1 ; c1ccc2cc(-c3ccc(N(c4ccc5c(c4)sc4ccc6oc7c8ccccc8ccc7c6c45)c4cccc5sc6ccccc6c45)cc3)ccc2c1]

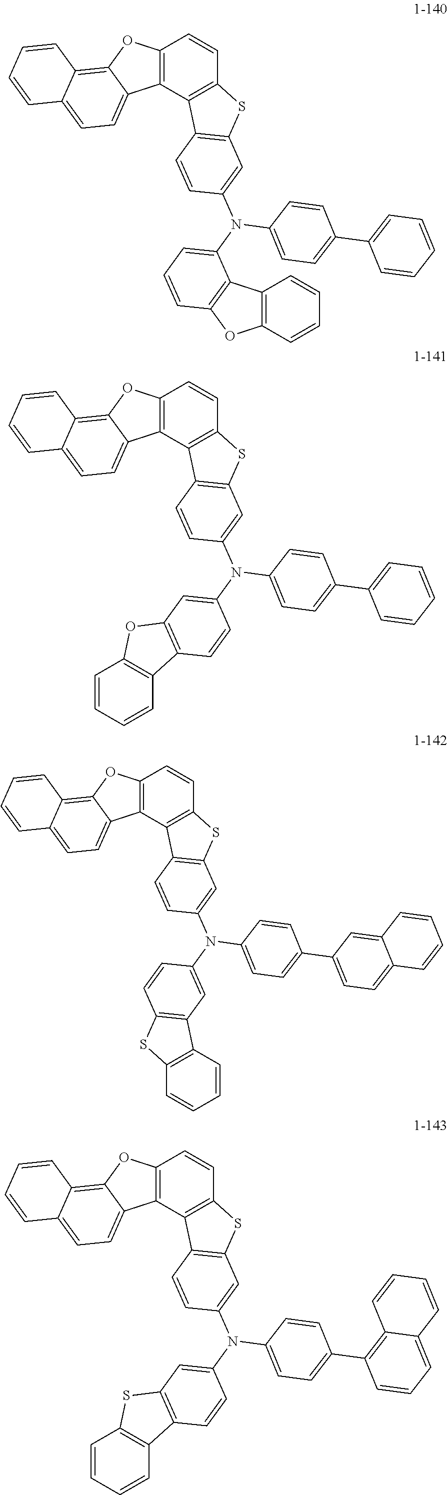

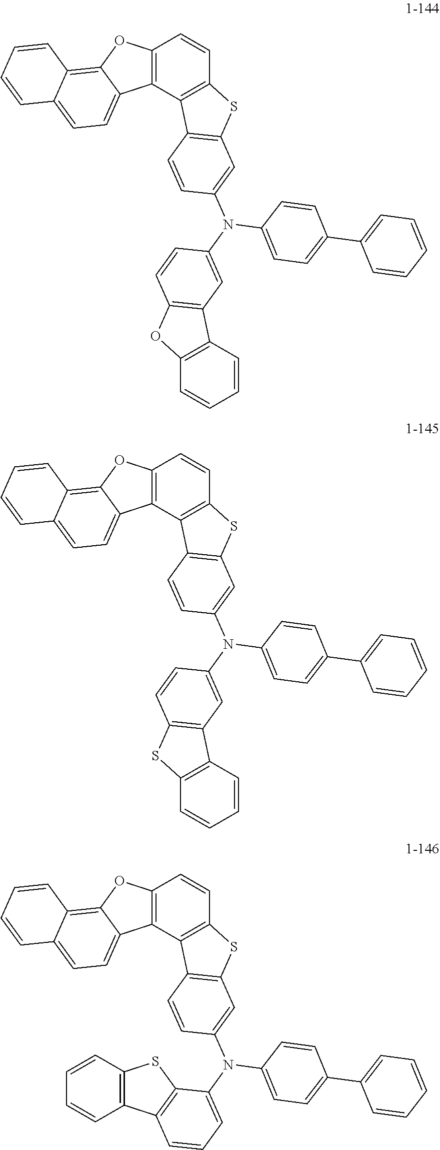 [SMILES: c1ccc(-c2ccc(N(c3ccc4c(c3)sc3ccc5oc6c7ccccc7ccc6c5c34)c3ccc4oc5ccccc5c4c3)cc2)cc1 ; c1ccc(-c2ccc(N(c3ccc4c(c3)sc3ccc5oc6c7ccccc7ccc6c5c34)c3ccc4sc5ccccc5c4c3)cc2)cc1 ; c1ccc(-c2ccc(N(c3ccc4c(c3)sc3ccc5oc6c7ccccc7ccc6c5c34)c3cccc4c3sc3ccccc34)cc2)cc1]

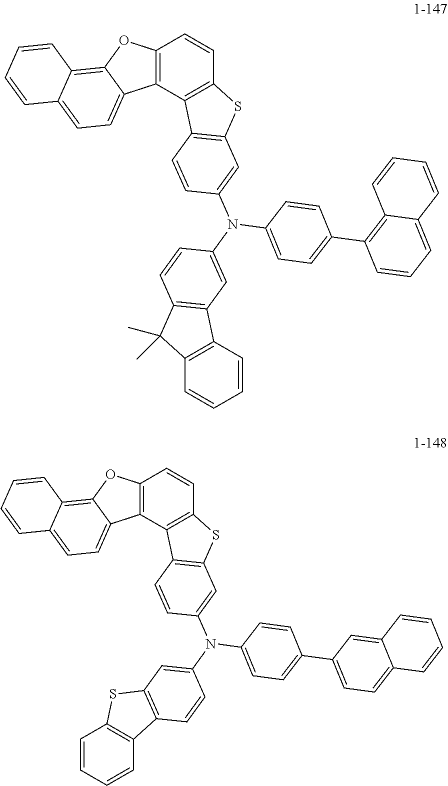 [SMILES: CC1(C)c2ccccc2-c2cc(N(c3ccc(-c4cccc5ccccc45)cc3)c3ccc4c(c3)sc3ccc5oc6c7ccccc7ccc6c5c34)ccc21 ; c1ccc2cc(-c3ccc(N(c4ccc5c(c4)sc4ccccc45)c4ccc5c(c4)sc4ccc6oc7c8ccccc8ccc7c6c45)cc3)ccc2c1]

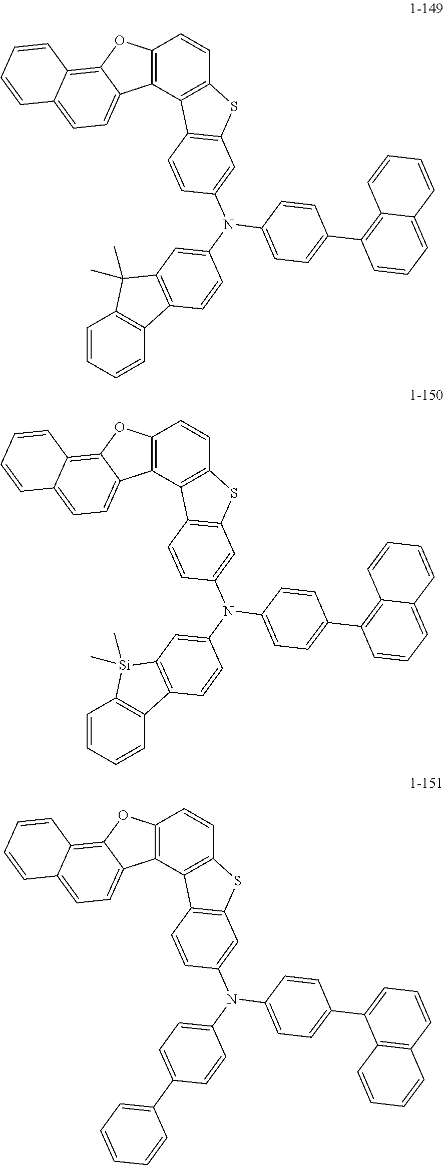 [SMILES: CC1(C)c2ccccc2-c2ccc(N(c3ccc(-c4cccc5ccccc45)cc3)c3ccc4c(c3)sc3ccc5oc6c7ccccc7ccc6c5c34)cc21 ; C[Si]1(C)c2ccccc2-c2ccc(N(c3ccc(-c4cccc5ccccc45)cc3)c3ccc4c(c3)sc3ccc5oc6c7ccccc7ccc6c5c34)cc21 ; c1ccc(-c2ccc(N(c3ccc(-c4cccc5ccccc45)cc3)c3ccc4c(c3)sc3ccc5oc6c7ccccc7ccc6c5c34)cc2)cc1]

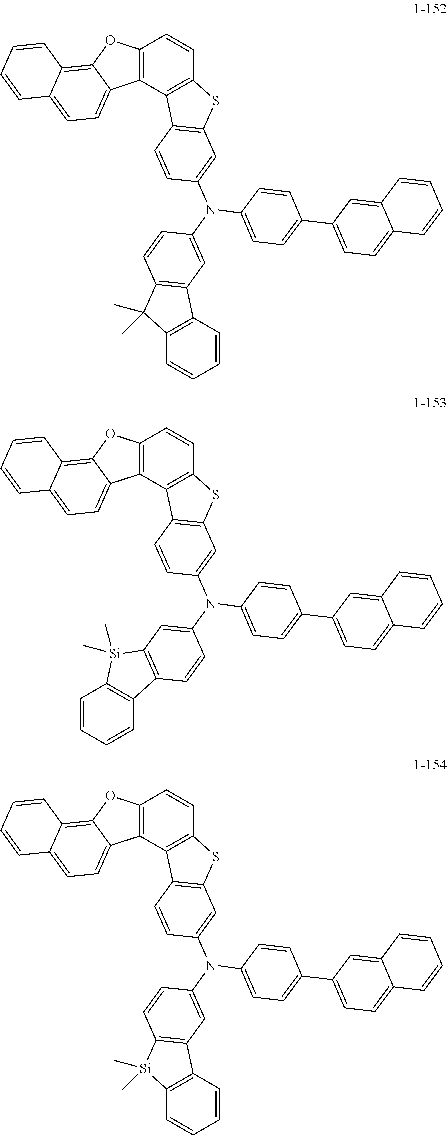 [SMILES: CC1(C)c2ccccc2-c2cc(N(c3ccc(-c4ccc5ccccc5c4)cc3)c3ccc4c(c3)sc3ccc5oc6c7ccccc7ccc6c5c34)ccc21 ; C[Si]1(C)c2ccccc2-c2ccc(N(c3ccc(-c4ccc5ccccc5c4)cc3)c3ccc4c(c3)sc3ccc5oc6c7ccccc7ccc6c5c34)cc21 ; C[Si]1(C)c2ccccc2-c2cc(N(c3ccc(-c4ccc5ccccc5c4)cc3)c3ccc4c(c3)sc3ccc5oc6c7ccccc7ccc6c5c34)ccc21]

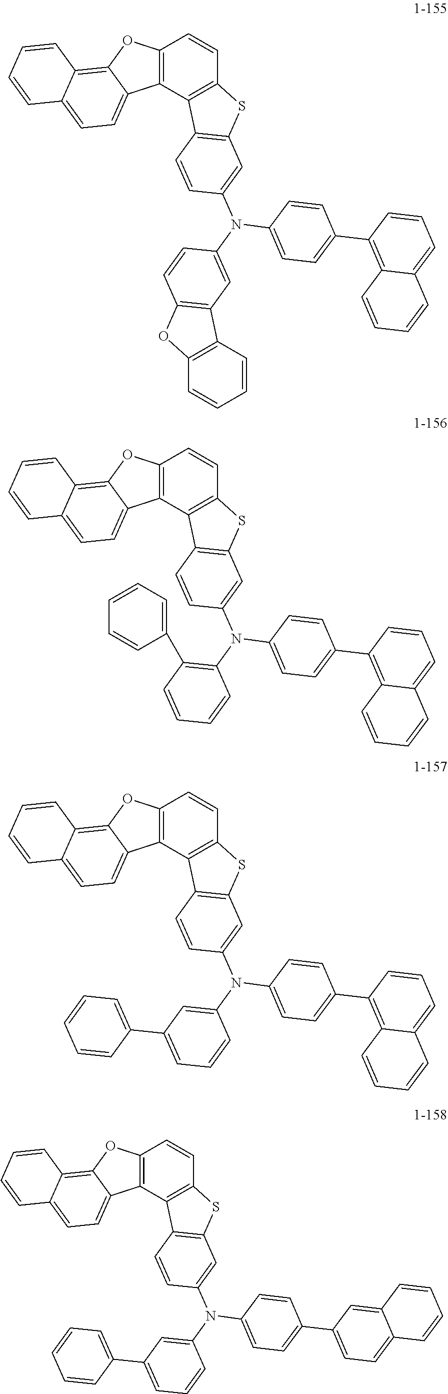 [SMILES: c1ccc2c(-c3ccc(N(c4ccc5c(c4)sc4ccc6oc7c8ccccc8ccc7c6c45)c4ccc5oc6ccccc6c5c4)cc3)cccc2c1 ; c1ccc(-c2ccccc2N(c2ccc(-c3cccc4ccccc34)cc2)c2ccc3c(c2)sc2ccc4oc5c6ccccc6ccc5c4c23)cc1 ; c1ccc(-c2cccc(N(c3ccc(-c4cccc5ccccc45)cc3)c3ccc4c(c3)sc3ccc5oc6c7ccccc7ccc6c5c34)c2)cc1 ; c1ccc(-c2cccc(N(c3ccc(-c4ccc5ccccc5c4)cc3)c3ccc4c(c3)sc3ccc5oc6c7ccccc7ccc6c5c34)c2)cc1]

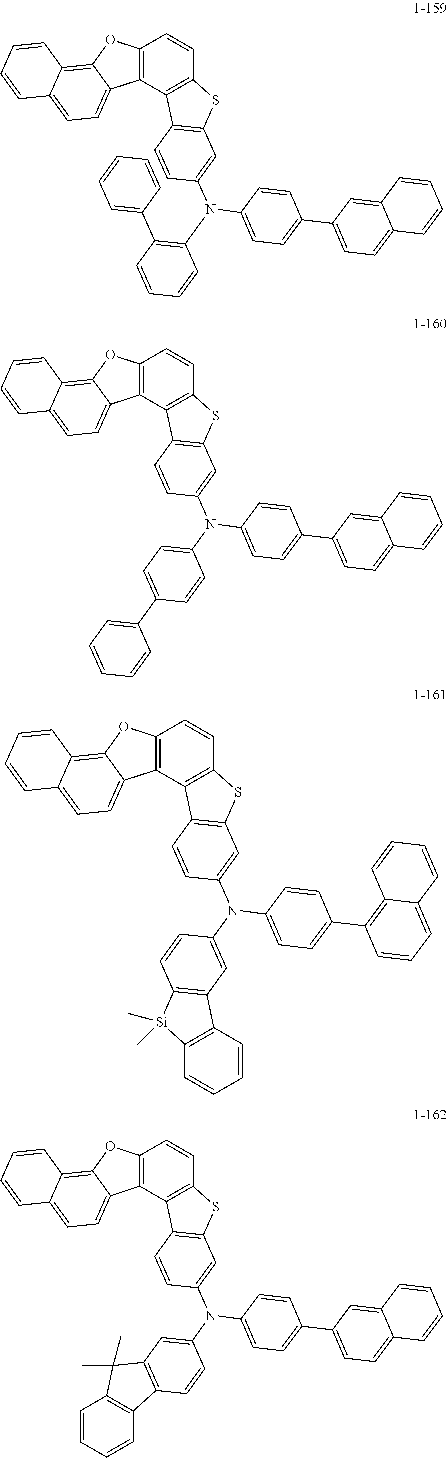 [SMILES: c1ccc(-c2ccccc2N(c2ccc(-c3ccc4ccccc4c3)cc2)c2ccc3c(c2)sc2ccc4oc5c6ccccc6ccc5c4c23)cc1 ; c1ccc(-c2ccc(N(c3ccc(-c4ccc5ccccc5c4)cc3)c3ccc4c(c3)sc3ccc5oc6c7ccccc7ccc6c5c34)cc2)cc1 ; C[Si]1(C)c2ccccc2-c2cc(N(c3ccc(-c4cccc5ccccc45)cc3)c3ccc4c(c3)sc3ccc5oc6c7ccccc7ccc6c5c34)ccc21 ; CC1(C)c2ccccc2-c2ccc(N(c3ccc(-c4ccc5ccccc5c4)cc3)c3ccc4c(c3)sc3ccc5oc6c7ccccc7ccc6c5c34)cc21]

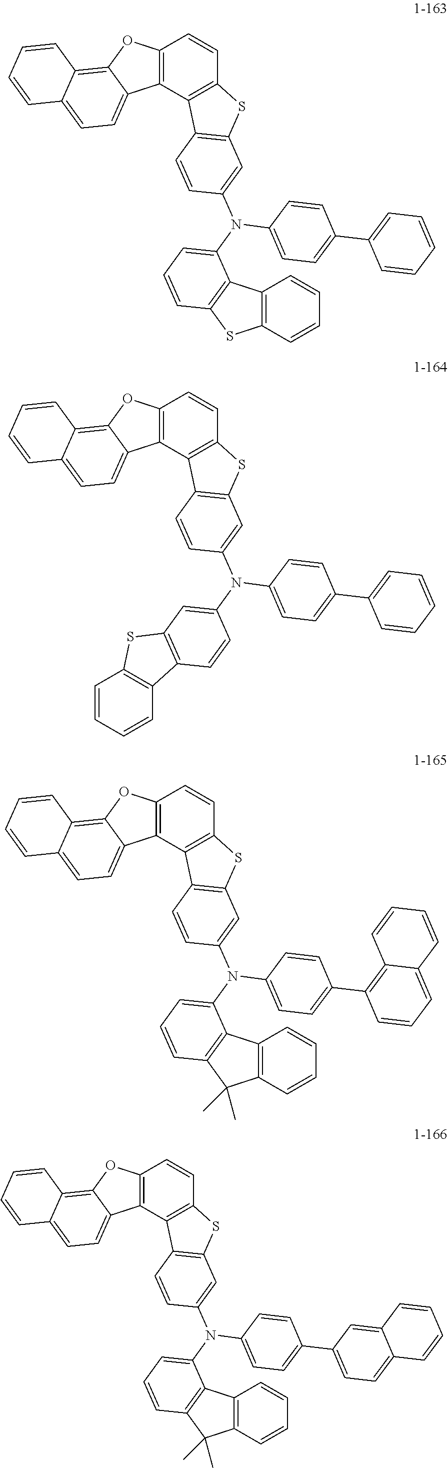 [SMILES: c1ccc(-c2ccc(N(c3ccc4c(c3)sc3ccc5oc6c7ccccc7ccc6c5c34)c3cccc4sc5ccccc5c34)cc2)cc1 ; c1ccc(-c2ccc(N(c3ccc4c(c3)sc3ccccc34)c3ccc4c(c3)sc3ccc5oc6c7ccccc7ccc6c5c34)cc2)cc1 ; CC1(C)c2ccccc2-c2c(N(c3ccc(-c4cccc5ccccc45)cc3)c3ccc4c(c3)sc3ccc5oc6c7ccccc7ccc6c5c34)cccc21 ; CC1(C)c2ccccc2-c2c(N(c3ccc(-c4ccc5ccccc5c4)cc3)c3ccc4c(c3)sc3ccc5oc6c7ccccc7ccc6c5c34)cccc21]

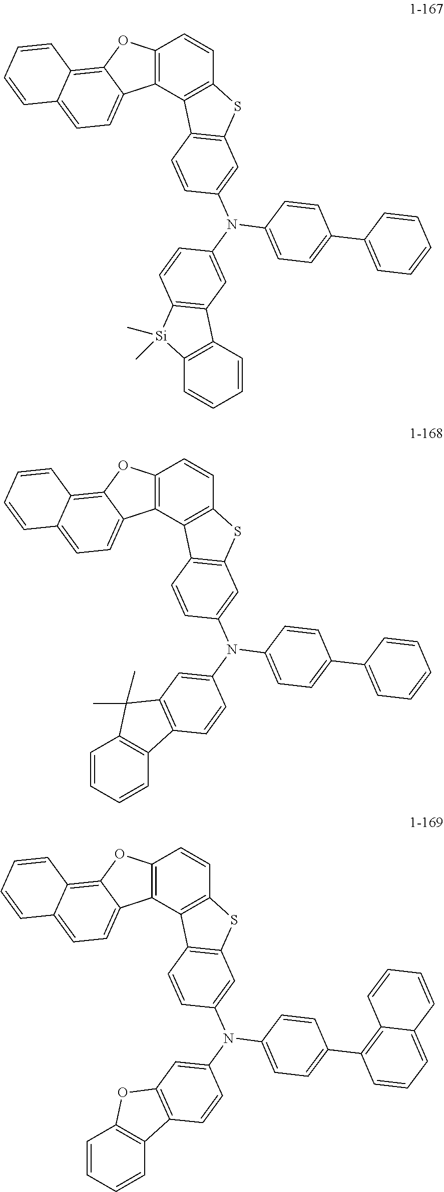 [SMILES: C[Si]1(C)c2ccccc2-c2cc(N(c3ccc(-c4ccccc4)cc3)c3ccc4c(c3)sc3ccc5oc6c7ccccc7ccc6c5c34)ccc21 ; CC1(C)c2ccccc2-c2ccc(N(c3ccc(-c4ccccc4)cc3)c3ccc4c(c3)sc3ccc5oc6c7ccccc7ccc6c5c34)cc21 ; c1ccc2c(-c3ccc(N(c4ccc5c(c4)oc4ccccc45)c4ccc5c(c4)sc4ccc6oc7c8ccccc8ccc7c6c45)cc3)cccc2c1]

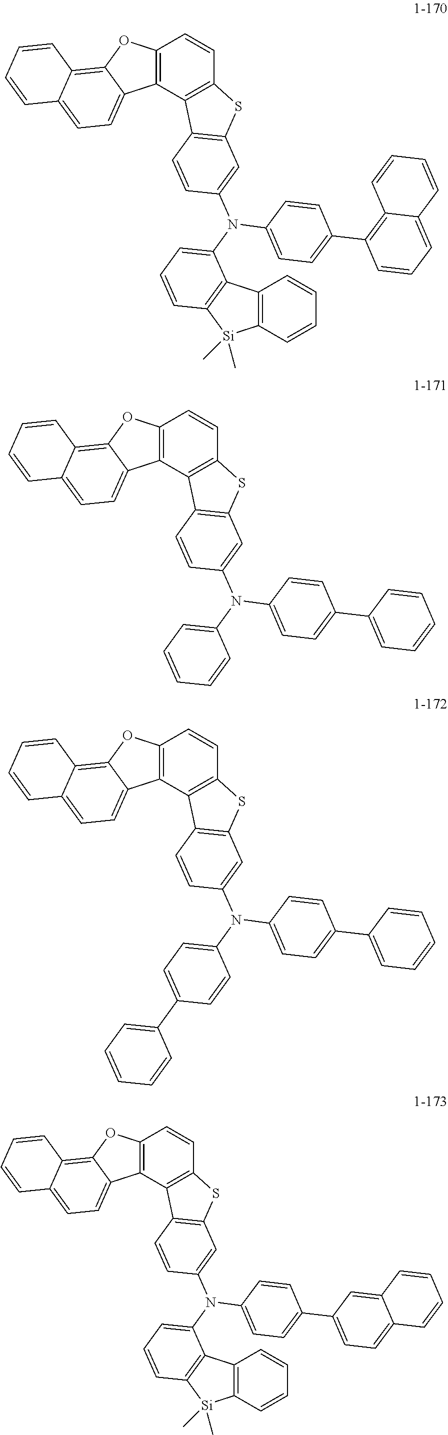

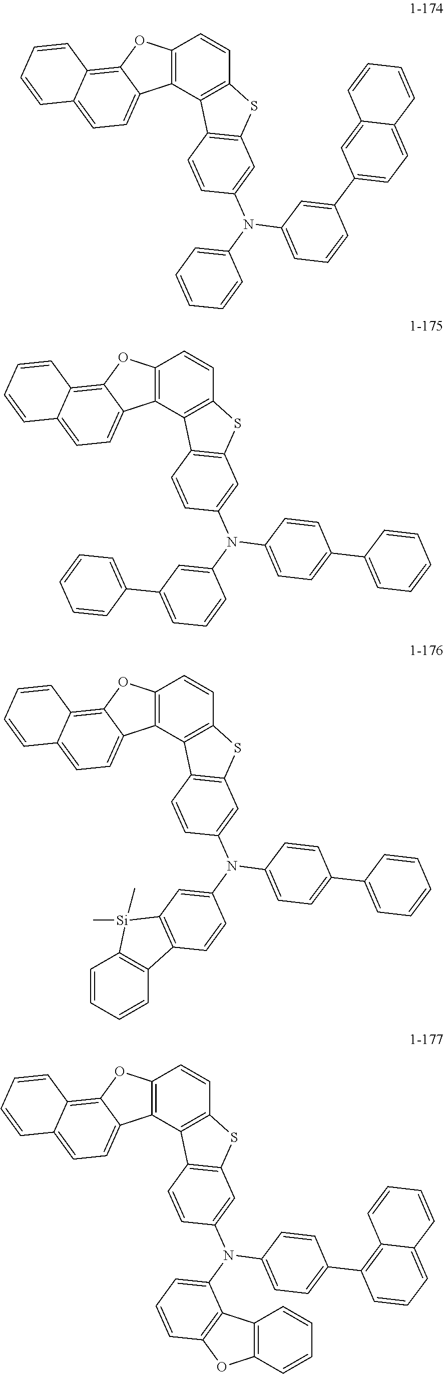

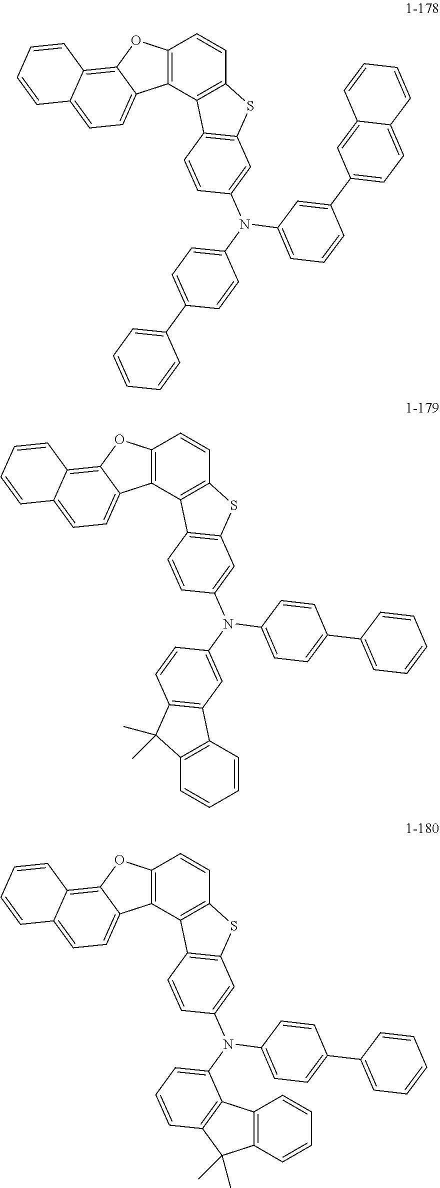 [SMILES: c1ccc(-c2ccc(N(c3cccc(-c4ccc5ccccc5c4)c3)c3ccc4c(c3)sc3ccc5oc6c7ccccc7ccc6c5c34)cc2)cc1 ; CC1(C)c2ccccc2-c2cc(N(c3ccc(-c4ccccc4)cc3)c3ccc4c(c3)sc3ccc5oc6c7ccccc7ccc6c5c34)ccc21 ; CC1(C)c2ccccc2-c2c(N(c3ccc(-c4ccccc4)cc3)c3ccc4c(c3)sc3ccc5oc6c7ccccc7ccc6c5c34)cccc21]

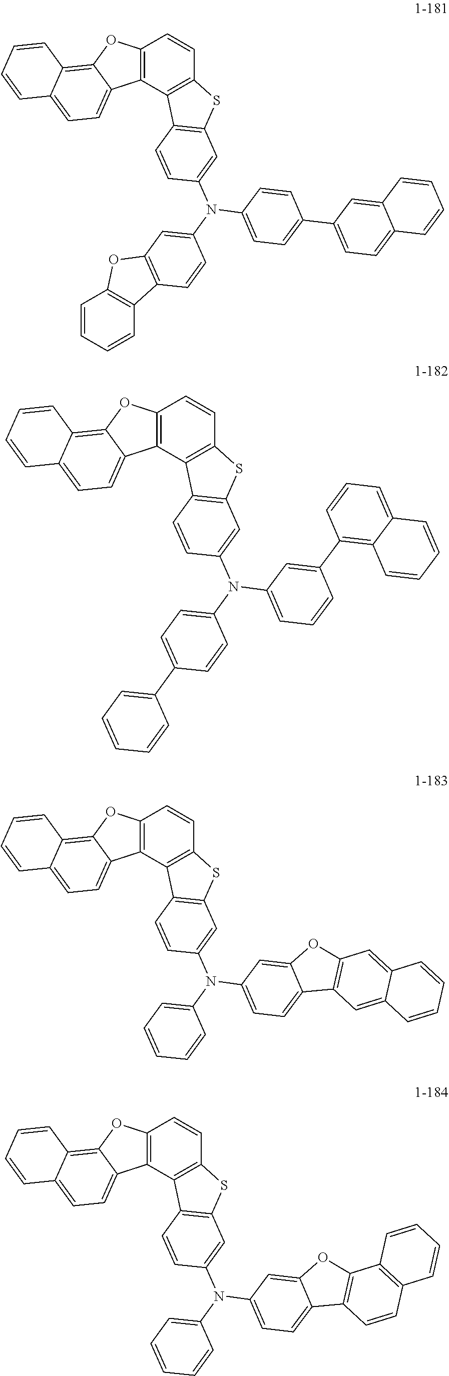 [SMILES: c1ccc2cc(-c3ccc(N(c4ccc5c(c4)oc4ccccc45)c4ccc5c(c4)sc4ccc6oc7c8ccccc8ccc7c6c45)cc3)ccc2c1 ; c1ccc(-c2ccc(N(c3cccc(-c4cccc5ccccc45)c3)c3ccc4c(c3)sc3ccc5oc6c7ccccc7ccc6c5c34)cc2)cc1 ; c1ccc(N(c2ccc3c(c2)oc2cc4ccccc4cc23)c2ccc3c(c2)sc2ccc4oc5c6ccccc6ccc5c4c23)cc1 ; c1ccc(N(c2ccc3c(c2)oc2c4ccccc4ccc32)c2ccc3c(c2)sc2ccc4oc5c6ccccc6ccc5c4c23)cc1]

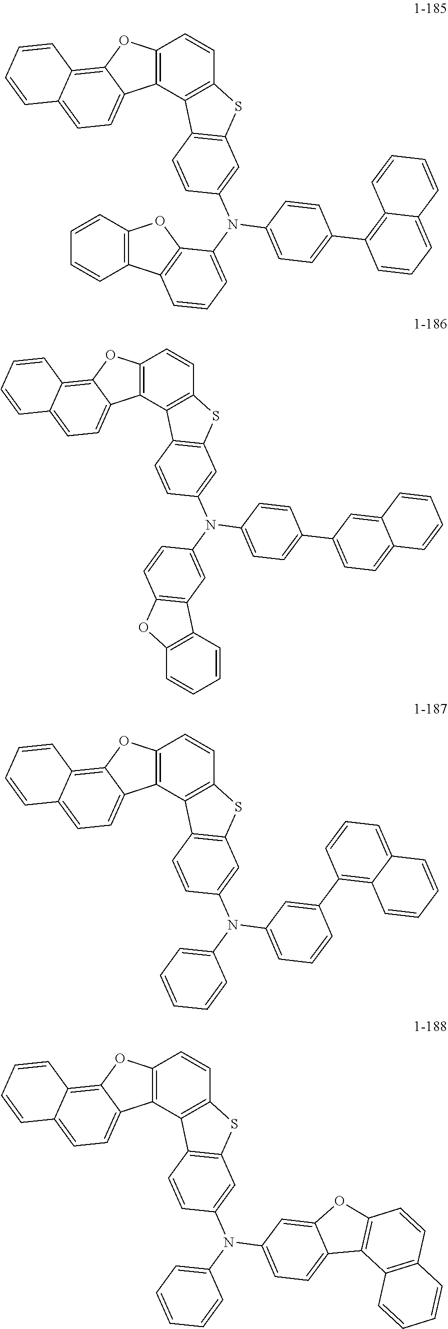

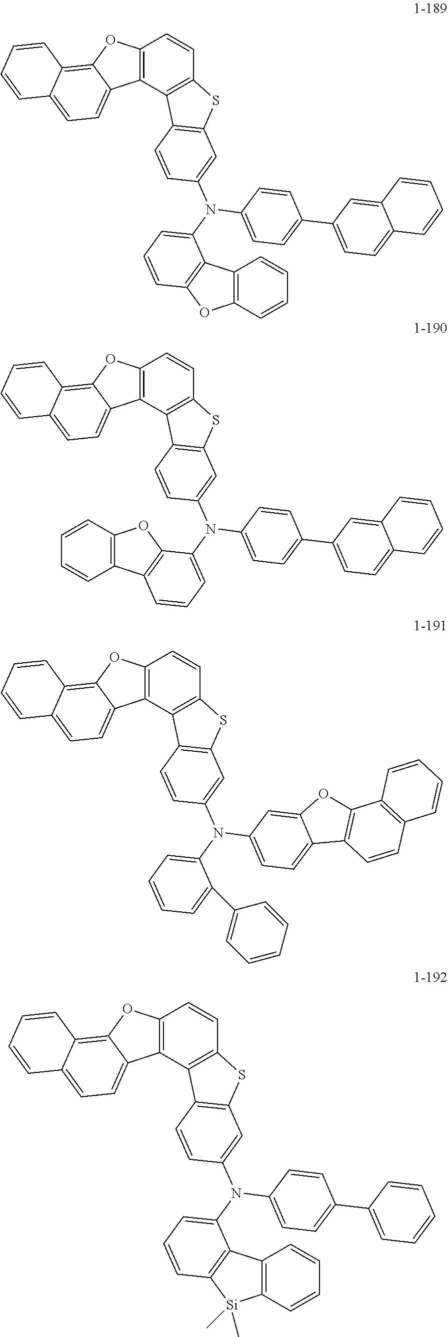 [SMILES: c1ccc2cc(-c3ccc(N(c4ccc5c(c4)sc4ccc6oc7c8ccccc8ccc7c6c45)c4cccc5oc6ccccc6c45)cc3)ccc2c1 ; c1ccc2cc(-c3ccc(N(c4ccc5c(c4)sc4ccc6oc7c8ccccc8ccc7c6c45)c4cccc5c4oc4ccccc45)cc3)ccc2c1 ; c1ccc(-c2ccccc2N(c2ccc3c(c2)oc2c4ccccc4ccc32)c2ccc3c(c2)sc2ccc4oc5c6ccccc6ccc5c4c23)cc1 ; C[Si]1(C)c2ccccc2-c2c(N(c3ccc(-c4ccccc4)cc3)c3ccc4c(c3)sc3ccc5oc6c7ccccc7ccc6c5c34)cccc21]

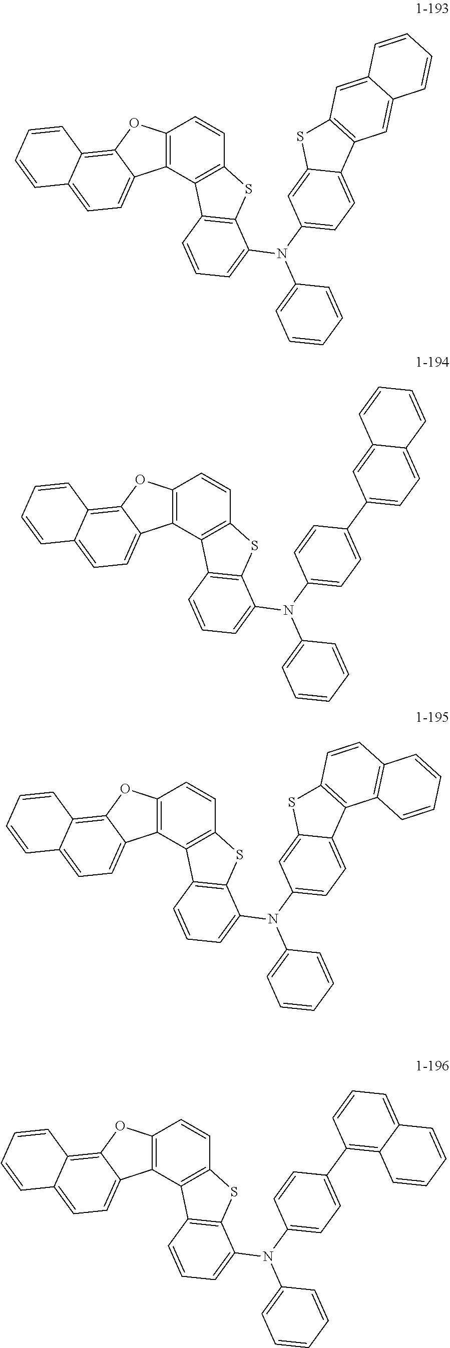 [SMILES: c1ccc(N(c2ccc3c(c2)sc2cc4ccccc4cc23)c2cccc3c2sc2ccc4oc5c6ccccc6ccc5c4c23)cc1 ; c1ccc(N(c2ccc(-c3ccc4ccccc4c3)cc2)c2cccc3c2sc2ccc4oc5c6ccccc6ccc5c4c23)cc1 ; c1ccc(N(c2ccc3c(c2)sc2ccc4ccccc4c23)c2cccc3c2sc2ccc4oc5c6ccccc6ccc5c4c23)cc1 ; c1ccc(N(c2ccc(-c3cccc4ccccc34)cc2)c2cccc3c2sc2ccc4oc5c6ccccc6ccc5c4c23)cc1]

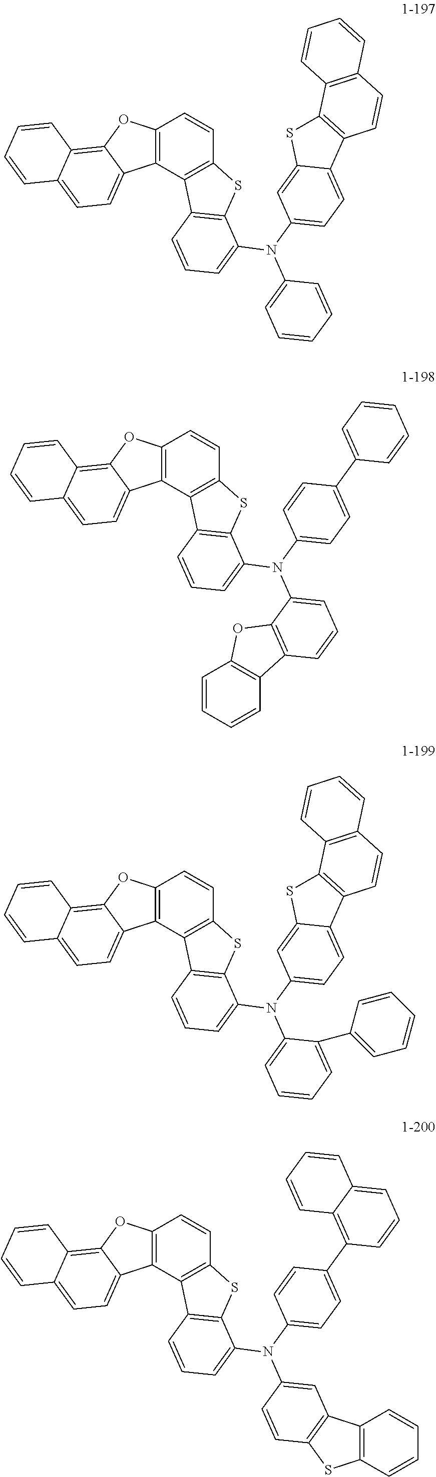 [SMILES: c1ccc(N(c2ccc3c(c2)sc2c4ccccc4ccc32)c2cccc3c2sc2ccc4oc5c6ccccc6ccc5c4c23)cc1 ; c1ccc(-c2ccc(N(c3cccc4c3oc3ccccc34)c3cccc4c3sc3ccc5oc6c7ccccc7ccc6c5c34)cc2)cc1 ; c1ccc(-c2ccccc2N(c2ccc3c(c2)sc2c4ccccc4ccc32)c2cccc3c2sc2ccc4oc5c6ccccc6ccc5c4c23)cc1 ; c1ccc2c(-c3ccc(N(c4ccc5sc6ccccc6c5c4)c4cccc5c4sc4ccc6oc7c8ccccc8ccc7c6c45)cc3)cccc2c1]

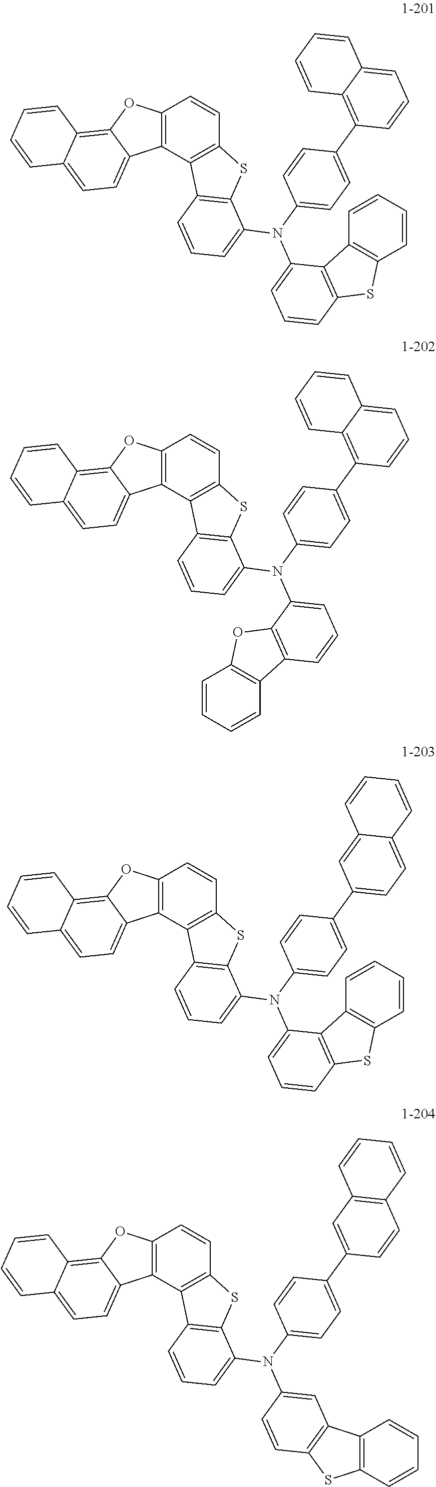 [SMILES: c1ccc2c(-c3ccc(N(c4cccc5c4sc4ccc6oc7c8ccccc8ccc7c6c45)c4cccc5sc6ccccc6c45)cc3)cccc2c1 ; c1ccc2c(-c3ccc(N(c4cccc5c4oc4ccccc45)c4cccc5c4sc4ccc6oc7c8ccccc8ccc7c6c45)cc3)cccc2c1 ; c1ccc2cc(-c3ccc(N(c4cccc5c4sc4ccc6oc7c8ccccc8ccc7c6c45)c4cccc5sc6ccccc6c45)cc3)ccc2c1 ; c1ccc2cc(-c3ccc(N(c4ccc5sc6ccccc6c5c4)c4cccc5c4sc4ccc6oc7c8ccccc8ccc7c6c45)cc3)ccc2c1]

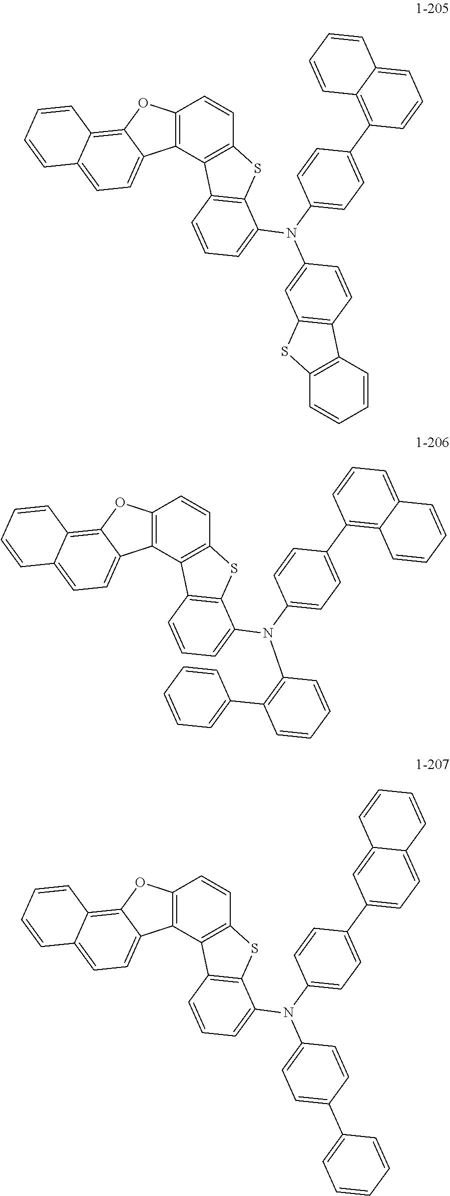

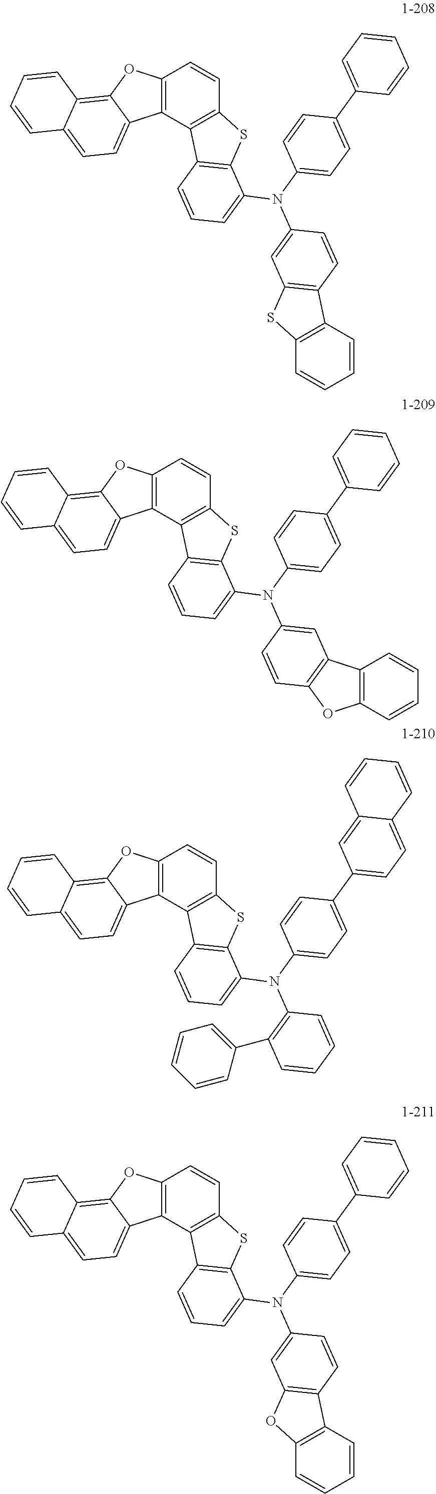

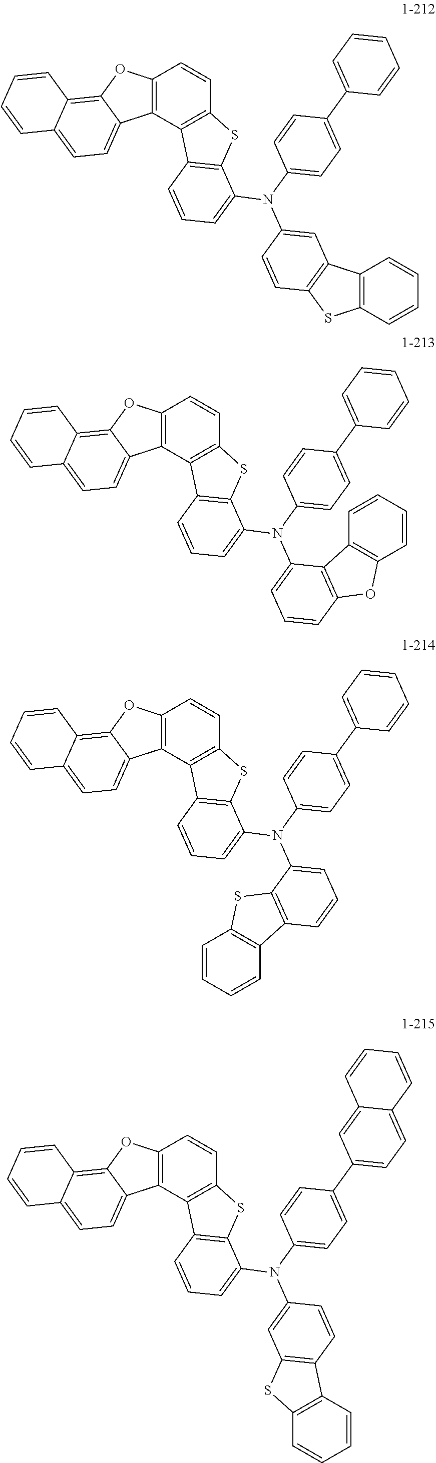 [SMILES: c1ccc(-c2ccc(N(c3ccc4sc5ccccc5c4c3)c3cccc4c3sc3ccc5oc6c7ccccc7ccc6c5c34)cc2)cc1 ; c1ccc(-c2ccc(N(c3cccc4c3sc3ccc5oc6c7ccccc7ccc6c5c34)c3cccc4oc5ccccc5c34)cc2)cc1 ; c1ccc(-c2ccc(N(c3cccc4c3sc3ccccc34)c3cccc4c3sc3ccc5oc6c7ccccc7ccc6c5c34)cc2)cc1 ; c1ccc2cc(-c3ccc(N(c4ccc5c(c4)sc4ccccc45)c4cccc5c4sc4ccc6oc7c8ccccc8ccc7c6c45)cc3)ccc2c1]

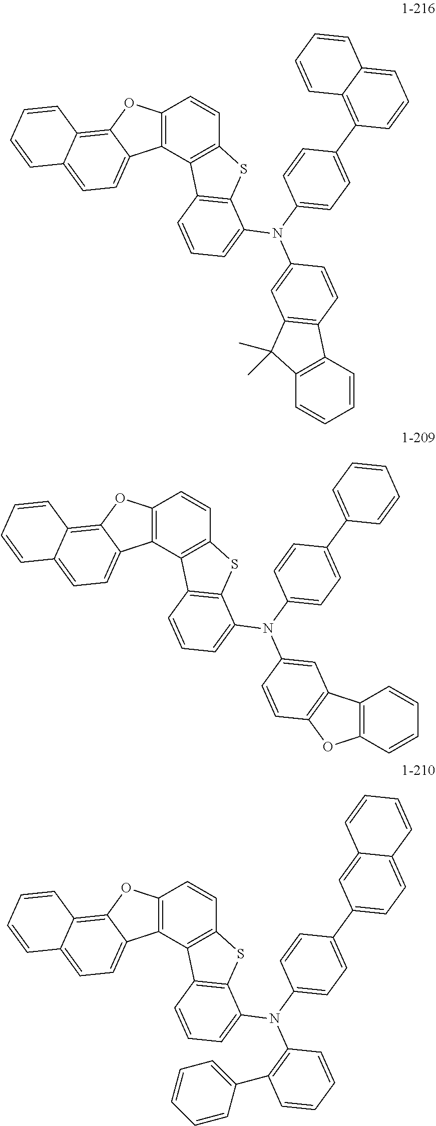 [SMILES: CC1(C)c2ccccc2-c2ccc(N(c3ccc(-c4cccc5ccccc45)cc3)c3cccc4c3sc3ccc5oc6c7ccccc7ccc6c5c34)cc21 ; c1ccc(-c2ccc(N(c3ccc4oc5ccccc5c4c3)c3cccc4c3sc3ccc5oc6c7ccccc7ccc6c5c34)cc2)cc1 ; c1ccc(-c2ccccc2N(c2ccc(-c3ccc4ccccc4c3)cc2)c2cccc3c2sc2ccc4oc5c6ccccc6ccc5c4c23)cc1]

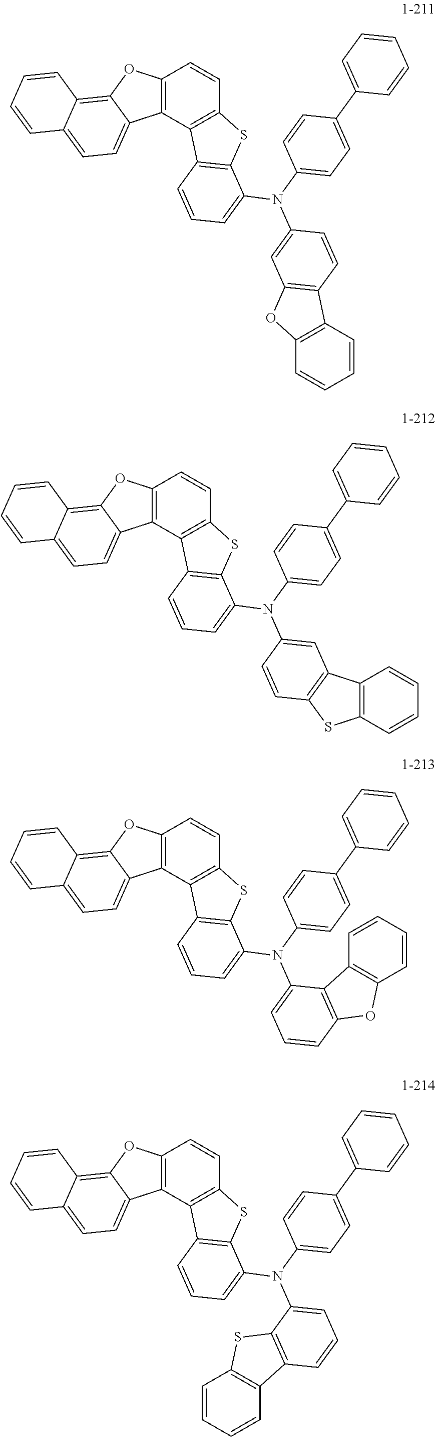 [SMILES: c1ccc(-c2ccc(N(c3ccc4c(c3)oc3ccccc34)c3cccc4c3sc3ccc5oc6c7ccccc7ccc6c5c34)cc2)cc1 ; c1ccc(-c2ccc(N(c3ccc4sc5ccccc5c4c3)c3cccc4c3sc3ccc5oc6c7ccccc7ccc6c5c34)cc2)cc1 ; c1ccc(-c2ccc(N(c3cccc4c3sc3ccc5oc6c7ccccc7ccc6c5c34)c3cccc4oc5ccccc5c34)cc2)cc1 ; c1ccc(-c2ccc(N(c3cccc4c3sc3ccccc34)c3cccc4c3sc3ccc5oc6c7ccccc7ccc6c5c34)cc2)cc1]

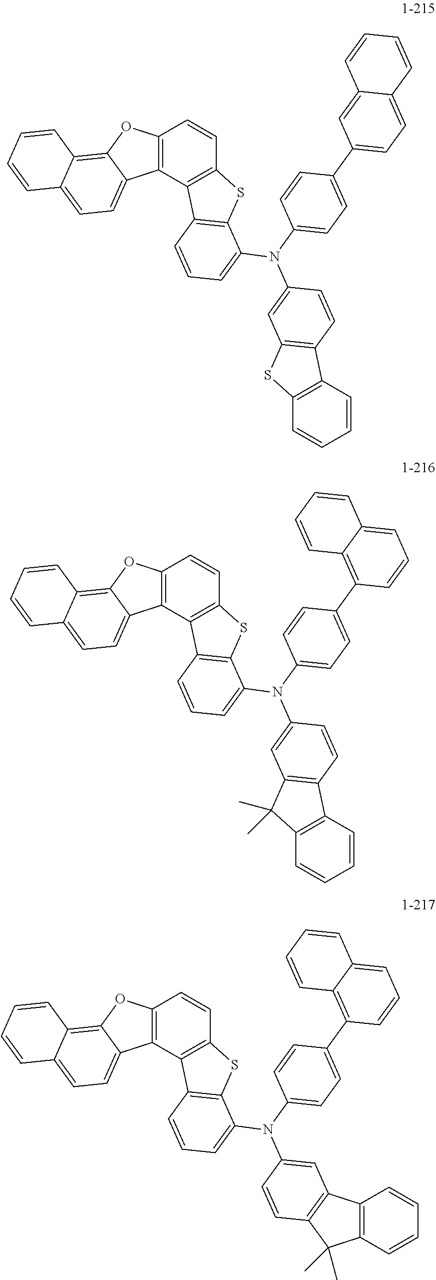

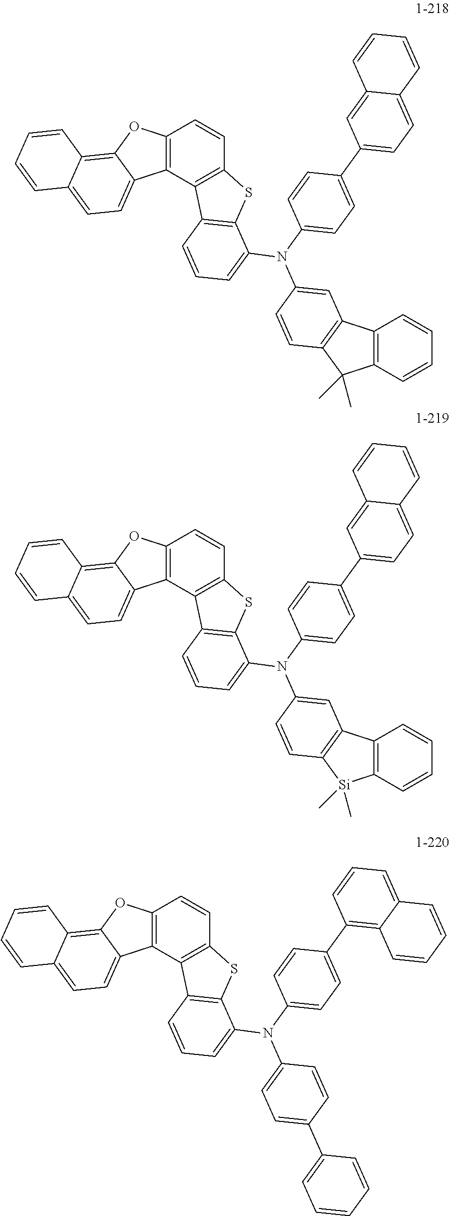

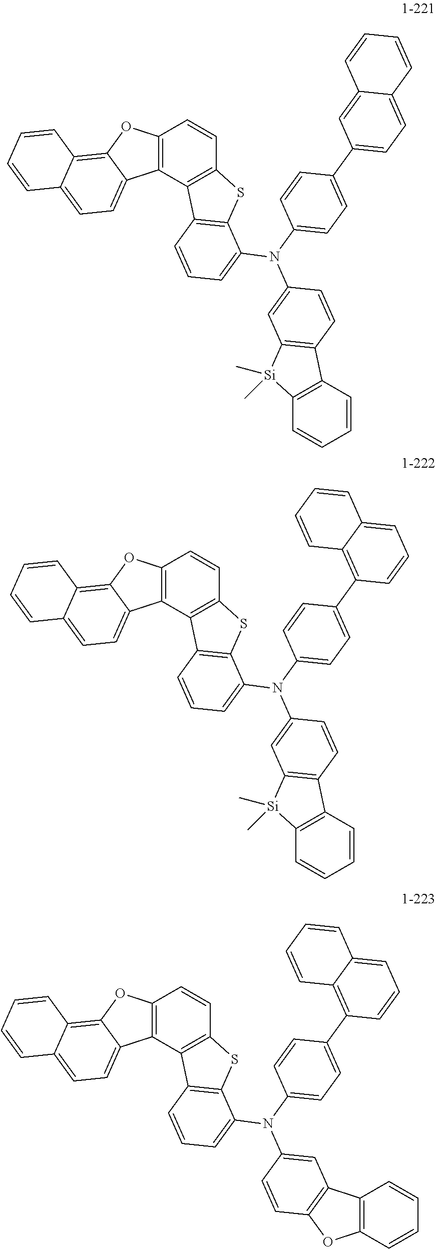

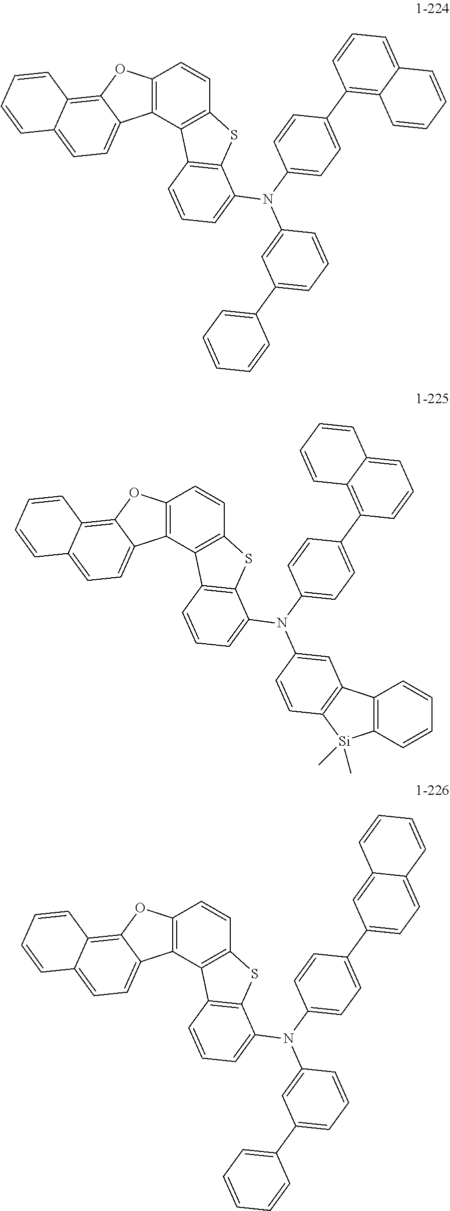 [SMILES: c1ccc(-c2cccc(N(c3ccc(-c4cccc5ccccc45)cc3)c3cccc4c3sc3ccc5oc6c7ccccc7ccc6c5c34)c2)cc1 ; C[Si]1(C)c2ccccc2-c2cc(N(c3ccc(-c4cccc5ccccc45)cc3)c3cccc4c3sc3ccc5oc6c7ccccc7ccc6c5c34)ccc21 ; c1ccc(-c2cccc(N(c3ccc(-c4ccc5ccccc5c4)cc3)c3cccc4c3sc3ccc5oc6c7ccccc7ccc6c5c34)c2)cc1]

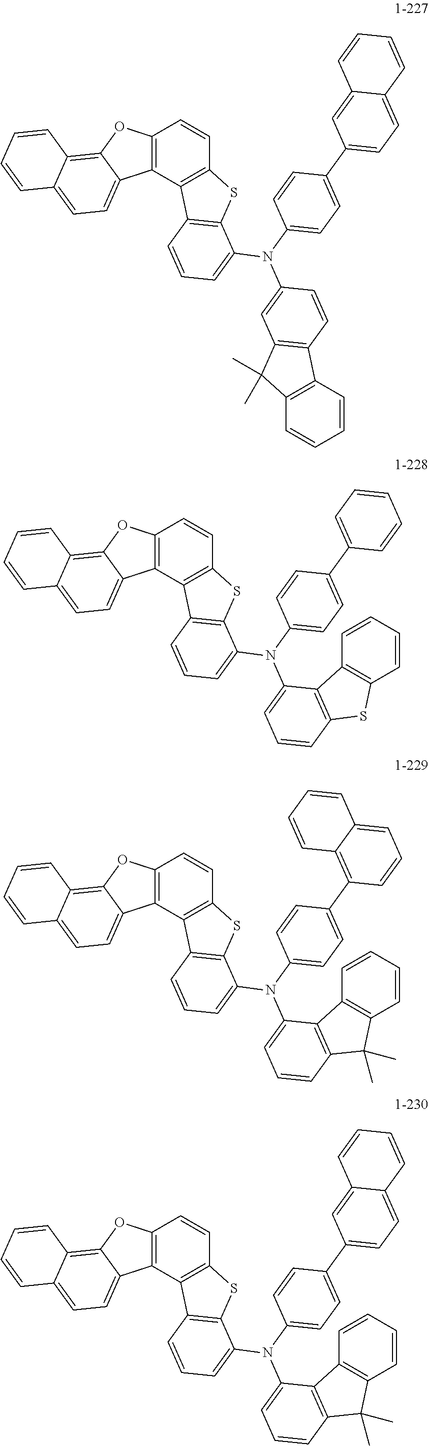

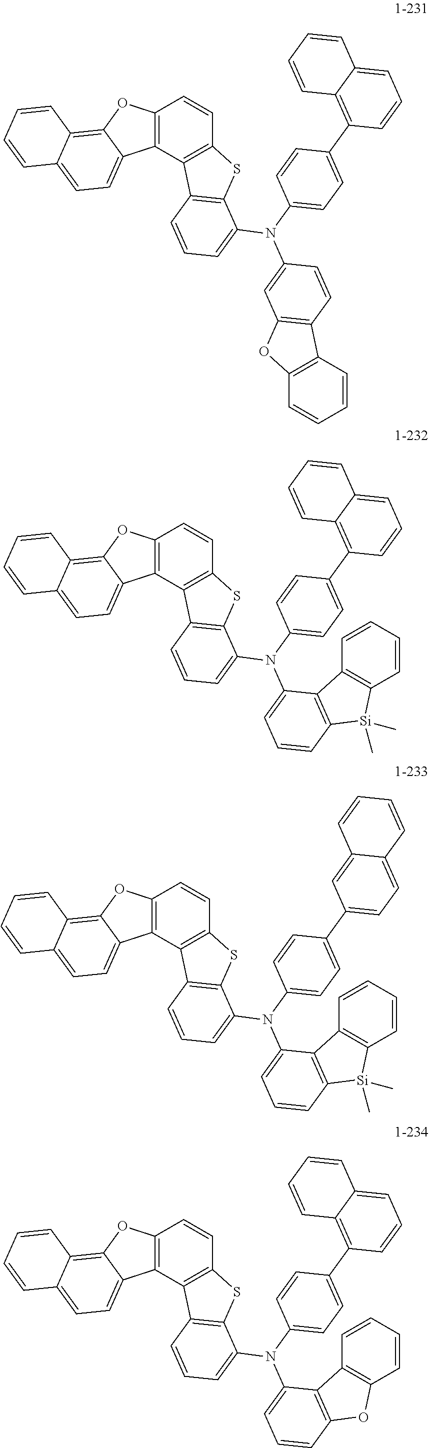

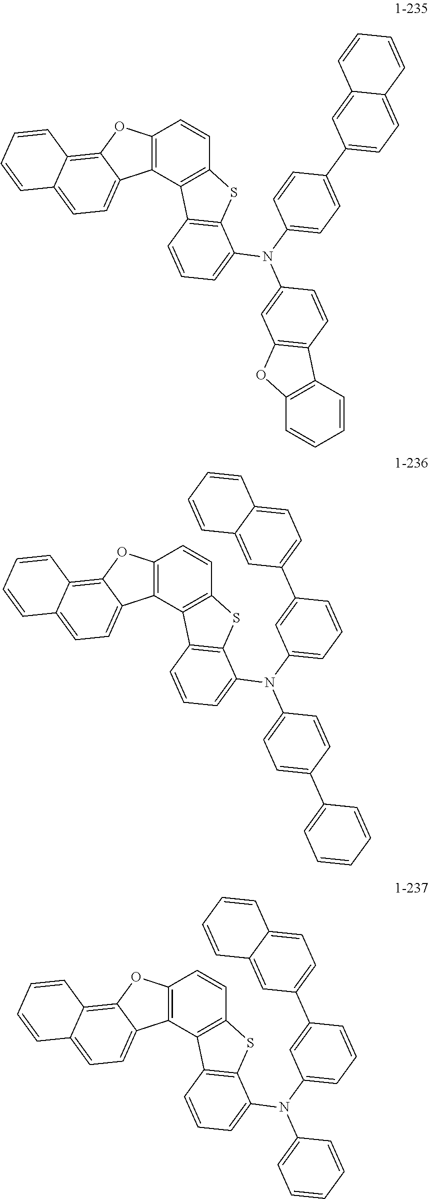 [SMILES: c1ccc2cc(-c3ccc(N(c4ccc5c(c4)oc4ccccc45)c4cccc5c4sc4ccc6oc7c8ccccc8ccc7c6c45)cc3)ccc2c1 ; c1ccc(-c2ccc(N(c3cccc(-c4ccc5ccccc5c4)c3)c3cccc4c3sc3ccc5oc6c7ccccc7ccc6c5c34)cc2)cc1 ; c1ccc(N(c2cccc(-c3ccc4ccccc4c3)c2)c2cccc3c2sc2ccc4oc5c6ccccc6ccc5c4c23)cc1]

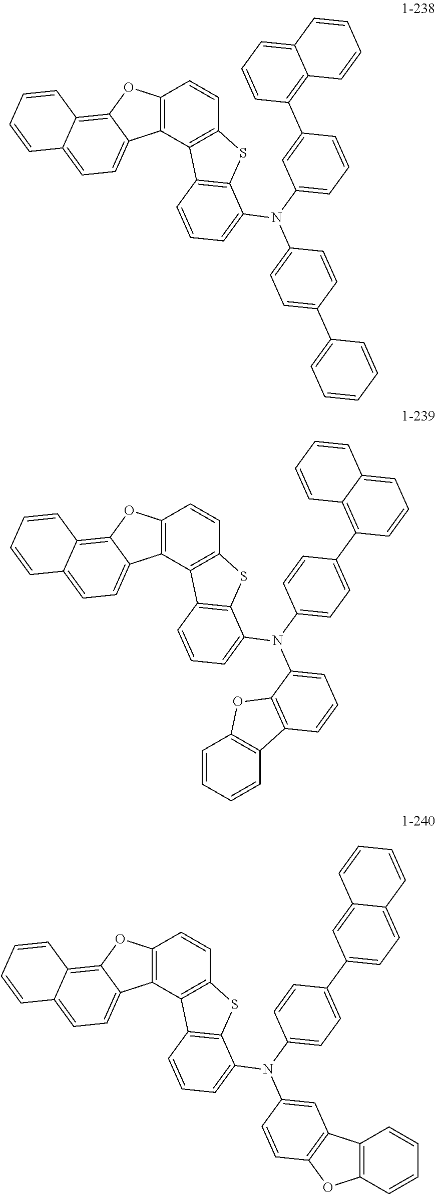

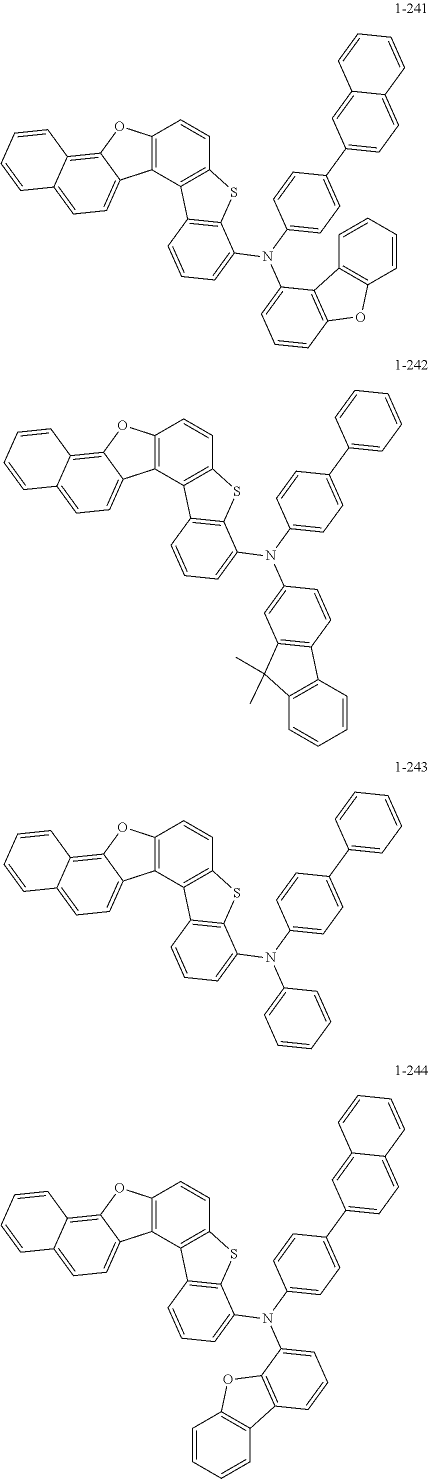 [SMILES: c1ccc2cc(-c3ccc(N(c4cccc5c4sc4ccc6oc7c8ccccc8ccc7c6c45)c4cccc5oc6ccccc6c45)cc3)ccc2c1 ; CC1(C)c2ccccc2-c2ccc(N(c3ccc(-c4ccccc4)cc3)c3cccc4c3sc3ccc5oc6c7ccccc7ccc6c5c34)cc21 ; c1ccc(-c2ccc(N(c3ccccc3)c3cccc4c3sc3ccc5oc6c7ccccc7ccc6c5c34)cc2)cc1 ; c1ccc2cc(-c3ccc(N(c4cccc5c4oc4ccccc45)c4cccc5c4sc4ccc6oc7c8ccccc8ccc7c6c45)cc3)ccc2c1]

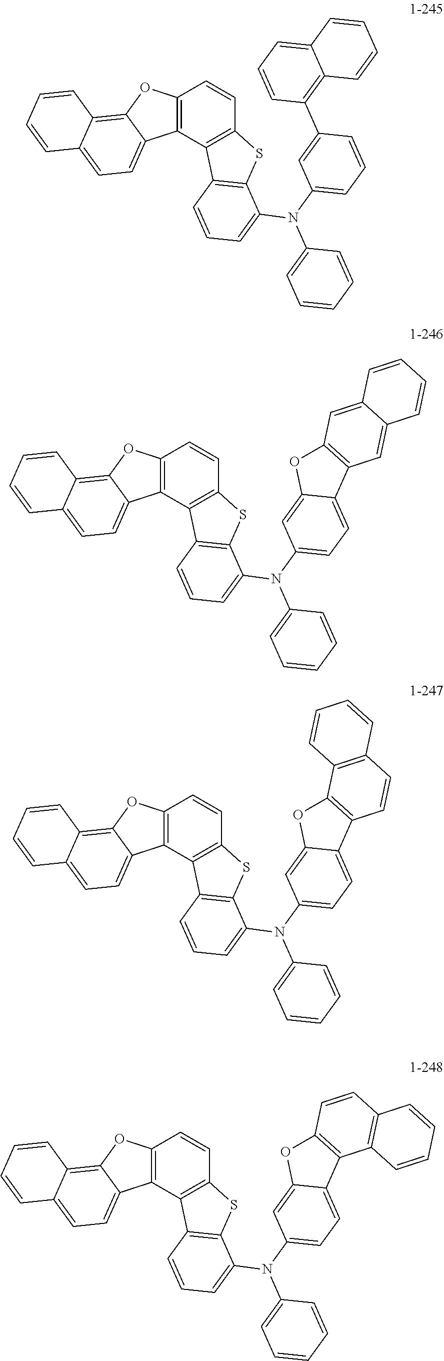

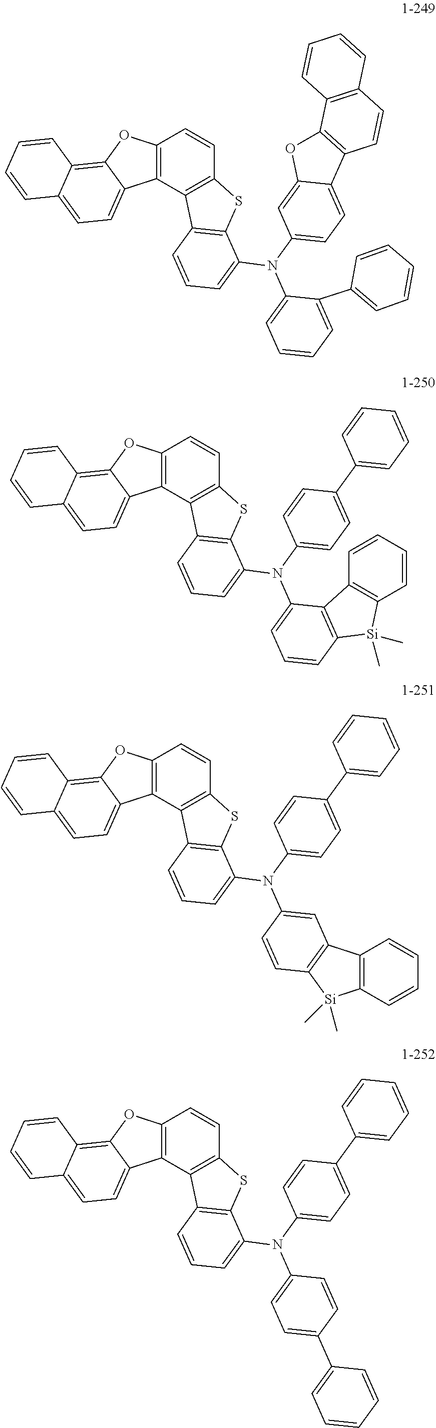 [SMILES: c1ccc(-c2ccccc2N(c2ccc3c(c2)oc2c4ccccc4ccc32)c2cccc3c2sc2ccc4oc5c6ccccc6ccc5c4c23)cc1 ; C[Si]1(C)c2ccccc2-c2c(N(c3ccc(-c4ccccc4)cc3)c3cccc4c3sc3ccc5oc6c7ccccc7ccc6c5c34)cccc21 ; C[Si]1(C)c2ccccc2-c2cc(N(c3ccc(-c4ccccc4)cc3)c3cccc4c3sc3ccc5oc6c7ccccc7ccc6c5c34)ccc21 ; c1ccc(-c2ccc(N(c3ccc(-c4ccccc4)cc3)c3cccc4c3sc3ccc5oc6c7ccccc7ccc6c5c34)cc2)cc1]

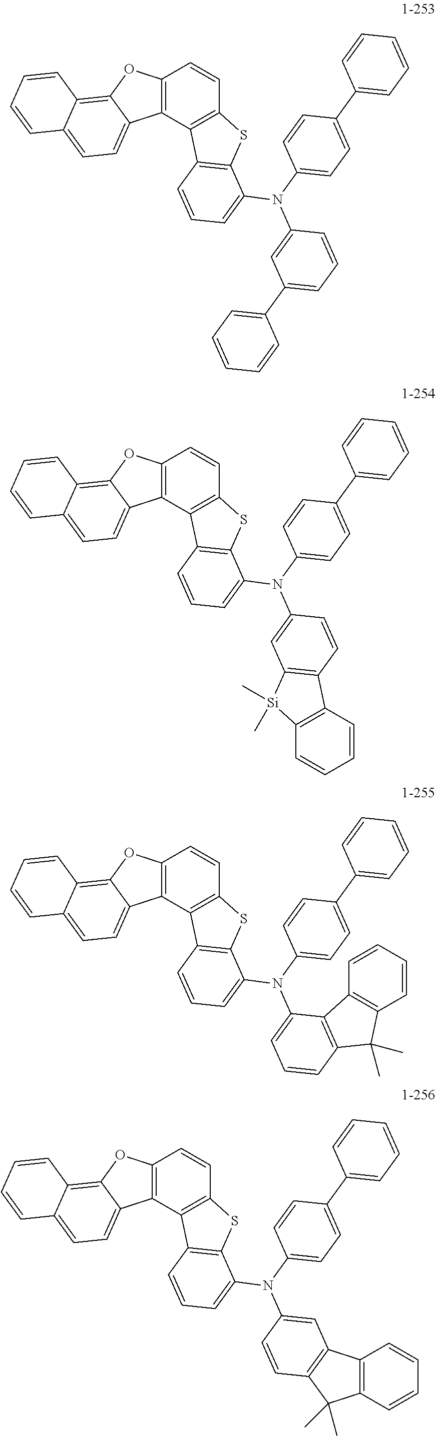 [SMILES: c1ccc(-c2ccc(N(c3cccc(-c4ccccc4)c3)c3cccc4c3sc3ccc5oc6c7ccccc7ccc6c5c34)cc2)cc1 ; C[Si]1(C)c2ccccc2-c2ccc(N(c3ccc(-c4ccccc4)cc3)c3cccc4c3sc3ccc5oc6c7ccccc7ccc6c5c34)cc21 ; CC1(C)c2ccccc2-c2c(N(c3ccc(-c4ccccc4)cc3)c3cccc4c3sc3ccc5oc6c7ccccc7ccc6c5c34)cccc21 ; CC1(C)c2ccccc2-c2cc(N(c3ccc(-c4ccccc4)cc3)c3cccc4c3sc3ccc5oc6c7ccccc7ccc6c5c34)ccc21]

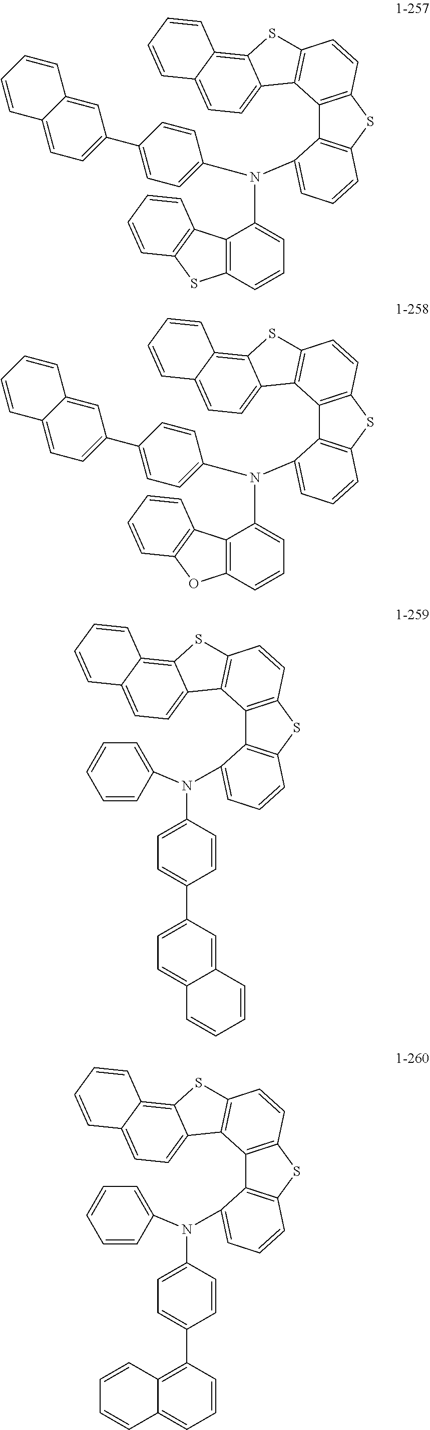

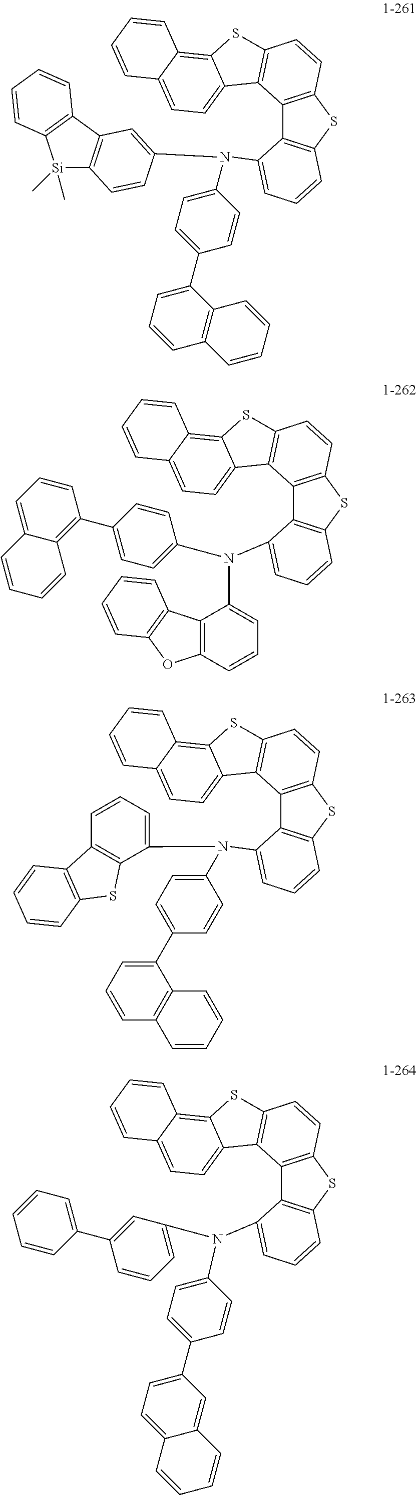

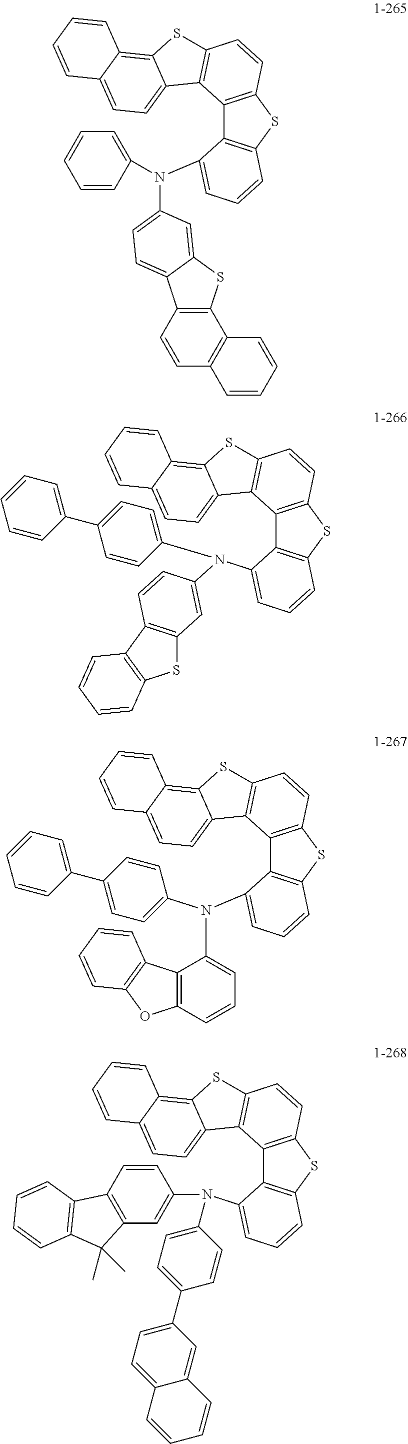

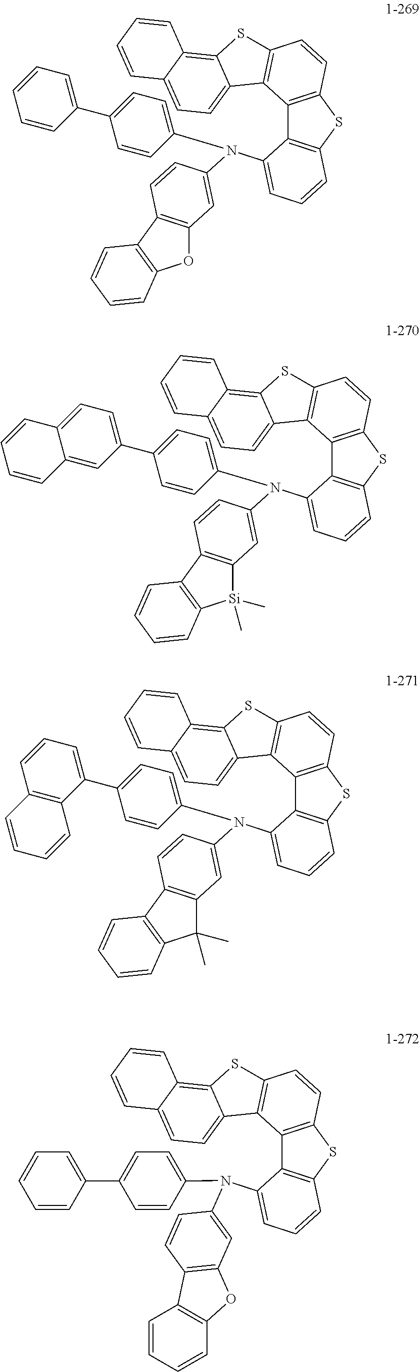 [SMILES: c1ccc(-c2ccc(N(c3ccc4c(c3)oc3ccccc34)c3cccc4sc5ccc6sc7c8ccccc8ccc7c6c5c34)cc2)cc1 ; C[Si]1(C)c2ccccc2-c2ccc(N(c3ccc(-c4ccc5ccccc5c4)cc3)c3cccc4sc5ccc6sc7c8ccccc8ccc7c6c5c34)cc21 ; CC1(C)c2ccccc2-c2ccc(N(c3ccc(-c4cccc5ccccc45)cc3)c3cccc4sc5ccc6sc7c8ccccc8ccc7c6c5c34)cc21 ; c1ccc(-c2ccc(N(c3ccc4c(c3)oc3ccccc34)c3cccc4sc5ccc6sc7c8ccccc8ccc7c6c5c34)cc2)cc1]

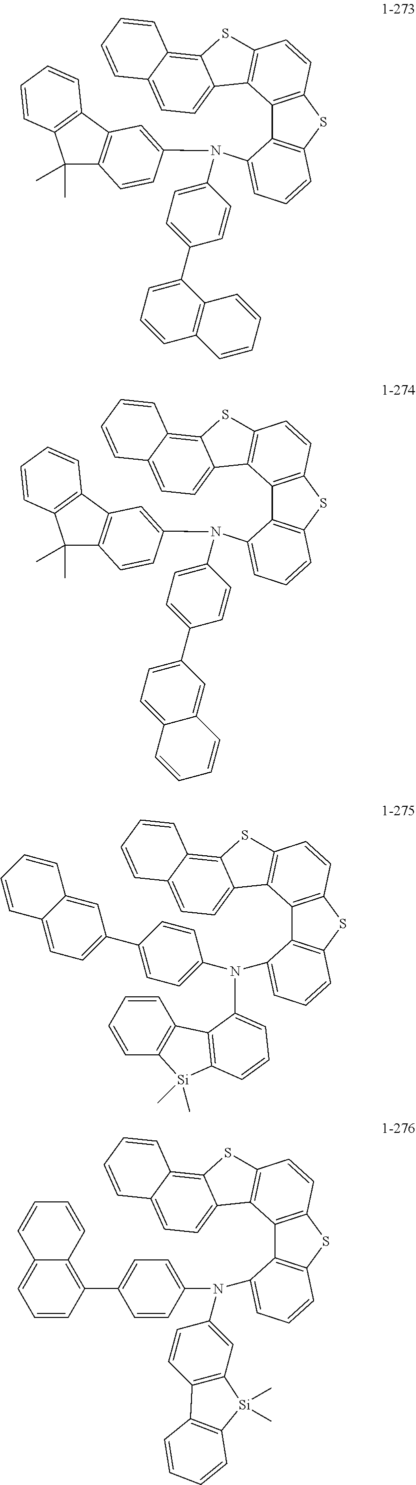

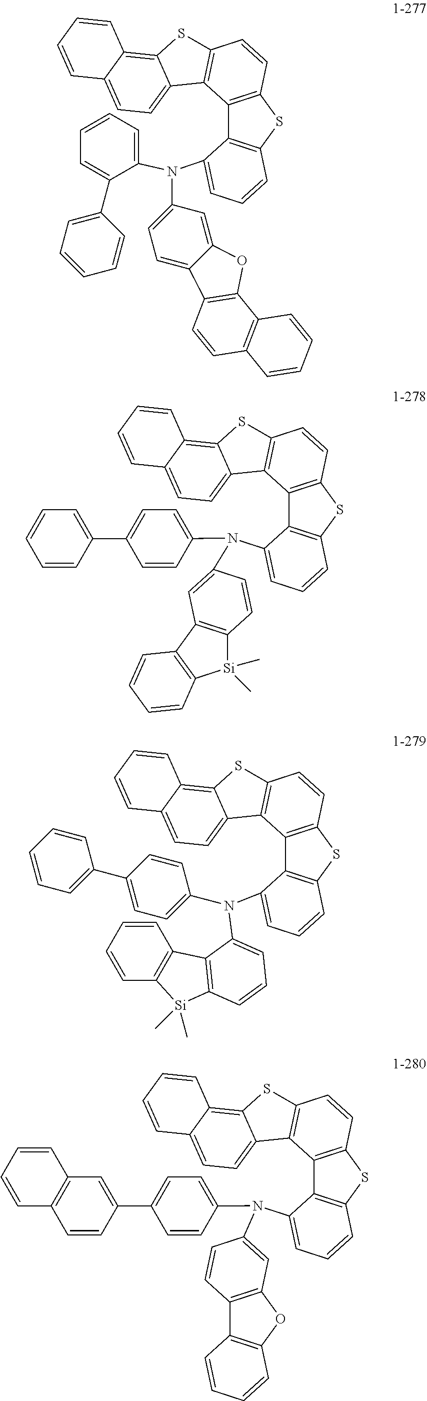

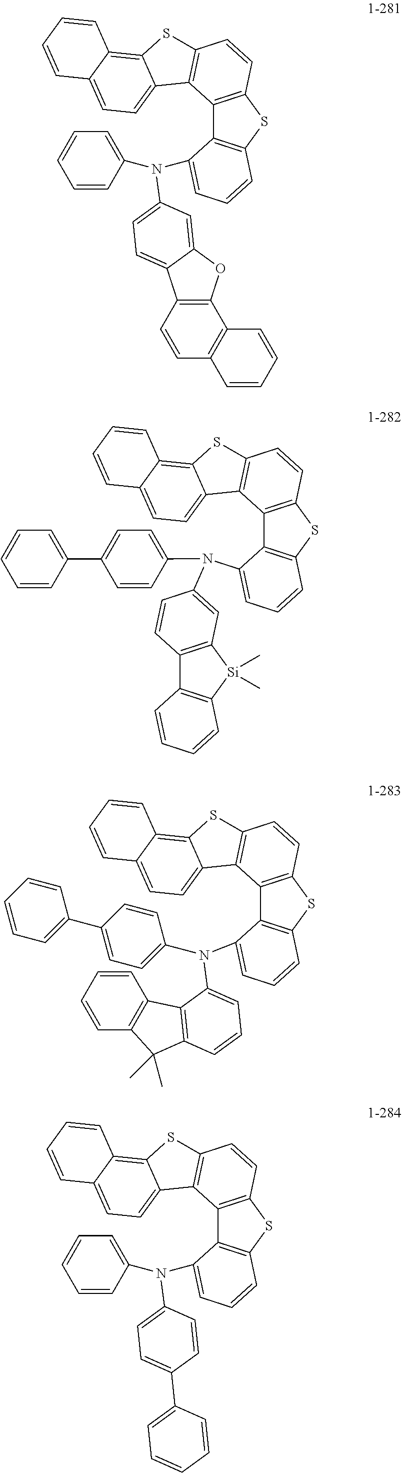 [SMILES: c1ccc(N(c2ccc3c(c2)oc2c4ccccc4ccc32)c2cccc3sc4ccc5sc6c7ccccc7ccc6c5c4c23)cc1 ; C[Si]1(C)c2ccccc2-c2ccc(N(c3ccc(-c4ccccc4)cc3)c3cccc4sc5ccc6sc7c8ccccc8ccc7c6c5c34)cc21 ; CC1(C)c2ccccc2-c2c(N(c3ccc(-c4ccccc4)cc3)c3cccc4sc5ccc6sc7c8ccccc8ccc7c6c5c34)cccc21 ; c1ccc(-c2ccc(N(c3ccccc3)c3cccc4sc5ccc6sc7c8ccccc8ccc7c6c5c34)cc2)cc1]

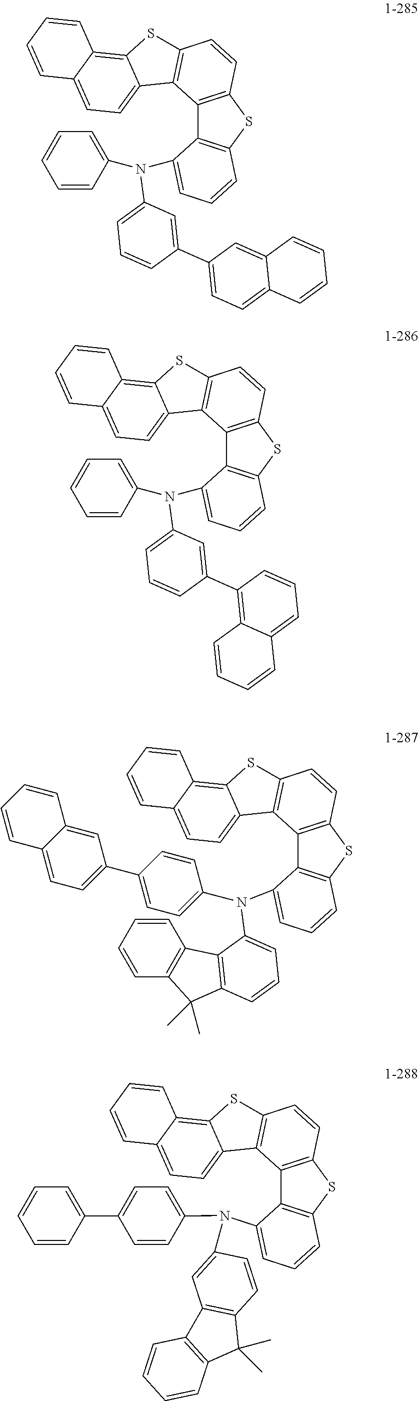 [SMILES: c1ccc(N(c2cccc(-c3ccc4ccccc4c3)c2)c2cccc3sc4ccc5sc6c7ccccc7ccc6c5c4c23)cc1 ; c1ccc(N(c2cccc(-c3cccc4ccccc34)c2)c2cccc3sc4ccc5sc6c7ccccc7ccc6c5c4c23)cc1 ; CC1(C)c2ccccc2-c2c(N(c3ccc(-c4ccc5ccccc5c4)cc3)c3cccc4sc5ccc6sc7c8ccccc8ccc7c6c5c34)cccc21 ; CC1(C)c2ccccc2-c2cc(N(c3ccc(-c4ccccc4)cc3)c3cccc4sc5ccc6sc7c8ccccc8ccc7c6c5c34)ccc21]

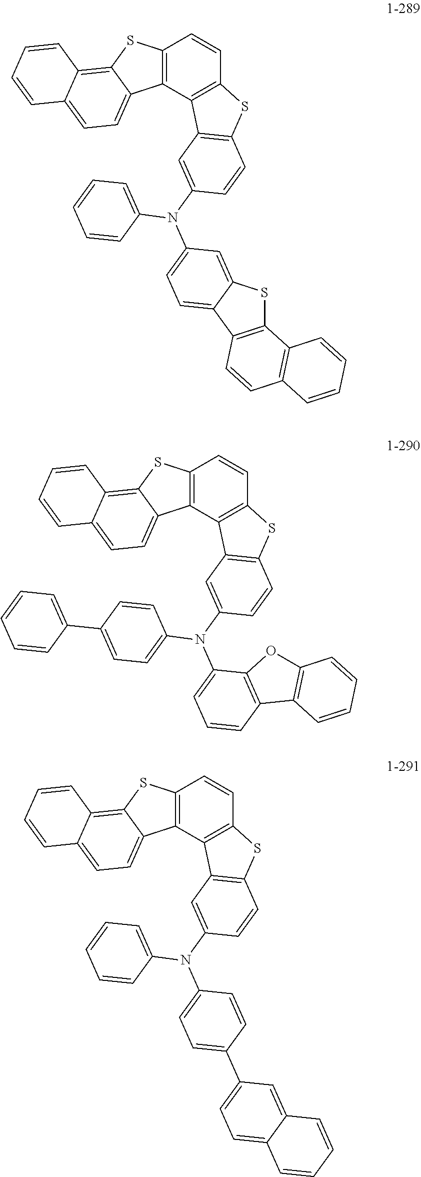 [SMILES: c1ccc(N(c2ccc3c(c2)sc2c4ccccc4ccc32)c2ccc3sc4ccc5sc6c7ccccc7ccc6c5c4c3c2)cc1 ; c1ccc(-c2ccc(N(c3ccc4sc5ccc6sc7c8ccccc8ccc7c6c5c4c3)c3cccc4c3oc3ccccc34)cc2)cc1 ; c1ccc(N(c2ccc(-c3ccc4ccccc4c3)cc2)c2ccc3sc4ccc5sc6c7ccccc7ccc6c5c4c3c2)cc1]

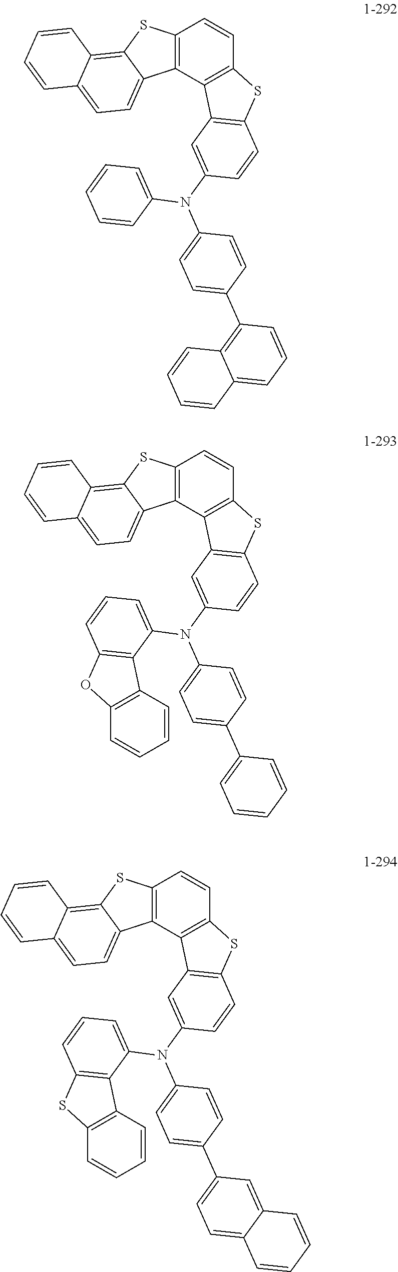

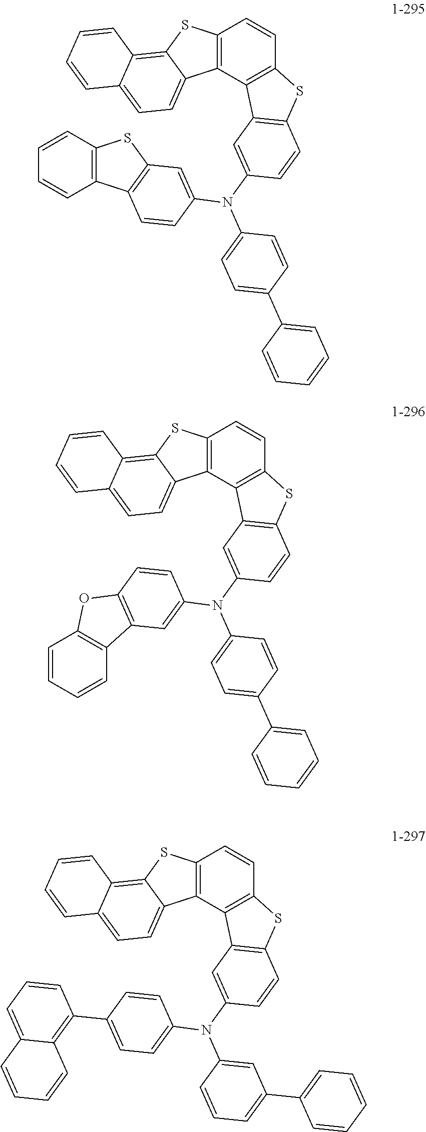 [SMILES: c1ccc(-c2ccc(N(c3ccc4c(c3)sc3ccccc34)c3ccc4sc5ccc6sc7c8ccccc8ccc7c6c5c4c3)cc2)cc1 ; c1ccc(-c2ccc(N(c3ccc4oc5ccccc5c4c3)c3ccc4sc5ccc6sc7c8ccccc8ccc7c6c5c4c3)cc2)cc1 ; c1ccc(-c2cccc(N(c3ccc(-c4cccc5ccccc45)cc3)c3ccc4sc5ccc6sc7c8ccccc8ccc7c6c5c4c3)c2)cc1]

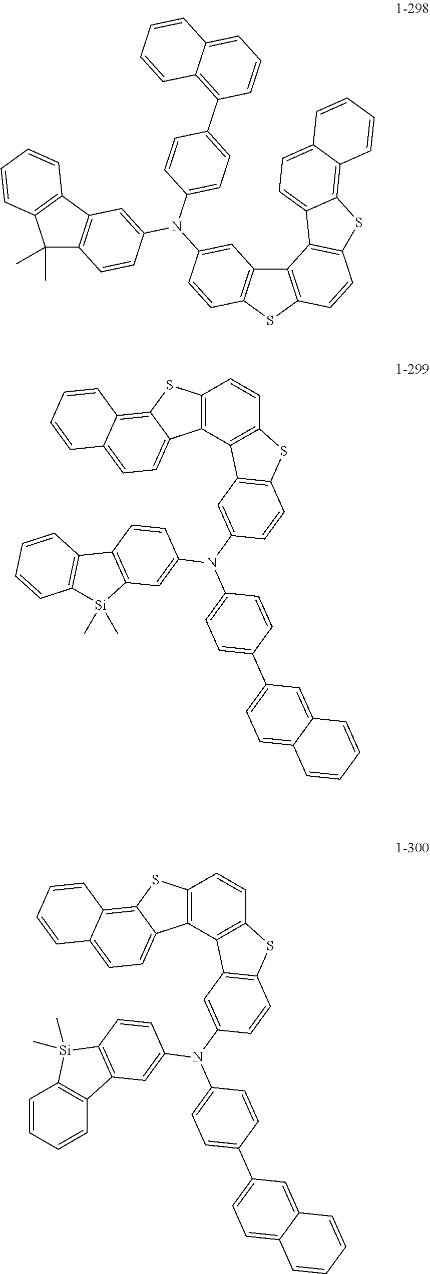 [SMILES: CC1(C)c2ccccc2-c2cc(N(c3ccc(-c4cccc5ccccc45)cc3)c3ccc4sc5ccc6sc7c8ccccc8ccc7c6c5c4c3)ccc21 ; C[Si]1(C)c2ccccc2-c2ccc(N(c3ccc(-c4ccc5ccccc5c4)cc3)c3ccc4sc5ccc6sc7c8ccccc8ccc7c6c5c4c3)cc21 ; C[Si]1(C)c2ccccc2-c2cc(N(c3ccc(-c4ccc5ccccc5c4)cc3)c3ccc4sc5ccc6sc7c8ccccc8ccc7c6c5c4c3)ccc21]

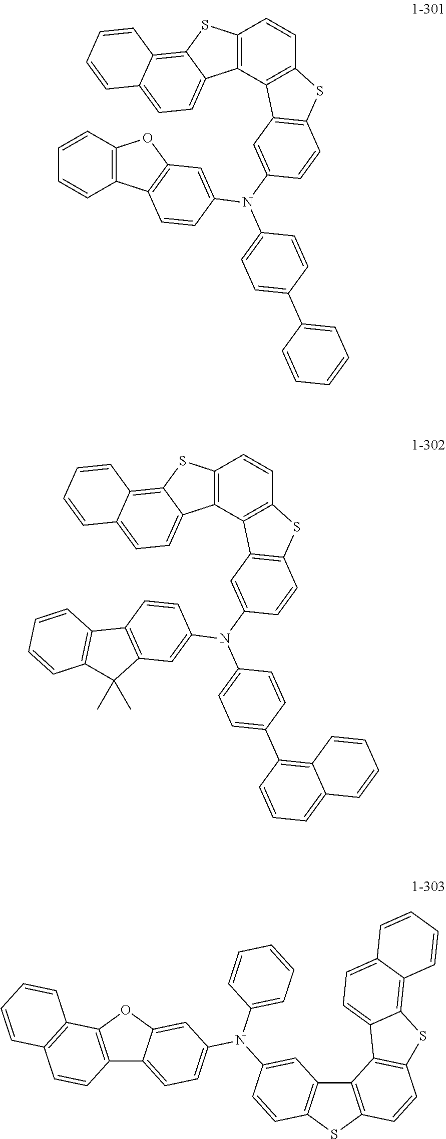 [SMILES: c1ccc(-c2ccc(N(c3ccc4c(c3)oc3ccccc34)c3ccc4sc5ccc6sc7c8ccccc8ccc7c6c5c4c3)cc2)cc1 ; CC1(C)c2ccccc2-c2ccc(N(c3ccc(-c4cccc5ccccc45)cc3)c3ccc4sc5ccc6sc7c8ccccc8ccc7c6c5c4c3)cc21 ; c1ccc(N(c2ccc3c(c2)oc2c4ccccc4ccc32)c2ccc3sc4ccc5sc6c7ccccc7ccc6c5c4c3c2)cc1]

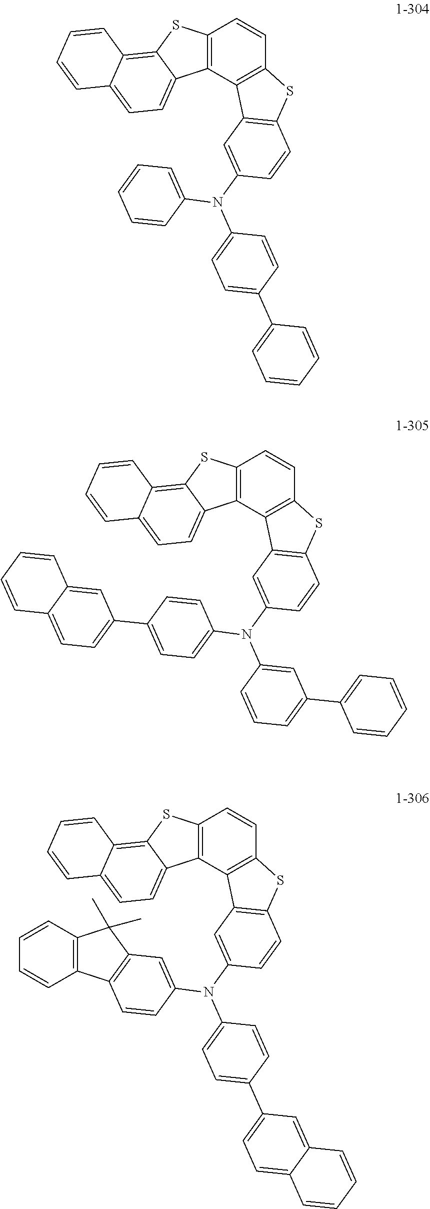 [SMILES: c1ccc(-c2ccc(N(c3ccccc3)c3ccc4sc5ccc6sc7c8ccccc8ccc7c6c5c4c3)cc2)cc1 ; c1ccc(-c2cccc(N(c3ccc(-c4ccc5ccccc5c4)cc3)c3ccc4sc5ccc6sc7c8ccccc8ccc7c6c5c4c3)c2)cc1 ; CC1(C)c2ccccc2-c2ccc(N(c3ccc(-c4ccc5ccccc5c4)cc3)c3ccc4sc5ccc6sc7c8ccccc8ccc7c6c5c4c3)cc21]

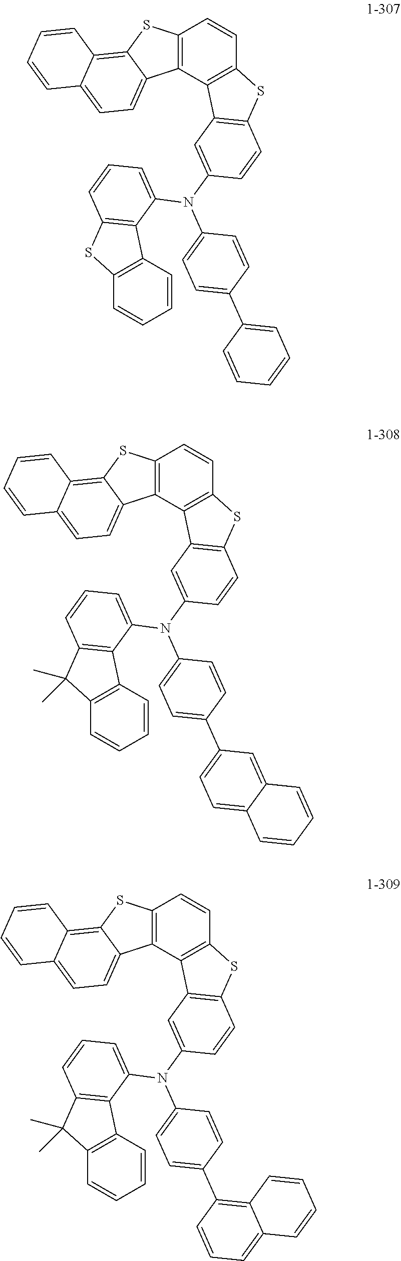 [SMILES: c1ccc(-c2ccc(N(c3ccc4sc5ccc6sc7c8ccccc8ccc7c6c5c4c3)c3cccc4sc5ccccc5c34)cc2)cc1 ; CC1(C)c2ccccc2-c2c(N(c3ccc(-c4ccc5ccccc5c4)cc3)c3ccc4sc5ccc6sc7c8ccccc8ccc7c6c5c4c3)cccc21 ; CC1(C)c2ccccc2-c2c(N(c3ccc(-c4cccc5ccccc45)cc3)c3ccc4sc5ccc6sc7c8ccccc8ccc7c6c5c4c3)cccc21]

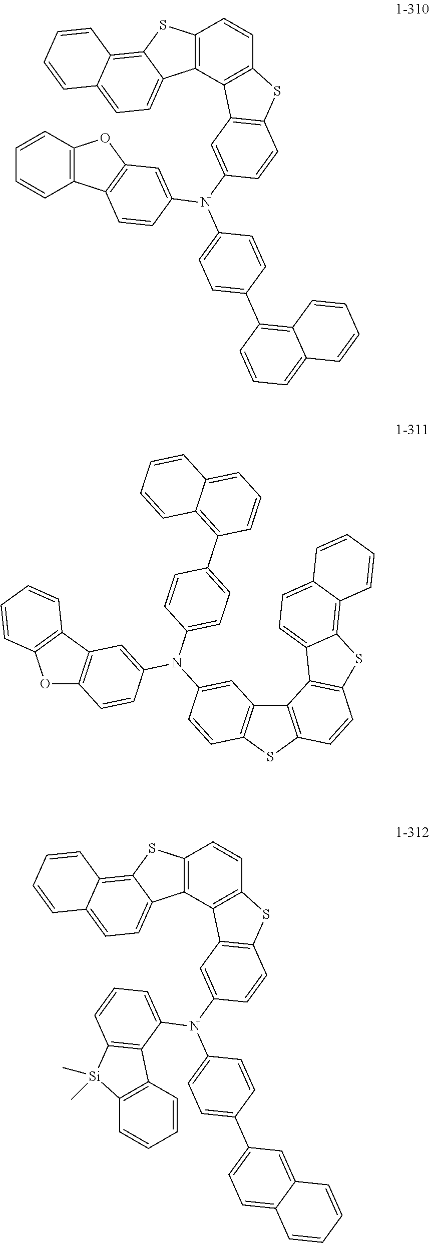 [SMILES: c1ccc2c(-c3ccc(N(c4ccc5c(c4)oc4ccccc45)c4ccc5sc6ccc7sc8c9ccccc9ccc8c7c6c5c4)cc3)cccc2c1 ; c1ccc2c(-c3ccc(N(c4ccc5oc6ccccc6c5c4)c4ccc5sc6ccc7sc8c9ccccc9ccc8c7c6c5c4)cc3)cccc2c1 ; C[Si]1(C)c2ccccc2-c2c(N(c3ccc(-c4ccc5ccccc5c4)cc3)c3ccc4sc5ccc6sc7c8ccccc8ccc7c6c5c4c3)cccc21]

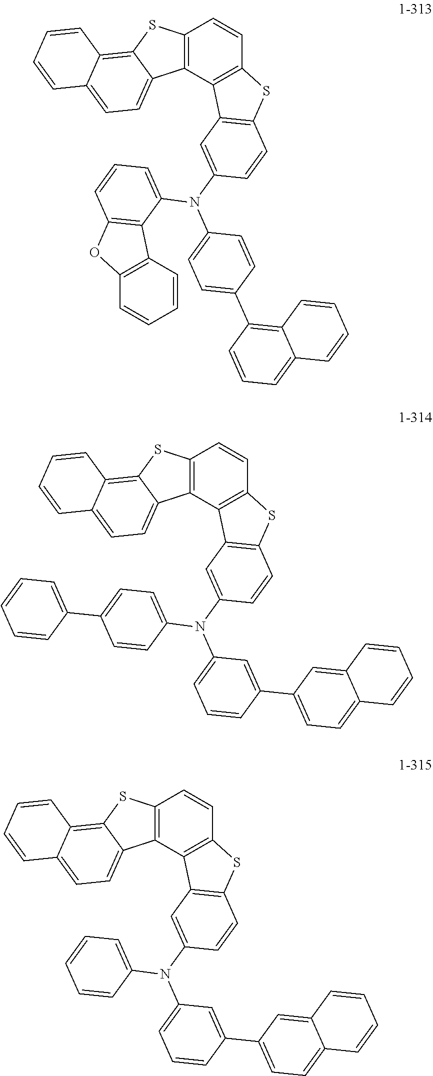 [SMILES: c1ccc2c(-c3ccc(N(c4ccc5sc6ccc7sc8c9ccccc9ccc8c7c6c5c4)c4cccc5oc6ccccc6c45)cc3)cccc2c1 ; c1ccc(-c2ccc(N(c3cccc(-c4ccc5ccccc5c4)c3)c3ccc4sc5ccc6sc7c8ccccc8ccc7c6c5c4c3)cc2)cc1 ; c1ccc(N(c2cccc(-c3ccc4ccccc4c3)c2)c2ccc3sc4ccc5sc6c7ccccc7ccc6c5c4c3c2)cc1]

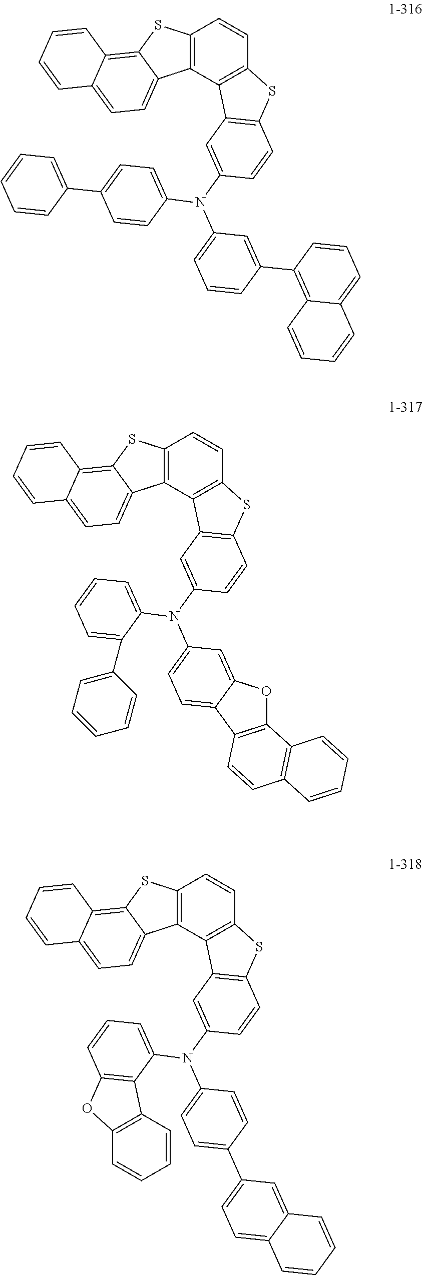 [SMILES: c1ccc(-c2ccc(N(c3cccc(-c4cccc5ccccc45)c3)c3ccc4sc5ccc6sc7c8ccccc8ccc7c6c5c4c3)cc2)cc1 ; c1ccc(-c2ccccc2N(c2ccc3c(c2)oc2c4ccccc4ccc32)c2ccc3sc4ccc5sc6c7ccccc7ccc6c5c4c3c2)cc1 ; c1ccc2cc(-c3ccc(N(c4ccc5sc6ccc7sc8c9ccccc9ccc8c7c6c5c4)c4cccc5oc6ccccc6c45)cc3)ccc2c1]

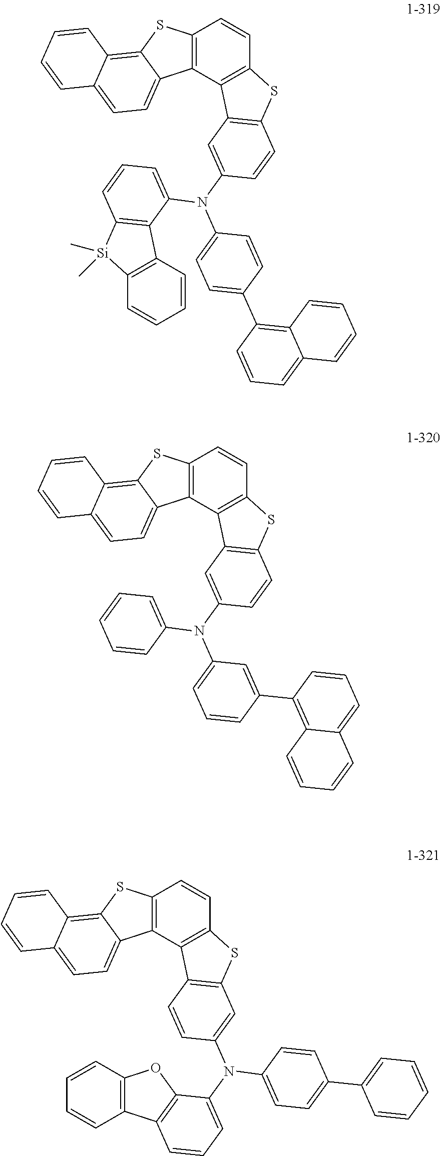 [SMILES: C[Si]1(C)c2ccccc2-c2c(N(c3ccc(-c4cccc5ccccc45)cc3)c3ccc4sc5ccc6sc7c8ccccc8ccc7c6c5c4c3)cccc21 ; c1ccc(N(c2cccc(-c3cccc4ccccc34)c2)c2ccc3sc4ccc5sc6c7ccccc7ccc6c5c4c3c2)cc1 ; c1ccc(-c2ccc(N(c3ccc4c(c3)sc3ccc5sc6c7ccccc7ccc6c5c34)c3cccc4c3oc3ccccc34)cc2)cc1]

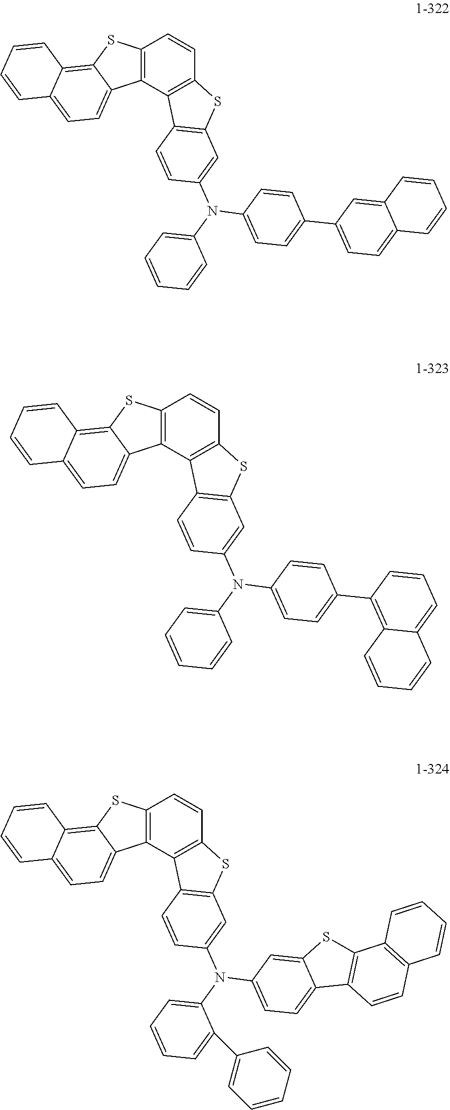 [SMILES: c1ccc(N(c2ccc(-c3ccc4ccccc4c3)cc2)c2ccc3c(c2)sc2ccc4sc5c6ccccc6ccc5c4c23)cc1 ; c1ccc(N(c2ccc(-c3cccc4ccccc34)cc2)c2ccc3c(c2)sc2ccc4sc5c6ccccc6ccc5c4c23)cc1 ; c1ccc(-c2ccccc2N(c2ccc3c(c2)sc2c4ccccc4ccc32)c2ccc3c(c2)sc2ccc4sc5c6ccccc6ccc5c4c23)cc1]

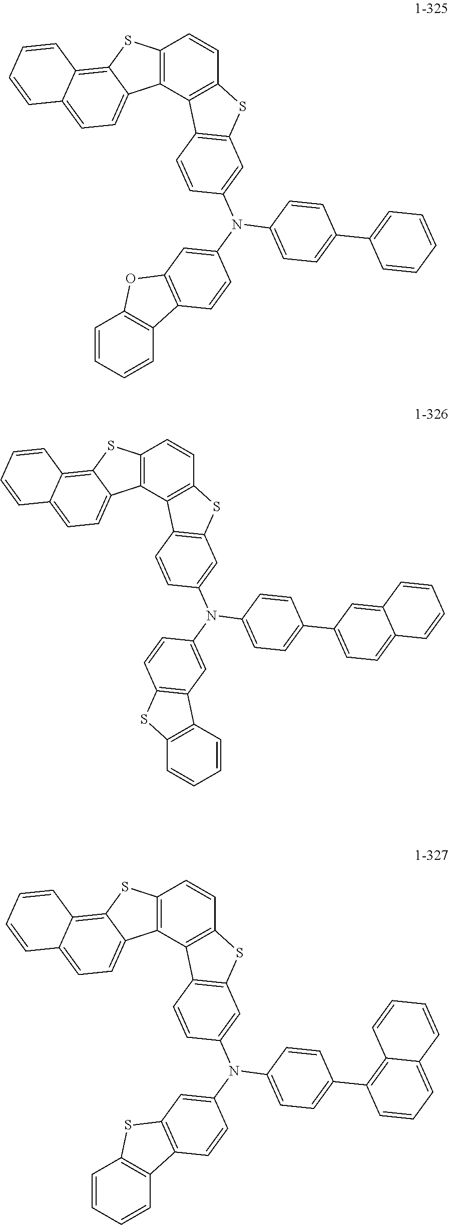 [SMILES: c1ccc(-c2ccc(N(c3ccc4c(c3)oc3ccccc34)c3ccc4c(c3)sc3ccc5sc6c7ccccc7ccc6c5c34)cc2)cc1 ; c1ccc2cc(-c3ccc(N(c4ccc5c(c4)sc4ccc6sc7c8ccccc8ccc7c6c45)c4ccc5sc6ccccc6c5c4)cc3)ccc2c1 ; c1ccc2c(-c3ccc(N(c4ccc5c(c4)sc4ccccc45)c4ccc5c(c4)sc4ccc6sc7c8ccccc8ccc7c6c45)cc3)cccc2c1]

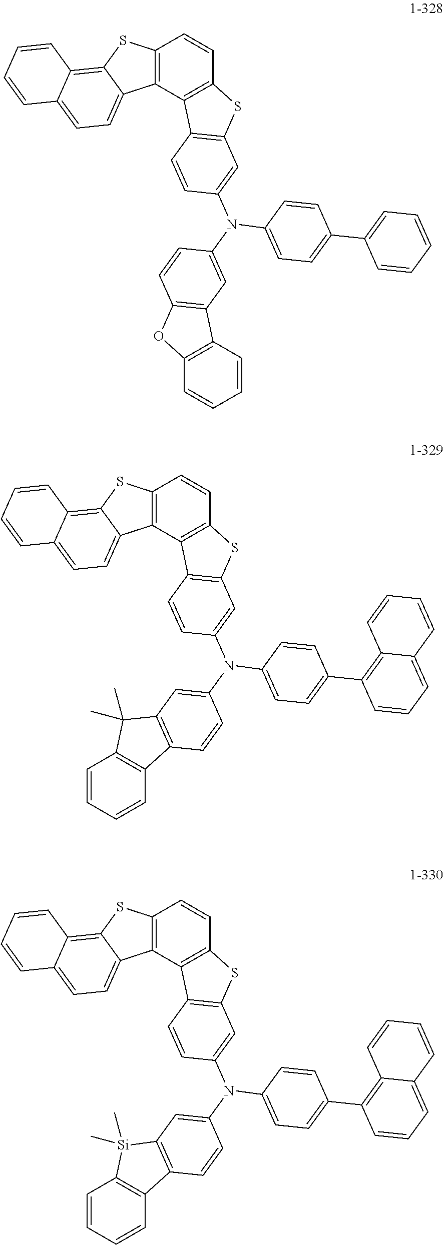 [SMILES: c1ccc(-c2ccc(N(c3ccc4c(c3)sc3ccc5sc6c7ccccc7ccc6c5c34)c3ccc4oc5ccccc5c4c3)cc2)cc1 ; CC1(C)c2ccccc2-c2ccc(N(c3ccc(-c4cccc5ccccc45)cc3)c3ccc4c(c3)sc3ccc5sc6c7ccccc7ccc6c5c34)cc21 ; C[Si]1(C)c2ccccc2-c2ccc(N(c3ccc(-c4cccc5ccccc45)cc3)c3ccc4c(c3)sc3ccc5sc6c7ccccc7ccc6c5c34)cc21]

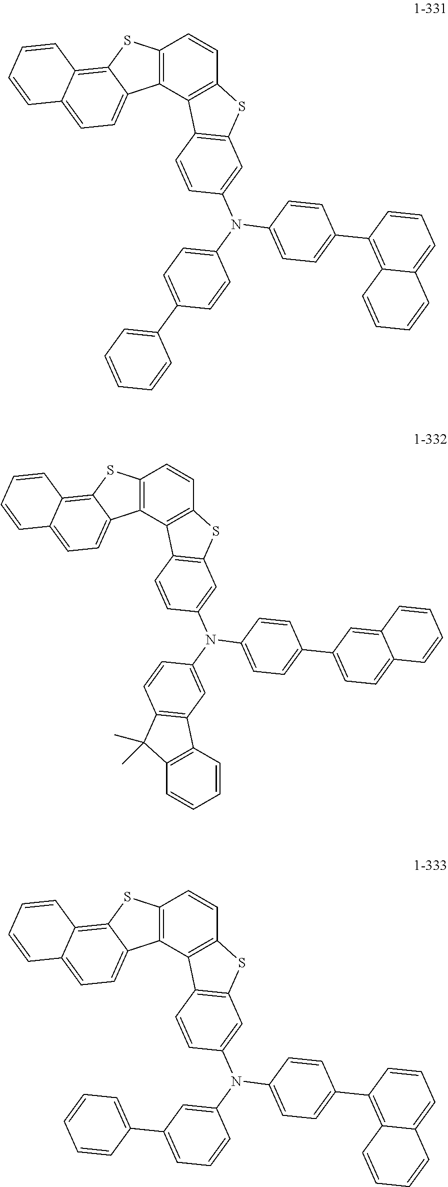 [SMILES: c1ccc(-c2ccc(N(c3ccc(-c4cccc5ccccc45)cc3)c3ccc4c(c3)sc3ccc5sc6c7ccccc7ccc6c5c34)cc2)cc1 ; CC1(C)c2ccccc2-c2cc(N(c3ccc(-c4ccc5ccccc5c4)cc3)c3ccc4c(c3)sc3ccc5sc6c7ccccc7ccc6c5c34)ccc21 ; c1ccc(-c2cccc(N(c3ccc(-c4cccc5ccccc45)cc3)c3ccc4c(c3)sc3ccc5sc6c7ccccc7ccc6c5c34)c2)cc1]

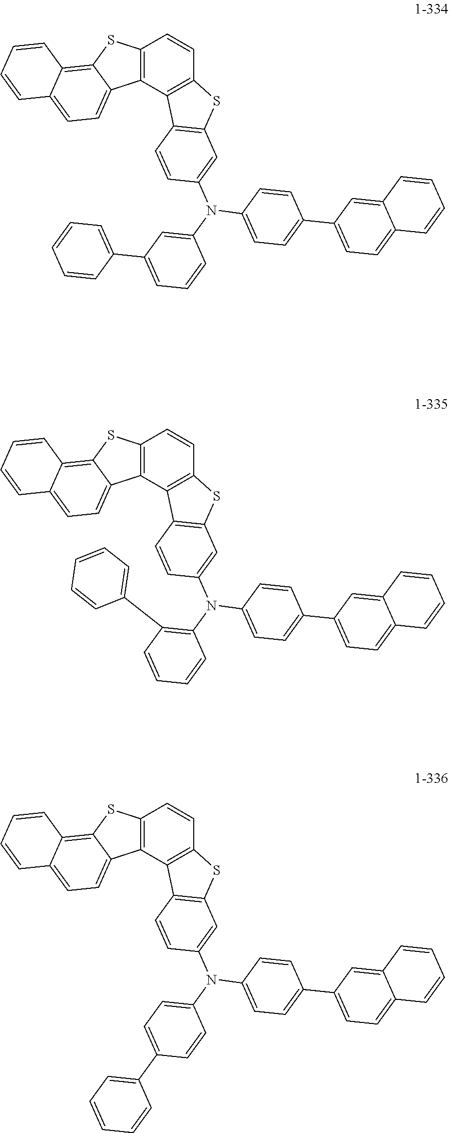 [SMILES: c1ccc(-c2cccc(N(c3ccc(-c4ccc5ccccc5c4)cc3)c3ccc4c(c3)sc3ccc5sc6c7ccccc7ccc6c5c34)c2)cc1 ; c1ccc(-c2ccccc2N(c2ccc(-c3ccc4ccccc4c3)cc2)c2ccc3c(c2)sc2ccc4sc5c6ccccc6ccc5c4c23)cc1 ; c1ccc(-c2ccc(N(c3ccc(-c4ccc5ccccc5c4)cc3)c3ccc4c(c3)sc3ccc5sc6c7ccccc7ccc6c5c34)cc2)cc1]

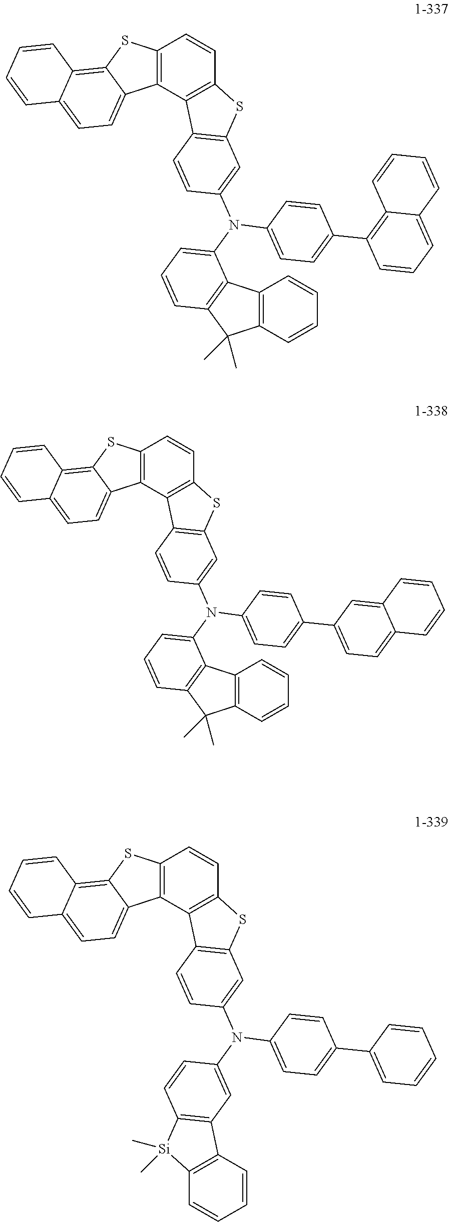 [SMILES: CC1(C)c2ccccc2-c2c(N(c3ccc(-c4cccc5ccccc45)cc3)c3ccc4c(c3)sc3ccc5sc6c7ccccc7ccc6c5c34)cccc21 ; CC1(C)c2ccccc2-c2c(N(c3ccc(-c4ccc5ccccc5c4)cc3)c3ccc4c(c3)sc3ccc5sc6c7ccccc7ccc6c5c34)cccc21 ; C[Si]1(C)c2ccccc2-c2cc(N(c3ccc(-c4ccccc4)cc3)c3ccc4c(c3)sc3ccc5sc6c7ccccc7ccc6c5c34)ccc21]

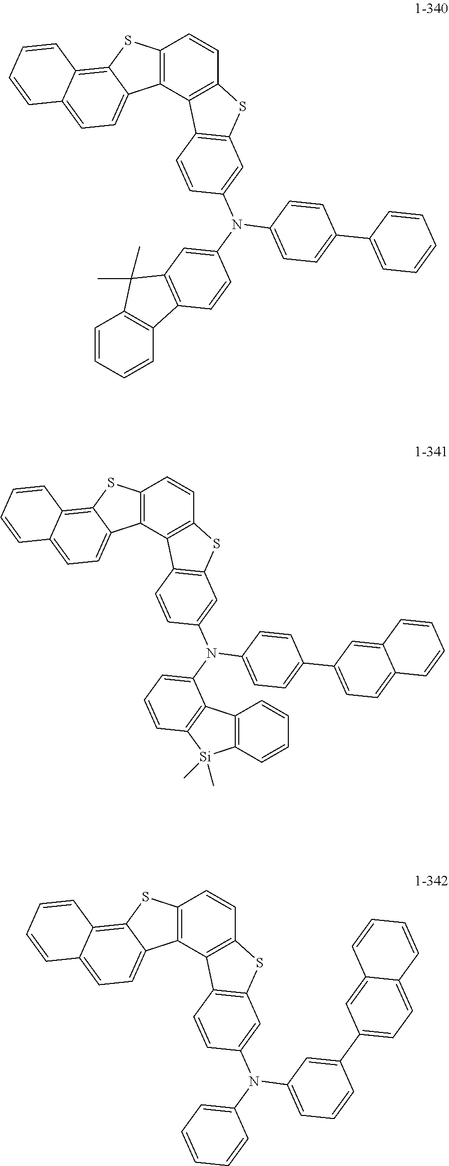 [SMILES: CC1(C)c2ccccc2-c2ccc(N(c3ccc(-c4ccccc4)cc3)c3ccc4c(c3)sc3ccc5sc6c7ccccc7ccc6c5c34)cc21 ; C[Si]1(C)c2ccccc2-c2c(N(c3ccc(-c4ccc5ccccc5c4)cc3)c3ccc4c(c3)sc3ccc5sc6c7ccccc7ccc6c5c34)cccc21 ; c1ccc(N(c2cccc(-c3ccc4ccccc4c3)c2)c2ccc3c(c2)sc2ccc4sc5c6ccccc6ccc5c4c23)cc1]

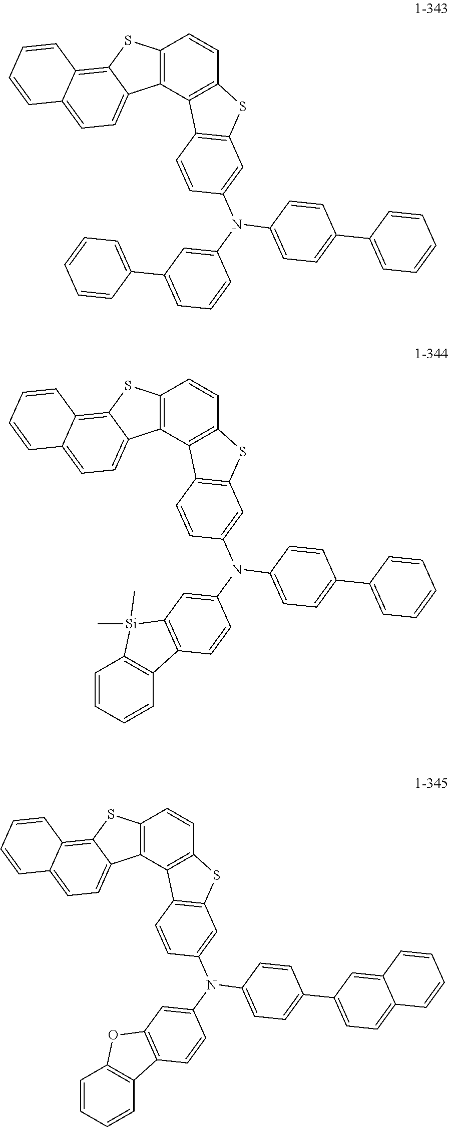 [SMILES: c1ccc(-c2ccc(N(c3cccc(-c4ccccc4)c3)c3ccc4c(c3)sc3ccc5sc6c7ccccc7ccc6c5c34)cc2)cc1 ; C[Si]1(C)c2ccccc2-c2ccc(N(c3ccc(-c4ccccc4)cc3)c3ccc4c(c3)sc3ccc5sc6c7ccccc7ccc6c5c34)cc21 ; c1ccc2cc(-c3ccc(N(c4ccc5c(c4)oc4ccccc45)c4ccc5c(c4)sc4ccc6sc7c8ccccc8ccc7c6c45)cc3)ccc2c1]

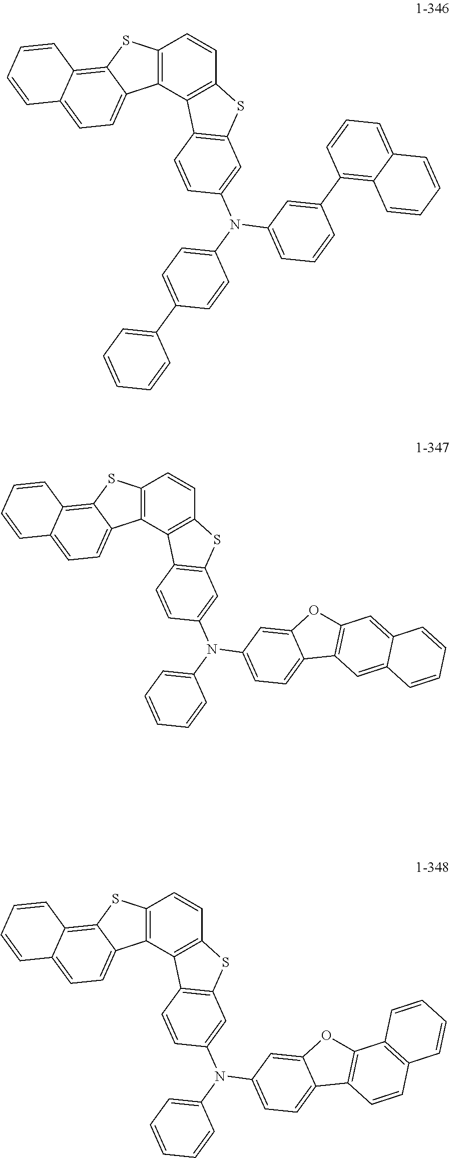 [SMILES: c1ccc(-c2ccc(N(c3cccc(-c4cccc5ccccc45)c3)c3ccc4c(c3)sc3ccc5sc6c7ccccc7ccc6c5c34)cc2)cc1 ; c1ccc(N(c2ccc3c(c2)oc2cc4ccccc4cc23)c2ccc3c(c2)sc2ccc4sc5c6ccccc6ccc5c4c23)cc1 ; c1ccc(N(c2ccc3c(c2)oc2c4ccccc4ccc32)c2ccc3c(c2)sc2ccc4sc5c6ccccc6ccc5c4c23)cc1]

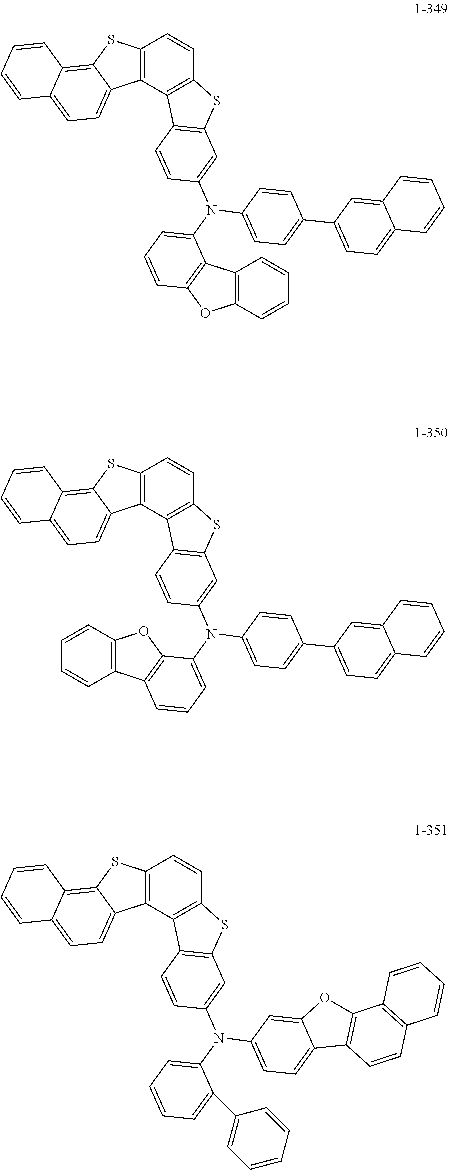 [SMILES: c1ccc2cc(-c3ccc(N(c4ccc5c(c4)sc4ccc6sc7c8ccccc8ccc7c6c45)c4cccc5oc6ccccc6c45)cc3)ccc2c1 ; c1ccc2cc(-c3ccc(N(c4ccc5c(c4)sc4ccc6sc7c8ccccc8ccc7c6c45)c4cccc5c4oc4ccccc45)cc3)ccc2c1 ; c1ccc(-c2ccccc2N(c2ccc3c(c2)oc2c4ccccc4ccc32)c2ccc3c(c2)sc2ccc4sc5c6ccccc6ccc5c4c23)cc1]

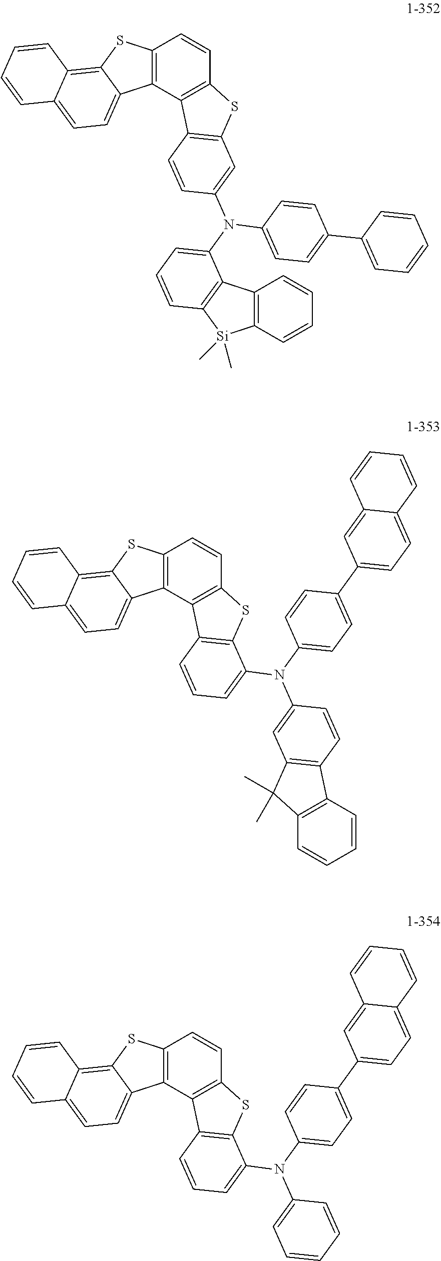 [SMILES: C[Si]1(C)c2ccccc2-c2c(N(c3ccc(-c4ccccc4)cc3)c3ccc4c(c3)sc3ccc5sc6c7ccccc7ccc6c5c34)cccc21 ; CC1(C)c2ccccc2-c2ccc(N(c3ccc(-c4ccc5ccccc5c4)cc3)c3cccc4c3sc3ccc5sc6c7ccccc7ccc6c5c34)cc21 ; c1ccc(N(c2ccc(-c3ccc4ccccc4c3)cc2)c2cccc3c2sc2ccc4sc5c6ccccc6ccc5c4c23)cc1]

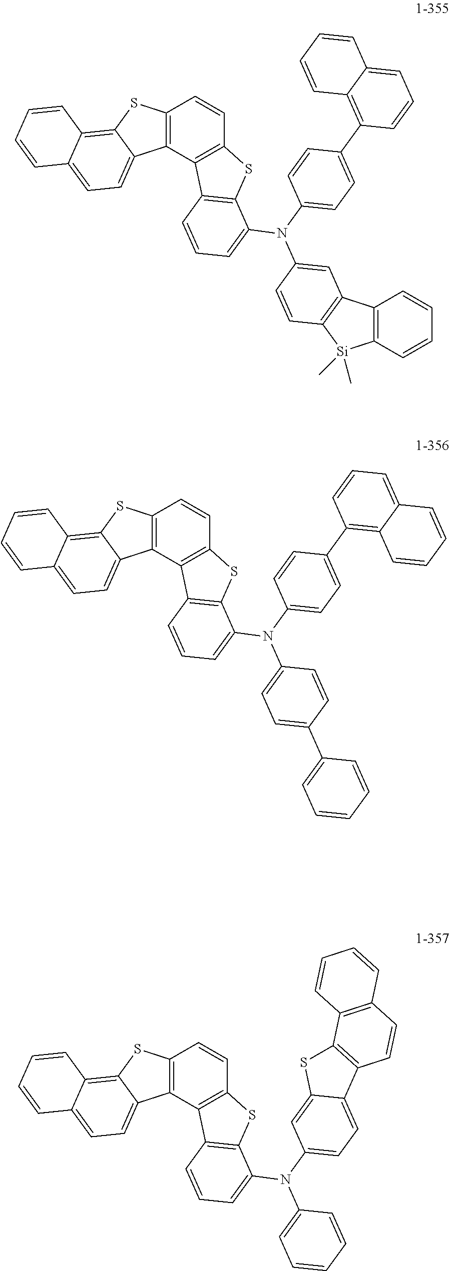 [SMILES: C[Si]1(C)c2ccccc2-c2cc(N(c3ccc(-c4cccc5ccccc45)cc3)c3cccc4c3sc3ccc5sc6c7ccccc7ccc6c5c34)ccc21 ; c1ccc(-c2ccc(N(c3ccc(-c4cccc5ccccc45)cc3)c3cccc4c3sc3ccc5sc6c7ccccc7ccc6c5c34)cc2)cc1 ; c1ccc(N(c2ccc3c(c2)sc2c4ccccc4ccc32)c2cccc3c2sc2ccc4sc5c6ccccc6ccc5c4c23)cc1]

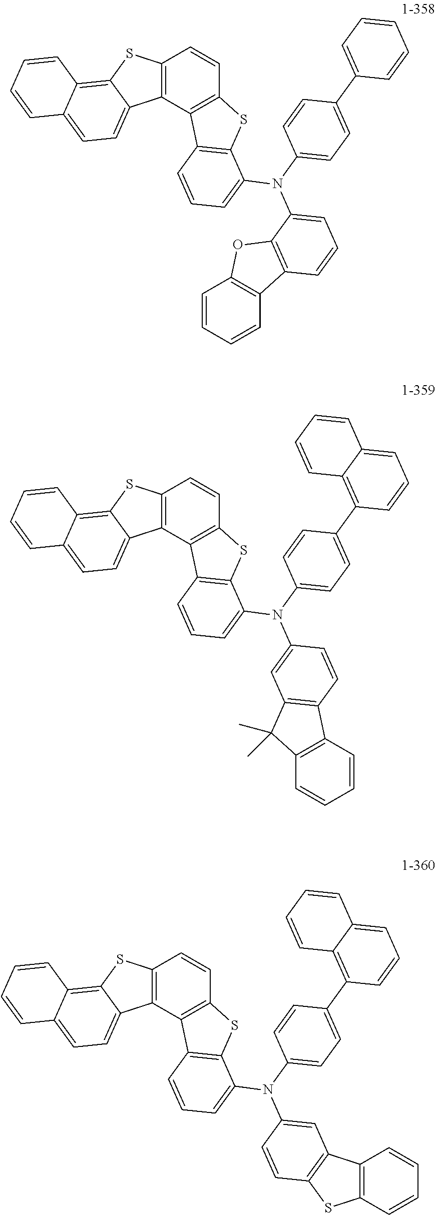 [SMILES: c1ccc(-c2ccc(N(c3cccc4c3oc3ccccc34)c3cccc4c3sc3ccc5sc6c7ccccc7ccc6c5c34)cc2)cc1 ; CC1(C)c2ccccc2-c2ccc(N(c3ccc(-c4cccc5ccccc45)cc3)c3cccc4c3sc3ccc5sc6c7ccccc7ccc6c5c34)cc21 ; c1ccc2c(-c3ccc(N(c4ccc5sc6ccccc6c5c4)c4cccc5c4sc4ccc6sc7c8ccccc8ccc7c6c45)cc3)cccc2c1]

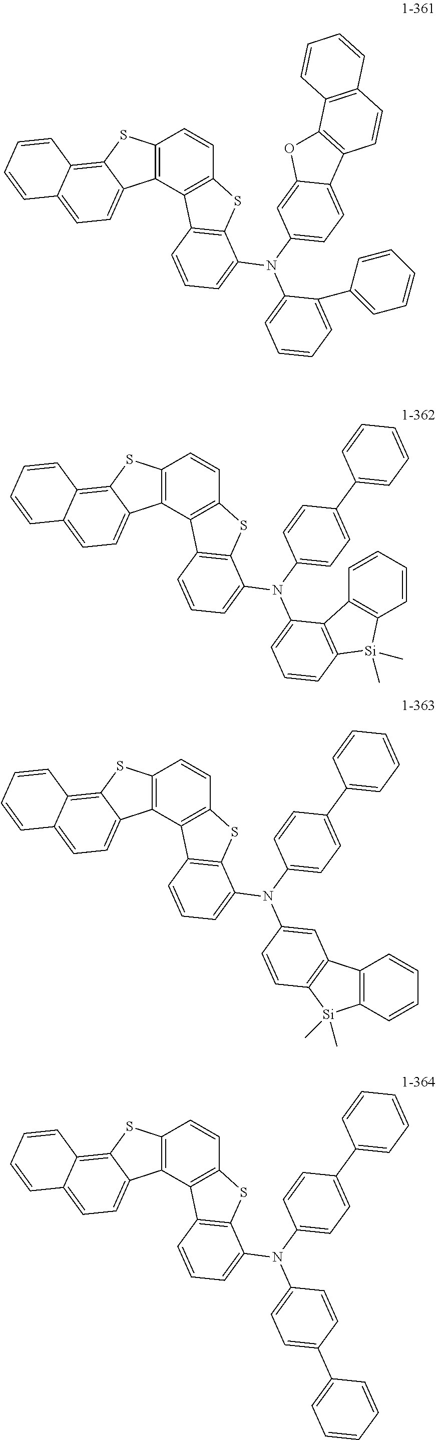 [SMILES: c1ccc(-c2ccccc2N(c2ccc3c(c2)oc2c4ccccc4ccc32)c2cccc3c2sc2ccc4sc5c6ccccc6ccc5c4c23)cc1 ; C[Si]1(C)c2ccccc2-c2c(N(c3ccc(-c4ccccc4)cc3)c3cccc4c3sc3ccc5sc6c7ccccc7ccc6c5c34)cccc21 ; C[Si]1(C)c2ccccc2-c2cc(N(c3ccc(-c4ccccc4)cc3)c3cccc4c3sc3ccc5sc6c7ccccc7ccc6c5c34)ccc21 ; c1ccc(-c2ccc(N(c3ccc(-c4ccccc4)cc3)c3cccc4c3sc3ccc5sc6c7ccccc7ccc6c5c34)cc2)cc1]

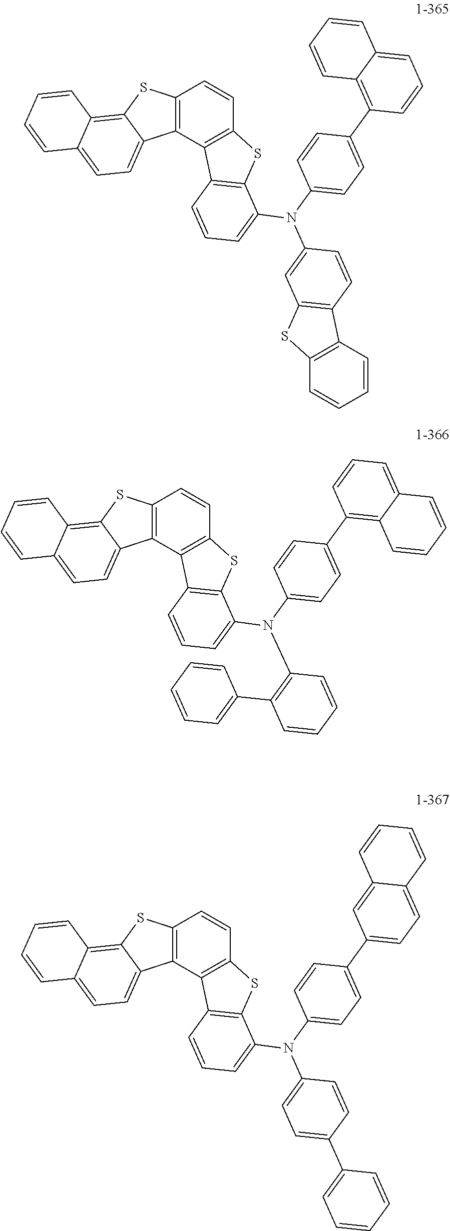 [SMILES: c1ccc2c(-c3ccc(N(c4ccc5c(c4)sc4ccccc45)c4cccc5c4sc4ccc6sc7c8ccccc8ccc7c6c45)cc3)cccc2c1 ; c1ccc(-c2ccccc2N(c2ccc(-c3cccc4ccccc34)cc2)c2cccc3c2sc2ccc4sc5c6ccccc6ccc5c4c23)cc1 ; c1ccc(-c2ccc(N(c3ccc(-c4ccc5ccccc5c4)cc3)c3cccc4c3sc3ccc5sc6c7ccccc7ccc6c5c34)cc2)cc1]

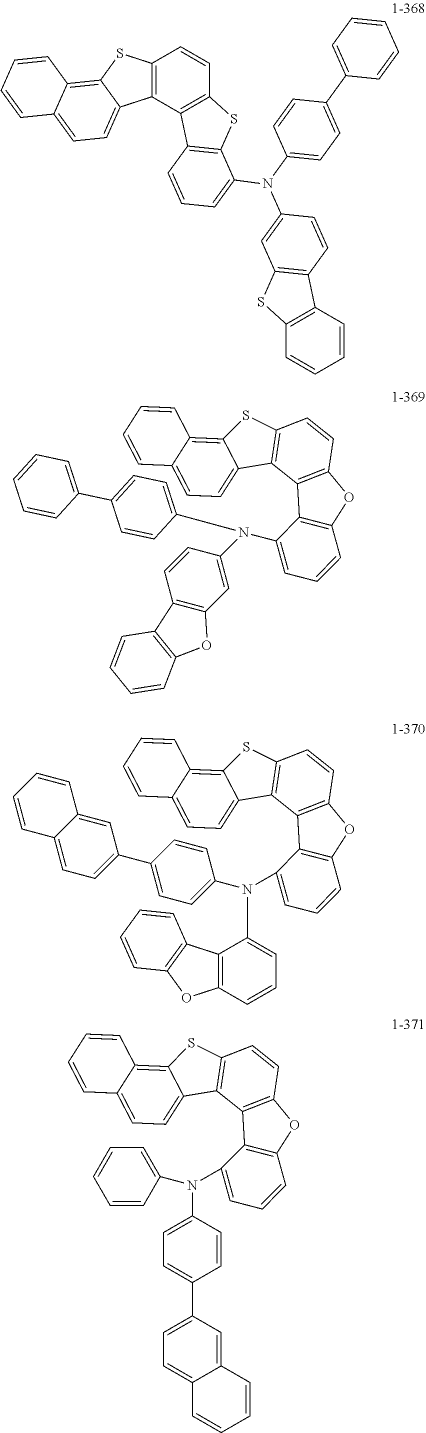

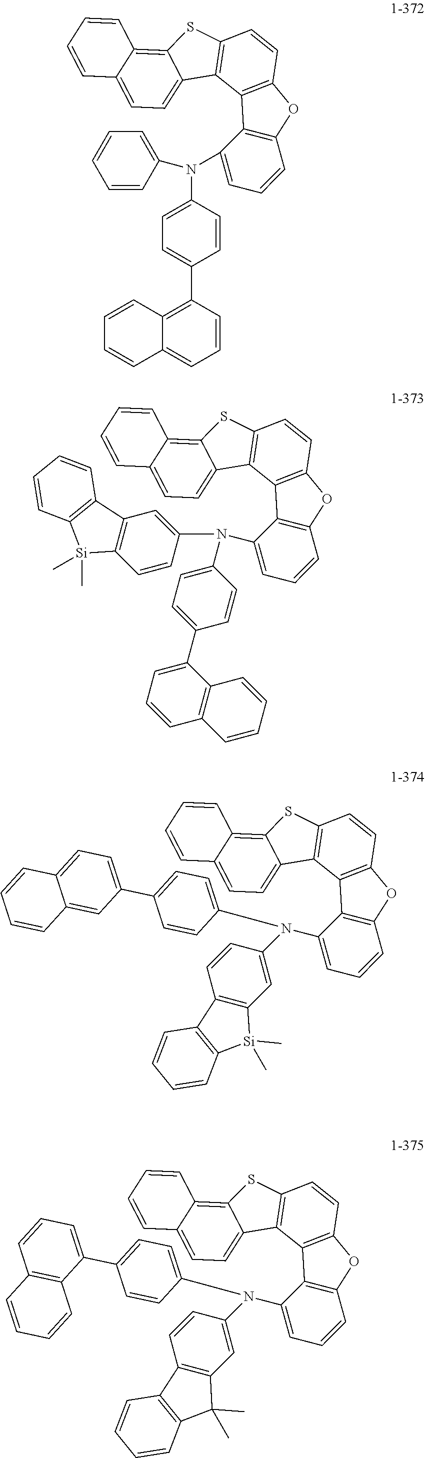

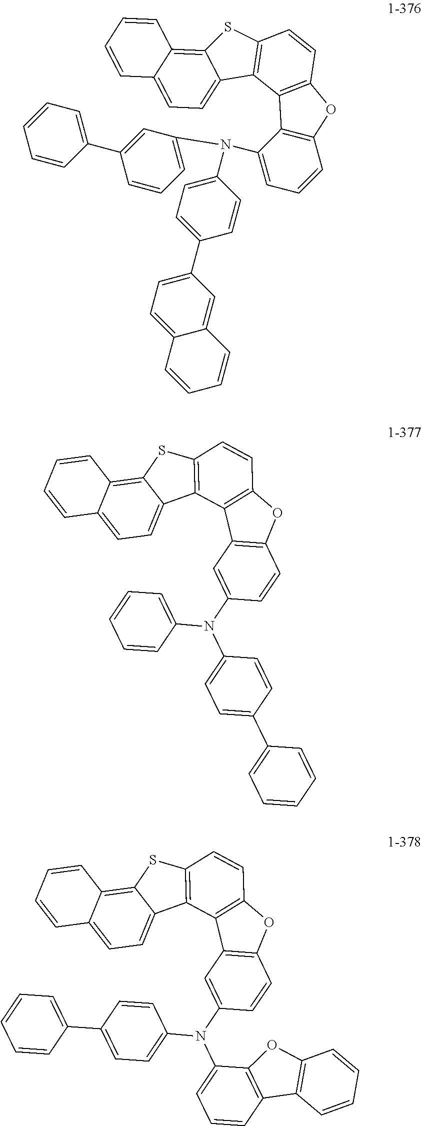 [SMILES: c1ccc(-c2cccc(N(c3ccc(-c4ccc5ccccc5c4)cc3)c3cccc4oc5ccc6sc7c8ccccc8ccc7c6c5c34)c2)cc1 ; c1ccc(-c2ccc(N(c3ccccc3)c3ccc4oc5ccc6sc7c8ccccc8ccc7c6c5c4c3)cc2)cc1 ; c1ccc(-c2ccc(N(c3ccc4oc5ccc6sc7c8ccccc8ccc7c6c5c4c3)c3cccc4c3oc3ccccc34)cc2)cc1]

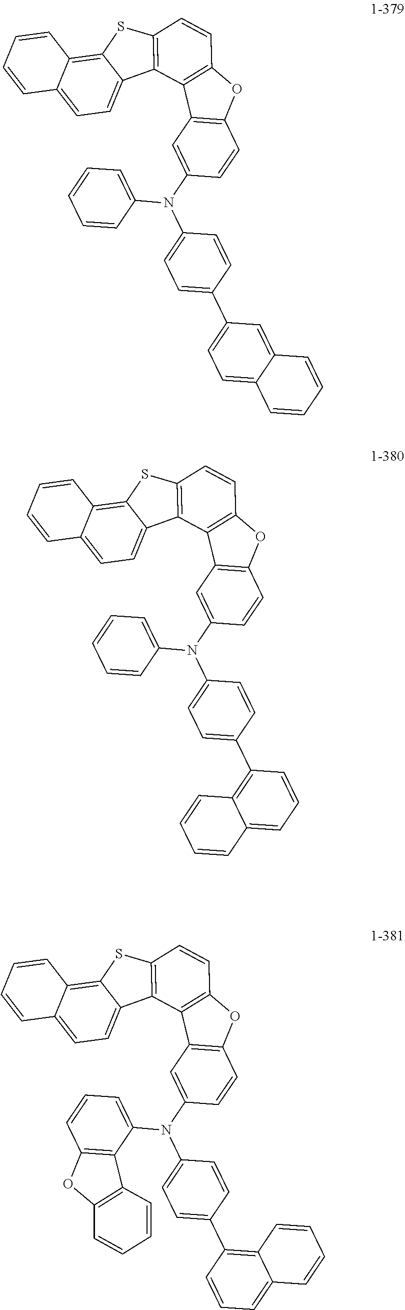 [SMILES: c1ccc(N(c2ccc(-c3ccc4ccccc4c3)cc2)c2ccc3oc4ccc5sc6c7ccccc7ccc6c5c4c3c2)cc1 ; c1ccc(N(c2ccc(-c3cccc4ccccc34)cc2)c2ccc3oc4ccc5sc6c7ccccc7ccc6c5c4c3c2)cc1 ; c1ccc2c(-c3ccc(N(c4ccc5oc6ccc7sc8c9ccccc9ccc8c7c6c5c4)c4cccc5oc6ccccc6c45)cc3)cccc2c1]

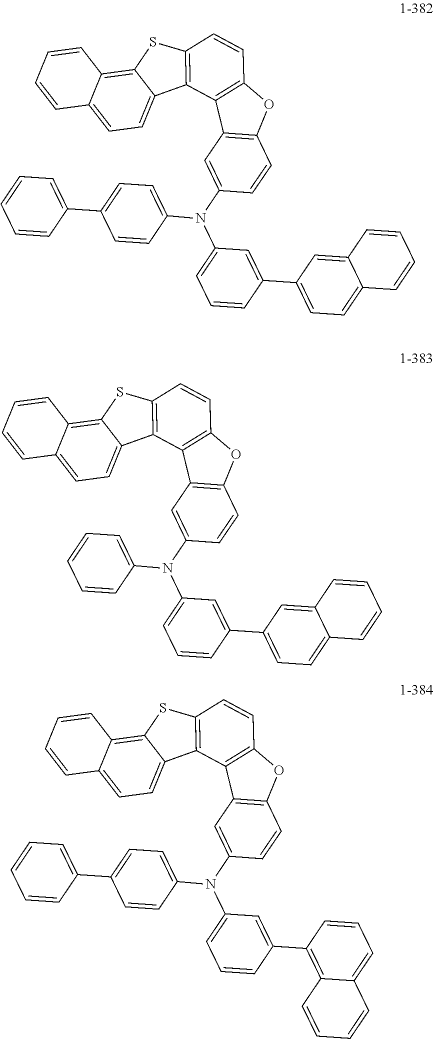

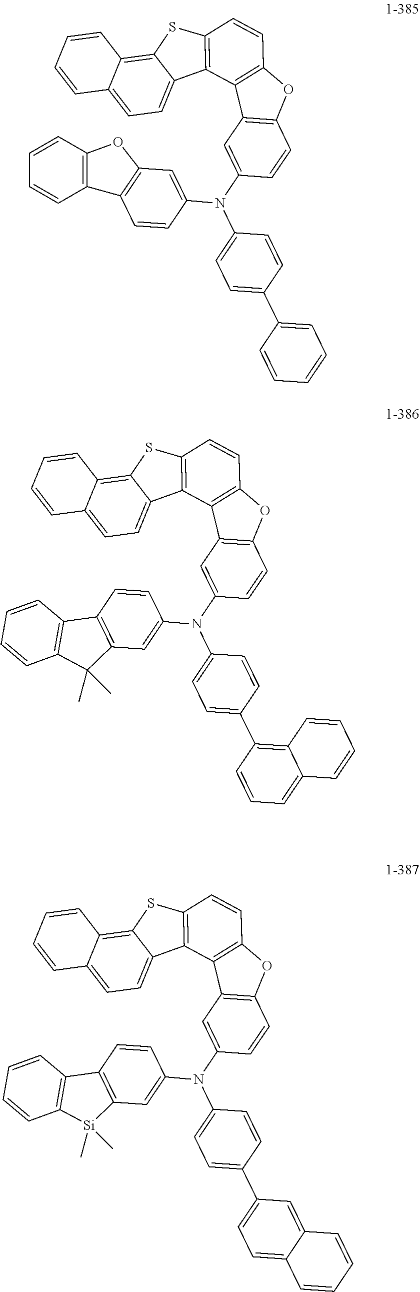 [SMILES: c1ccc(-c2ccc(N(c3ccc4c(c3)oc3ccccc34)c3ccc4oc5ccc6sc7c8ccccc8ccc7c6c5c4c3)cc2)cc1 ; CC1(C)c2ccccc2-c2ccc(N(c3ccc(-c4cccc5ccccc45)cc3)c3ccc4oc5ccc6sc7c8ccccc8ccc7c6c5c4c3)cc21 ; C[Si]1(C)c2ccccc2-c2ccc(N(c3ccc(-c4ccc5ccccc5c4)cc3)c3ccc4oc5ccc6sc7c8ccccc8ccc7c6c5c4c3)cc21]

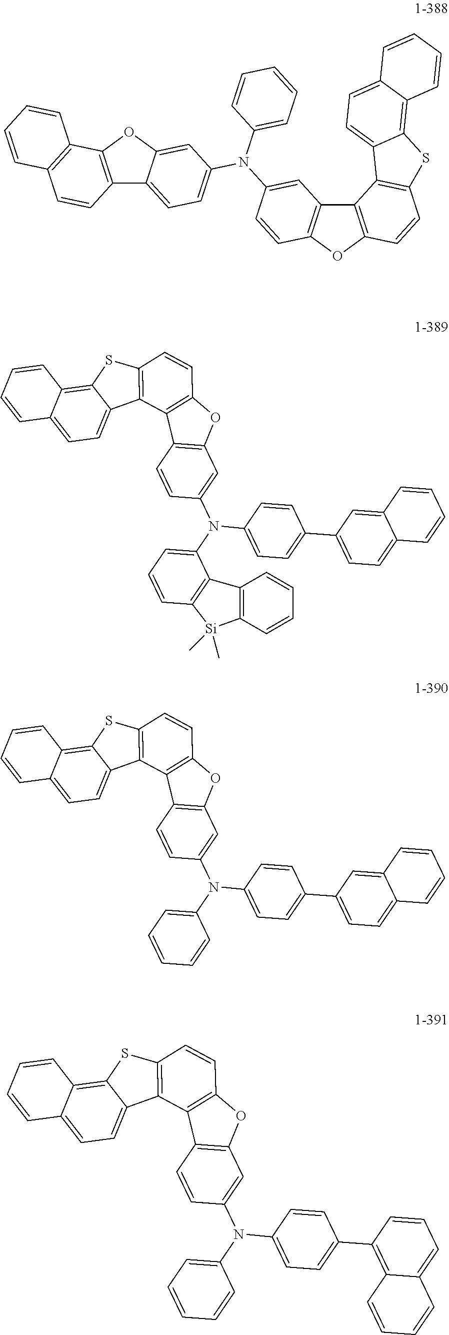 [SMILES: c1ccc(N(c2ccc3c(c2)oc2c4ccccc4ccc32)c2ccc3oc4ccc5sc6c7ccccc7ccc6c5c4c3c2)cc1 ; C[Si]1(C)c2ccccc2-c2c(N(c3ccc(-c4ccc5ccccc5c4)cc3)c3ccc4c(c3)oc3ccc5sc6c7ccccc7ccc6c5c34)cccc21 ; c1ccc(N(c2ccc(-c3ccc4ccccc4c3)cc2)c2ccc3c(c2)oc2ccc4sc5c6ccccc6ccc5c4c23)cc1 ; c1ccc(N(c2ccc(-c3cccc4ccccc34)cc2)c2ccc3c(c2)oc2ccc4sc5c6ccccc6ccc5c4c23)cc1]

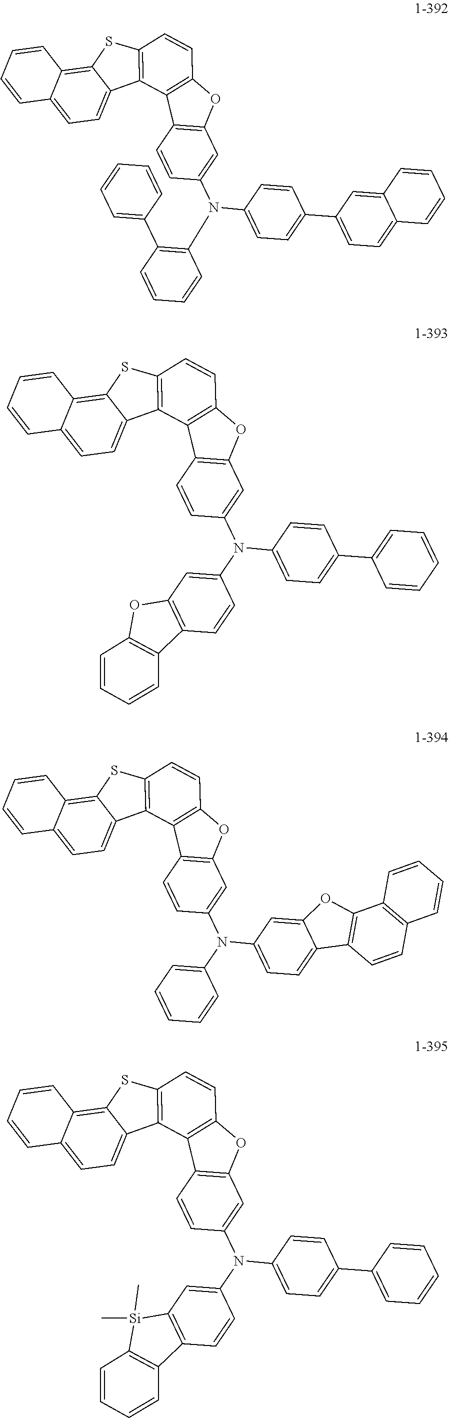 [SMILES: c1ccc(-c2ccccc2N(c2ccc(-c3ccc4ccccc4c3)cc2)c2ccc3c(c2)oc2ccc4sc5c6ccccc6ccc5c4c23)cc1 ; c1ccc(-c2ccc(N(c3ccc4c(c3)oc3ccccc34)c3ccc4c(c3)oc3ccc5sc6c7ccccc7ccc6c5c34)cc2)cc1 ; c1ccc(N(c2ccc3c(c2)oc2c4ccccc4ccc32)c2ccc3c(c2)oc2ccc4sc5c6ccccc6ccc5c4c23)cc1 ; C[Si]1(C)c2ccccc2-c2ccc(N(c3ccc(-c4ccccc4)cc3)c3ccc4c(c3)oc3ccc5sc6c7ccccc7ccc6c5c34)cc21]

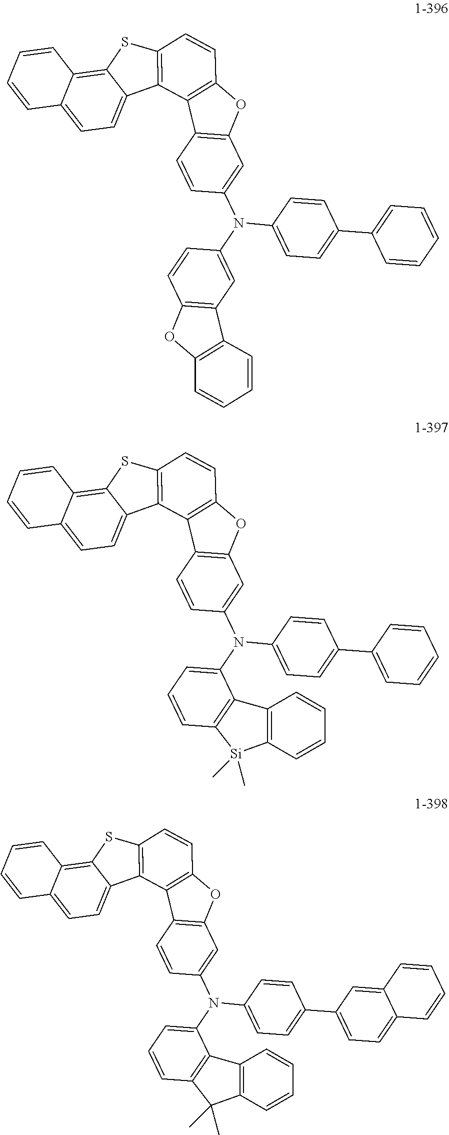 [SMILES: c1ccc(-c2ccc(N(c3ccc4c(c3)oc3ccc5sc6c7ccccc7ccc6c5c34)c3ccc4oc5ccccc5c4c3)cc2)cc1 ; C[Si]1(C)c2ccccc2-c2c(N(c3ccc(-c4ccccc4)cc3)c3ccc4c(c3)oc3ccc5sc6c7ccccc7ccc6c5c34)cccc21 ; CC1(C)c2ccccc2-c2c(N(c3ccc(-c4ccc5ccccc5c4)cc3)c3ccc4c(c3)oc3ccc5sc6c7ccccc7ccc6c5c34)cccc21]

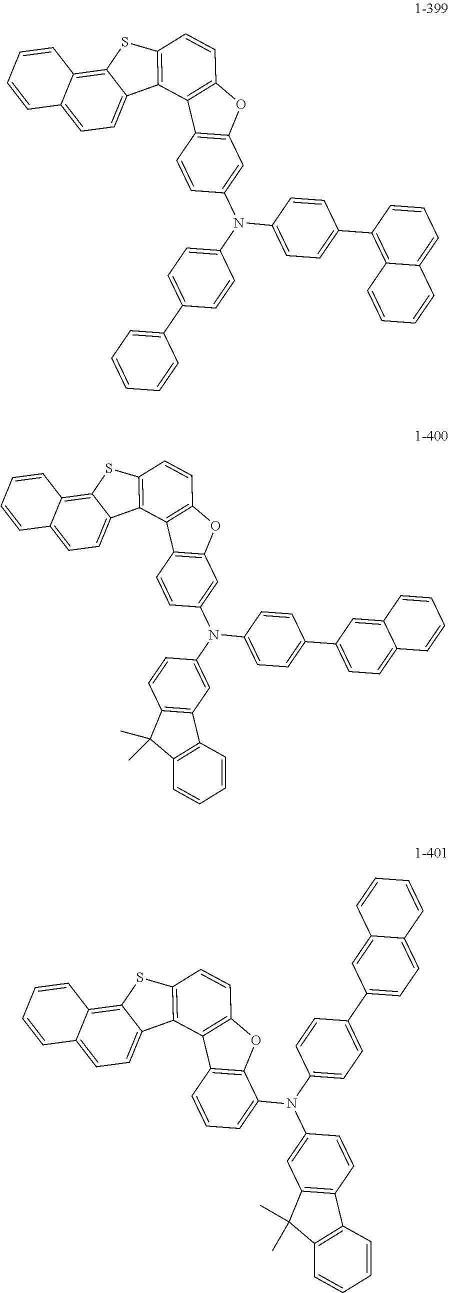 [SMILES: c1ccc(-c2ccc(N(c3ccc(-c4cccc5ccccc45)cc3)c3ccc4c(c3)oc3ccc5sc6c7ccccc7ccc6c5c34)cc2)cc1 ; CC1(C)c2ccccc2-c2cc(N(c3ccc(-c4ccc5ccccc5c4)cc3)c3ccc4c(c3)oc3ccc5sc6c7ccccc7ccc6c5c34)ccc21 ; CC1(C)c2ccccc2-c2ccc(N(c3ccc(-c4ccc5ccccc5c4)cc3)c3cccc4c3oc3ccc5sc6c7ccccc7ccc6c5c34)cc21]

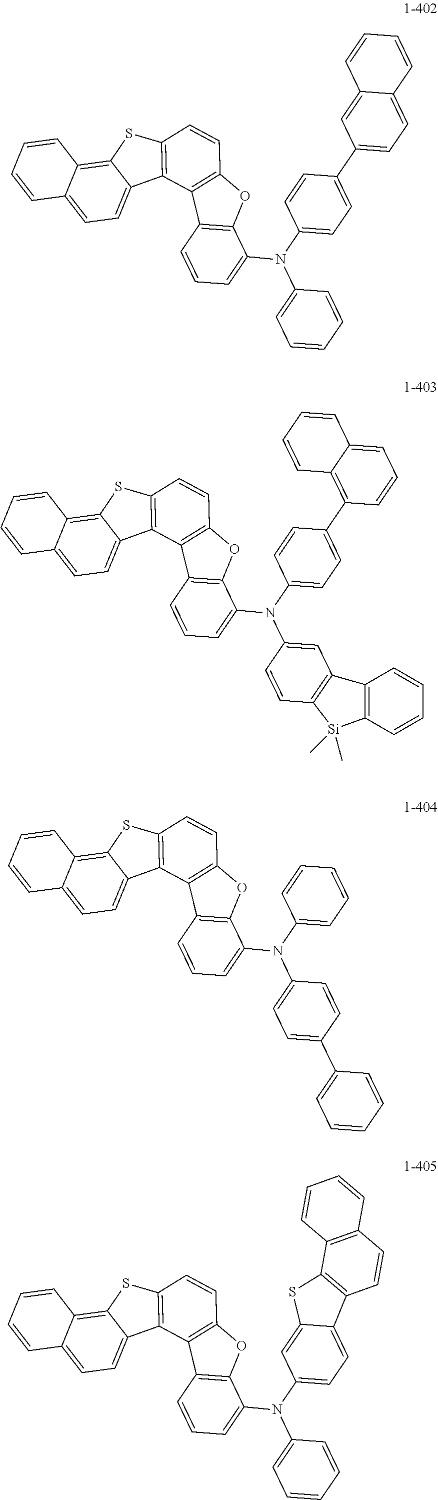 [SMILES: c1ccc(N(c2ccc(-c3ccc4ccccc4c3)cc2)c2cccc3c2oc2ccc4sc5c6ccccc6ccc5c4c23)cc1 ; C[Si]1(C)c2ccccc2-c2cc(N(c3ccc(-c4cccc5ccccc45)cc3)c3cccc4c3oc3ccc5sc6c7ccccc7ccc6c5c34)ccc21 ; c1ccc(-c2ccc(N(c3ccccc3)c3cccc4c3oc3ccc5sc6c7ccccc7ccc6c5c34)cc2)cc1 ; c1ccc(N(c2ccc3c(c2)sc2c4ccccc4ccc32)c2cccc3c2oc2ccc4sc5c6ccccc6ccc5c4c23)cc1]

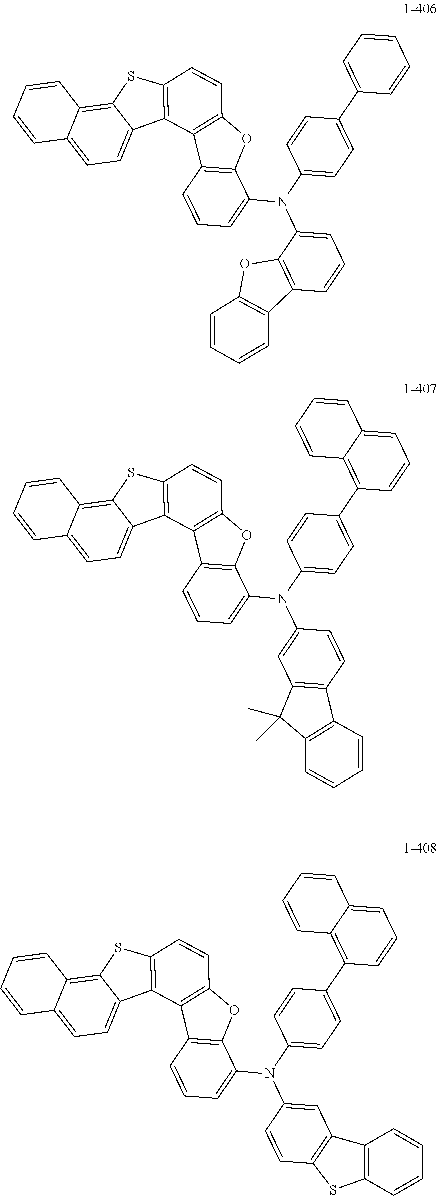 [SMILES: c1ccc(-c2ccc(N(c3cccc4c3oc3ccccc34)c3cccc4c3oc3ccc5sc6c7ccccc7ccc6c5c34)cc2)cc1 ; CC1(C)c2ccccc2-c2ccc(N(c3ccc(-c4cccc5ccccc45)cc3)c3cccc4c3oc3ccc5sc6c7ccccc7ccc6c5c34)cc21 ; c1ccc2c(-c3ccc(N(c4ccc5sc6ccccc6c5c4)c4cccc5c4oc4ccc6sc7c8ccccc8ccc7c6c45)cc3)cccc2c1]

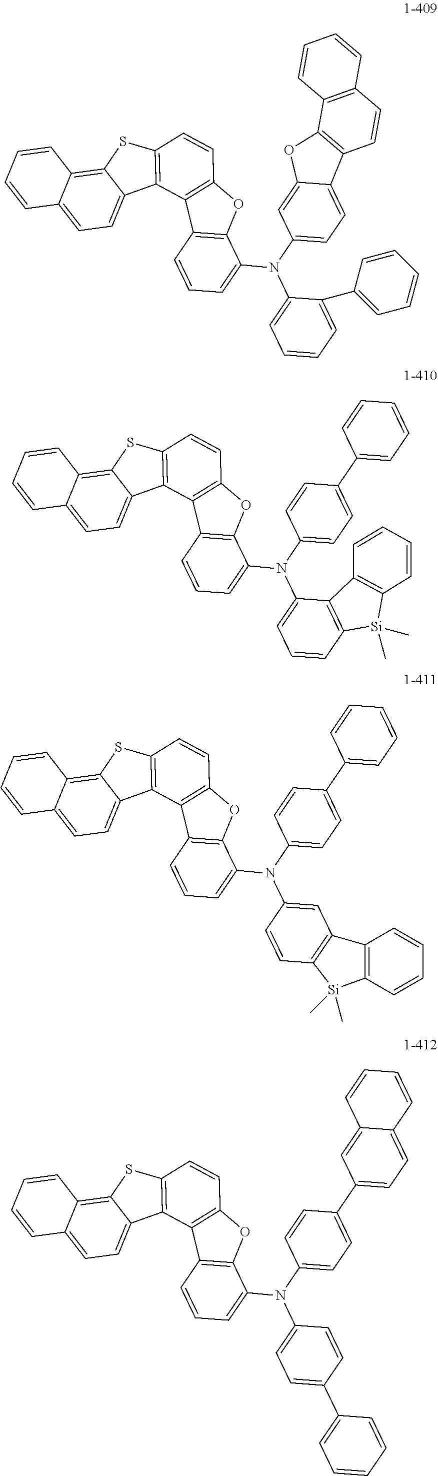 [SMILES: c1ccc(-c2ccccc2N(c2ccc3c(c2)oc2c4ccccc4ccc32)c2cccc3c2oc2ccc4sc5c6ccccc6ccc5c4c23)cc1 ; C[Si]1(C)c2ccccc2-c2c(N(c3ccc(-c4ccccc4)cc3)c3cccc4c3oc3ccc5sc6c7ccccc7ccc6c5c34)cccc21 ; C[Si]1(C)c2ccccc2-c2cc(N(c3ccc(-c4ccccc4)cc3)c3cccc4c3oc3ccc5sc6c7ccccc7ccc6c5c34)ccc21 ; c1ccc(-c2ccc(N(c3ccc(-c4ccc5ccccc5c4)cc3)c3cccc4c3oc3ccc5sc6c7ccccc7ccc6c5c34)cc2)cc1]

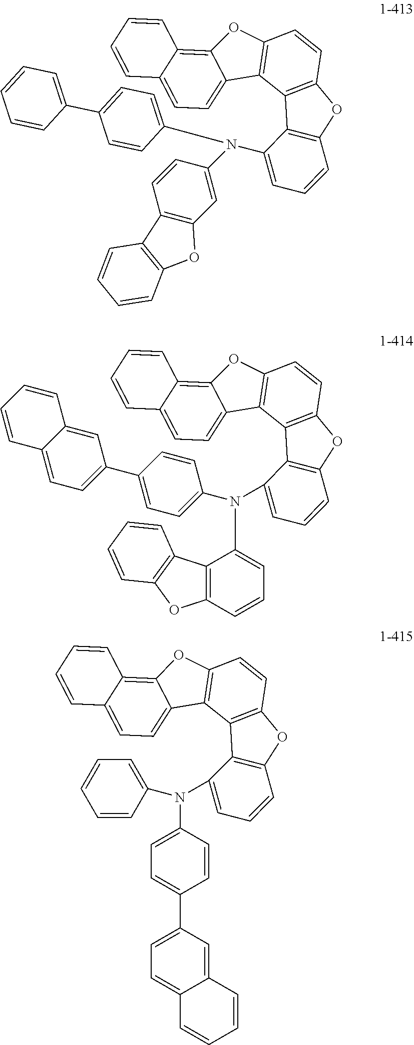

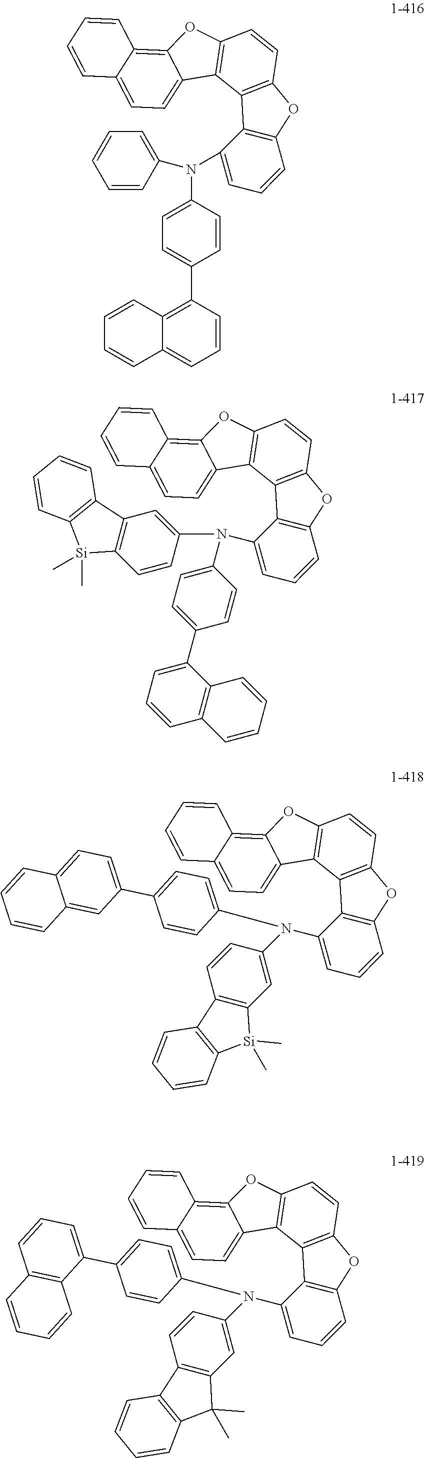

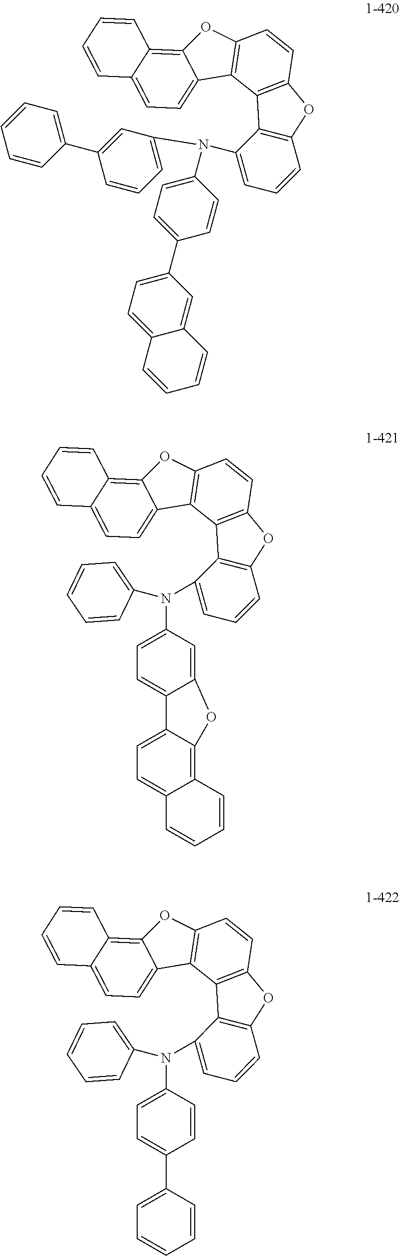 [SMILES: c1ccc(-c2cccc(N(c3ccc(-c4ccc5ccccc5c4)cc3)c3cccc4oc5ccc6oc7c8ccccc8ccc7c6c5c34)c2)cc1 ; c1ccc(N(c2ccc3c(c2)oc2c4ccccc4ccc32)c2cccc3oc4ccc5oc6c7ccccc7ccc6c5c4c23)cc1 ; c1ccc(-c2ccc(N(c3ccccc3)c3cccc4oc5ccc6oc7c8ccccc8ccc7c6c5c34)cc2)cc1]

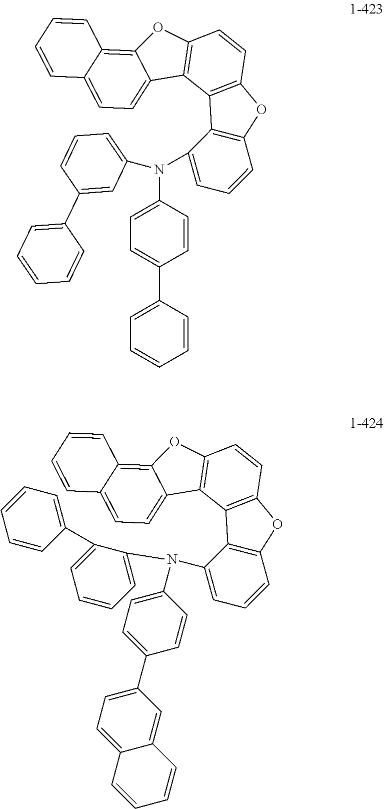 [SMILES: c1ccc(-c2ccc(N(c3cccc(-c4ccccc4)c3)c3cccc4oc5ccc6oc7c8ccccc8ccc7c6c5c34)cc2)cc1 ; c1ccc(-c2ccccc2N(c2ccc(-c3ccc4ccccc4c3)cc2)c2cccc3oc4ccc5oc6c7ccccc7ccc6c5c4c23)cc1]

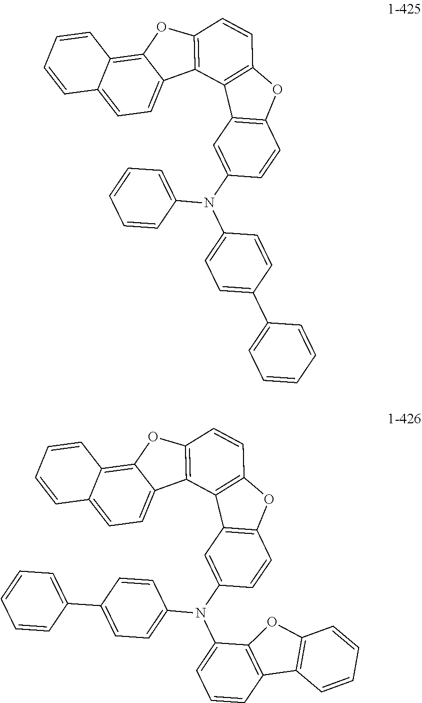 [SMILES: c1ccc(-c2ccc(N(c3ccccc3)c3ccc4oc5ccc6oc7c8ccccc8ccc7c6c5c4c3)cc2)cc1 ; c1ccc(-c2ccc(N(c3ccc4oc5ccc6oc7c8ccccc8ccc7c6c5c4c3)c3cccc4c3oc3ccccc34)cc2)cc1]

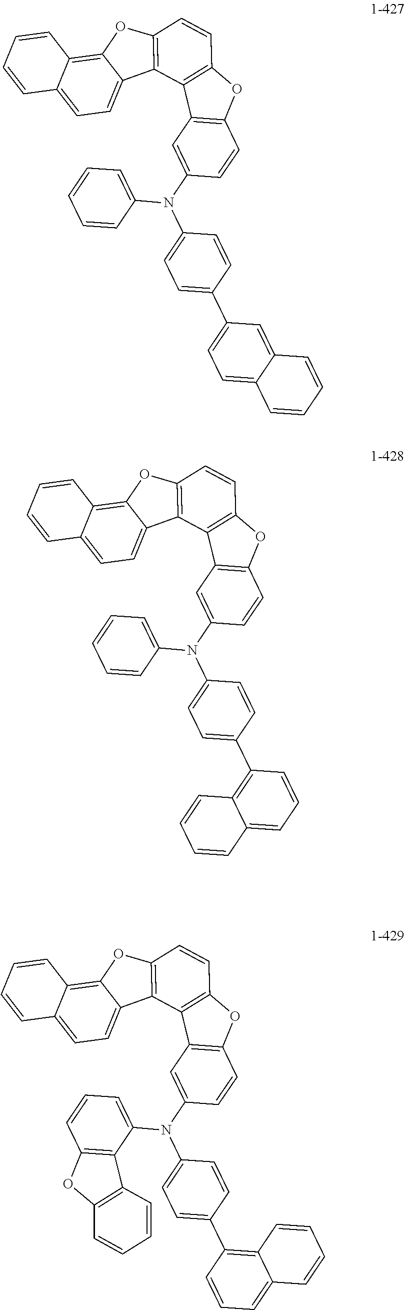 [SMILES: c1ccc(N(c2ccc(-c3ccc4ccccc4c3)cc2)c2ccc3oc4ccc5oc6c7ccccc7ccc6c5c4c3c2)cc1 ; c1ccc(N(c2ccc(-c3cccc4ccccc34)cc2)c2ccc3oc4ccc5oc6c7ccccc7ccc6c5c4c3c2)cc1 ; c1ccc2c(-c3ccc(N(c4ccc5oc6ccc7oc8c9ccccc9ccc8c7c6c5c4)c4cccc5oc6ccccc6c45)cc3)cccc2c1]

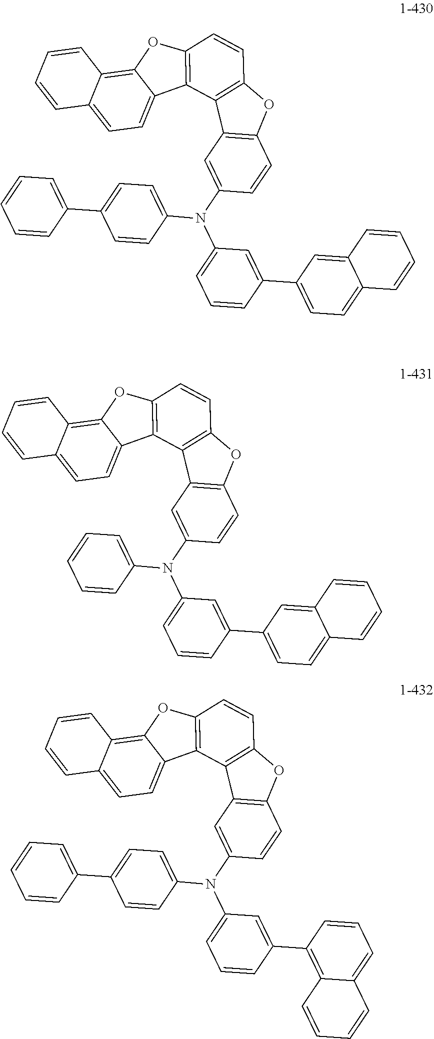

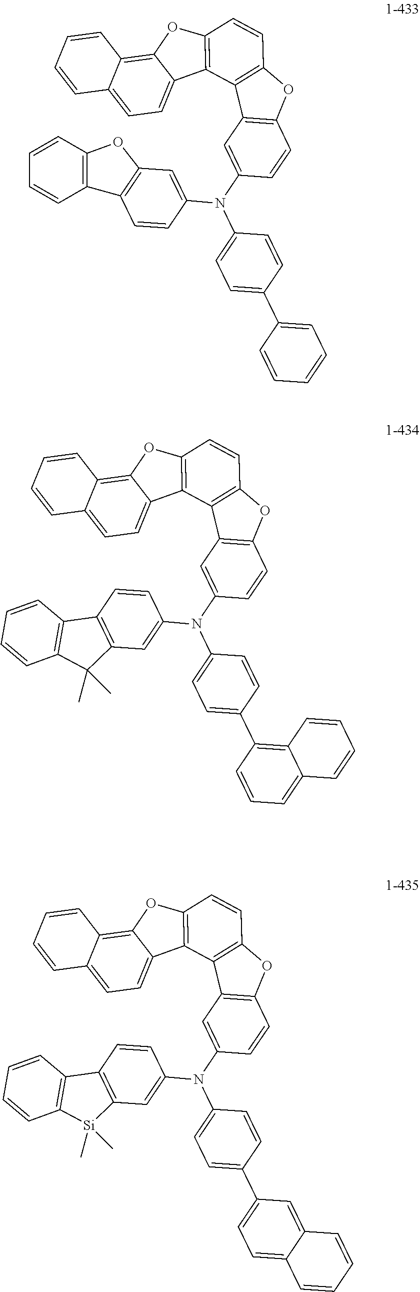 [SMILES: c1ccc(-c2ccc(N(c3ccc4c(c3)oc3ccccc34)c3ccc4oc5ccc6oc7c8ccccc8ccc7c6c5c4c3)cc2)cc1 ; CC1(C)c2ccccc2-c2ccc(N(c3ccc(-c4cccc5ccccc45)cc3)c3ccc4oc5ccc6oc7c8ccccc8ccc7c6c5c4c3)cc21 ; C[Si]1(C)c2ccccc2-c2ccc(N(c3ccc(-c4ccc5ccccc5c4)cc3)c3ccc4oc5ccc6oc7c8ccccc8ccc7c6c5c4c3)cc21]

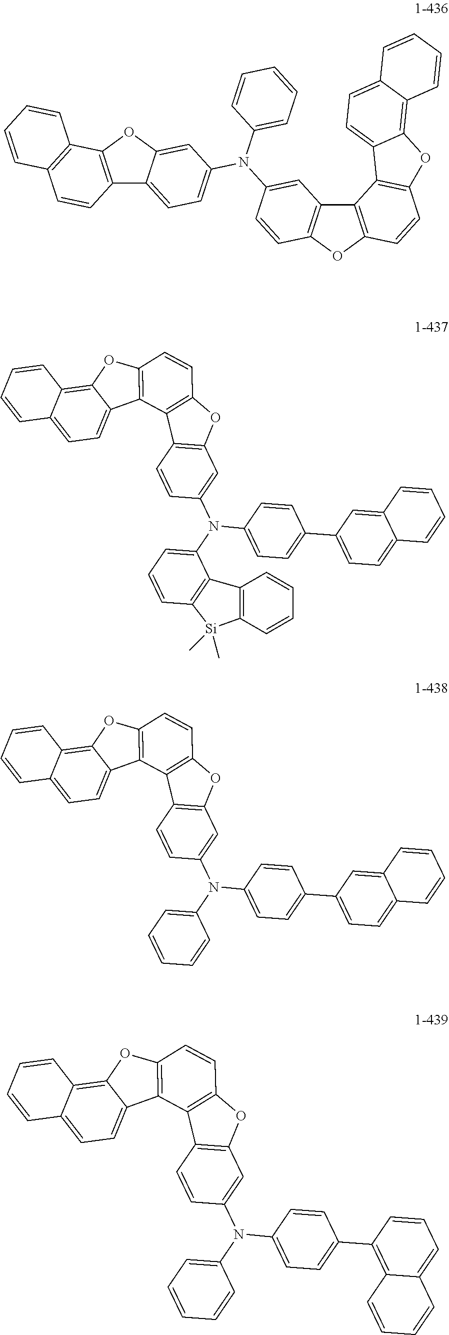 [SMILES: c1ccc(N(c2ccc3c(c2)oc2c4ccccc4ccc32)c2ccc3oc4ccc5oc6c7ccccc7ccc6c5c4c3c2)cc1 ; C[Si]1(C)c2ccccc2-c2c(N(c3ccc(-c4ccc5ccccc5c4)cc3)c3ccc4c(c3)oc3ccc5oc6c7ccccc7ccc6c5c34)cccc21 ; c1ccc(N(c2ccc(-c3ccc4ccccc4c3)cc2)c2ccc3c(c2)oc2ccc4oc5c6ccccc6ccc5c4c23)cc1 ; c1ccc(N(c2ccc(-c3cccc4ccccc34)cc2)c2ccc3c(c2)oc2ccc4oc5c6ccccc6ccc5c4c23)cc1]

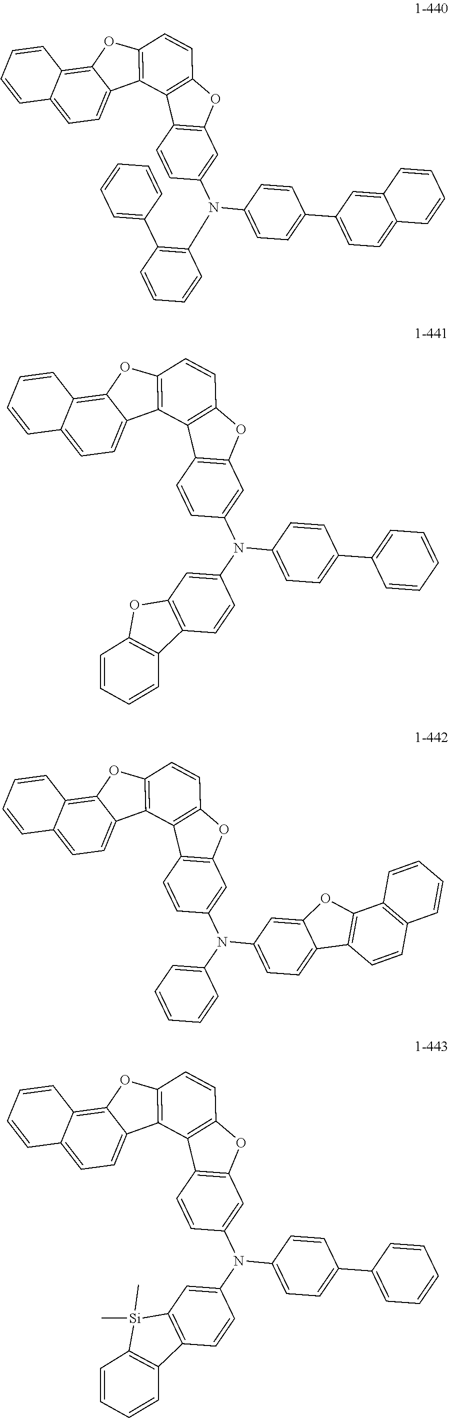 [SMILES: c1ccc(-c2ccccc2N(c2ccc(-c3ccc4ccccc4c3)cc2)c2ccc3c(c2)oc2ccc4oc5c6ccccc6ccc5c4c23)cc1 ; c1ccc(-c2ccc(N(c3ccc4c(c3)oc3ccccc34)c3ccc4c(c3)oc3ccc5oc6c7ccccc7ccc6c5c34)cc2)cc1 ; c1ccc(N(c2ccc3c(c2)oc2c4ccccc4ccc32)c2ccc3c(c2)oc2ccc4oc5c6ccccc6ccc5c4c23)cc1 ; C[Si]1(C)c2ccccc2-c2ccc(N(c3ccc(-c4ccccc4)cc3)c3ccc4c(c3)oc3ccc5oc6c7ccccc7ccc6c5c34)cc21]

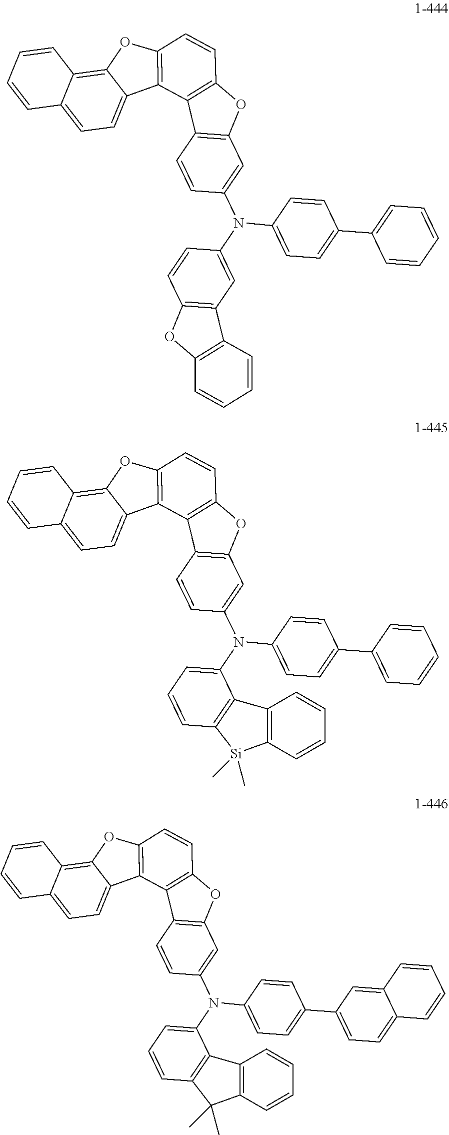 [SMILES: c1ccc(-c2ccc(N(c3ccc4c(c3)oc3ccc5oc6c7ccccc7ccc6c5c34)c3ccc4oc5ccccc5c4c3)cc2)cc1 ; C[Si]1(C)c2ccccc2-c2c(N(c3ccc(-c4ccccc4)cc3)c3ccc4c(c3)oc3ccc5oc6c7ccccc7ccc6c5c34)cccc21 ; CC1(C)c2ccccc2-c2c(N(c3ccc(-c4ccc5ccccc5c4)cc3)c3ccc4c(c3)oc3ccc5oc6c7ccccc7ccc6c5c34)cccc21]

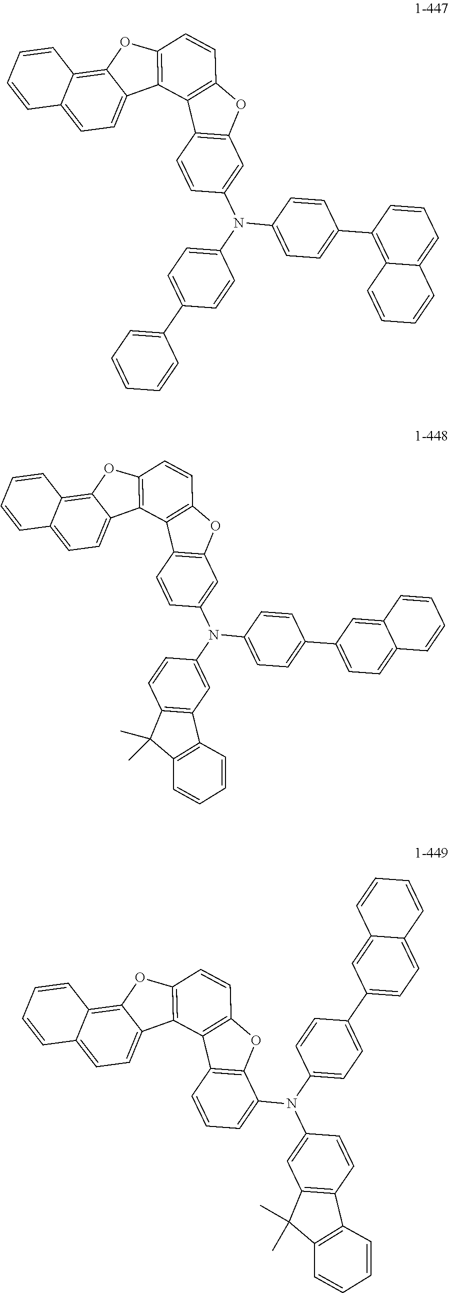 [SMILES: c1ccc(-c2ccc(N(c3ccc(-c4cccc5ccccc45)cc3)c3ccc4c(c3)oc3ccc5oc6c7ccccc7ccc6c5c34)cc2)cc1 ; CC1(C)c2ccccc2-c2cc(N(c3ccc(-c4ccc5ccccc5c4)cc3)c3ccc4c(c3)oc3ccc5oc6c7ccccc7ccc6c5c34)ccc21 ; CC1(C)c2ccccc2-c2ccc(N(c3ccc(-c4ccc5ccccc5c4)cc3)c3cccc4c3oc3ccc5oc6c7ccccc7ccc6c5c34)cc21]

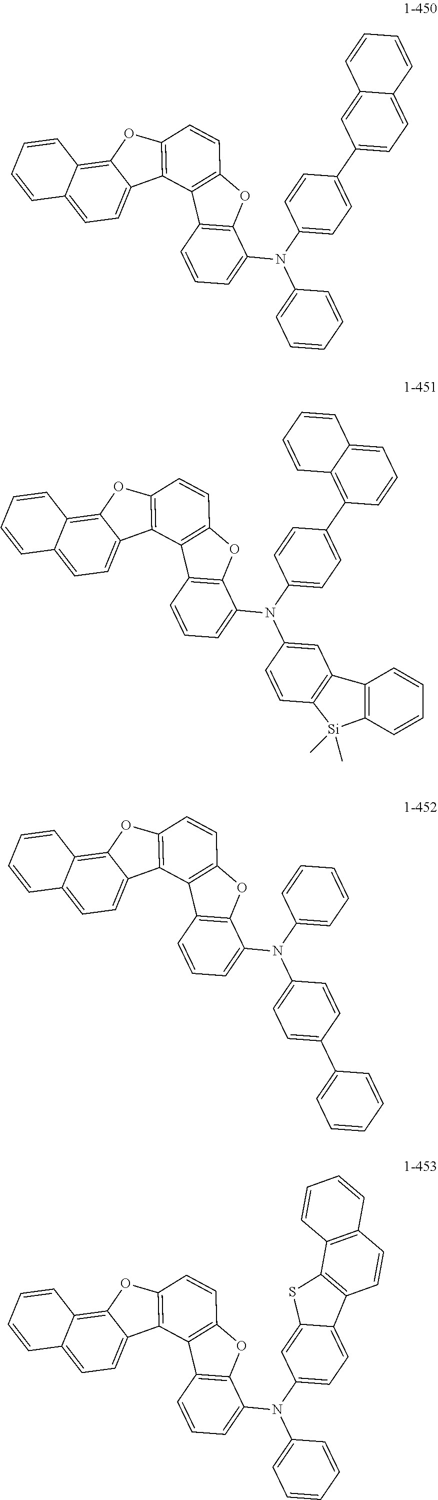 [SMILES: c1ccc(N(c2ccc(-c3ccc4ccccc4c3)cc2)c2cccc3c2oc2ccc4oc5c6ccccc6ccc5c4c23)cc1 ; C[Si]1(C)c2ccccc2-c2cc(N(c3ccc(-c4cccc5ccccc45)cc3)c3cccc4c3oc3ccc5oc6c7ccccc7ccc6c5c34)ccc21 ; c1ccc(-c2ccc(N(c3ccccc3)c3cccc4c3oc3ccc5oc6c7ccccc7ccc6c5c34)cc2)cc1 ; c1ccc(N(c2ccc3c(c2)sc2c4ccccc4ccc32)c2cccc3c2oc2ccc4oc5c6ccccc6ccc5c4c23)cc1]

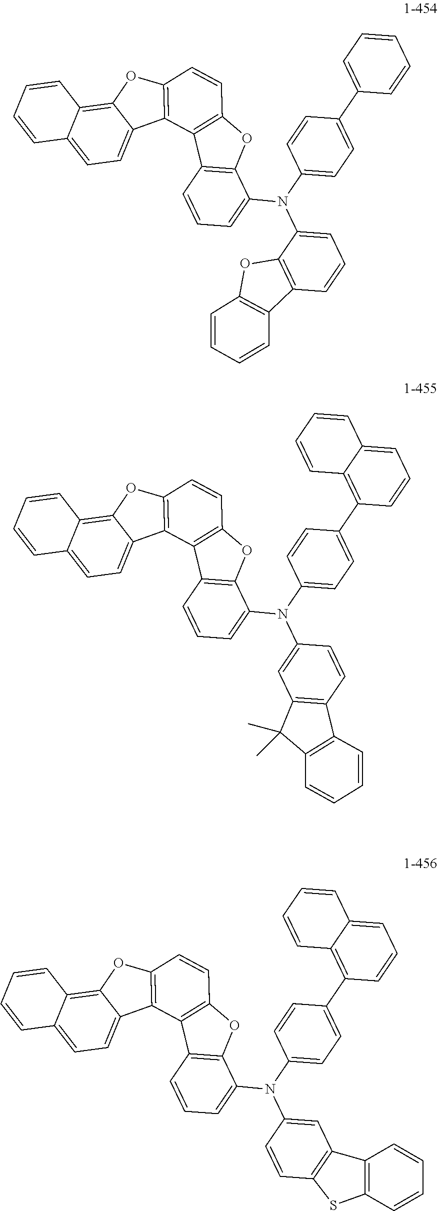 [SMILES: c1ccc(-c2ccc(N(c3cccc4c3oc3ccccc34)c3cccc4c3oc3ccc5oc6c7ccccc7ccc6c5c34)cc2)cc1 ; CC1(C)c2ccccc2-c2ccc(N(c3ccc(-c4cccc5ccccc45)cc3)c3cccc4c3oc3ccc5oc6c7ccccc7ccc6c5c34)cc21 ; c1ccc2c(-c3ccc(N(c4ccc5sc6ccccc6c5c4)c4cccc5c4oc4ccc6oc7c8ccccc8ccc7c6c45)cc3)cccc2c1]

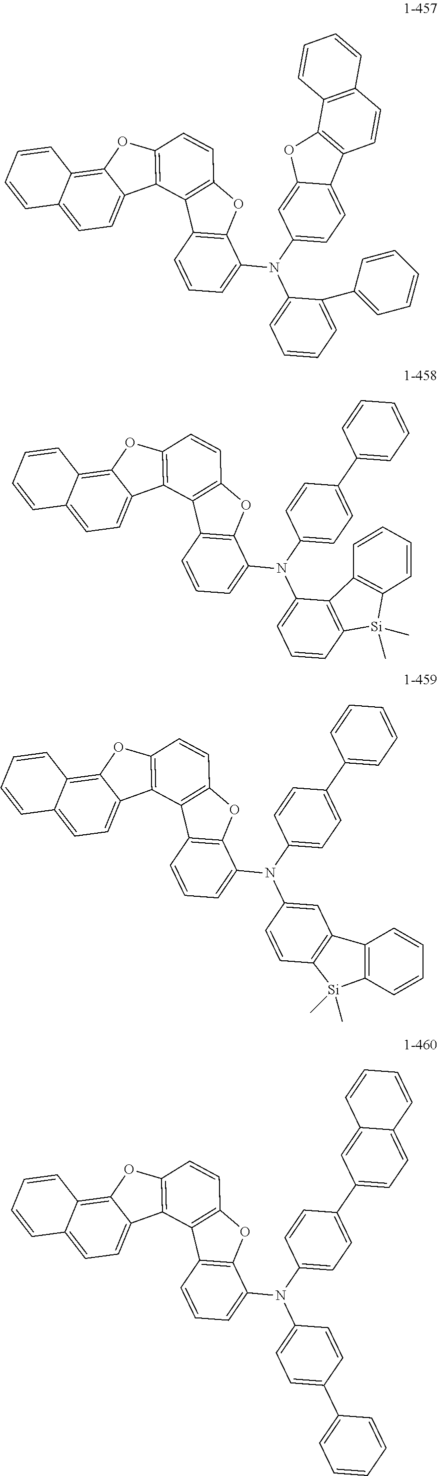 [SMILES: c1ccc(-c2ccccc2N(c2ccc3c(c2)oc2c4ccccc4ccc32)c2cccc3c2oc2ccc4oc5c6ccccc6ccc5c4c23)cc1 ; C[Si]1(C)c2ccccc2-c2c(N(c3ccc(-c4ccccc4)cc3)c3cccc4c3oc3ccc5oc6c7ccccc7ccc6c5c34)cccc21 ; C[Si]1(C)c2ccccc2-c2cc(N(c3ccc(-c4ccccc4)cc3)c3cccc4c3oc3ccc5oc6c7ccccc7ccc6c5c34)ccc21 ; c1ccc(-c2ccc(N(c3ccc(-c4ccc5ccccc5c4)cc3)c3cccc4c3oc3ccc5oc6c7ccccc7ccc6c5c34)cc2)cc1]

A composition for an organic optoelectronic device according to another embodiment includes a first compound and a second compound, wherein the first compound may be the compound for an organic optoelectronic device described above, and the second compound may be a compound for an organic optoelectronic device represented by Chemical Formula 2.

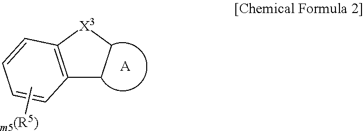

In Chemical Formula 2,
X³ is O, S, N—L^{a}-R^{a}, CR^{b}R^{c}, or SiR^{d}R^{e},
L^{a} is a single bond, or a substituted or unsubstituted C6 to C12 arylene group,
R^{a}, R^{b}, R^{c}, R^{d}, R^{e}, and R⁵ are each independently hydrogen, deuterium, a substituted or unsubstituted C6 to C30 aryl group or a substituted or unsubstituted C2 to C30 heterocyclic group,
m5 is one of integers of 1 to 4, and

A is any one selected from the rings listed in Group II,

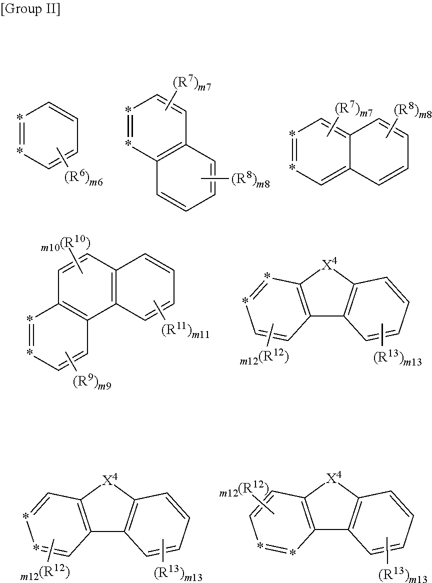

wherein, in Group II,
is a linking point,
X⁴ is O or S,
R⁶ to R¹³ are each independently hydrogen, deuterium, a substituted or unsubstituted C6 to C20 aryl group, or a substituted or unsubstituted C2 to C30 heterocyclic group,
m6, m8, m11, and m13 are each independently one of integers of 1 to 4,
m7, m9, m10, and m12 are each independently an integer of 1 or 2, and at least one of R^{a} and R⁵ to R¹³ is a group represented by Chemical Formula a,

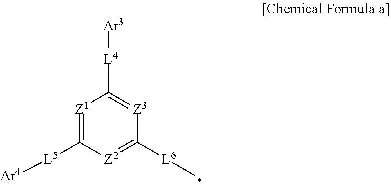

wherein, in Chemical Formula a,
Z¹ to Z³ are each independently N or CR^{f},
at least two of Z¹ to Z³ are N,
R^{f} is hydrogen, deuterium, a substituted or unsubstituted C1 to C30 alkyl group, or a substituted or unsubstituted C6 to C30 aryl group,
L⁴ to L⁶ are each independently a single bond, or a substituted or unsubstituted C6 to C30 arylene group,
Ar³ and Ar⁴ are each independently a substituted or unsubstituted C6 to C30 aryl group, or a substituted or unsubstituted C2 to C30 heteroaryl group, and
is a linking point.

The second compound has a structure substituted with a nitrogen-containing 6-membered ring.

The second compound effectively expands the LUMO energy band by being substituted with a nitrogen-containing 6-membered ring, so when used in the light emitting layer together with the aforementioned first compound, mobility of charges and stability are increased, thereby increasing a balance between holes and electrons to improve luminous efficiency and life-span characteristics of the device and to lower a driving voltage.

In Chemical Formula 2, when m5 is 2 or more, each R⁵ may be the same or different from each other.

In Group II, when m6 is 2 or more, each R⁶ may be the same or different from each other.

In Group II, when m7 is 2 or more, each R⁷ may be the same or different from each other.

In Group II, when m8 is 2 or more, each R⁸ may be the same or different from each other.

In Group II, when m9 is 2 or more, each R⁹ may be the same or different from each other.

In Group II, when m11 is 2 or more, each R¹⁰ may be the same or different from each other.

In Group II, when m12 is 2 or more, each R¹² may be the same or different from each other.

In Group II, when m13 is 2 or more, each R¹³ may be the same or different from each other.

Meanwhile, ring A of the second compound may be selected from the rings listed in Group II. For example, the second compound may be represented by any one of Chemical Formula 2-I to Chemical Formula 2-X.

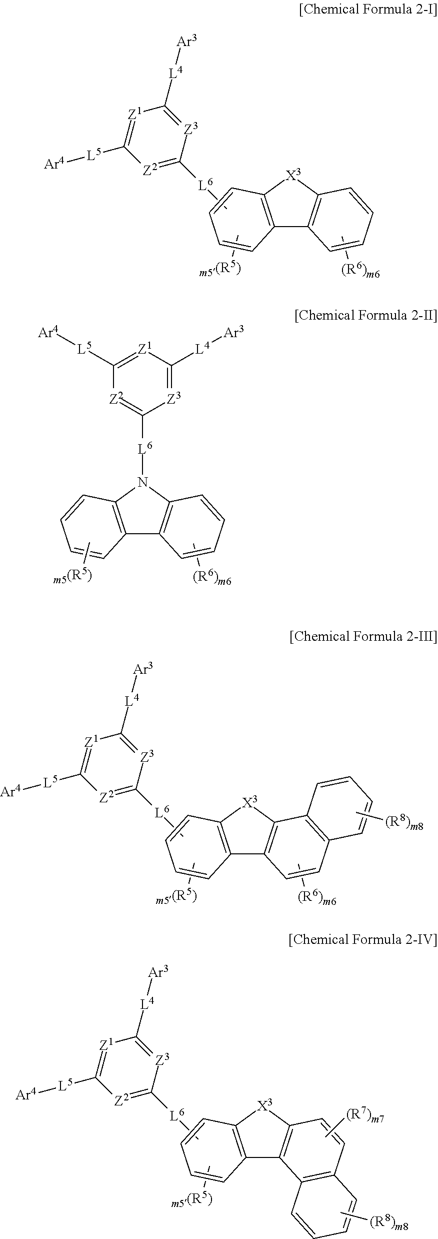

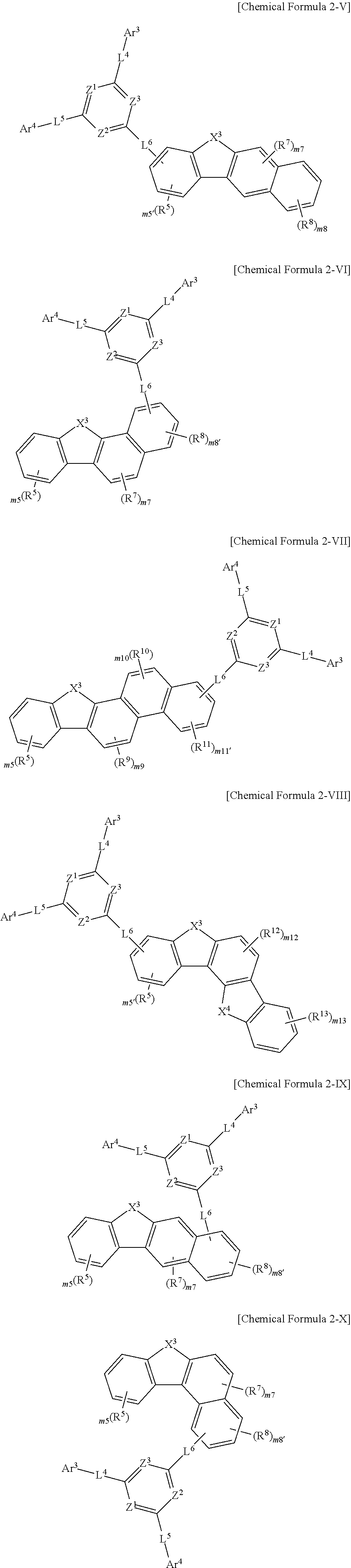

In Chemical Formula 2-I to Chemical Formula 2-X,
X³, X⁴, Z¹ to Z³, R⁵ to R¹³, m5 to m13, L⁴ to L⁶, Ar³, and Ar³ are the same as described above, and
m5′, m8′, and m11′ are each independently one of integers of 1 to 3.

The second compound according to an embodiment may be represented by any one of Chemical Formula 2-I, Chemical Formula 2-III and Chemical Formula 2-VI.

The second compound according to a specific embodiment may be represented by any one Chemical Formula 2-I-3, Chemical Formula 2-III-1, Chemical Formula 2-VI-1, and Chemical Formula 2-VI-3.

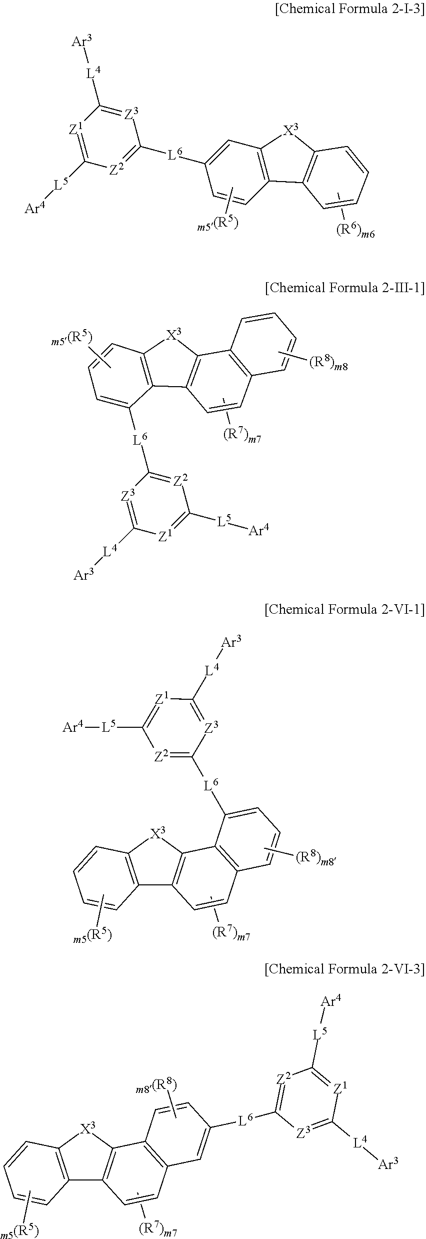

In Chemical Formula 2-I-3, Chemical Formula 2-III-1, Chemical Formula 2-VI-1, and Chemical Formula 2-VI-3,
X², Z¹ to Z³, R⁵ to R⁸, m5′, m6 to m8, m8′, L⁴ to L⁶, Ar³ and Ar⁴ are the same as described above.

For example, Ar³ and Ar⁴ may each independently be a substituted or unsubstituted phenyl group, a substituted or unsubstituted biphenyl group, a substituted or unsubstituted terphenyl group, a substituted or unsubstituted naphthyl group, a substituted or unsubstituted phenanthrenyl group, A substituted or unsubstituted triphenylene group, a substituted or unsubstituted carbazolyl group, a substituted or unsubstituted dibenzofuranyl group, a substituted or unsubstituted dibenzothiophenyl group, or a substituted or unsubstituted dibenzosilolyl group. As a specific example, Ar³ and Ar⁴ may each independently be a substituted or unsubstituted phenyl group, a substituted or unsubstituted biphenyl group, or a substituted or unsubstituted naphthyl group.

For example, L⁴ to L⁶ may each independently be a single bond, a substituted or unsubstituted phenylene group, or a substituted or unsubstituted biphenylene group.

As a specific example, L⁴ and L⁵ may each independently be a single bond or a substituted or unsubstituted phenylene group, and L⁶ may be a single bond.

As an example, R⁵ to R¹³ may each independently be hydrogen, a cyano group, a substituted or unsubstituted C1 to C10 alkyl group, a substituted or unsubstituted C6 to C12 aryl group, or a substituted or unsubstituted C2 to C18 heterocyclic group.

As a specific example, R⁵ to R¹³ may each independently be hydrogen, deuterium, a phenyl group, or a naphthyl group.

For example, X³ may be O, S, CR^{b}R^{c}, or SiR^{d}R^{e}, wherein R^{b}, R^{c}, R^{d}, and R^{e} are each independently a substituted or unsubstituted C1 to C10 alkyl group or a substituted or unsubstituted C6 to C20 aryl group.

As a specific example, R^{b}, R^{c}, R^{d}, and R^{e} may each independently be a methyl group, a substituted or unsubstituted phenyl group, or a substituted or unsubstituted biphenyl group.

For example, the second compound may be one selected from the compounds listed in Group 2.

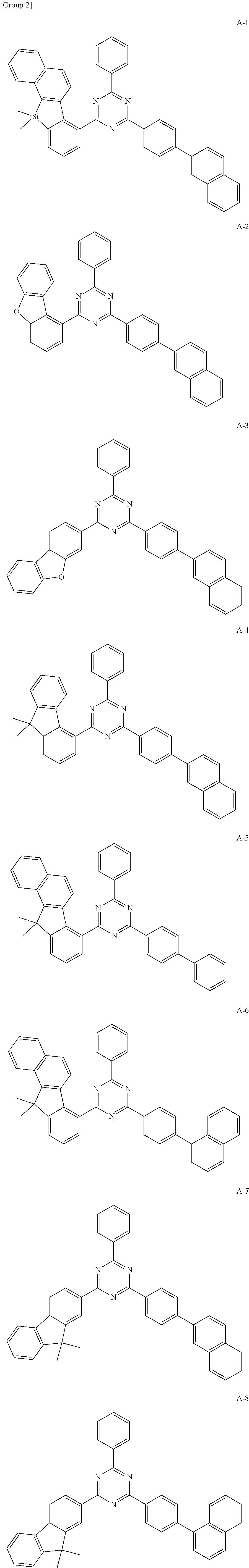

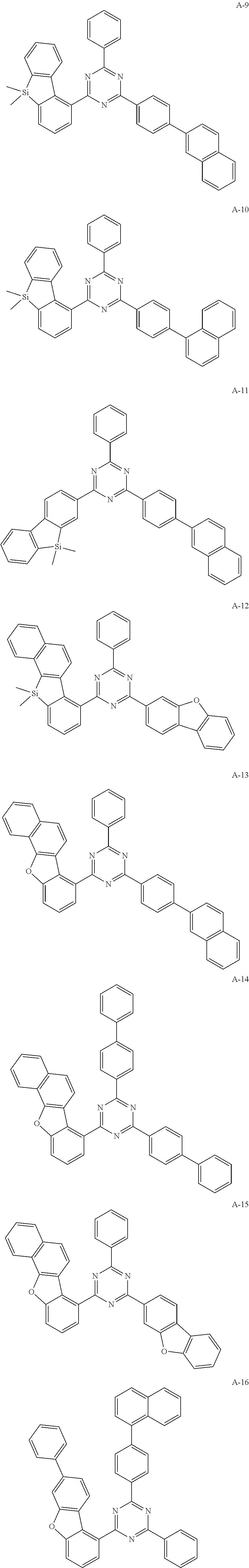

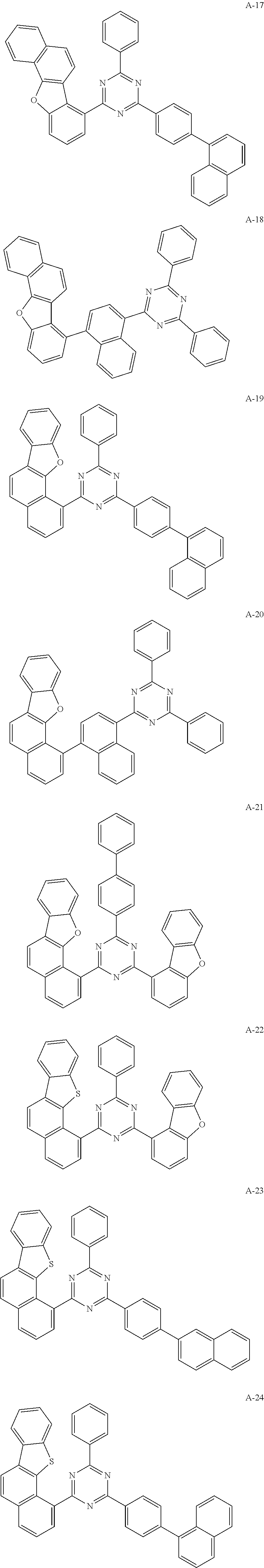

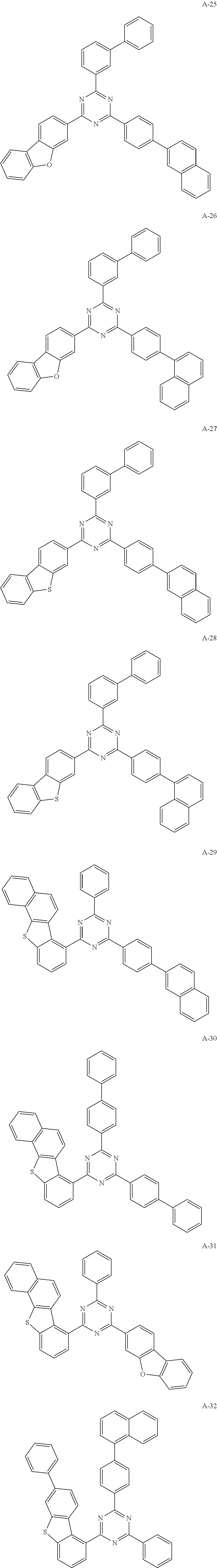

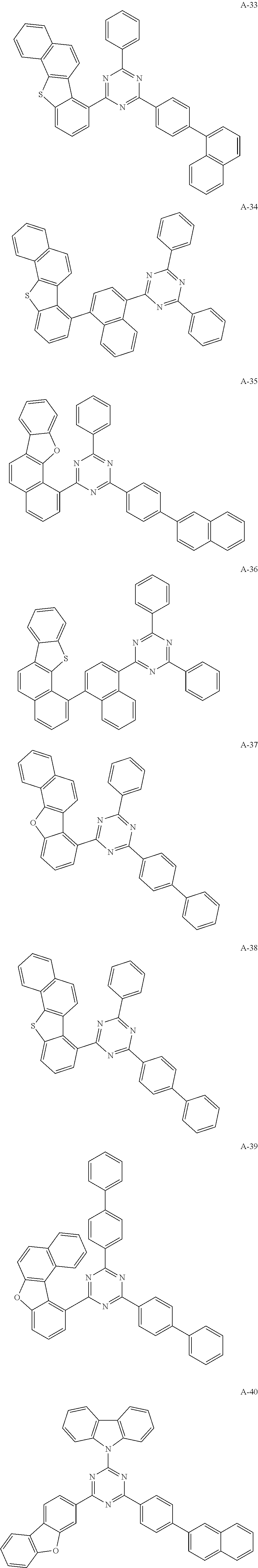

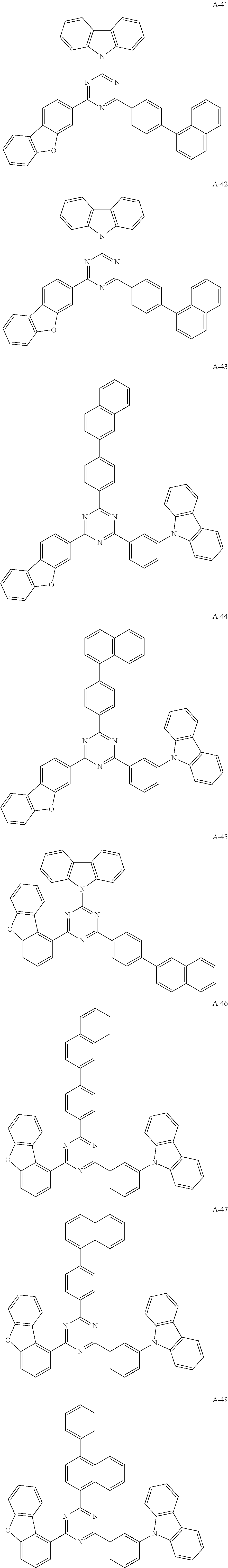

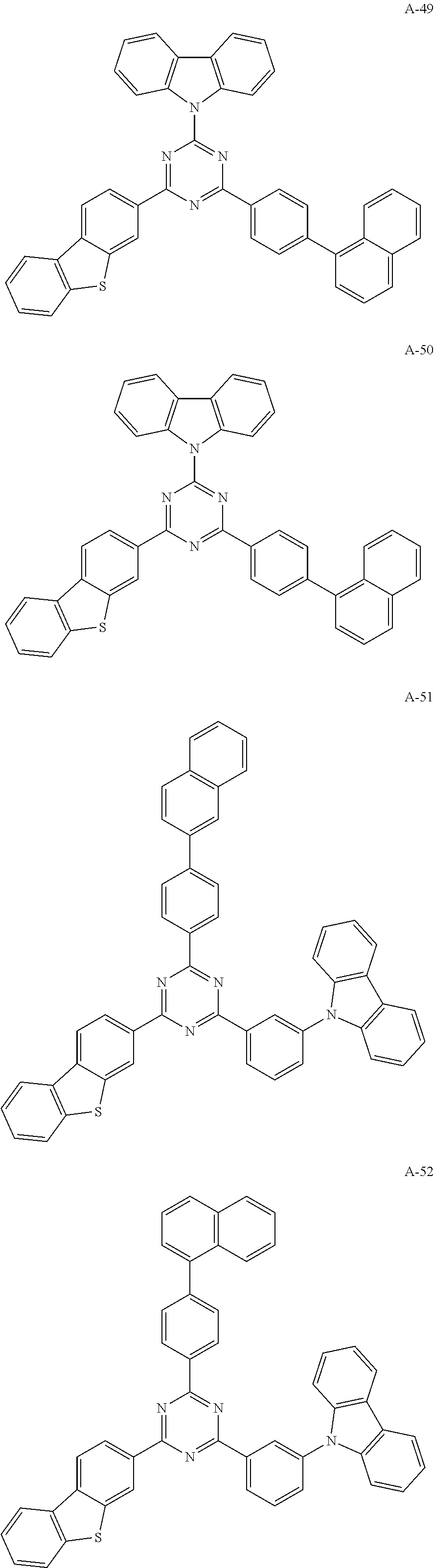 [SMILES: c1ccc2c(-c3ccc(-c4nc(-c5ccc6c(c5)sc5ccccc56)nc(-n5c6ccccc6c6ccccc65)n4)cc3)cccc2c1 ; c1ccc2c(-c3ccc(-c4nc(-c5ccc6c(c5)sc5ccccc56)nc(-n5c6ccccc6c6ccccc65)n4)cc3)cccc2c1 ; c1cc(-c2nc(-c3ccc(-c4ccc5ccccc5c4)cc3)nc(-c3ccc4c(c3)sc3ccccc34)n2)cc(-n2c3ccccc3c3ccccc32)c1 ; c1cc(-c2nc(-c3ccc(-c4cccc5ccccc45)cc3)nc(-c3ccc4c(c3)sc3ccccc34)n2)cc(-n2c3ccccc3c3ccccc32)c1]

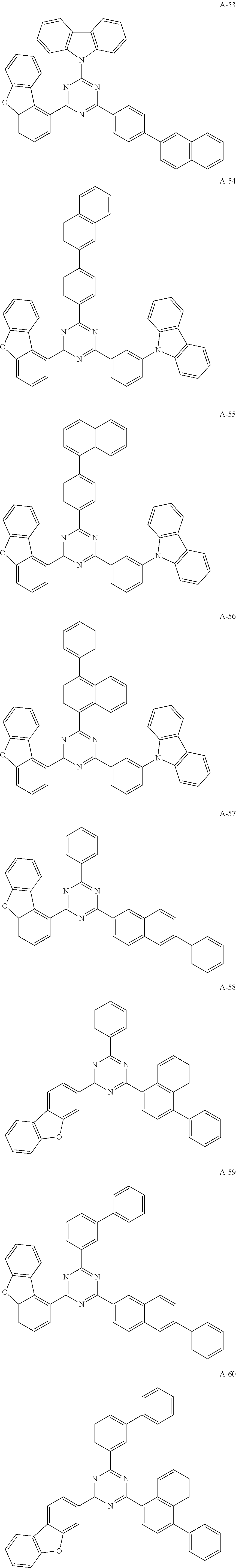

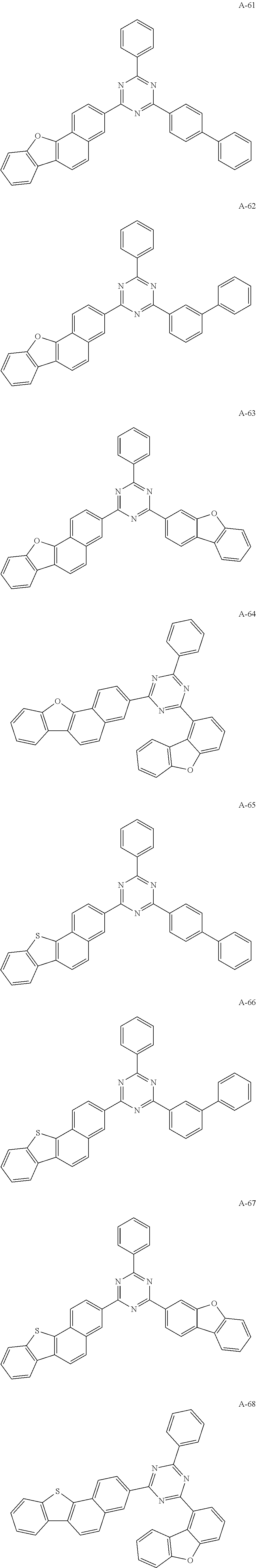

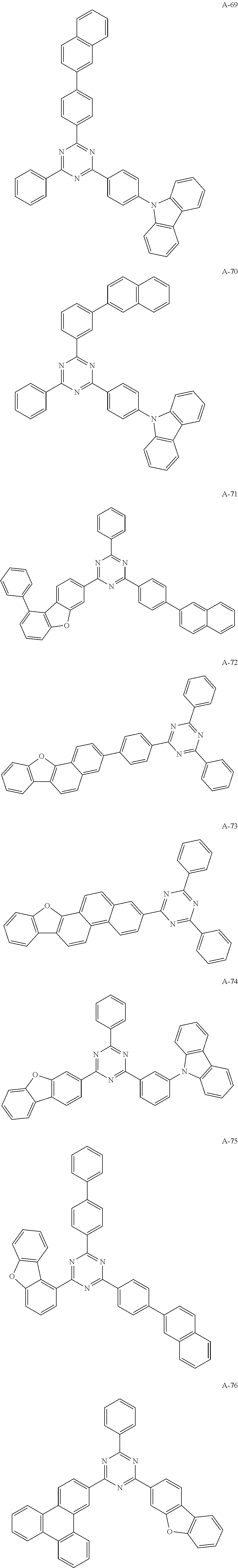

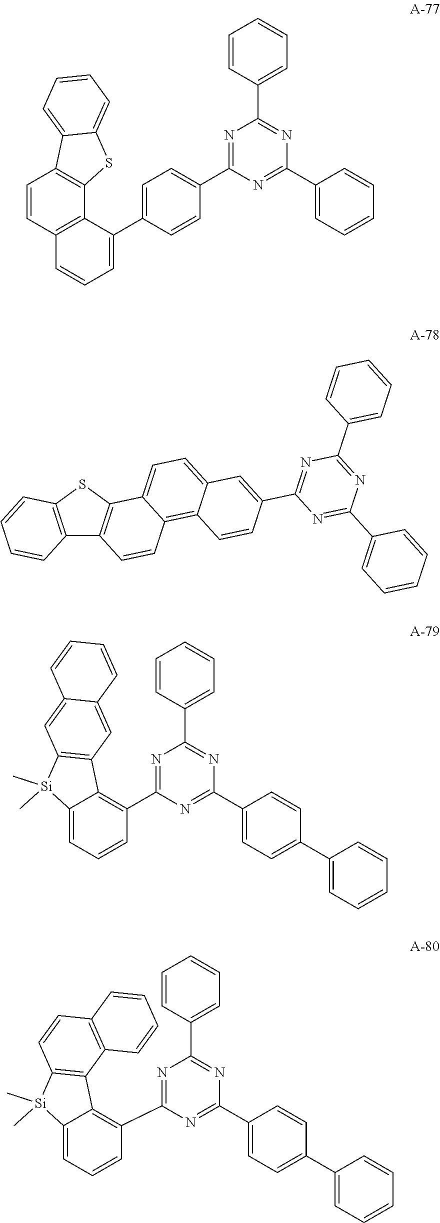 [SMILES: c1ccc(-c2nc(-c3ccccc3)nc(-c3ccc(-c4cccc5ccc6c7ccccc7sc6c45)cc3)n2)cc1 ; c1ccc(-c2nc(-c3ccccc3)nc(-c3ccc4c(ccc5c4ccc4c6ccccc6sc45)c3)n2)cc1 ; C[Si]1(C)c2cc3ccccc3cc2-c2c(-c3nc(-c4ccccc4)nc(-c4ccc(-c5ccccc5)cc4)n3)cccc21 ; C[Si]1(C)c2cccc(-c3nc(-c4ccccc4)nc(-c4ccc(-c5ccccc5)cc4)n3)c2-c2c1ccc1ccccc21]

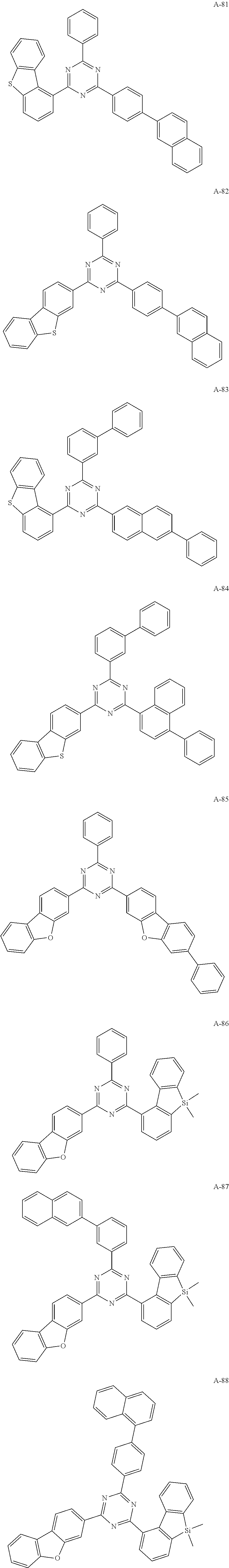

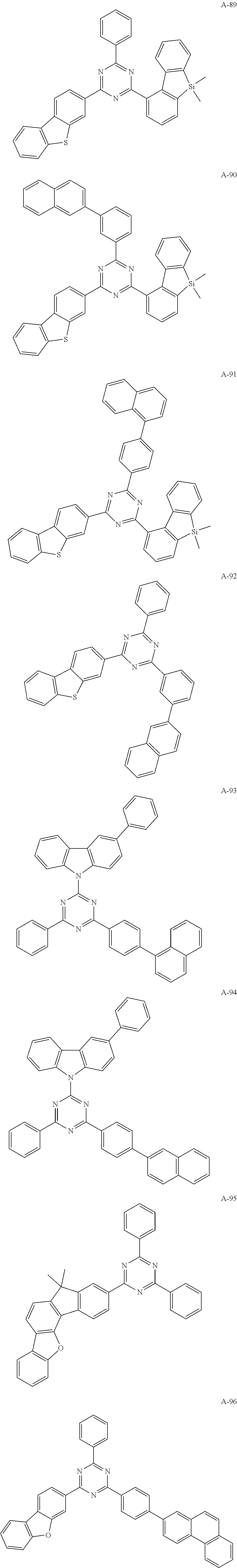

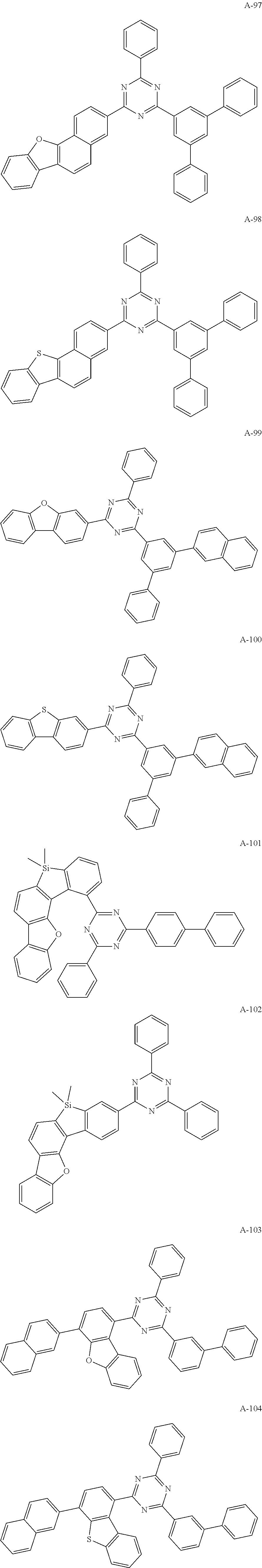

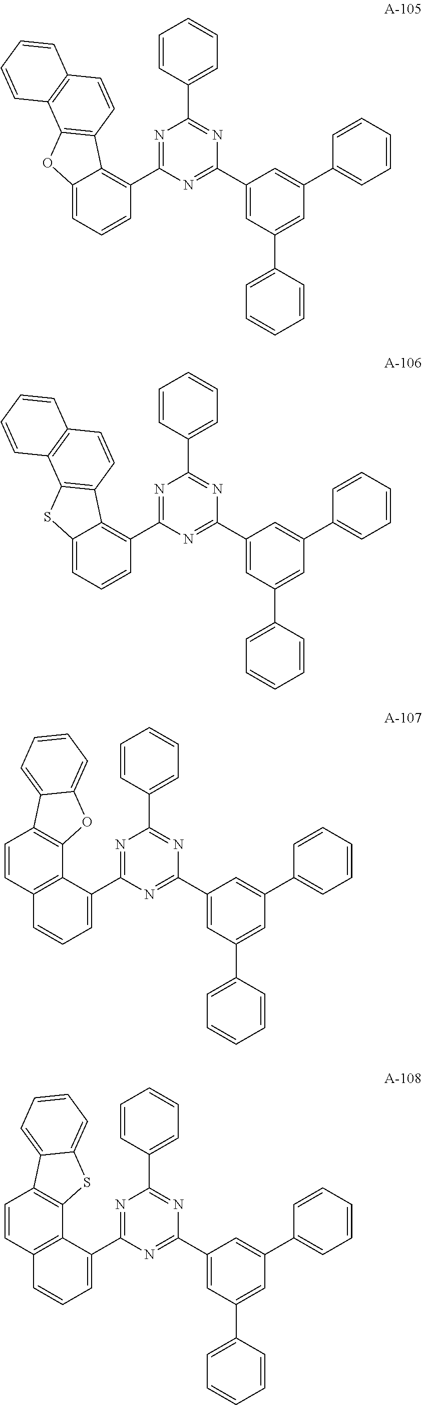 [SMILES: c1ccc(-c2cc(-c3ccccc3)cc(-c3nc(-c4ccccc4)nc(-c4cccc5oc6c7ccccc7ccc6c45)n3)c2)cc1 ; c1ccc(-c2cc(-c3ccccc3)cc(-c3nc(-c4ccccc4)nc(-c4cccc5sc6c7ccccc7ccc6c45)n3)c2)cc1 ; c1ccc(-c2cc(-c3ccccc3)cc(-c3nc(-c4ccccc4)nc(-c4cccc5ccc6c7ccccc7oc6c45)n3)c2)cc1 ; c1ccc(-c2cc(-c3ccccc3)cc(-c3nc(-c4ccccc4)nc(-c4cccc5ccc6c7ccccc7sc6c45)n3)c2)cc1]

A composition for an organic optoelectronic device according to a more specific embodiment of the present invention includes a first compound represented by Chemical Formula 1A-2 or Chemical Formula 1A-3 and a second compound represented by Chemical Formula 2-III-1 or Chemical Formula 2-VI-1.

The first compound and the second compound may be included in a weight ratio of, for example, 1:99 to 99:1. By being included in the above range, efficiency and life-span can be improved by implementing bipolar characteristics by adjusting the appropriate weight ratio using the electron transport capability of the first compound and the hole transport capability of the second compound. Within the above range, they may be included in a weight ratio of, for example, about 10:90 to 90:10, about 20:80 to 80:20, for example, about 20:80 to about 70:30, about 20:80 to about 60:40, and about 30:70 to about 60:40. As a specific example, they may be included in a weight ratio of 40:60, 50:50, or 60:40.

In addition to the first compound and second compound described above, one or more compounds may be further included.

The aforementioned compound for an organic optoelectronic device or a composition for an organic optoelectronic device may be a composition that further includes a dopant.

The dopant may be, for example, a phosphorescent dopant, such as a red, green, or blue phosphorescent dopant, and may be, for example, a red or green phosphorescent dopant.

The dopant is a material mixed with the compound or composition for an organic optoelectronic device in a small amount to cause light emission and may be generally a material such as a metal complex that emits light by multiple excitation into a triplet or more. The dopant may be, for example an inorganic, organic, or organic-inorganic compound, and one or more types thereof may be used.

Examples of the dopant may be a phosphorescent dopant and examples of the phosphorescent dopant may be an organic metal compound including Ir, Pt, Os, Ti, Zr, Hf, Eu, Tb, Tm, Fe, Co, Ni, Ru, Rh, Pd, or a combination thereof. The phosphorescent dopant may be, for example a compound represented by Chemical Formula Z, but is not limited thereto.

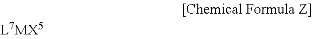

In Chemical Formula Z, M is a metal, and L⁷ and X⁵ are the same or different, and are a ligand to form a complex compound with M.

The M may be for example Ir, Pt, Os, Ti, Zr, Hf, Eu, Tb, Tm, Fe, Co, Ni, Ru, Rh, Pd, or a combination thereof, and L⁷ and X⁵ may be, for example a bidendate ligand.

Examples of the ligands represented by L⁷ and X⁵ may be selected from the Chemical Formulas listed in Group A, but are not limited thereto.

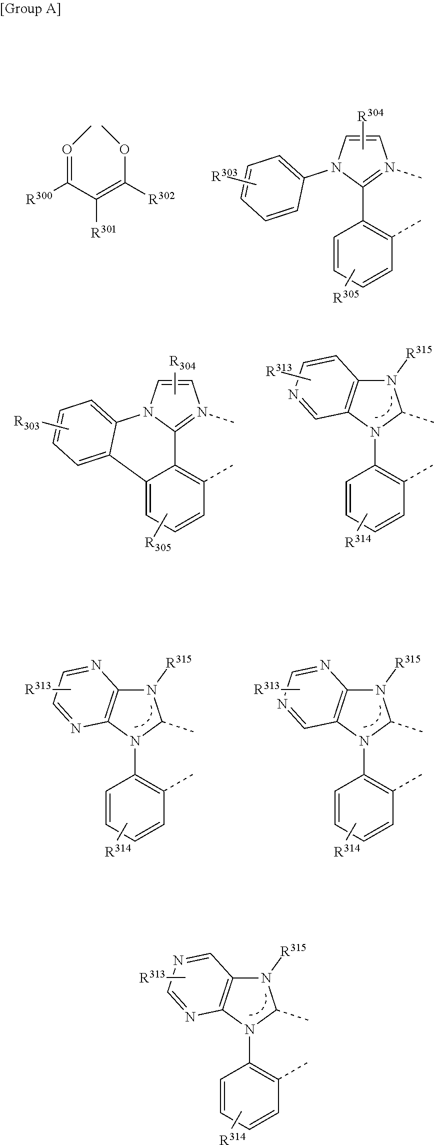

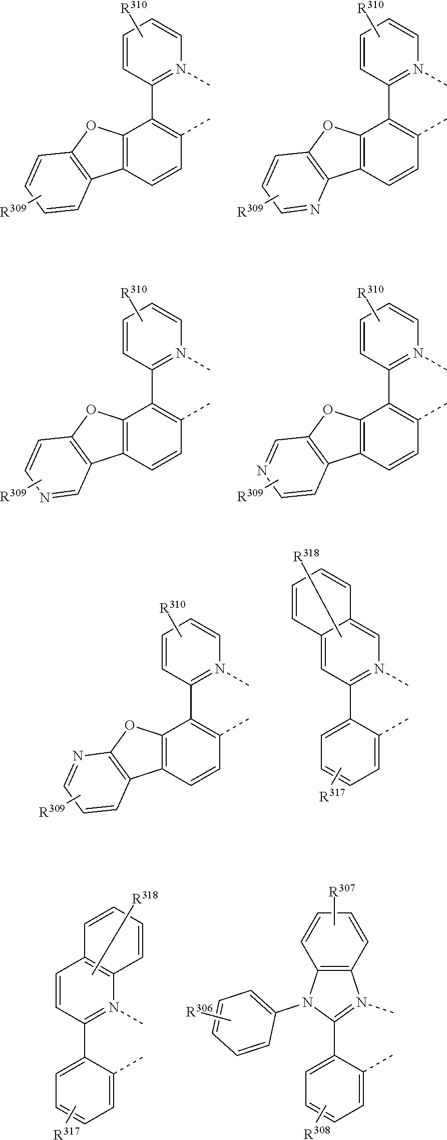

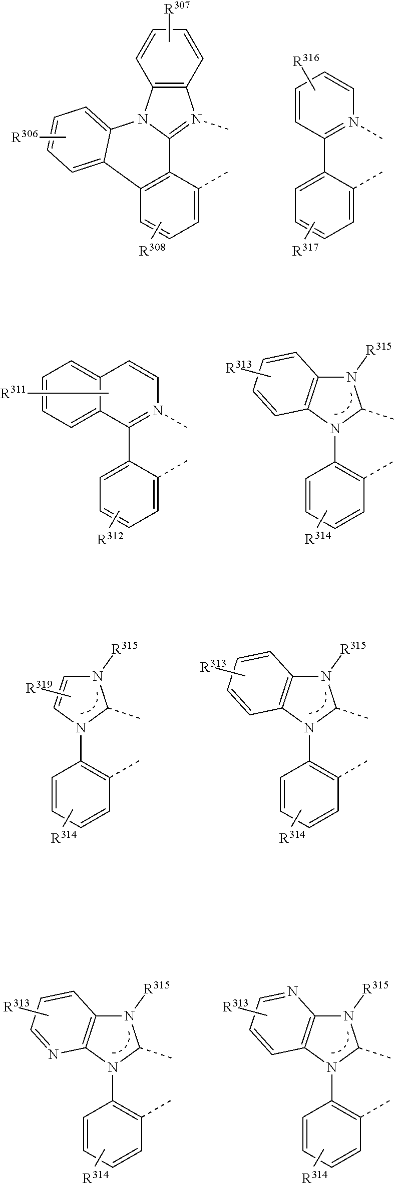

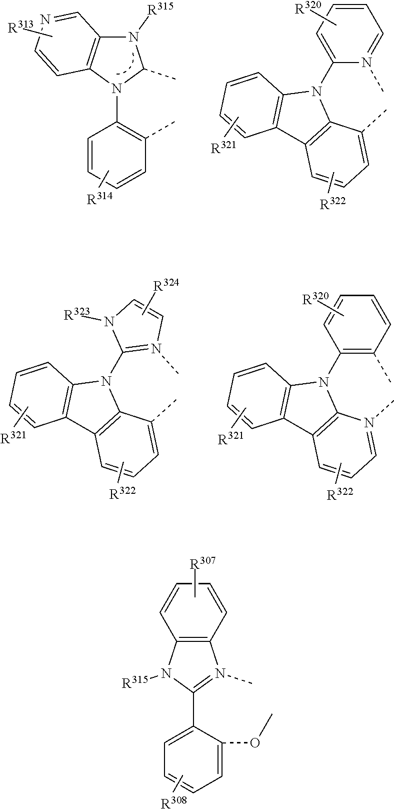

In Group A,
R³⁰⁰ to R³⁰² are each independently hydrogen, deuterium a C1 to C30 alkyl group that is substituted or unsubstituted with a halogen, a C6 to C30 aryl group that is substituted or unsubstituted with a C1 to C30 alkyl, or a halogen, and
R³⁰³ to R³²⁴ are each independently hydrogen, deuterium, a halogen, a substituted or unsubstituted C1 to C30 alkyl group, a substituted or unsubstituted C1 to C30 alkoxy group, a substituted or unsubstituted C3 to C30 cycloalkyl group, a substituted or unsubstituted C2 to C30 alkenyl group, a substituted or unsubstituted C6 to C30 aryl group, a substituted or unsubstituted C1 to C30 heteroaryl group, a substituted or unsubstituted C1 to C30 amino group, a substituted or unsubstituted C6 to C30 arylamino group, SFs, a trialkylsilyl group having a substituted or unsubstituted C1 to C30 alkyl group, a dialkylarylsilyl group having a substituted or unsubstituted C1 to C30 alkyl group and C6 to C30 aryl group, or a triarylsilyl group having a substituted or unsubstituted C6 to C30 aryl group.

For example, a dopant represented by Chemical Formula III may be included.

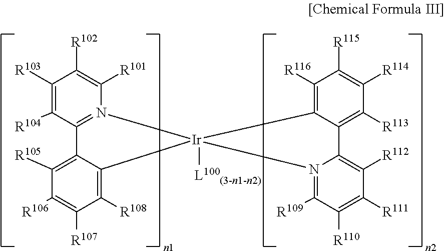

In Chemical Formula III,
R¹⁰¹ to R¹¹⁶ are each independently hydrogen, deuterium, a substituted or unsubstituted C1 to C10 alkyl group, a substituted or unsubstituted C6 to C20 aryl group, or —SiR¹³²R¹³³R¹³⁴,
R¹³² to R¹³⁴ are each independently a C1 to C6 alkyl group,
at least one of R¹⁰¹ to R¹¹⁶ is a functional group represented by Chemical Formula IV-1,
L¹⁰⁰ may be a bidentate ligand of a monovalent anion, and is a ligand that coordinates to iridium through a lone pair of carbons or heteroatoms, and
n¹ and n² are each independently any one of integers of 0 to 3, and n1+n2 is any one of integers of 1 to 3,

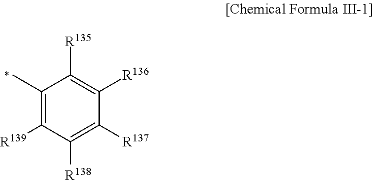

wherein, in Chemical Formula III-1,
R¹³⁵ to R¹³⁹ are each independently hydrogen, deuterium, a substituted or unsubstituted C1 to C10 alkyl group, a substituted or unsubstituted C6 to C20 aryl group, or —SiR¹³²R¹³³R¹³⁴, and
means a part connected to a carbon atom.

For example, the dopant represented by Chemical Formula Z-1 may be included.

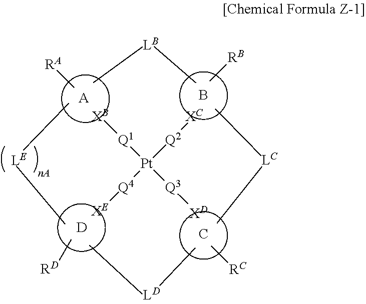

In Chemical Formula Z-1, rings A, B, C, and D are each independently a 5-membered or 6-membered carbocyclic or heterocyclic ring;
R^{A}, R^{B}, R^{C}, and R^{D} are each independently mono-, di-, tri-, or tetra-substitution, or unsubstitution;
L^{B}, L^{C}, and L^{D} are each independently a direct bond, BR, NR, PR, O, S, Se, C═O, S═O, SO₂, CRR′, SiRR′, GeRR′, or a combination thereof,
when nA is 1, L^{E} may be a direct bond, BR, NR, PR, O, S, Se, C═O, S═O, SO₂, CRR′, SiRR′, GeRR′, or a combination thereof; and when nA is 0, L^{E} does not exist;
R^{A}, R^{B}, R^{C}, R^{D}, R, and R′ are each independently hydrogen, deuterium, a halogen, alkyl group, a cycloalkyl group, a heteroalkyl group, arylalkyl group, an alkoxy group, aryloxy group, an amino group, a silyl group, an alkenyl group, a cycloalkenyl group, a heteroalkenyl group, an alkynyl group, aryl group, a heteroaryl group, an acyl group, a carbonyl group, a carboxylic acid group, an ester group, a nitrile group, an isonitrile group, a sulfanyl group, a sulfinyl group, a sulfonyl group, a phosphino group, or a combination thereof; any adjacent R^{A}, R^{B}, R^{C}, R^{D}, R, and R′ are optionally linked to each other to provide a ring; X^{B}, X^{C}, X^{D}, and X^{E} are each independently selected from carbon and nitrogen; and Q¹, Q², Q³, and Q⁴ each represent oxygen or a direct bond.

The dopant according to an embodiment may be a platinum complex, and may be represented by Chemical Formula IV.

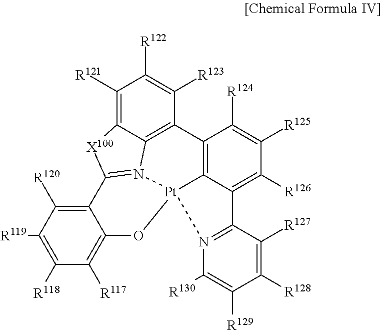

In Chemical Formula IV,
X¹⁰⁰ is selected from O, S, and NR¹³¹,
R¹¹⁷ to R¹³¹ are each independently hydrogen, deuterium, a substituted or unsubstituted C1 to C10 alkyl group, a substituted or unsubstituted C6 to C20 aryl group, or —SiR¹³²R¹³³R¹³⁴,
R¹³² to R¹³⁴ are each independently a C1 to C6 alkyl group, and
at least one of R¹¹⁷ to R¹³¹ may be —SiR¹³²R¹³³R¹³⁴ or a tert-butyl group.

The aforementioned compound for organic optoelectronic devices or composition for organic optoelectronic devices may be formed by a dry film deposition method such as chemical vapor deposition.

Hereinafter, an organic optoelectronic device including the aforementioned compound for an organic optoelectronic device or composition for an organic optoelectronic device will be described.

The organic optoelectronic device may be a suitable device to convert electrical energy into photoenergy and vice versa, e.g., an organic photoelectric device, an organic light emitting diode, an organic solar cell, or an organic photoconductor drum.

Herein, an organic light emitting diode as one example of an organic optoelectronic device is described referring to drawings.

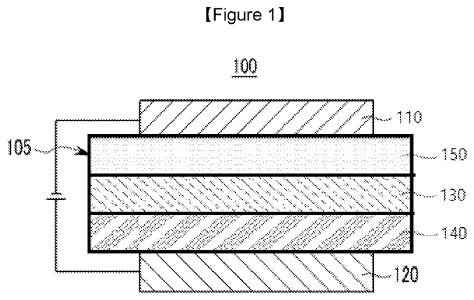

FIG. 1 is a cross-sectional view showing organic light emitting diodes according to embodiments.

Referring to FIG. 1, an organic light emitting diode 100 according to an embodiment includes an anode 120 and a cathode 110 facing each other and an organic layer 105 disposed between the anode 120 and cathode 110.

The anode 120 may be made of a conductor having a large work function to help hole injection, and may be for example a metal, a metal oxide and/or a conductive polymer. The anode 120 may be, for example a metal such as nickel, platinum, vanadium, chromium, copper, zinc, gold, and the like or an alloy thereof; a metal oxide such as zinc oxide, indium oxide, indium tin oxide (ITO), indium zinc oxide (IZO), and the like; a combination of a metal and an oxide such as ZnO and Al or SnO₂ and Sb; a conductive polymer such as poly(3-methylthiophene), poly(3,4-(ethylene-1,2-dioxy)thiophene) (PEDOT), polypyrrole, and polyaniline, but is not limited thereto.

The cathode 110 may be made of a conductor having a small work function to help electron injection, and may be for example a metal, a metal oxide, and/or a conductive polymer. The cathode 110 may be for example a metal such as magnesium, calcium, sodium, potassium, titanium, indium, yttrium, lithium, gadolinium, aluminum silver, tin, lead, cesium, barium, and the like, or an alloy thereof; a multi-layer structure material such as LiF/Al, LiO₂/Al, LiF/Ca, and BaF₂/Ca, but is not limited thereto.

The organic layer 105 may include the aforementioned compound for an organic optoelectronic device or composition for an organic optoelectronic device.

The organic layer 105 may include a light emitting layer 130 and the light emitting layer 130 may include the aforementioned compound for an organic optoelectronic device or composition for an organic optoelectronic device.

The composition for an organic optoelectronic device further including a dopant may be, for example, a green light emitting composition.

The light-emitting layer 130 may include, for example, the aforementioned compound for organic optoelectronic devices or composition for organic optoelectronic devices, respectively, as a phosphorescent host.

The organic layer may further include a charge transport region in addition to the light emitting layer.

The charge transport region may be, for example, the hole transport region 140.

The hole transport region 140 may further increase hole injection and/or hole mobility between the anode 120 and the light emitting layer 130 and block electrons.

Specifically, the hole transport region 140 may include a hole transport layer between the anode 120 and the light emitting layer 130, and a hole transport auxiliary layer between the light emitting layer 130 and the hole transport layer, and at least one of the compounds of Group A may be included in at least one of the hole transport layer and the hole transport auxiliary layer.

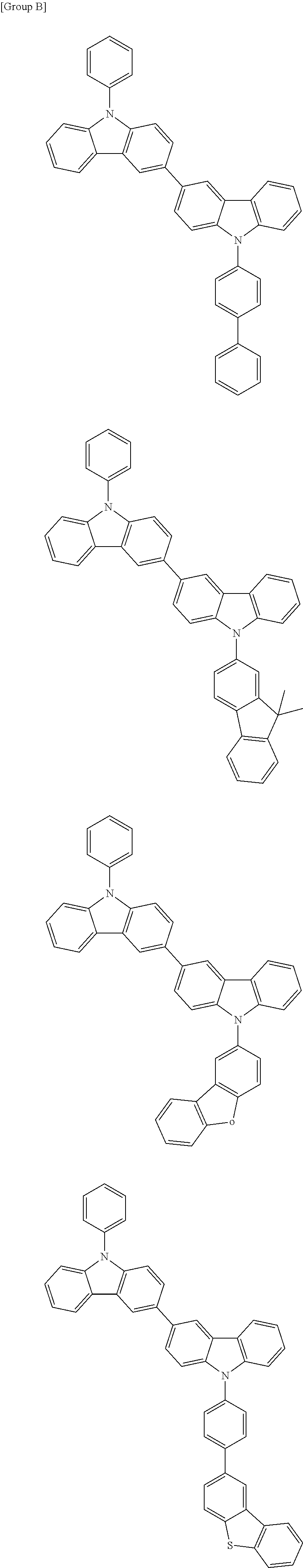

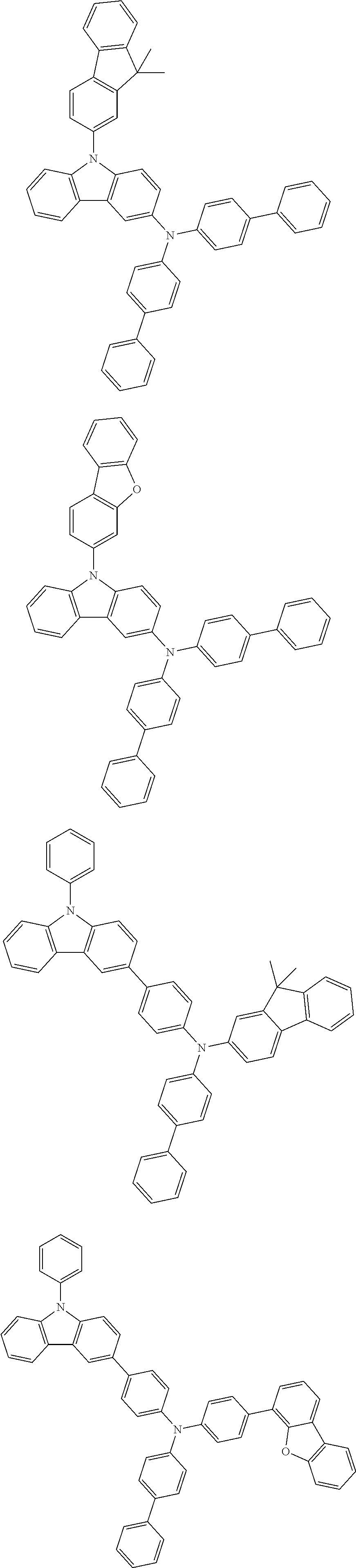

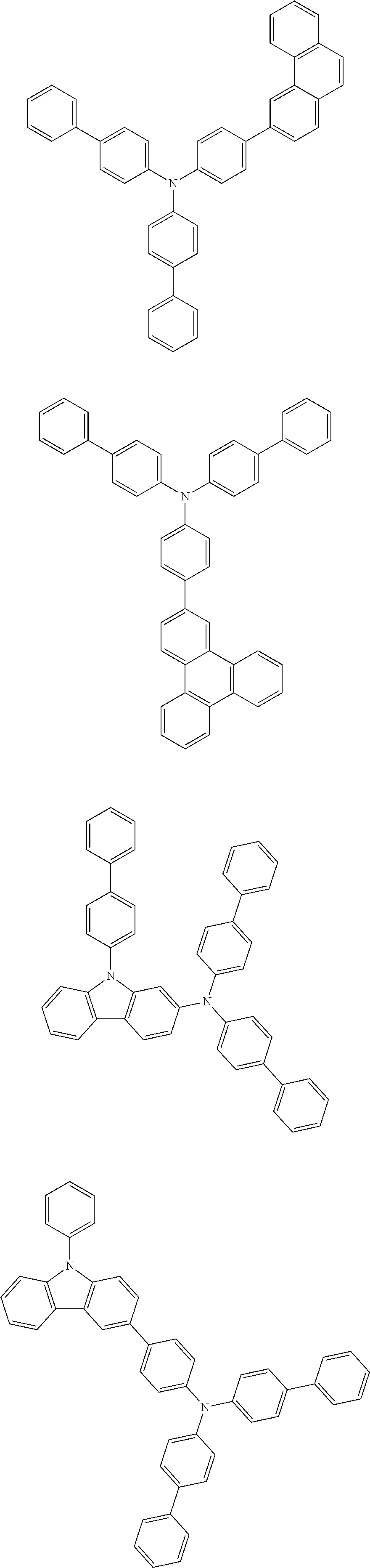 [SMILES: c1ccc(-c2ccc(N(c3ccc(-c4ccccc4)cc3)c3ccc(-c4ccc5ccc6ccccc6c5c4)cc3)cc2)cc1 ; c1ccc(-c2ccc(N(c3ccc(-c4ccccc4)cc3)c3ccc(-c4ccc5c6ccccc6c6ccccc6c5c4)cc3)cc2)cc1 ; c1ccc(-c2ccc(N(c3ccc(-c4ccccc4)cc3)c3ccc4c5ccccc5n(-c5ccc(-c6ccccc6)cc5)c4c3)cc2)cc1 ; c1ccc(-c2ccc(N(c3ccc(-c4ccccc4)cc3)c3ccc(-c4ccc5c(c4)c4ccccc4n5-c4ccccc4)cc3)cc2)cc1]

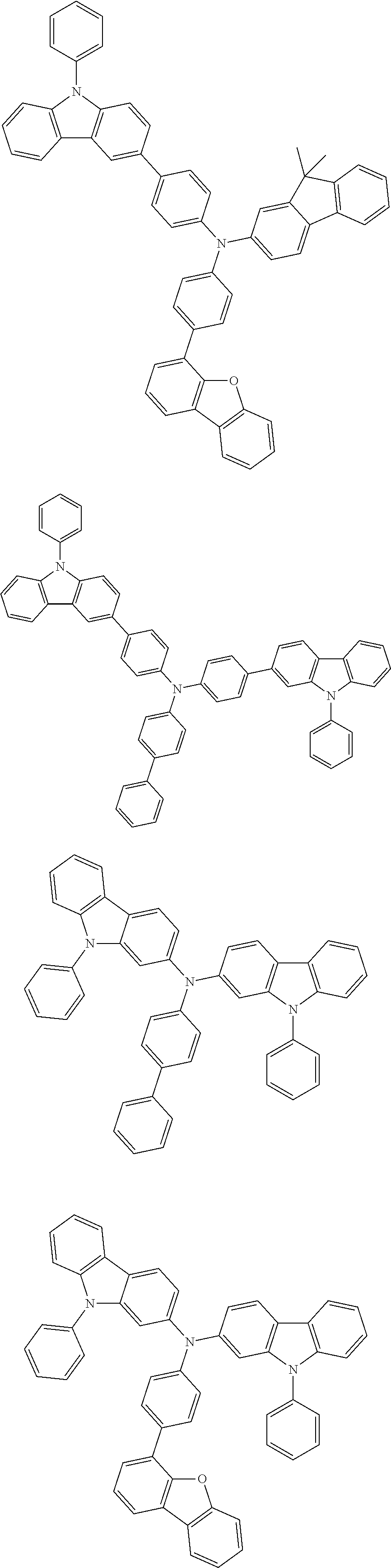 [SMILES: CC1(C)c2ccccc2-c2ccc(N(c3ccc(-c4ccc5c(c4)c4ccccc4n5-c4ccccc4)cc3)c3ccc(-c4cccc5c4oc4ccccc45)cc3)cc21 ; c1ccc(-c2ccc(N(c3ccc(-c4ccc5c(c4)c4ccccc4n5-c4ccccc4)cc3)c3ccc(-c4ccc5c6ccccc6n(-c6ccccc6)c5c4)cc3)cc2)cc1 ; c1ccc(-c2ccc(N(c3ccc4c5ccccc5n(-c5ccccc5)c4c3)c3ccc4c5ccccc5n(-c5ccccc5)c4c3)cc2)cc1 ; c1ccc(-n2c3ccccc3c3ccc(N(c4ccc(-c5cccc6c5oc5ccccc56)cc4)c4ccc5c6ccccc6n(-c6ccccc6)c5c4)cc32)cc1]

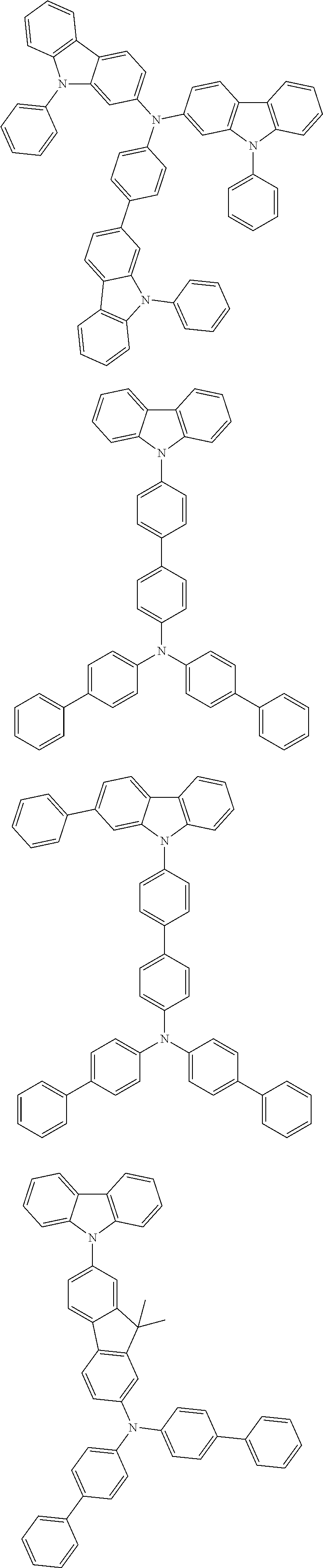

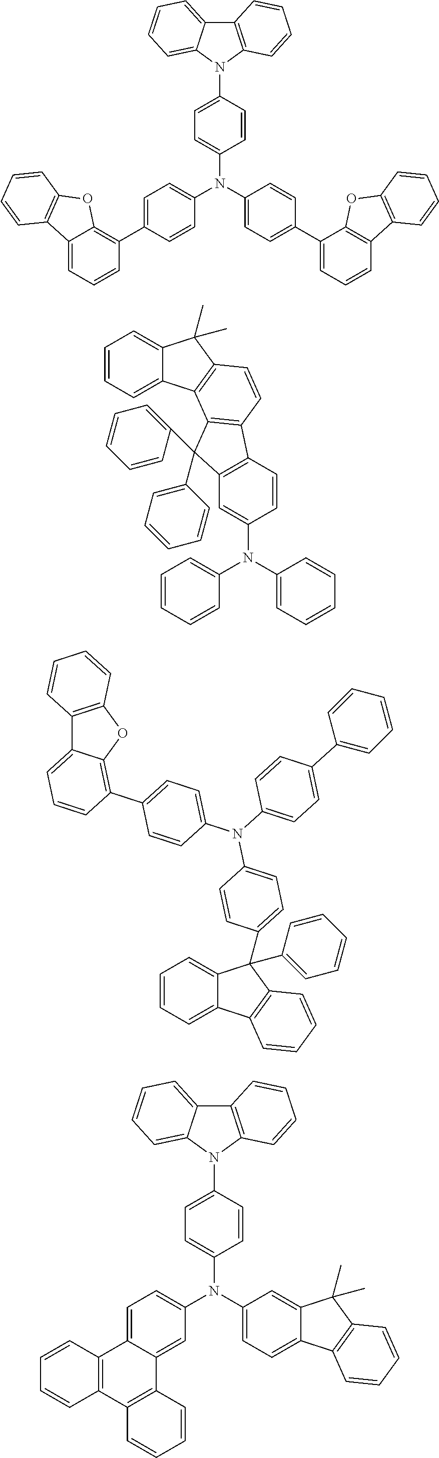

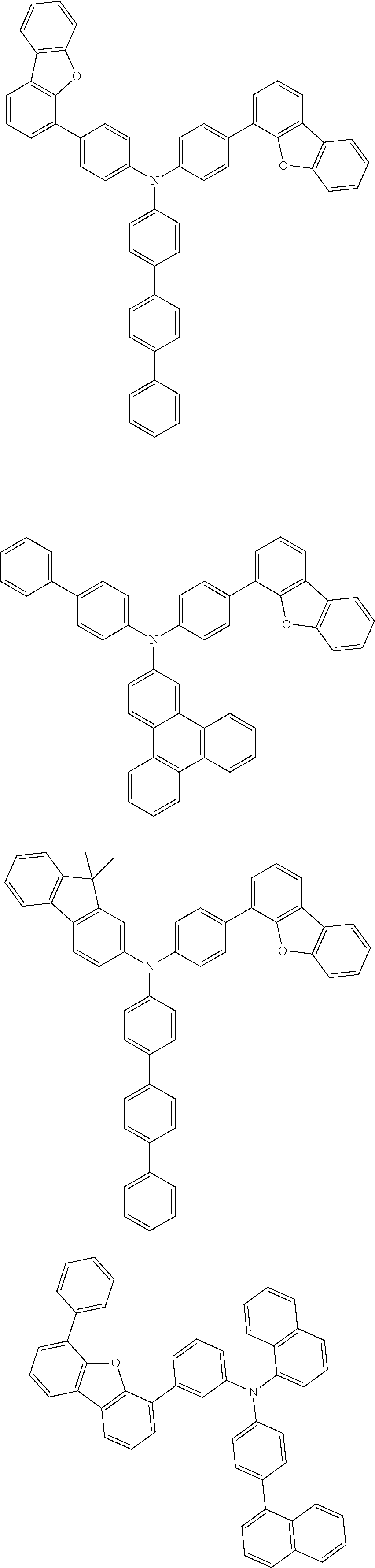 [SMILES: c1ccc(-c2ccc(-c3ccc(N(c4ccc(-c5cccc6c5oc5ccccc56)cc4)c4ccc(-c5cccc6c5oc5ccccc56)cc4)cc3)cc2)cc1 ; c1ccc(-c2ccc(N(c3ccc(-c4cccc5c4oc4ccccc45)cc3)c3ccc4c5ccccc5c5ccccc5c4c3)cc2)cc1 ; CC1(C)c2ccccc2-c2ccc(N(c3ccc(-c4ccc(-c5ccccc5)cc4)cc3)c3ccc(-c4cccc5c4oc4ccccc45)cc3)cc21 ; c1ccc(-c2cccc3c2oc2c(-c4cccc(N(c5ccc(-c6cccc7ccccc67)cc5)c5cccc6ccccc56)c4)cccc23)cc1]

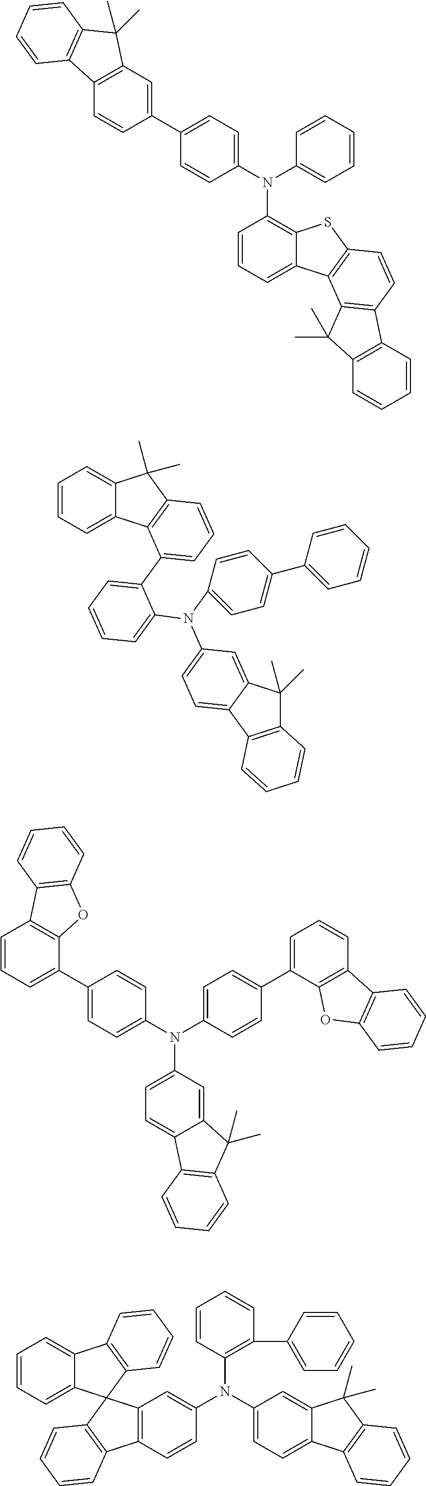 [SMILES: CC1(C)c2ccccc2-c2ccc(-c3ccc(N(c4ccccc4)c4cccc5c4sc4ccc6c(c45)C(C)(C)c4ccccc4-6)cc3)cc21 ; CC1(C)c2ccccc2-c2ccc(N(c3ccc(-c4ccccc4)cc3)c3ccccc3-c3cccc4c3-c3ccccc3C4(C)C)cc21 ; CC1(C)c2ccccc2-c2ccc(N(c3ccc(-c4cccc5c4oc4ccccc45)cc3)c3ccc(-c4cccc5c4oc4ccccc45)cc3)cc21 ; CC1(C)c2ccccc2-c2ccc(N(c3ccc4c(c3)C3(c5ccccc5-c5ccccc53)c3ccccc3-4)c3ccccc3-c3ccccc3)cc21]

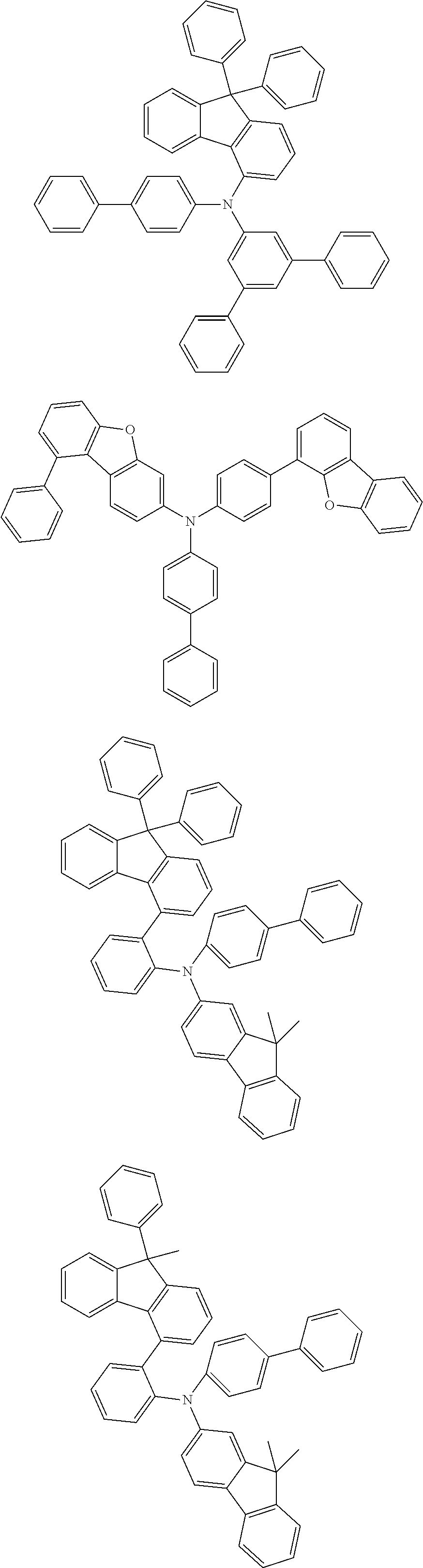 [SMILES: c1ccc(-c2ccc(N(c3cc(-c4ccccc4)cc(-c4ccccc4)c3)c3cccc4c3-c3ccccc3C4(c3ccccc3)c3ccccc3)cc2)cc1 ; c1ccc(-c2ccc(N(c3ccc(-c4cccc5c4oc4ccccc45)cc3)c3ccc4c(c3)oc3cccc(-c5ccccc5)c34)cc2)cc1 ; CC1(C)c2ccccc2-c2ccc(N(c3ccc(-c4ccccc4)cc3)c3ccccc3-c3cccc4c3-c3ccccc3C4(c3ccccc3)c3ccccc3)cc21 ; CC1(C)c2ccccc2-c2ccc(N(c3ccc(-c4ccccc4)cc3)c3ccccc3-c3cccc4c3-c3ccccc3C4(C)c3ccccc3)cc21]

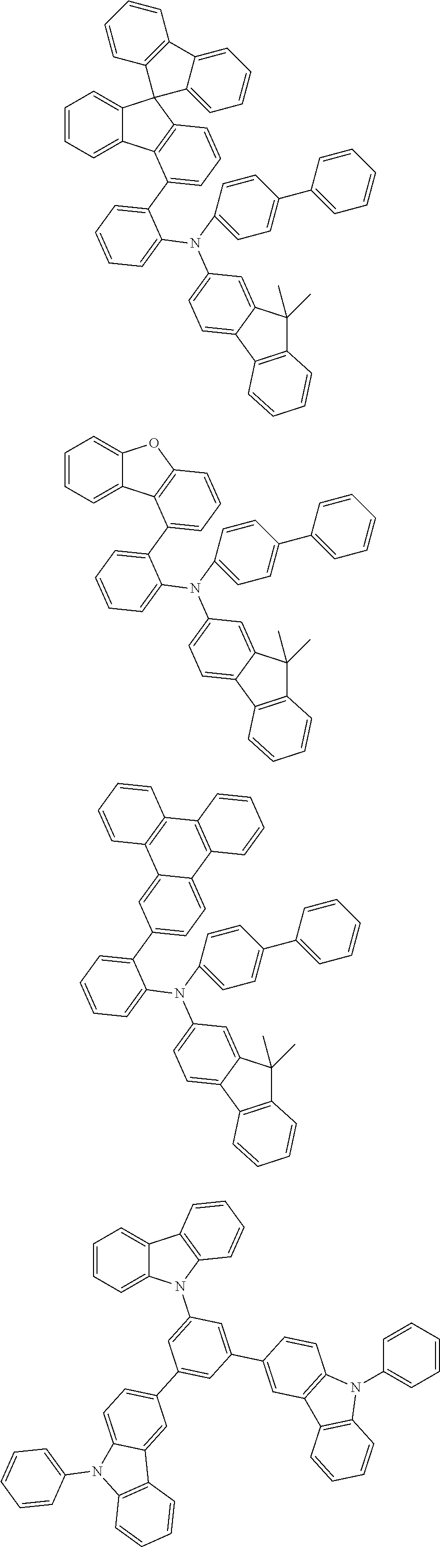

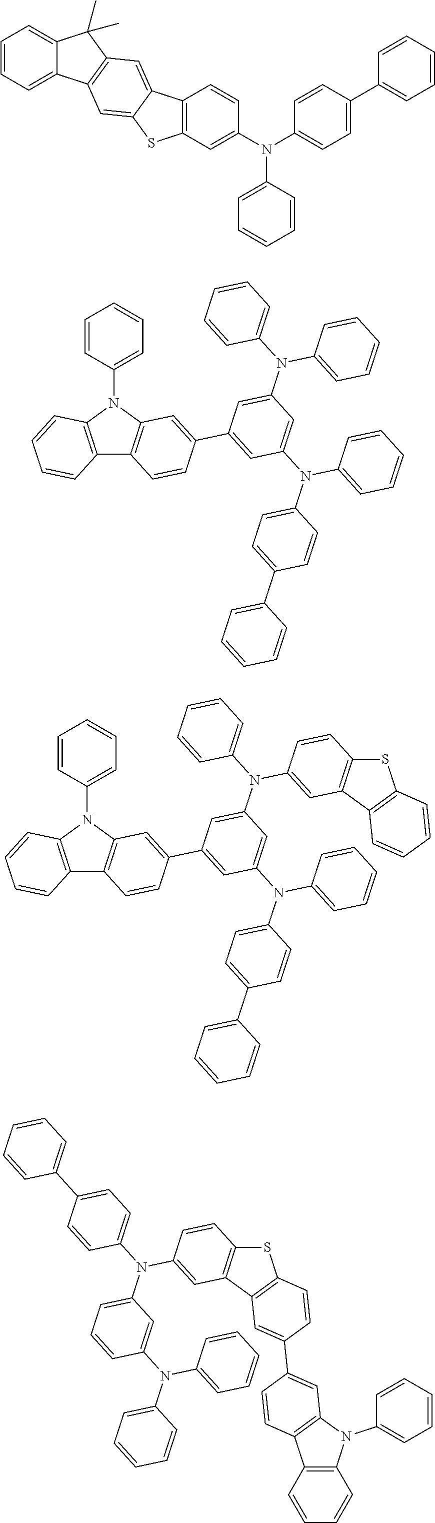

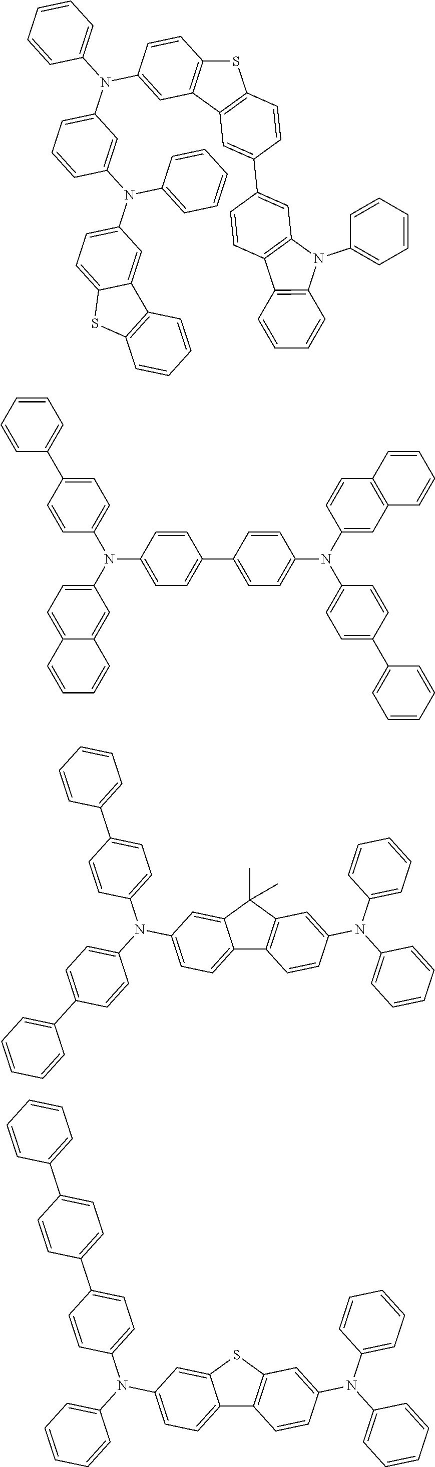

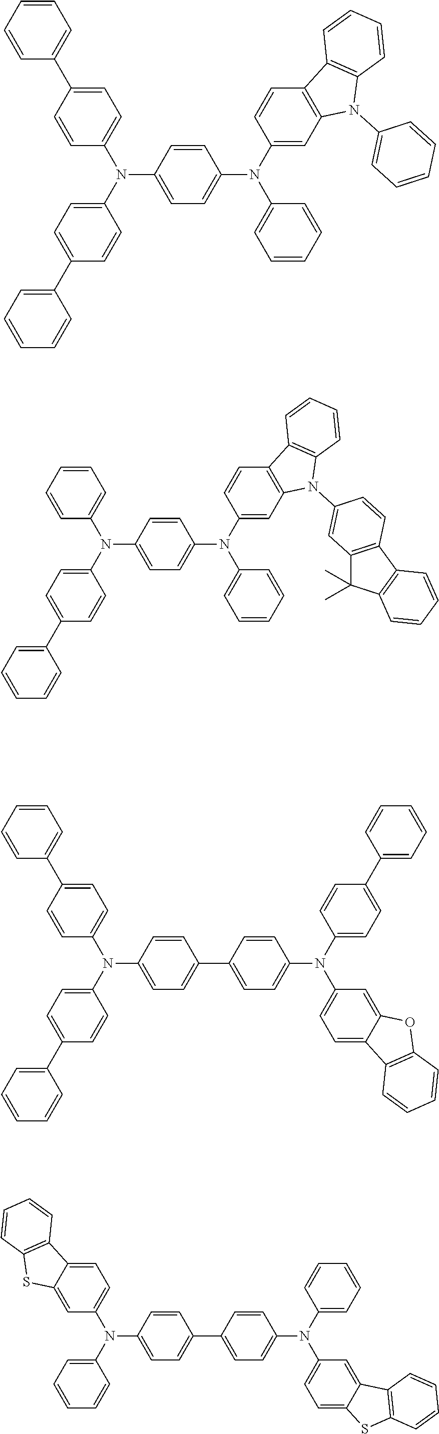 [SMILES: c1ccc(-c2ccc(N(c3ccc(-c4ccccc4)cc3)c3ccc(N(c4ccccc4)c4ccc5c6ccccc6n(-c6ccccc6)c5c4)cc3)cc2)cc1 ; CC1(C)c2ccccc2-c2ccc(-n3c4ccccc4c4ccc(N(c5ccccc5)c5ccc(N(c6ccccc6)c6ccc(-c7ccccc7)cc6)cc5)cc43)cc21 ; c1ccc(-c2ccc(N(c3ccc(-c4ccccc4)cc3)c3ccc(-c4ccc(N(c5ccc(-c6ccccc6)cc5)c5ccc6c(c5)oc5ccccc56)cc4)cc3)cc2)cc1 ; c1ccc(N(c2ccc(-c3ccc(N(c4ccccc4)c4ccc5sc6ccccc6c5c4)cc3)cc2)c2ccc3c(c2)sc2ccccc23)cc1]

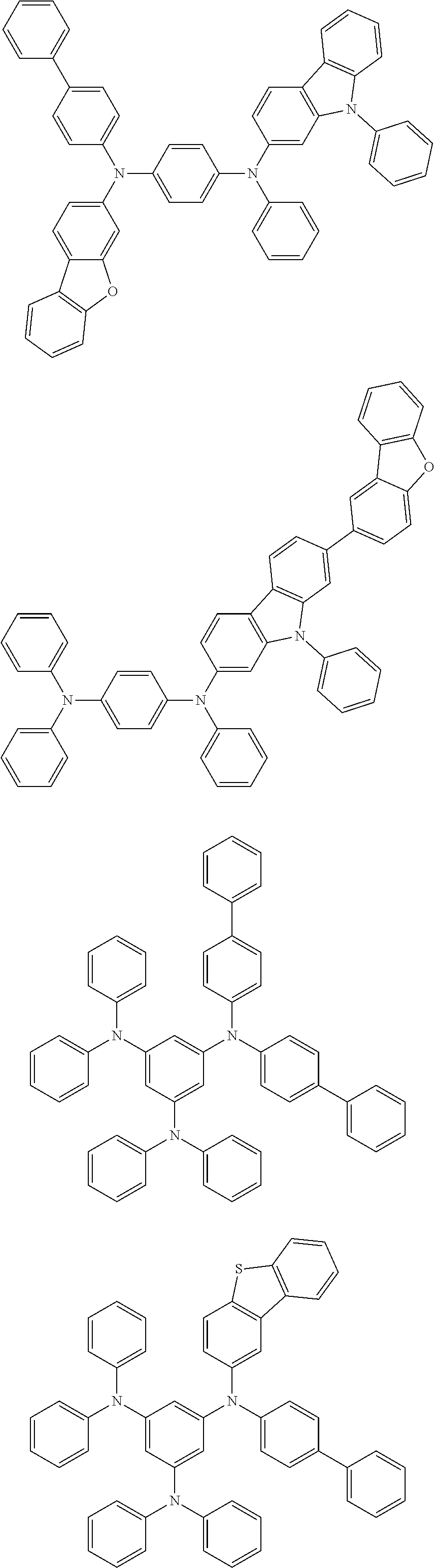 [SMILES: c1ccc(-c2ccc(N(c3ccc(N(c4ccccc4)c4ccc5c6ccccc6n(-c6ccccc6)c5c4)cc3)c3ccc4c(c3)oc3ccccc34)cc2)cc1 ; c1ccc(N(c2ccccc2)c2ccc(N(c3ccccc3)c3ccc4c5ccc(-c6ccc7oc8ccccc8c7c6)cc5n(-c5ccccc5)c4c3)cc2)cc1 ; c1ccc(-c2ccc(N(c3ccc(-c4ccccc4)cc3)c3cc(N(c4ccccc4)c4ccccc4)cc(N(c4ccccc4)c4ccccc4)c3)cc2)cc1 ; c1ccc(-c2ccc(N(c3cc(N(c4ccccc4)c4ccccc4)cc(N(c4ccccc4)c4ccccc4)c3)c3ccc4sc5ccccc5c4c3)cc2)cc1]

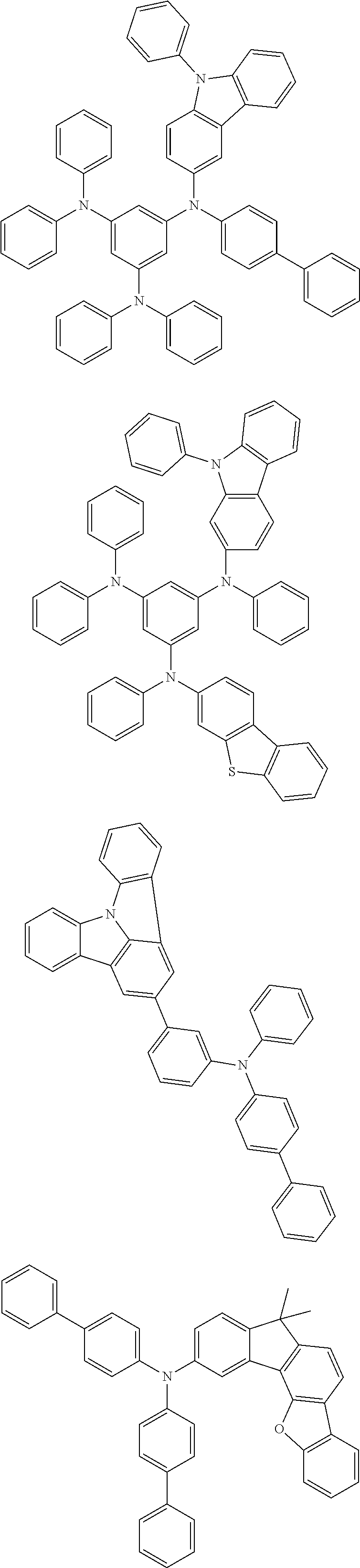 [SMILES: c1ccc(-c2ccc(N(c3cc(N(c4ccccc4)c4ccccc4)cc(N(c4ccccc4)c4ccccc4)c3)c3ccc4c(c3)c3ccccc3n4-c3ccccc3)cc2)cc1 ; c1ccc(N(c2ccccc2)c2cc(N(c3ccccc3)c3ccc4c(c3)sc3ccccc34)cc(N(c3ccccc3)c3ccc4c5ccccc5n(-c5ccccc5)c4c3)c2)cc1 ; c1ccc(-c2ccc(N(c3ccccc3)c3cccc(-c4cc5c6ccccc6n6c7ccccc7c(c4)c56)c3)cc2)cc1 ; CC1(C)c2ccc(N(c3ccc(-c4ccccc4)cc3)c3ccc(-c4ccccc4)cc3)cc2-c2c1ccc1c2oc2ccccc21]

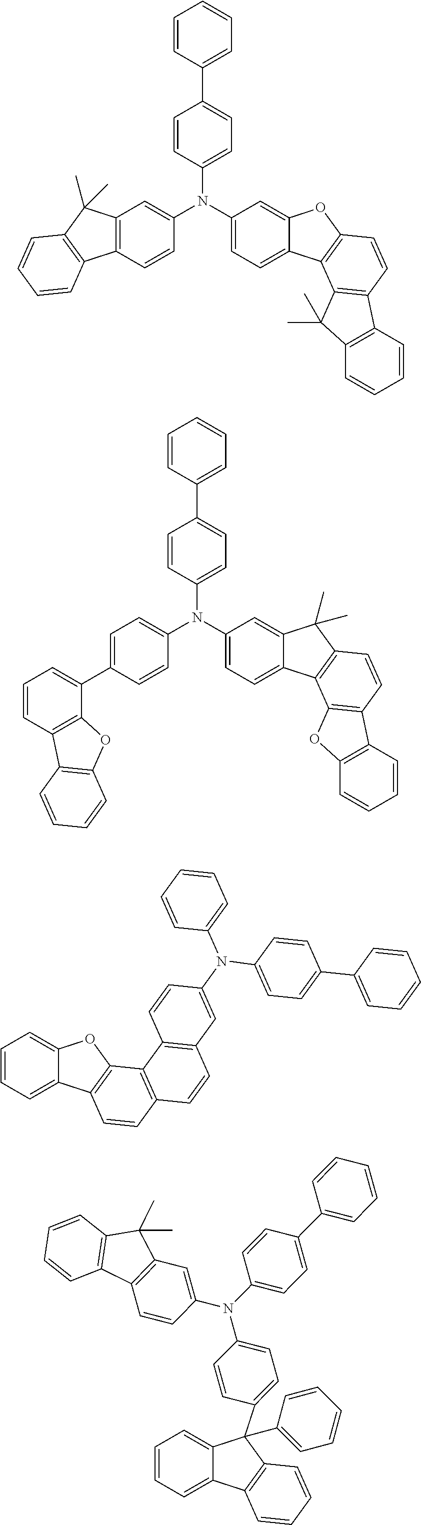 [SMILES: CC1(C)c2ccccc2-c2ccc(N(c3ccc(-c4ccccc4)cc3)c3ccc4c(c3)oc3ccc5c(c34)C(C)(C)c3ccccc3-5)cc21 ; CC1(C)c2cc(N(c3ccc(-c4ccccc4)cc3)c3ccc(-c4cccc5c4oc4ccccc45)cc3)ccc2-c2c1ccc1c2oc2ccccc21 ; c1ccc(-c2ccc(N(c3ccccc3)c3ccc4c(ccc5ccc6c7ccccc7oc6c54)c3)cc2)cc1 ; CC1(C)c2ccccc2-c2ccc(N(c3ccc(-c4ccccc4)cc3)c3ccc(C4(c5ccccc5)c5ccccc5-c5ccccc54)cc3)cc21]

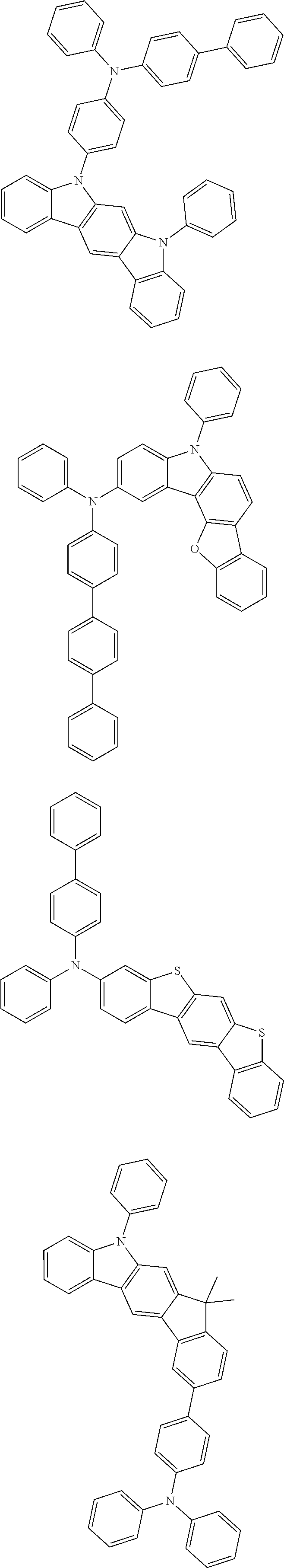

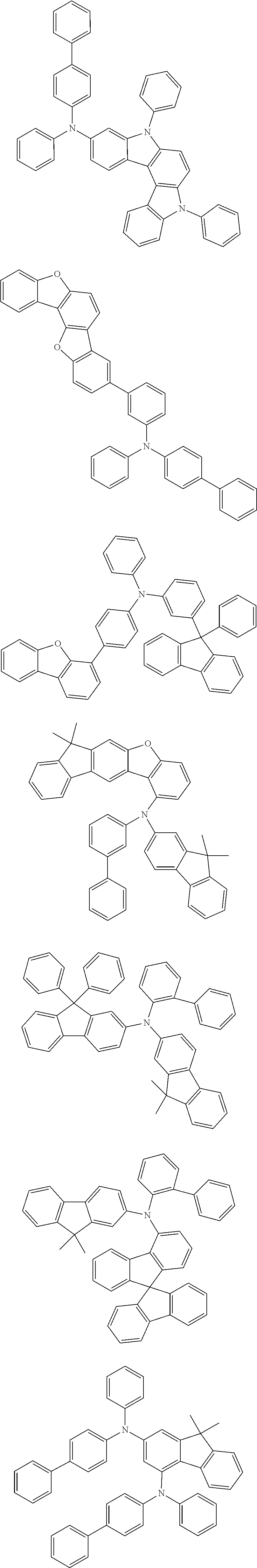

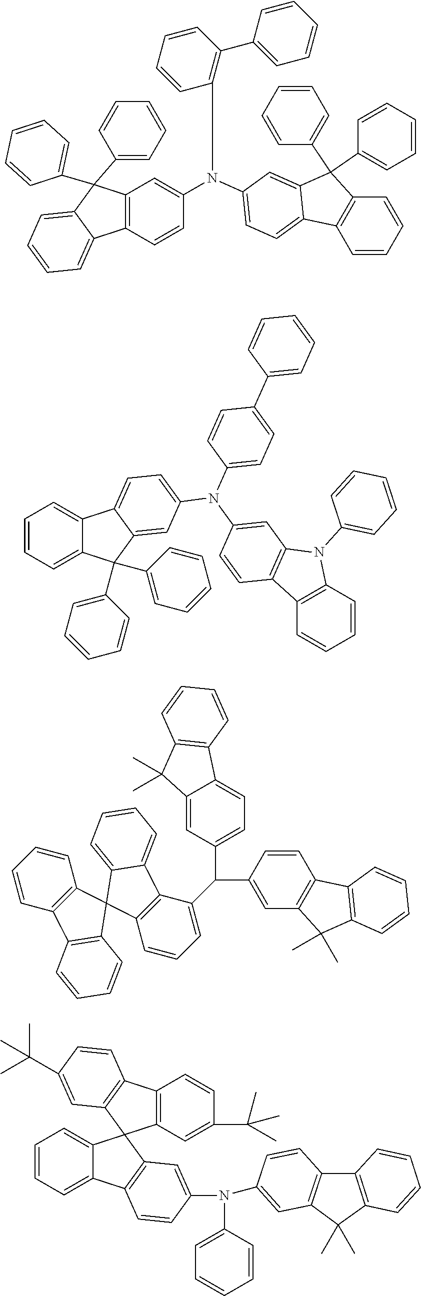

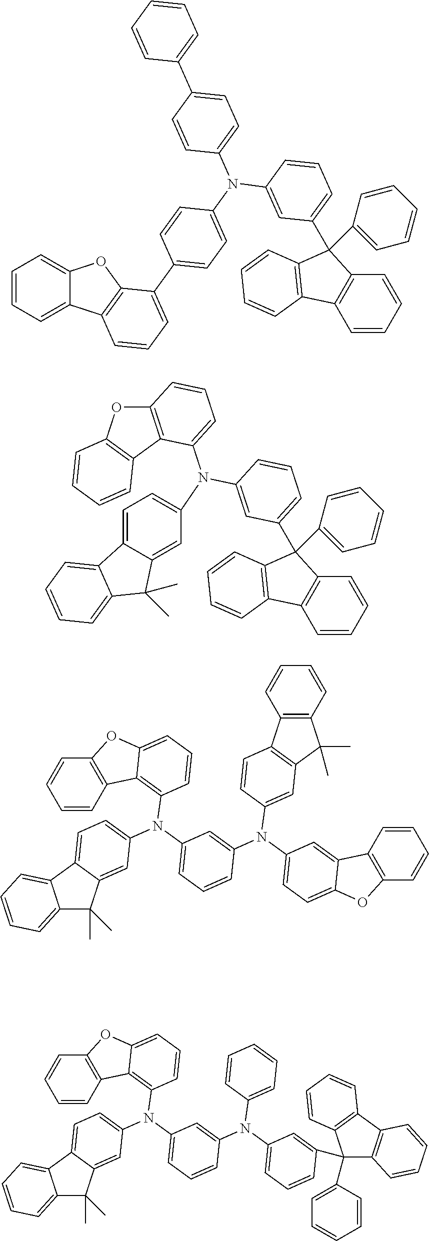

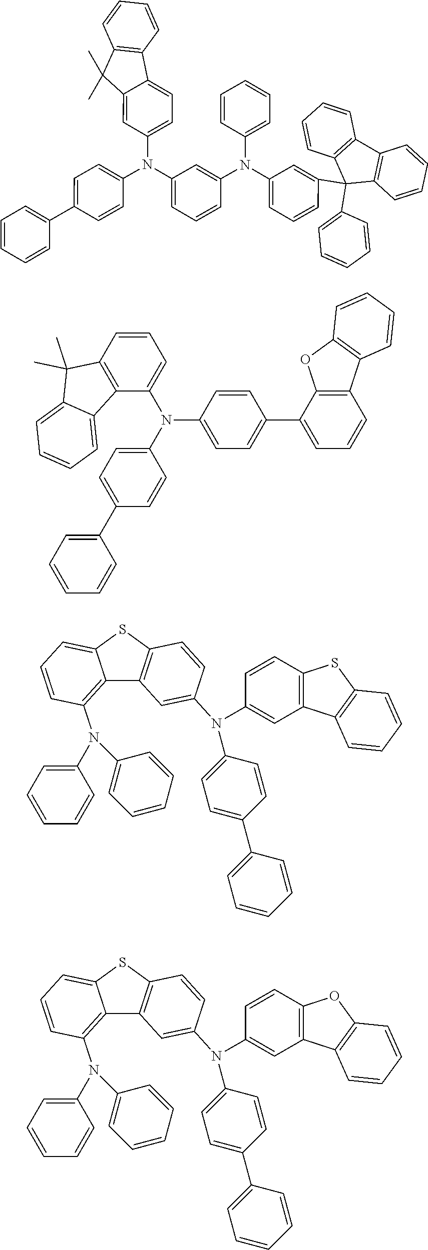

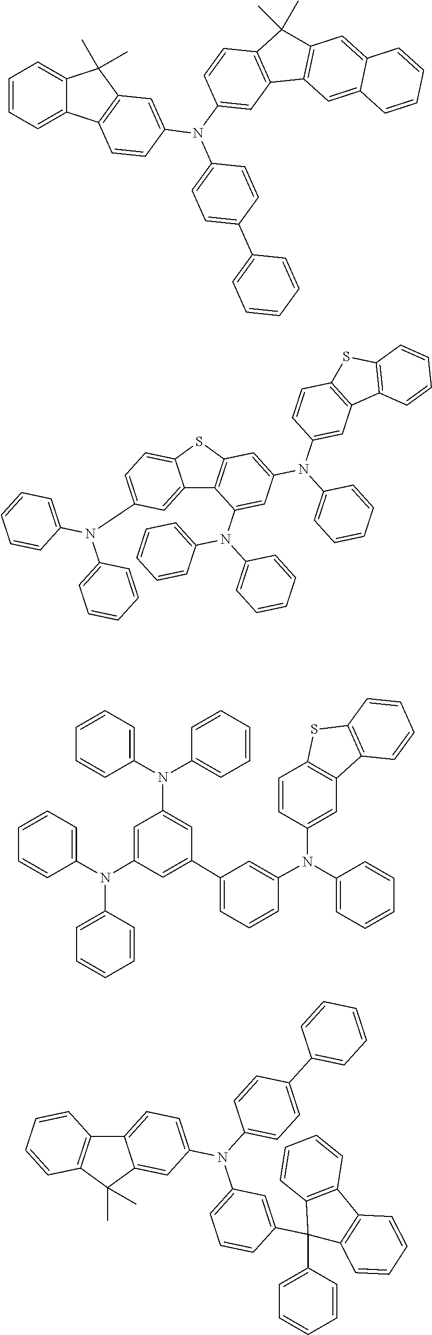 [SMILES: CC1(C)c2ccccc2-c2ccc(N(c3ccc(-c4ccccc4)cc3)c3ccc4c(c3)-c3cc5ccccc5cc3C4(C)C)cc21 ; c1ccc(N(c2cc(N(c3ccccc3)c3ccccc3)c3c(c2)sc2ccc(N(c4ccccc4)c4ccccc4)cc23)c2ccc3sc4ccccc4c3c2)cc1 ; c1ccc(N(c2ccccc2)c2cc(-c3cccc(N(c4ccccc4)c4ccc5sc6ccccc6c5c4)c3)cc(N(c3ccccc3)c3ccccc3)c2)cc1 ; CC1(C)c2ccccc2-c2ccc(N(c3ccc(-c4ccccc4)cc3)c3cccc(C4(c5ccccc5)c5ccccc5-c5ccccc54)c3)cc21]

In the hole transport region, in addition to the compounds described above, known compounds disclosed in U.S. Pat. No. 5,061,569A, JP1993-009471A, WO1995-009147A1, JP1995-126615A, JP1998-095973A, etc. and compounds having a similar structure may also be used.

Also, the charge transport region may be, for example, the electron transport region 150.

The electron transport region 150 may further increase electron injection and/or electron mobility and block holes between the cathode 110 and the light emitting layer 130.

Specifically, the electron transport region 150 may include an electron transport layer between the cathode 110 and the light emitting layer 130, and an electron transport auxiliary layer between the light emitting layer 130 and the electron transport layer, and at least one of the compounds of Group B may be included in at least one of the electron transport layer and the electron transport auxiliary layer.

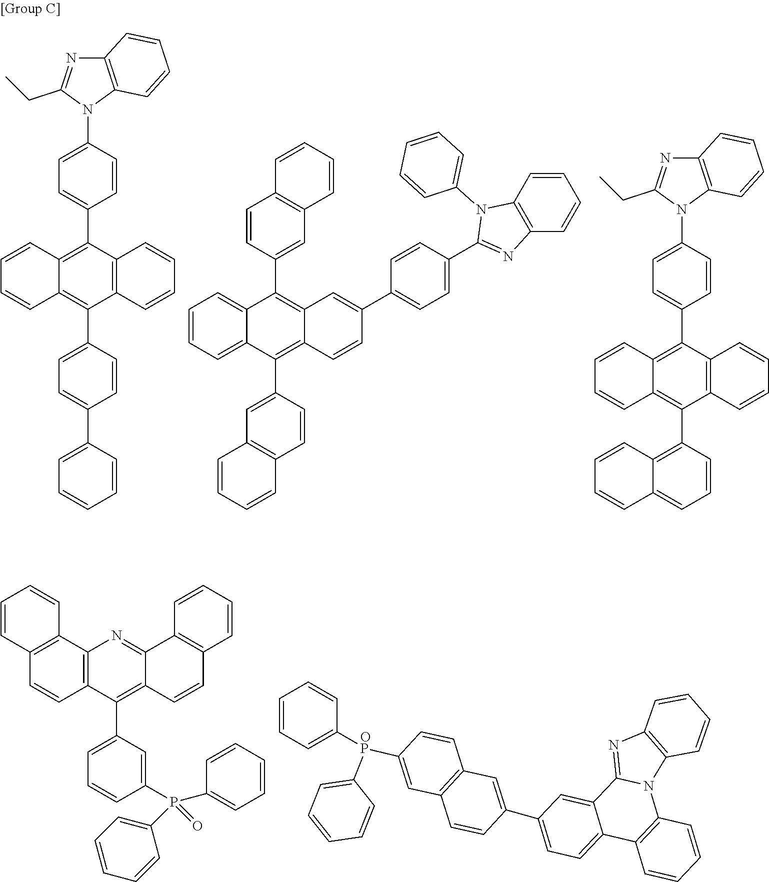

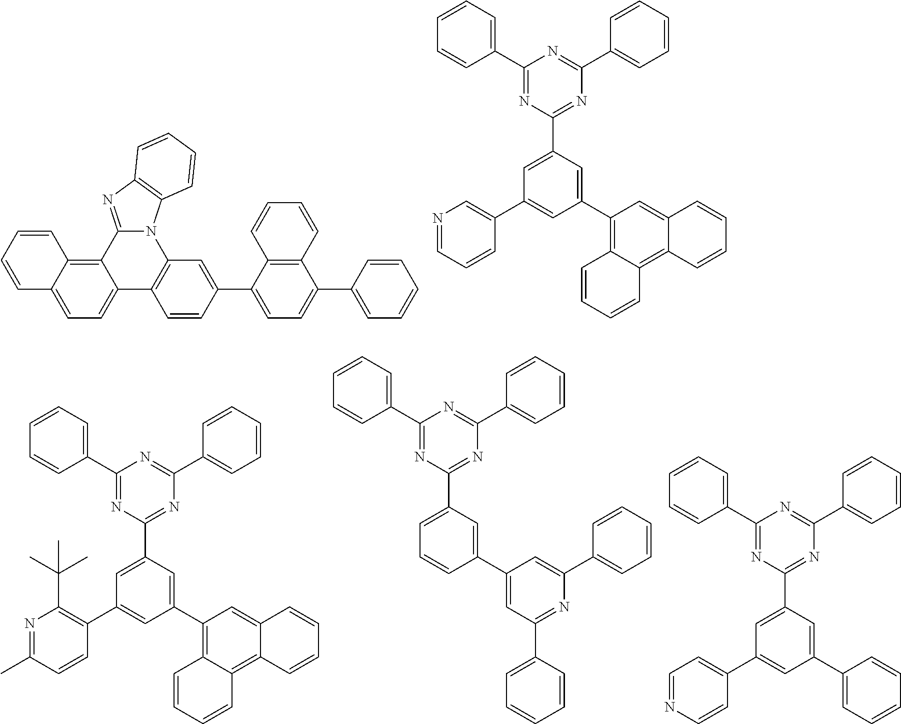

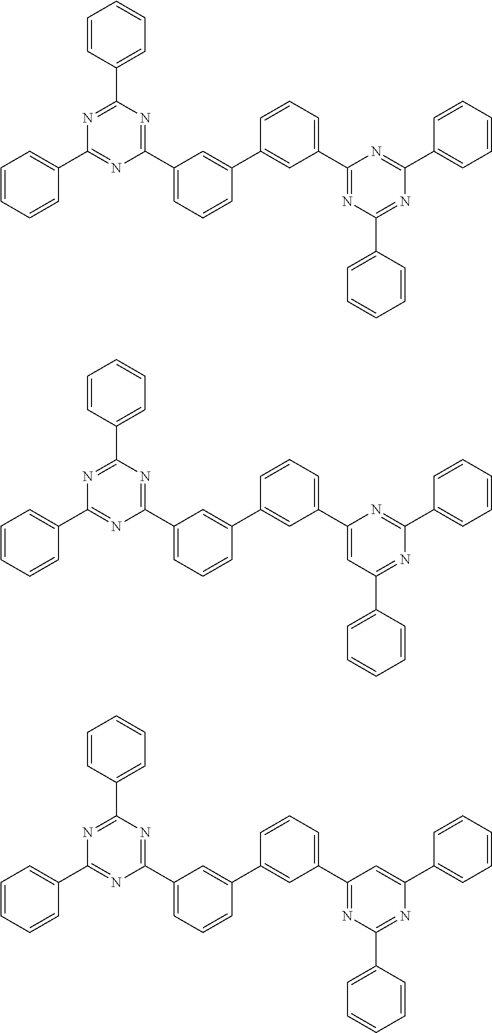 [SMILES: c1ccc(-c2nc(-c3ccccc3)nc(-c3cccc(-c4cccc(-c5nc(-c6ccccc6)nc(-c6ccccc6)n5)c4)c3)n2)cc1 ; c1ccc(-c2cc(-c3cccc(-c4cccc(-c5nc(-c6ccccc6)nc(-c6ccccc6)n5)c4)c3)nc(-c3ccccc3)n2)cc1 ; c1ccc(-c2cc(-c3cccc(-c4cccc(-c5nc(-c6ccccc6)nc(-c6ccccc6)n5)c4)c3)nc(-c3ccccc3)n2)cc1]

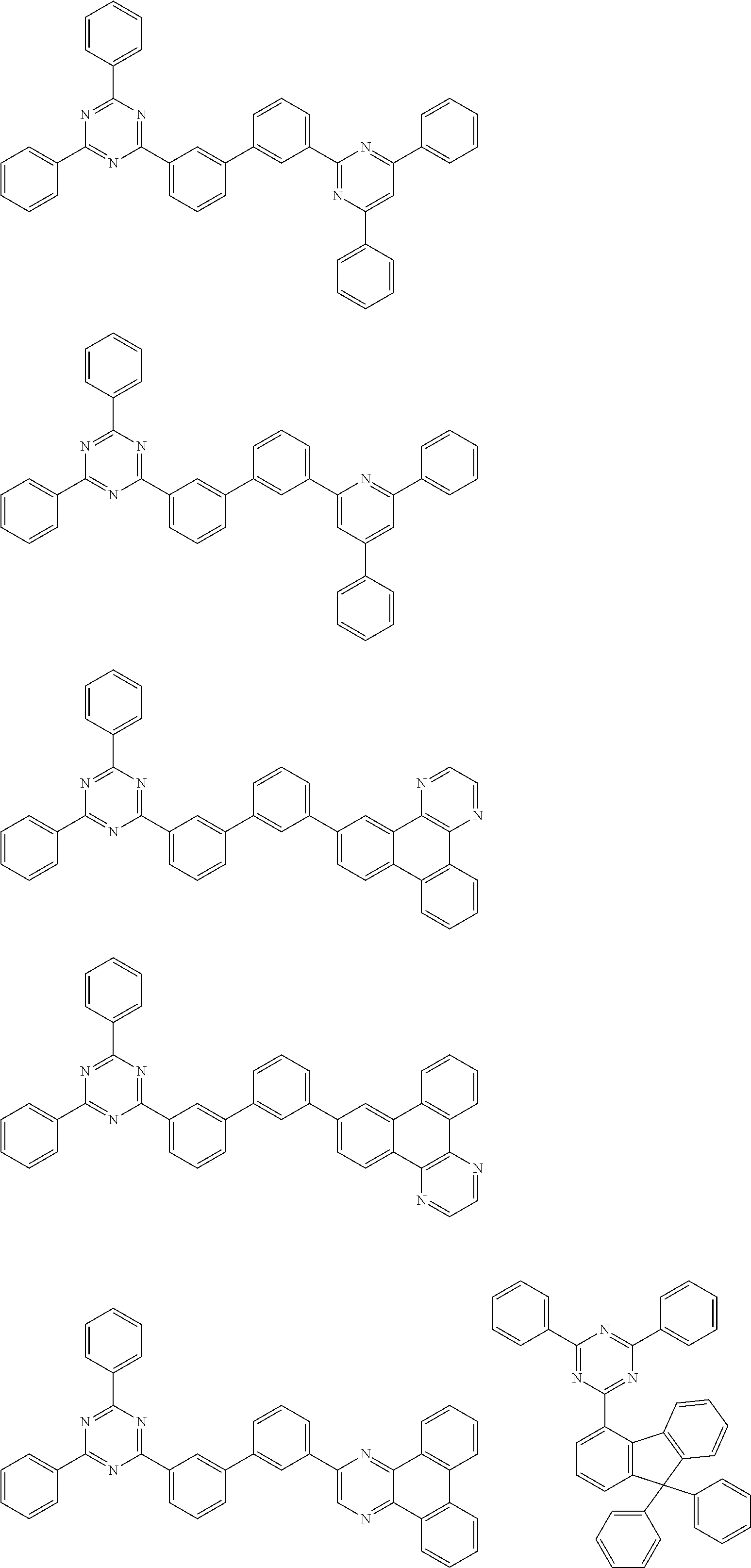

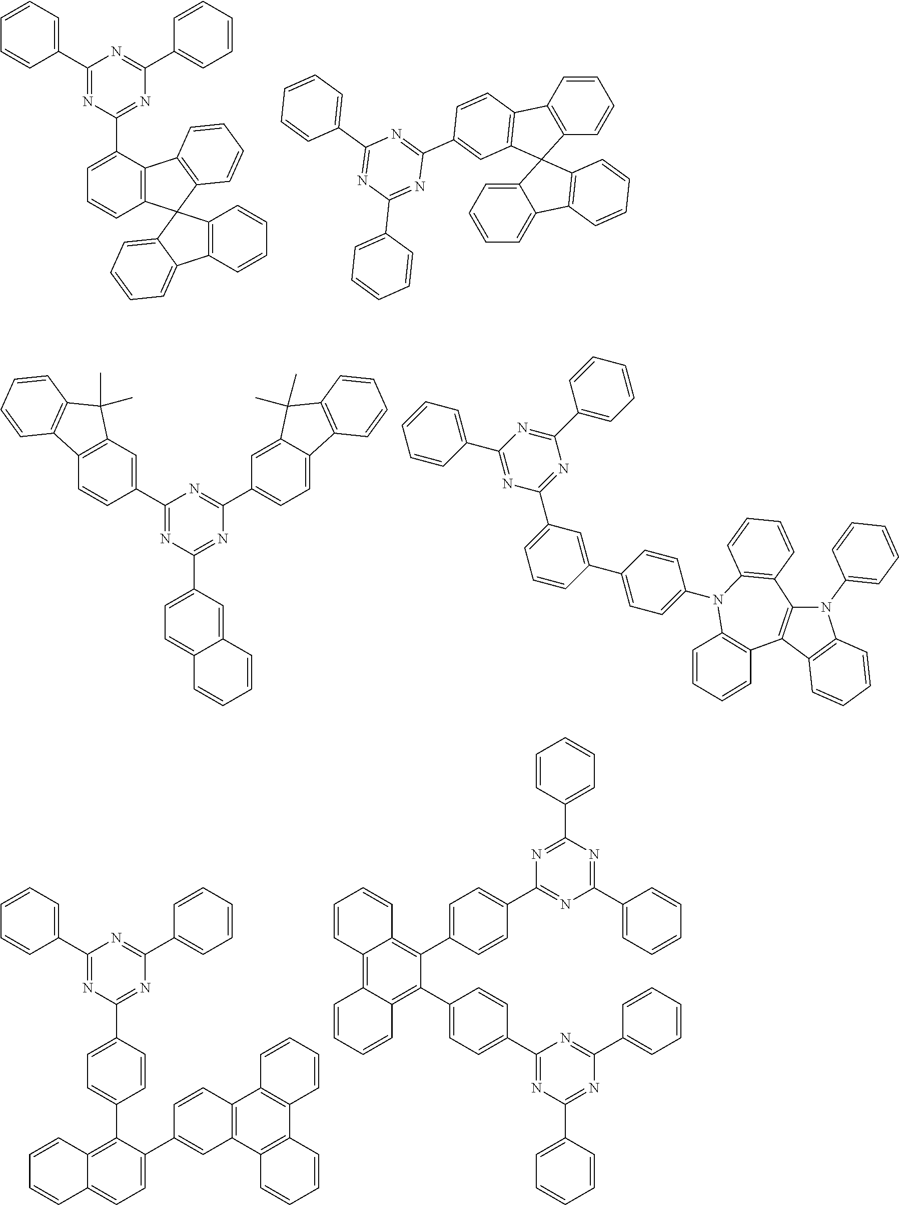

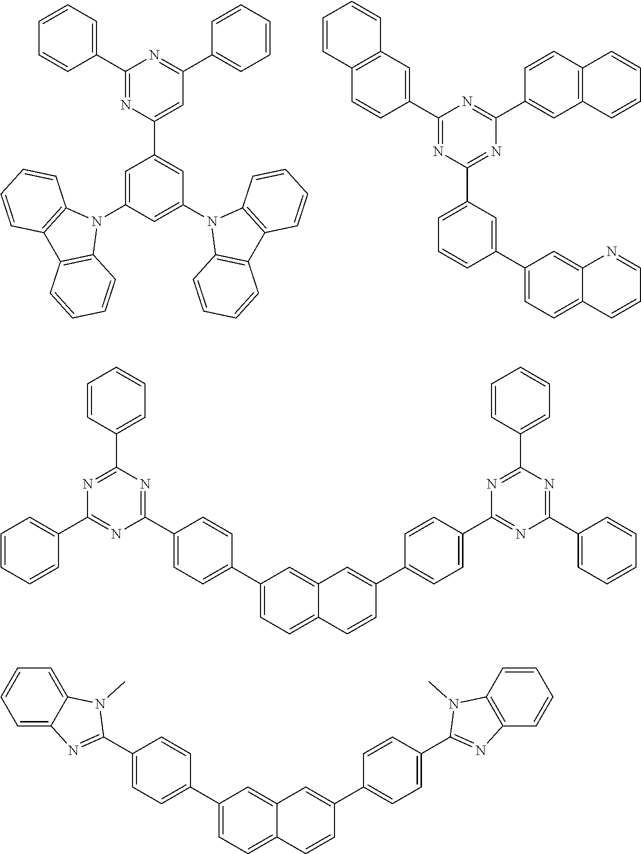 [SMILES: c1ccc(-c2cc(-c3cc(-n4c5ccccc5c5ccccc54)cc(-n4c5ccccc5c5ccccc54)c3)nc(-c3ccccc3)n2)cc1 ; c1cc(-c2ccc3cccnc3c2)cc(-c2nc(-c3ccc4ccccc4c3)nc(-c3ccc4ccccc4c3)n2)c1 ; c1ccc(-c2nc(-c3ccccc3)nc(-c3ccc(-c4ccc5ccc(-c6ccc(-c7nc(-c8ccccc8)nc(-c8ccccc8)n7)cc6)cc5c4)cc3)n2)cc1 ; Cn1c(-c2ccc(-c3ccc4ccc(-c5ccc(-c6nc7ccccc7n6C)cc5)cc4c3)cc2)nc2ccccc21]

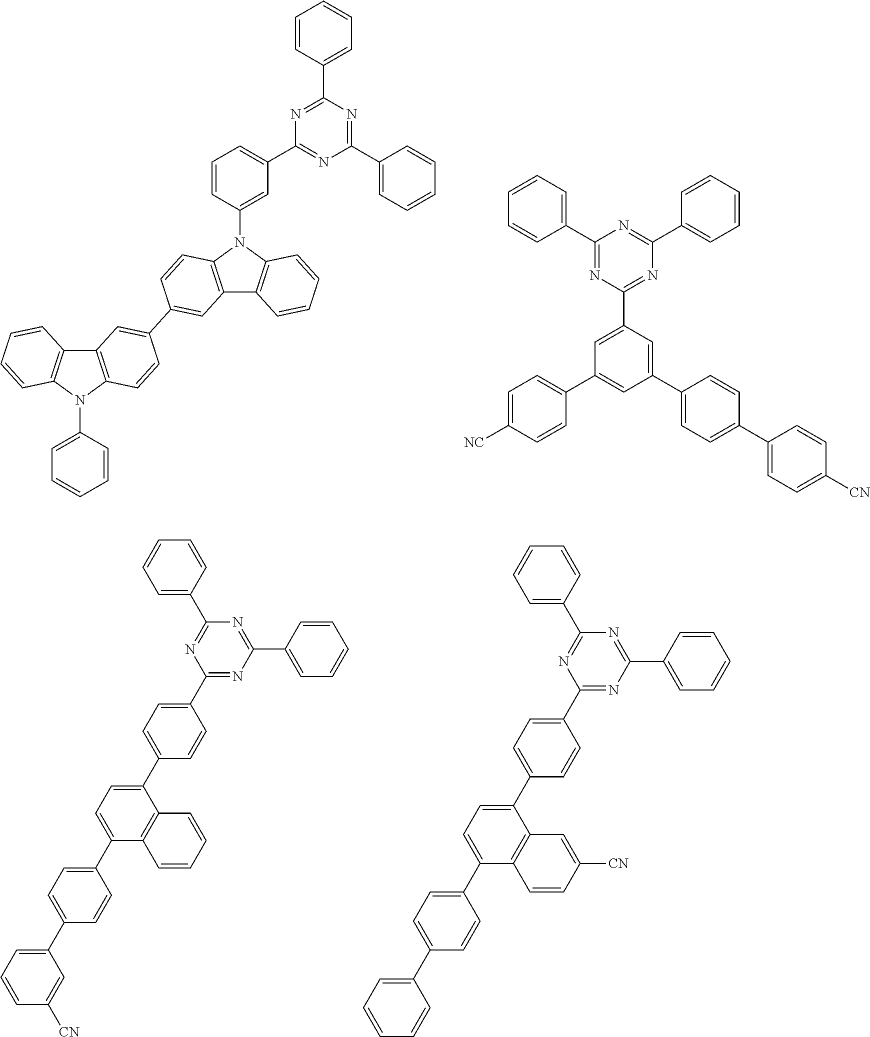 [SMILES: c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; N#Cc1ccc(-c2ccc(-c3cc(-c4ccc(C#N)cc4)cc(-c4nc(-c5ccccc5)nc(-c5ccccc5)n4)c3)cc2)cc1 ; N#Cc1cccc(-c2ccc(-c3ccc(-c4ccc(-c5nc(-c6ccccc6)nc(-c6ccccc6)n5)cc4)c4ccccc34)cc2)c1 ; N#Cc1ccc2c(-c3ccc(-c4ccccc4)cc3)ccc(-c3ccc(-c4nc(-c5ccccc5)nc(-c5ccccc5)n4)cc3)c2c1]

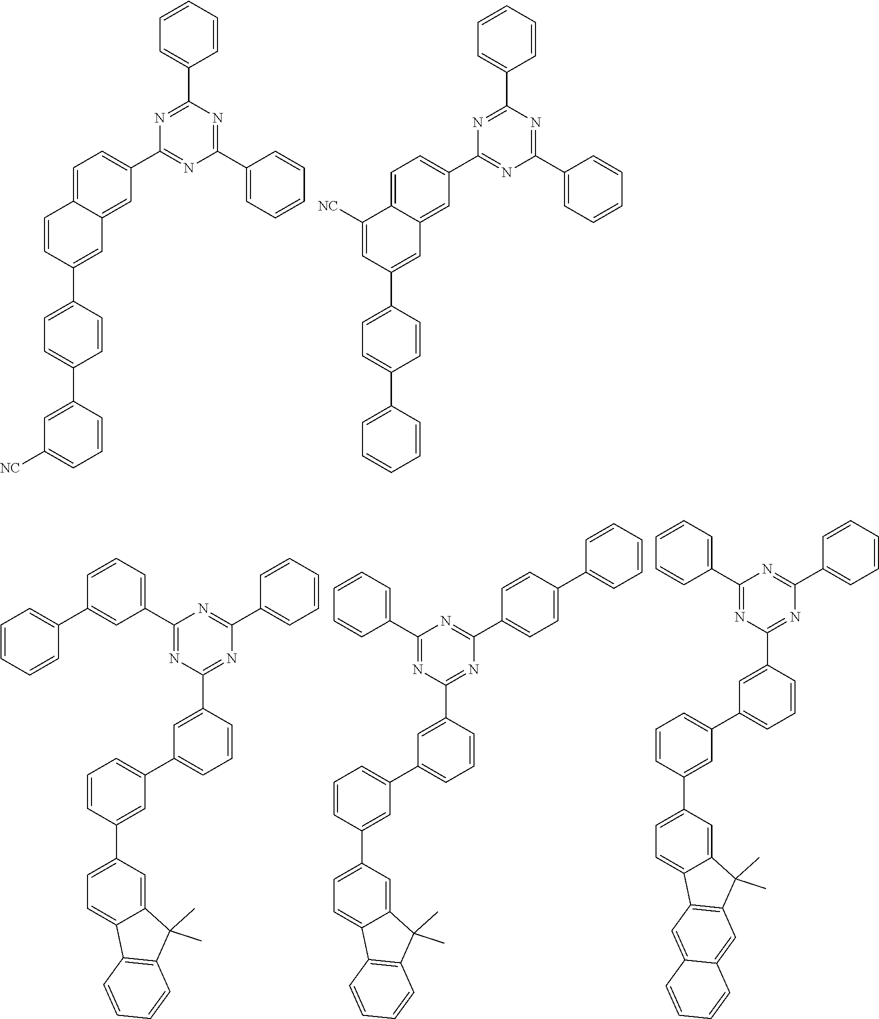

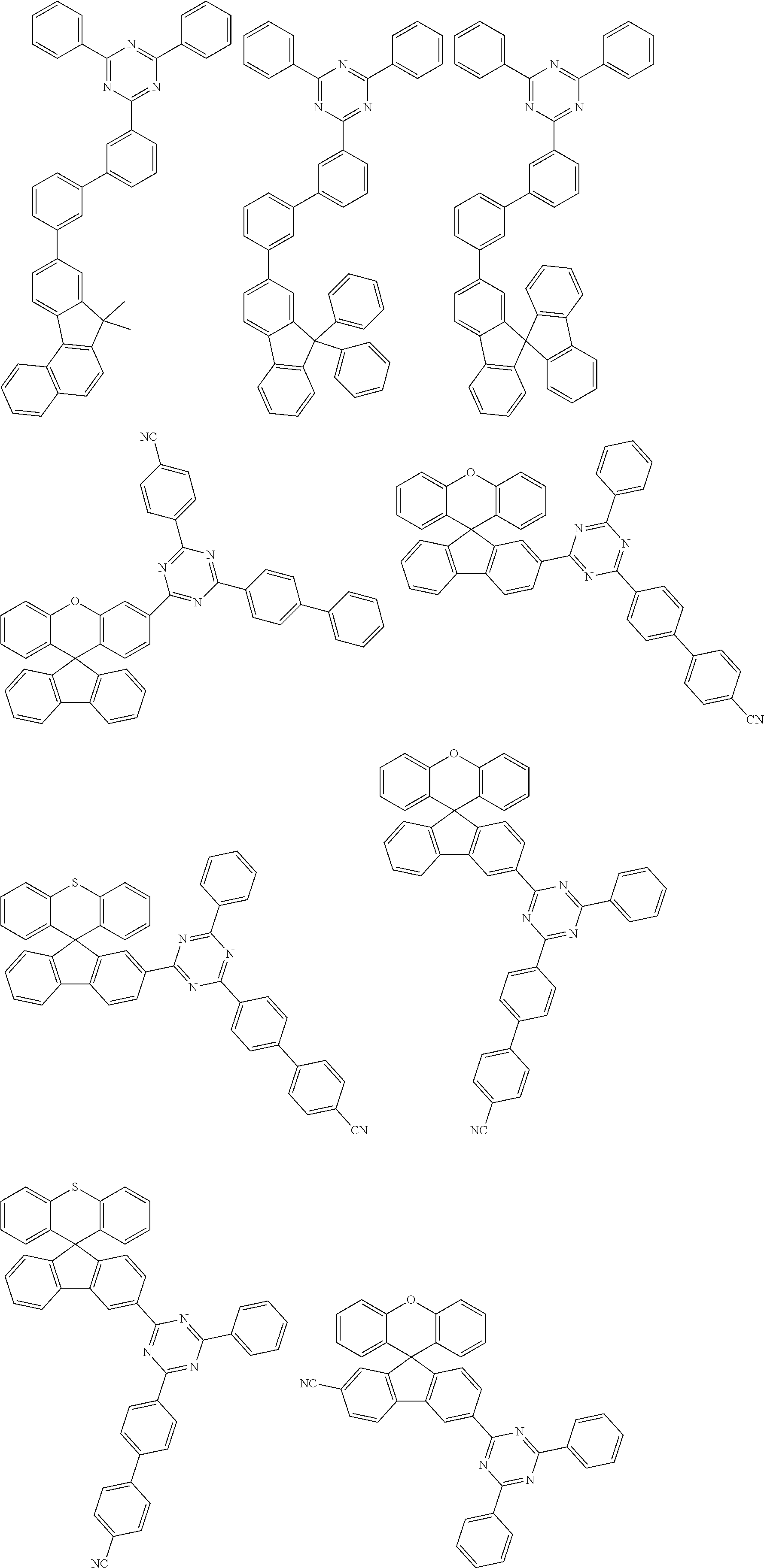

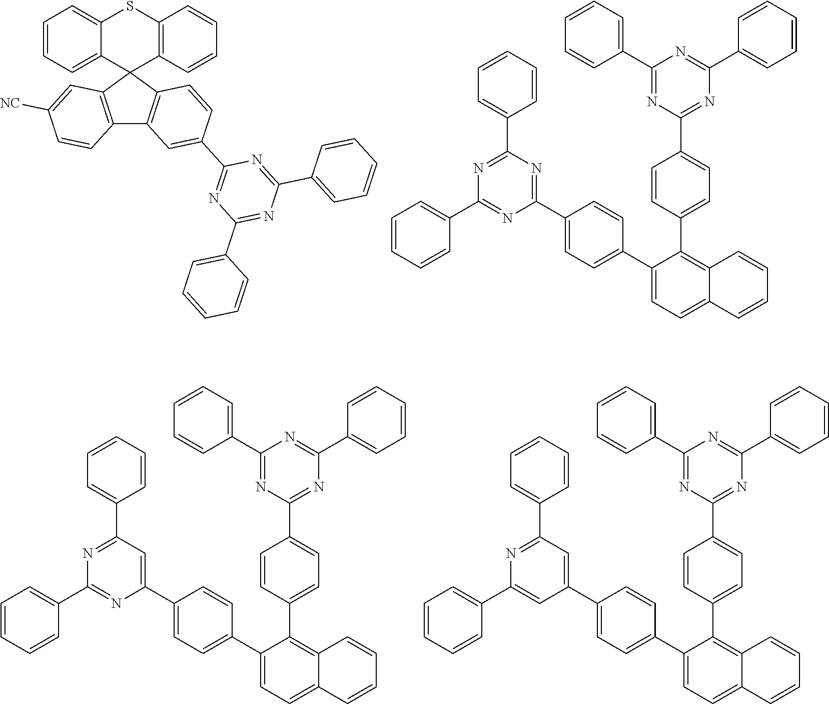

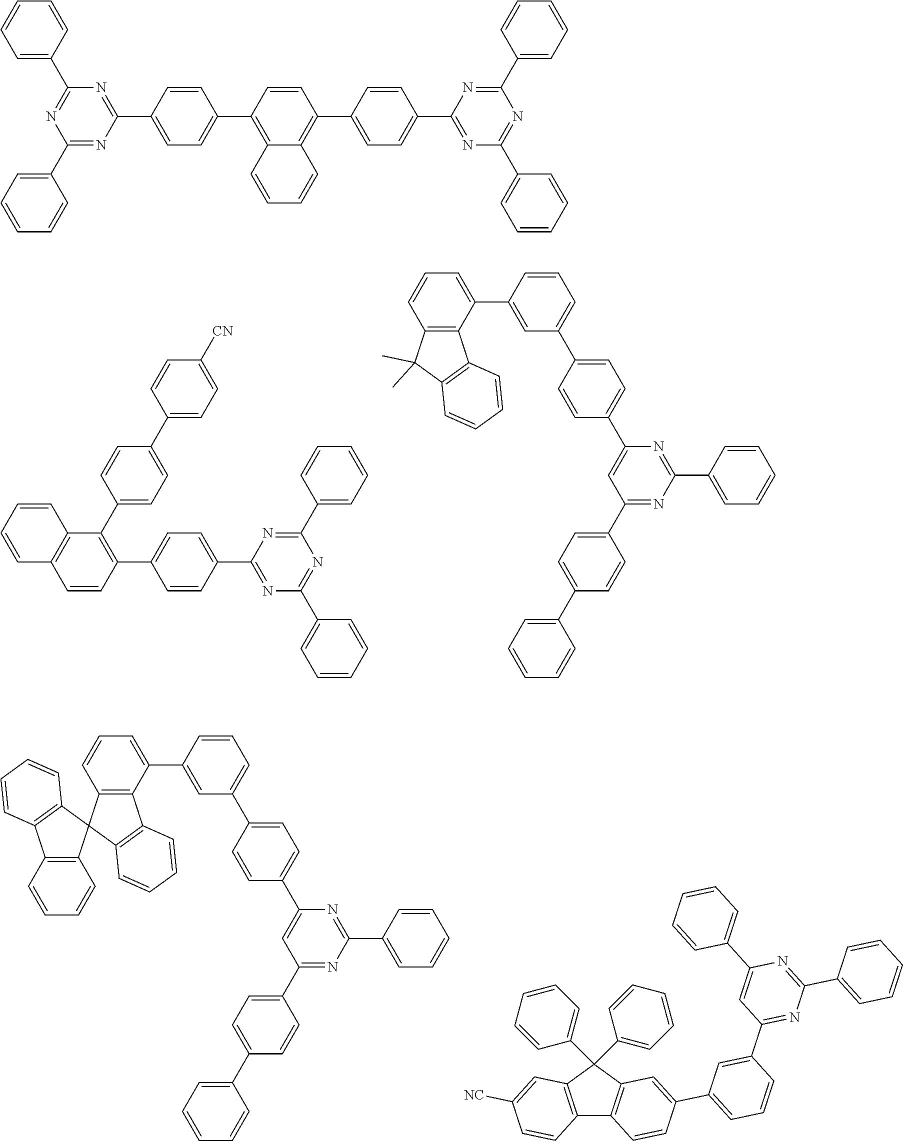

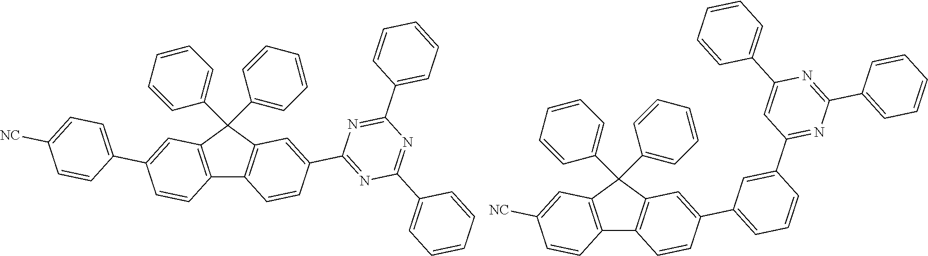

An embodiment of the present invention may provide an organic light emitting diode including the light emitting layer as the organic layer.

Another embodiment of the present invention may provide an organic light emitting diode including a light emitting layer and a hole transport region as the organic layer.

Another embodiment of the present invention may provide an organic light emitting diode including a light emitting layer and an electron transport region as the organic layer.

Another embodiment of the present invention may provide an organic light emitting diode including a hole transport region 140 and an electron transport region 150 in addition to the light emitting layer 130 as the organic layer 105, as shown in FIG. 1.

In another embodiment of the present invention, an organic light emitting diode may further include an electron injection layer (not shown), a hole injection layer (not shown), etc. in addition to the light emitting layer as the organic layer.

The organic light emitting diodes 100 may be manufactured by forming an anode or a cathode on a substrate, and then forming an organic layer by a dry film method such as vacuum deposition, sputtering, plasma plating and ion plating, and forming a cathode or an anode thereon.

The organic light emitting diode may be applied to an organic light emitting display device.

### DETAILED DESCRIPTION OF THE EMBODIMENTS

Hereinafter, the embodiments are illustrated in more detail with reference to examples. However, these examples are exemplary, and the present scope is not limited thereto.

Hereinafter, starting materials and reactants used in Examples and Synthesis Examples were purchased from Sigma-Aldrich Co. Ltd., TCI Inc., or Tokyo chemical industry as far as there in no particular comment or were synthesized by known methods.

### Preparation of Compound for Organic Optoelectronic Device

### Synthesis Example 1: Synthesis of Compound 1-130

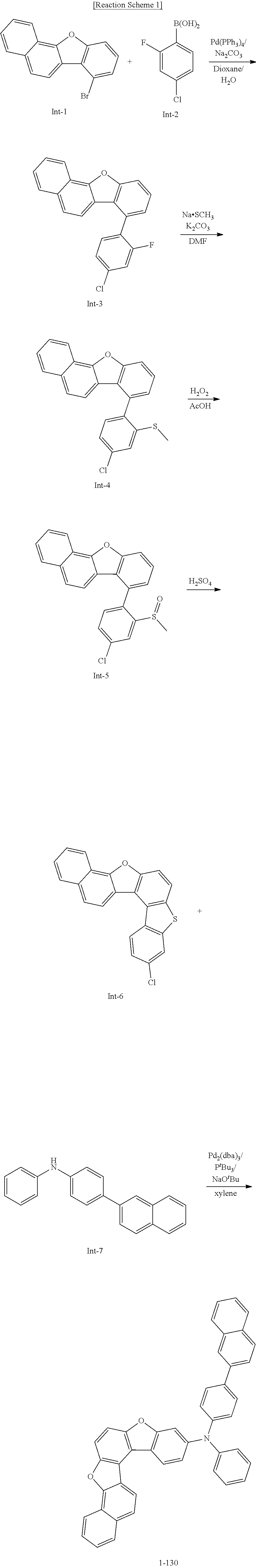

### 1 st Step: Synthesis of Int-3

Int-1 (100 g, 336 mmol) was dissolved in 800 mL of dioxane, and Int-2 (58.7 g, 336 mmol) and tetrakis(triphenylphosphine) palladium (11.7 g, 10.1 mmol) were added thereto and then, stirred. Subsequently, sodium carbonate (89.1 g, 841 mmol) saturated in water was added thereto and then, heated under reflux at 110° C. for 24 hours. When a reaction was completed, after adding water thereto, the reaction solution was extracted with dichloromethane (DCM), treated with magnesium sulfate anhydrous to remove moisture, filtered, and concentrated under a reduced pressure. The obtained residue was separated and purified through flash column chromatography to obtain 100.3 g (86%) of Int-3.

### 2nd Step: Synthesis of Int-4

In a 1000 mL round-bottomed flask, after adding 100.3 g (289 mmol) of Int-3 to 550 mL of N,N-dimethylformamide, an internal temperature thereof was set at 0° C. Subsequently, 22.4 g (303.8 mmol) of sodium thiomethoxide (CAS No.: 5188-07-8) and 59.9 g (433.98 mmol) of potassium carbonate were slowly added thereto. Herein, the internal temperature of 0° C. was maintained. Then, the flask was heated to 80° C. under a nitrogen atmosphere. After 16 hours, the reaction solution was cooled, and after adding ethylacetate and water thereto and then, stirred the mixture, an organic layer therefrom was depressurized and treated through column chromatograph to obtain 85 g (a yield: 78%) of Int-4.

### 3rd Step: Synthesis of Int-5

85 g (227 mmol) of Int-4 was added to 450 mL of acetic acid, and an internal temperature thereof was set at 0° C. Subsequently, 50 ml of hydrogen peroxide was slowly added thereto. Herein, the internal temperature was maintained at 0° C. After stirring at room temperature for 12 hours, the reaction solution was put in ice water and then, extracted with dichloromethane (DCM), treated with magnesium sulfate anhydrous to remove moisture, filtered, and concentrated under a reduced pressure concentrate to obtain 84 g (a yield: 95%) of Int-5.

### 4th Step: Synthesis of Int-6

After adding 84 g (214.9 mmol) of Int-5 to 500 mL of sulfuric acid and then, stirring the mixture at room temperature for 20 hours, the reaction solution was put in ice water and then, adjusted into pH 9 by using a NaOH aqueous solution. Subsequently, the reaction solution was extracted with dichloromethane (DCM), treated with magnesium sulfate anhydrous to remove moisture, filtered under a reduced pressure, and concentrated to obtain 59 g (a yield: 77%) of Int-6.

### 5th Step: Synthesis of Compound 1-130

3.0 g (8.42 mmol) of Int-6, 2.59 g (8.84 mmol) of Int-7, 2.43 g (25.25 mmol) of sodium t-butoxide, and 0.84 g (0.84 mmol) of tri-tert-butylphosphine were dissolved in 50 m1 of xylene, and 0.39 g (0.42 mmol) of Pd₂(dba)₃ was added thereto and then, stirred under reflux for 12 hours under a nitrogen atmosphere. When a reaction was completed, after extracting with xylene and distilled water, an organic layer obtained therefrom was dried with magnesium sulfate anhydrous and filtered, and a filter therefrom was concentrated under a reduced pressure. A product therefrom was purified through silica gel column chromatography with normal hexane/dichloromethane (in a volume ratio of 2:1) to obtain 3.78 g (a yield: 73%) of Compound 1-130.

calcd. C44H27NOS: C, 85.55; H, 4.41; N, 2.27; O, 2.59; S, 5.19 found: C, 85.55; H, 4.41; N, 2.27; O, 2.59; S, 5.19

### Synthesis Example 2: Synthesis of Int-12 to Int-39

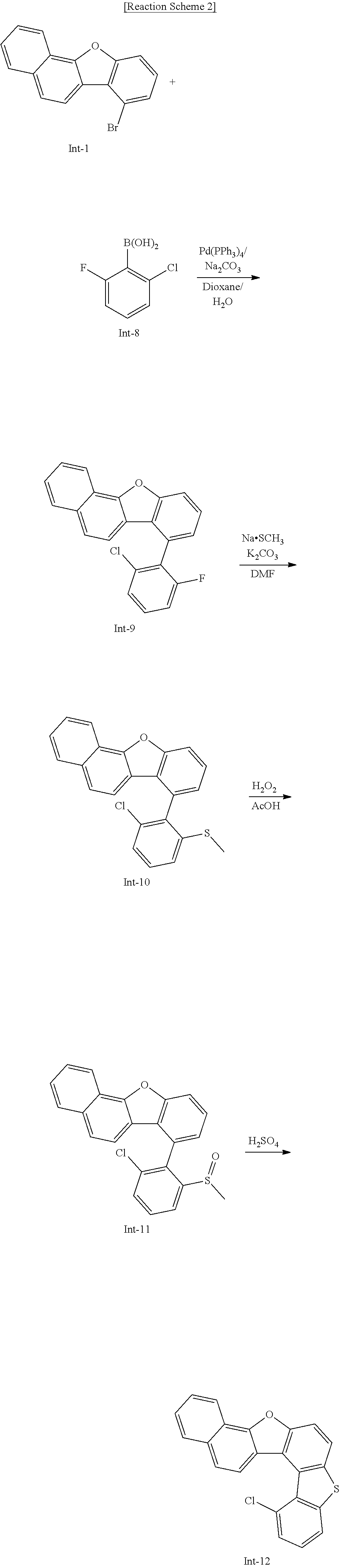

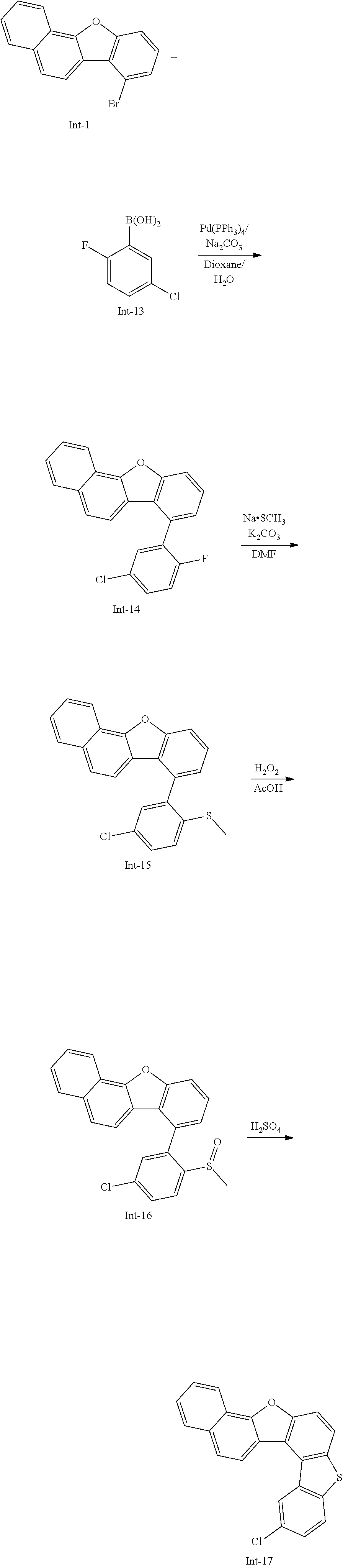

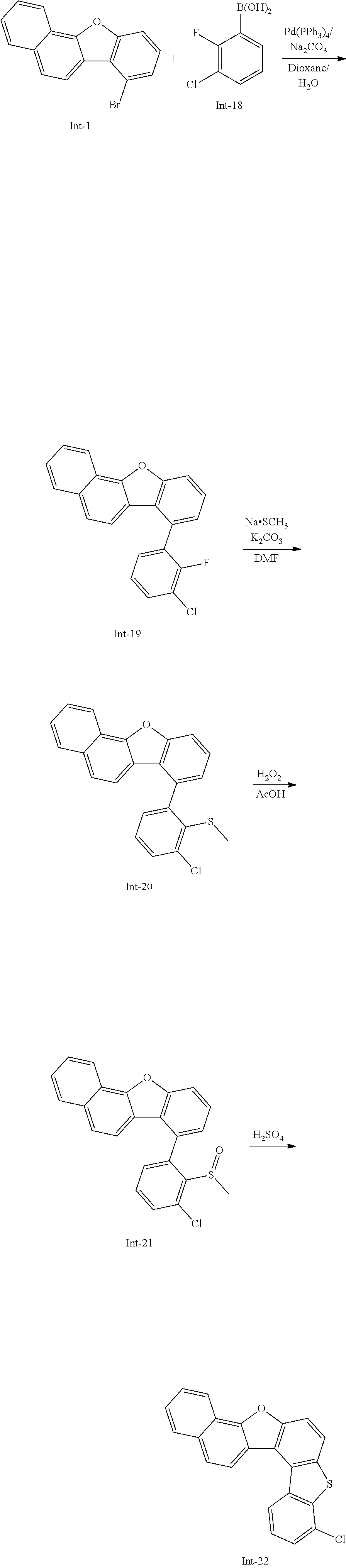

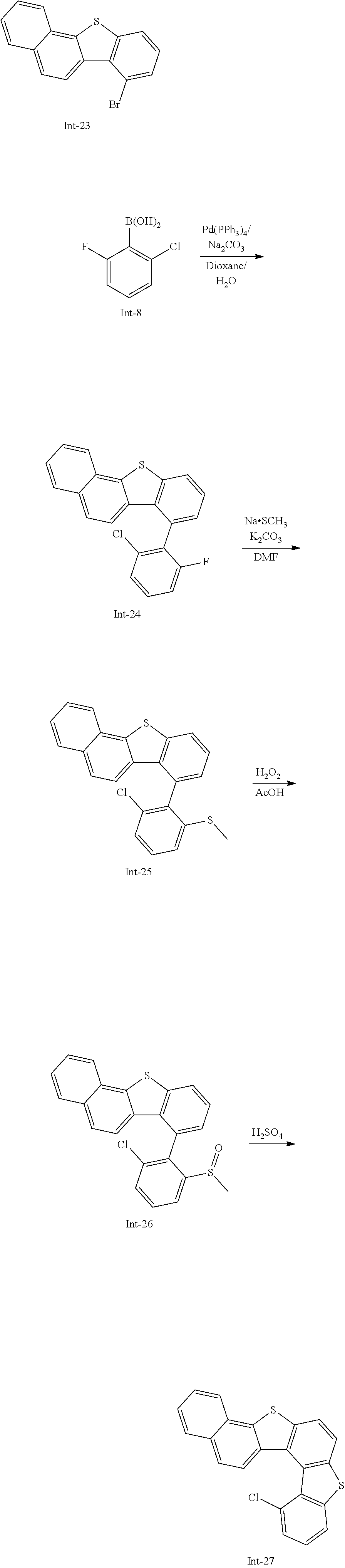

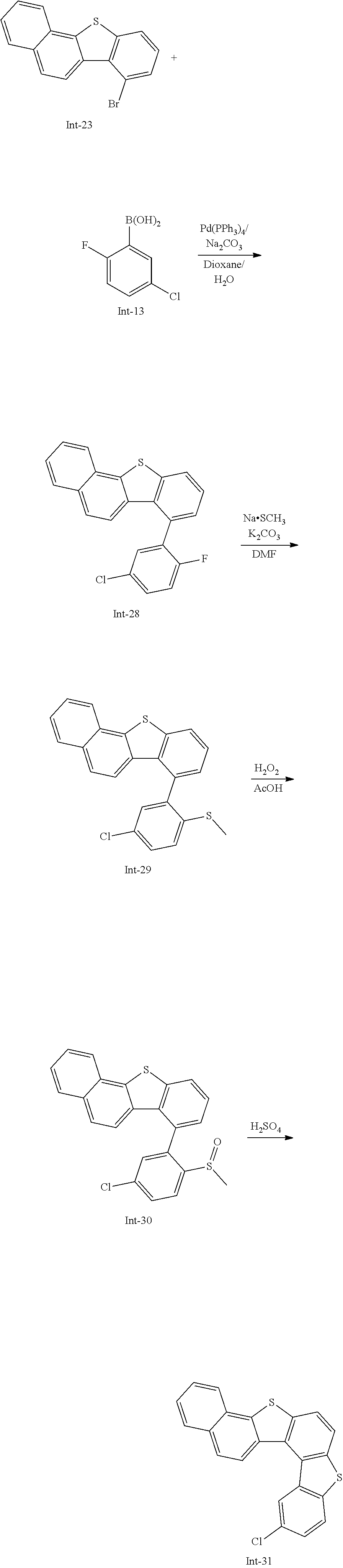

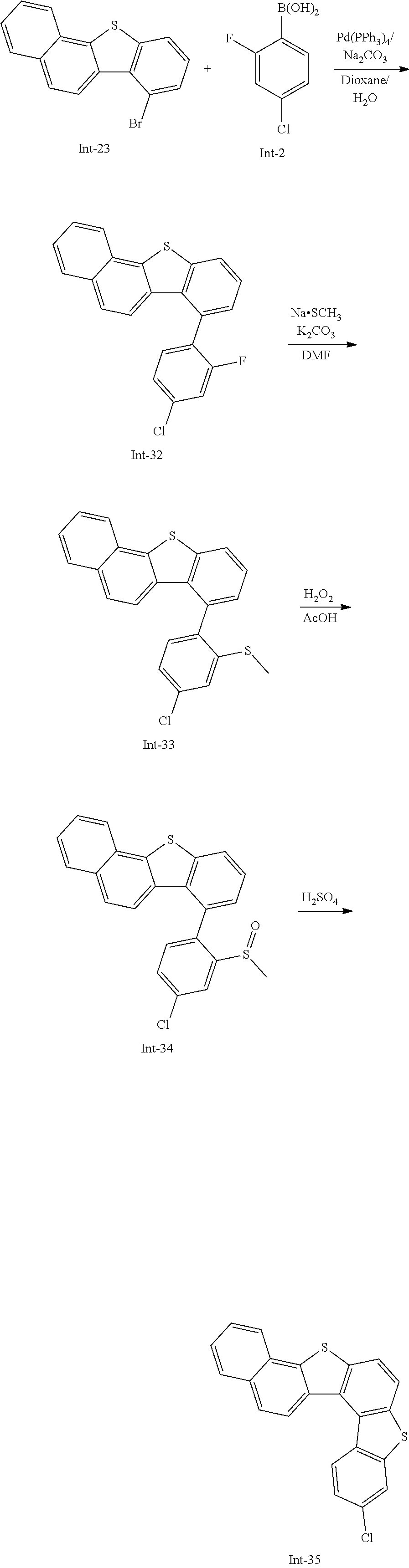

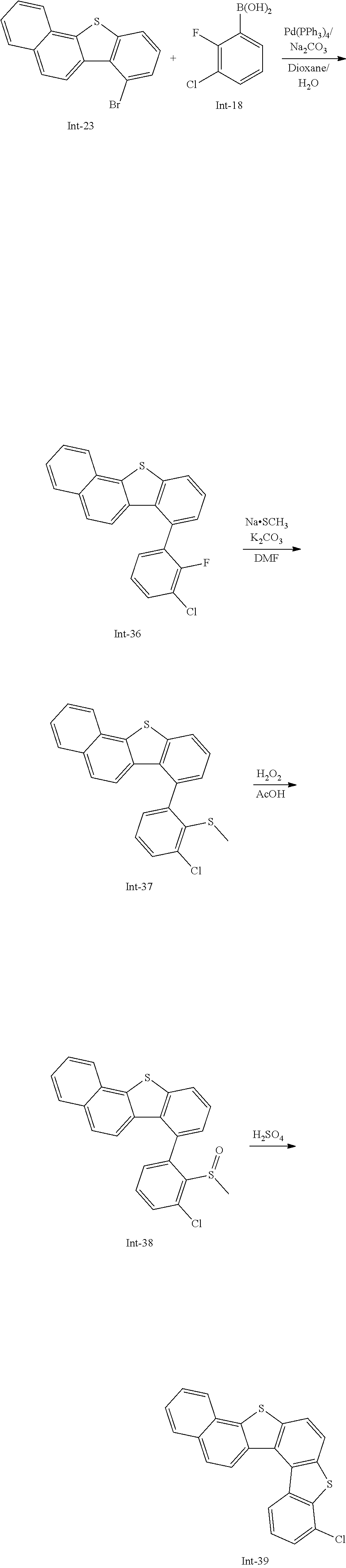

Int-12, Int-17, and Int-22 were respectively synthesized in the same manner as in the 1st to 5th steps of the method of Synthesis Example 1 except that Int-8, Int-13, and Int-18 were respectively used instead of Int-2 of Synthesis Example 1.

In addition, Int-27, Int-31, Int-35, and Int-39 were respectively synthesized in the same manner as in the 1st to 5th steps of the method of Synthesis Example 1 except that Int-23 instead of Int-1 of Synthesis Example 1 was used, and Int-8, Int-13, Int-2, and Int-18 were respectively used.

### Synthesis Example 3: Synthesis of Int-40 to Int-44

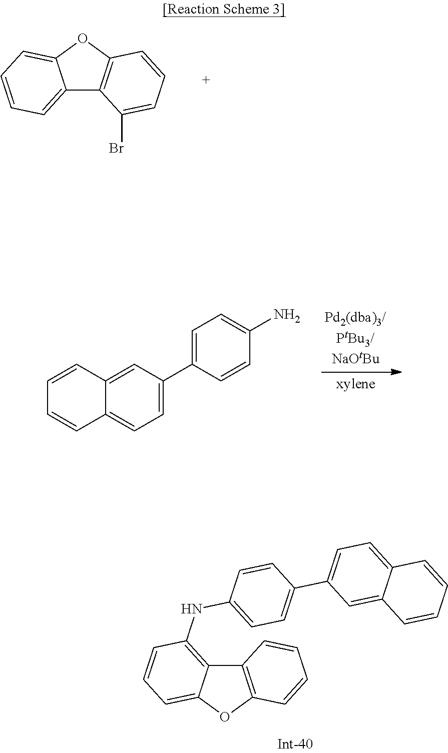 [SMILES: Brc1cccc2oc3ccccc3c12 ; Nc1ccc(-c2ccc3ccccc3c2)cc1 ; c1ccc2cc(-c3ccc(Nc4cccc5oc6ccccc6c45)cc3)ccc2c1]

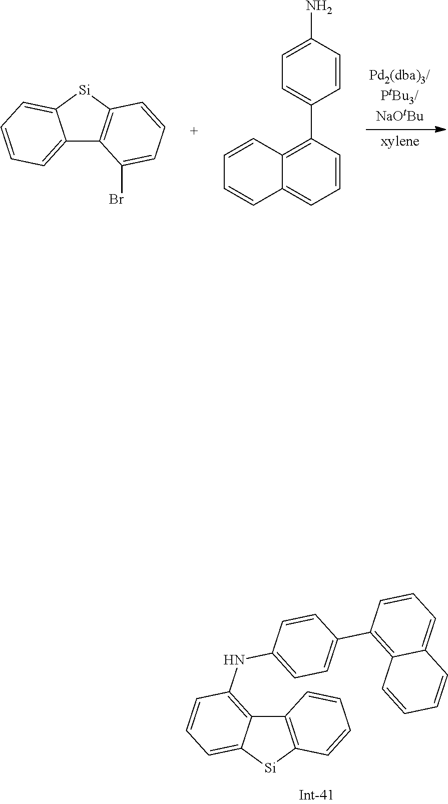

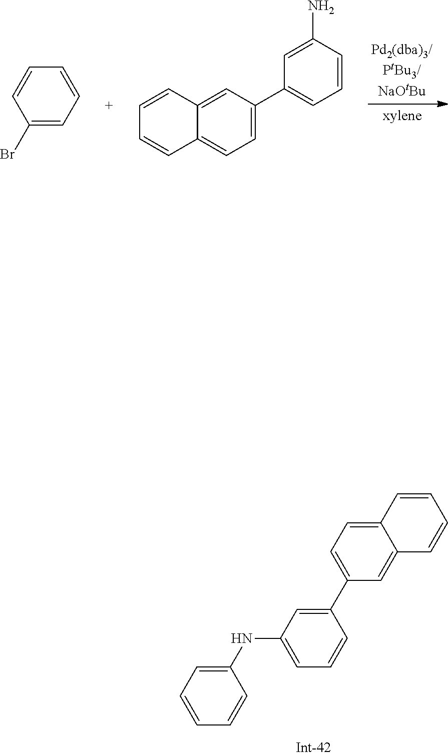 [SMILES: Brc1ccccc1 ; Nc1cccc(-c2ccc3ccccc3c2)c1 ; c1ccc(Nc2cccc(-c3ccc4ccccc4c3)c2)cc1]

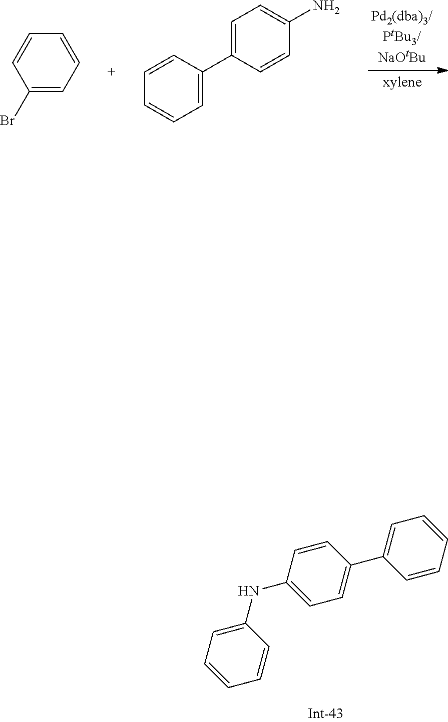 [SMILES: Brc1ccccc1 ; Nc1ccc(-c2ccccc2)cc1 ; c1ccc(Nc2ccc(-c3ccccc3)cc2)cc1]

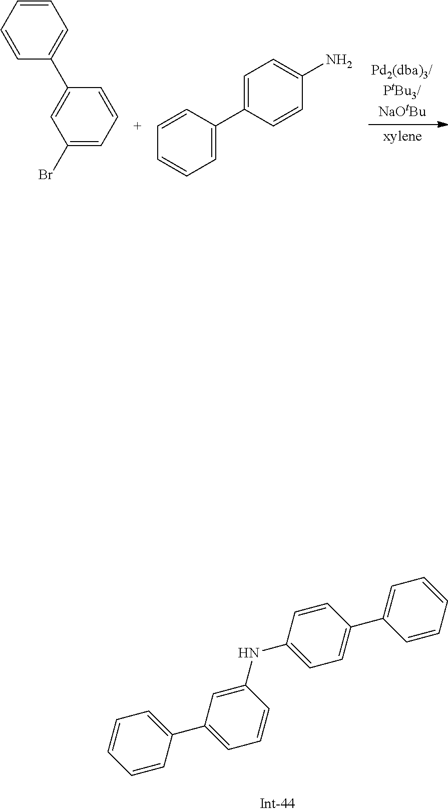

Int-40 to Int-44 were synthesized in the same manner as in the 5th step of Synthesis Example 1.

### Synthesis Examples 4 to 17

Each compound was synthesized in the same manner as in Synthesis Example 1 except that Int A shown in Table 1 instead of Int-6 and Int B shown in Table 1 instead of Int-7 were used in the 5th step of Synthesis Example 1.

TABLE 1

| Synthesis | Int | Int | Final | Amount | |
|---|---|---|---|---|---|
| Example | A | B | product | (yield) | Physical property data of final product |
| Synthesis | Int- | Int- | Compound | 3.39 g | calcd. C44H27NOS: C, 85.55; H, 4.41; |
| Example 4 | 12 | 7 | 1-7 | (77%) | N, 2.27; O, 2.59; S, 5.19 found: C, 85.55; |
| | | | | | H, 4.41; N, 2.27; O, 2.59; S, 5.19 |
| Synthesis | Int- | Int- | Compound | 3.5 g | calcd. C44H27NOS: C, 85.55; H, 4.41; |
| Example 5 | 17 | 7 | 1-67 | (73%) | N, 2.27; O, 2.59; S, 5.19 found: C, 85.55; |
| | | | | | H, 4.41; N, 2.27; O, 2.59; S, 5.19 |
| Synthesis | Int- | Int- | Compound | 5.5 g | calcd. C44H27NOS: C, 85.55; H, 4.41; |
| Example 6 | 22 | 7 | 1-194 | (73%) | N, 2.27; O, 2.59; S, 5.19 found: C, 85.56; |
| | | | | | H, 4.41; N, 2.27; O, 2.58; S, 5.19 |
| Synthesis | Int- | Int- | Compound | 7.8 g | calcd. C44H27NOS: C, 85.55; H, 4.41; |
| Example 7 | 27 | 7 | 1-259 | (75%) | N, 2.27; O, 2.59; S, 5.19 found: C, 85.55; |
| | | | | | H, 4.41; N, 2.27; O, 2.59; S, 5.19 |
| Synthesis | Int- | Int- | Compound | 4.6 g | calcd. C44H27NOS: C, 85.55; H, 4.41; |
| Example 8 | 31 | 7 | 1-291 | (73%) | N, 2.27; O, 2.59; S, 5.19 found: C, 85.55; |
| | | | | | H, 4.41; N, 2.27; O, 2.59; S, 5.19 |
| Synthesis | Int- | Int- | Compound | 5.9 g | calcd. C44H27NOS: C, 85.55; H, 4.41; |
| Example 9 | 35 | 7 | 1-322 | (79%) | N, 2.27; O, 2.59; S, 5.19 found: C, 85.54; |
| | | | | | H, 4.41; N, 2.28; O, 2.59; S, 5.19 |
| Synthesis | Int- | Int- | Compound | 6.5 g | calcd. C44H27NOS: C, 85.55; H, 4.41; |
| Example 10 | 39 | 7 | 1-354 | (70%) | N, 2.27; O, 2.59; S, 5.19 found: C, 85.56; |
| | | | | | H, 4.41; N, 2.27; O, 2.59; S, 5.18 |
| Synthesis | Int- | Int- | Compound | 6.37 g | calcd. C50H29NO2S: C, 84.84; H, 4.13; |
| Example 11 | 17 | 40 | 1-106 | (70%) | N, 1.98; O, 4.52; S, 4.53 found: C, 84.84; |
| | | | | | H, 4.13; N, 1.98; O, 4.52; S, 4.53 |
| Synthesis | Int- | Int- | Compound | 5.09 g | calcd. C52H35NOSSi: C, 83.28; H, 4.70; |
| Example 12 | 17 | 41 | 1-107 | (65%) | N, 1.87; O, 2.13; S, 4.27; Si, 3.74 found: |
| | | | | | C, 83.28; H, 4.70; N, 1.87; O, 2.13; S, |
| | | | | | 4.27; Si, 3.74 |
| Synthesis | Int- | Int- | Compound | 5.92 g | calcd. C44H27NOS: C, 85.55; H, 4.41; |
| Example 13 | 17 | 42 | 1-111 | (73%) | N, 2.27; O, 2.59; S, 5.19 found: C, 85.55; |
| | | | | | H, 4.41; N, 2.27; O, 2.59; S, 5.19 |
| Synthesis | Int- | Int- | Compound | 4.38 g | calcd. C40H25NOS: C, 84.63; H, 4.44; |
| Example 14 | 17 | 43 | 1-122 | (67%) | N, 2.47; O, 2.82; S, 5.65 found: C, 84.64; |
| | | | | | H, 4.44; N, 2.46; O, 2.82; S, 5.65 |
| Synthesis | Int- | Int- | Compound | 5.92 g | calcd. C46H29NOS: C, 85.82; H, 4.54; |
| Example 15 | 17 | 44 | 1-125 | (73%) | N, 2.18; O, 2.49; S, 4.98 found: C, 85.82; |
| | | | | | H, 4.54; N, 2.18; O, 2.49; S, 4.98 |
| Synthesis | Int- | Int- | Compound | 4.30 g | calcd. C40H25NOS: C, 84.63; H, 4.44; |
| Example 16 | 6 | 43 | 1-171 | (78%) | N, 2.47; O, 2.82; S, 5.65 found: C, 84.64; |
| | | | | | H, 4.44; N, 2.46; O, 2.82; S, 5.65 |
| Synthesis | Int- | Int- | Compound | 8.38 g | calcd. C40H25NOS: C, 84.63; H, 4.44; |
| Example 17 | 31 | 43 | 1-304 | (64%) | N, 2.47; O, 2.82; S, 5.65 found: C, 84.63; |
| | | | | | H, 4.44; N, 2.47; O, 2.82; S, 5.65 |

### Synthesis Example 18: Synthesis of Compound A-3

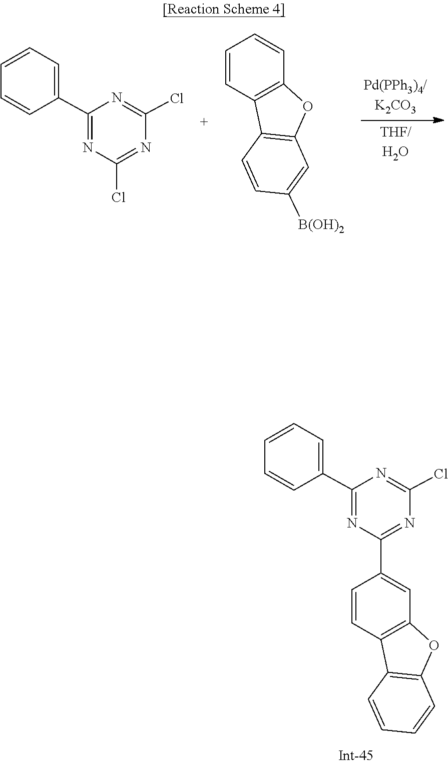 [SMILES: Clc1nc(Cl)nc(-c2ccccc2)n1 ; OB(O)c1ccc2c(c1)oc1ccccc12 ; Clc1nc(-c2ccccc2)nc(-c2ccc3c(c2)oc2ccccc23)n1]

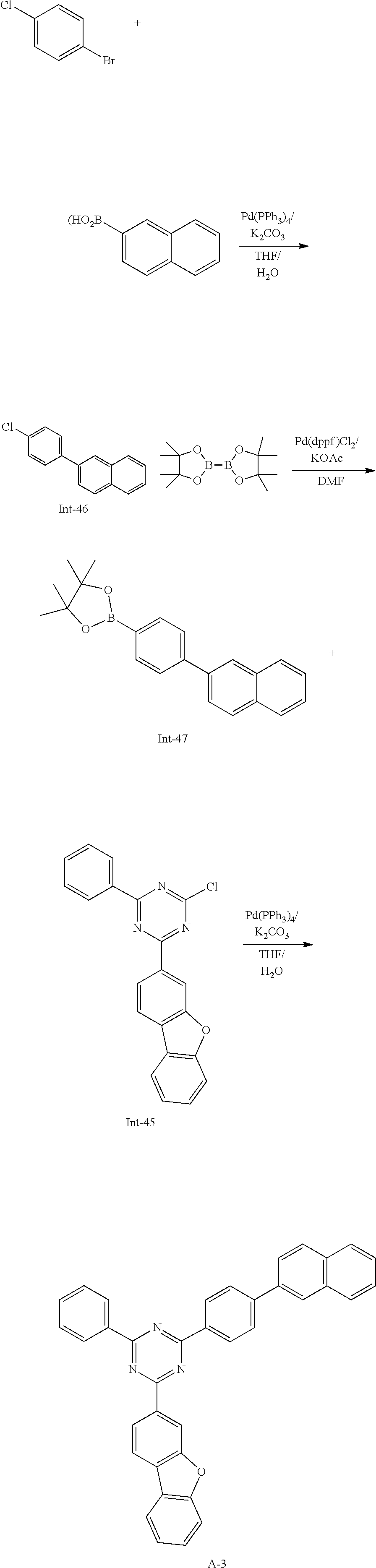

### 1st Step: Synthesis of Int-45

In a round-bottomed flask, 22.6 g (100 mmol) of 2,4-dichloro-6-phenyl-1,3,5-triazine was added to 200 mL of tetrahydrofuran and 100 mL of distilled water, and 0.9 equivalent of dibenzofuran-3-boronic acid (CAS No.: 395087-89-5), 0.03 equivalent of tetrakistriphenylphosphine palladium, and 2 equivalent of potassium carbonate were added thereto and then, heated and refluxed under a nitrogen atmosphere. After 6 hours, the reaction solution was cooled, and an organic layer therefrom after removing an aqueous layer was dried under a reduced pressure. A solid obtained therefrom was washed with water and hexane and recrystallized with 200 mL of toluene to obtain 21.4 g (a yield: 60%) of Int-45.

### 2nd Step: Synthesis of Int-46

In a round-bottomed flask, 50.0 g (261.16 mmol) of 1-bromo-4-chloro-benzene, 44.9 g (261.16 mmol) of 2-naphthalene boronic acid, 9.1 g (7.83 mmol) of tetrakistriphenylphosphine palladium, and 71.2 g (522.33 mmol) of potassium carbonate were dissolved in 1000 mL of tetrahydrofuran and 500 mL of distilled water and then, heated and refluxed under a nitrogen atmosphere. After 6 hours, the reaction solution was cooled, and an organic layer therefrom after removing an aqueous layer was dried under a reduced pressure. A solid obtained therefrom was washed with water and hexane and then, recrystallized with 200 mL of toluene to obtain 55.0 g (a yield: 88%) of Int-46.

### 3rd Step: Synthesis of Int-47

In a round-bottomed flask, 100.0 g (418.92 mmol) of the synthesized Int-46 was added to 1000 mL of DMF, and 17.1 g (20.95 mmol) of dichlorodiphenylphosphinoferrocene palladium, 127.7 g (502.70 mmol) of bispinacolato diboron, and 123.3 g (1256.76 mmol) of potassium acetate were added thereto and then, heated and refluxed under a nitrogen atmosphere for 12 hours. The reaction solution was cooled and then, added dropwise to 2 L of water to catch a solid. The obtained solid was dissolved in boiling toluene and then, filtered through silica gel, and a filter therefrom was concentrated. The concentrated solid was stirred with a small amount of hexane and filtered to obtain 28.5 g (a yield: 70%) of Int-47.

### 4th Step: Synthesis of Compound A-3

In a round-bottomed flask, 10.0 g (27.95 mmol) of Int-47, 11.1 g (33.54 mmol) of Int-45, 1.0 g (0.84 mmol) of tetrakistriphenylphosphine palladium, and 7.7 g (55.90 mmol) of potassium carbonate were dissolved in 150 mL of tetrahydrofuran and 75 mL of distilled water and then, heated and refluxed under a nitrogen atmosphere. After 12 hours, the reaction solution was cooled, after removing an aqueous layer, an organic layer therefrom was dried under a reduced pressure. The obtained solid was washed with water and methanol and then, recrystallized with 200 mL of toluene to obtain 13.4 g (a yield: 91%) of compound A-3.

calcd. C37H23N3O: C, 84.55; H, 4.41; N, 7.99; O, 3.04; found: C, 84.55; H, 4.41; N, 8.00; O, 3.03

### Synthesis Example 19: Synthesis of Compound A-17

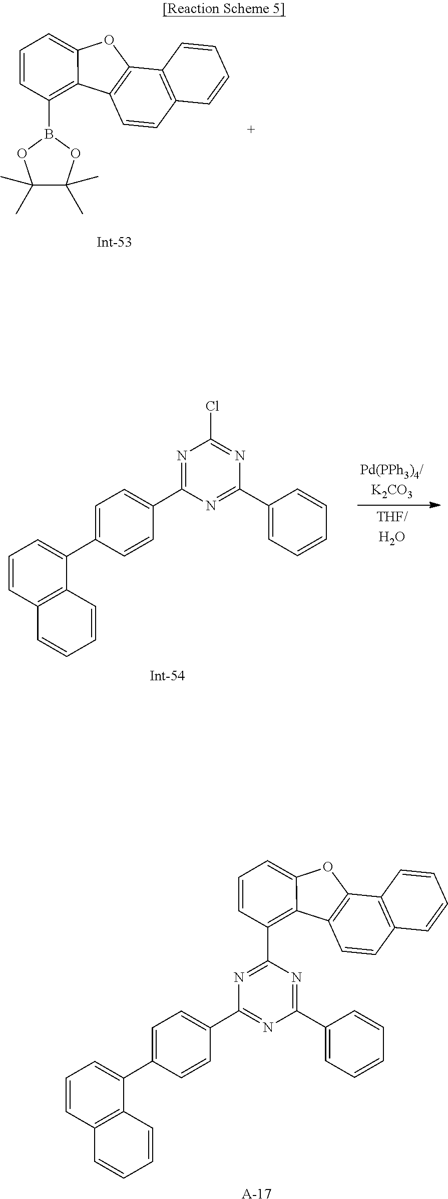 [SMILES: CC1(C)OB(c2cccc3oc4c5ccccc5ccc4c23)OC1(C)C ; Clc1nc(-c2ccccc2)nc(-c2ccc(-c3cccc4ccccc34)cc2)n1 ; c1ccc(-c2nc(-c3ccc(-c4cccc5ccccc45)cc3)nc(-c3cccc4oc5c6ccccc6ccc5c34)n2)cc1 ; "A-17"]

Compound A-17 was synthesized in the same manner as in the 4th step of Synthesis Example 18 except that Int-53 and Int-54 were respectively used by 1.0 equivalent.

calcd. C41H25N₃O: C, 85.54; H, 4.38; N, 7.30; O, 2.78; found: C, 85.53; H, 4.38; N, 7.30; O, 2.77

### Synthesis Example 20: Synthesis of Compound A-37

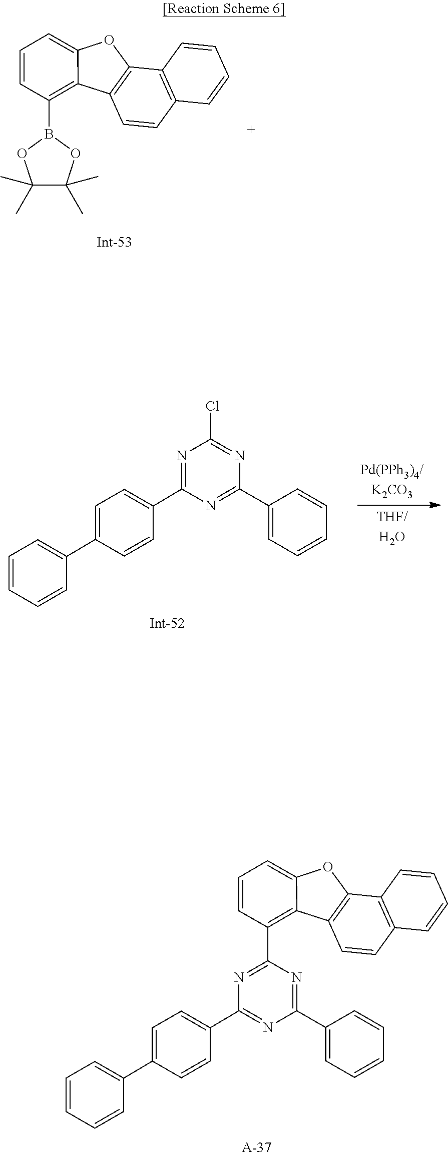 [SMILES: CC1(C)OB(c2cccc3oc4c5ccccc5ccc4c23)OC1(C)C ; Clc1nc(-c2ccccc2)nc(-c2ccc(-c3ccccc3)cc2)n1 ; c1ccc(-c2ccc(-c3nc(-c4ccccc4)nc(-c4cccc5oc6c7ccccc7ccc6c45)n3)cc2)cc1 ; "A-37"]

Compound A-37 was synthesized in the same manner as in the 4th step of Synthesis Example 18 except that Int-53 and Int-52 were respectively used by 1.0 equivalent.

calcd. C37H23N₃O: C, 84.55; H, 4.41; N, 7.99; O, 3.04; found: C, 84.57; H, 4.40; N, 7.99; O, 3.03

### Synthesis Examples 21 and 22

Each compound was synthesized in the same manner as in the 4th step of Synthesis Example 18 except that Int C of Table 2 instead of Int-47 of Synthesis Example 18 and Int D shown in Table 2 instead of Int-45 were used.

TABLE 2

| Synthesis | | | Final | Amount | Physical property data of |
|---|---|---|---|---|---|
| Example | Int C | Int D | product | (yield) | final product |
| Synthesis Example 21 | 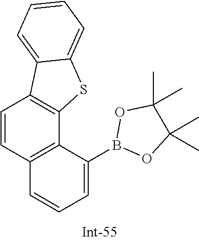 | Int-54 | Compound A-24 | 8.33 g, 74% | calcd. C41H25N3S: C, 83.22; H, 4.26; N, 7.10; S, 5.42 found: C, 83.22; H, 4.26; N, 7.10; S, 5.42 |
| Synthesis Example 22 | 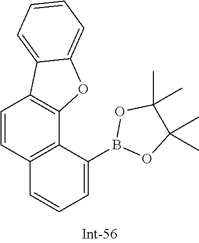 | 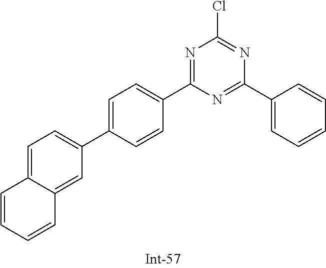 | Compound A-35 | 7.67 g, 71% | calcd. C41H25N3O: C, 85.54; H, 4.38; N, 7.30; O, 2.78 found: C, 85.55; H, 4.38; N, 7.29; O, 2.7 |

### Comparative Synthesis Example 1: Synthesis of Compound C-1

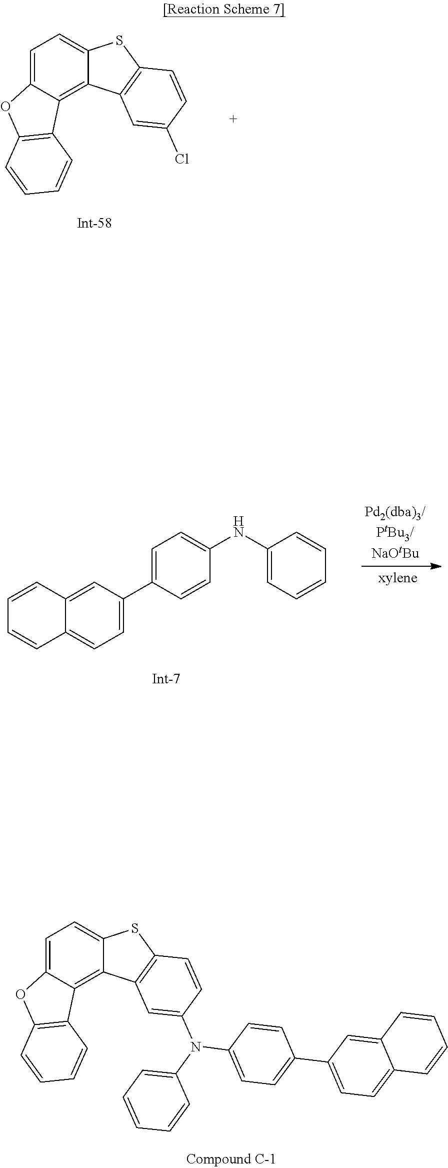 [SMILES: Clc1ccc2sc3ccc4oc5ccccc5c4c3c2c1 ; c1ccc(Nc2ccc(-c3ccc4ccccc4c3)cc2)cc1 ; c1ccc(N(c2ccc(-c3ccc4ccccc4c3)cc2)c2ccc3sc4ccc5oc6ccccc6c5c4c3c2)cc1]

Compound C-1 was synthesized in the same manner as in the 5th step of Synthesis Example 1 except that Int-58 was used instead of Int-6.

calcd. C40H25NOS: C, 84.63; H, 4.44; N, 2.47; O, 2.82; S, 5.65 found: C, 84.63; H, 4.44; N, 2.47; O, 2.82; S, 5.65

### (Manufacture of Organic Light Emitting Diode)

### Example 1

The glass substrate coated with ITO (Indium tin oxide) at a thickness of 1,500 Å was washed with distilled water. After washing with the distilled water, the glass substrate was washed with a solvent such as isopropyl alcohol, acetone, methanol, and the like ultrasonically and dried and then, moved to a plasma cleaner, cleaned by using oxygen plasma for 10 minutes, and moved to a vacuum depositor. This prepared ITO transparent electrode was used as an anode, Compound A doped with 3% NDP-9 (Novaled GmbH) was vacuum-deposited on the ITO substrate to form a 100 Å-thick hole injection layer, and Compound A is deposited on the hole injection layer to a thickness of 1300 Å to form a hole transport layer. Compound B was deposited on the hole transport layer to a thickness of 700 Å to form a hole transport auxiliary layer. Compound 1-130 obtained in Synthesis Example 1 was used as a host on the hole transport layer, and 2 wt % of [Ir(piq)₂acac] was doped as a dopant to form a 400 Å-thick light emitting layer by vacuum deposition. Subsequently, Compound C was deposited on the emitting layer to a thickness of 50 Å to form an electron transport auxiliary layer, and Compound D and LiQ were simultaneously vacuum-deposited at a weight ratio of 1:1 to form an electron transport layer with a thickness of 300 Å. An organic light emitting diode was manufactured by sequentially vacuum depositing 15 Å of LiQ and 1200 Å of Al on the electron transport layer to form a cathode.

ITO/Compound A (3% NDP-9 doping, 100 Å)/Compound A (1300 Å)/Compound B (700 Å)/EML [Host (Compound 1-130, 98 wt %), [Ir(piq)₂acac] (2 wt) %)] (400 Å)/Compound C (50 Å)/Compound D: Liq (300 Å)/LiQ (15 Å)/Al (1200 Å).

Compound A: N-(biphenyl-4-yl)-9,9-dimethyl-N-(4-(9-phenyl-9H-carbazol-3-yl)phenyl)-9H-fluoren-2-amine

Compound B: N,N-di([1,1′-biphenyl]-4-yl)-7,7-dimethyl-7H-fluoreno[4,3-b]benzofuran-10-amine

Compound C: 2-(3-(3-(9,9-dimethyl-9H-fluoren-2-yl)phenyl)phenyl)-4,6-diphenyl-1,3,5-triazine

Compound D: 8-(4-(4,6-di(naphthalen-2-yl)-1,3,5-triazin-2-yl)phenyl)quinoline Examples 2 to 15, and Comparative Examples 1

Each organic light emitting diode was manufactured in the same manner as Example 1, except that the host was changed as shown in Table 3.

### Example 16

The glass substrate coated with ITO (Indium tin oxide) at a thickness of 1,500 Å was washed with distilled water. After washing with the distilled water, the glass substrate was washed with a solvent such as isopropyl alcohol, acetone, methanol, and the like ultrasonically and dried and then, moved to a plasma cleaner, cleaned by using oxygen plasma for 10 minutes, and moved to a vacuum depositor. This prepared ITO transparent electrode was used as an anode, Compound A doped with 3% NDP-9 (Novaled GmbH) was vacuum-deposited on the ITO substrate to form a 100 Å-thick hole injection layer, and Compound A is deposited on the hole injection layer to a thickness of 1300 Å to form a hole transport layer. Compound B was deposited on the hole transport layer to a thickness of 700 Å to form a hole transport auxiliary layer. On the hole transport auxiliary layer, Compound 1-130 obtained in Synthesis Example 1 and Compound A-17 obtained in Synthesis Example 19 were used simultaneously as a host, and 2 wt % of [Ir(piq)₂acac] was doped as a dopant to form a 400 Å-thick light emitting layer by vacuum deposition. Herein, Compound 1-130 and Compound A-17 were used at a weight ratio of 5:5. Subsequently, Compound C was deposited on the emitting layer to a thickness of 50 Å to form an electron transport auxiliary layer, and Compound D and LiQ were simultaneously vacuum-deposited at a weight ratio of 1:1 to form an electron transport layer with a thickness of 300 Å. An organic light emitting diode was manufactured by sequentially vacuum depositing 15 Å of LiQ and 1200 Å of Al on the electron transport layer to form a cathode.

ITO/Compound A (3% NDP-9 doping, 100 Å)/Compound A (1300 Å)/Compound B (700 Å)/EML [Host (Compound 1-130: Compound A-17=5:5): [Ir(piq)₂acac]=98 wt %: 2 wt %] (400 Å)/Compound C (50 Å)/Compound D: Liq (300 Å)/LiQ (15 Å)/Al (1200 Å). Examples 17 to 31, and Comparative Example 2

Each organic light emitting diode was manufactured in the same manner as Example 16, except that the host was changed as shown in Table 4.

### Evaluation

The luminous efficiency and life-span characteristics of the organic light emitting diodes according to Examples 1 to 31 and Comparative Examples 1 and 2 were evaluated. The specific measurement method is as follows, and the results are as shown in Tables 3 and 4.

### (1) Measurement of Current Density Change Depending on Voltage Change

The obtained organic light emitting diodes were measured regarding a current value flowing in the unit device, while increasing the voltage from 0 V to 10 V using a current-voltage meter (Keithley 2400), and the measured current value was divided by area to provide the results.

### (2) Measurement of Luminance Change Depending on Voltage Change

Luminance was measured by using a luminance meter (Minolta Cs-1000A), while the voltage of the organic light emitting diodes was increased from 0 V to 10 V.

### (3) Measurement of Luminous Efficiency

Luminous efficiency (cd/A) at the same current density (10 mA/cm²) were calculated by using the luminance and current density from (1) and (2) above and voltage.

Relative values based on the luminous efficiency of Comparative Example 1 were calculated and shown in Table 3.

Relative values based on the luminous efficiency of Comparative Example 2 were calculated and shown in Table 4.

### (4) Measurement of Life-Span

T95 life-spans of the diodes according to Examples 1 to 31 and Comparative Examples 1 and 2 were measured as a time when their luminance decreased down to 95% relative to the initial luminance (cd/m2) after emitting light with 6,000 cd/m² as the initial luminance (cd/m²) and measuring their luminance decreases depending on a time with a Polanonix life-span measurement system.

Relative values based on the T95 life-span of Comparative Example 1 were calculated and shown in Table 3.

Relative values based on the T95 life-span of Comparative Example 2 were calculated and shown in Table 4.

TABLE 3

| | Host | Efficiency (%) | T95 life-span (%) |
|---|---|---|---|
| Example 1 | 1-130 | 117% | 114% |
| Example 2 | 1-7 | 108% | 109% |
| Example 3 | 1-67 | 115% | 119% |
| Example 4 | 1-106 | 117% | 116% |
| Example 5 | 1-107 | 118% | 115% |
| Example 6 | 1-111 | 118% | 114% |
| Example 7 | 1-122 | 120% | 119% |
| Example 8 | 1-125 | 117% | 116% |
| Example 9 | 1-171 | 110% | 113% |
| Example 10 | 1-194 | 110% | 116% |
| Example 11 | 1-259 | 110% | 112% |
| Example 12 | 1-291 | 114% | 118% |
| Example 13 | 1-304 | 117% | 118% |
| Example 14 | 1-322 | 112% | 115% |
| Example 15 | 1-354 | 110% | 114% |
| Comparative Example 1 | C-1 | 100% | 100% |

TABLE 4

| | Host | | Efficiency | T95 life- |
|---|---|---|---|---|
| | First host | Second host | (%) | span (%) |
| Example 16 | 1-67 | A-17 | 132% | 125% |
| Example 17 | 1-106 | | 119% | 121% |
| Example 18 | 1-111 | | 130% | 123% |
| Example 19 | 1-122 | | 121% | 116% |
| Example 20 | 1-130 | | 120% | 119% |
| Example 21 | 1-291 | | 128% | 126% |
| Example 22 | 1-322 | | 123% | 119% |
| Example 23 | 1-67 | A-24 | 130% | 123% |
| Example 24 | 1-111 | | 131% | 124% |
| Example 25 | 1-291 | | 125% | 124% |
| Example 26 | 1-67 | A-35 | 133% | 128% |
| Example 27 | 1-111 | | 132% | 126% |
| Example 28 | 1-291 | | 129% | 126% |
| Example 29 | 1-67 | A-37 | 135% | 127% |
| Example 30 | 1-111 | | 132% | 124% |
| Example 31 | 1-291 | | 131% | 126% |
| Comparative | C-1 | A-17 | 100% | 100% |
| Example 2 | | | | |

Referring to Table 3, the efficiency and life-span of the compounds according to the present invention were improved in a single host compared to the comparative compounds. In particular, referring to Table 4, when combined with a second host, the overall efficiency and life-span were significantly improved.

While this invention has been described in connection with what is presently considered to be practical example embodiments, it is to be understood that the invention is not limited to the disclosed embodiments, but, on the contrary, is intended to cover various modifications and equivalent arrangements included within the spirit and scope of the appended claims.

1. A compound for an organic optoelectronic device, the compound being represented by Chemical Formula 1: 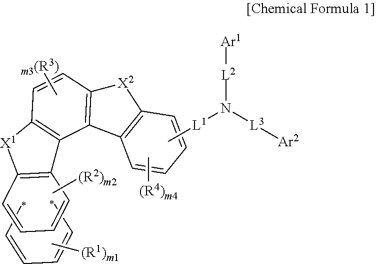
wherein, in Chemical Formula 1,
X¹ and X² are each independently O or S,
L¹ to L³ are each independently a single bond or a substituted or unsubstituted C6 to C20 arylene group,
Ar¹ and Ar² are each independently a substituted or unsubstituted C6 to C20 aryl group or a substituted or unsubstituted C2 to C30 heterocyclic group,
R¹ to R⁴ are each independently hydrogen, deuterium, a halogen, a substituted or unsubstituted C1 to C10 alkyl group or a substituted or unsubstituted C6 to C12 aryl group,
m1 is an integer of 1 to 4,
m2 and m3 are each independently 1 or 2, and
m4 is an integer of 1 to 3.

2. The compound for an organic optoelectronic device of claim 1, wherein:
Chemical Formula 1 is represented by Chemical Formula 1A, Chemical Formula 1B, or Chemical Formula 1C: 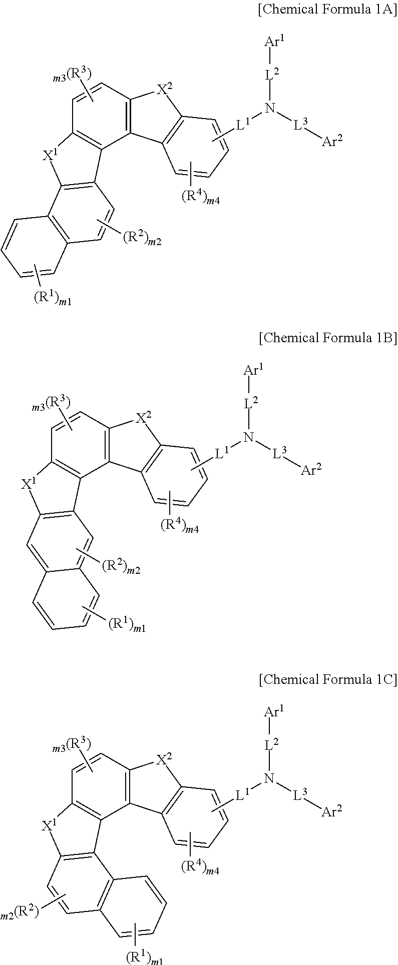
in Chemical Formula 1A, Chemical Formula 1B, Chemical Formula 1C, X¹, X², L′ to L³, Ar¹, Ar², R¹ to R⁴, and m1 to m4 are defined the same as those of Chemical Formula 1.

3. The compound for an organic optoelectronic device of claim 1, wherein;
Chemical Formula 1 is represented by any one of Chemical Formula 1A-1 to Chemical Formula 1A-4: 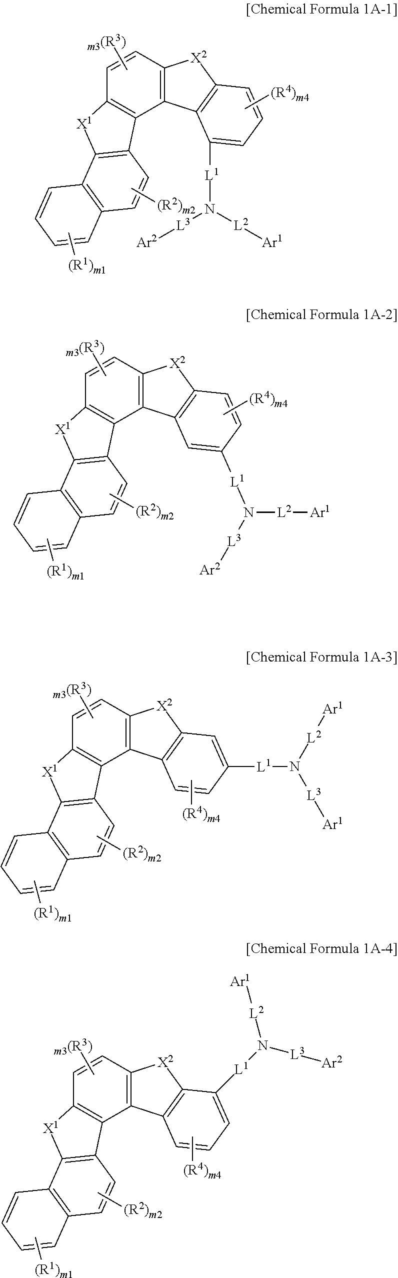
in Chemical Formula 1A-1 to Chemical Formula 1A-4, X¹, X², L¹ to L³, Ar¹, Ar², and R¹ to R⁴ are defined the same as those of Chemical Formula 1.

4. The compound for an organic optoelectronic device of claim 1, wherein Ar¹ and Ar² are each independently a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted chrysenyl group, a substituted or unsubstituted fluorenyl group, a substituted or unsubstituted triphenylene group, a substituted or unsubstituted dibenzofuranyl group, a substituted or unsubstituted dibenzothiophenyl group, a substituted or unsubstituted benzonaphthofuranyl group, a substituted or unsubstituted benzonaphthothiophenyl group, or a substituted or unsubstituted dibenzosilolyl group.

5. The compound for an organic optoelectronic device of claim 1, wherein:
Ar¹ and Ar² are each independently a group of Group I: 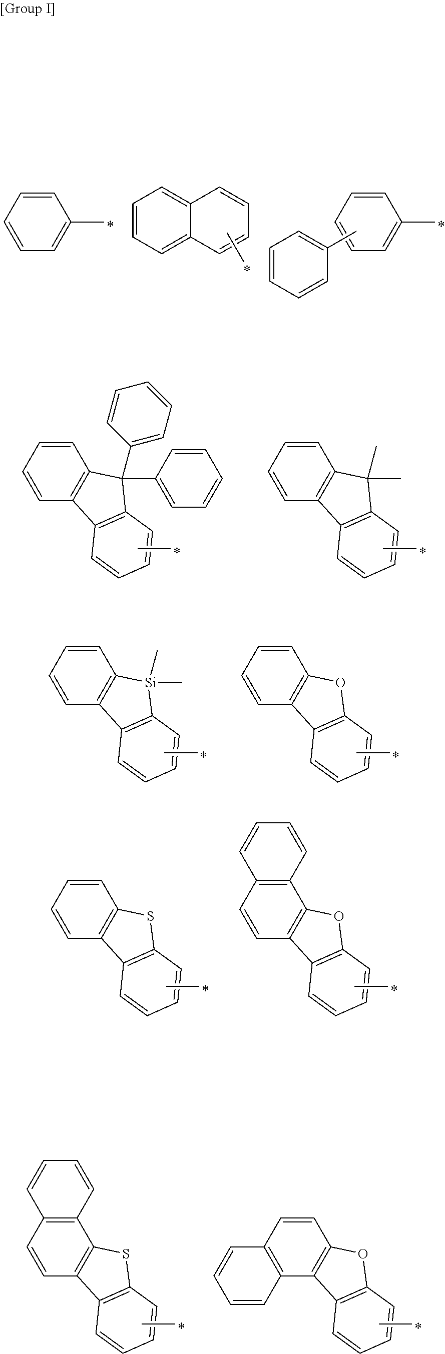 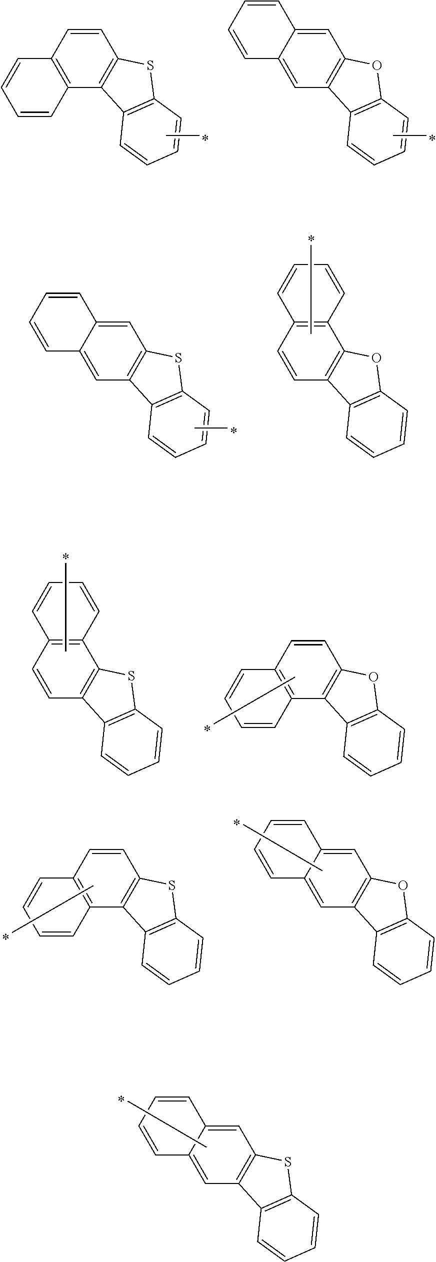
in Group I, * is a linking point.

6. The compound for an organic optoelectronic device of claim 1, wherein:
L¹ is a single bond, and
L² and L³ are each independently a single bond or a substituted or unsubstituted phenylene group.

7. The compound for an organic optoelectronic device of claim 1, wherein the compound is a compound of Group 1:

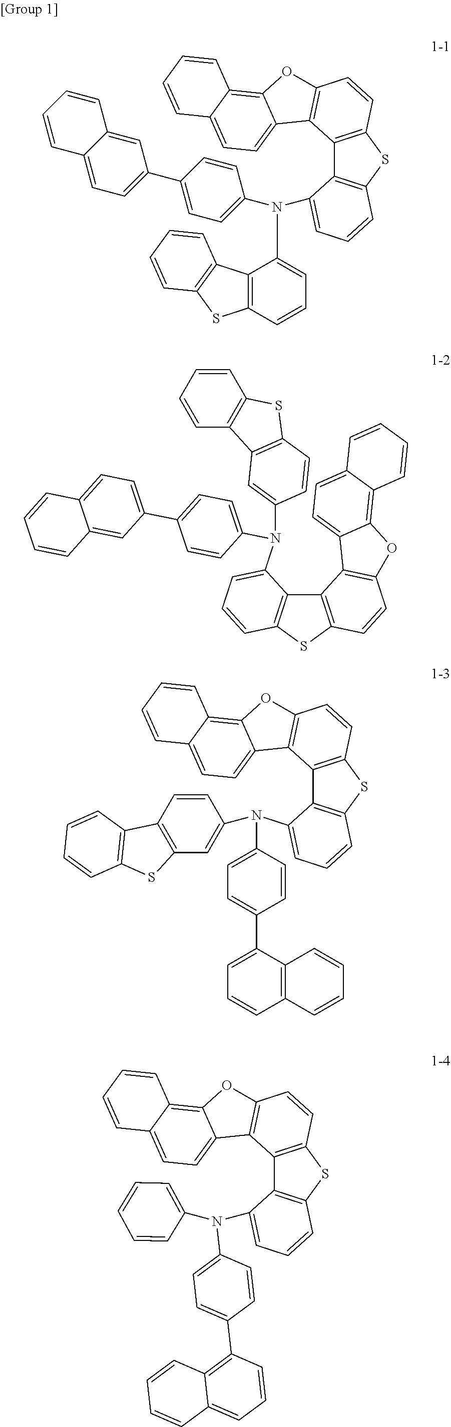 [SMILES: c1ccc2cc(-c3ccc(N(c4cccc5sc6ccccc6c45)c4cccc5sc6ccc7oc8c9ccccc9ccc8c7c6c45)cc3)ccc2c1 ; c1ccc2cc(-c3ccc(N(c4ccc5sc6ccccc6c5c4)c4cccc5sc6ccc7oc8c9ccccc9ccc8c7c6c45)cc3)ccc2c1 ; c1ccc2c(-c3ccc(N(c4ccc5c(c4)sc4ccccc45)c4cccc5sc6ccc7oc8c9ccccc9ccc8c7c6c45)cc3)cccc2c1 ; c1ccc(N(c2ccc(-c3cccc4ccccc34)cc2)c2cccc3sc4ccc5oc6c7ccccc7ccc6c5c4c23)cc1]

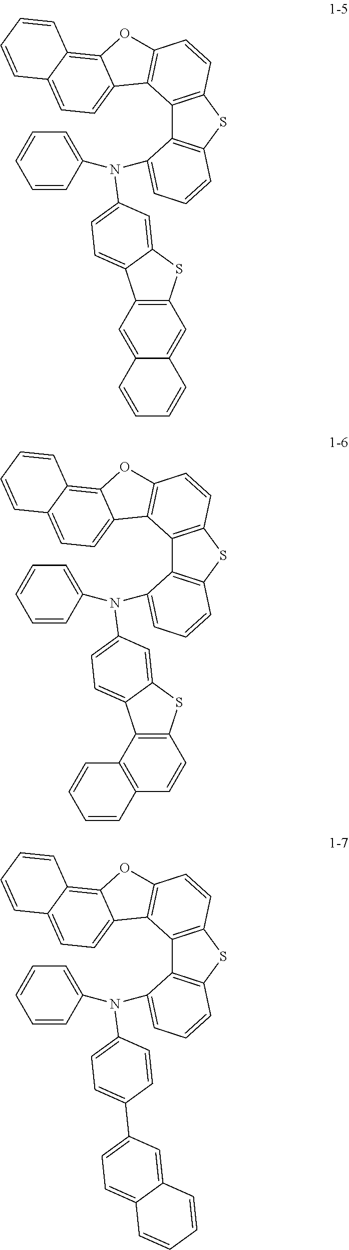 [SMILES: c1ccc(N(c2ccc3c(c2)sc2cc4ccccc4cc23)c2cccc3sc4ccc5oc6c7ccccc7ccc6c5c4c23)cc1 ; c1ccc(N(c2ccc3c(c2)sc2ccc4ccccc4c23)c2cccc3sc4ccc5oc6c7ccccc7ccc6c5c4c23)cc1 ; c1ccc(N(c2ccc(-c3ccc4ccccc4c3)cc2)c2cccc3sc4ccc5oc6c7ccccc7ccc6c5c4c23)cc1]

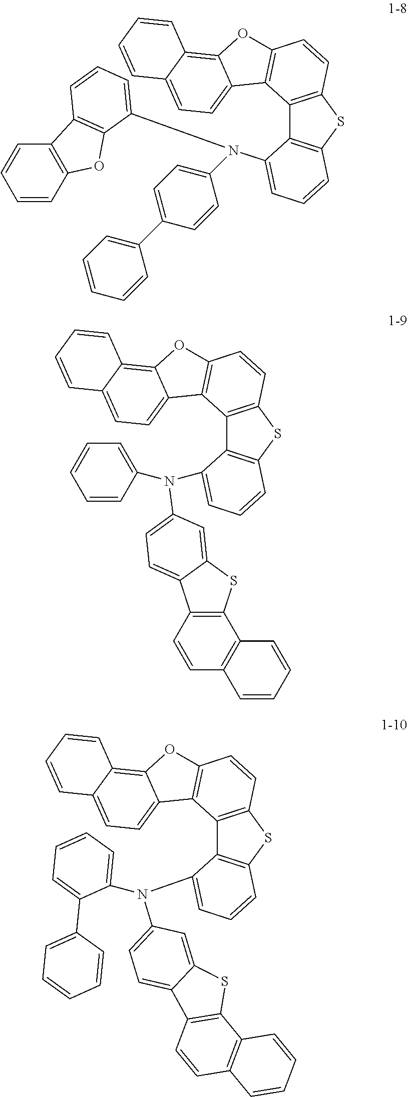

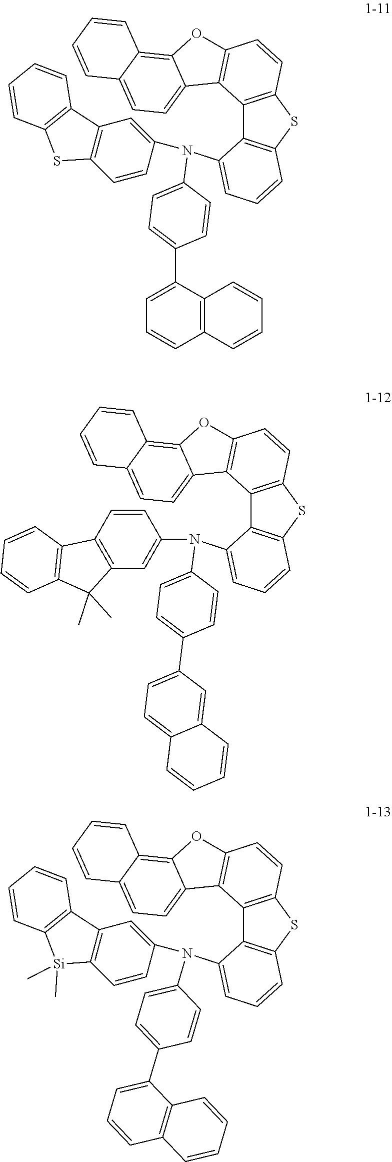

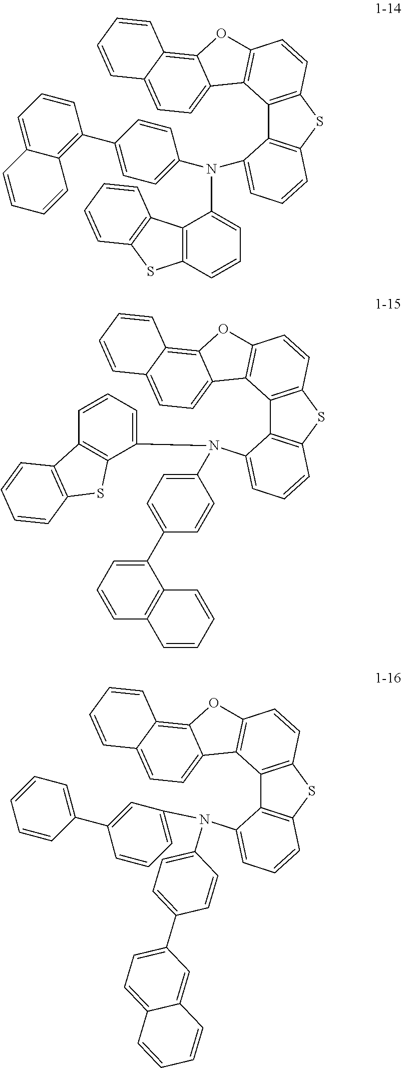

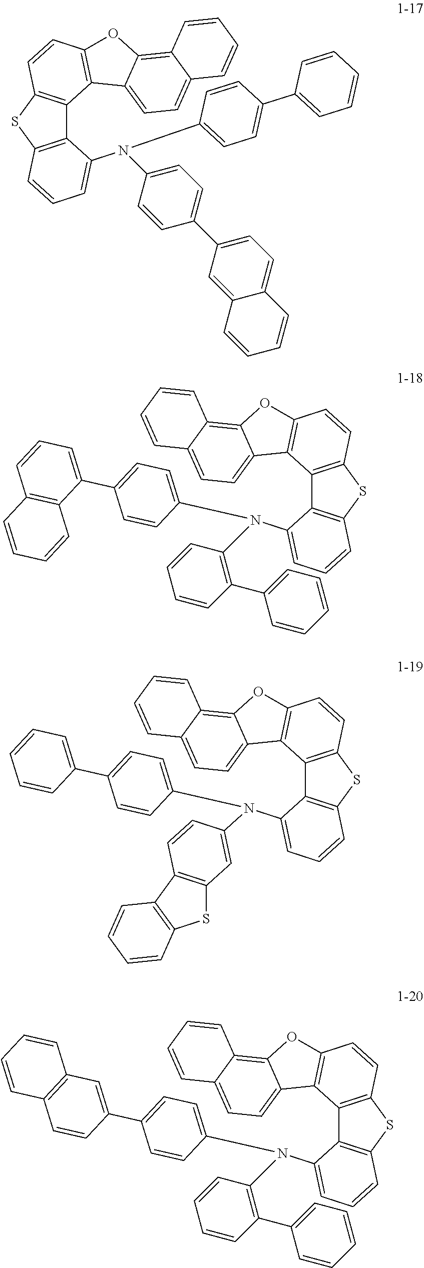 [SMILES: c1ccc(-c2ccc(N(c3ccc(-c4ccc5ccccc5c4)cc3)c3cccc4sc5ccc6oc7c8ccccc8ccc7c6c5c34)cc2)cc1 ; c1ccc(-c2ccccc2N(c2ccc(-c3cccc4ccccc34)cc2)c2cccc3sc4ccc5oc6c7ccccc7ccc6c5c4c23)cc1 ; c1ccc(-c2ccc(N(c3ccc4c(c3)sc3ccccc34)c3cccc4sc5ccc6oc7c8ccccc8ccc7c6c5c34)cc2)cc1 ; c1ccc(-c2ccccc2N(c2ccc(-c3ccc4ccccc4c3)cc2)c2cccc3sc4ccc5oc6c7ccccc7ccc6c5c4c23)cc1]

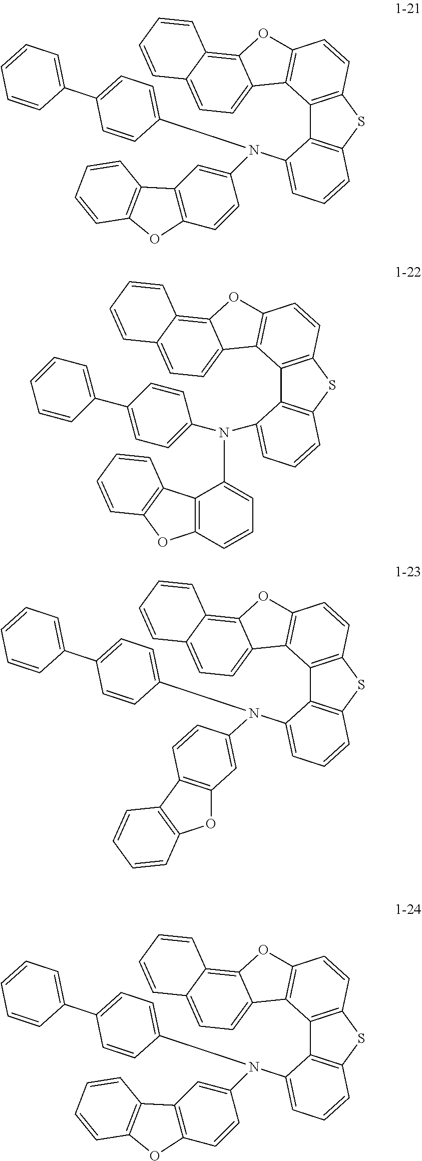

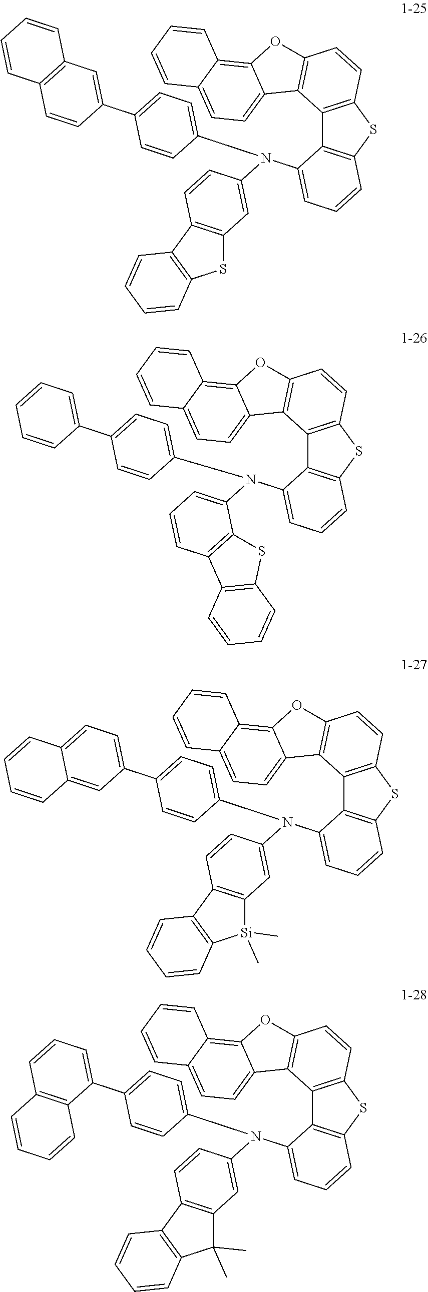

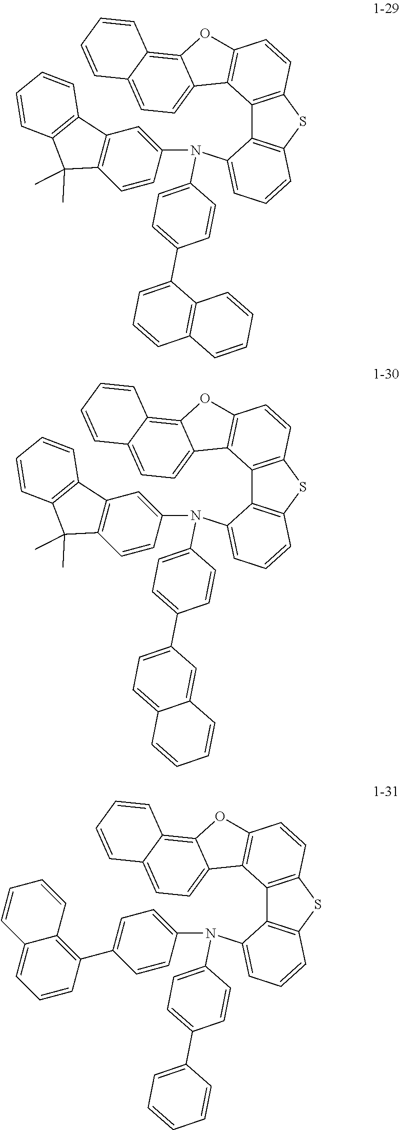

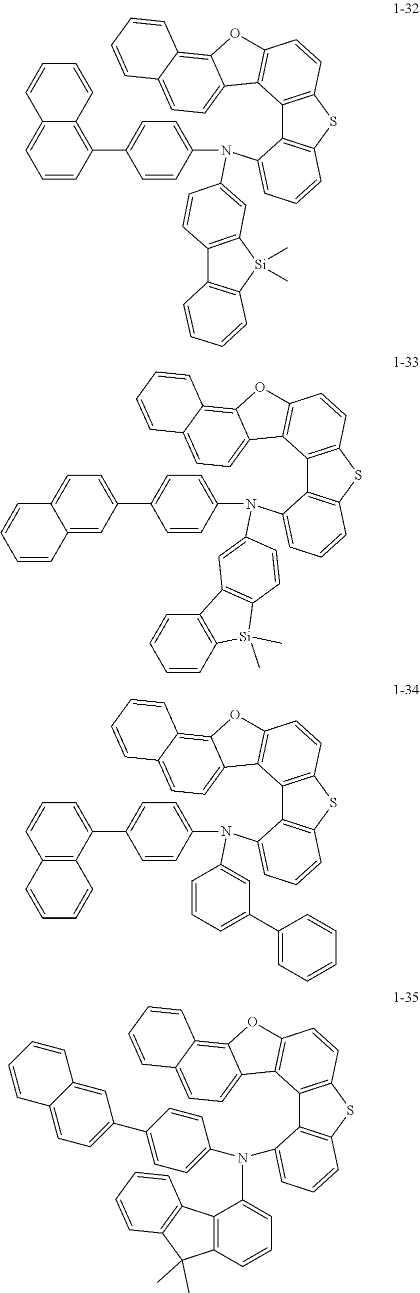

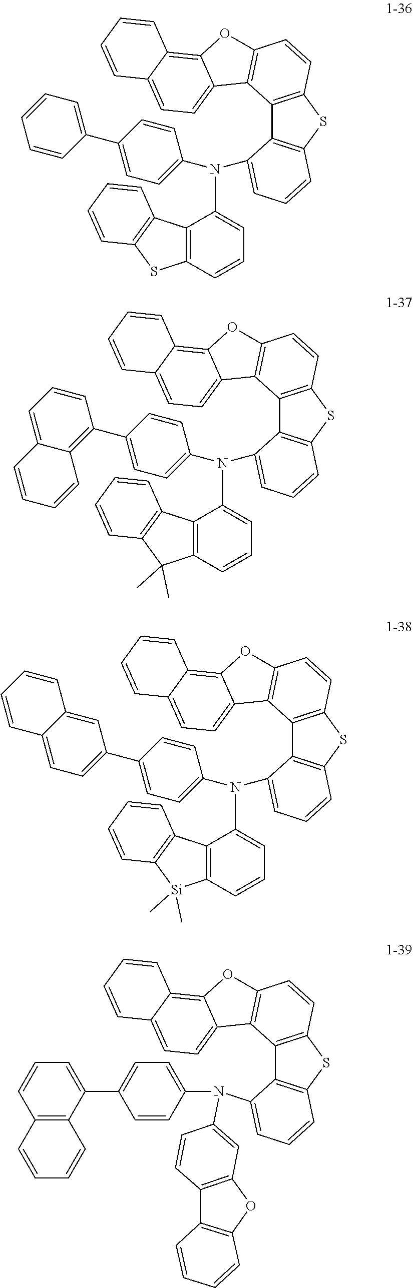

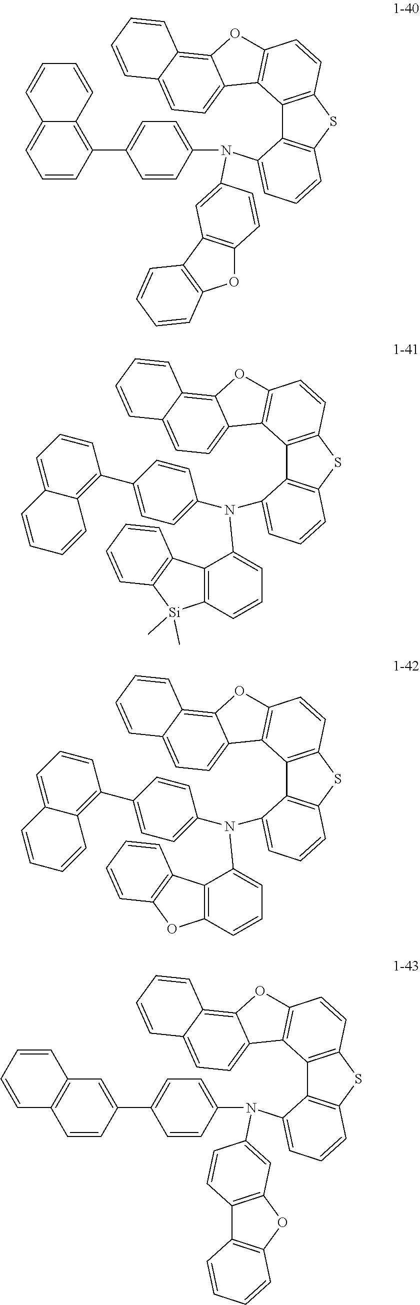

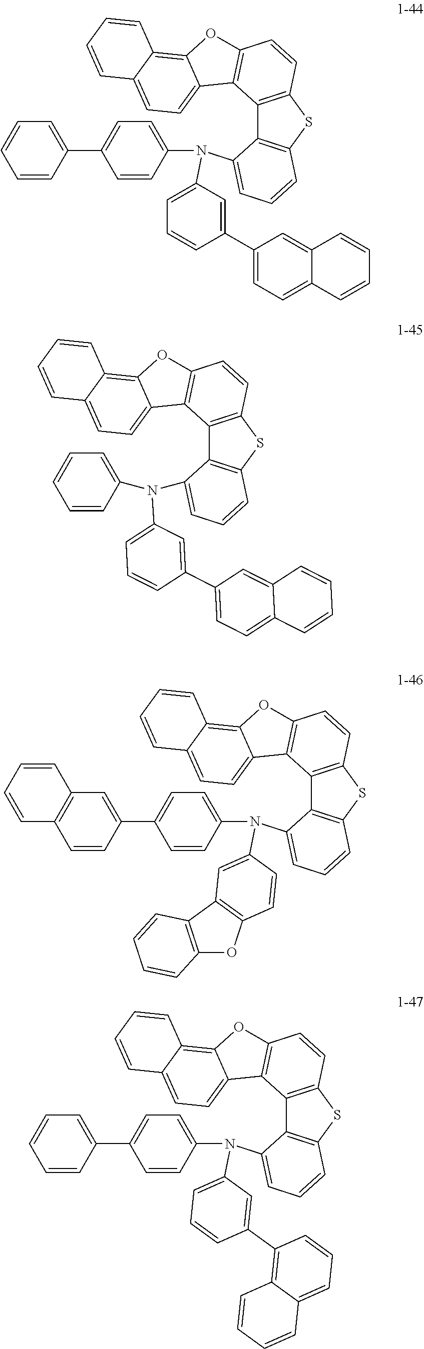

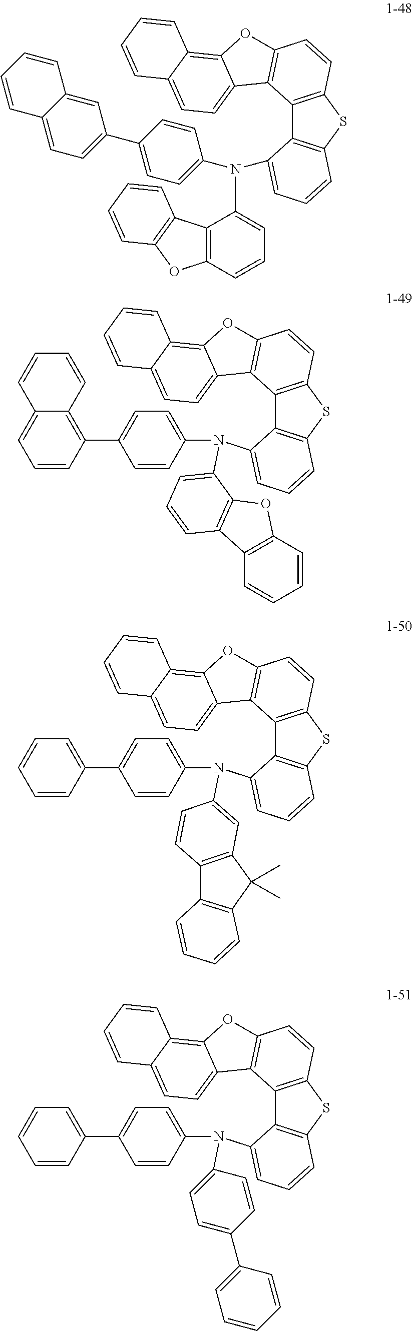

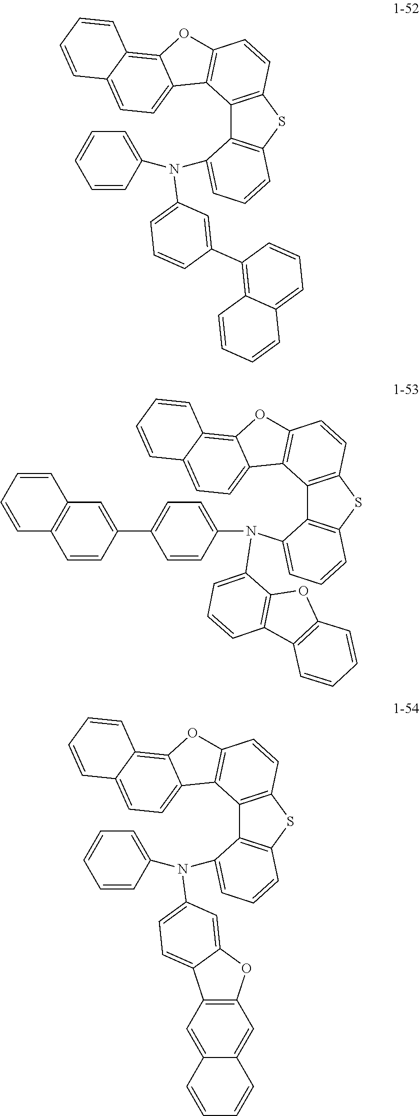 [SMILES: c1ccc(N(c2cccc(-c3cccc4ccccc34)c2)c2cccc3sc4ccc5oc6c7ccccc7ccc6c5c4c23)cc1 ; c1ccc2cc(-c3ccc(N(c4cccc5c4oc4ccccc45)c4cccc5sc6ccc7oc8c9ccccc9ccc8c7c6c45)cc3)ccc2c1 ; c1ccc(N(c2ccc3c(c2)oc2cc4ccccc4cc23)c2cccc3sc4ccc5oc6c7ccccc7ccc6c5c4c23)cc1]

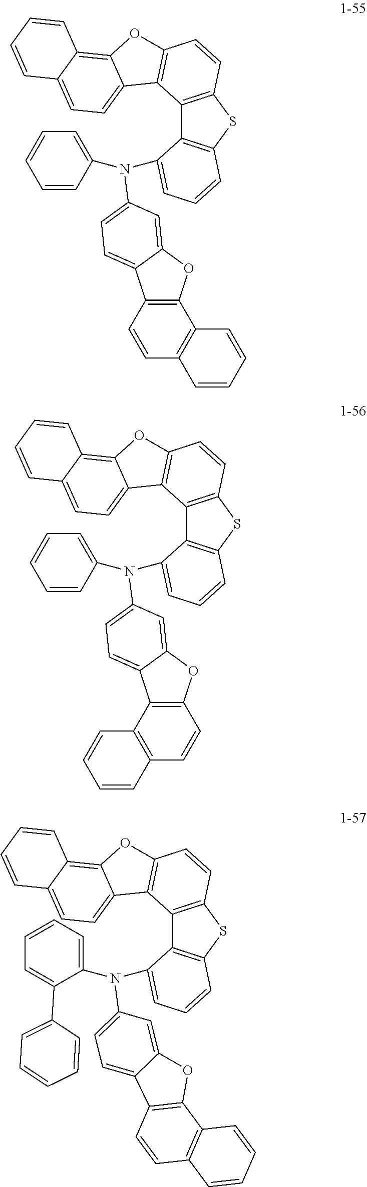 [SMILES: c1ccc(N(c2ccc3c(c2)oc2c4ccccc4ccc32)c2cccc3sc4ccc5oc6c7ccccc7ccc6c5c4c23)cc1 ; c1ccc(N(c2ccc3c(c2)oc2ccc4ccccc4c23)c2cccc3sc4ccc5oc6c7ccccc7ccc6c5c4c23)cc1 ; c1ccc(-c2ccccc2N(c2ccc3c(c2)oc2c4ccccc4ccc32)c2cccc3sc4ccc5oc6c7ccccc7ccc6c5c4c23)cc1]

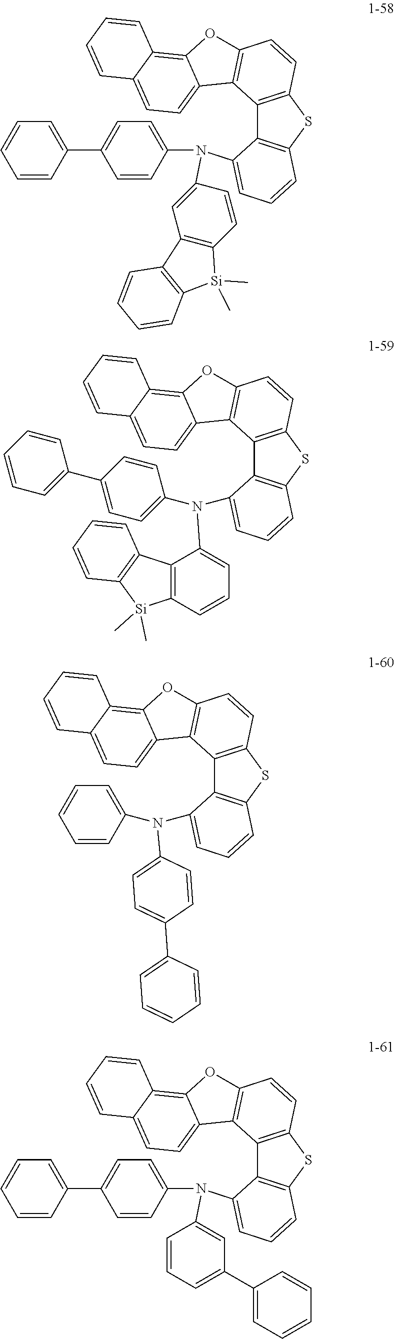

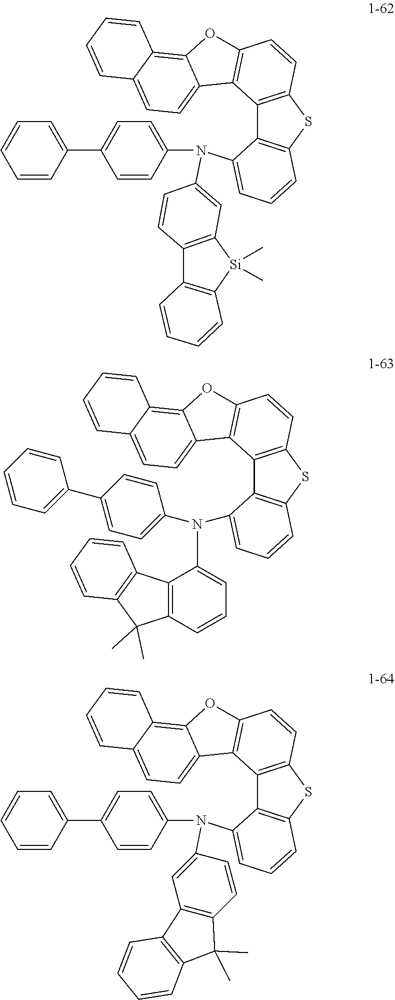

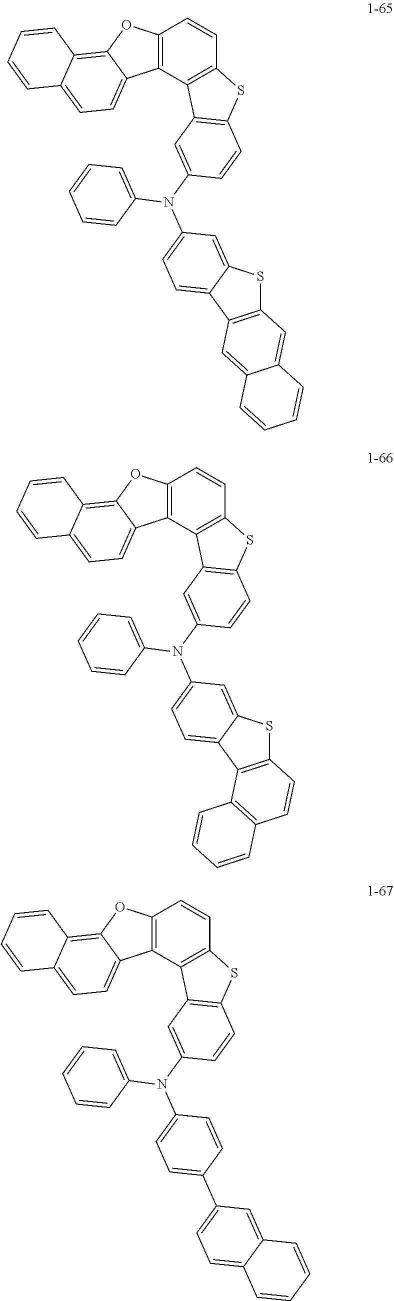 [SMILES: c1ccc(N(c2ccc3c(c2)sc2cc4ccccc4cc23)c2ccc3sc4ccc5oc6c7ccccc7ccc6c5c4c3c2)cc1 ; c1ccc(N(c2ccc3c(c2)sc2ccc4ccccc4c23)c2ccc3sc4ccc5oc6c7ccccc7ccc6c5c4c3c2)cc1 ; c1ccc(N(c2ccc(-c3ccc4ccccc4c3)cc2)c2ccc3sc4ccc5oc6c7ccccc7ccc6c5c4c3c2)cc1]

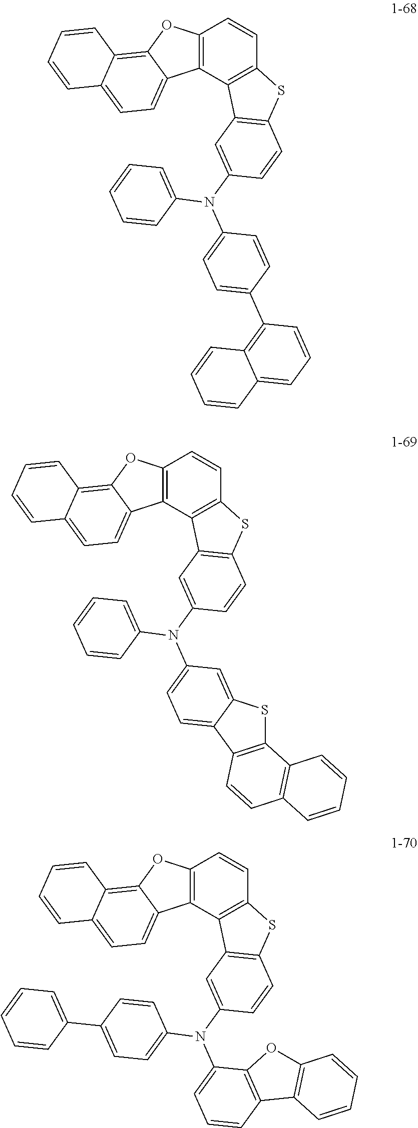 [SMILES: c1ccc(N(c2ccc(-c3cccc4ccccc34)cc2)c2ccc3sc4ccc5oc6c7ccccc7ccc6c5c4c3c2)cc1 ; c1ccc(N(c2ccc3c(c2)sc2c4ccccc4ccc32)c2ccc3sc4ccc5oc6c7ccccc7ccc6c5c4c3c2)cc1 ; c1ccc(-c2ccc(N(c3ccc4sc5ccc6oc7c8ccccc8ccc7c6c5c4c3)c3cccc4c3oc3ccccc34)cc2)cc1]

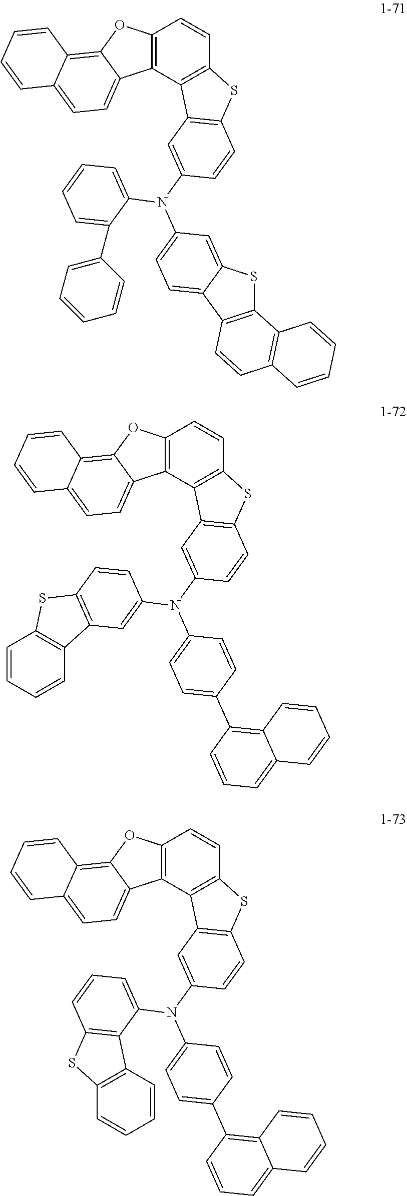 [SMILES: c1ccc(-c2ccccc2N(c2ccc3c(c2)sc2c4ccccc4ccc32)c2ccc3sc4ccc5oc6c7ccccc7ccc6c5c4c3c2)cc1 ; c1ccc2c(-c3ccc(N(c4ccc5sc6ccccc6c5c4)c4ccc5sc6ccc7oc8c9ccccc9ccc8c7c6c5c4)cc3)cccc2c1 ; c1ccc2c(-c3ccc(N(c4ccc5sc6ccc7oc8c9ccccc9ccc8c7c6c5c4)c4cccc5sc6ccccc6c45)cc3)cccc2c1]

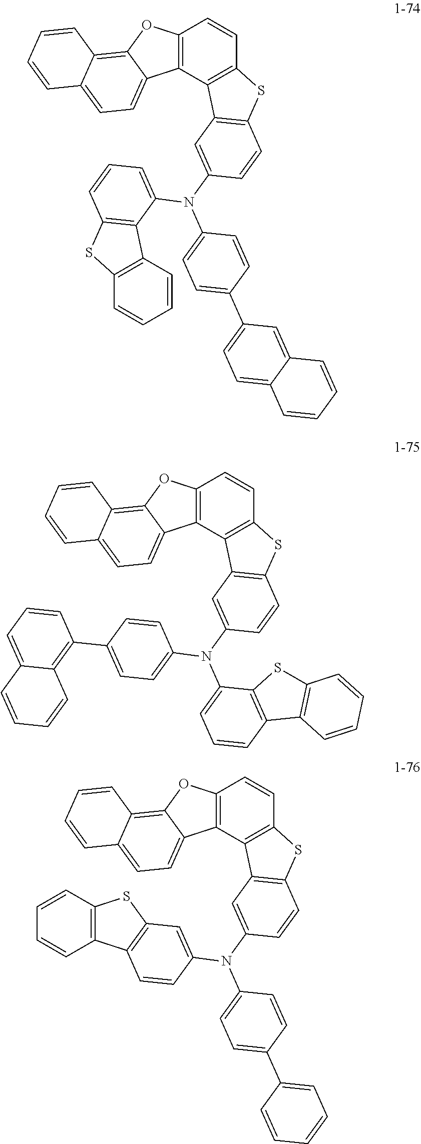

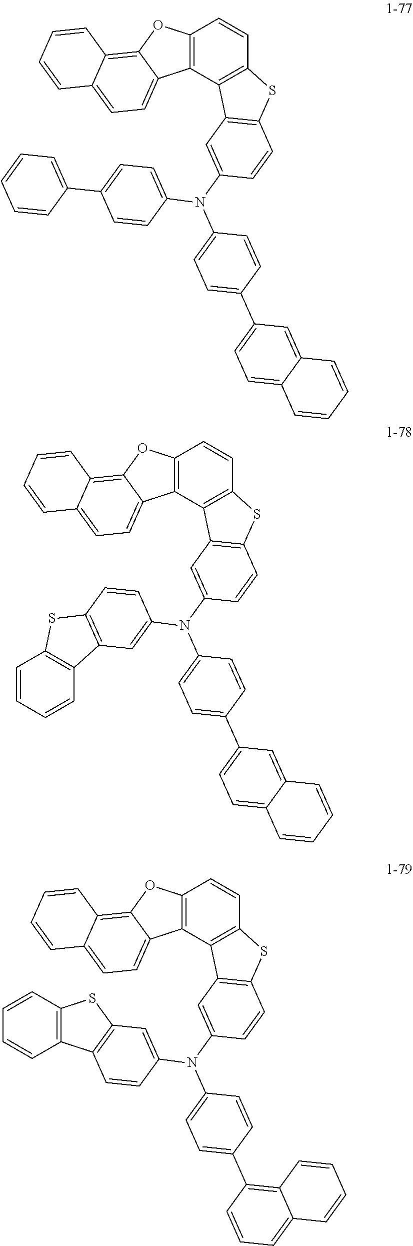 [SMILES: c1ccc(-c2ccc(N(c3ccc(-c4ccc5ccccc5c4)cc3)c3ccc4sc5ccc6oc7c8ccccc8ccc7c6c5c4c3)cc2)cc1 ; c1ccc2cc(-c3ccc(N(c4ccc5sc6ccccc6c5c4)c4ccc5sc6ccc7oc8c9ccccc9ccc8c7c6c5c4)cc3)ccc2c1 ; c1ccc2c(-c3ccc(N(c4ccc5c(c4)sc4ccccc45)c4ccc5sc6ccc7oc8c9ccccc9ccc8c7c6c5c4)cc3)cccc2c1]

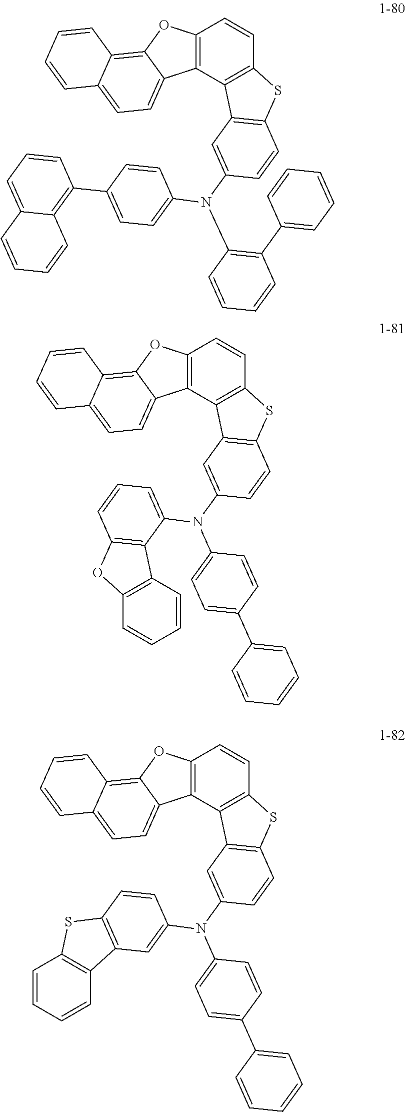

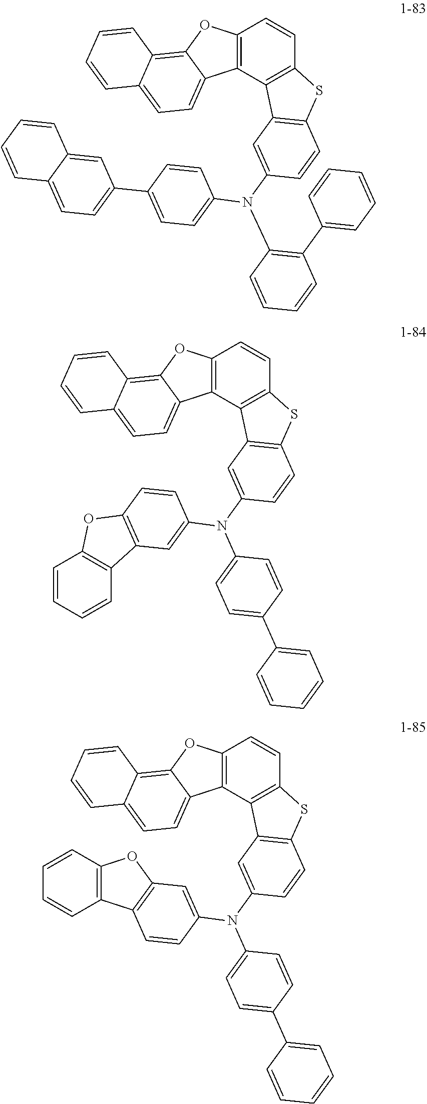

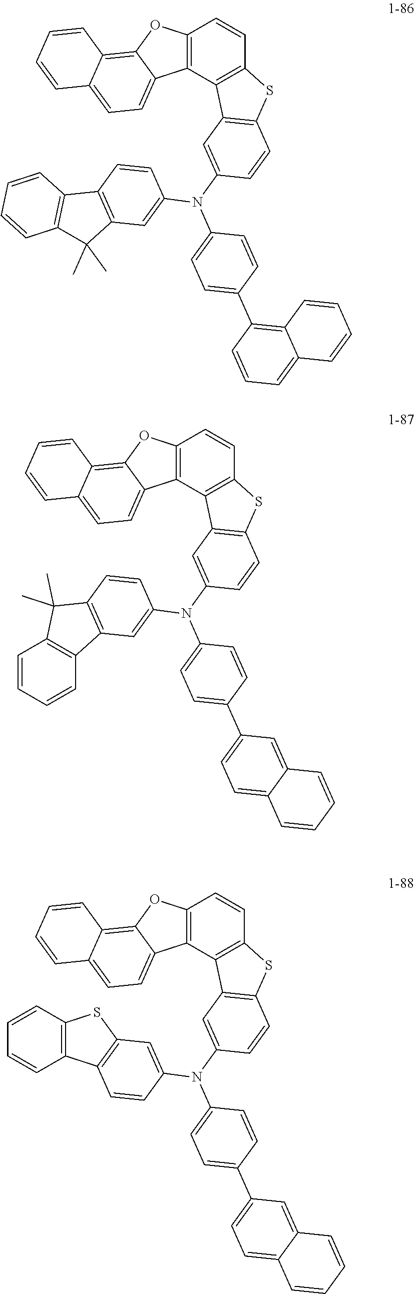 [SMILES: CC1(C)c2ccccc2-c2ccc(N(c3ccc(-c4cccc5ccccc45)cc3)c3ccc4sc5ccc6oc7c8ccccc8ccc7c6c5c4c3)cc21 ; CC1(C)c2ccccc2-c2cc(N(c3ccc(-c4ccc5ccccc5c4)cc3)c3ccc4sc5ccc6oc7c8ccccc8ccc7c6c5c4c3)ccc21 ; c1ccc2cc(-c3ccc(N(c4ccc5c(c4)sc4ccccc45)c4ccc5sc6ccc7oc8c9ccccc9ccc8c7c6c5c4)cc3)ccc2c1]

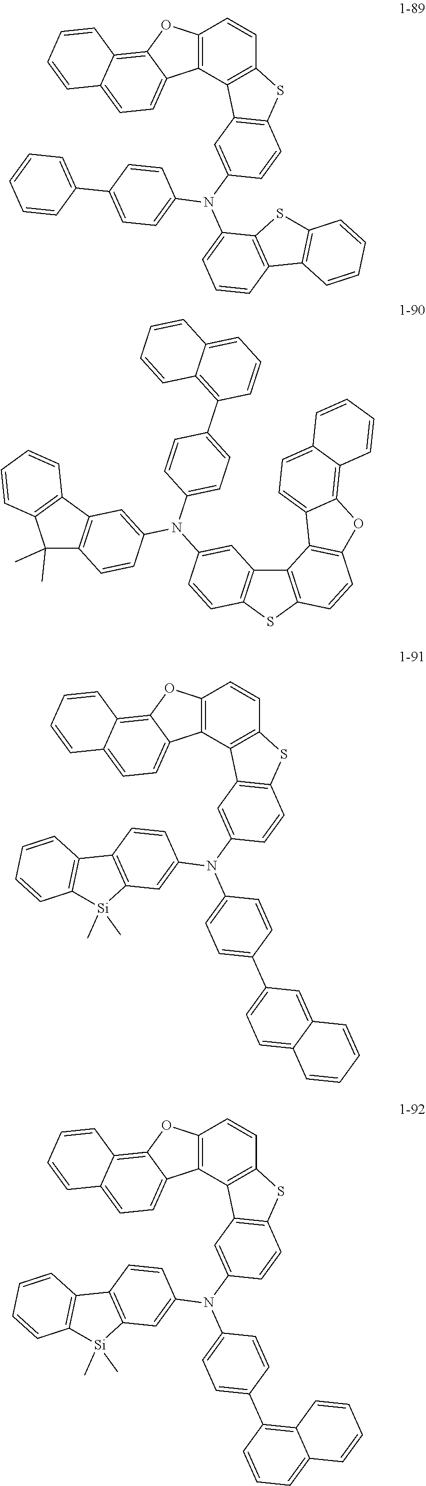

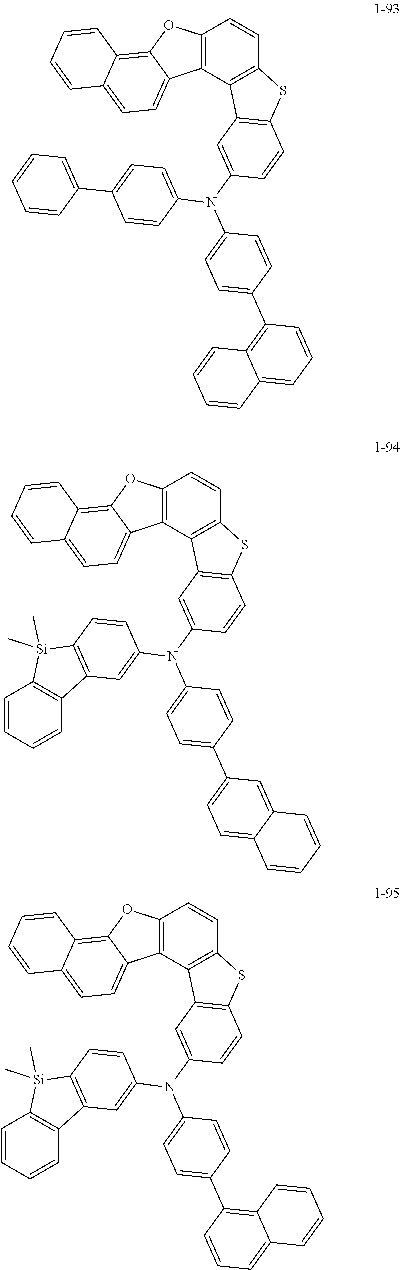 [SMILES: c1ccc(-c2ccc(N(c3ccc(-c4cccc5ccccc45)cc3)c3ccc4sc5ccc6oc7c8ccccc8ccc7c6c5c4c3)cc2)cc1 ; C[Si]1(C)c2ccccc2-c2cc(N(c3ccc(-c4ccc5ccccc5c4)cc3)c3ccc4sc5ccc6oc7c8ccccc8ccc7c6c5c4c3)ccc21 ; C[Si]1(C)c2ccccc2-c2cc(N(c3ccc(-c4cccc5ccccc45)cc3)c3ccc4sc5ccc6oc7c8ccccc8ccc7c6c5c4c3)ccc21]

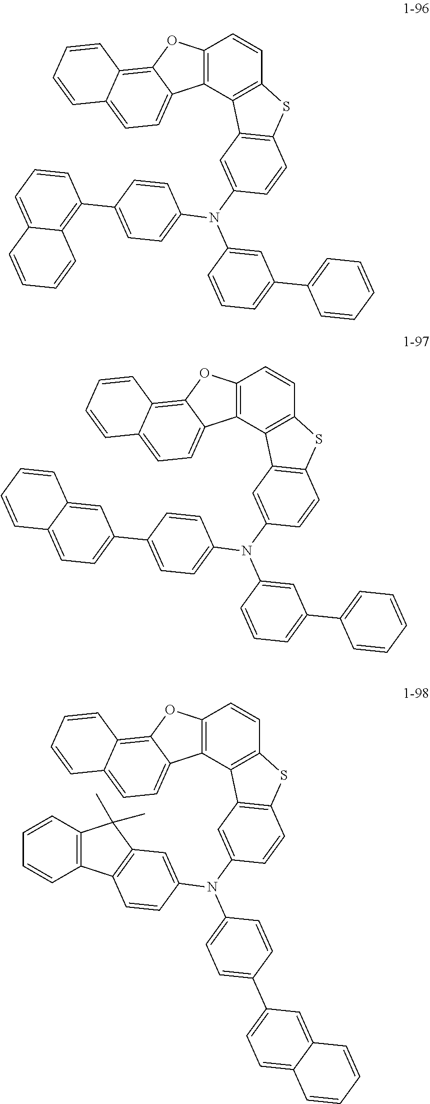 [SMILES: c1ccc(-c2cccc(N(c3ccc(-c4cccc5ccccc45)cc3)c3ccc4sc5ccc6oc7c8ccccc8ccc7c6c5c4c3)c2)cc1 ; c1ccc(-c2cccc(N(c3ccc(-c4ccc5ccccc5c4)cc3)c3ccc4sc5ccc6oc7c8ccccc8ccc7c6c5c4c3)c2)cc1 ; CC1(C)c2ccccc2-c2ccc(N(c3ccc(-c4ccc5ccccc5c4)cc3)c3ccc4sc5ccc6oc7c8ccccc8ccc7c6c5c4c3)cc21]

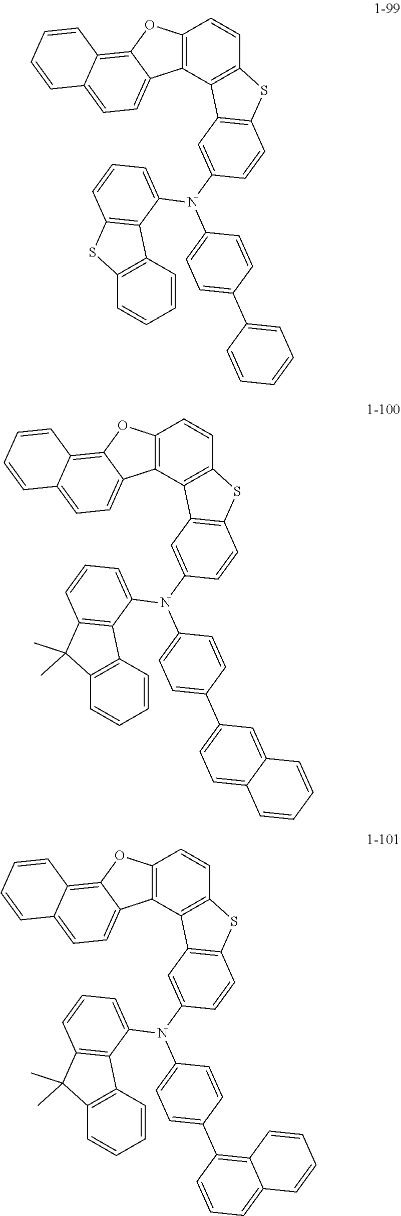 [SMILES: c1ccc(-c2ccc(N(c3ccc4sc5ccc6oc7c8ccccc8ccc7c6c5c4c3)c3cccc4sc5ccccc5c34)cc2)cc1 ; CC1(C)c2ccccc2-c2c(N(c3ccc(-c4ccc5ccccc5c4)cc3)c3ccc4sc5ccc6oc7c8ccccc8ccc7c6c5c4c3)cccc21 ; CC1(C)c2ccccc2-c2c(N(c3ccc(-c4cccc5ccccc45)cc3)c3ccc4sc5ccc6oc7c8ccccc8ccc7c6c5c4c3)cccc21]

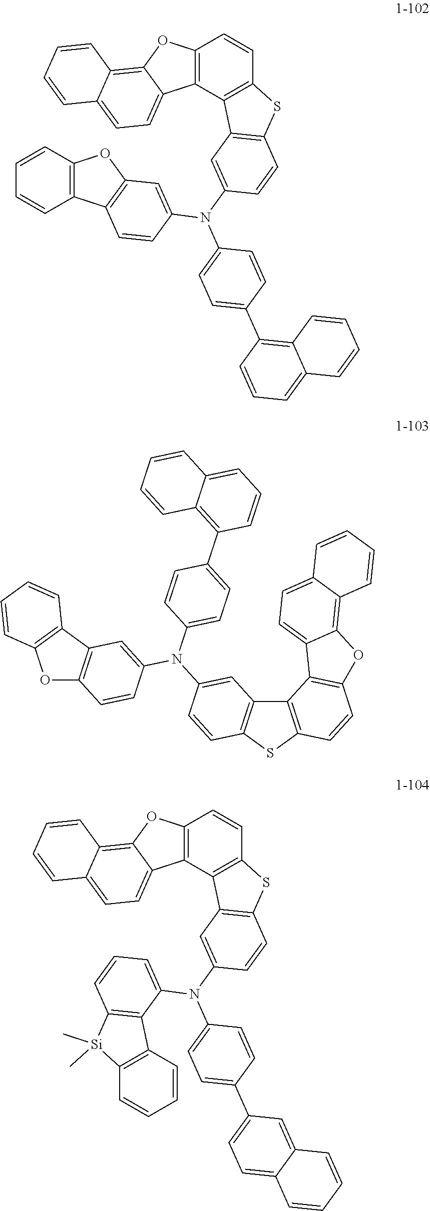 [SMILES: c1ccc2c(-c3ccc(N(c4ccc5c(c4)oc4ccccc45)c4ccc5sc6ccc7oc8c9ccccc9ccc8c7c6c5c4)cc3)cccc2c1 ; c1ccc2c(-c3ccc(N(c4ccc5oc6ccccc6c5c4)c4ccc5sc6ccc7oc8c9ccccc9ccc8c7c6c5c4)cc3)cccc2c1 ; C[Si]1(C)c2ccccc2-c2c(N(c3ccc(-c4ccc5ccccc5c4)cc3)c3ccc4sc5ccc6oc7c8ccccc8ccc7c6c5c4c3)cccc21]

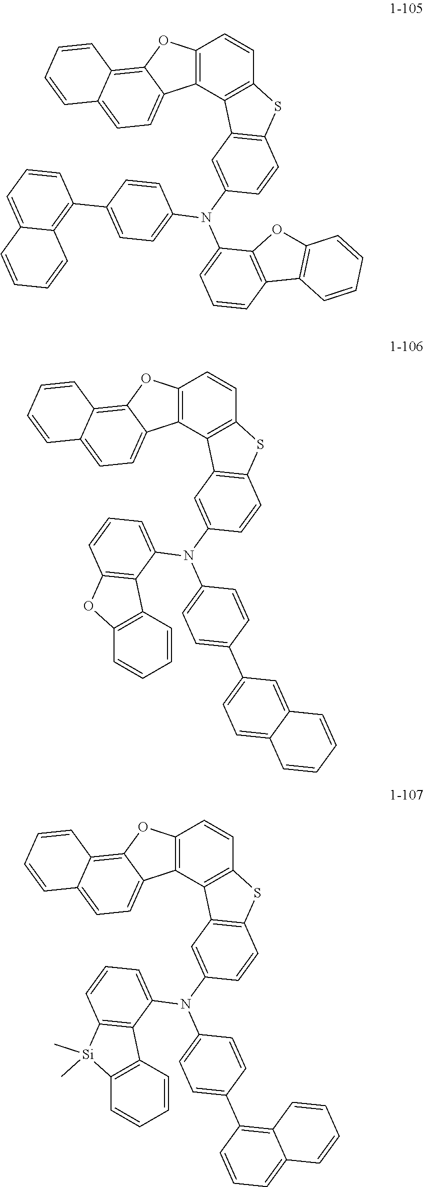 [SMILES: c1ccc2c(-c3ccc(N(c4ccc5sc6ccc7oc8c9ccccc9ccc8c7c6c5c4)c4cccc5c4oc4ccccc45)cc3)cccc2c1 ; c1ccc2cc(-c3ccc(N(c4ccc5sc6ccc7oc8c9ccccc9ccc8c7c6c5c4)c4cccc5oc6ccccc6c45)cc3)ccc2c1 ; C[Si]1(C)c2ccccc2-c2c(N(c3ccc(-c4cccc5ccccc45)cc3)c3ccc4sc5ccc6oc7c8ccccc8ccc7c6c5c4c3)cccc21]

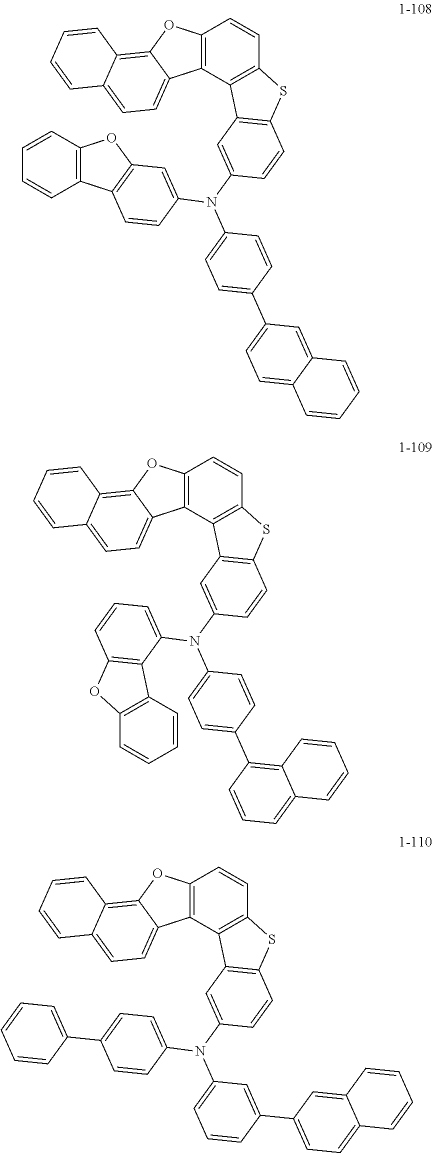 [SMILES: c1ccc2cc(-c3ccc(N(c4ccc5c(c4)oc4ccccc45)c4ccc5sc6ccc7oc8c9ccccc9ccc8c7c6c5c4)cc3)ccc2c1 ; c1ccc2c(-c3ccc(N(c4ccc5sc6ccc7oc8c9ccccc9ccc8c7c6c5c4)c4cccc5oc6ccccc6c45)cc3)cccc2c1 ; c1ccc(-c2ccc(N(c3cccc(-c4ccc5ccccc5c4)c3)c3ccc4sc5ccc6oc7c8ccccc8ccc7c6c5c4c3)cc2)cc1]

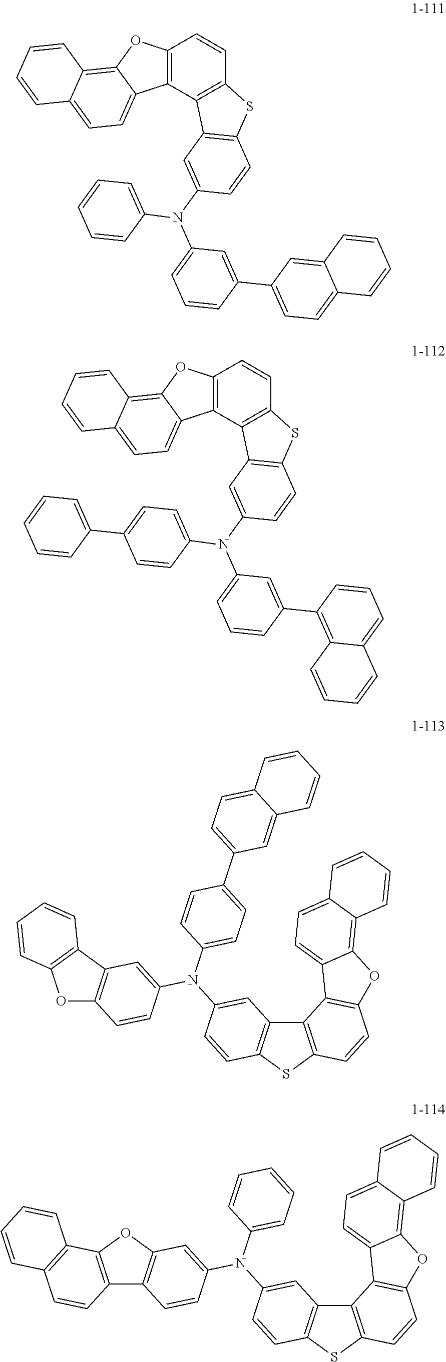 [SMILES: c1ccc(N(c2cccc(-c3ccc4ccccc4c3)c2)c2ccc3sc4ccc5oc6c7ccccc7ccc6c5c4c3c2)cc1 ; c1ccc(-c2ccc(N(c3cccc(-c4cccc5ccccc45)c3)c3ccc4sc5ccc6oc7c8ccccc8ccc7c6c5c4c3)cc2)cc1 ; c1ccc2cc(-c3ccc(N(c4ccc5oc6ccccc6c5c4)c4ccc5sc6ccc7oc8c9ccccc9ccc8c7c6c5c4)cc3)ccc2c1 ; c1ccc(N(c2ccc3c(c2)oc2c4ccccc4ccc32)c2ccc3sc4ccc5oc6c7ccccc7ccc6c5c4c3c2)cc1]

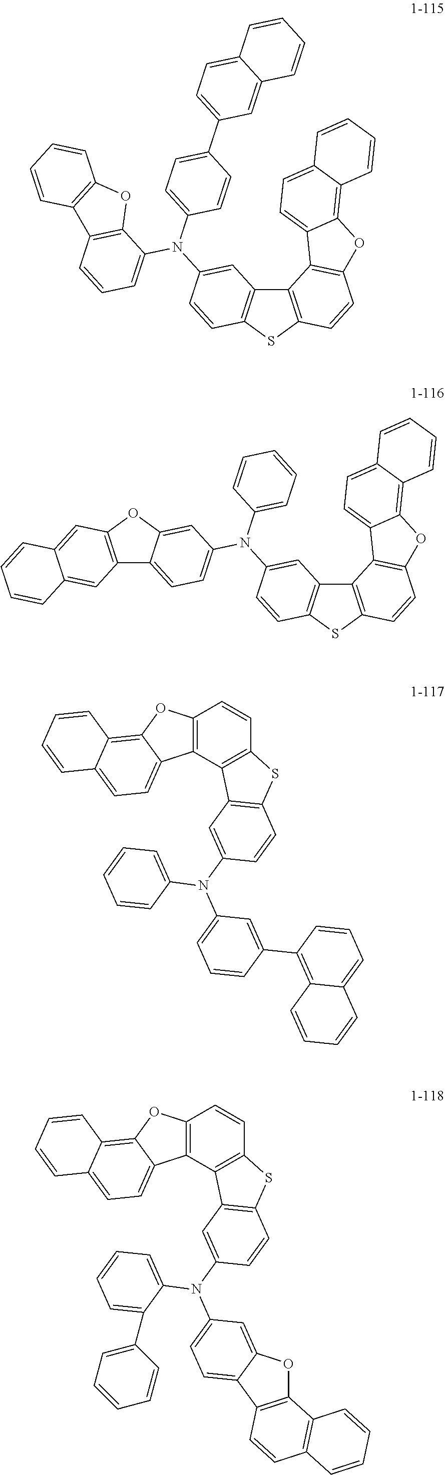 [SMILES: c1ccc2cc(-c3ccc(N(c4ccc5sc6ccc7oc8c9ccccc9ccc8c7c6c5c4)c4cccc5c4oc4ccccc45)cc3)ccc2c1 ; c1ccc(N(c2ccc3c(c2)oc2cc4ccccc4cc23)c2ccc3sc4ccc5oc6c7ccccc7ccc6c5c4c3c2)cc1 ; c1ccc(N(c2cccc(-c3cccc4ccccc34)c2)c2ccc3sc4ccc5oc6c7ccccc7ccc6c5c4c3c2)cc1 ; c1ccc(-c2ccccc2N(c2ccc3c(c2)oc2c4ccccc4ccc32)c2ccc3sc4ccc5oc6c7ccccc7ccc6c5c4c3c2)cc1]

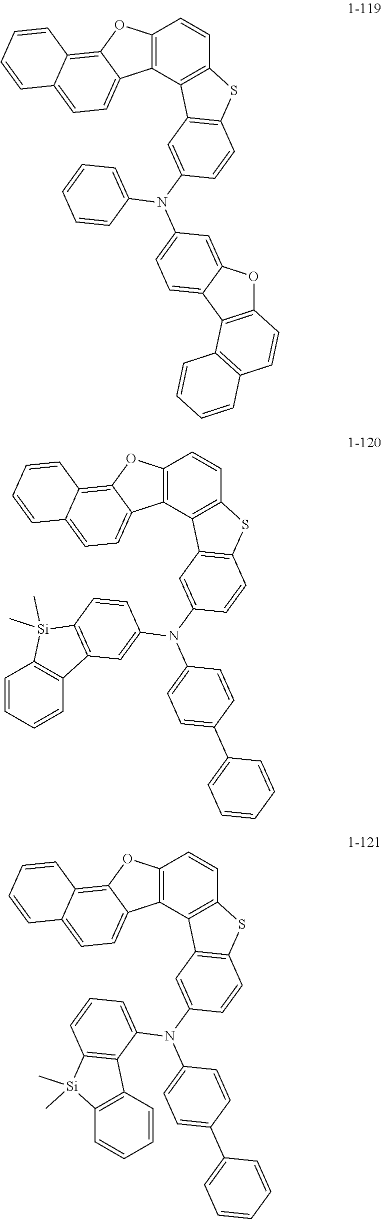

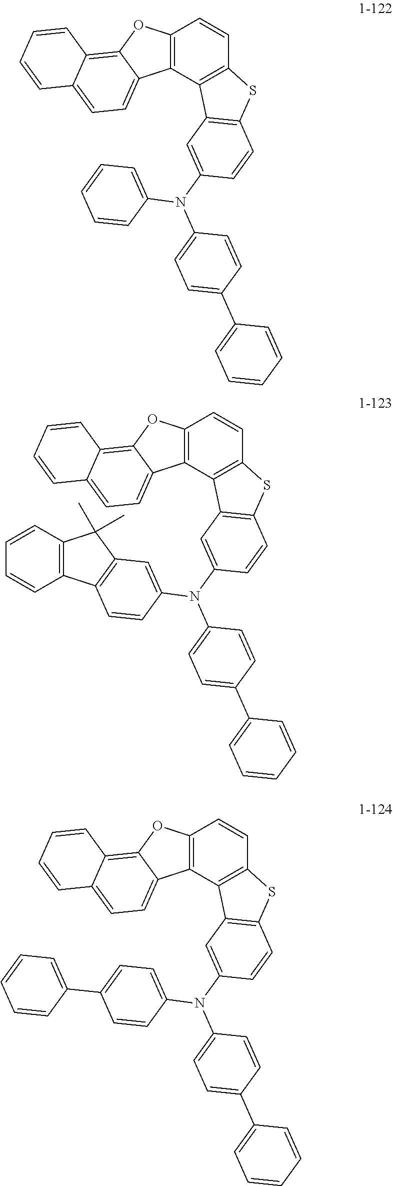

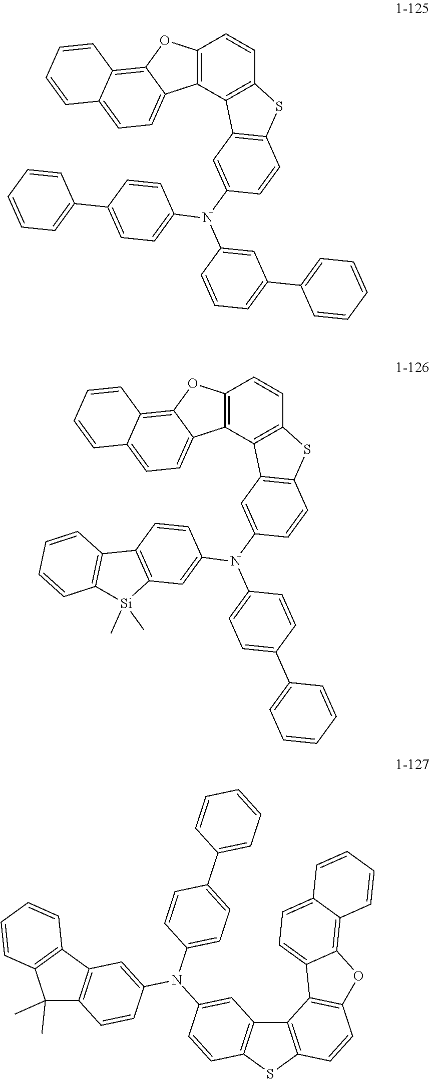 [SMILES: c1ccc(-c2ccc(N(c3cccc(-c4ccccc4)c3)c3ccc4sc5ccc6oc7c8ccccc8ccc7c6c5c4c3)cc2)cc1 ; C[Si]1(C)c2ccccc2-c2ccc(N(c3ccc(-c4ccccc4)cc3)c3ccc4sc5ccc6oc7c8ccccc8ccc7c6c5c4c3)cc21 ; CC1(C)c2ccccc2-c2cc(N(c3ccc(-c4ccccc4)cc3)c3ccc4sc5ccc6oc7c8ccccc8ccc7c6c5c4c3)ccc21]

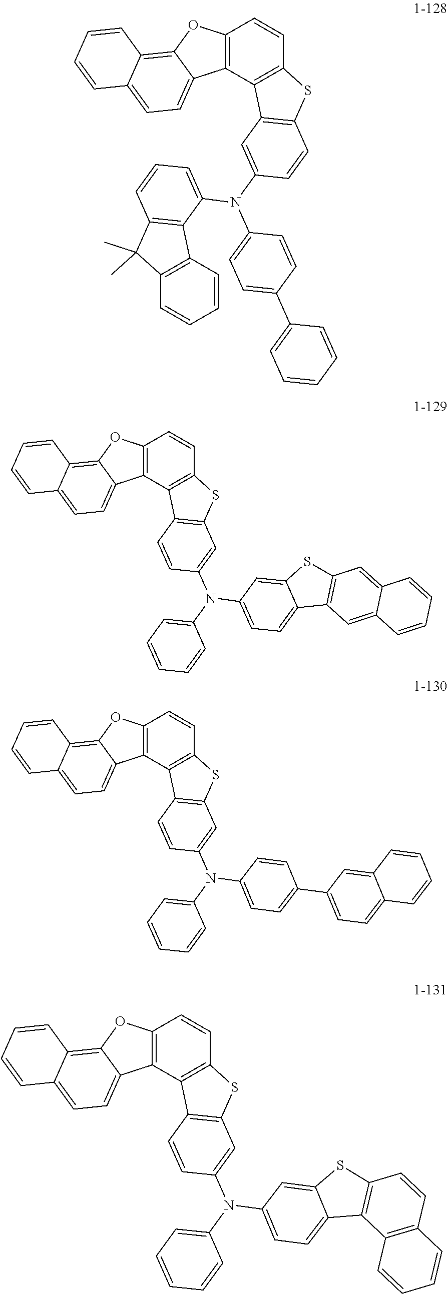

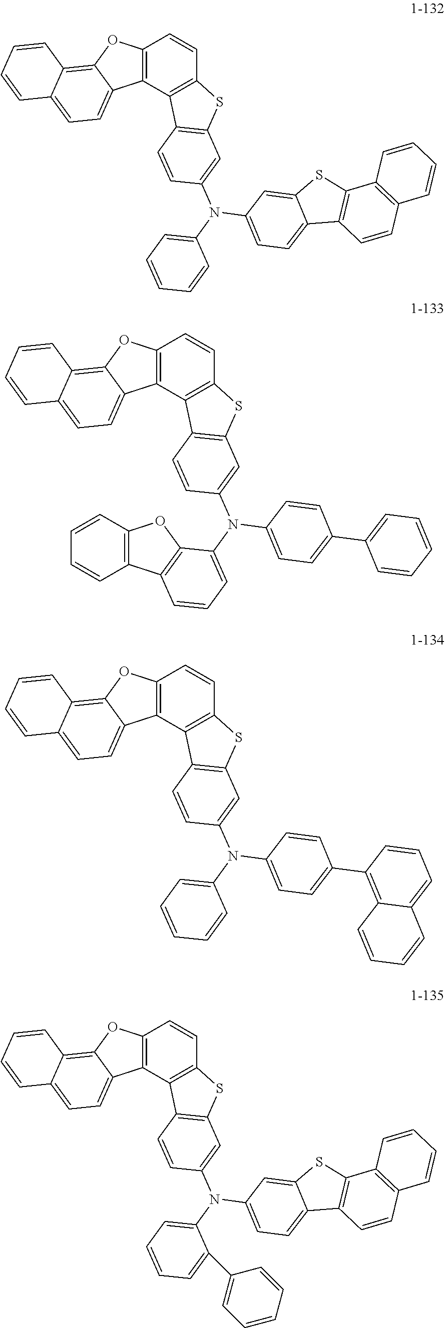

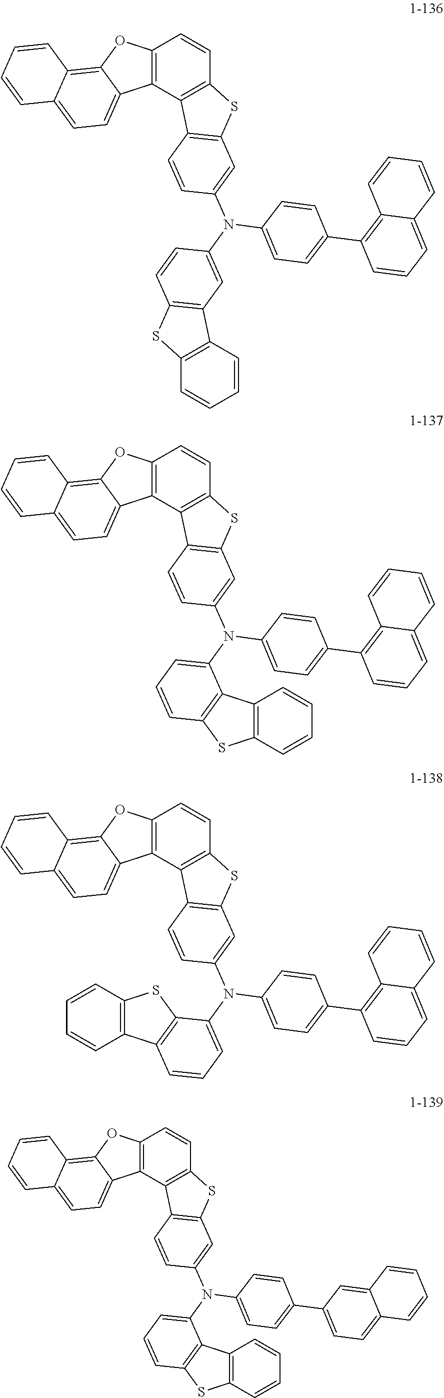 [SMILES: c1ccc2c(-c3ccc(N(c4ccc5c(c4)sc4ccc6oc7c8ccccc8ccc7c6c45)c4ccc5sc6ccccc6c5c4)cc3)cccc2c1 ; c1ccc2c(-c3ccc(N(c4ccc5c(c4)sc4ccc6oc7c8ccccc8ccc7c6c45)c4cccc5sc6ccccc6c45)cc3)cccc2c1 ; c1ccc2c(-c3ccc(N(c4ccc5c(c4)sc4ccc6oc7c8ccccc8ccc7c6c45)c4cccc5c4sc4ccccc45)cc3)cccc2c1 ; c1ccc2cc(-c3ccc(N(c4ccc5c(c4)sc4ccc6oc7c8ccccc8ccc7c6c45)c4cccc5sc6ccccc6c45)cc3)ccc2c1]

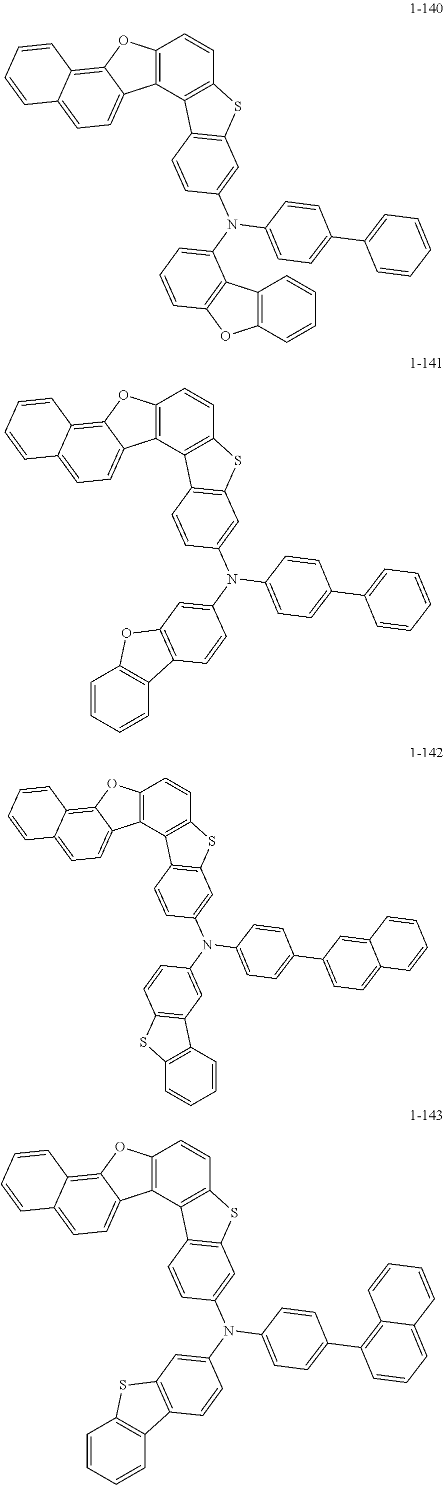

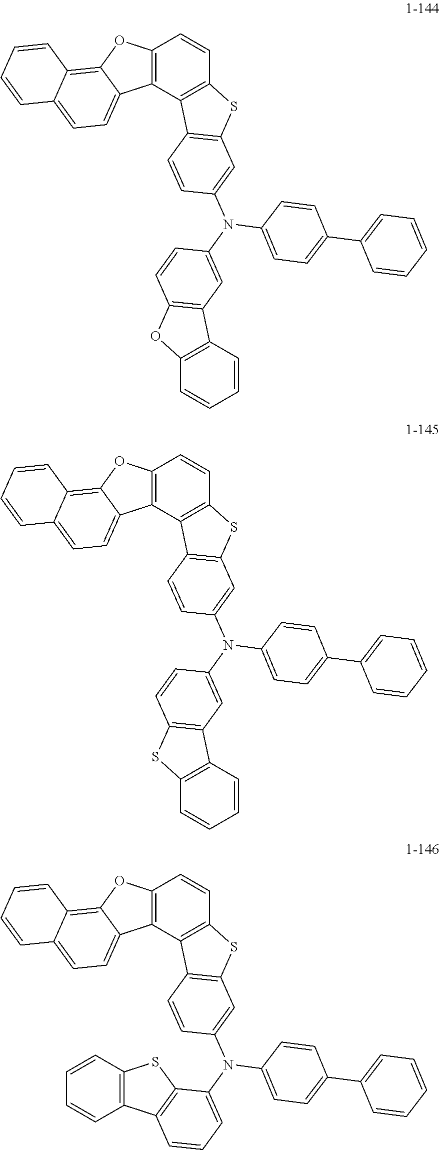 [SMILES: c1ccc(-c2ccc(N(c3ccc4c(c3)sc3ccc5oc6c7ccccc7ccc6c5c34)c3ccc4oc5ccccc5c4c3)cc2)cc1 ; c1ccc(-c2ccc(N(c3ccc4c(c3)sc3ccc5oc6c7ccccc7ccc6c5c34)c3ccc4sc5ccccc5c4c3)cc2)cc1 ; c1ccc(-c2ccc(N(c3ccc4c(c3)sc3ccc5oc6c7ccccc7ccc6c5c34)c3cccc4c3sc3ccccc34)cc2)cc1]

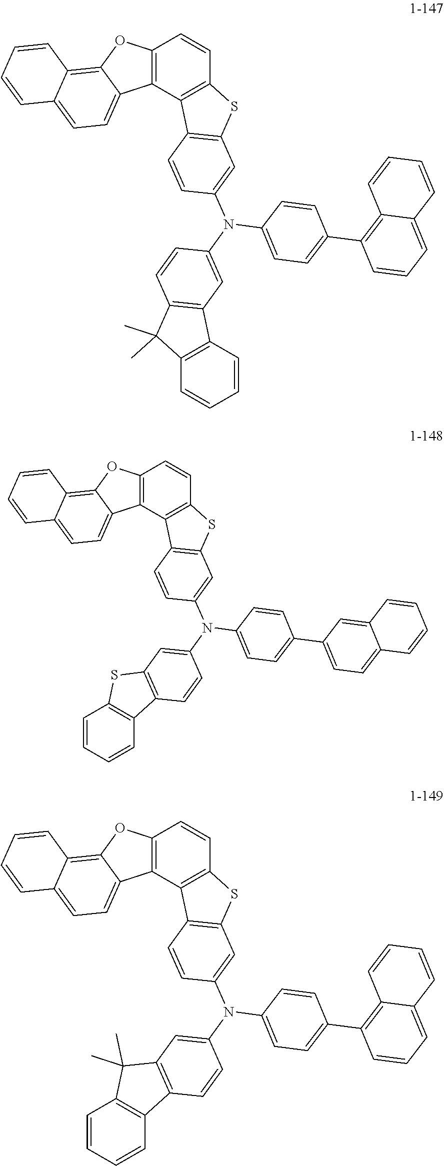 [SMILES: CC1(C)c2ccccc2-c2cc(N(c3ccc(-c4cccc5ccccc45)cc3)c3ccc4c(c3)sc3ccc5oc6c7ccccc7ccc6c5c34)ccc21 ; c1ccc2cc(-c3ccc(N(c4ccc5c(c4)sc4ccccc45)c4ccc5c(c4)sc4ccc6oc7c8ccccc8ccc7c6c45)cc3)ccc2c1 ; CC1(C)c2ccccc2-c2ccc(N(c3ccc(-c4cccc5ccccc45)cc3)c3ccc4c(c3)sc3ccc5oc6c7ccccc7ccc6c5c34)cc21]

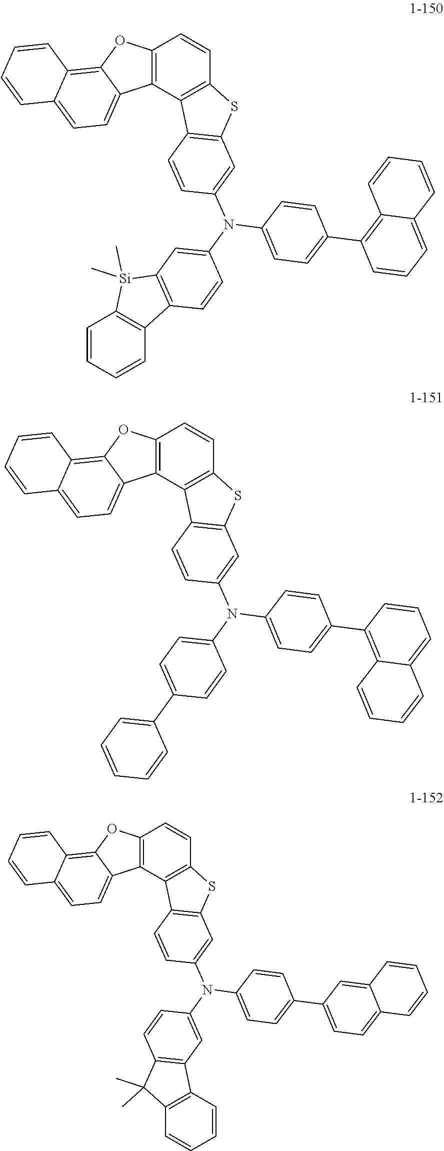 [SMILES: C[Si]1(C)c2ccccc2-c2ccc(N(c3ccc(-c4cccc5ccccc45)cc3)c3ccc4c(c3)sc3ccc5oc6c7ccccc7ccc6c5c34)cc21 ; c1ccc(-c2ccc(N(c3ccc(-c4cccc5ccccc45)cc3)c3ccc4c(c3)sc3ccc5oc6c7ccccc7ccc6c5c34)cc2)cc1 ; CC1(C)c2ccccc2-c2cc(N(c3ccc(-c4ccc5ccccc5c4)cc3)c3ccc4c(c3)sc3ccc5oc6c7ccccc7ccc6c5c34)ccc21]

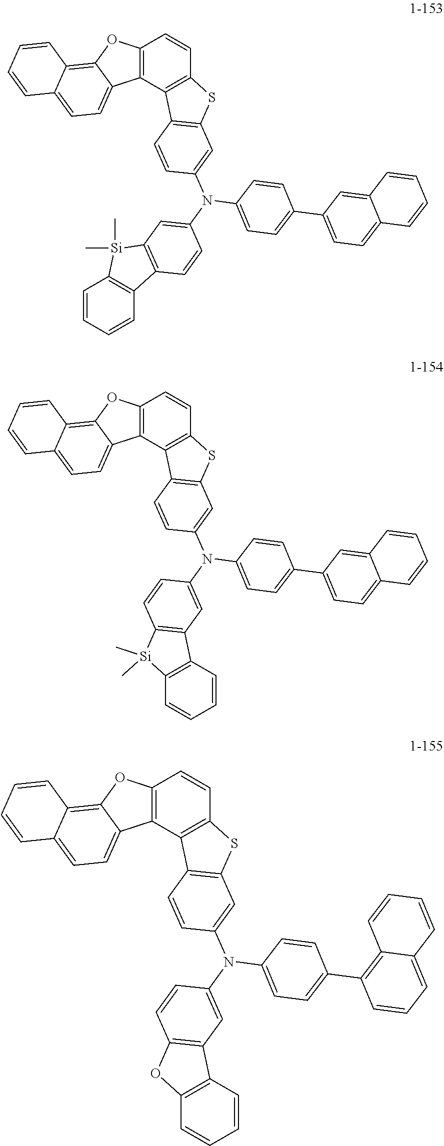 [SMILES: C[Si]1(C)c2ccccc2-c2ccc(N(c3ccc(-c4ccc5ccccc5c4)cc3)c3ccc4c(c3)sc3ccc5oc6c7ccccc7ccc6c5c34)cc21 ; C[Si]1(C)c2ccccc2-c2cc(N(c3ccc(-c4ccc5ccccc5c4)cc3)c3ccc4c(c3)sc3ccc5oc6c7ccccc7ccc6c5c34)ccc21 ; c1ccc2c(-c3ccc(N(c4ccc5c(c4)sc4ccc6oc7c8ccccc8ccc7c6c45)c4ccc5oc6ccccc6c5c4)cc3)cccc2c1]

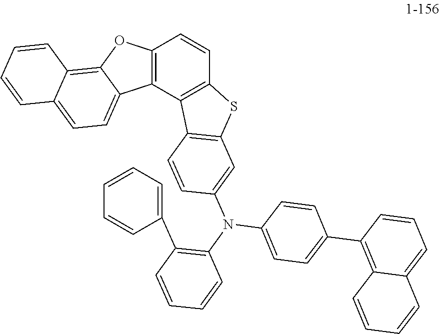 [SMILES: c1ccc(-c2ccccc2N(c2ccc(-c3cccc4ccccc34)cc2)c2ccc3c(c2)sc2ccc4oc5c6ccccc6ccc5c4c23)cc1]

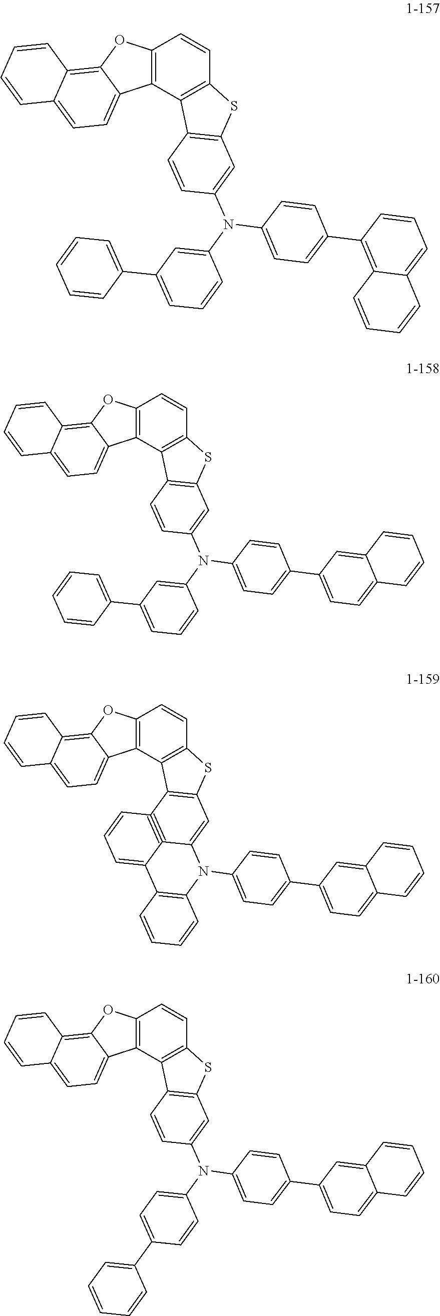

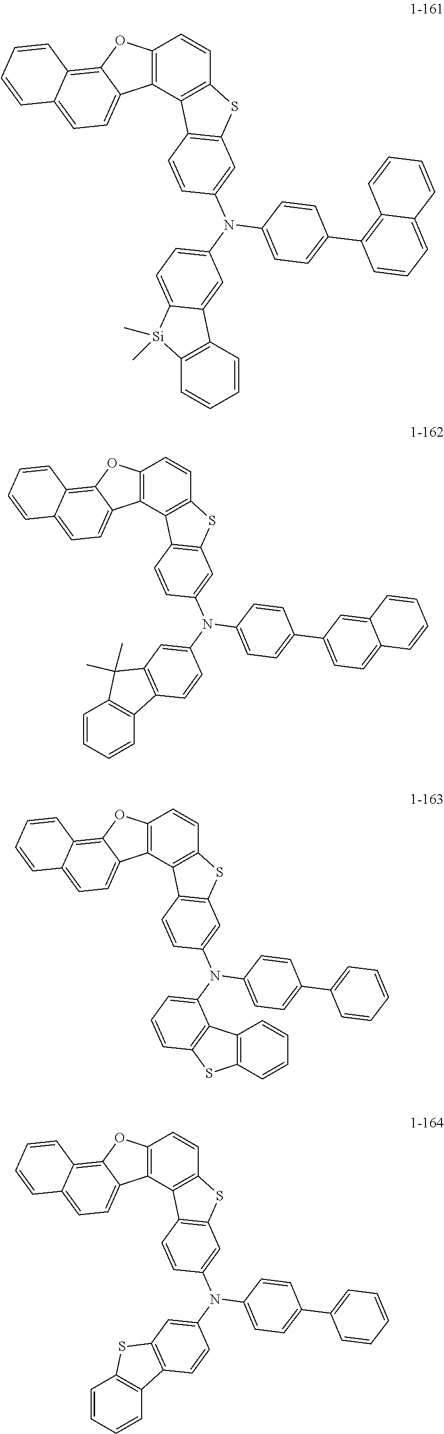 [SMILES: C[Si]1(C)c2ccccc2-c2cc(N(c3ccc(-c4cccc5ccccc45)cc3)c3ccc4c(c3)sc3ccc5oc6c7ccccc7ccc6c5c34)ccc21 ; CC1(C)c2ccccc2-c2ccc(N(c3ccc(-c4ccc5ccccc5c4)cc3)c3ccc4c(c3)sc3ccc5oc6c7ccccc7ccc6c5c34)cc21 ; c1ccc(-c2ccc(N(c3ccc4c(c3)sc3ccc5oc6c7ccccc7ccc6c5c34)c3cccc4sc5ccccc5c34)cc2)cc1 ; c1ccc(-c2ccc(N(c3ccc4c(c3)sc3ccccc34)c3ccc4c(c3)sc3ccc5oc6c7ccccc7ccc6c5c34)cc2)cc1]

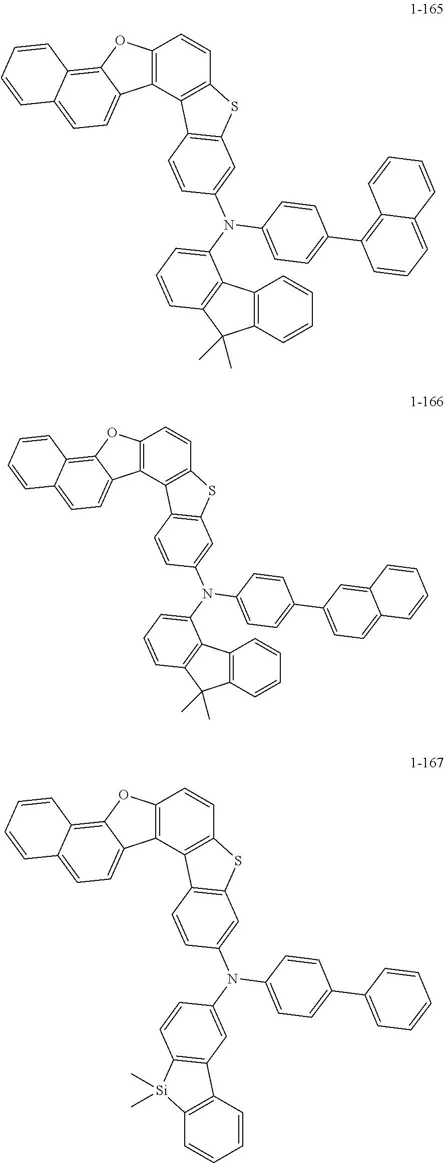 [SMILES: CC1(C)c2ccccc2-c2c(N(c3ccc(-c4cccc5ccccc45)cc3)c3ccc4c(c3)sc3ccc5oc6c7ccccc7ccc6c5c34)cccc21 ; CC1(C)c2ccccc2-c2c(N(c3ccc(-c4ccc5ccccc5c4)cc3)c3ccc4c(c3)sc3ccc5oc6c7ccccc7ccc6c5c34)cccc21 ; C[Si]1(C)c2ccccc2-c2cc(N(c3ccc(-c4ccccc4)cc3)c3ccc4c(c3)sc3ccc5oc6c7ccccc7ccc6c5c34)ccc21]

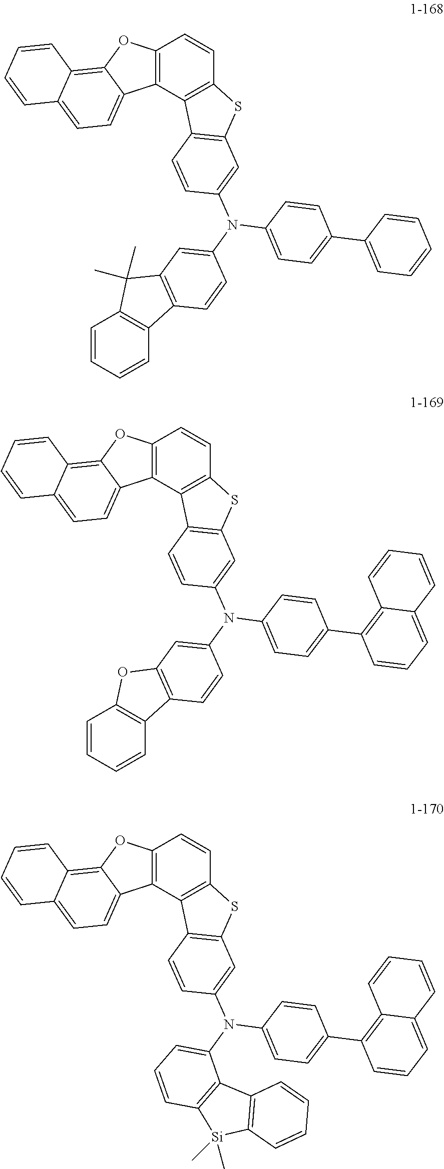 [SMILES: CC1(C)c2ccccc2-c2ccc(N(c3ccc(-c4ccccc4)cc3)c3ccc4c(c3)sc3ccc5oc6c7ccccc7ccc6c5c34)cc21 ; c1ccc2c(-c3ccc(N(c4ccc5c(c4)oc4ccccc45)c4ccc5c(c4)sc4ccc6oc7c8ccccc8ccc7c6c45)cc3)cccc2c1 ; C[Si]1(C)c2ccccc2-c2c(N(c3ccc(-c4cccc5ccccc45)cc3)c3ccc4c(c3)sc3ccc5oc6c7ccccc7ccc6c5c34)cccc21]

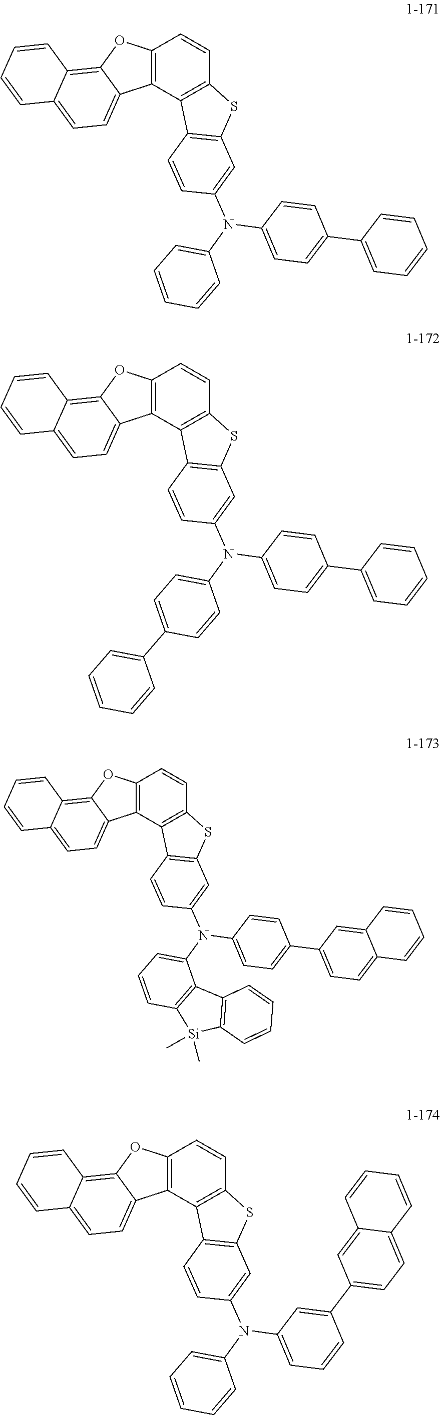 [SMILES: c1ccc(-c2ccc(N(c3ccccc3)c3ccc4c(c3)sc3ccc5oc6c7ccccc7ccc6c5c34)cc2)cc1 ; c1ccc(-c2ccc(N(c3ccc(-c4ccccc4)cc3)c3ccc4c(c3)sc3ccc5oc6c7ccccc7ccc6c5c34)cc2)cc1 ; C[Si]1(C)c2ccccc2-c2c(N(c3ccc(-c4ccc5ccccc5c4)cc3)c3ccc4c(c3)sc3ccc5oc6c7ccccc7ccc6c5c34)cccc21 ; c1ccc(N(c2cccc(-c3ccc4ccccc4c3)c2)c2ccc3c(c2)sc2ccc4oc5c6ccccc6ccc5c4c23)cc1]

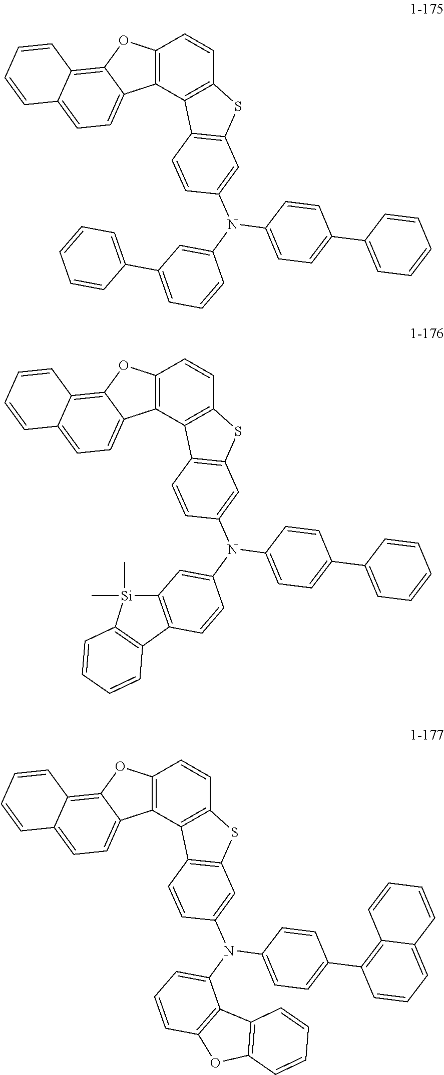 [SMILES: c1ccc(-c2ccc(N(c3cccc(-c4ccccc4)c3)c3ccc4c(c3)sc3ccc5oc6c7ccccc7ccc6c5c34)cc2)cc1 ; C[Si]1(C)c2ccccc2-c2ccc(N(c3ccc(-c4ccccc4)cc3)c3ccc4c(c3)sc3ccc5oc6c7ccccc7ccc6c5c34)cc21 ; c1ccc2c(-c3ccc(N(c4ccc5c(c4)sc4ccc6oc7c8ccccc8ccc7c6c45)c4cccc5oc6ccccc6c45)cc3)cccc2c1]

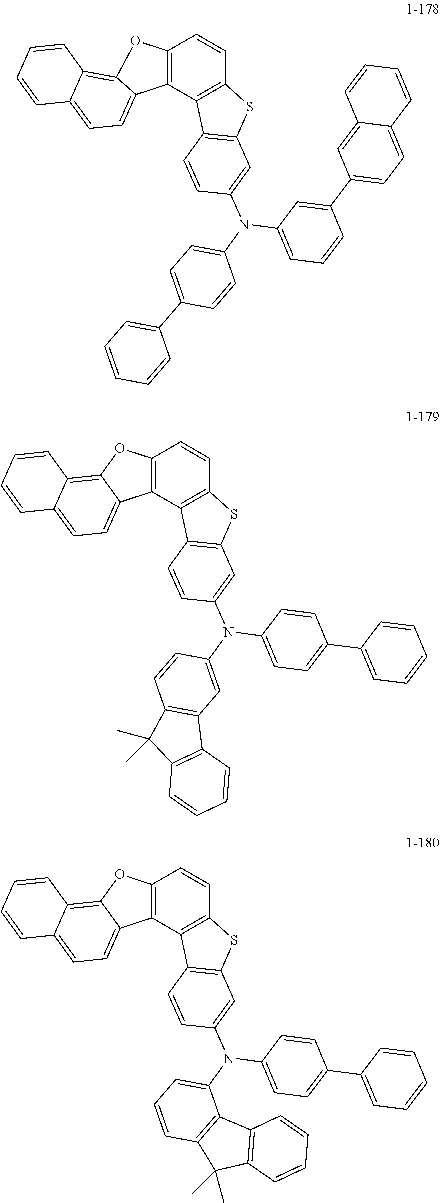 [SMILES: c1ccc(-c2ccc(N(c3cccc(-c4ccc5ccccc5c4)c3)c3ccc4c(c3)sc3ccc5oc6c7ccccc7ccc6c5c34)cc2)cc1 ; CC1(C)c2ccccc2-c2cc(N(c3ccc(-c4ccccc4)cc3)c3ccc4c(c3)sc3ccc5oc6c7ccccc7ccc6c5c34)ccc21 ; CC1(C)c2ccccc2-c2c(N(c3ccc(-c4ccccc4)cc3)c3ccc4c(c3)sc3ccc5oc6c7ccccc7ccc6c5c34)cccc21]

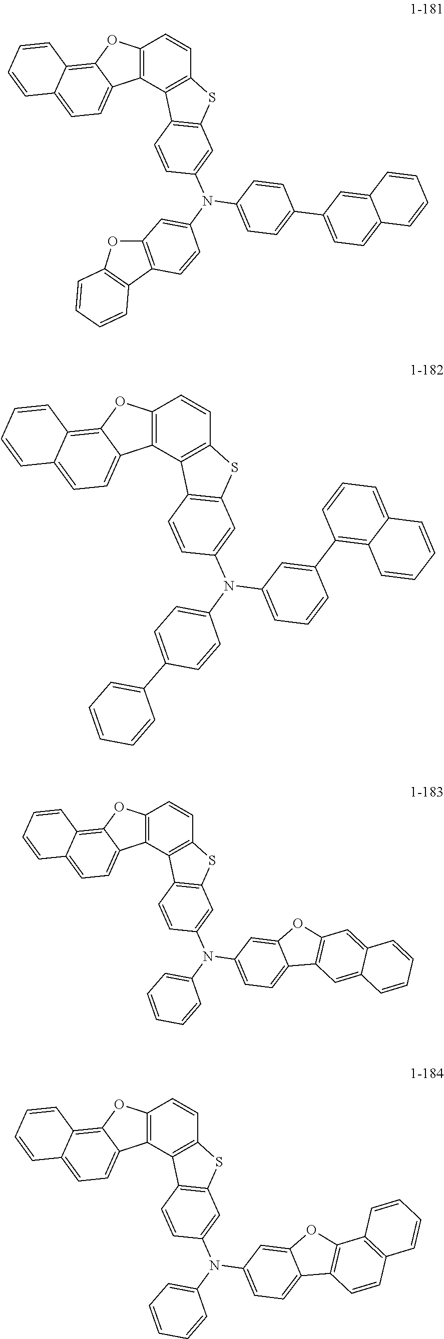 [SMILES: c1ccc2cc(-c3ccc(N(c4ccc5c(c4)oc4ccccc45)c4ccc5c(c4)sc4ccc6oc7c8ccccc8ccc7c6c45)cc3)ccc2c1 ; c1ccc(-c2ccc(N(c3cccc(-c4cccc5ccccc45)c3)c3ccc4c(c3)sc3ccc5oc6c7ccccc7ccc6c5c34)cc2)cc1 ; c1ccc(N(c2ccc3c(c2)oc2cc4ccccc4cc23)c2ccc3c(c2)sc2ccc4oc5c6ccccc6ccc5c4c23)cc1 ; c1ccc(N(c2ccc3c(c2)oc2c4ccccc4ccc32)c2ccc3c(c2)sc2ccc4oc5c6ccccc6ccc5c4c23)cc1]

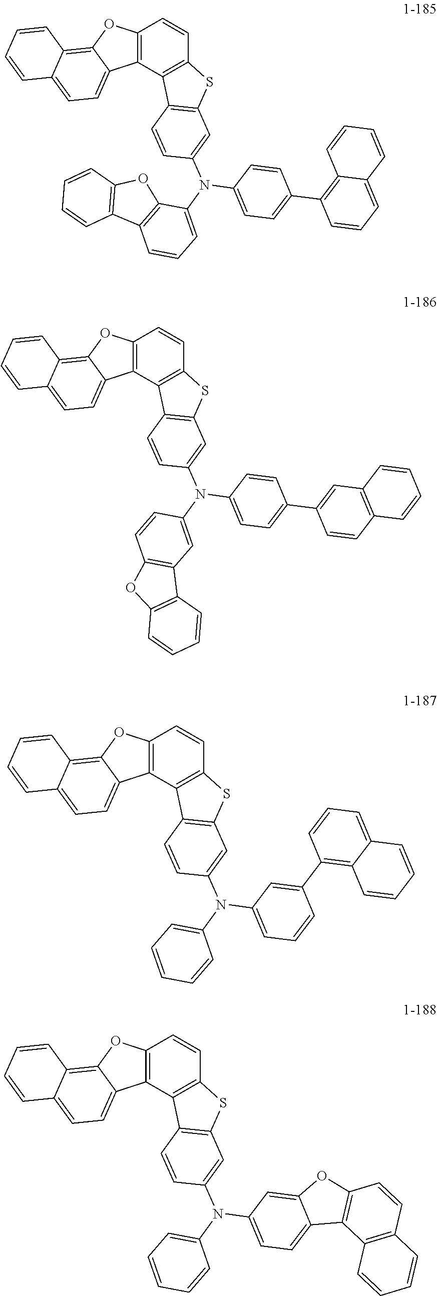 [SMILES: c1ccc2c(-c3ccc(N(c4ccc5c(c4)sc4ccc6oc7c8ccccc8ccc7c6c45)c4cccc5c4oc4ccccc45)cc3)cccc2c1 ; c1ccc2cc(-c3ccc(N(c4ccc5c(c4)sc4ccc6oc7c8ccccc8ccc7c6c45)c4ccc5oc6ccccc6c5c4)cc3)ccc2c1 ; c1ccc(N(c2cccc(-c3cccc4ccccc34)c2)c2ccc3c(c2)sc2ccc4oc5c6ccccc6ccc5c4c23)cc1 ; c1ccc(N(c2ccc3c(c2)oc2ccc4ccccc4c23)c2ccc3c(c2)sc2ccc4oc5c6ccccc6ccc5c4c23)cc1]

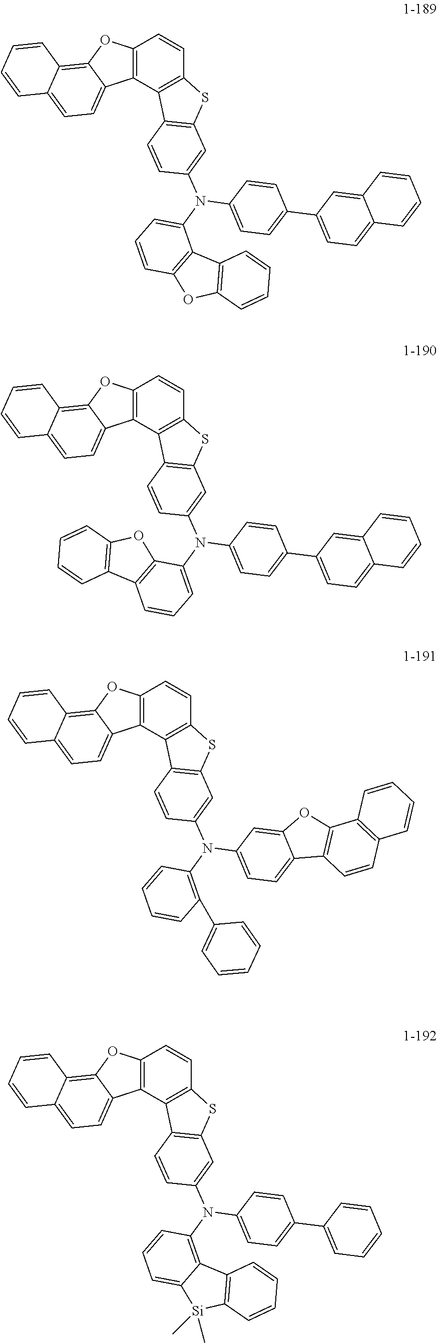 [SMILES: c1ccc2cc(-c3ccc(N(c4ccc5c(c4)sc4ccc6oc7c8ccccc8ccc7c6c45)c4cccc5oc6ccccc6c45)cc3)ccc2c1 ; c1ccc2cc(-c3ccc(N(c4ccc5c(c4)sc4ccc6oc7c8ccccc8ccc7c6c45)c4cccc5c4oc4ccccc45)cc3)ccc2c1 ; c1ccc(-c2ccccc2N(c2ccc3c(c2)oc2c4ccccc4ccc32)c2ccc3c(c2)sc2ccc4oc5c6ccccc6ccc5c4c23)cc1 ; C[Si]1(C)c2ccccc2-c2c(N(c3ccc(-c4ccccc4)cc3)c3ccc4c(c3)sc3ccc5oc6c7ccccc7ccc6c5c34)cccc21]

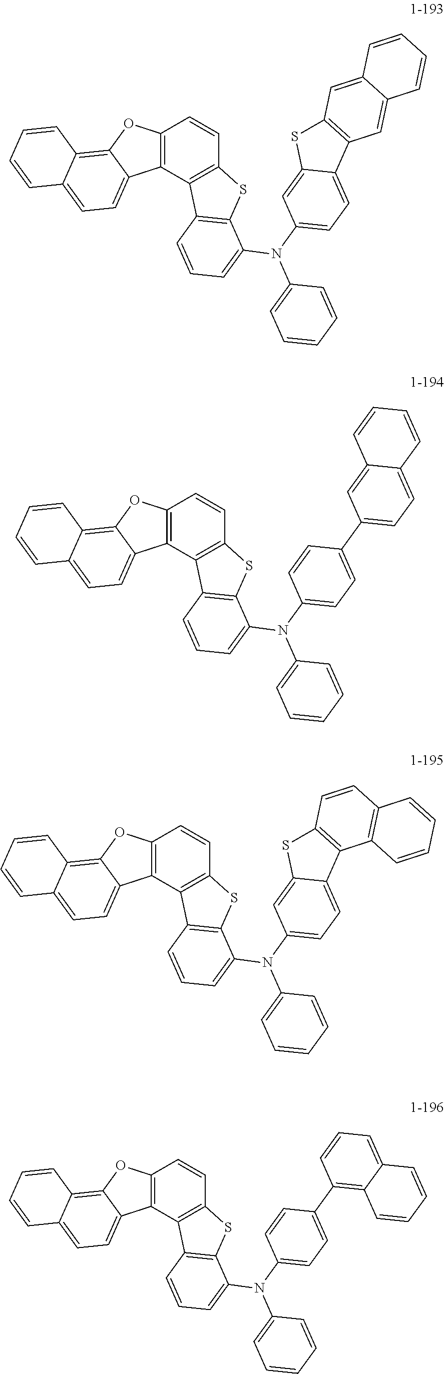 [SMILES: c1ccc(N(c2ccc3c(c2)sc2cc4ccccc4cc23)c2cccc3c2sc2ccc4oc5c6ccccc6ccc5c4c23)cc1 ; c1ccc(N(c2ccc(-c3ccc4ccccc4c3)cc2)c2cccc3c2sc2ccc4oc5c6ccccc6ccc5c4c23)cc1 ; c1ccc(N(c2ccc3c(c2)sc2ccc4ccccc4c23)c2cccc3c2sc2ccc4oc5c6ccccc6ccc5c4c23)cc1 ; c1ccc(N(c2ccc(-c3cccc4ccccc34)cc2)c2cccc3c2sc2ccc4oc5c6ccccc6ccc5c4c23)cc1]

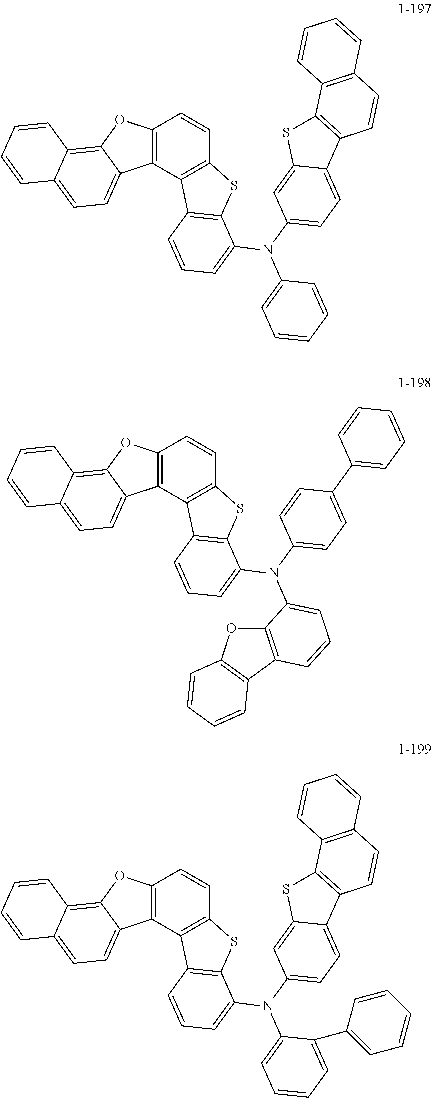 [SMILES: c1ccc(N(c2ccc3c(c2)sc2c4ccccc4ccc32)c2cccc3c2sc2ccc4oc5c6ccccc6ccc5c4c23)cc1 ; c1ccc(-c2ccc(N(c3cccc4c3oc3ccccc34)c3cccc4c3sc3ccc5oc6c7ccccc7ccc6c5c34)cc2)cc1 ; c1ccc(-c2ccccc2N(c2ccc3c(c2)sc2c4ccccc4ccc32)c2cccc3c2sc2ccc4oc5c6ccccc6ccc5c4c23)cc1]

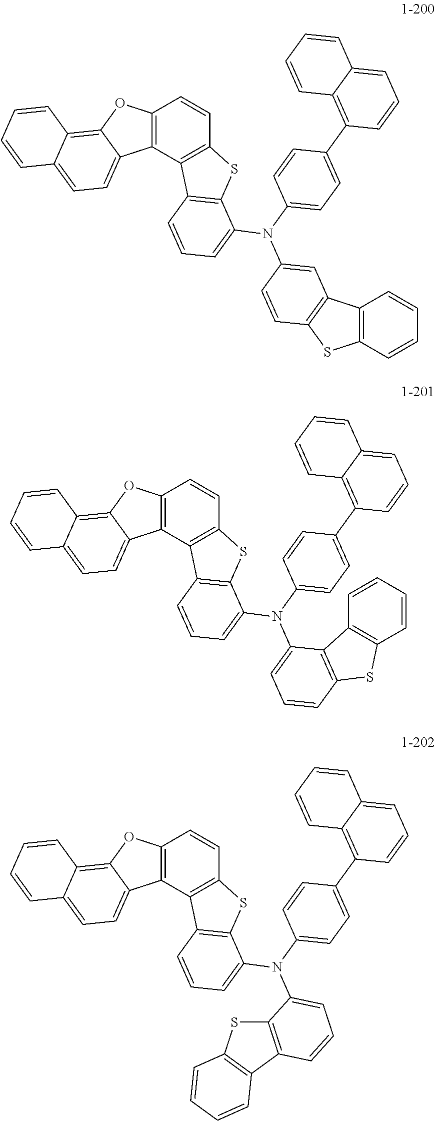 [SMILES: c1ccc2c(-c3ccc(N(c4ccc5sc6ccccc6c5c4)c4cccc5c4sc4ccc6oc7c8ccccc8ccc7c6c45)cc3)cccc2c1 ; c1ccc2c(-c3ccc(N(c4cccc5c4sc4ccc6oc7c8ccccc8ccc7c6c45)c4cccc5sc6ccccc6c45)cc3)cccc2c1 ; c1ccc2c(-c3ccc(N(c4cccc5c4sc4ccccc45)c4cccc5c4sc4ccc6oc7c8ccccc8ccc7c6c45)cc3)cccc2c1]

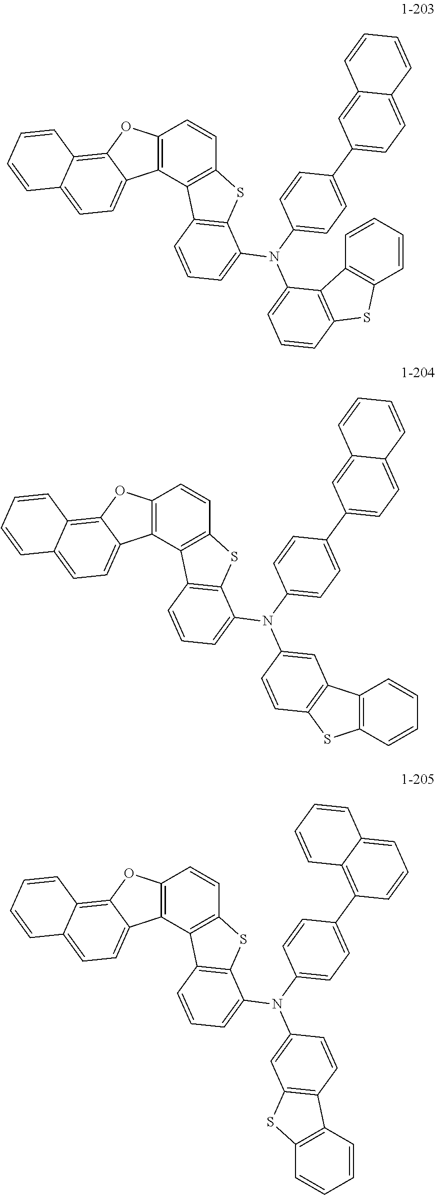 [SMILES: c1ccc2cc(-c3ccc(N(c4cccc5c4sc4ccc6oc7c8ccccc8ccc7c6c45)c4cccc5sc6ccccc6c45)cc3)ccc2c1 ; c1ccc2cc(-c3ccc(N(c4ccc5sc6ccccc6c5c4)c4cccc5c4sc4ccc6oc7c8ccccc8ccc7c6c45)cc3)ccc2c1 ; c1ccc2c(-c3ccc(N(c4ccc5c(c4)sc4ccccc45)c4cccc5c4sc4ccc6oc7c8ccccc8ccc7c6c45)cc3)cccc2c1]

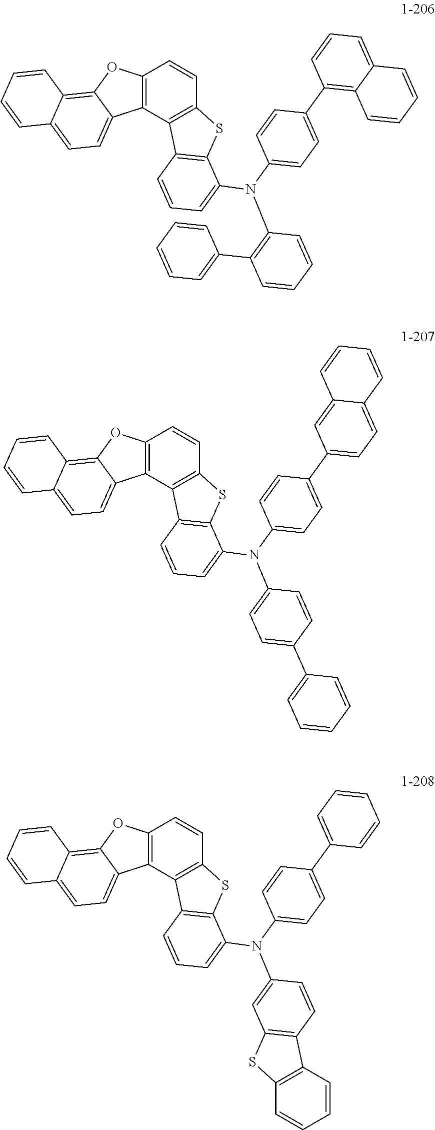 [SMILES: c1ccc(-c2ccccc2N(c2ccc(-c3cccc4ccccc34)cc2)c2cccc3c2sc2ccc4oc5c6ccccc6ccc5c4c23)cc1 ; c1ccc(-c2ccc(N(c3ccc(-c4ccc5ccccc5c4)cc3)c3cccc4c3sc3ccc5oc6c7ccccc7ccc6c5c34)cc2)cc1 ; c1ccc(-c2ccc(N(c3ccc4c(c3)sc3ccccc34)c3cccc4c3sc3ccc5oc6c7ccccc7ccc6c5c34)cc2)cc1]

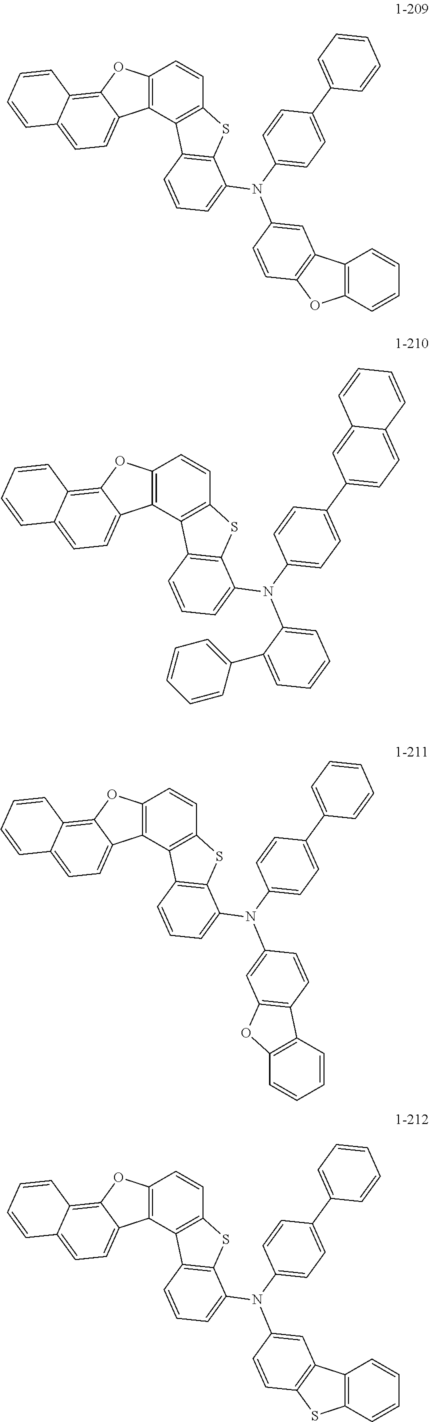 [SMILES: c1ccc(-c2ccc(N(c3ccc4oc5ccccc5c4c3)c3cccc4c3sc3ccc5oc6c7ccccc7ccc6c5c34)cc2)cc1 ; c1ccc(-c2ccccc2N(c2ccc(-c3ccc4ccccc4c3)cc2)c2cccc3c2sc2ccc4oc5c6ccccc6ccc5c4c23)cc1 ; c1ccc(-c2ccc(N(c3ccc4c(c3)oc3ccccc34)c3cccc4c3sc3ccc5oc6c7ccccc7ccc6c5c34)cc2)cc1 ; c1ccc(-c2ccc(N(c3ccc4sc5ccccc5c4c3)c3cccc4c3sc3ccc5oc6c7ccccc7ccc6c5c34)cc2)cc1]

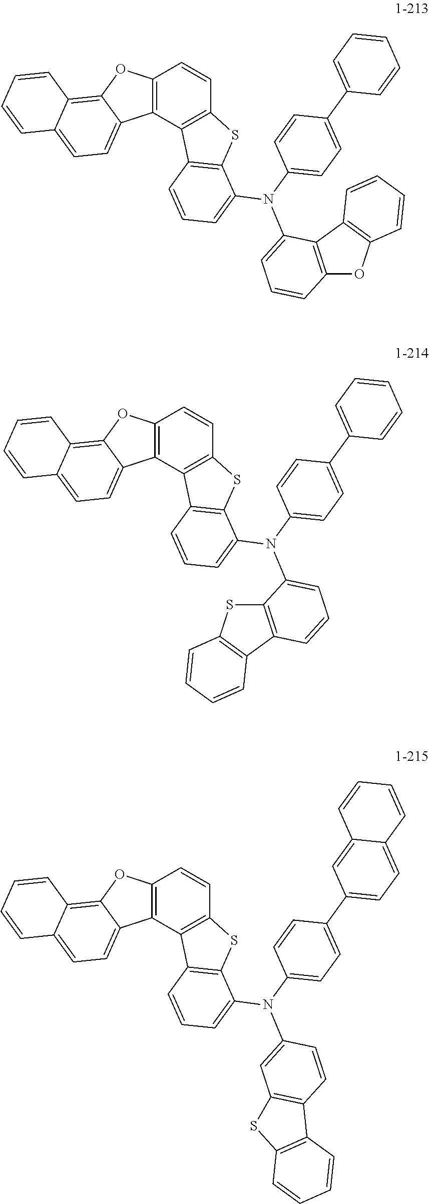 [SMILES: c1ccc(-c2ccc(N(c3cccc4c3sc3ccc5oc6c7ccccc7ccc6c5c34)c3cccc4oc5ccccc5c34)cc2)cc1 ; c1ccc(-c2ccc(N(c3cccc4c3sc3ccccc34)c3cccc4c3sc3ccc5oc6c7ccccc7ccc6c5c34)cc2)cc1 ; c1ccc2cc(-c3ccc(N(c4ccc5c(c4)sc4ccccc45)c4cccc5c4sc4ccc6oc7c8ccccc8ccc7c6c45)cc3)ccc2c1]

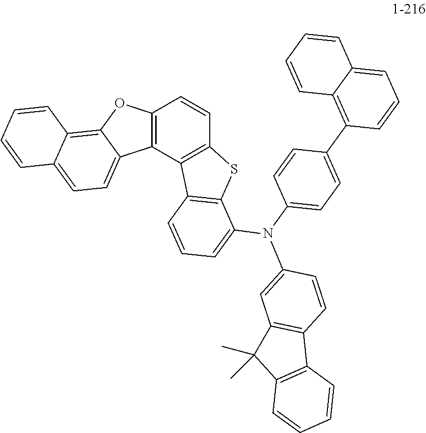 [SMILES: CC1(C)c2ccccc2-c2ccc(N(c3ccc(-c4cccc5ccccc45)cc3)c3cccc4c3sc3ccc5oc6c7ccccc7ccc6c5c34)cc21]

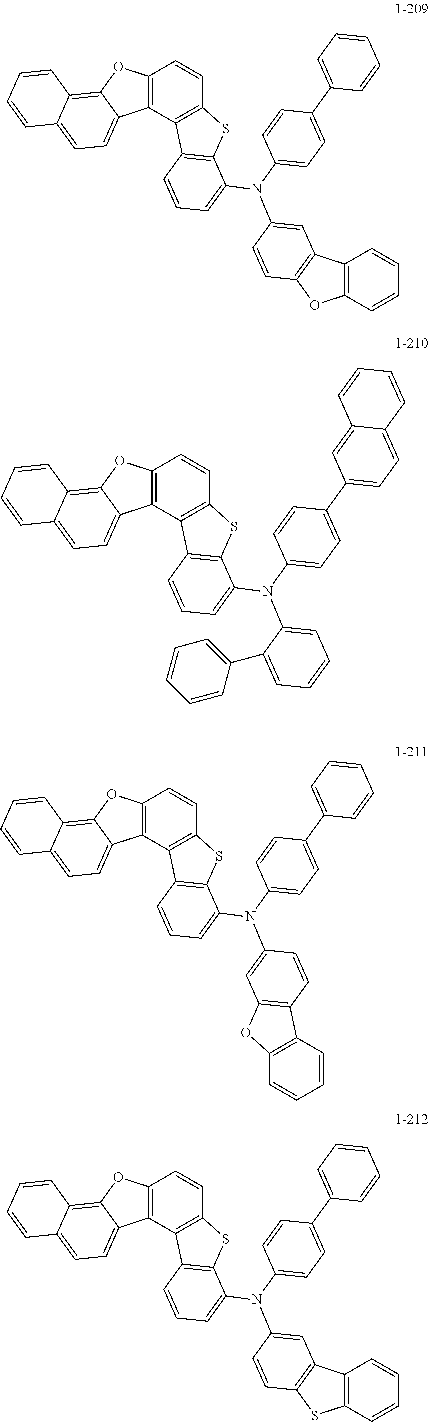 [SMILES: c1ccc(-c2ccc(N(c3ccc4oc5ccccc5c4c3)c3cccc4c3sc3ccc5oc6c7ccccc7ccc6c5c34)cc2)cc1 ; c1ccc(-c2ccccc2N(c2ccc(-c3ccc4ccccc4c3)cc2)c2cccc3c2sc2ccc4oc5c6ccccc6ccc5c4c23)cc1 ; c1ccc(-c2ccc(N(c3ccc4c(c3)oc3ccccc34)c3cccc4c3sc3ccc5oc6c7ccccc7ccc6c5c34)cc2)cc1 ; c1ccc(-c2ccc(N(c3ccc4sc5ccccc5c4c3)c3cccc4c3sc3ccc5oc6c7ccccc7ccc6c5c34)cc2)cc1]

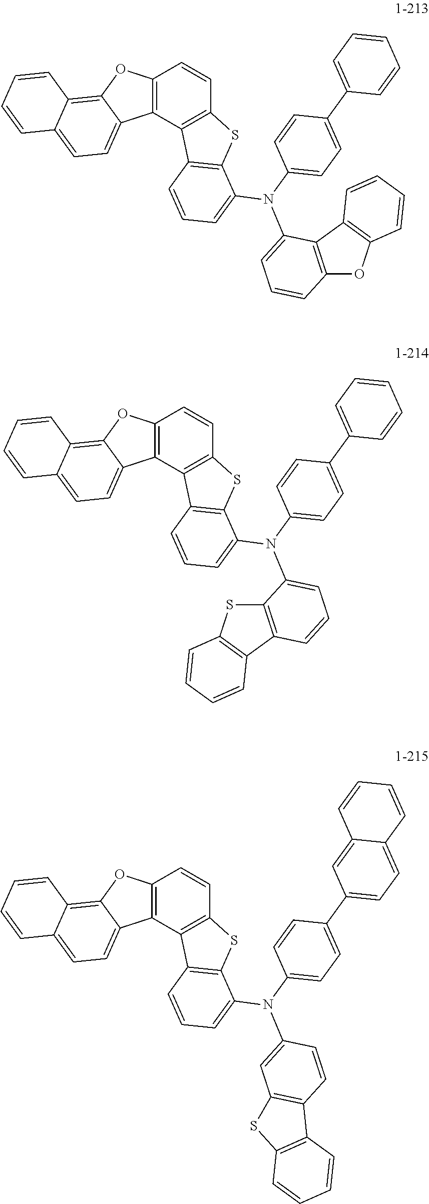 [SMILES: c1ccc(-c2ccc(N(c3cccc4c3sc3ccc5oc6c7ccccc7ccc6c5c34)c3cccc4oc5ccccc5c34)cc2)cc1 ; c1ccc(-c2ccc(N(c3cccc4c3sc3ccccc34)c3cccc4c3sc3ccc5oc6c7ccccc7ccc6c5c34)cc2)cc1 ; c1ccc2cc(-c3ccc(N(c4ccc5c(c4)sc4ccccc45)c4cccc5c4sc4ccc6oc7c8ccccc8ccc7c6c45)cc3)ccc2c1]

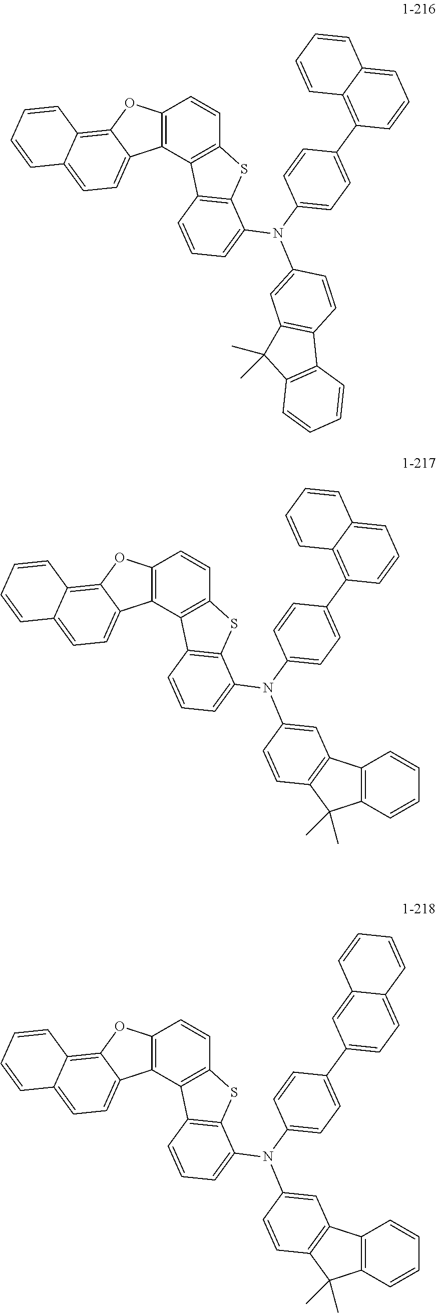 [SMILES: CC1(C)c2ccccc2-c2ccc(N(c3ccc(-c4cccc5ccccc45)cc3)c3cccc4c3sc3ccc5oc6c7ccccc7ccc6c5c34)cc21 ; CC1(C)c2ccccc2-c2cc(N(c3ccc(-c4cccc5ccccc45)cc3)c3cccc4c3sc3ccc5oc6c7ccccc7ccc6c5c34)ccc21 ; CC1(C)c2ccccc2-c2cc(N(c3ccc(-c4ccc5ccccc5c4)cc3)c3cccc4c3sc3ccc5oc6c7ccccc7ccc6c5c34)ccc21]

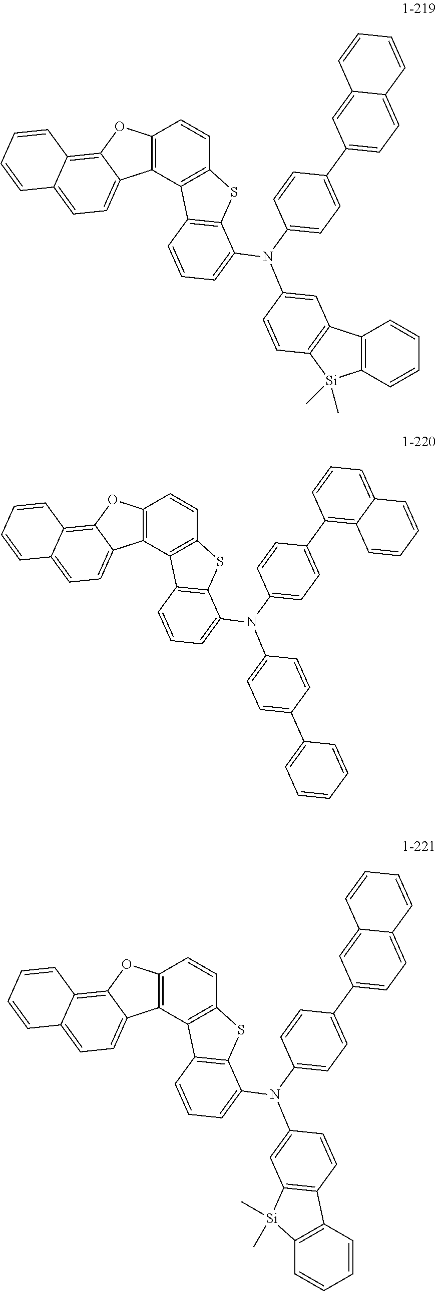 [SMILES: C[Si]1(C)c2ccccc2-c2cc(N(c3ccc(-c4ccc5ccccc5c4)cc3)c3cccc4c3sc3ccc5oc6c7ccccc7ccc6c5c34)ccc21 ; c1ccc(-c2ccc(N(c3ccc(-c4cccc5ccccc45)cc3)c3cccc4c3sc3ccc5oc6c7ccccc7ccc6c5c34)cc2)cc1 ; C[Si]1(C)c2ccccc2-c2ccc(N(c3ccc(-c4ccc5ccccc5c4)cc3)c3cccc4c3sc3ccc5oc6c7ccccc7ccc6c5c34)cc21]

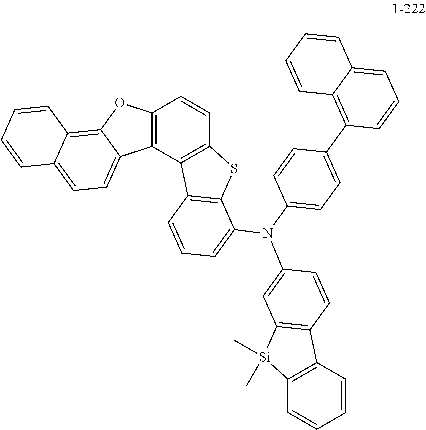 [SMILES: C[Si]1(C)c2ccccc2-c2ccc(N(c3ccc(-c4cccc5ccccc45)cc3)c3cccc4c3sc3ccc5oc6c7ccccc7ccc6c5c34)cc21]

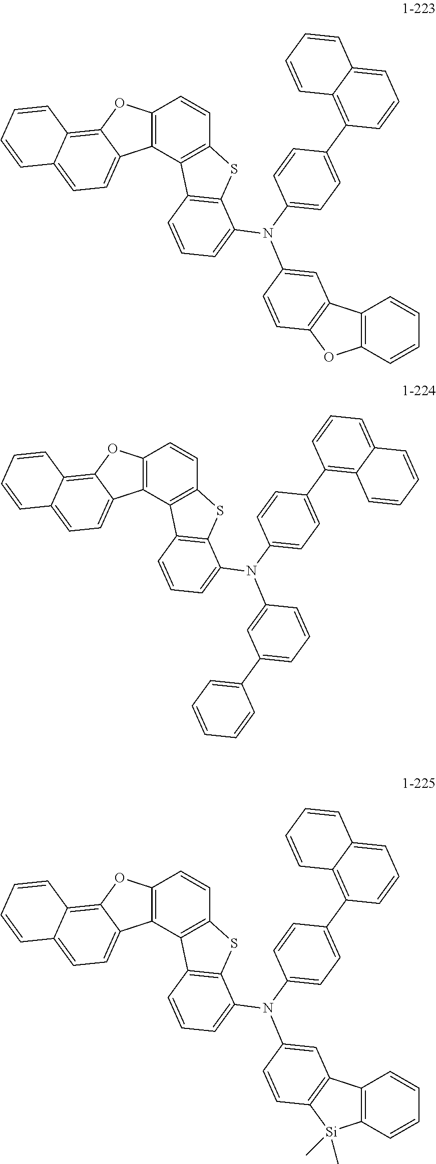 [SMILES: c1ccc2c(-c3ccc(N(c4ccc5oc6ccccc6c5c4)c4cccc5c4sc4ccc6oc7c8ccccc8ccc7c6c45)cc3)cccc2c1 ; c1ccc(-c2cccc(N(c3ccc(-c4cccc5ccccc45)cc3)c3cccc4c3sc3ccc5oc6c7ccccc7ccc6c5c34)c2)cc1 ; C[Si]1(C)c2ccccc2-c2cc(N(c3ccc(-c4cccc5ccccc45)cc3)c3cccc4c3sc3ccc5oc6c7ccccc7ccc6c5c34)ccc21]

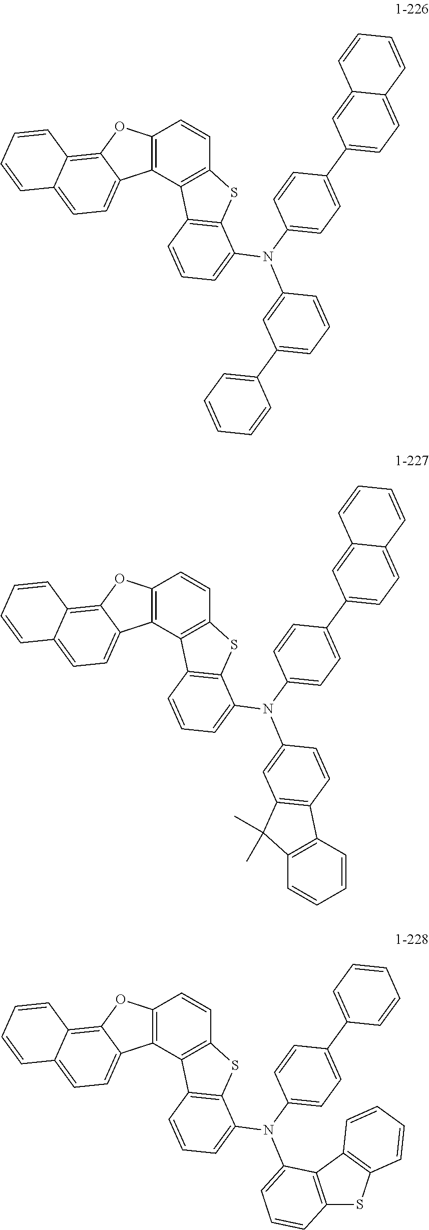 [SMILES: c1ccc(-c2cccc(N(c3ccc(-c4ccc5ccccc5c4)cc3)c3cccc4c3sc3ccc5oc6c7ccccc7ccc6c5c34)c2)cc1 ; CC1(C)c2ccccc2-c2ccc(N(c3ccc(-c4ccc5ccccc5c4)cc3)c3cccc4c3sc3ccc5oc6c7ccccc7ccc6c5c34)cc21 ; c1ccc(-c2ccc(N(c3cccc4c3sc3ccc5oc6c7ccccc7ccc6c5c34)c3cccc4sc5ccccc5c34)cc2)cc1]

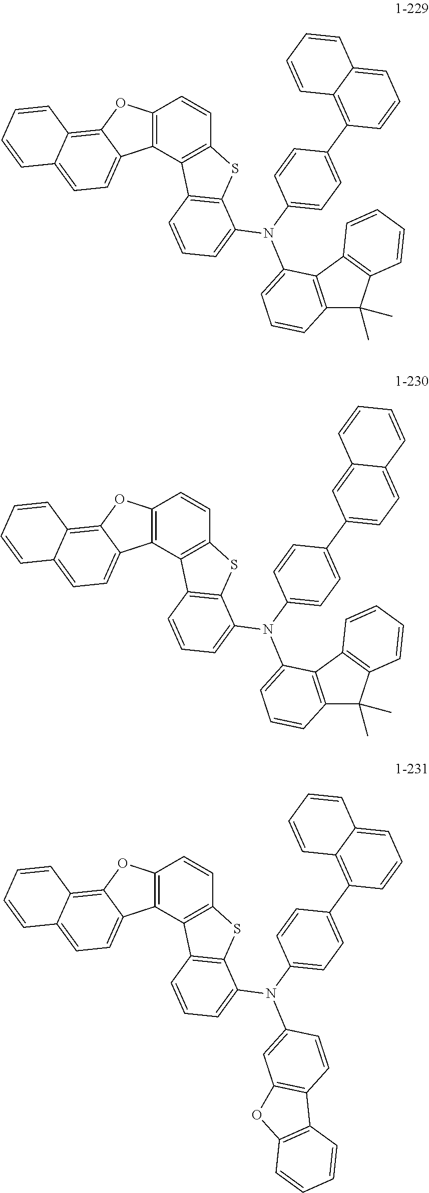 [SMILES: CC1(C)c2ccccc2-c2c(N(c3ccc(-c4cccc5ccccc45)cc3)c3cccc4c3sc3ccc5oc6c7ccccc7ccc6c5c34)cccc21 ; CC1(C)c2ccccc2-c2c(N(c3ccc(-c4ccc5ccccc5c4)cc3)c3cccc4c3sc3ccc5oc6c7ccccc7ccc6c5c34)cccc21 ; c1ccc2c(-c3ccc(N(c4ccc5c(c4)oc4ccccc45)c4cccc5c4sc4ccc6oc7c8ccccc8ccc7c6c45)cc3)cccc2c1]

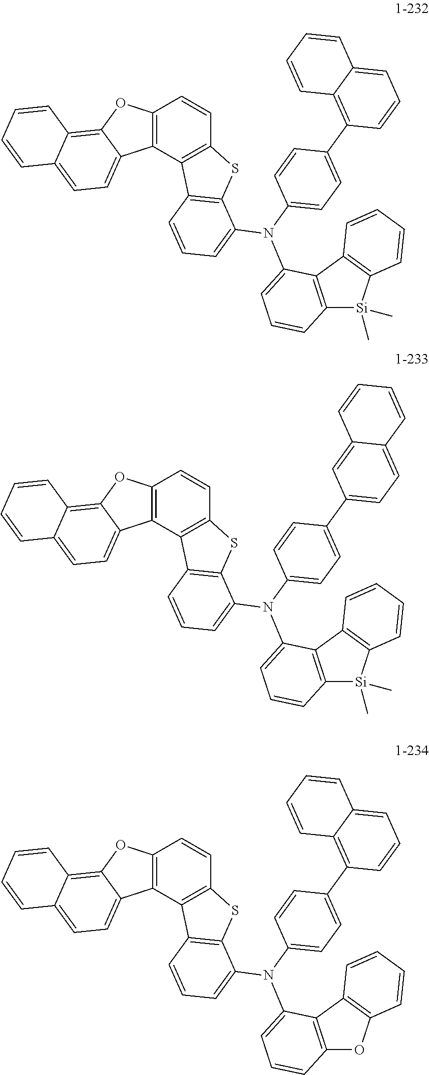

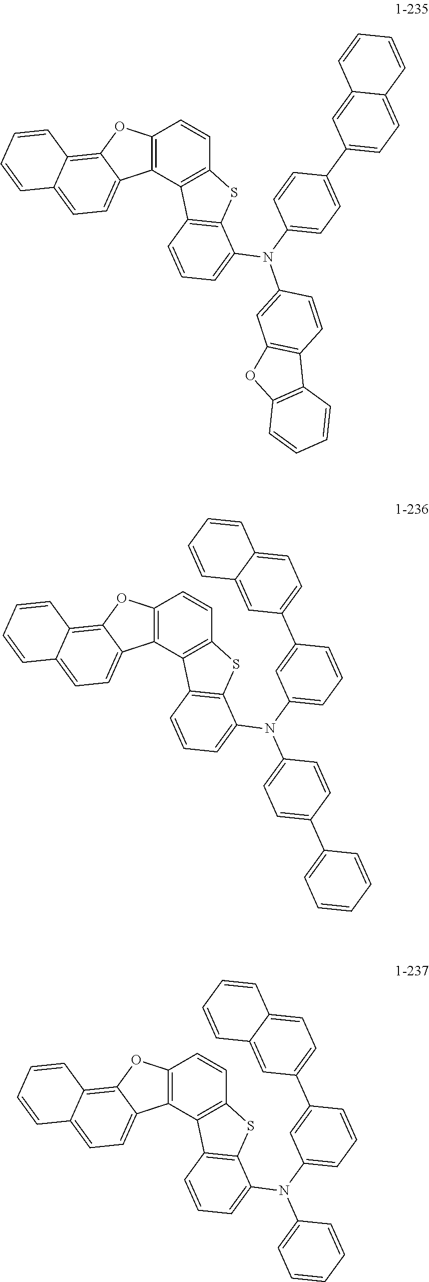 [SMILES: c1ccc2cc(-c3ccc(N(c4ccc5c(c4)oc4ccccc45)c4cccc5c4sc4ccc6oc7c8ccccc8ccc7c6c45)cc3)ccc2c1 ; c1ccc(-c2ccc(N(c3cccc(-c4ccc5ccccc5c4)c3)c3cccc4c3sc3ccc5oc6c7ccccc7ccc6c5c34)cc2)cc1 ; c1ccc(N(c2cccc(-c3ccc4ccccc4c3)c2)c2cccc3c2sc2ccc4oc5c6ccccc6ccc5c4c23)cc1]

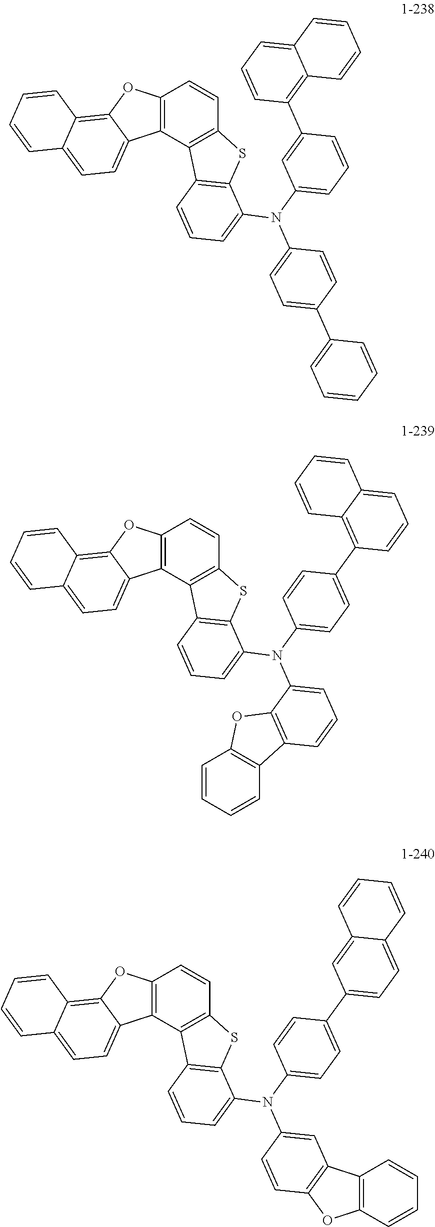 [SMILES: c1ccc(-c2ccc(N(c3cccc(-c4cccc5ccccc45)c3)c3cccc4c3sc3ccc5oc6c7ccccc7ccc6c5c34)cc2)cc1 ; c1ccc2c(-c3ccc(N(c4cccc5c4oc4ccccc45)c4cccc5c4sc4ccc6oc7c8ccccc8ccc7c6c45)cc3)cccc2c1 ; c1ccc2cc(-c3ccc(N(c4ccc5oc6ccccc6c5c4)c4cccc5c4sc4ccc6oc7c8ccccc8ccc7c6c45)cc3)ccc2c1]

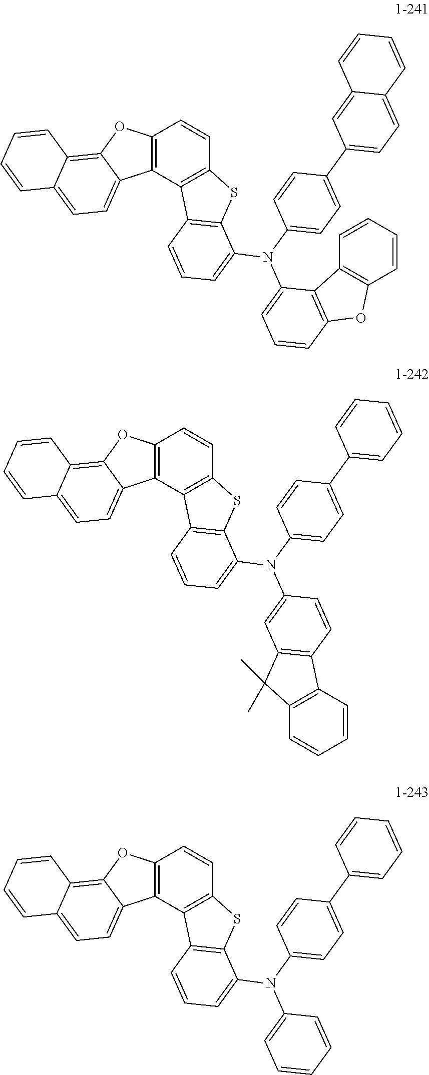 [SMILES: c1ccc2cc(-c3ccc(N(c4cccc5c4sc4ccc6oc7c8ccccc8ccc7c6c45)c4cccc5oc6ccccc6c45)cc3)ccc2c1 ; CC1(C)c2ccccc2-c2ccc(N(c3ccc(-c4ccccc4)cc3)c3cccc4c3sc3ccc5oc6c7ccccc7ccc6c5c34)cc21 ; c1ccc(-c2ccc(N(c3ccccc3)c3cccc4c3sc3ccc5oc6c7ccccc7ccc6c5c34)cc2)cc1]

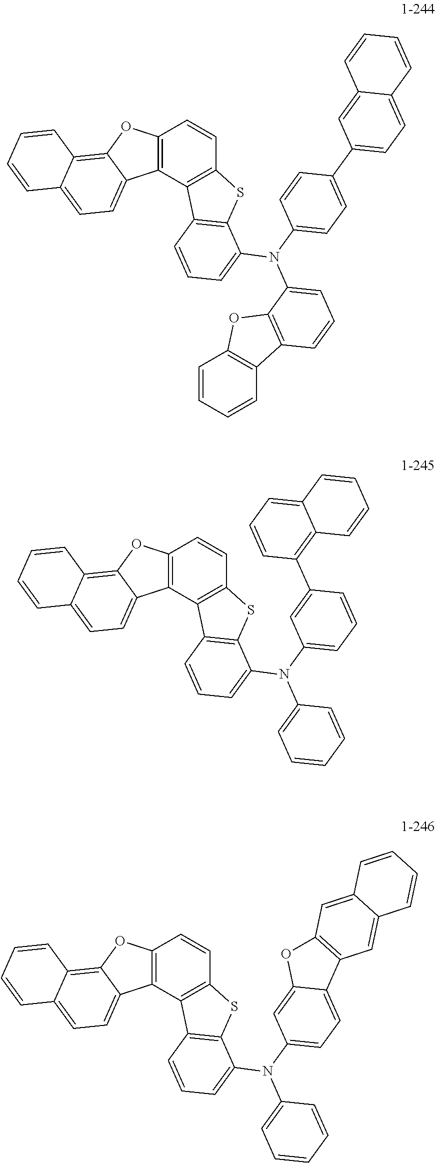 [SMILES: c1ccc2cc(-c3ccc(N(c4cccc5c4oc4ccccc45)c4cccc5c4sc4ccc6oc7c8ccccc8ccc7c6c45)cc3)ccc2c1 ; c1ccc(N(c2cccc(-c3cccc4ccccc34)c2)c2cccc3c2sc2ccc4oc5c6ccccc6ccc5c4c23)cc1 ; c1ccc(N(c2ccc3c(c2)oc2cc4ccccc4cc23)c2cccc3c2sc2ccc4oc5c6ccccc6ccc5c4c23)cc1]

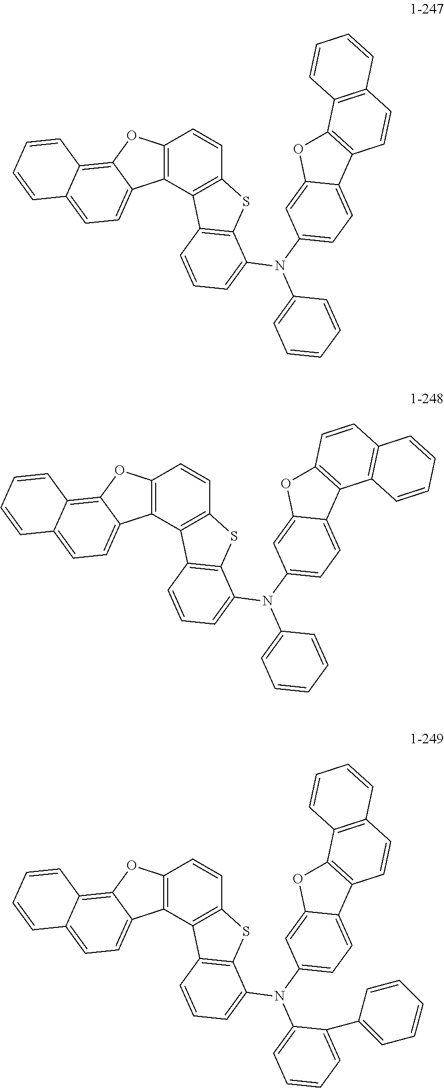 [SMILES: c1ccc(N(c2ccc3c(c2)oc2c4ccccc4ccc32)c2cccc3c2sc2ccc4oc5c6ccccc6ccc5c4c23)cc1 ; c1ccc(N(c2ccc3c(c2)oc2ccc4ccccc4c23)c2cccc3c2sc2ccc4oc5c6ccccc6ccc5c4c23)cc1 ; c1ccc(-c2ccccc2N(c2ccc3c(c2)oc2c4ccccc4ccc32)c2cccc3c2sc2ccc4oc5c6ccccc6ccc5c4c23)cc1]

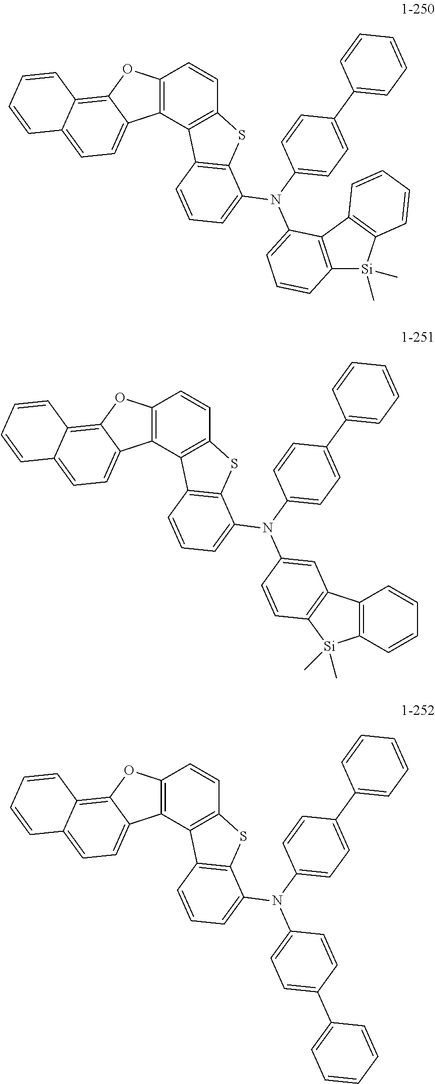 [SMILES: C[Si]1(C)c2ccccc2-c2c(N(c3ccc(-c4ccccc4)cc3)c3cccc4c3sc3ccc5oc6c7ccccc7ccc6c5c34)cccc21 ; C[Si]1(C)c2ccccc2-c2cc(N(c3ccc(-c4ccccc4)cc3)c3cccc4c3sc3ccc5oc6c7ccccc7ccc6c5c34)ccc21 ; c1ccc(-c2ccc(N(c3ccc(-c4ccccc4)cc3)c3cccc4c3sc3ccc5oc6c7ccccc7ccc6c5c34)cc2)cc1]

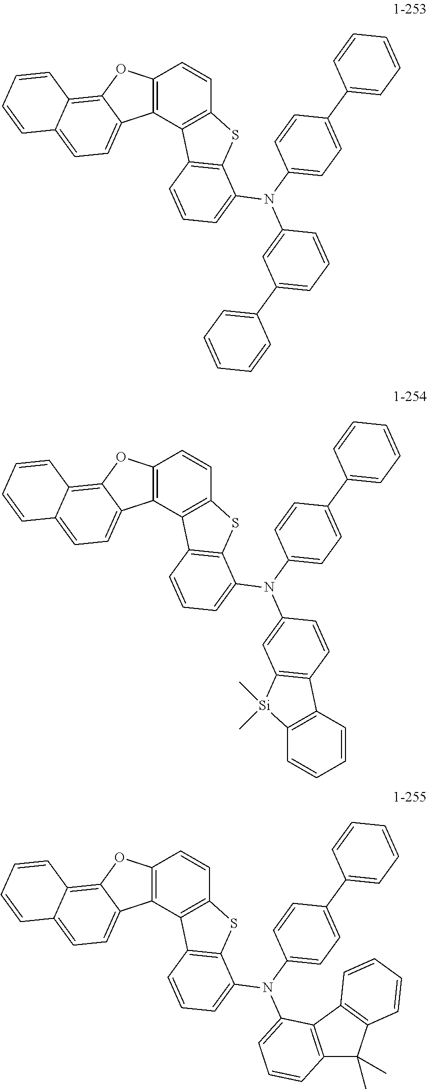 [SMILES: c1ccc(-c2ccc(N(c3cccc(-c4ccccc4)c3)c3cccc4c3sc3ccc5oc6c7ccccc7ccc6c5c34)cc2)cc1 ; C[Si]1(C)c2ccccc2-c2ccc(N(c3ccc(-c4ccccc4)cc3)c3cccc4c3sc3ccc5oc6c7ccccc7ccc6c5c34)cc21 ; CC1(C)c2ccccc2-c2c(N(c3ccc(-c4ccccc4)cc3)c3cccc4c3sc3ccc5oc6c7ccccc7ccc6c5c34)cccc21]

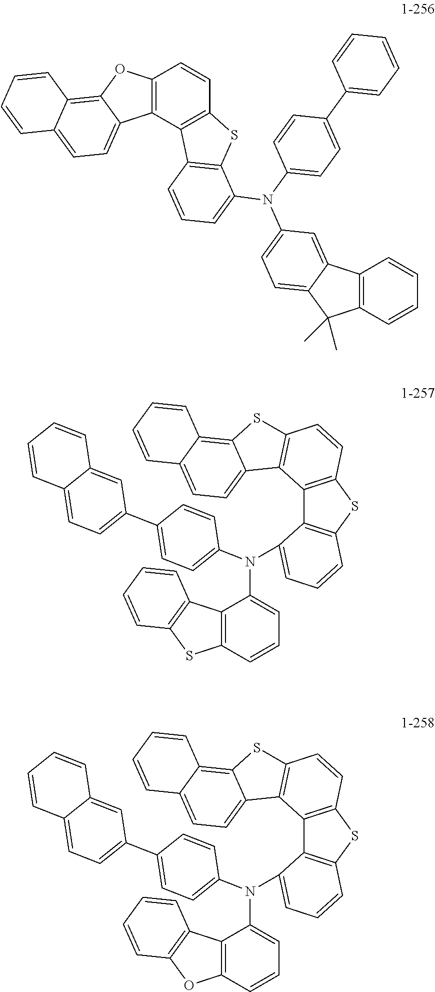 [SMILES: CC1(C)c2ccccc2-c2cc(N(c3ccc(-c4ccccc4)cc3)c3cccc4c3sc3ccc5oc6c7ccccc7ccc6c5c34)ccc21 ; c1ccc2cc(-c3ccc(N(c4cccc5sc6ccccc6c45)c4cccc5sc6ccc7sc8c9ccccc9ccc8c7c6c45)cc3)ccc2c1 ; c1ccc2cc(-c3ccc(N(c4cccc5oc6ccccc6c45)c4cccc5sc6ccc7sc8c9ccccc9ccc8c7c6c45)cc3)ccc2c1]

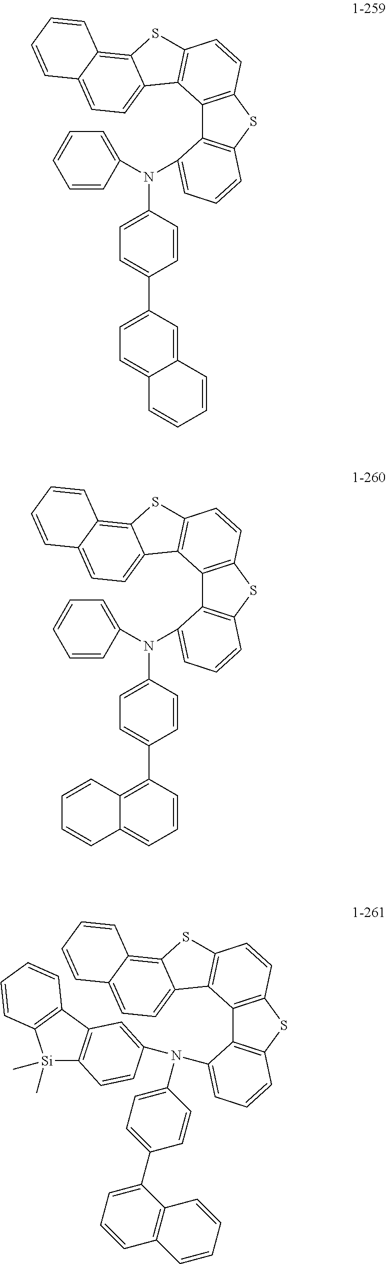 [SMILES: c1ccc(N(c2ccc(-c3ccc4ccccc4c3)cc2)c2cccc3sc4ccc5sc6c7ccccc7ccc6c5c4c23)cc1 ; c1ccc(N(c2ccc(-c3cccc4ccccc34)cc2)c2cccc3sc4ccc5sc6c7ccccc7ccc6c5c4c23)cc1 ; C[Si]1(C)c2ccccc2-c2cc(N(c3ccc(-c4cccc5ccccc45)cc3)c3cccc4sc5ccc6sc7c8ccccc8ccc7c6c5c34)ccc21]

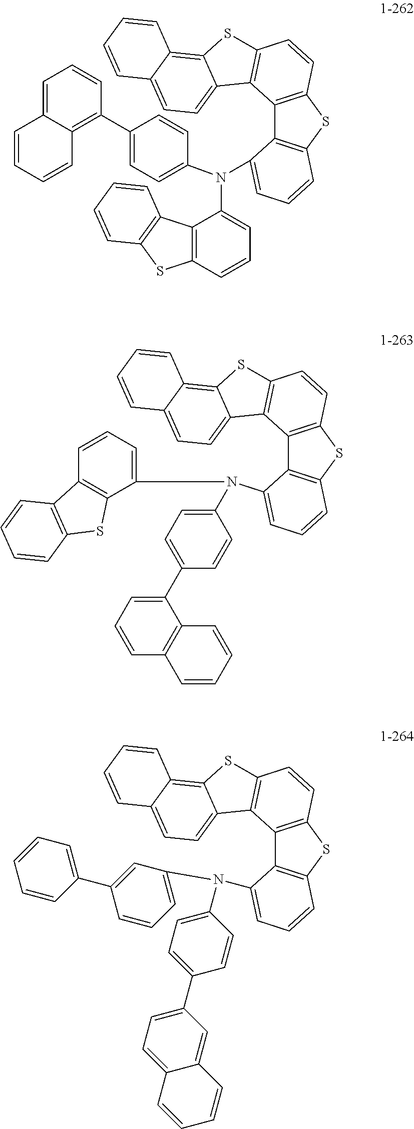 [SMILES: c1ccc2c(-c3ccc(N(c4cccc5sc6ccccc6c45)c4cccc5sc6ccc7sc8c9ccccc9ccc8c7c6c45)cc3)cccc2c1 ; c1ccc2c(-c3ccc(N(c4cccc5c4sc4ccccc45)c4cccc5sc6ccc7sc8c9ccccc9ccc8c7c6c45)cc3)cccc2c1 ; c1ccc(-c2cccc(N(c3ccc(-c4ccc5ccccc5c4)cc3)c3cccc4sc5ccc6sc7c8ccccc8ccc7c6c5c34)c2)cc1]

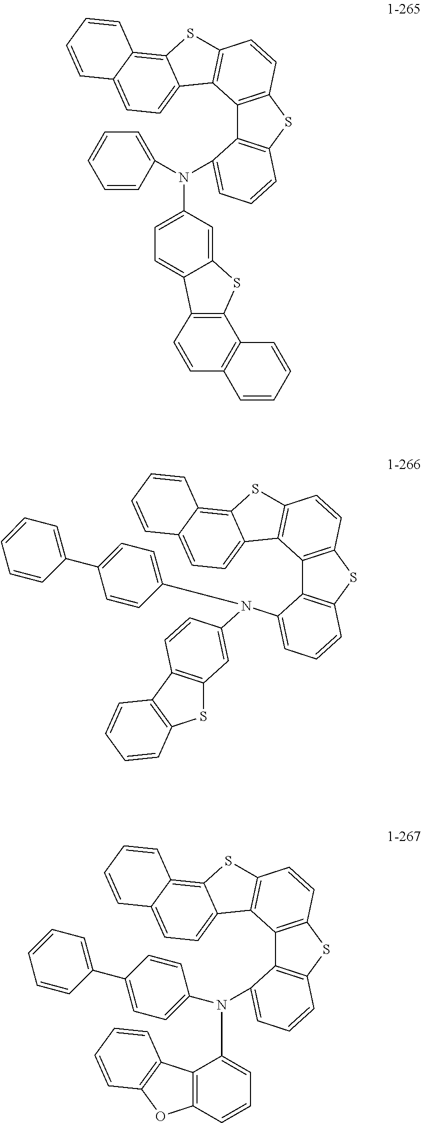 [SMILES: c1ccc(N(c2ccc3c(c2)sc2c4ccccc4ccc32)c2cccc3sc4ccc5sc6c7ccccc7ccc6c5c4c23)cc1 ; c1ccc(-c2ccc(N(c3ccc4c(c3)sc3ccccc34)c3cccc4sc5ccc6sc7c8ccccc8ccc7c6c5c34)cc2)cc1 ; c1ccc(-c2ccc(N(c3cccc4oc5ccccc5c34)c3cccc4sc5ccc6sc7c8ccccc8ccc7c6c5c34)cc2)cc1]

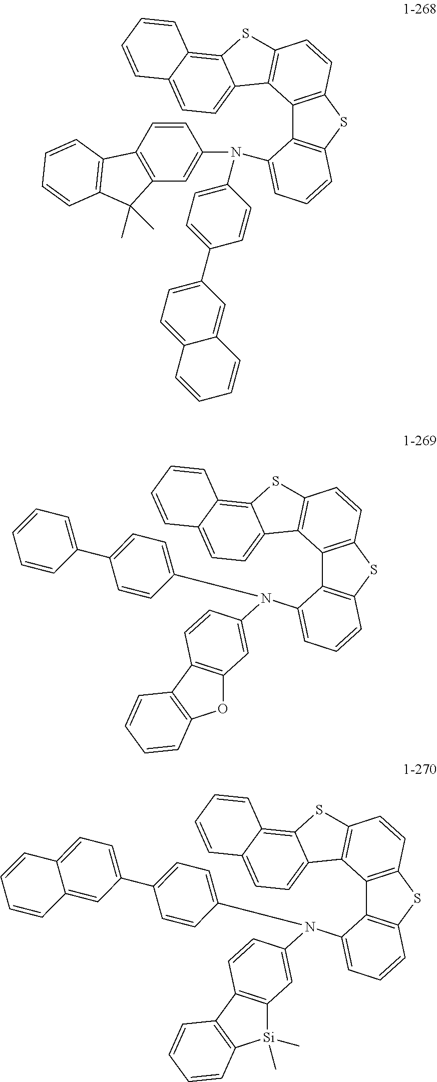 [SMILES: CC1(C)c2ccccc2-c2ccc(N(c3ccc(-c4ccc5ccccc5c4)cc3)c3cccc4sc5ccc6sc7c8ccccc8ccc7c6c5c34)cc21 ; c1ccc(-c2ccc(N(c3ccc4c(c3)oc3ccccc34)c3cccc4sc5ccc6sc7c8ccccc8ccc7c6c5c34)cc2)cc1 ; C[Si]1(C)c2ccccc2-c2ccc(N(c3ccc(-c4ccc5ccccc5c4)cc3)c3cccc4sc5ccc6sc7c8ccccc8ccc7c6c5c34)cc21]

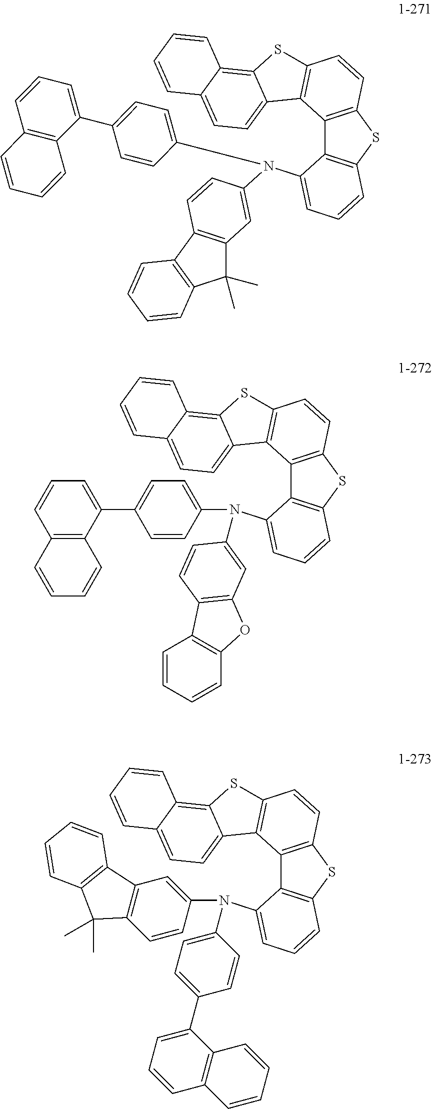 [SMILES: CC1(C)c2ccccc2-c2ccc(N(c3ccc(-c4cccc5ccccc45)cc3)c3cccc4sc5ccc6sc7c8ccccc8ccc7c6c5c34)cc21 ; c1ccc2c(-c3ccc(N(c4ccc5c(c4)oc4ccccc45)c4cccc5sc6ccc7sc8c9ccccc9ccc8c7c6c45)cc3)cccc2c1 ; CC1(C)c2ccccc2-c2cc(N(c3ccc(-c4cccc5ccccc45)cc3)c3cccc4sc5ccc6sc7c8ccccc8ccc7c6c5c34)ccc21]

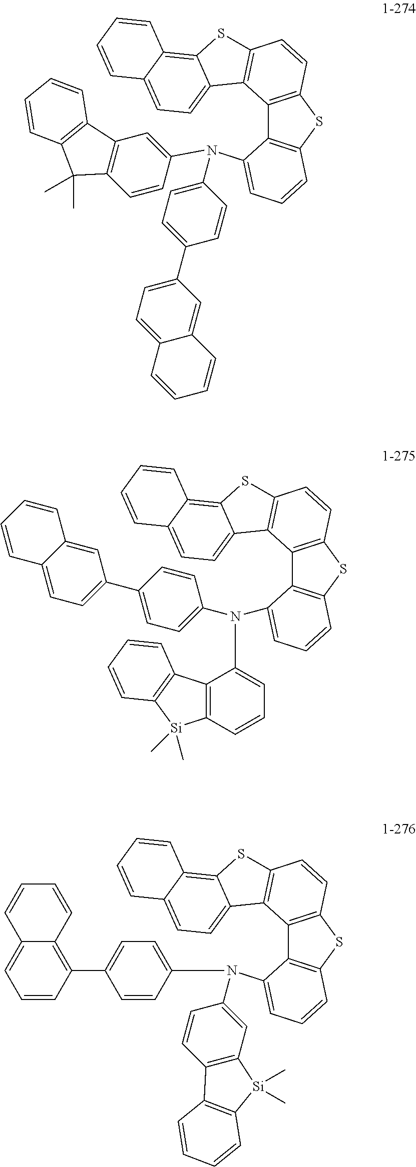 [SMILES: CC1(C)c2ccccc2-c2cc(N(c3ccc(-c4ccc5ccccc5c4)cc3)c3cccc4sc5ccc6sc7c8ccccc8ccc7c6c5c34)ccc21 ; C[Si]1(C)c2ccccc2-c2c(N(c3ccc(-c4ccc5ccccc5c4)cc3)c3cccc4sc5ccc6sc7c8ccccc8ccc7c6c5c34)cccc21 ; C[Si]1(C)c2ccccc2-c2ccc(N(c3ccc(-c4cccc5ccccc45)cc3)c3cccc4sc5ccc6sc7c8ccccc8ccc7c6c5c34)cc21]

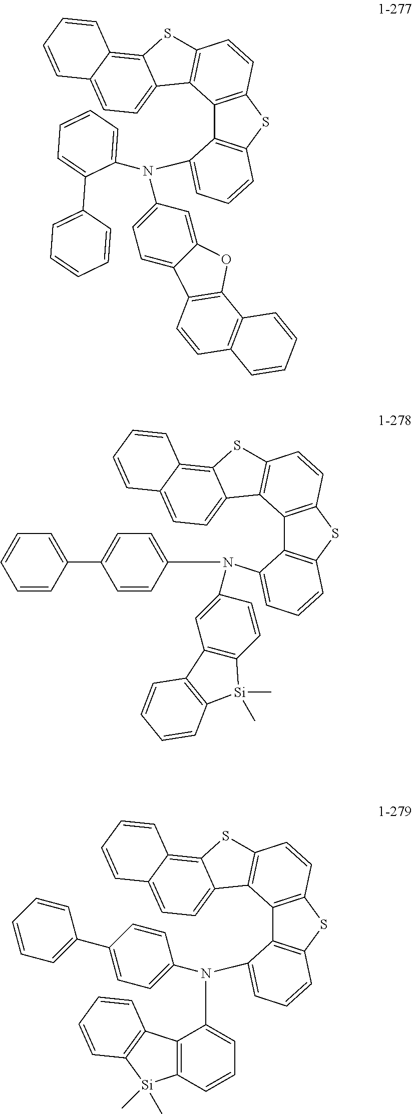 [SMILES: c1ccc(-c2ccccc2N(c2ccc3c(c2)oc2c4ccccc4ccc32)c2cccc3sc4ccc5sc6c7ccccc7ccc6c5c4c23)cc1 ; C[Si]1(C)c2ccccc2-c2cc(N(c3ccc(-c4ccccc4)cc3)c3cccc4sc5ccc6sc7c8ccccc8ccc7c6c5c34)ccc21 ; C[Si]1(C)c2ccccc2-c2c(N(c3ccc(-c4ccccc4)cc3)c3cccc4sc5ccc6sc7c8ccccc8ccc7c6c5c34)cccc21]

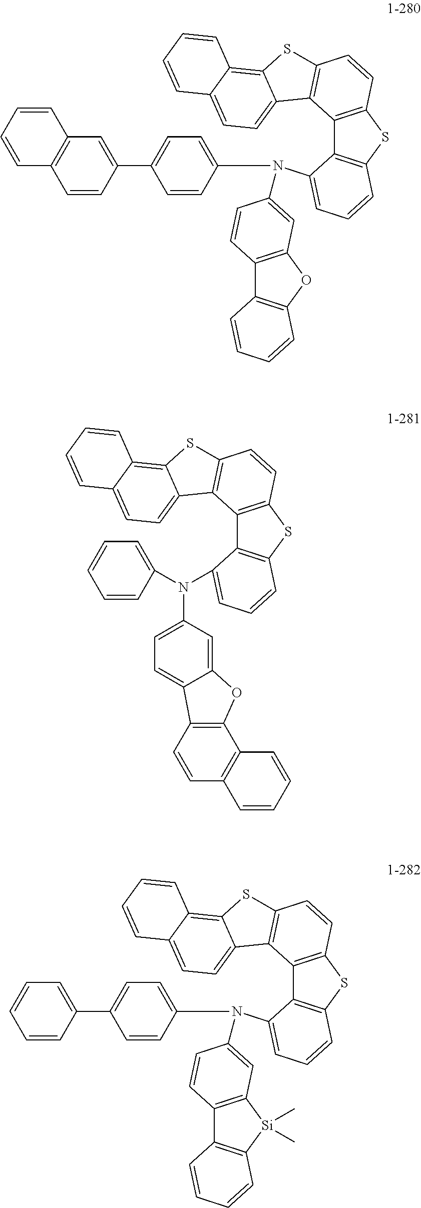 [SMILES: c1ccc2cc(-c3ccc(N(c4ccc5c(c4)oc4ccccc45)c4cccc5sc6ccc7sc8c9ccccc9ccc8c7c6c45)cc3)ccc2c1 ; c1ccc(N(c2ccc3c(c2)oc2c4ccccc4ccc32)c2cccc3sc4ccc5sc6c7ccccc7ccc6c5c4c23)cc1 ; C[Si]1(C)c2ccccc2-c2ccc(N(c3ccc(-c4ccccc4)cc3)c3cccc4sc5ccc6sc7c8ccccc8ccc7c6c5c34)cc21]

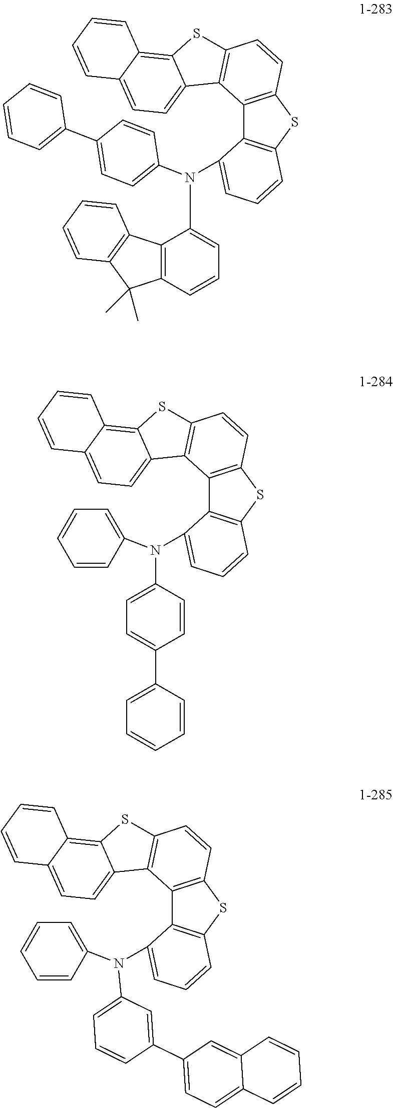 [SMILES: CC1(C)c2ccccc2-c2c(N(c3ccc(-c4ccccc4)cc3)c3cccc4sc5ccc6sc7c8ccccc8ccc7c6c5c34)cccc21 ; c1ccc(-c2ccc(N(c3ccccc3)c3cccc4sc5ccc6sc7c8ccccc8ccc7c6c5c34)cc2)cc1 ; c1ccc(N(c2cccc(-c3ccc4ccccc4c3)c2)c2cccc3sc4ccc5sc6c7ccccc7ccc6c5c4c23)cc1]

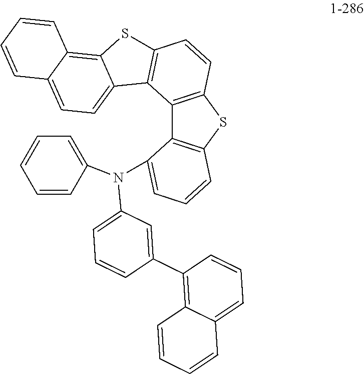 [SMILES: c1ccc(N(c2cccc(-c3cccc4ccccc34)c2)c2cccc3sc4ccc5sc6c7ccccc7ccc6c5c4c23)cc1]

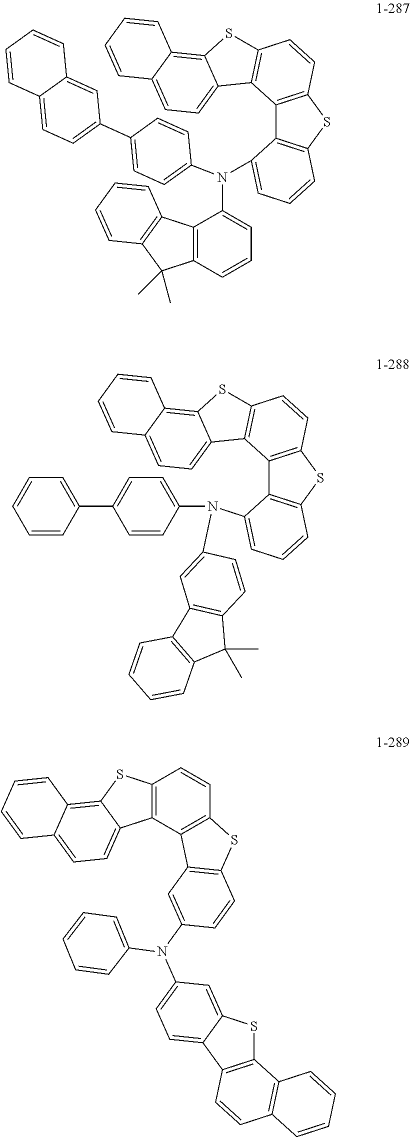 [SMILES: CC1(C)c2ccccc2-c2c(N(c3ccc(-c4ccc5ccccc5c4)cc3)c3cccc4sc5ccc6sc7c8ccccc8ccc7c6c5c34)cccc21 ; CC1(C)c2ccccc2-c2cc(N(c3ccc(-c4ccccc4)cc3)c3cccc4sc5ccc6sc7c8ccccc8ccc7c6c5c34)ccc21 ; c1ccc(N(c2ccc3c(c2)sc2c4ccccc4ccc32)c2ccc3sc4ccc5sc6c7ccccc7ccc6c5c4c3c2)cc1]

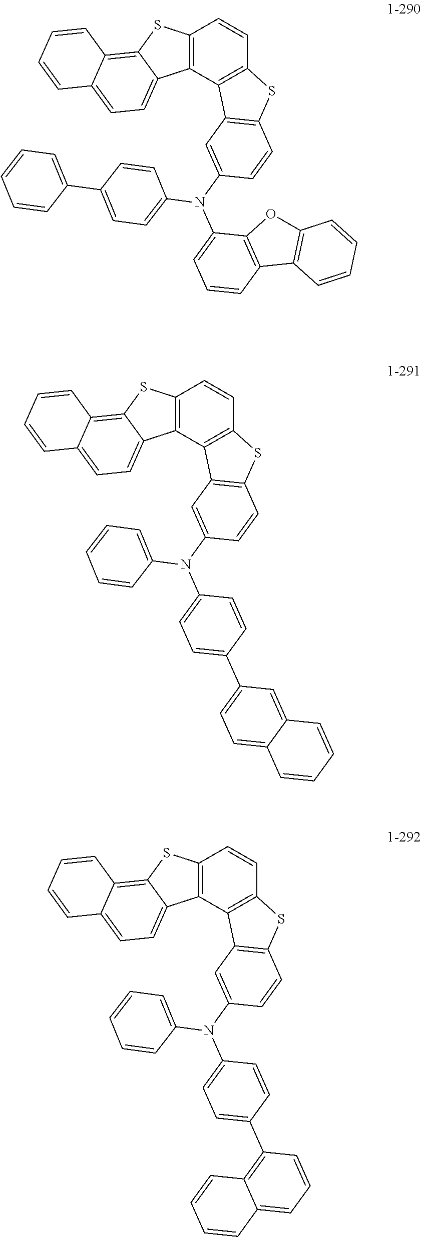 [SMILES: c1ccc(-c2ccc(N(c3ccc4sc5ccc6sc7c8ccccc8ccc7c6c5c4c3)c3cccc4c3oc3ccccc34)cc2)cc1 ; c1ccc(N(c2ccc(-c3ccc4ccccc4c3)cc2)c2ccc3sc4ccc5sc6c7ccccc7ccc6c5c4c3c2)cc1 ; c1ccc(N(c2ccc(-c3cccc4ccccc34)cc2)c2ccc3sc4ccc5sc6c7ccccc7ccc6c5c4c3c2)cc1]

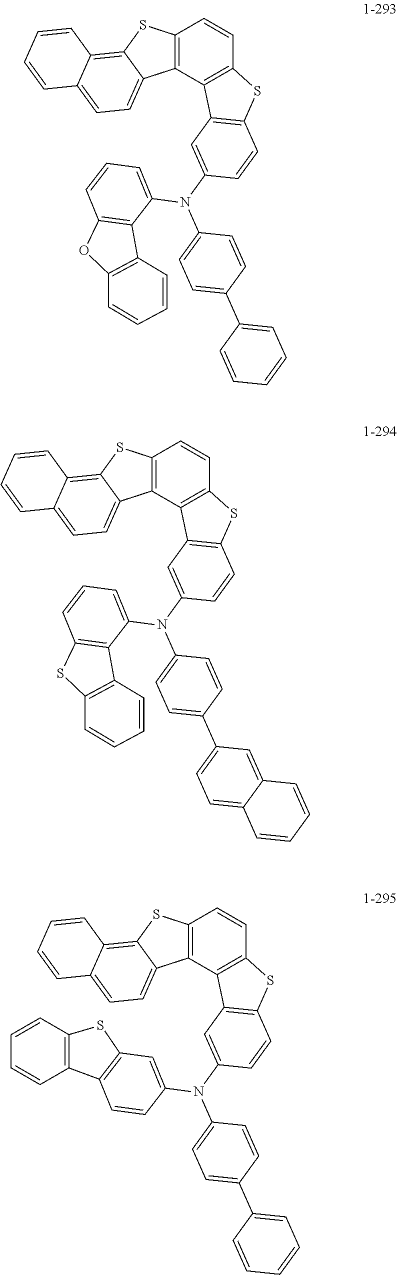 [SMILES: c1ccc(-c2ccc(N(c3ccc4sc5ccc6sc7c8ccccc8ccc7c6c5c4c3)c3cccc4oc5ccccc5c34)cc2)cc1 ; c1ccc2cc(-c3ccc(N(c4ccc5sc6ccc7sc8c9ccccc9ccc8c7c6c5c4)c4cccc5sc6ccccc6c45)cc3)ccc2c1 ; c1ccc(-c2ccc(N(c3ccc4c(c3)sc3ccccc34)c3ccc4sc5ccc6sc7c8ccccc8ccc7c6c5c4c3)cc2)cc1]

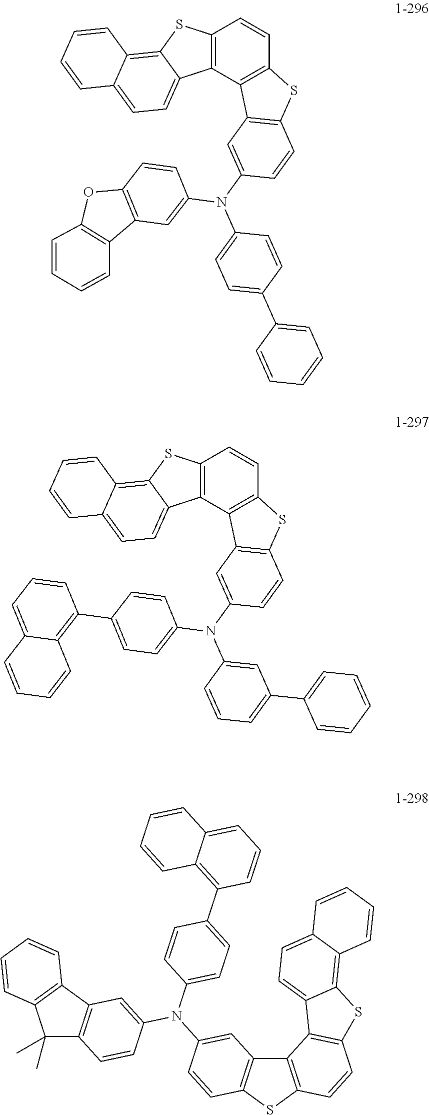 [SMILES: c1ccc(-c2ccc(N(c3ccc4oc5ccccc5c4c3)c3ccc4sc5ccc6sc7c8ccccc8ccc7c6c5c4c3)cc2)cc1 ; c1ccc(-c2cccc(N(c3ccc(-c4cccc5ccccc45)cc3)c3ccc4sc5ccc6sc7c8ccccc8ccc7c6c5c4c3)c2)cc1 ; CC1(C)c2ccccc2-c2cc(N(c3ccc(-c4cccc5ccccc45)cc3)c3ccc4sc5ccc6sc7c8ccccc8ccc7c6c5c4c3)ccc21]

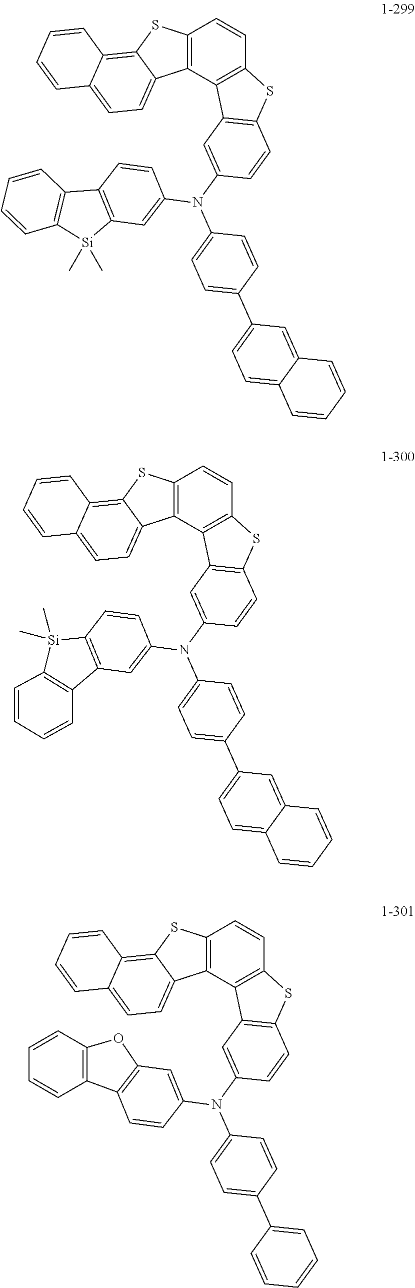 [SMILES: C[Si]1(C)c2ccccc2-c2ccc(N(c3ccc(-c4ccc5ccccc5c4)cc3)c3ccc4sc5ccc6sc7c8ccccc8ccc7c6c5c4c3)cc21 ; C[Si]1(C)c2ccccc2-c2cc(N(c3ccc(-c4ccc5ccccc5c4)cc3)c3ccc4sc5ccc6sc7c8ccccc8ccc7c6c5c4c3)ccc21 ; c1ccc(-c2ccc(N(c3ccc4c(c3)oc3ccccc34)c3ccc4sc5ccc6sc7c8ccccc8ccc7c6c5c4c3)cc2)cc1]

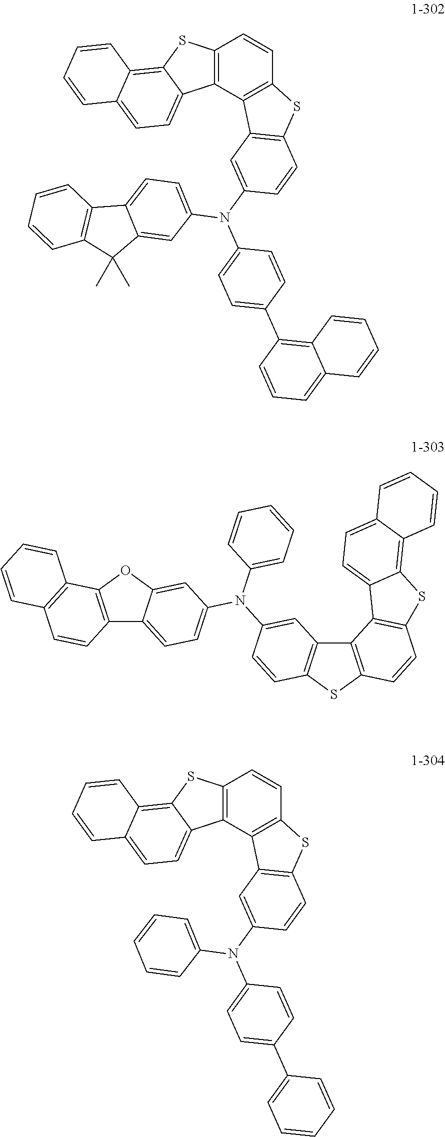 [SMILES: CC1(C)c2ccccc2-c2ccc(N(c3ccc(-c4cccc5ccccc45)cc3)c3ccc4sc5ccc6sc7c8ccccc8ccc7c6c5c4c3)cc21 ; c1ccc(N(c2ccc3c(c2)oc2c4ccccc4ccc32)c2ccc3sc4ccc5sc6c7ccccc7ccc6c5c4c3c2)cc1 ; c1ccc(-c2ccc(N(c3ccccc3)c3ccc4sc5ccc6sc7c8ccccc8ccc7c6c5c4c3)cc2)cc1]

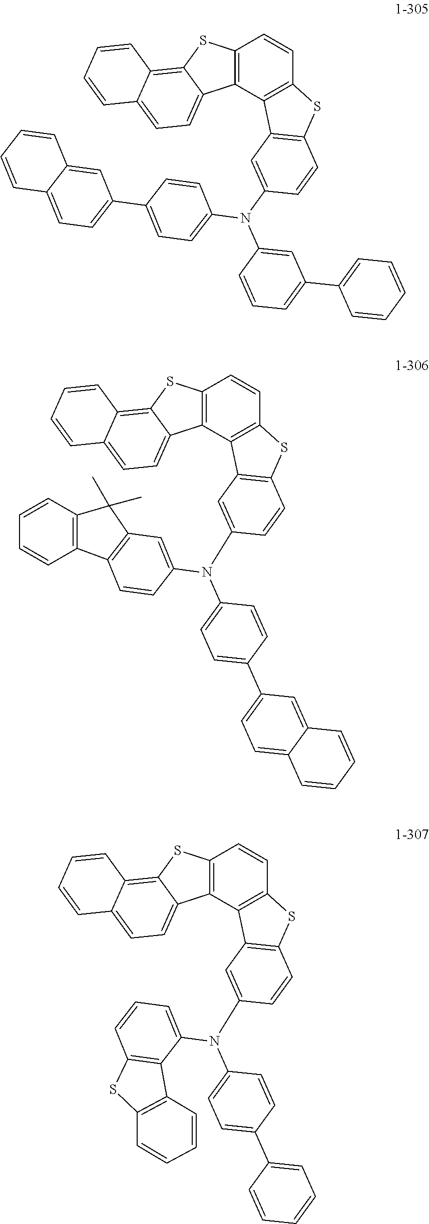 [SMILES: c1ccc(-c2cccc(N(c3ccc(-c4ccc5ccccc5c4)cc3)c3ccc4sc5ccc6sc7c8ccccc8ccc7c6c5c4c3)c2)cc1 ; CC1(C)c2ccccc2-c2ccc(N(c3ccc(-c4ccc5ccccc5c4)cc3)c3ccc4sc5ccc6sc7c8ccccc8ccc7c6c5c4c3)cc21 ; c1ccc(-c2ccc(N(c3ccc4sc5ccc6sc7c8ccccc8ccc7c6c5c4c3)c3cccc4sc5ccccc5c34)cc2)cc1]

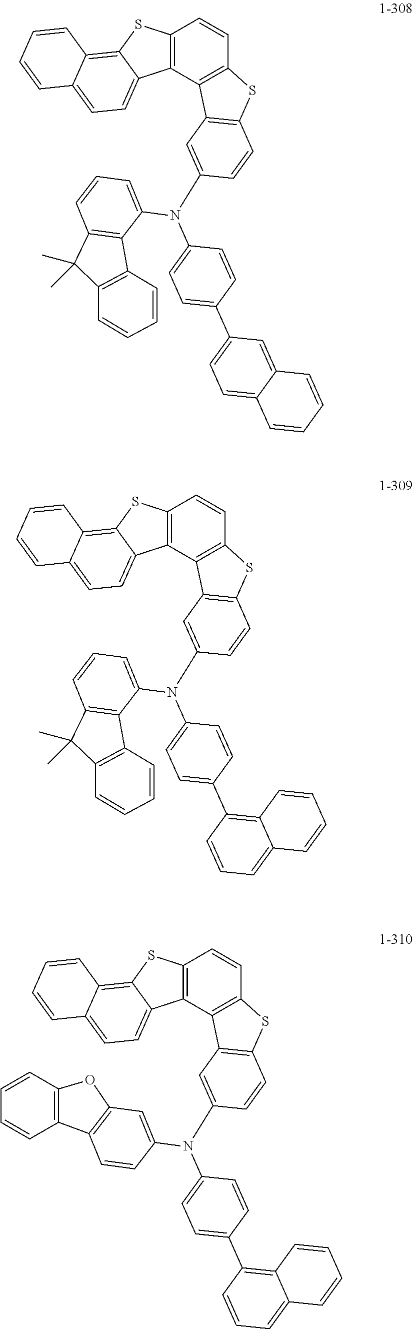 [SMILES: CC1(C)c2ccccc2-c2c(N(c3ccc(-c4ccc5ccccc5c4)cc3)c3ccc4sc5ccc6sc7c8ccccc8ccc7c6c5c4c3)cccc21 ; CC1(C)c2ccccc2-c2c(N(c3ccc(-c4cccc5ccccc45)cc3)c3ccc4sc5ccc6sc7c8ccccc8ccc7c6c5c4c3)cccc21 ; c1ccc2c(-c3ccc(N(c4ccc5c(c4)oc4ccccc45)c4ccc5sc6ccc7sc8c9ccccc9ccc8c7c6c5c4)cc3)cccc2c1]

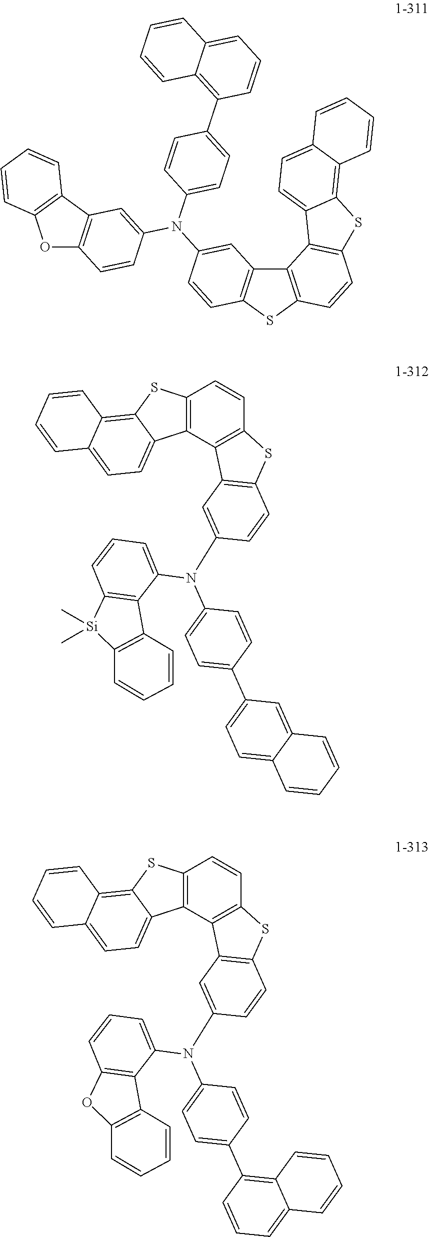 [SMILES: c1ccc2c(-c3ccc(N(c4ccc5oc6ccccc6c5c4)c4ccc5sc6ccc7sc8c9ccccc9ccc8c7c6c5c4)cc3)cccc2c1 ; C[Si]1(C)c2ccccc2-c2c(N(c3ccc(-c4ccc5ccccc5c4)cc3)c3ccc4sc5ccc6sc7c8ccccc8ccc7c6c5c4c3)cccc21 ; c1ccc2c(-c3ccc(N(c4ccc5sc6ccc7sc8c9ccccc9ccc8c7c6c5c4)c4cccc5oc6ccccc6c45)cc3)cccc2c1]

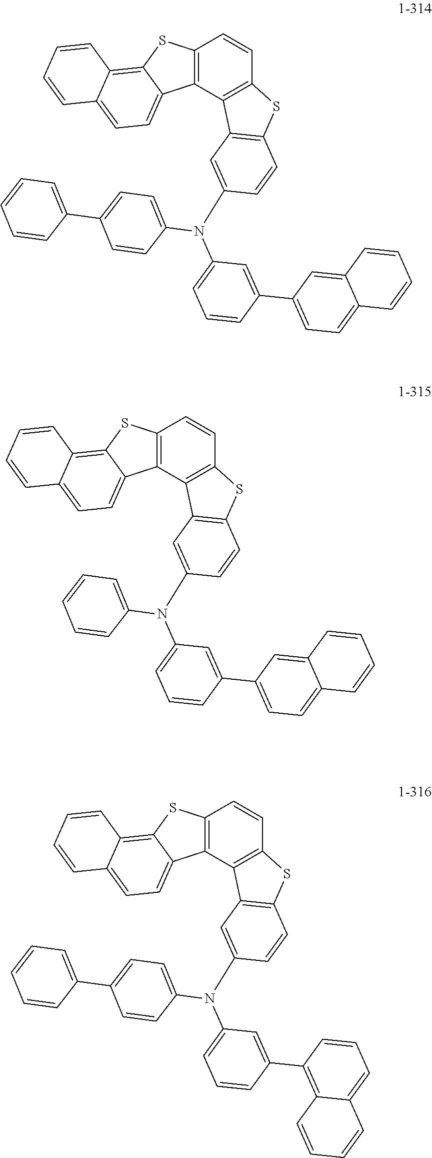 [SMILES: c1ccc(-c2ccc(N(c3cccc(-c4ccc5ccccc5c4)c3)c3ccc4sc5ccc6sc7c8ccccc8ccc7c6c5c4c3)cc2)cc1 ; c1ccc(N(c2cccc(-c3ccc4ccccc4c3)c2)c2ccc3sc4ccc5sc6c7ccccc7ccc6c5c4c3c2)cc1 ; c1ccc(-c2ccc(N(c3cccc(-c4cccc5ccccc45)c3)c3ccc4sc5ccc6sc7c8ccccc8ccc7c6c5c4c3)cc2)cc1]

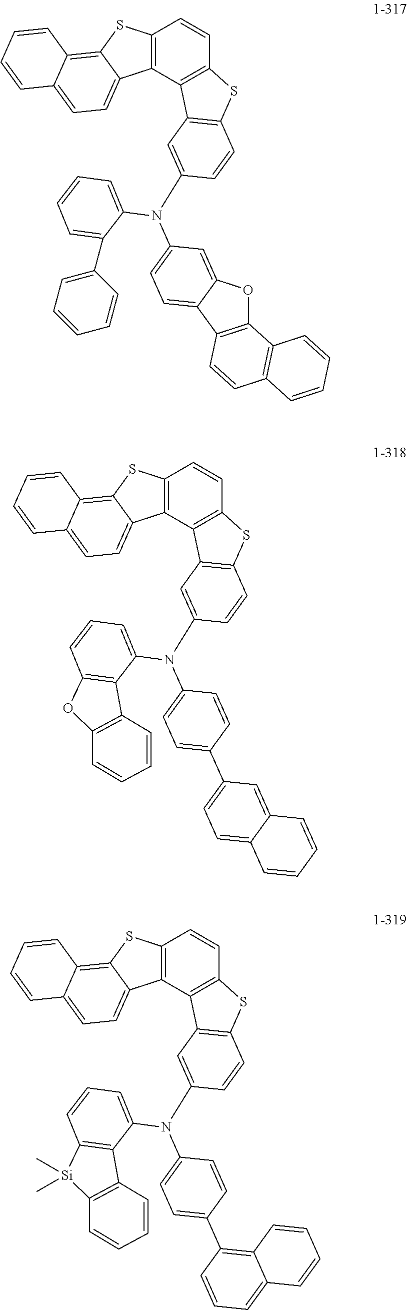 [SMILES: c1ccc(-c2ccccc2N(c2ccc3c(c2)oc2c4ccccc4ccc32)c2ccc3sc4ccc5sc6c7ccccc7ccc6c5c4c3c2)cc1 ; c1ccc2cc(-c3ccc(N(c4ccc5sc6ccc7sc8c9ccccc9ccc8c7c6c5c4)c4cccc5oc6ccccc6c45)cc3)ccc2c1 ; C[Si]1(C)c2ccccc2-c2c(N(c3ccc(-c4cccc5ccccc45)cc3)c3ccc4sc5ccc6sc7c8ccccc8ccc7c6c5c4c3)cccc21]

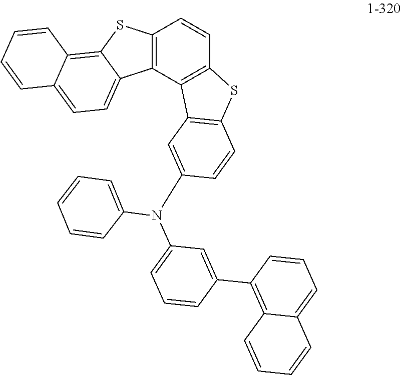 [SMILES: c1ccc(N(c2cccc(-c3cccc4ccccc34)c2)c2ccc3sc4ccc5sc6c7ccccc7ccc6c5c4c3c2)cc1]

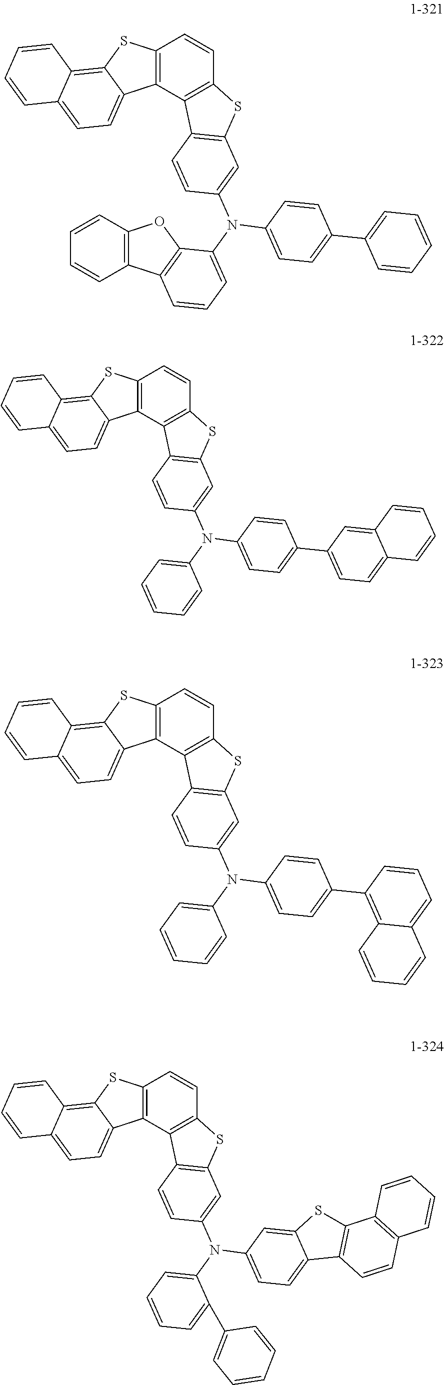 [SMILES: c1ccc(-c2ccc(N(c3ccc4c(c3)sc3ccc5sc6c7ccccc7ccc6c5c34)c3cccc4c3oc3ccccc34)cc2)cc1 ; c1ccc(N(c2ccc(-c3ccc4ccccc4c3)cc2)c2ccc3c(c2)sc2ccc4sc5c6ccccc6ccc5c4c23)cc1 ; c1ccc(N(c2ccc(-c3cccc4ccccc34)cc2)c2ccc3c(c2)sc2ccc4sc5c6ccccc6ccc5c4c23)cc1 ; c1ccc(-c2ccccc2N(c2ccc3c(c2)sc2c4ccccc4ccc32)c2ccc3c(c2)sc2ccc4sc5c6ccccc6ccc5c4c23)cc1]

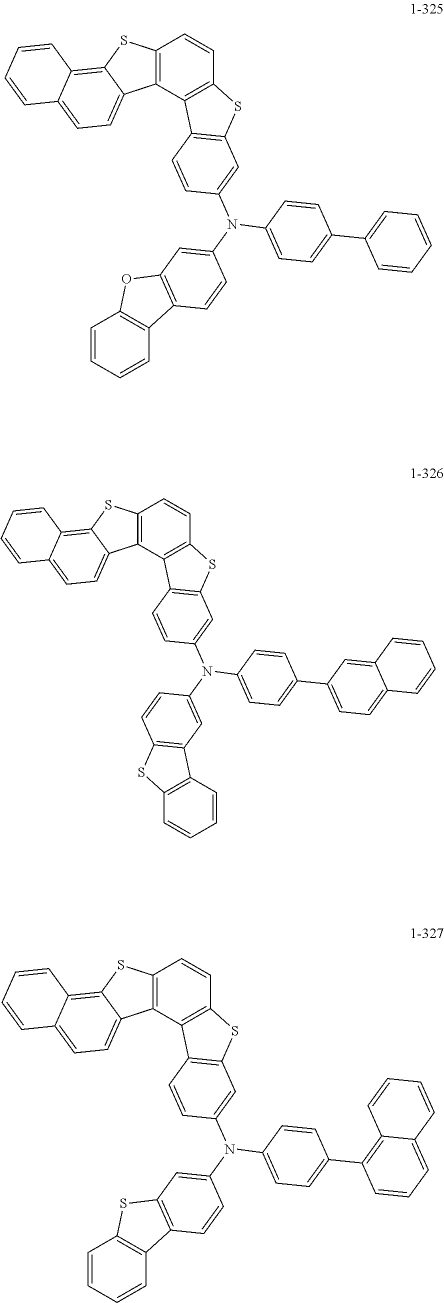 [SMILES: c1ccc(-c2ccc(N(c3ccc4c(c3)oc3ccccc34)c3ccc4c(c3)sc3ccc5sc6c7ccccc7ccc6c5c34)cc2)cc1 ; c1ccc2cc(-c3ccc(N(c4ccc5c(c4)sc4ccc6sc7c8ccccc8ccc7c6c45)c4ccc5sc6ccccc6c5c4)cc3)ccc2c1 ; c1ccc2c(-c3ccc(N(c4ccc5c(c4)sc4ccccc45)c4ccc5c(c4)sc4ccc6sc7c8ccccc8ccc7c6c45)cc3)cccc2c1]

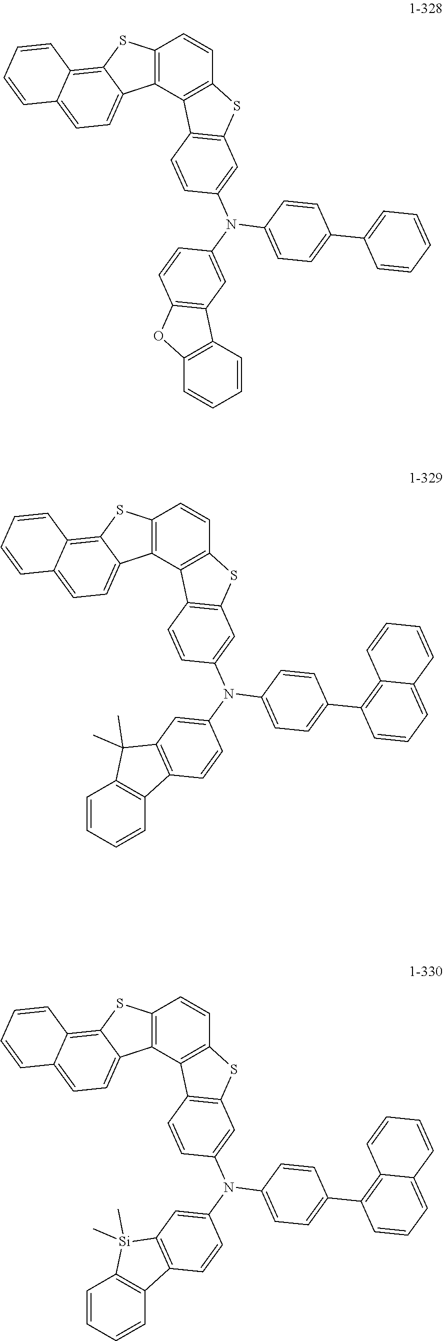 [SMILES: c1ccc(-c2ccc(N(c3ccc4c(c3)sc3ccc5sc6c7ccccc7ccc6c5c34)c3ccc4oc5ccccc5c4c3)cc2)cc1 ; CC1(C)c2ccccc2-c2ccc(N(c3ccc(-c4cccc5ccccc45)cc3)c3ccc4c(c3)sc3ccc5sc6c7ccccc7ccc6c5c34)cc21 ; C[Si]1(C)c2ccccc2-c2ccc(N(c3ccc(-c4cccc5ccccc45)cc3)c3ccc4c(c3)sc3ccc5sc6c7ccccc7ccc6c5c34)cc21]

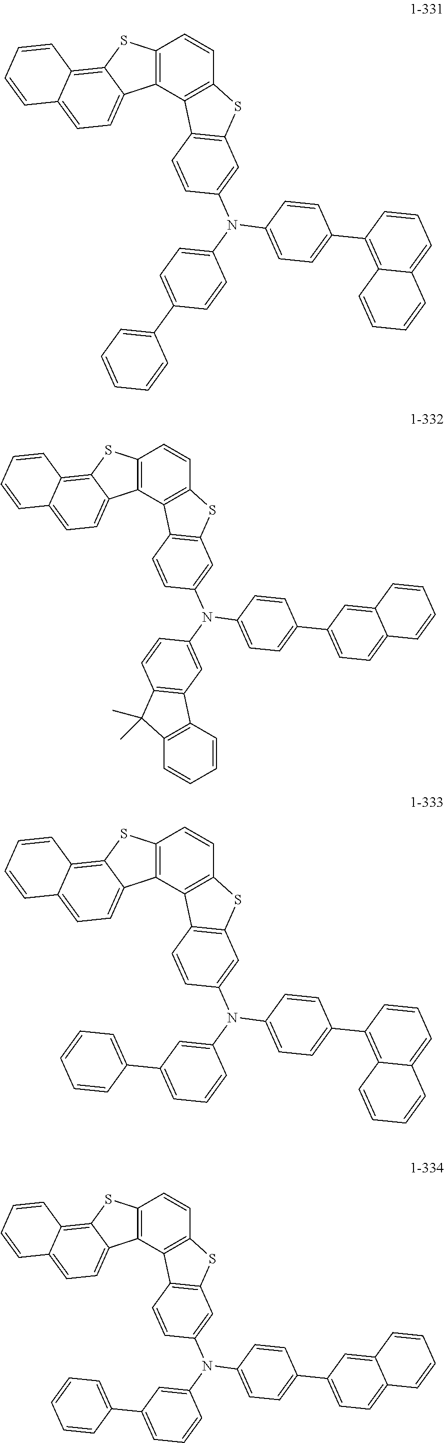 [SMILES: c1ccc(-c2ccc(N(c3ccc(-c4cccc5ccccc45)cc3)c3ccc4c(c3)sc3ccc5sc6c7ccccc7ccc6c5c34)cc2)cc1 ; CC1(C)c2ccccc2-c2cc(N(c3ccc(-c4ccc5ccccc5c4)cc3)c3ccc4c(c3)sc3ccc5sc6c7ccccc7ccc6c5c34)ccc21 ; c1ccc(-c2cccc(N(c3ccc(-c4cccc5ccccc45)cc3)c3ccc4c(c3)sc3ccc5sc6c7ccccc7ccc6c5c34)c2)cc1 ; c1ccc(-c2cccc(N(c3ccc(-c4ccc5ccccc5c4)cc3)c3ccc4c(c3)sc3ccc5sc6c7ccccc7ccc6c5c34)c2)cc1]

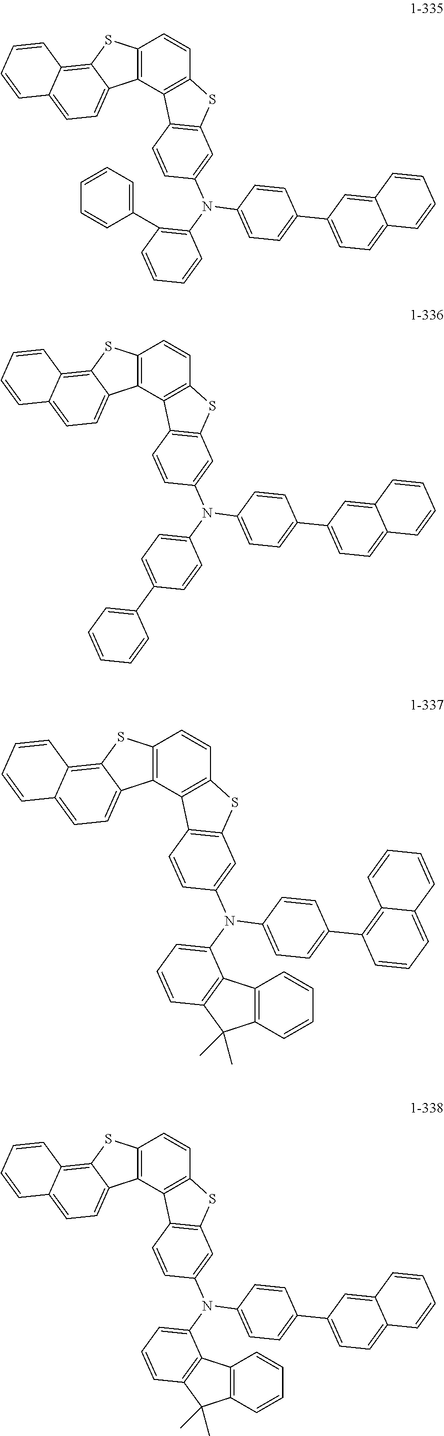 [SMILES: c1ccc(-c2ccccc2N(c2ccc(-c3ccc4ccccc4c3)cc2)c2ccc3c(c2)sc2ccc4sc5c6ccccc6ccc5c4c23)cc1 ; c1ccc(-c2ccc(N(c3ccc(-c4ccc5ccccc5c4)cc3)c3ccc4c(c3)sc3ccc5sc6c7ccccc7ccc6c5c34)cc2)cc1 ; CC1(C)c2ccccc2-c2c(N(c3ccc(-c4cccc5ccccc45)cc3)c3ccc4c(c3)sc3ccc5sc6c7ccccc7ccc6c5c34)cccc21 ; CC1(C)c2ccccc2-c2c(N(c3ccc(-c4ccc5ccccc5c4)cc3)c3ccc4c(c3)sc3ccc5sc6c7ccccc7ccc6c5c34)cccc21]

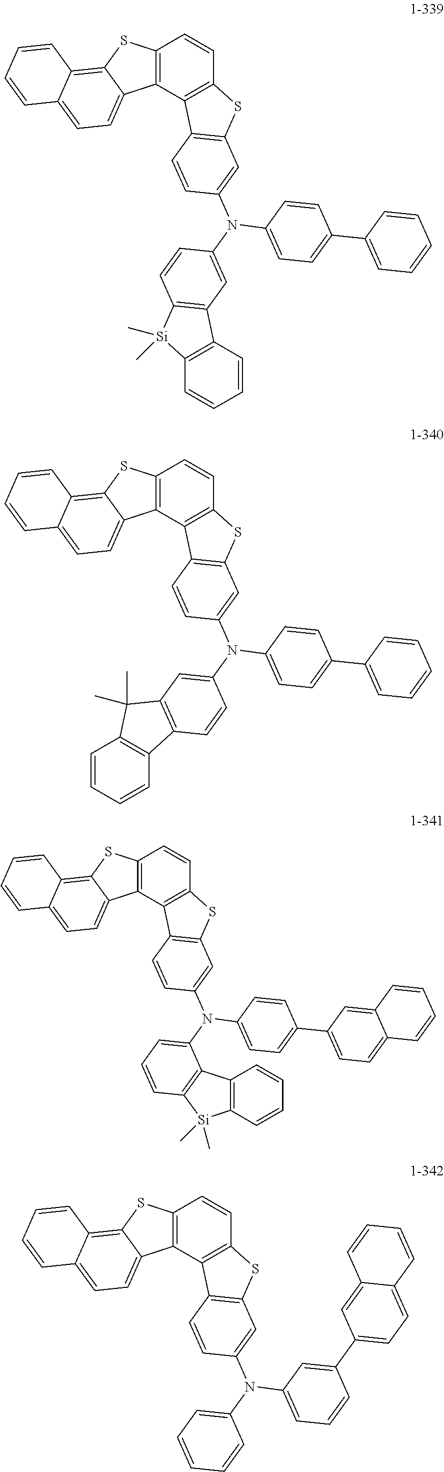 [SMILES: C[Si]1(C)c2ccccc2-c2cc(N(c3ccc(-c4ccccc4)cc3)c3ccc4c(c3)sc3ccc5sc6c7ccccc7ccc6c5c34)ccc21 ; CC1(C)c2ccccc2-c2ccc(N(c3ccc(-c4ccccc4)cc3)c3ccc4c(c3)sc3ccc5sc6c7ccccc7ccc6c5c34)cc21 ; C[Si]1(C)c2ccccc2-c2c(N(c3ccc(-c4ccc5ccccc5c4)cc3)c3ccc4c(c3)sc3ccc5sc6c7ccccc7ccc6c5c34)cccc21 ; c1ccc(N(c2cccc(-c3ccc4ccccc4c3)c2)c2ccc3c(c2)sc2ccc4sc5c6ccccc6ccc5c4c23)cc1]

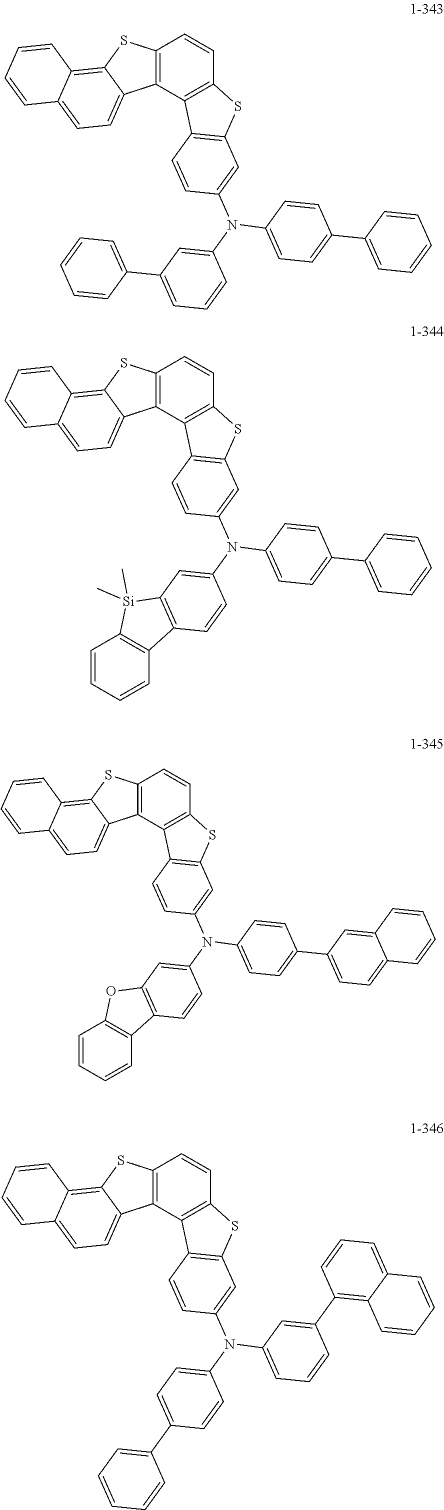 [SMILES: c1ccc(-c2ccc(N(c3cccc(-c4ccccc4)c3)c3ccc4c(c3)sc3ccc5sc6c7ccccc7ccc6c5c34)cc2)cc1 ; C[Si]1(C)c2ccccc2-c2ccc(N(c3ccc(-c4ccccc4)cc3)c3ccc4c(c3)sc3ccc5sc6c7ccccc7ccc6c5c34)cc21 ; c1ccc2cc(-c3ccc(N(c4ccc5c(c4)oc4ccccc45)c4ccc5c(c4)sc4ccc6sc7c8ccccc8ccc7c6c45)cc3)ccc2c1 ; c1ccc(-c2ccc(N(c3cccc(-c4cccc5ccccc45)c3)c3ccc4c(c3)sc3ccc5sc6c7ccccc7ccc6c5c34)cc2)cc1]

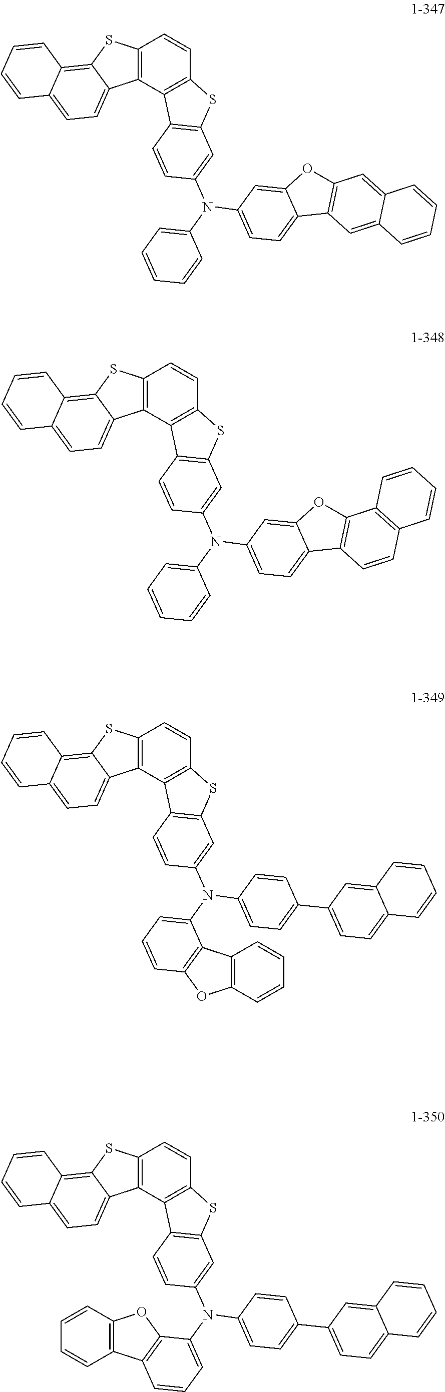 [SMILES: c1ccc(N(c2ccc3c(c2)oc2cc4ccccc4cc23)c2ccc3c(c2)sc2ccc4sc5c6ccccc6ccc5c4c23)cc1 ; c1ccc(N(c2ccc3c(c2)oc2c4ccccc4ccc32)c2ccc3c(c2)sc2ccc4sc5c6ccccc6ccc5c4c23)cc1 ; c1ccc2cc(-c3ccc(N(c4ccc5c(c4)sc4ccc6sc7c8ccccc8ccc7c6c45)c4cccc5oc6ccccc6c45)cc3)ccc2c1 ; c1ccc2cc(-c3ccc(N(c4ccc5c(c4)sc4ccc6sc7c8ccccc8ccc7c6c45)c4cccc5c4oc4ccccc45)cc3)ccc2c1]

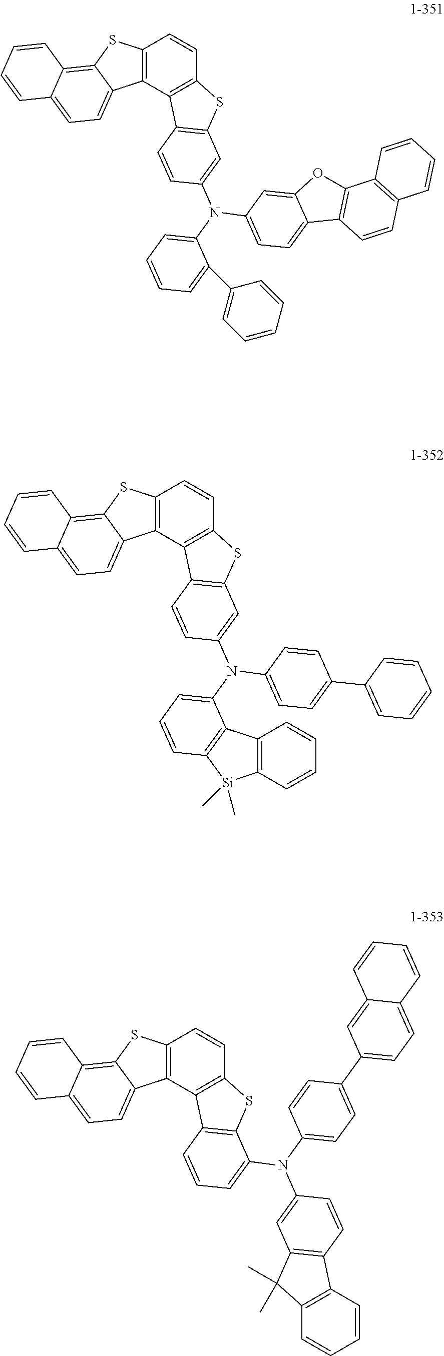 [SMILES: c1ccc(-c2ccccc2N(c2ccc3c(c2)oc2c4ccccc4ccc32)c2ccc3c(c2)sc2ccc4sc5c6ccccc6ccc5c4c23)cc1 ; C[Si]1(C)c2ccccc2-c2c(N(c3ccc(-c4ccccc4)cc3)c3ccc4c(c3)sc3ccc5sc6c7ccccc7ccc6c5c34)cccc21 ; CC1(C)c2ccccc2-c2ccc(N(c3ccc(-c4ccc5ccccc5c4)cc3)c3cccc4c3sc3ccc5sc6c7ccccc7ccc6c5c34)cc21]

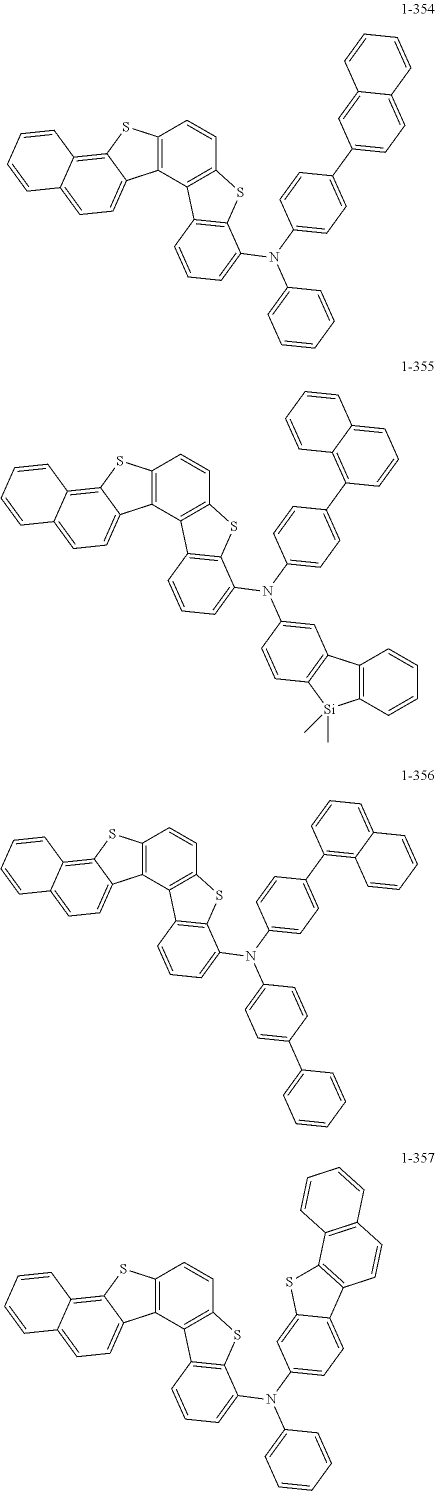

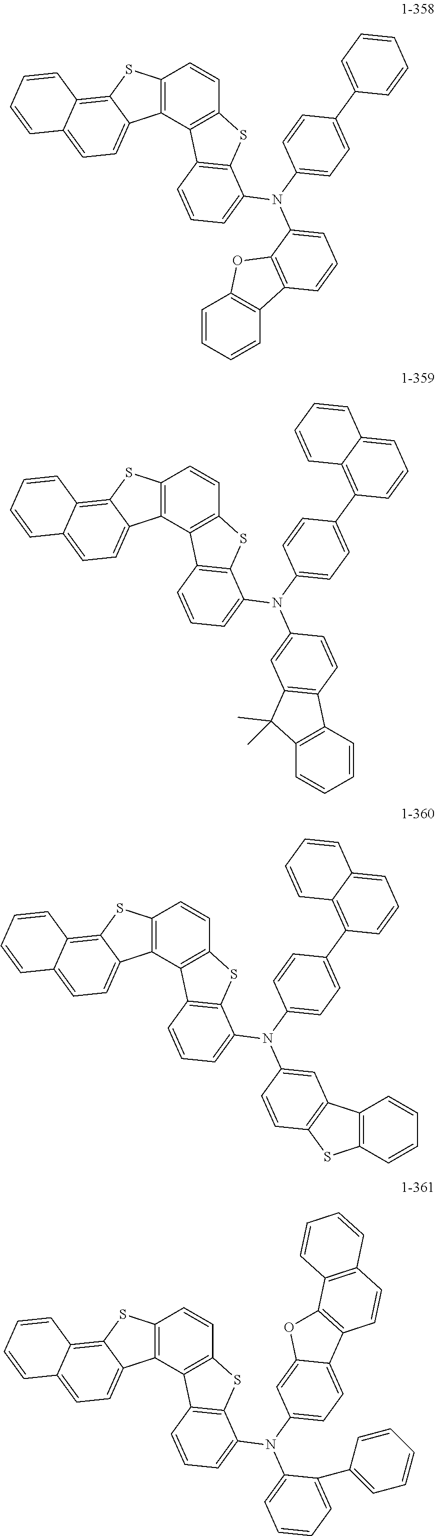 [SMILES: c1ccc(-c2ccc(N(c3cccc4c3oc3ccccc34)c3cccc4c3sc3ccc5sc6c7ccccc7ccc6c5c34)cc2)cc1 ; CC1(C)c2ccccc2-c2ccc(N(c3ccc(-c4cccc5ccccc45)cc3)c3cccc4c3sc3ccc5sc6c7ccccc7ccc6c5c34)cc21 ; c1ccc2c(-c3ccc(N(c4ccc5sc6ccccc6c5c4)c4cccc5c4sc4ccc6sc7c8ccccc8ccc7c6c45)cc3)cccc2c1 ; c1ccc(-c2ccccc2N(c2ccc3c(c2)oc2c4ccccc4ccc32)c2cccc3c2sc2ccc4sc5c6ccccc6ccc5c4c23)cc1]

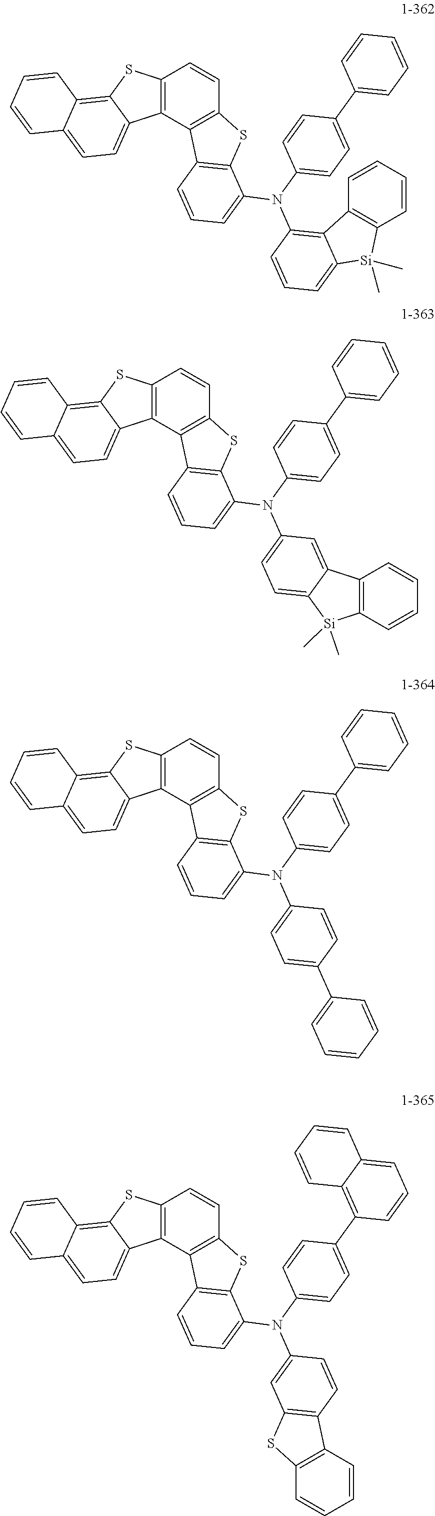 [SMILES: C[Si]1(C)c2ccccc2-c2c(N(c3ccc(-c4ccccc4)cc3)c3cccc4c3sc3ccc5sc6c7ccccc7ccc6c5c34)cccc21 ; C[Si]1(C)c2ccccc2-c2cc(N(c3ccc(-c4ccccc4)cc3)c3cccc4c3sc3ccc5sc6c7ccccc7ccc6c5c34)ccc21 ; c1ccc(-c2ccc(N(c3ccc(-c4ccccc4)cc3)c3cccc4c3sc3ccc5sc6c7ccccc7ccc6c5c34)cc2)cc1 ; c1ccc2c(-c3ccc(N(c4ccc5c(c4)sc4ccccc45)c4cccc5c4sc4ccc6sc7c8ccccc8ccc7c6c45)cc3)cccc2c1]

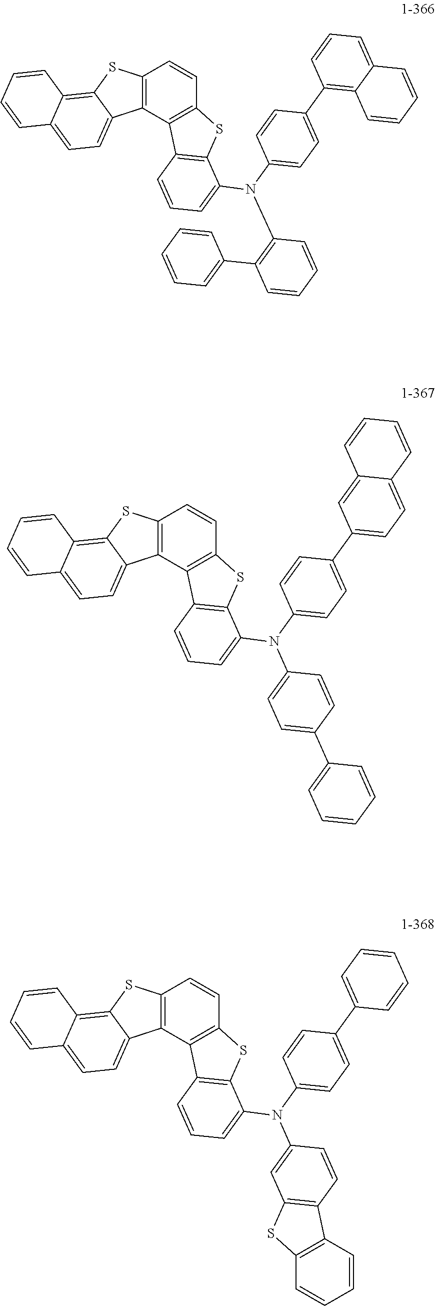 [SMILES: c1ccc(-c2ccccc2N(c2ccc(-c3cccc4ccccc34)cc2)c2cccc3c2sc2ccc4sc5c6ccccc6ccc5c4c23)cc1 ; c1ccc(-c2ccc(N(c3ccc(-c4ccc5ccccc5c4)cc3)c3cccc4c3sc3ccc5sc6c7ccccc7ccc6c5c34)cc2)cc1 ; c1ccc(-c2ccc(N(c3ccc4c(c3)sc3ccccc34)c3cccc4c3sc3ccc5sc6c7ccccc7ccc6c5c34)cc2)cc1]

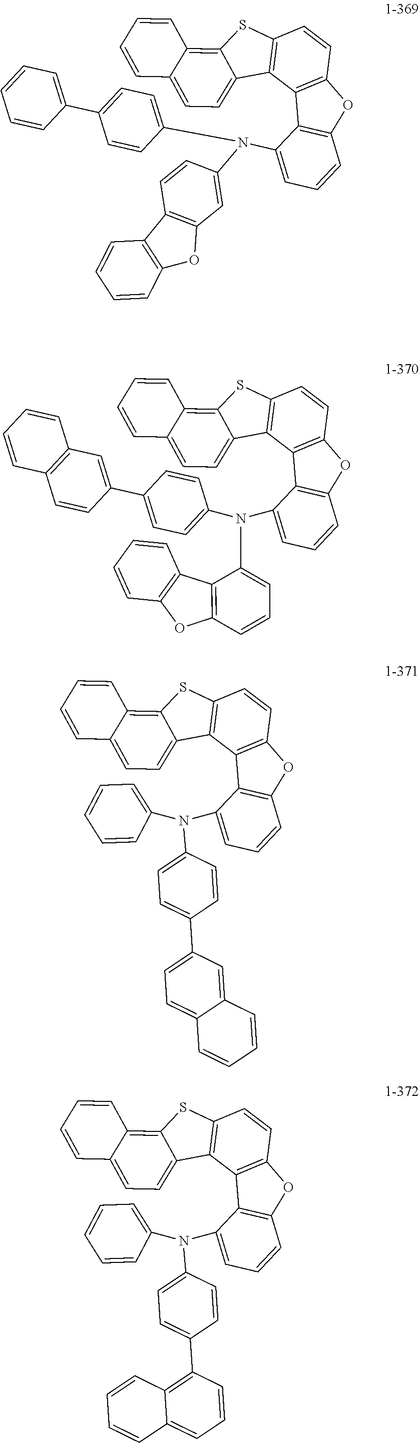

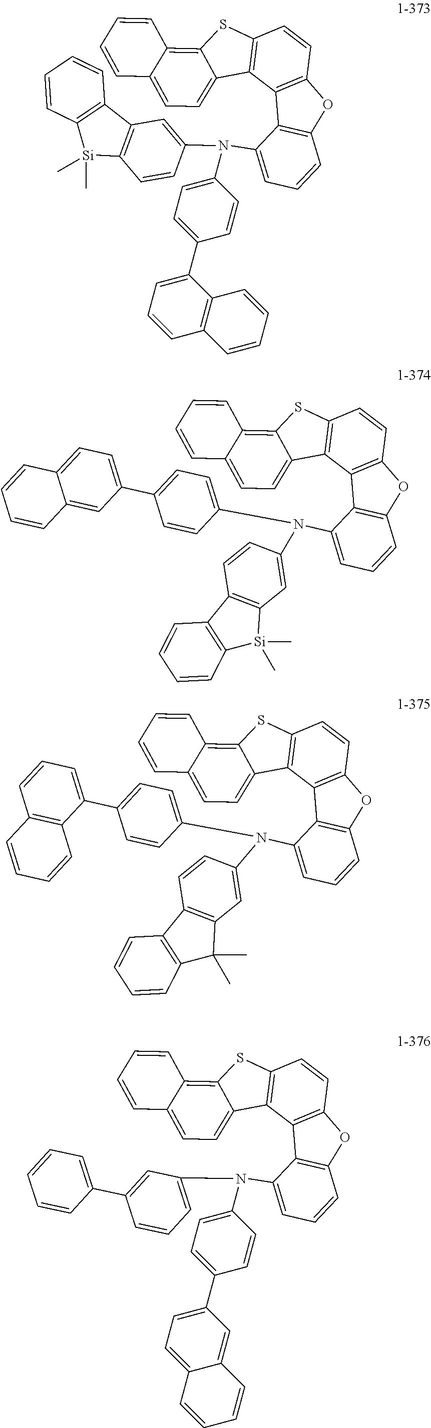

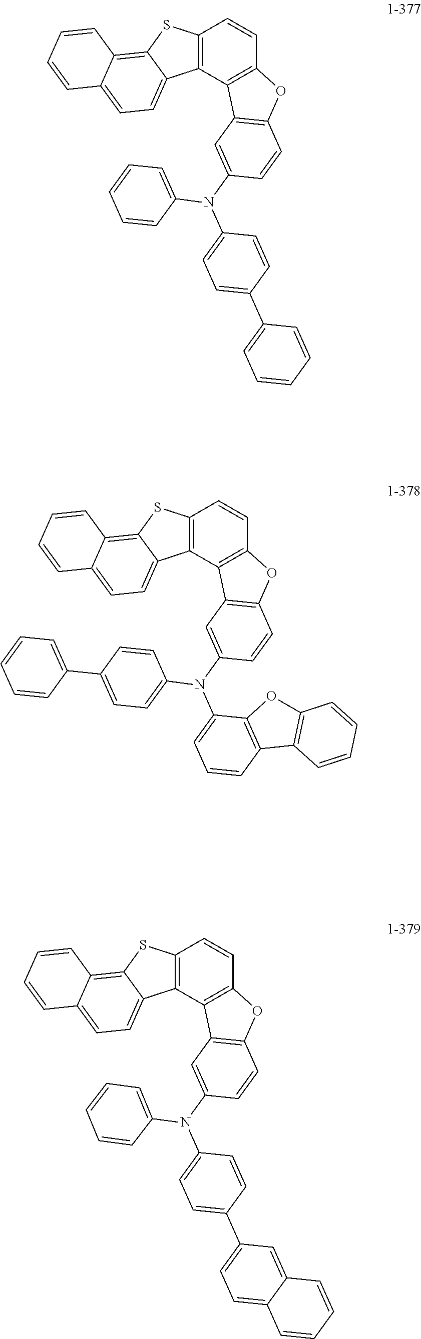 [SMILES: c1ccc(-c2ccc(N(c3ccccc3)c3ccc4oc5ccc6sc7c8ccccc8ccc7c6c5c4c3)cc2)cc1 ; c1ccc(-c2ccc(N(c3ccc4oc5ccc6sc7c8ccccc8ccc7c6c5c4c3)c3cccc4c3oc3ccccc34)cc2)cc1 ; c1ccc(N(c2ccc(-c3ccc4ccccc4c3)cc2)c2ccc3oc4ccc5sc6c7ccccc7ccc6c5c4c3c2)cc1]

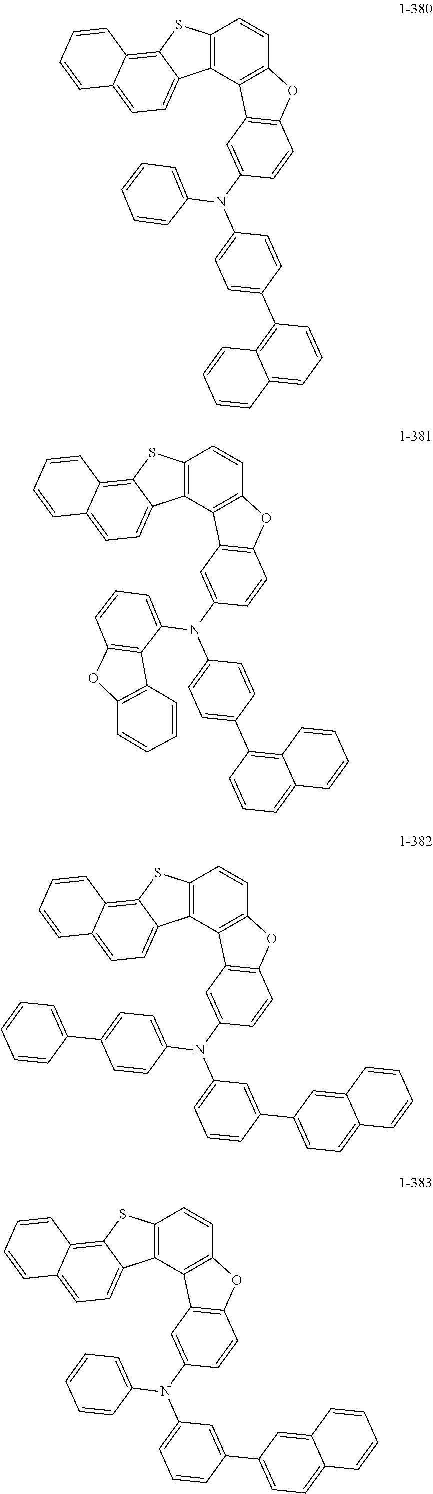

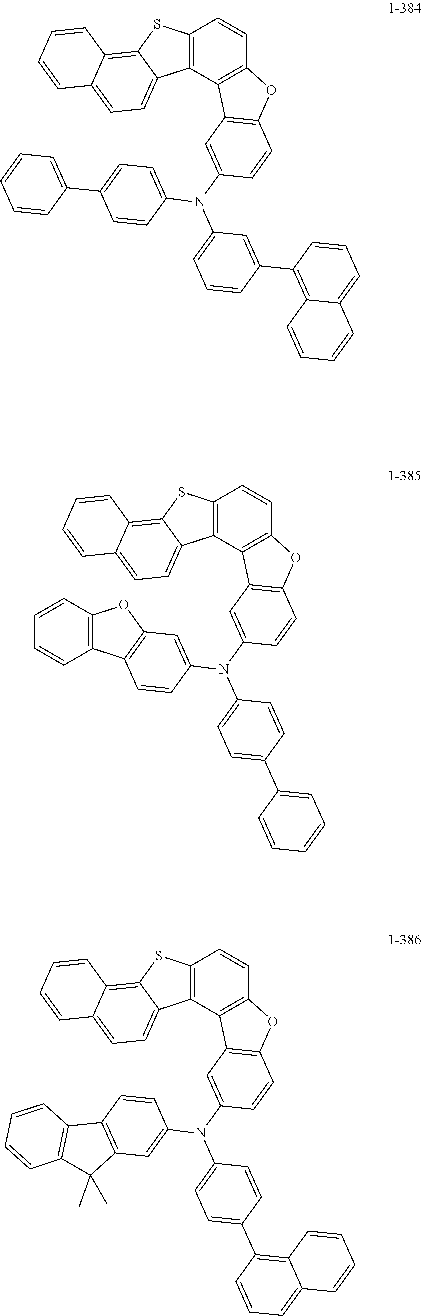 [SMILES: c1ccc(-c2ccc(N(c3cccc(-c4cccc5ccccc45)c3)c3ccc4oc5ccc6sc7c8ccccc8ccc7c6c5c4c3)cc2)cc1 ; c1ccc(-c2ccc(N(c3ccc4c(c3)oc3ccccc34)c3ccc4oc5ccc6sc7c8ccccc8ccc7c6c5c4c3)cc2)cc1 ; CC1(C)c2ccccc2-c2ccc(N(c3ccc(-c4cccc5ccccc45)cc3)c3ccc4oc5ccc6sc7c8ccccc8ccc7c6c5c4c3)cc21]

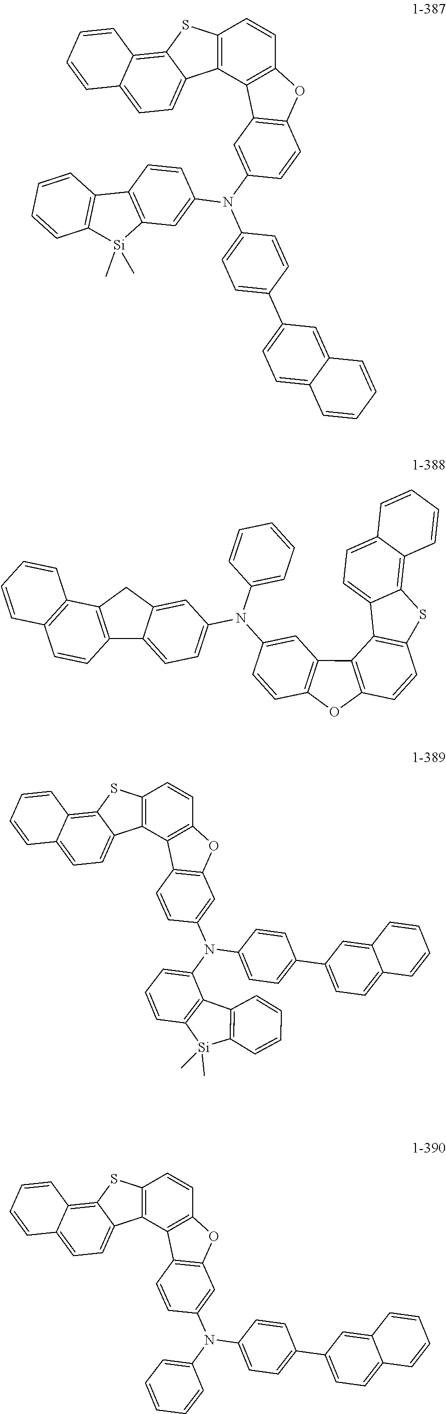 [SMILES: C[Si]1(C)c2ccccc2-c2ccc(N(c3ccc(-c4ccc5ccccc5c4)cc3)c3ccc4oc5ccc6sc7c8ccccc8ccc7c6c5c4c3)cc21 ; c1ccc(N(c2ccc3c(c2)Cc2c-3ccc3ccccc23)c2ccc3oc4ccc5sc6c7ccccc7ccc6c5c4c3c2)cc1 ; C[Si]1(C)c2ccccc2-c2c(N(c3ccc(-c4ccc5ccccc5c4)cc3)c3ccc4c(c3)oc3ccc5sc6c7ccccc7ccc6c5c34)cccc21 ; c1ccc(N(c2ccc(-c3ccc4ccccc4c3)cc2)c2ccc3c(c2)oc2ccc4sc5c6ccccc6ccc5c4c23)cc1]

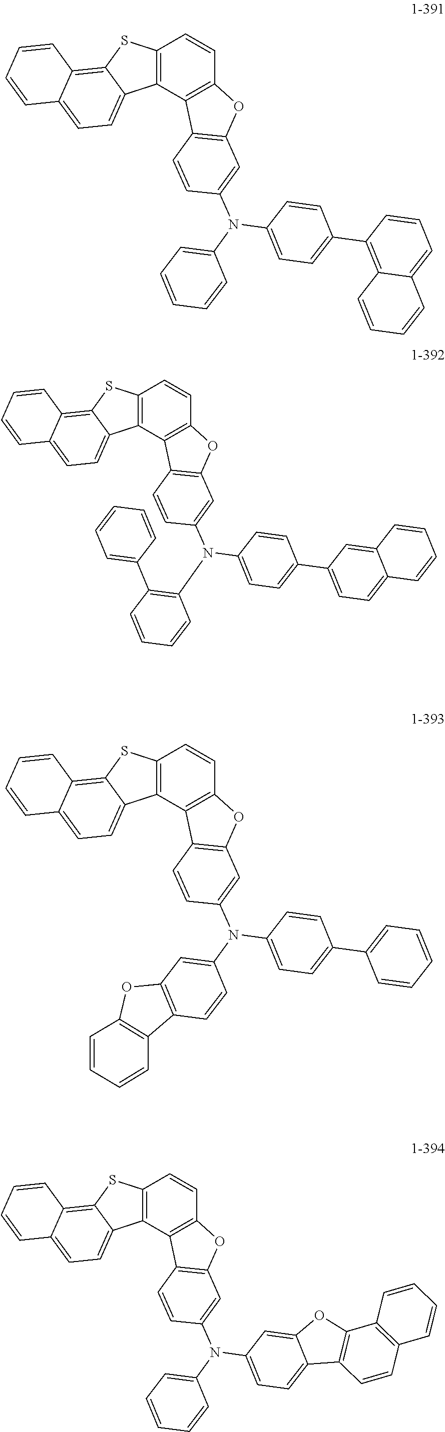 [SMILES: c1ccc(N(c2ccc(-c3cccc4ccccc34)cc2)c2ccc3c(c2)oc2ccc4sc5c6ccccc6ccc5c4c23)cc1 ; c1ccc(-c2ccccc2N(c2ccc(-c3ccc4ccccc4c3)cc2)c2ccc3c(c2)oc2ccc4sc5c6ccccc6ccc5c4c23)cc1 ; c1ccc(-c2ccc(N(c3ccc4c(c3)oc3ccccc34)c3ccc4c(c3)oc3ccc5sc6c7ccccc7ccc6c5c34)cc2)cc1 ; c1ccc(N(c2ccc3c(c2)oc2c4ccccc4ccc32)c2ccc3c(c2)oc2ccc4sc5c6ccccc6ccc5c4c23)cc1]

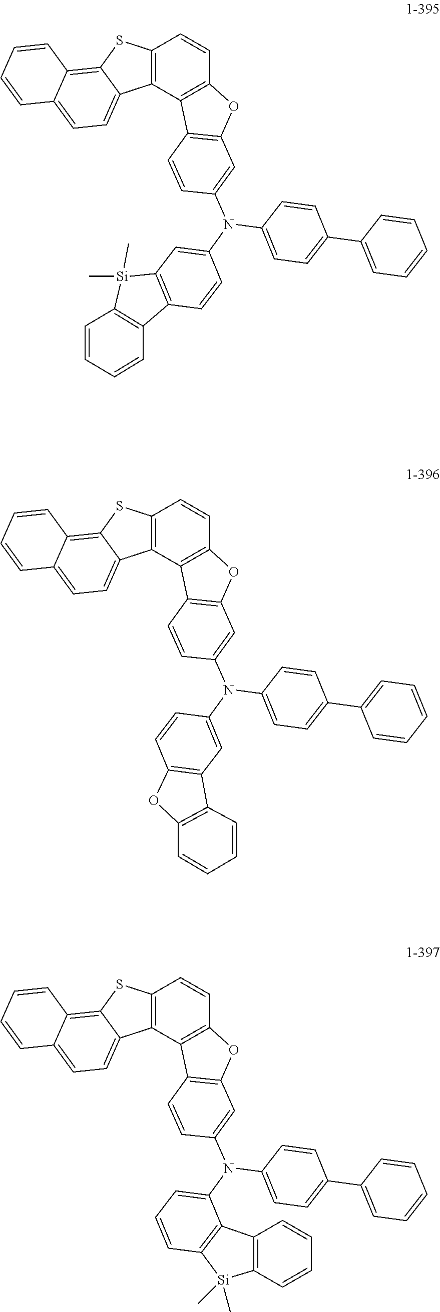 [SMILES: C[Si]1(C)c2ccccc2-c2ccc(N(c3ccc(-c4ccccc4)cc3)c3ccc4c(c3)oc3ccc5sc6c7ccccc7ccc6c5c34)cc21 ; c1ccc(-c2ccc(N(c3ccc4c(c3)oc3ccc5sc6c7ccccc7ccc6c5c34)c3ccc4oc5ccccc5c4c3)cc2)cc1 ; C[Si]1(C)c2ccccc2-c2c(N(c3ccc(-c4ccccc4)cc3)c3ccc4c(c3)oc3ccc5sc6c7ccccc7ccc6c5c34)cccc21]

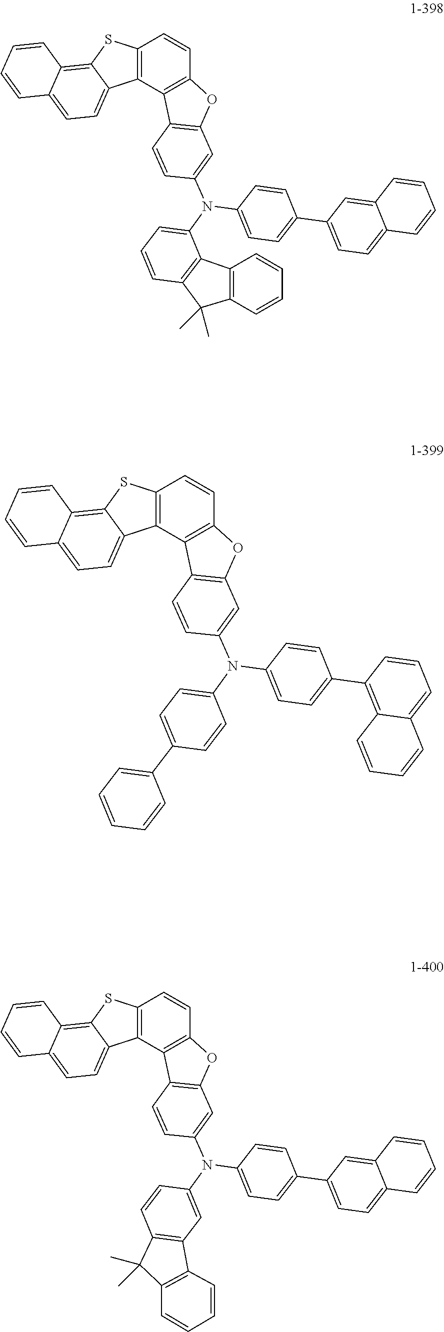 [SMILES: CC1(C)c2ccccc2-c2c(N(c3ccc(-c4ccc5ccccc5c4)cc3)c3ccc4c(c3)oc3ccc5sc6c7ccccc7ccc6c5c34)cccc21 ; c1ccc(-c2ccc(N(c3ccc(-c4cccc5ccccc45)cc3)c3ccc4c(c3)oc3ccc5sc6c7ccccc7ccc6c5c34)cc2)cc1 ; CC1(C)c2ccccc2-c2cc(N(c3ccc(-c4ccc5ccccc5c4)cc3)c3ccc4c(c3)oc3ccc5sc6c7ccccc7ccc6c5c34)ccc21]

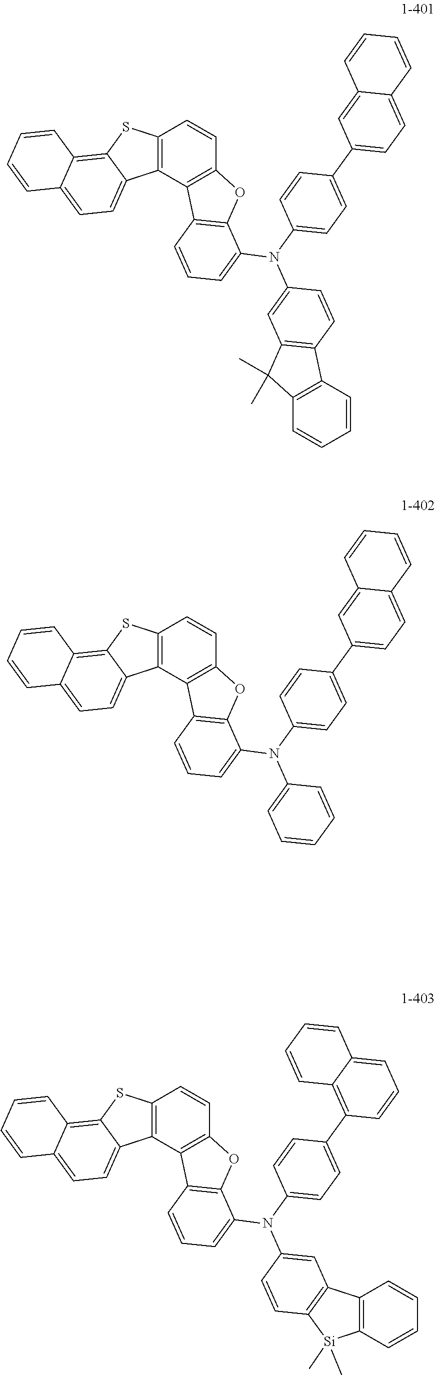 [SMILES: CC1(C)c2ccccc2-c2ccc(N(c3ccc(-c4ccc5ccccc5c4)cc3)c3cccc4c3oc3ccc5sc6c7ccccc7ccc6c5c34)cc21 ; c1ccc(N(c2ccc(-c3ccc4ccccc4c3)cc2)c2cccc3c2oc2ccc4sc5c6ccccc6ccc5c4c23)cc1 ; C[Si]1(C)c2ccccc2-c2cc(N(c3ccc(-c4cccc5ccccc45)cc3)c3cccc4c3oc3ccc5sc6c7ccccc7ccc6c5c34)ccc21]

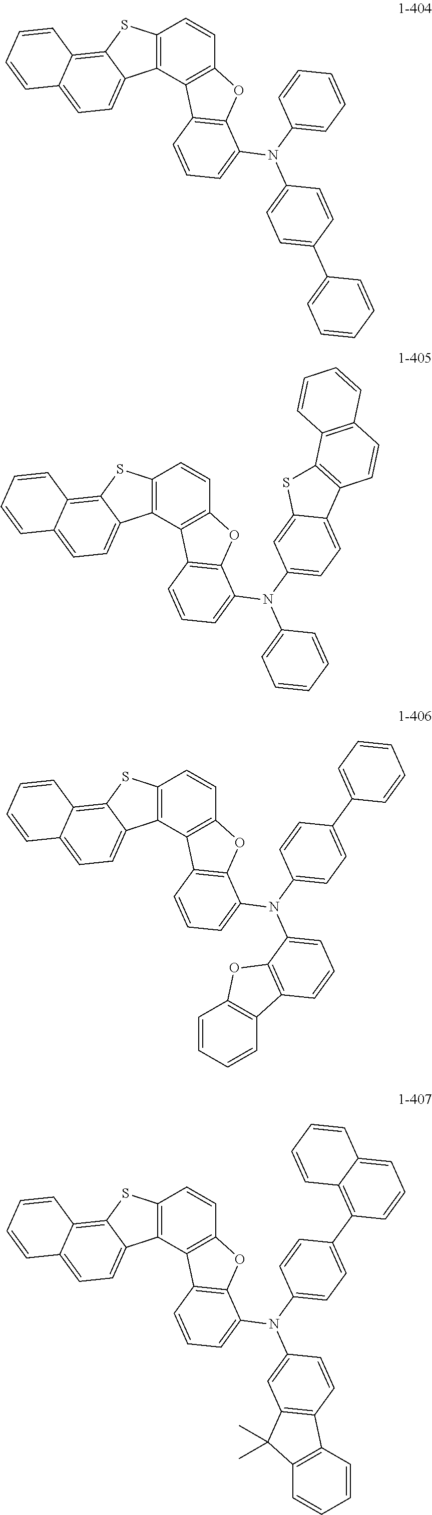

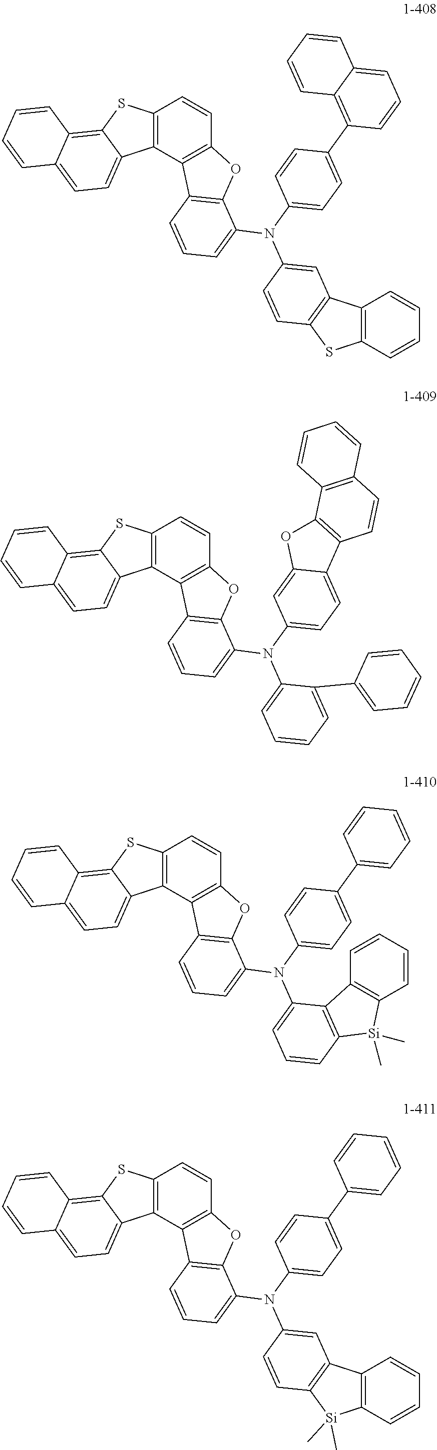 [SMILES: c1ccc2c(-c3ccc(N(c4ccc5sc6ccccc6c5c4)c4cccc5c4oc4ccc6sc7c8ccccc8ccc7c6c45)cc3)cccc2c1 ; c1ccc(-c2ccccc2N(c2ccc3c(c2)oc2c4ccccc4ccc32)c2cccc3c2oc2ccc4sc5c6ccccc6ccc5c4c23)cc1 ; C[Si]1(C)c2ccccc2-c2c(N(c3ccc(-c4ccccc4)cc3)c3cccc4c3oc3ccc5sc6c7ccccc7ccc6c5c34)cccc21 ; C[Si]1(C)c2ccccc2-c2cc(N(c3ccc(-c4ccccc4)cc3)c3cccc4c3oc3ccc5sc6c7ccccc7ccc6c5c34)ccc21]

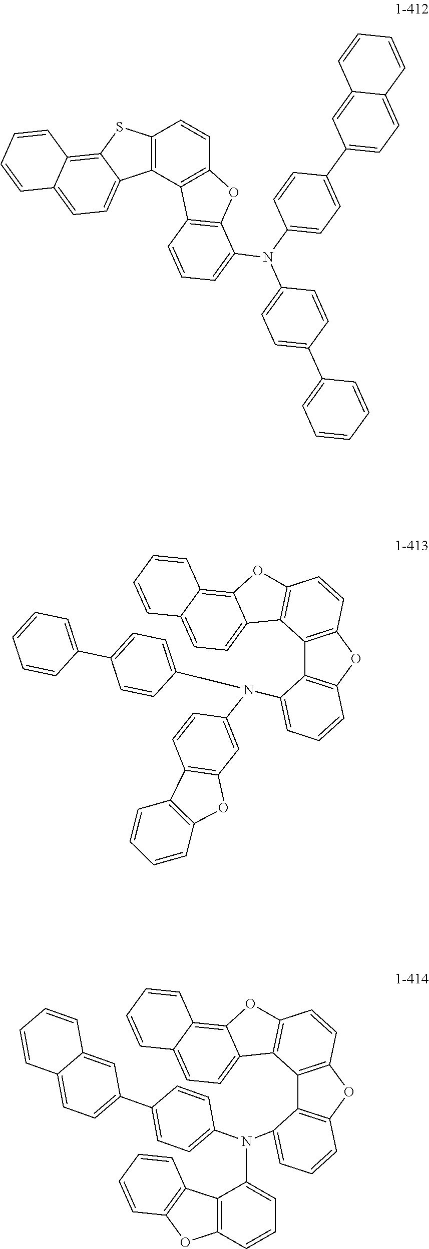 [SMILES: c1ccc(-c2ccc(N(c3ccc(-c4ccc5ccccc5c4)cc3)c3cccc4c3oc3ccc5sc6c7ccccc7ccc6c5c34)cc2)cc1 ; c1ccc(-c2ccc(N(c3ccc4c(c3)oc3ccccc34)c3cccc4oc5ccc6oc7c8ccccc8ccc7c6c5c34)cc2)cc1 ; c1ccc2cc(-c3ccc(N(c4cccc5oc6ccccc6c45)c4cccc5oc6ccc7oc8c9ccccc9ccc8c7c6c45)cc3)ccc2c1]

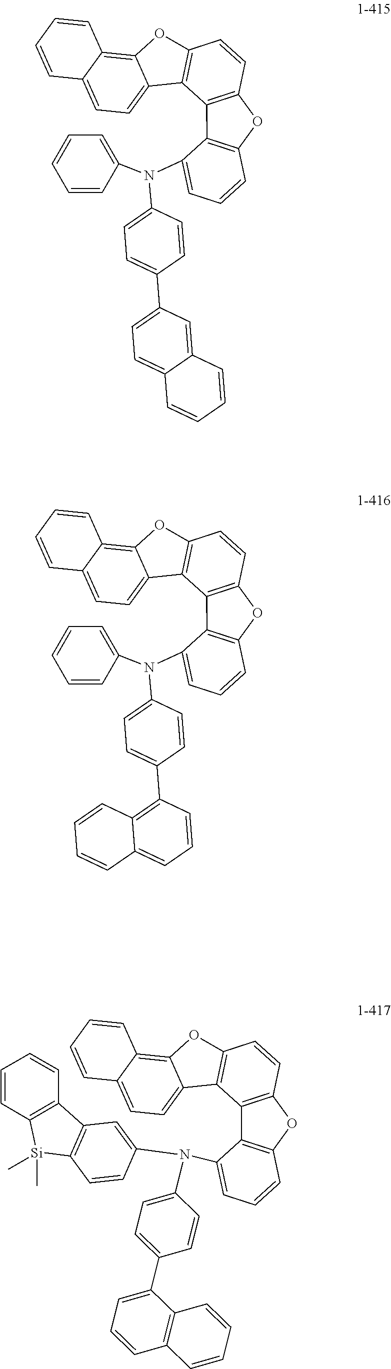 [SMILES: c1ccc(N(c2ccc(-c3ccc4ccccc4c3)cc2)c2cccc3oc4ccc5oc6c7ccccc7ccc6c5c4c23)cc1 ; c1ccc(N(c2ccc(-c3cccc4ccccc34)cc2)c2cccc3oc4ccc5oc6c7ccccc7ccc6c5c4c23)cc1 ; C[Si]1(C)c2ccccc2-c2cc(N(c3ccc(-c4cccc5ccccc45)cc3)c3cccc4oc5ccc6oc7c8ccccc8ccc7c6c5c34)ccc21]

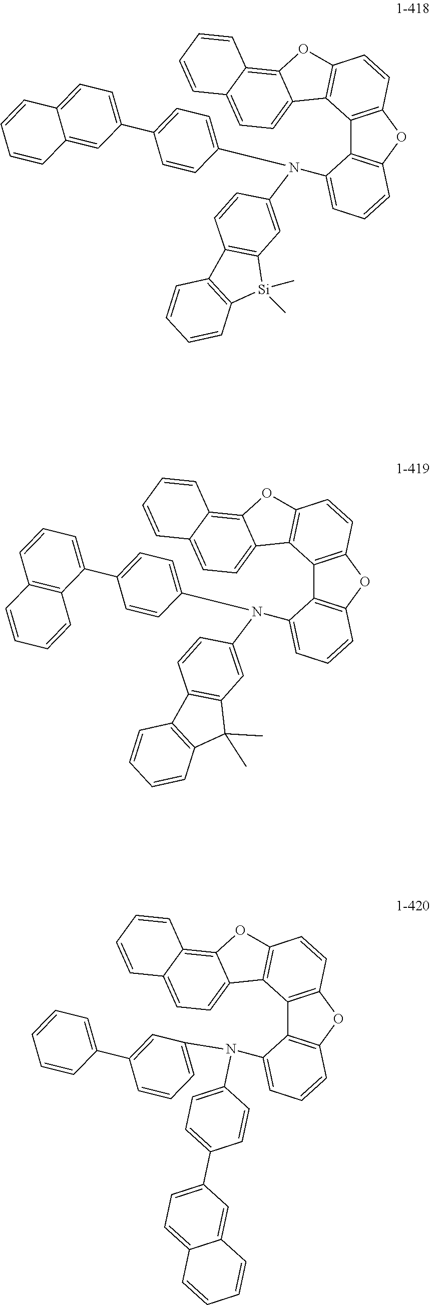 [SMILES: C[Si]1(C)c2ccccc2-c2ccc(N(c3ccc(-c4ccc5ccccc5c4)cc3)c3cccc4oc5ccc6oc7c8ccccc8ccc7c6c5c34)cc21 ; CC1(C)c2ccccc2-c2ccc(N(c3ccc(-c4cccc5ccccc45)cc3)c3cccc4oc5ccc6oc7c8ccccc8ccc7c6c5c34)cc21 ; c1ccc(-c2cccc(N(c3ccc(-c4ccc5ccccc5c4)cc3)c3cccc4oc5ccc6oc7c8ccccc8ccc7c6c5c34)c2)cc1]

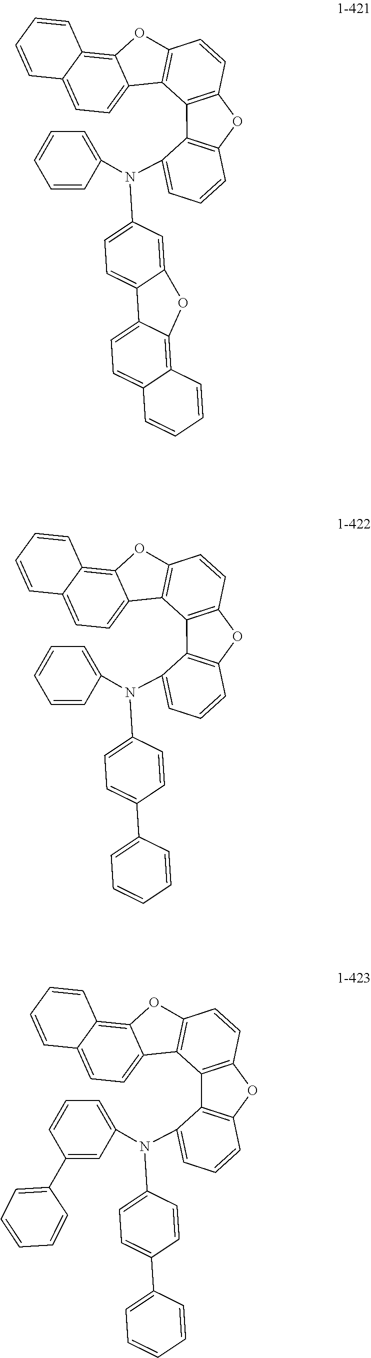 [SMILES: c1ccc(N(c2ccc3c(c2)oc2c4ccccc4ccc32)c2cccc3oc4ccc5oc6c7ccccc7ccc6c5c4c23)cc1 ; c1ccc(-c2ccc(N(c3ccccc3)c3cccc4oc5ccc6oc7c8ccccc8ccc7c6c5c34)cc2)cc1 ; c1ccc(-c2ccc(N(c3cccc(-c4ccccc4)c3)c3cccc4oc5ccc6oc7c8ccccc8ccc7c6c5c34)cc2)cc1]

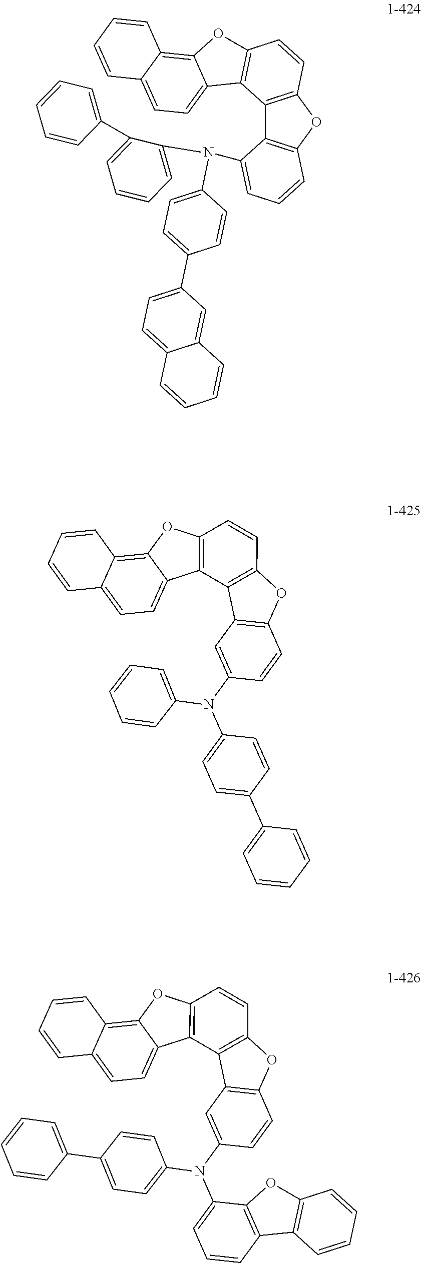 [SMILES: c1ccc(-c2ccccc2N(c2ccc(-c3ccc4ccccc4c3)cc2)c2cccc3oc4ccc5oc6c7ccccc7ccc6c5c4c23)cc1 ; c1ccc(-c2ccc(N(c3ccccc3)c3ccc4oc5ccc6oc7c8ccccc8ccc7c6c5c4c3)cc2)cc1 ; c1ccc(-c2ccc(N(c3ccc4oc5ccc6oc7c8ccccc8ccc7c6c5c4c3)c3cccc4c3oc3ccccc34)cc2)cc1]

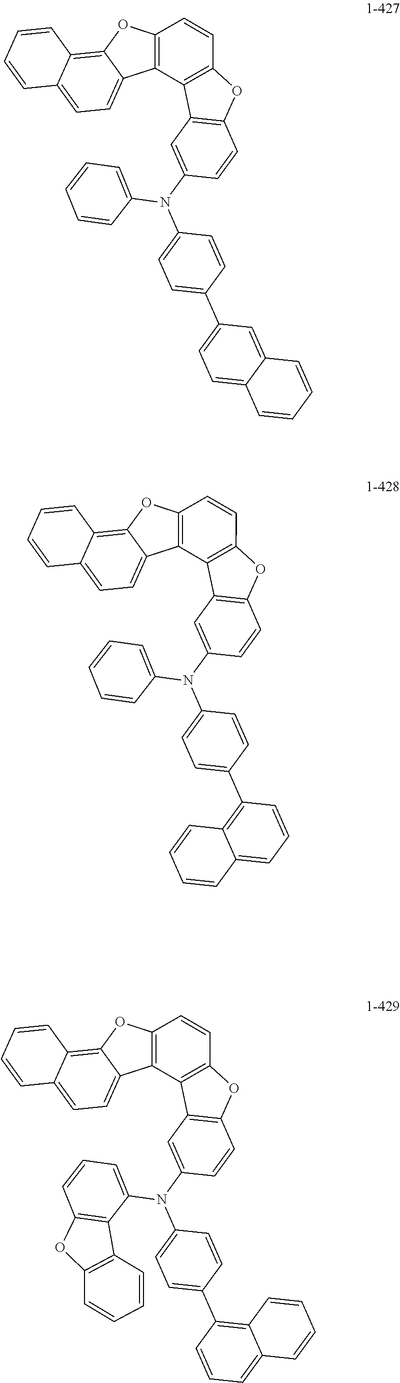 [SMILES: c1ccc(N(c2ccc(-c3ccc4ccccc4c3)cc2)c2ccc3oc4ccc5oc6c7ccccc7ccc6c5c4c3c2)cc1 ; c1ccc(N(c2ccc(-c3cccc4ccccc34)cc2)c2ccc3oc4ccc5oc6c7ccccc7ccc6c5c4c3c2)cc1 ; c1ccc2c(-c3ccc(N(c4ccc5oc6ccc7oc8c9ccccc9ccc8c7c6c5c4)c4cccc5oc6ccccc6c45)cc3)cccc2c1]

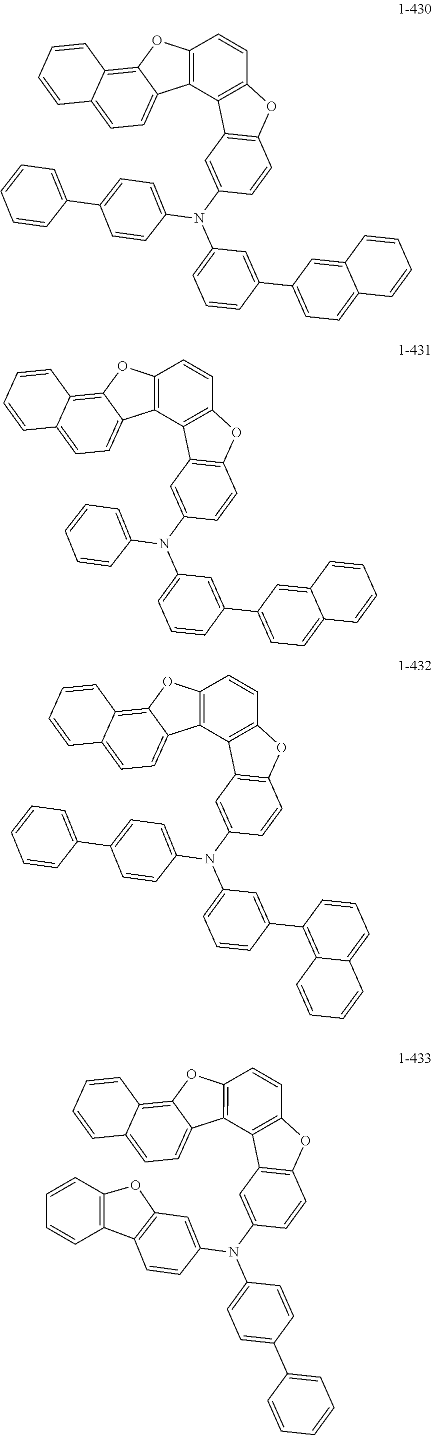 [SMILES: c1ccc(-c2ccc(N(c3cccc(-c4ccc5ccccc5c4)c3)c3ccc4oc5ccc6oc7c8ccccc8ccc7c6c5c4c3)cc2)cc1 ; c1ccc(N(c2cccc(-c3ccc4ccccc4c3)c2)c2ccc3oc4ccc5oc6c7ccccc7ccc6c5c4c3c2)cc1 ; c1ccc(-c2ccc(N(c3cccc(-c4cccc5ccccc45)c3)c3ccc4oc5ccc6oc7c8ccccc8ccc7c6c5c4c3)cc2)cc1 ; c1ccc(-c2ccc(N(c3ccc4c(c3)oc3ccccc34)c3ccc4oc5ccc6oc7c8ccccc8ccc7c6c5c4c3)cc2)cc1]

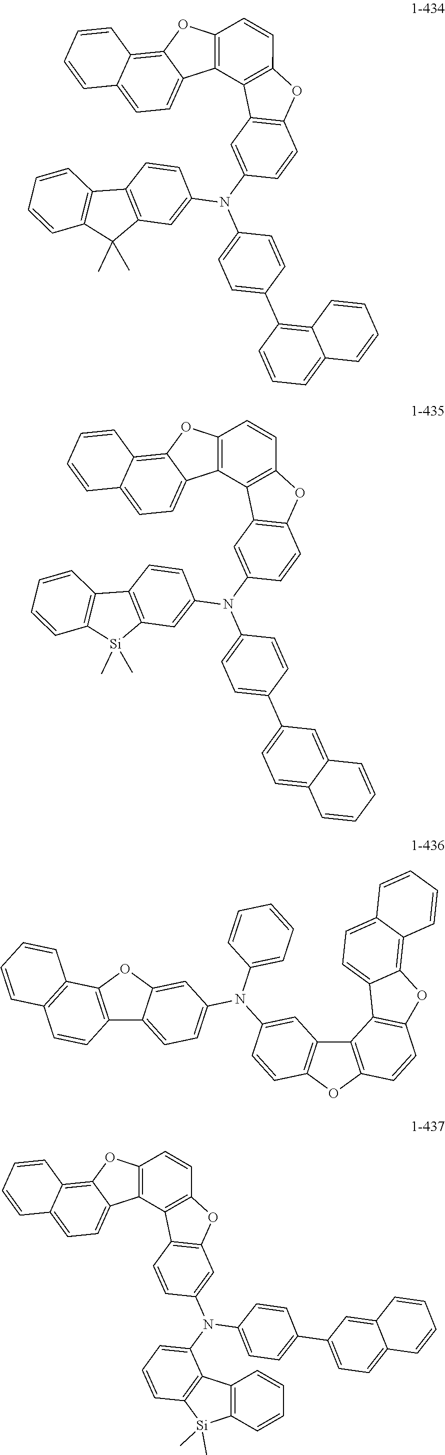 [SMILES: CC1(C)c2ccccc2-c2ccc(N(c3ccc(-c4cccc5ccccc45)cc3)c3ccc4oc5ccc6oc7c8ccccc8ccc7c6c5c4c3)cc21 ; C[Si]1(C)c2ccccc2-c2ccc(N(c3ccc(-c4ccc5ccccc5c4)cc3)c3ccc4oc5ccc6oc7c8ccccc8ccc7c6c5c4c3)cc21 ; c1ccc(N(c2ccc3c(c2)oc2c4ccccc4ccc32)c2ccc3oc4ccc5oc6c7ccccc7ccc6c5c4c3c2)cc1 ; C[Si]1(C)c2ccccc2-c2c(N(c3ccc(-c4ccc5ccccc5c4)cc3)c3ccc4c(c3)oc3ccc5oc6c7ccccc7ccc6c5c34)cccc21]

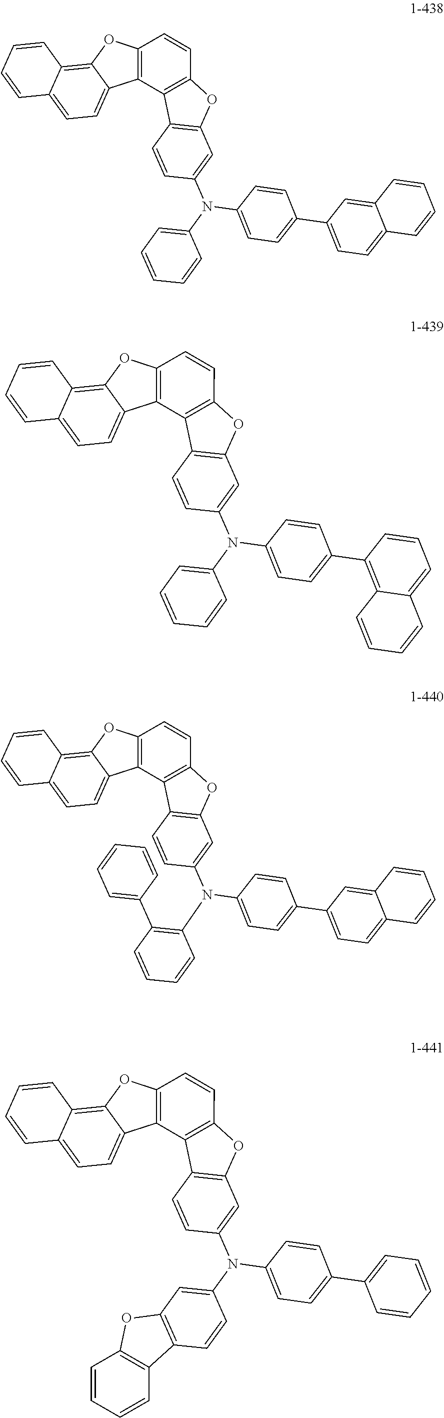 [SMILES: c1ccc(N(c2ccc(-c3ccc4ccccc4c3)cc2)c2ccc3c(c2)oc2ccc4oc5c6ccccc6ccc5c4c23)cc1 ; c1ccc(N(c2ccc(-c3cccc4ccccc34)cc2)c2ccc3c(c2)oc2ccc4oc5c6ccccc6ccc5c4c23)cc1 ; c1ccc(-c2ccccc2N(c2ccc(-c3ccc4ccccc4c3)cc2)c2ccc3c(c2)oc2ccc4oc5c6ccccc6ccc5c4c23)cc1 ; c1ccc(-c2ccc(N(c3ccc4c(c3)oc3ccccc34)c3ccc4c(c3)oc3ccc5oc6c7ccccc7ccc6c5c34)cc2)cc1]

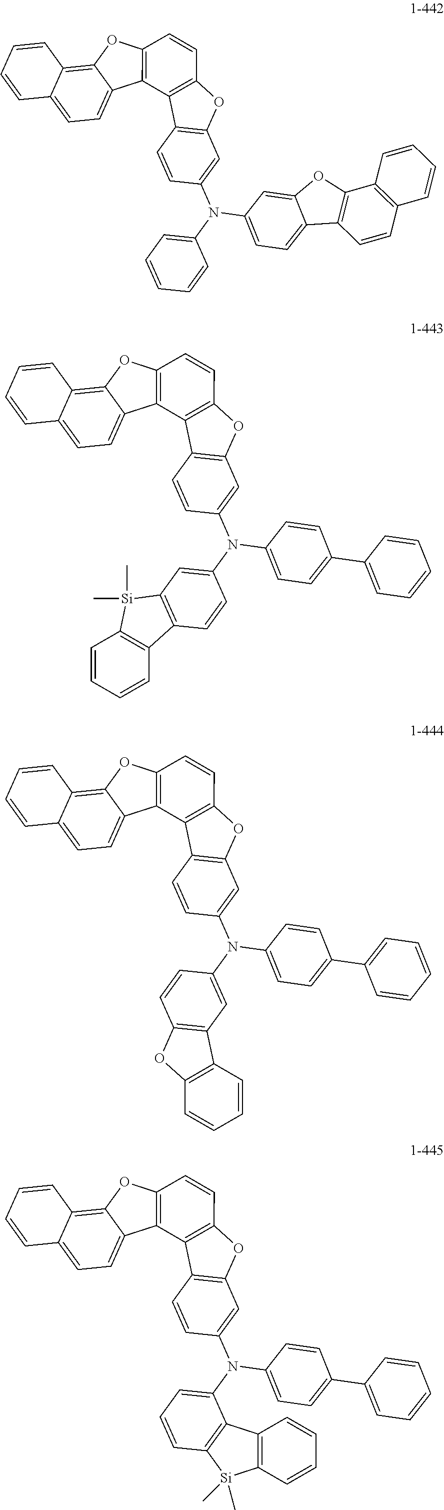 [SMILES: c1ccc(N(c2ccc3c(c2)oc2c4ccccc4ccc32)c2ccc3c(c2)oc2ccc4oc5c6ccccc6ccc5c4c23)cc1 ; C[Si]1(C)c2ccccc2-c2ccc(N(c3ccc(-c4ccccc4)cc3)c3ccc4c(c3)oc3ccc5oc6c7ccccc7ccc6c5c34)cc21 ; c1ccc(-c2ccc(N(c3ccc4c(c3)oc3ccc5oc6c7ccccc7ccc6c5c34)c3ccc4oc5ccccc5c4c3)cc2)cc1 ; C[Si]1(C)c2ccccc2-c2c(N(c3ccc(-c4ccccc4)cc3)c3ccc4c(c3)oc3ccc5oc6c7ccccc7ccc6c5c34)cccc21]

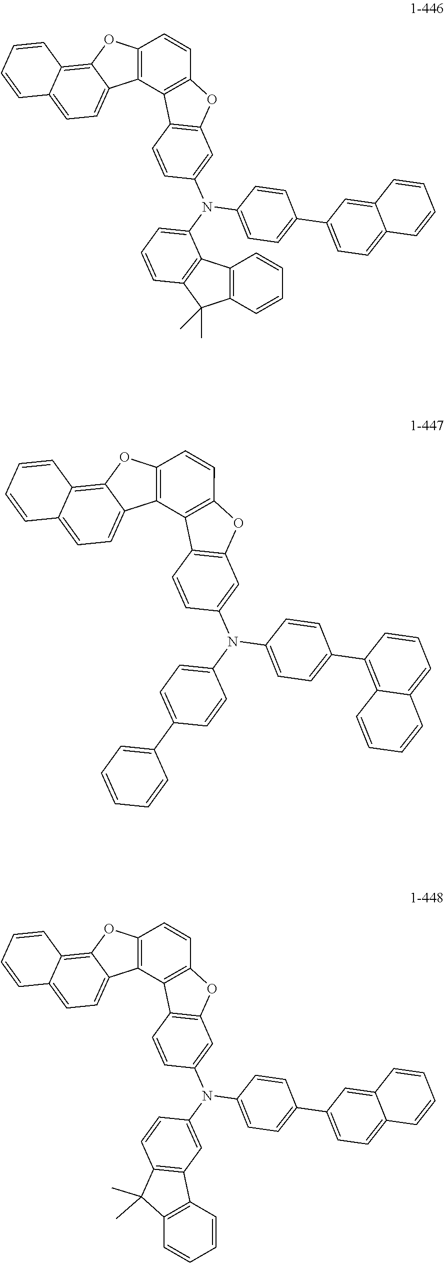 [SMILES: CC1(C)c2ccccc2-c2c(N(c3ccc(-c4ccc5ccccc5c4)cc3)c3ccc4c(c3)oc3ccc5oc6c7ccccc7ccc6c5c34)cccc21 ; c1ccc(-c2ccc(N(c3ccc(-c4cccc5ccccc45)cc3)c3ccc4c(c3)oc3ccc5oc6c7ccccc7ccc6c5c34)cc2)cc1 ; CC1(C)c2ccccc2-c2cc(N(c3ccc(-c4ccc5ccccc5c4)cc3)c3ccc4c(c3)oc3ccc5oc6c7ccccc7ccc6c5c34)ccc21]

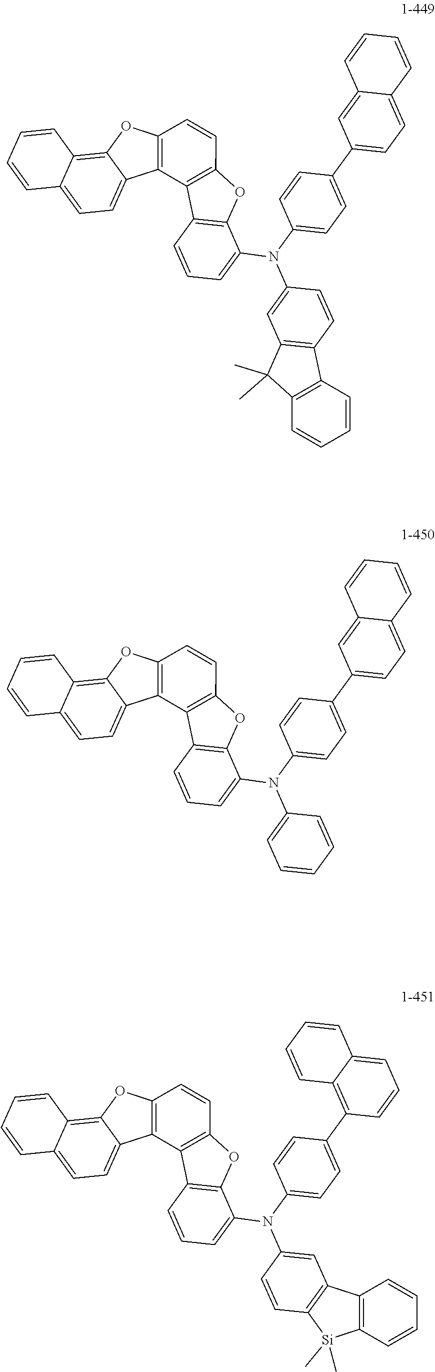 [SMILES: CC1(C)c2ccccc2-c2ccc(N(c3ccc(-c4ccc5ccccc5c4)cc3)c3cccc4c3oc3ccc5oc6c7ccccc7ccc6c5c34)cc21 ; c1ccc(N(c2ccc(-c3ccc4ccccc4c3)cc2)c2cccc3c2oc2ccc4oc5c6ccccc6ccc5c4c23)cc1 ; C[Si]1(C)c2ccccc2-c2cc(N(c3ccc(-c4cccc5ccccc45)cc3)c3cccc4c3oc3ccc5oc6c7ccccc7ccc6c5c34)ccc21]

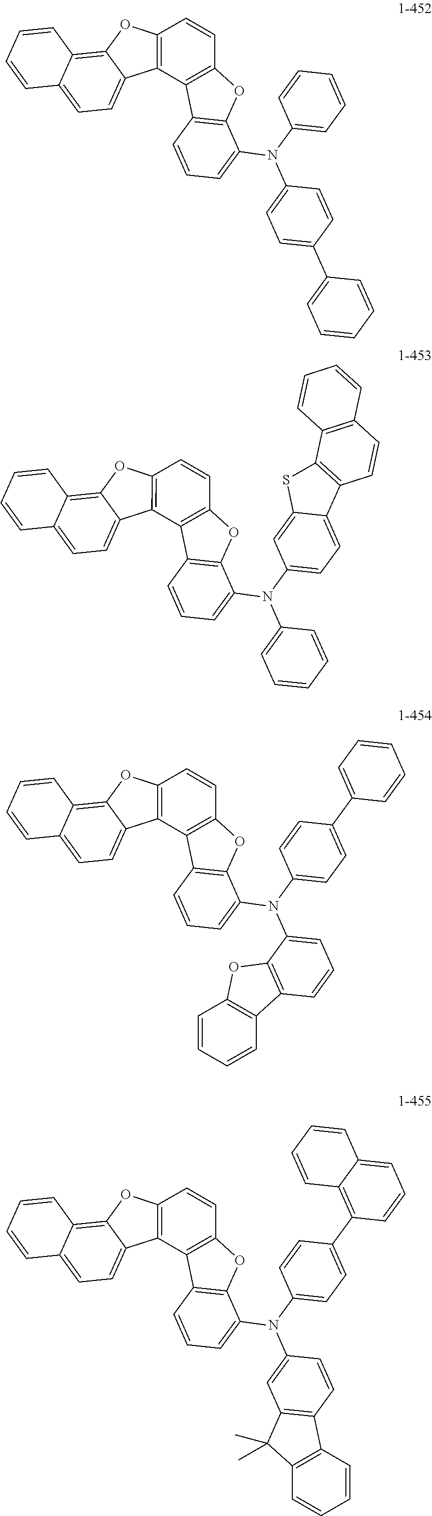

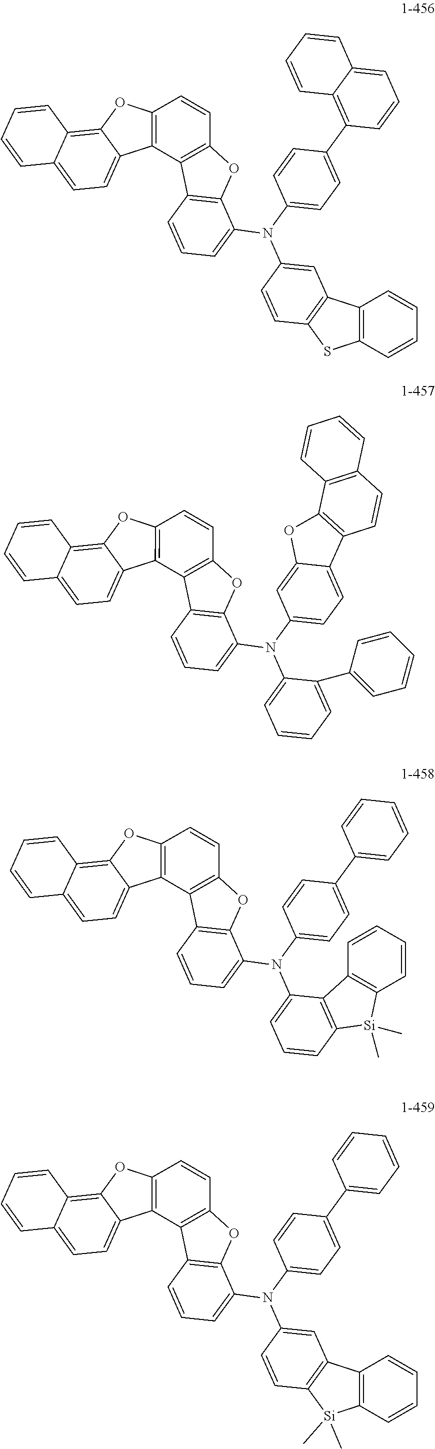 [SMILES: c1ccc2c(-c3ccc(N(c4ccc5sc6ccccc6c5c4)c4cccc5c4oc4ccc6oc7c8ccccc8ccc7c6c45)cc3)cccc2c1 ; c1ccc(-c2ccccc2N(c2ccc3c(c2)oc2c4ccccc4ccc32)c2cccc3c2oc2ccc4oc5c6ccccc6ccc5c4c23)cc1 ; C[Si]1(C)c2ccccc2-c2c(N(c3ccc(-c4ccccc4)cc3)c3cccc4c3oc3ccc5oc6c7ccccc7ccc6c5c34)cccc21 ; C[Si]1(C)c2ccccc2-c2cc(N(c3ccc(-c4ccccc4)cc3)c3cccc4c3oc3ccc5oc6c7ccccc7ccc6c5c34)ccc21]

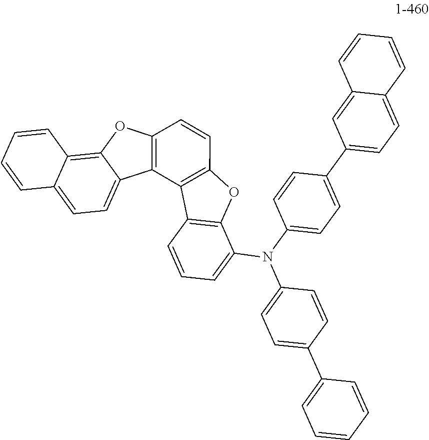 [SMILES: c1ccc(-c2ccc(N(c3ccc(-c4ccc5ccccc5c4)cc3)c3cccc4c3oc3ccc5oc6c7ccccc7ccc6c5c34)cc2)cc1]

8. A composition for an organic optoelectronic device, the composition comprising
a first compound; and
a second compound,
wherein;
the first compound is the compound for an organic optoelectronic device of claim 1, and
the second compound is represented by Chemical Formula 2: 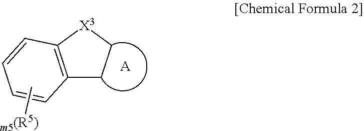
in Chemical Formula 2,
X³ is O, S, N—L^{a}-R^{a}, CR^{b}R^{c}, or SiR^{d}R^{e},
L^{a} is a single bond or a substituted or unsubstituted C6 to C12 arylene group,
R^{a}, R^{b}, R^{c}, R^{d}, R^{e}, and R⁵ are each independently hydrogen, deuterium, a substituted or unsubstituted C6 to C30 aryl group or a substituted or unsubstituted C2 to C30 heterocyclic group,
m5 is an integer of 1 to 4, and
A is a ring of Group II, 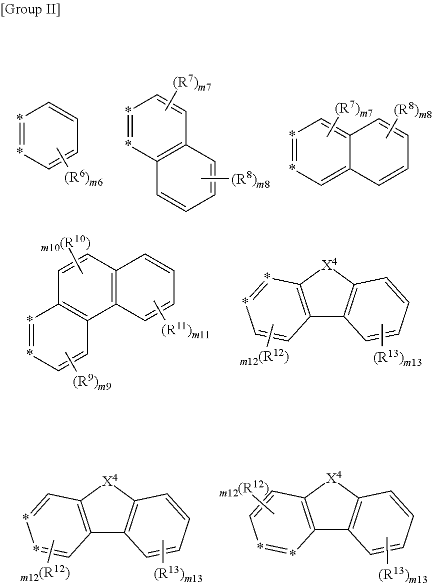
in Group II,
is a linking point,
X⁴ is O or S,
R⁶ to R¹³ are each independently hydrogen, deuterium, a substituted or unsubstituted C6 to C20 aryl group, or a substituted or unsubstituted C2 to C30 heterocyclic group,
m6, m8, m11, and m13 are each independently one of integers an integer of 1 to 4,
m7, m9, m10, and m12 are each independently an integer of 1 or 2, and at least one of R^{a} and R⁵ to R¹³ is a group represented by Chemical Formula a, 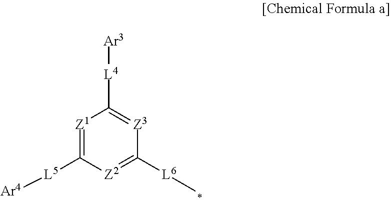
in Chemical Formula a,
Z¹ to Z³ are each independently N or CR^{f},
at least two of Z¹ to Z³ are N,
R^{f} is hydrogen, deuterium, a substituted or unsubstituted C1 to C30 alkyl group, or a substituted or unsubstituted C6 to C30 aryl group,
L⁴ to L⁶ are each independently a single bond or a substituted or unsubstituted C6 to C30 arylene group,
Ar³ and Ar⁴ are each independently a substituted or unsubstituted C6 to C30 aryl group or a substituted or unsubstituted C2 to C30 heteroaryl group, and
is a linking point.

9. The composition for an organic optoelectronic device of claim 8, wherein:
Chemical Formula 2 is represented by any one of Chemical Formula 2-I to Chemical Formula 2-X: 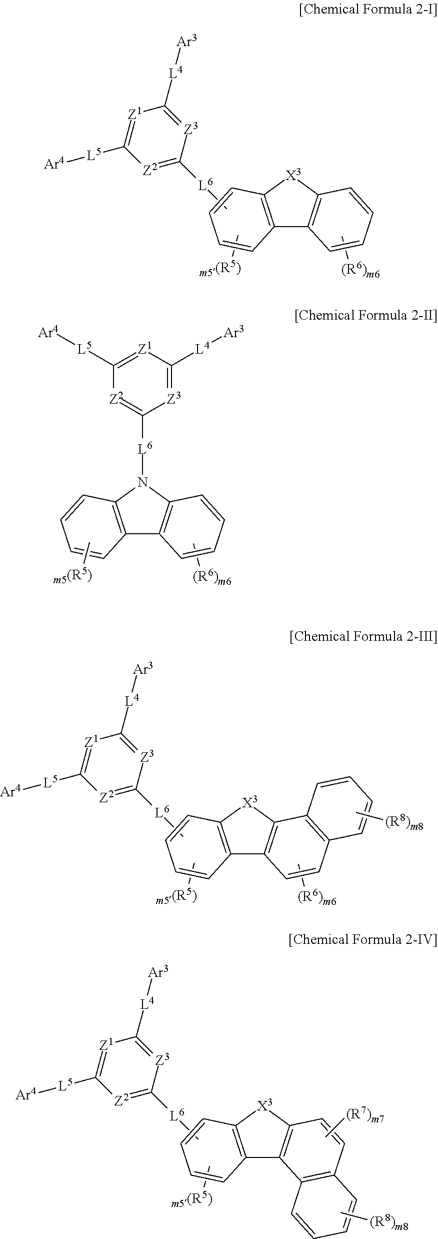 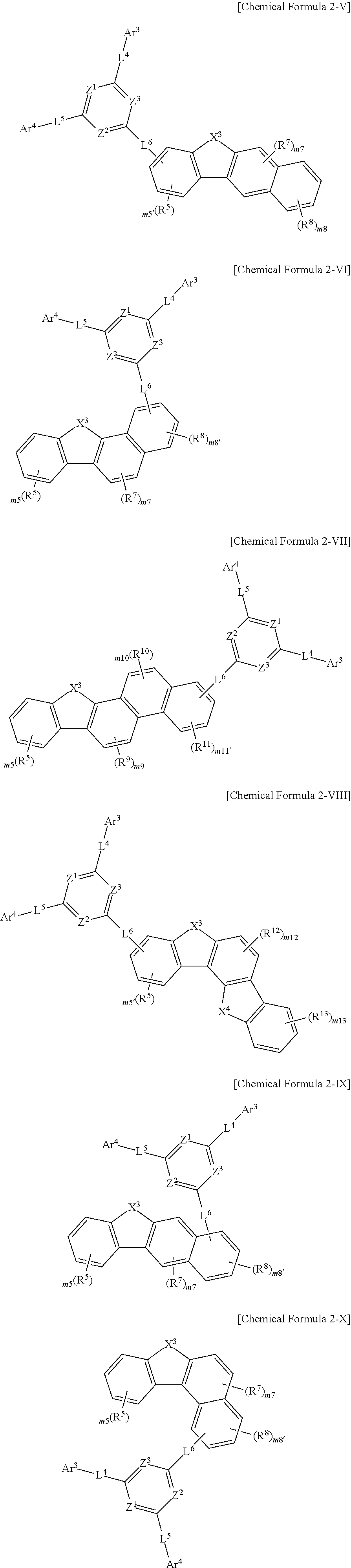
in Chemical Formula 2-I to Chemical Formula 2-X,
X³, X⁴, Z¹ to Z³, R⁵ to R¹³, m⁵ to m¹³, L⁴ to L⁶, Ar³, and Ar⁴ are defined the same as those of Chemical Formula 2 and Chemical Formula a, and
m5′, m8′, and m11′ are each independently an integer of 1 to 3.

10. The composition for an organic optoelectronic device of claim 9, wherein the second compound is represented by Chemical Formula 2-III or Chemical Formula 2-VI.

11. The composition for an organic optoelectronic device of claim 8, wherein:
the second compound is represented by Chemical Formula 2-III-1 or Chemical Formula 2-VI-1: 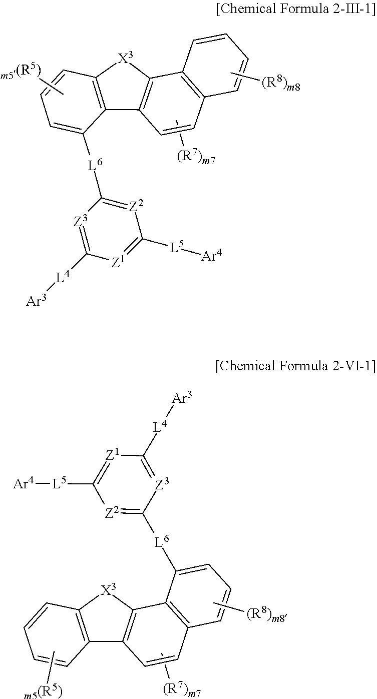
wherein, in Chemical Formula 2-III-1 and Chemical Formula 2-VI-1,
X³, Z¹ to Z³, R⁵, R⁷, R⁸, m5, m7, m8, L⁴ to L⁶, Ar³, and Ar⁴ are defined the same as those of Chemical Formula 2 and Chemical Formula a, and
m5′ and m8′ are each independently an integer of 1 to 3.

12. The composition for an organic optoelectronic device of claim 8, wherein Ar³ and Ar⁴ are each independently a substituted or unsubstituted phenyl group, a substituted or unsubstituted biphenyl group, a substituted or unsubstituted terphenyl group, a substituted or unsubstituted naphthyl group, a substituted or unsubstituted phenanthrenyl group, a substituted or unsubstituted triphenylene group, a substituted or unsubstituted carbazolyl group, a substituted or unsubstituted dibenzofuranyl group, a substituted or unsubstituted dibenzothiophenyl group, or a substituted or unsubstituted dibenzosilolyl group.

13. The composition for an organic optoelectronic device of claim 8, wherein the second compound is a compound of Group 2:

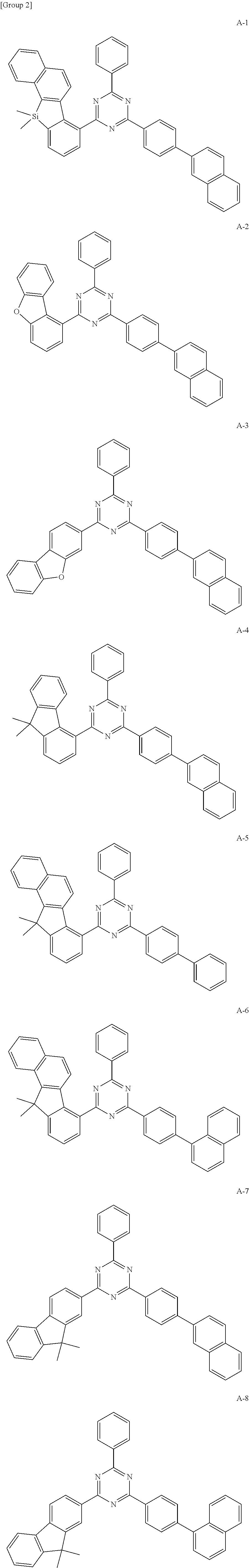

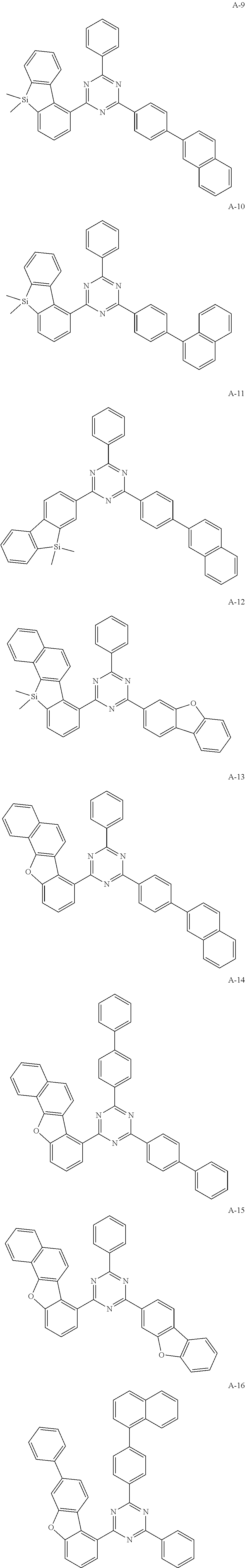

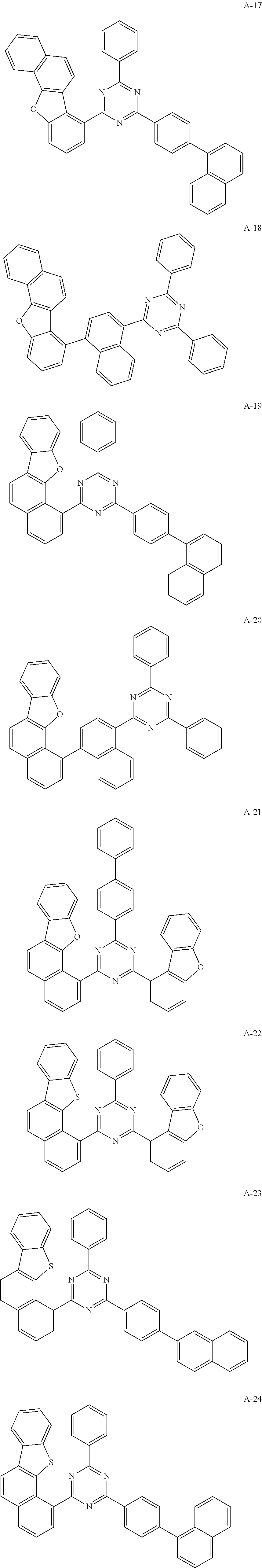

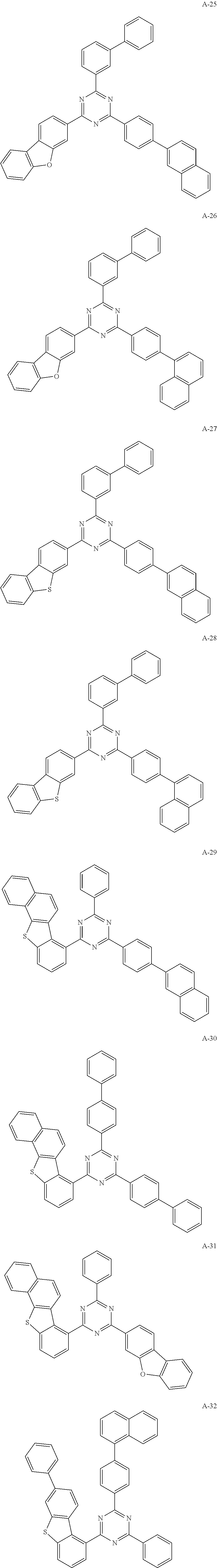

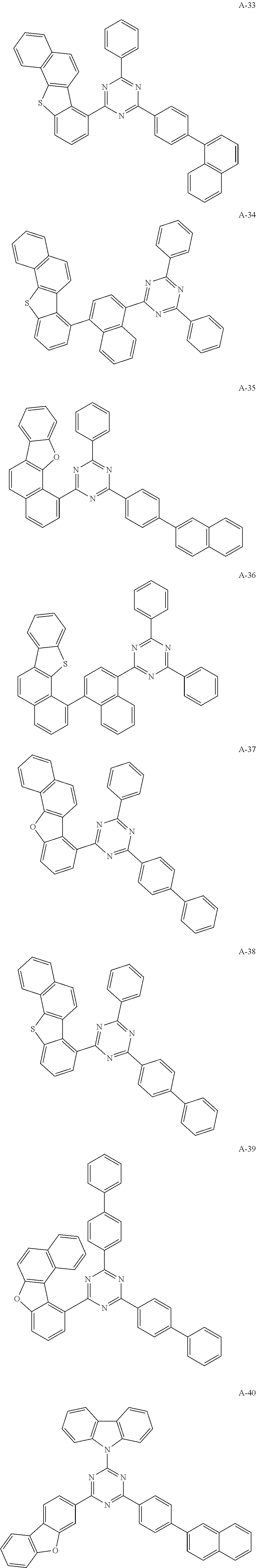

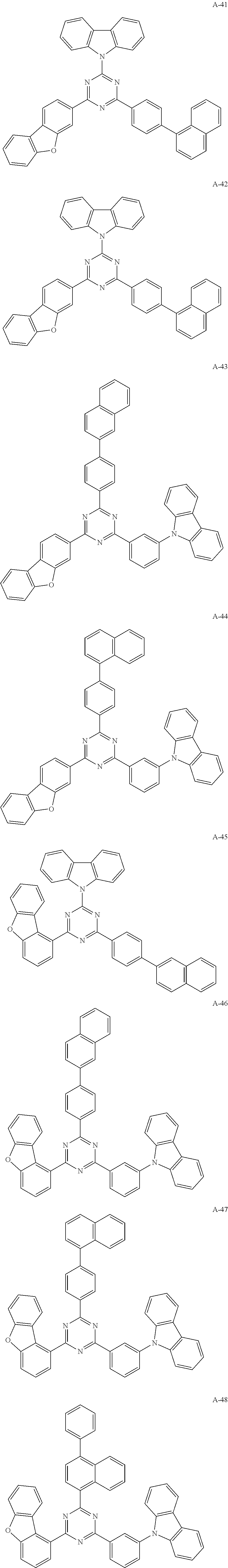

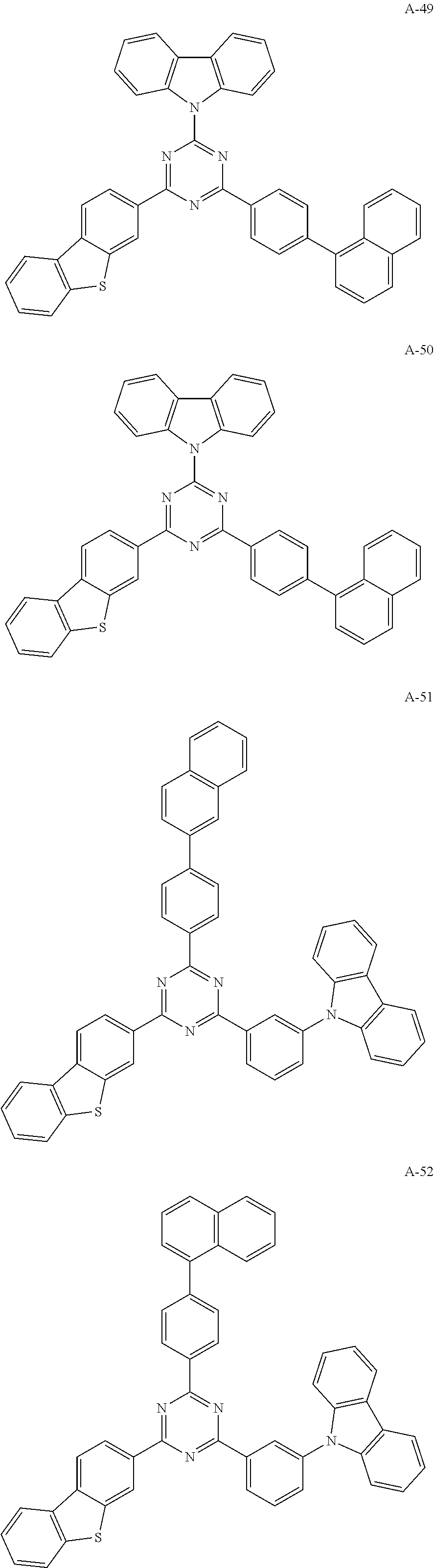 [SMILES: c1ccc2c(-c3ccc(-c4nc(-c5ccc6c(c5)sc5ccccc56)nc(-n5c6ccccc6c6ccccc65)n4)cc3)cccc2c1 ; c1ccc2c(-c3ccc(-c4nc(-c5ccc6c(c5)sc5ccccc56)nc(-n5c6ccccc6c6ccccc65)n4)cc3)cccc2c1 ; c1cc(-c2nc(-c3ccc(-c4ccc5ccccc5c4)cc3)nc(-c3ccc4c(c3)sc3ccccc34)n2)cc(-n2c3ccccc3c3ccccc32)c1 ; c1cc(-c2nc(-c3ccc(-c4cccc5ccccc45)cc3)nc(-c3ccc4c(c3)sc3ccccc34)n2)cc(-n2c3ccccc3c3ccccc32)c1]

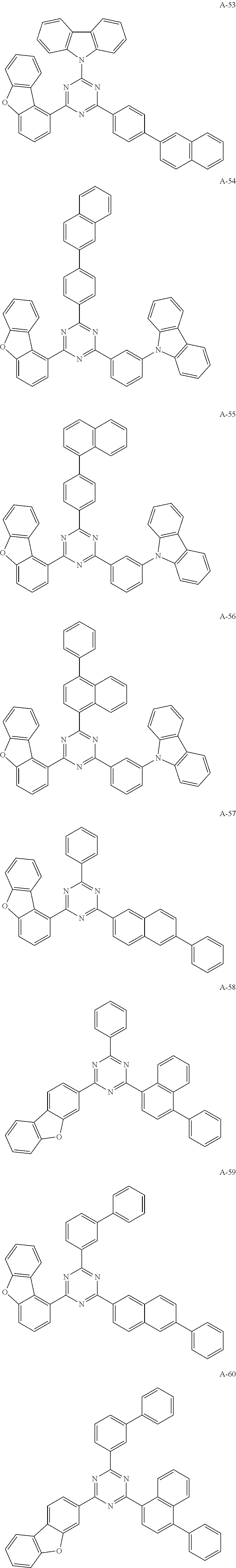

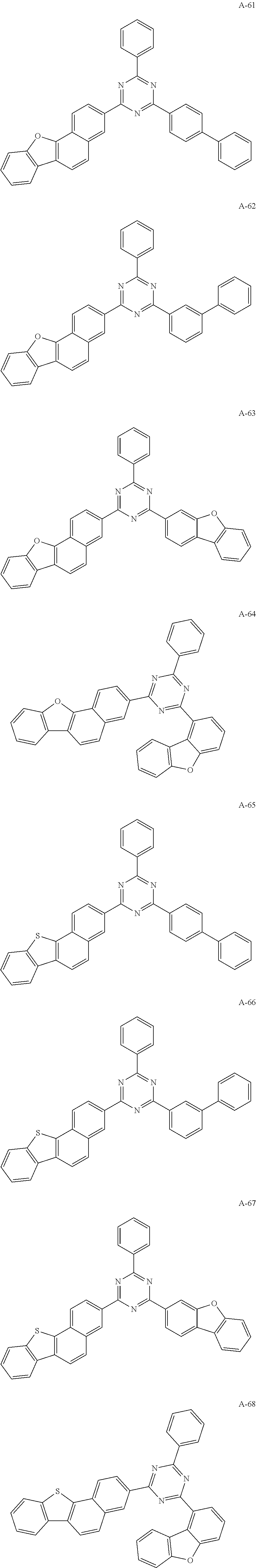

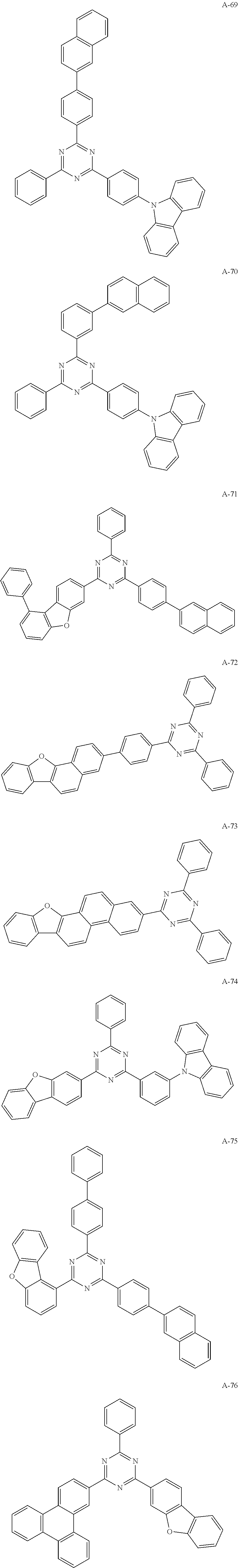

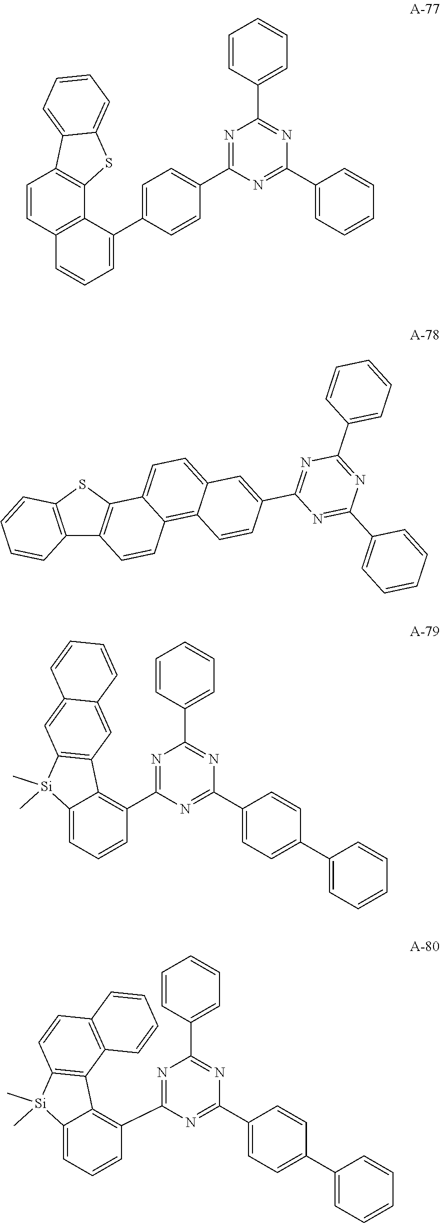 [SMILES: c1ccc(-c2nc(-c3ccccc3)nc(-c3ccc(-c4cccc5ccc6c7ccccc7sc6c45)cc3)n2)cc1 ; c1ccc(-c2nc(-c3ccccc3)nc(-c3ccc4c(ccc5c4ccc4c6ccccc6sc45)c3)n2)cc1 ; C[Si]1(C)c2cc3ccccc3cc2-c2c(-c3nc(-c4ccccc4)nc(-c4ccc(-c5ccccc5)cc4)n3)cccc21 ; C[Si]1(C)c2cccc(-c3nc(-c4ccccc4)nc(-c4ccc(-c5ccccc5)cc4)n3)c2-c2c1ccc1ccccc21]

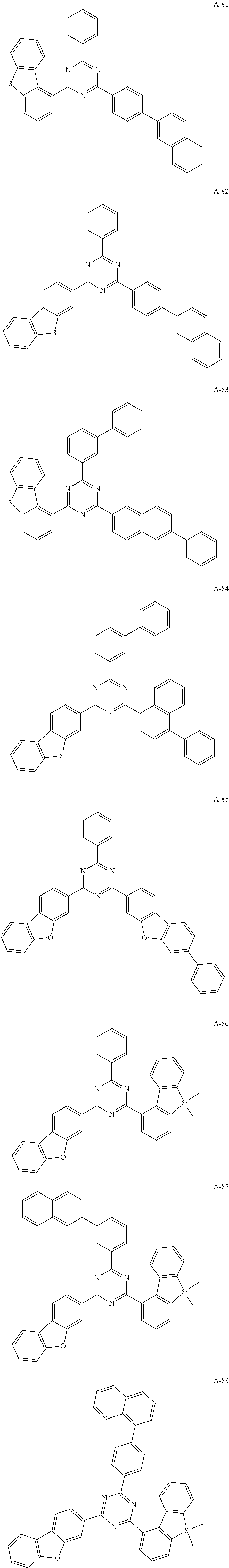

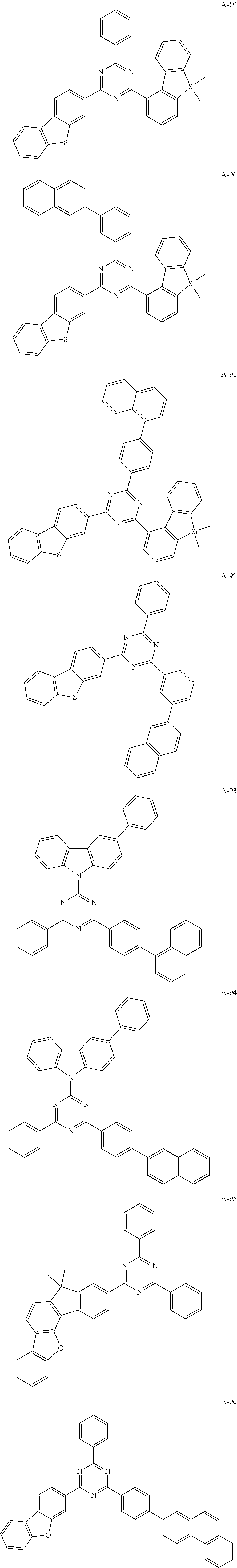

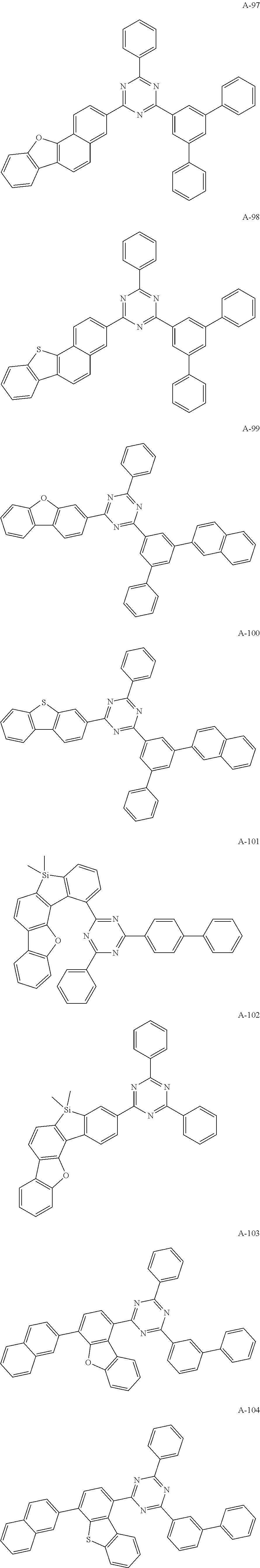

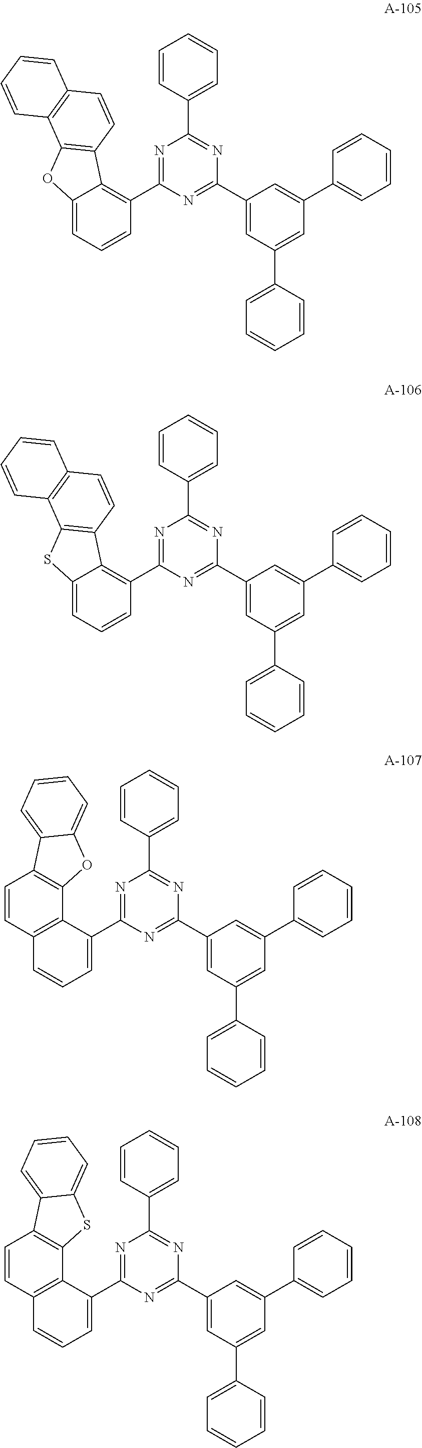 [SMILES: c1ccc(-c2cc(-c3ccccc3)cc(-c3nc(-c4ccccc4)nc(-c4cccc5oc6c7ccccc7ccc6c45)n3)c2)cc1 ; c1ccc(-c2cc(-c3ccccc3)cc(-c3nc(-c4ccccc4)nc(-c4cccc5sc6c7ccccc7ccc6c45)n3)c2)cc1 ; c1ccc(-c2cc(-c3ccccc3)cc(-c3nc(-c4ccccc4)nc(-c4cccc5ccc6c7ccccc7oc6c45)n3)c2)cc1 ; c1ccc(-c2cc(-c3ccccc3)cc(-c3nc(-c4ccccc4)nc(-c4cccc5ccc6c7ccccc7sc6c45)n3)c2)cc1]

14. An organic optoelectronic device, comprising:
an anode and a cathode facing each other, and
at least one organic layer between the anode and the cathode,
wherein the at least one organic layer includes the compound for the organic optoelectronic device of claim 1.

15. The organic photoelectronic device of claim 14, wherein
the at least one organic layer includes a light emitting layer, and
the light emitting layer includes the compound for an organic optoelectronic device.

16. A display device comprising the organic photoelectronic device of claim 14.

17. An organic optoelectronic device, comprising:
an anode and a cathode facing each other; and
at least one organic layer between the anode and the cathode,
wherein the at least one organic layer includes the composition for the organic optoelectronic device of claim 8.

18. The organic photoelectronic device of claim 17, wherein:
the at least one organic layer includes a light emitting layer, and
the light emitting layer includes the composition for an organic optoelectronic device.

19. A display device comprising the organic photoelectronic device of claim 17.
